(19) United States
(12) Patent Application Publication
(10) Pub. No.: US 2012/0172351 A1
(43) Pub. Date: Jul. 5, 2012

(54) NOVEL FUSED CYCLIC COMPOUND AND USE THEREOF

(76) Inventors: Nobuyuki Negoro, Kanagawa (JP); Yoshito Terao, Kanagawa (JP); Satoshi Mikami, Kanagawa (JP); Tomoya Yukawa, Kanagawa (JP)

(21) Appl. No.: 13/376,976

(22) PCT Filed: Jun. 8, 2010
(86) PCT No.: PCT/JP2010/059999

§ 371 Date: Mar. 23, 2012

Related U.S. Application Data
(60) Provisional application No. 61/213,448, filed on Jun. 9, 2009.
(60) Provisional application No. 61/272,980, filed on Nov. 30, 2009.

(51) Int. Cl. A61K 31/343; C07D 409/14; A61K 31/381; C07D 413/14; A61P 3/04; C07D 405/14; A61K 31/55; C07D 405/12; A61K 31/4184; A61P 3/10; C07D 407/12; A61K 31/5377

(52) U.S. Cl. 514/217.03; 549/467; 514/470; 549/60; 514/444; 544/153; 514/233.5; 540/596; 548/305.1; 514/394

(57) ABSTRACT

A compound represented by the formula (I): wherein each symbol is as defined in the specification, and a salt thereof have a GPR40 receptor activation action and is useful as an insulin secretagogue or a prophylactic or therapeutic drug for diabetes and the like.

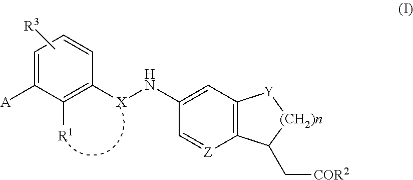

### TECHNICAL FIELD

The present invention relates to a novel fused ring compound having a GPR40 receptor activation action.

### BACKGROUND OF THE INVENTION

As a GPR40 receptor agonist useful a drug for the prophylaxis or treatment of diabetes and the like, the following compounds have been reported.

(1) WO2009/058237 describes the following compound as a GPR40 receptor agonist.

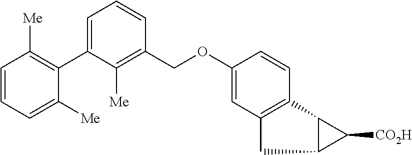

(2) WO2009/054423 describes the following compound as a GPR40 receptor agonist.

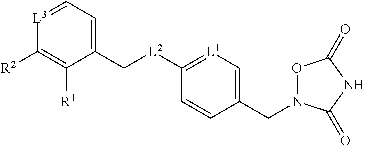

wherein L¹ and L³ are the same or different and each is CH or N, L² is O or NH, R¹ is —H or C₁₋₆ alkyl, R² is a group of the formula (II) or the formula (III),

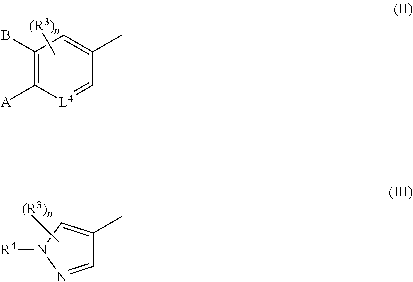

L⁴ is CH or N, A and B are the same or different and each is —O—(C₁₋₆ alkyl substituted by one or more groups selected from group G), amino optionally substituted by one or more groups selected from group G², —H or —R³ (wherein at least one of A and B is a group other than —H and —R³), R³ are the same or different and each is C₁₋₆ alkyl optionally substituted by one or more groups selected from the group consisting of —OH and halogen, halogen or —O—(C₁₋₆ alkyl), R⁴ is C₁₋₆ alkyl substituted by one or more groups selected from group G¹, n is 1 or 2, group G¹ is a group consisting of —NHCO₂R^{Z}, —NH₂, —NHCOR^{Z}, —NHCO-(cycloalkyl), —NHCO-(aryl), —NHSO₂R^{Z}, 1,3-dioxolan-4-yl optionally substituted by 1 to 5 C₁₋₆ alkyl, —OH, —OCOR^{Z}, —OR^{Z}, —CO₂R^{Z}, —CO₂H, —CONHR^{Z} and —CON(R^{Z})₂, group G² is a group consisting of —CO₂R^{Z} and —R^{Z}, R^{Z} are the same or different and each is C₁₋₆ alkyl optionally substituted by one or more groups selected from the group consisting of —OH and —OCO—(C₁₋₆ alkyl).
(3) WO2009/054390 describes the following compound as a GPR40 receptor agonist.

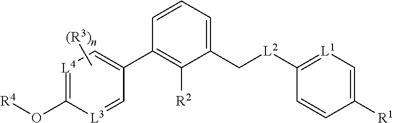

wherein R¹ is

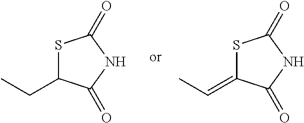

R² is —H or -lower alkyl, R³ is the same or different and each is lower alkyl optionally substituted by (optionally protected OH) or -halogen, n is 1 or 2, R⁴ is lower alkyl substituted by (optionally protected OH), L¹ is CH or N, L² is —O— or —NH—, L³ is CH or N, L⁴ is CH or N.
(4) WO2008/066097 describes the following compound as the GPR40 receptor agonist.

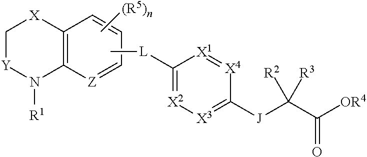

wherein R¹: —H, lower alkyl, halogeno lower alkyl, cycloalkyl, aryl, heterocyclic group, lower alkylene-R^{A}, —C(O)R^{B}, —CO₂R^{B} or —S(O)ₚR^{B}; lower alkylene, aryl or heterocyclic group for R¹ is optionally substituted; R^{A}: cycloalkyl, aryl, heterocyclic group, —S(O)ₚR^{O}, —S(O)ₚ-aryl, —S(O)ₚ-heterocyclic group, —C(O)R^{O}, —C(O)-aryl, —C(O)-heterocyclic group, —CO₂R^{O}, —OR^{O}, —O-aryl, —O-heterocyclic group, —N(R^{O})₂, —N(R^{O})-aryl, —N(R^{O})-heterocyclic group, —C(OR⁰) (aryl)₂, —C(O)N(R^{O})-cycloalkyl or —C(O)N(R^{O})-aryl; aryl or heterocyclic group for R^{A} is optionally substituted; R^{B}: lower alkyl, halogeno lower alkyl, cycloalkyl, aryl, heterocyclic group, lower alkylene-cycloalkyl, lower alkylene-aryl, lower alkylene-heterocyclic group, lower alkylene-OR^{O}, lower alkylene-O-aryl or lower alkylene-S(O)₂NH₂; aryl or heterocyclic group for R^{B} is optionally substituted; R⁰: —H or lower alkyl: n and p: the same or different and each is 0, 1 or 2; J: —C(R⁶)(R⁷)—, —O— or —S—; R², R³, R⁶ and R⁷: the same or different and each is —H, halogen, lower alkyl, —OR^{O}, or aryl; R² and R³, R³ and R⁶, and R⁶ and R⁷ each optionally form lower alkylene in combination; R⁴: —H or lower alkyl; X: single bond, —CH—, —(CH₂)₂—, —O—, —S—, —S(O)— or —S(O)₂—; Y: —CH— or —C(O)—; Z: C(—*), C(R⁸), N or N(O); * for Z means a bond to L; X¹ and X²: the same or different and each is C(R⁹), N or N(O); X³ and X⁴: the same or different and each is C(R¹⁰), N or N(O); R⁵: lower alkyl, halogen, halogeno lower alkyl, —OR^{O} or —O-halogeno lower alkyl; R⁸, R⁹ and R¹⁰: the same or different and each is —H, lower alkyl, halogen, halogeno lower alkyl, —OR^{O} or —O-halogeno lower alkyl; R⁶ and R¹⁰ optionally form lower alkylene, —O-lower alkylene or lower alkylene-O— in combination; L: —O-lower alkylene, lower alkylene-O—, —N(R¹¹)-lower alkylene, lower alkylene-N(R¹)—, —O-lower alkylene-O—, —N(R)-lower alkylene-N(R¹¹)—, —O-lower alkylene-N(R¹¹)— or —N(R¹¹)-lower alkylene-O—; R¹¹:—H, lower alkyl or —C(O)R^{O}.
(5) WO2007/123225 describes the following compound as a GPR40 receptor agonist.

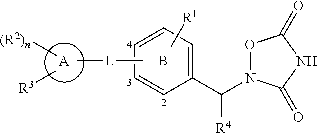

wherein R¹: —H, halogen, —R⁰, halogeno lower alkyl, —OR^{Z}, —S—R^{O} or —O-halogeno lower alkyl; R⁰: lower alkyl; R^{Z}: the same or different and each is —H or lower alkyl; L: *-lower alkylene-O—, *-lower alkylene-N(R^{Z})— or *—CON(R^{Z})—; * for L shows a bond to ring A; ring A: benzene, pyridine, thiophene, piperidine, dihydropyridine, pyrimidine or tetrahydroquinoline; ring B: benzene or pyridine; R²: the same or different and each is -halogen, —R⁰, halogeno lower alkyl, —OR^{Z}, —S—R⁰, —O-halogeno lower alkyl, —O-lower alkylene-aryl or oxo; n: 0, 1 or 2; R³: -halogen, —R⁰, -halogeno lower alkyl, —O—R⁰, —S—R^{O}, —O-halogeno lower alkyl, —X— (optionally substituted phenyl) or —X— (optionally substituted heteroaryl); X: single bond, 0, S or N(R^{Z}); R⁴: —H or lower alkyl; or R¹ and R⁴ optionally form lower alkylene in combination.
(6) WO2008/054675 describes the following compound as a GPR40 receptor agonist.

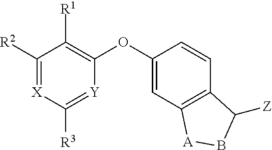

wherein A is selected from the group consisting of —CH₂—, —CF₂—, —O—, —N(R⁶)—, —S—, —S(O)—, —S(O)₂—, —C(═O)— and —CH(OH)—; B is selected from the group consisting of —CH₂—, —CH₂CH₂— and —CH(CH₃)—; or -A-B- is selected from the group consisting of —N(R⁶)C(═O)— and —C(═O)N(R⁶)—, or -A-B- shows two atoms that are bonded to form a 5-membered heteroaromatic ring having 1 to 3 hetero atoms independently selected from O, N and S, the 5-membered heteroaromatic ring may be substituted by 1 to 3 groups independently selected from halogen, CH₃, CF₃, —OCH₃ and —OCF₃; X is selected from ═C(R⁴)— and ═N—; Y is selected from ═C(R⁵)— and ═N—; X and Y are not simultaneously ═N—; a hetero ring is a 5- or 6-membered saturated or partially saturated monocyclic heterocycle having 1 to 3 hetero atoms independently selected from O, N and S; heteroaryl is a 5- or 6-membered monocyclic heteroaromatic ring having 1 to 3 hetero atoms independently selected from O, N and S; R¹, R², R³, R⁴ and R⁵ are independently selected from the group consisting of H, halogen, —CN, —NO₂, —C₁-C₆ alkyl, —OC₁-C₆ alkyl, —SC₁—C₆ alkyl, —S(O)₂C₁-C₆ alkyl, —N(R⁶)(R⁶), —N(R⁶)C(═O)C₁-C₆ alkyl, —N(R⁶)S(O)₂C₁-C₆ alkyl, —C(O)H, —C(═O)OH, —C(═O)OC₁-C₆ alkyl, —C(═O)C₁-C₆ alkyl, —C(═O)N(R⁶)(R⁶), phenylCH═CHC(═O)—, phenylC(═O)CH═CH—, —C(═O)phenyl, —C(═O)naphthyl, —C(═O)heterocycle, heterocycle, heteroaryl, C₃-C₇ cycloalkyl, phenyl and naphthyl; —C₁-C₆ alkyl, and alkyl group of —OC₁-C₆ alkyl, —SC₁-C₆ alkyl, —S(O)₂C₁-C₆ alkyl, —N(R⁶)C(═O)C₁-C₆ alkyl, —N(R⁶)S(O)₂C₁-C₆ alkyl, —C(═O)OC₁-C₆ alkyl and —C(═O)C₁-C₆ alkyl is optionally substituted by 1 to 5 halogens and optionally substituted by 1 or 2 groups independently selected from —OH, —OC₁-C₃ alkyl optionally substituted by 1 to 5 halogens, —S(O)₂C₁-C₃ alkyl, —C(═O)C₁-C₃ alkyl, —OC(O)C₁-C₃ alkyl, —NHC(═O)CH₃, —NHC(═O)OC₁-C₆ alkyl, —NHS(O)₂CH₃, —N(R⁶)(R⁶), heterocycle, heteroaryl, C₃-C₇ cycloalkyl, phenyl and naphthyl; phenyl, phenyl of phenylCHOHC(O)—, phenyl of phenylC(═O)CH═CH—, —C(═O)phenyl, —C(═O)naphthyl, —C(═O)heterocycle, heterocycle, heteroaryl, C₃-C₇ cycloalkyl, phenyl and naphthyl for any of R¹, R², R³, R⁴ and R⁵ or as a substituent for R¹, R², R³, R⁴ or R⁵ are optionally substituted by 1 to 4 substituents independently selected from halogen, —CN, —NO₂, —OH, —C₁-C₃ alkyl, —C(═O)C₁-C₃ alkyl, —S(O)₂C₁-C₃ alkyl and —OC₁-C₃ alkyl, and —C₁-C₃ alkyl, —OC₁-C₃ alkyl, —S(O)₂C₁-C₃ alkyl and —C(O)C₁-C₃ alkyl substituent is optionally substituted by 1 to 5 halogens; where necessary, the pair of substituents R¹ and R² in combination show a 3- or 4-carbon crosslinked group selected from —CH₂CH₂CH₂—, —CH₂CH₂CH₂CH₂— and —CH═CH—CHOH—, form condensed cyclopentyl, cyclohexyl or phenyl ring at the positions of R¹ and R², and the crosslinking group is optionally substituted by 1 to 3 groups independently selected from halogen, —OH, —CN, —NO₂, —C₁-C₃ alkyl, —OC₁-C₃ alkyl, —SC₁-C₃ alkyl, —S(O)₂C₁-C₃ alkyl, —CF₃ and —OCF₃; each R⁶ is independently selected from the group consisting of H and —C₁-C₆ alkyl; R⁷ is selected from the group consisting of H and —C₁-C₃ alkyl; R⁸ is selected from the group consisting of H, —OH, —C₁-C₃ alkyl optionally substituted by 1 to 3 halogens and —OC₁-C₃ alkyl optionally substituted by 1 to 3 halogens and optionally substituted by one —C(O)OR¹⁰ group; and R⁹ and R¹⁰ are independently selected from the group consisting of H and —C₁-C₆ alkyl, and —C₁-C₆ alkyl is optionally substituted by 1 to 5 halogens.
(7) WO2007/136573 describes the following compound as a GPR40 receptor agonist.

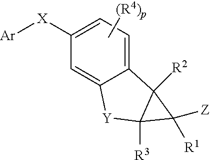

wherein Ar is selected from the group consisting of phenyl, naphthyl, a 5- or 6-membered monocyclic heteroaromatic group having 1 to 3 hetero atoms independently selected from O, N and S, and a benzoheteroaromatic group containing a phenyl group condensed with a 5- or 6-membered heteroaromatic ring having 1 to 3 hetero atoms independently selected from O, N and S; Ar is optionally substituted by 1 or 2 aromatic groups independently selected from phenyl, phenoxy, benzyl and a 5- or 6-membered heteroaromatic ring having 1 to 3 hetero atoms independently selected from O, N and S, and is optionally substituted by 1 to 5 substituents independently selected from halogen, —CN, —NO₂, —OH, —C(═O)H, —C(═O)OH, C₁₋₆ alkyl, —C₃₋₆ cycloalkyl, —OC₁₋₆ alkyl, —SC₁₋₆ alkyl, —C(═O)C₁₋₆ alkyl, —OC(═O)C₁₋₆ alkyl, —C(═O)OC₁₋₆ alkyl, —S(O)₂C₁₋₆ alkyl, —NR¹³R¹⁴, —C(═O)N(R¹³)(R¹⁴), —S(O)₂NR¹³R¹⁴ and —OC₃₋₆ cycloalkyl, (a) in all cases, C₁₋₆ alkyl is optionally substituted by 1 to 5 halogens, and optionally substituted by one group selected from —OC₁₋₄ alkyl optionally substituted by —OH and 1 to 5 halogens, (b) in all cases, C₃₋₆ cycloalkyl is optionally substituted by 1 or 2 substituents independently selected from halogen and CH₃, (c) aromatic substituents phenyl, phenoxy, benzyl and a 5- or 6-membered heteroaromatic ring having 1 to 3 hetero atoms independently selected from O, N and S are optionally substituted by 1 to 5 groups independently selected from halogen, —CN, —NO₂, —OH, —C(═O)H, —C(═O)OH, C₁₋₆ alkyl, —OC₁₋₆ alkyl, —SC₁₋₆ alkyl, —C(O)C₁₋₆ alkyl, —OC(O)C₁₋₆ alkyl, —C(O)OC₁₋₆ alkyl, —S(O)₂C₁₋₆ alkyl, NR¹³R¹⁴, —C(O)N(R¹³)(R¹⁴), —S(O)₂NR¹³R¹⁴ and —O(CH₂)_{q} (4- to 6-membered heterocycle having 1 or 2 hetero atoms independently selected from O, N and S), in all cases, C₁₋₆ alkyl is optionally substituted by 1 to 5 halogens, and optionally substituted by one group selected from —OC₁₋₄ alkyl optionally substituted by —OH and 1 to 5 halogens, and 4- to 6-membered heterocycle having 1 or 2 hetero atoms independently selected from O, N and S is optionally substituted by 1 or 2 groups independently selected from halogen, CH₃ and CF₃; X is selected from the group consisting of —O—, —S—, —S(O)—, —S(O)₂—, —NR⁵—, —OCR¹⁰R¹¹—, —SCR¹⁰R¹¹—, —NR⁵CR¹⁰R¹¹—, —CR¹⁰R¹¹O—, —CR¹⁰R¹¹S—, —CR¹⁰R¹¹NR⁵— and —CR⁶R⁷CR⁸R⁹O—; Y is selected from the group consisting of —O—, —S—, —S(O)—, —S(O)₂—, —NR⁵—, —C(═O)—, —CR⁶R⁷—, —OCR⁶R⁷—, —SCR⁶R⁷— and —CR⁶R⁷CR⁸R⁹—; Z is selected from the group consisting of —C(═O)OR¹², —C(═O)NR¹³R¹⁴ and 5-tetrazolyl; R¹, R² and R³ are each independently selected from the group consisting of H, halogen, C₁₋₃ alkyl and —OC₁₋₃ alkyl, C₁₋₃ alkyl and —OC₁₋₃ alkyl are each optionally substituted by 1 to 3 halogens; R⁴ is selected from the group consisting of halogen, —CN, —NO₂, —OH, —C(═O)H, —C(═O)OH, C₁₋₆ alkyl, —OC₁₋₆ alkyl, —SC₁₋₆ alkyl, —C(═O)C₁₋₆ alkyl, —OC(═O)C₁₋₆ alkyl, —C(O)OC₁₋₆ alkyl, —S(O)₂C₁₋₆ alkyl, —NR¹³R¹⁴, —C(═O)N(R¹³)(R¹⁴) and —S(O)₂NR¹³R¹⁴, in all cases, C₁₋₆ alkyl is optionally substituted by 1 to 5 halogens; R⁵, R¹³ and R¹⁴ are each independently selected from the group consisting of H, C₁₋₅ alkyl, —C(═O)C₁₋₅ alkyl and —S(O)₂C₁₋₅ alkyl, in all cases, C₁₋₅ alkyl is optionally substituted by 1 to 5 halogens; R⁶, R⁷, R⁸, R⁹, R¹⁰ and R¹¹ are each independently selected from the group consisting of H, halogen, —OH and C₁₋₃ alkyl optionally substituted by 1 to 5 halogens; R¹² is selected from the group consisting of H and C₁₋₇ alkyl optionally substituted by 1 to 5 halogens; p is an integer of 0 to 3; and q is 0 or 1.
(8) WO2006/083781 describes the following compound as a GPR40 receptor agonist.

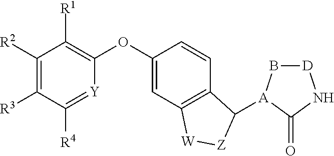

wherein A is selected from the group consisting of —CH— and —N—; B is selected from the group consisting of —S—, —O—, —NH—, —C(═O)— and —CH₂—; D is selected from the group consisting of —C(═O), —C(═S)—, —C(═NH)—, —O— and —NH—; W and Z are independently selected from —CH₂—, —CF₂—, —CH₂CH₂— and —CH₂CH₂CH₂—, one of W and Z is optionally selected from —O—, —C(═O), —NR⁶—, —S—, —S(O)— and —S(O)₂S; Y is selected from ═CH— and ═N—; a hetero ring is a 5- or 6-membered saturated or partially saturated monocyclic heterocycle having 1 to 3 hetero atoms independently selected from O, N and S; heteroaryl is a 5- or 6-membered monocyclic heteroaromatic ring having 1 to 3 hetero atoms independently selected from O, N and S; R¹, R², R³ and R⁴ are each independently selected from the group consisting of H, halogen, —CN, —NO₂, —C₁-C₆ alkyl, —OC₁-C₆ alkyl, —SC₁-C₆ alkyl, —S(O)₂C₁-C₆ alkyl, —N(R⁶)(R⁶), —N(R⁶)C(═O)C₁-C₆ alkyl, —N(R⁶)S(O)₂C₁-C₆ alkyl, —C(O)H, —C(═O)OH, —C(═O)OC₁-C₆ alkyl, —C(═O)C₁-C₆ alkyl, —C(═O)N(R⁶)(R⁶), —C(═O)phenyl, —C(═O)naphthyl, —C(═O)heterocycle, heterocycle, heteroaryl, C₃-C₇ cycloalkyl, phenyl and naphthyl; —C₁-C₆ alkyl, and alkyl group of —OC₁-C₆ alkyl, —SC₁-C₆ alkyl, —S(O)₂C₁-C₆ alkyl, —N(R⁶)C(═O)C₁-C₆ alkyl, —N(R⁶)S(O)₂C₁-C₆ alkyl, —C(═O)OC₁-C₆ alkyl and —C(═O)C₁-C₆ alkyl is optionally substituted by 1 to halogens, and optionally substituted by 1 or 2 groups independently selected from —OH, —OC₁-C₃ alkyl optionally substituted by 1 to 5 halogens, —CF₃, —S(O)₂C₁-C₃ alkyl, —C(═O)C₁-C₃ alkyl, —OC(O)C₁-C₃ alkyl, —NHC(═O)CH₃, —NHC(═O)OC₁-C₆ alkyl, —NHS(O)₂CH₃, —N(R⁶)(R⁶), heterocycle, heteroaryl, C₃-C₇ cycloalkyl, phenyl and naphthyl; —C(═O)phenyl, —C(═O)naphthyl, —C(═O)heterocycle, heterocycle, heteroaryl, C₃-C₇ cycloalkyl, phenyl or naphthyl as any of R¹, R², R³ and R⁴, or substituent of R¹, R², R³ or R⁴ is optionally substituted by 1 to 4 substituents independently selected from halogen, —CF₃, —OCF₃, —CN, —NO₂, —OH, —C₁-C₃ alkyl, —C(═O)C₁-C₃ alkyl, —S(O)₂C₁-C₃ alkyl and —OC₁-C₃ alkyl, —C₁-C₃ alkyl, —OC₁-C₃ alkyl, —S(O)₂C₁-C₃ alkyl and —C(O)C₁-C₃ alkyl substituent is optionally substituted by 1 to 3 halogens; where necessary, one pair of ortho substituents selected from (R¹-R²), (R²-R¹), (R²-R³), (R³-R²), (R³-R⁴) and (R⁴-R³) are bonded to form a divalent crosslinking group having a length of 3-5 atoms, the divalent crosslinking group is selected from —CH₂CH₂CH₂—, —CH₂CH₂CH₂CH₂—, —CH₂CH₂CH₂CH₂CH₂—, —OCH₂CH₂—, —OCH₂CH₂CH₂—, —OCH₂CH₂CH₂CH₂—, —CH₂OCH₂—, —CH₂OCH₂CH₂—, —CH₂OCH₂CH₂CH₂—, —CH₂CH₂OCH₂CH₂— and —SCH₂CH₂—, and the divalent crosslinking group is optionally substituted by 1 to 3 substituents independently selected from halogen, —OH, —CN, —NO₂, —C₁-C₃ alkyl, —OC₁-C₃ alkyl, —SC₁-C₃ alkyl, —S(O)₂C₁-C₃ alkyl, —CF₃ and —OCF₃; or the pair of ortho substituents R¹-R² are bonded by 4-carbon chain —CH═CH—CH═CH— to form condensed phenyl ring at the positions of R¹ and R², or bonded by 4-atom chain selected from —CH═CH—CH═N—, —N═CH—CH═CH—, —CH═N—CH═CH—, —CH═CH—N═CH—, —CH₂CH₂CH₂C(═O)— and —C(═O)CH₂CH₂CH₂— to optionally form condensed pyridinyl ring or condensed cyclohexanone ring at the positions of R¹ and R², the condensed phenyl ring, condensed pyridinyl ring and condensed cyclohexanone ring are optionally substituted by 1 to 3 substituents independently selected from halogen, —OH, —CN, —NO₂, —C₁-C₃ alkyl, —OC₁-C₃ alkyl, —SC₁-C₃ alkyl, —S(O)₂C₁-C₃ alkyl, —CF₃ and —OCF₃; or the pair of ortho substituents R¹-R² are optionally bonded by 3-atom chain selected from —CH═CHO—, —OCH═CH—, —CH═CH—S—, —SCH═CH—, —CH═CHN(R⁶)—, —N(R⁶)CH═CH—, —CH₂CH₂C(═O)— and —C(═O)CH₂CH₂— to form, at the positions of R¹ and R², 5-membered ring condensed with phenyl ring, the condensed 5-membered ring is optionally substituted by 1 to 3 substituents independently selected from halogen, —OH, —CN, —NO₂, —C₁-C₃ alkyl, —OC₁-C₃ alkyl, —SC₁-C₃ alkyl, —S(O)₂C₁-C₃ alkyl, —CF₃ and —OCF₃; and R⁶ is selected from the group consisting of H and —C₁-C₆ alkyl.
(9) WO2008/001931 describes the following compound as a GPR40 receptor agonist.

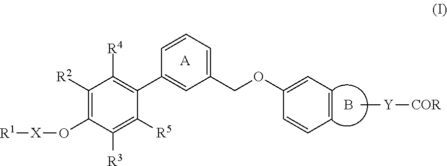

wherein R¹ is R⁶—SO₂— (R⁶ is a substituent) or an optionally substituted 1,1-dioxidotetrahydrothiopyranyl group; X is a bond or a divalent hydrocarbon group; R² and R³ are the same or different and each is a hydrogen atom, a halogen atom, an optionally substituted hydrocarbon group or an optionally substituted hydroxy group; R⁴ and R⁵ are the same or different and each is a C₁₋₆ alkyl group optionally substituted by a hydroxy group; ring A is a benzene ring further optionally having substituent(s) selected from a halogen atom, an optionally substituted hydrocarbon group, an optionally substituted hydroxy group and an optionally substituted amino group; ring B is a 5- to 7-membered ring; Y is a bond or CH₂; and R is an optionally substituted hydroxy group.
(10) WO2005/087710 describes the following compound as a GPR40 receptor agonist.

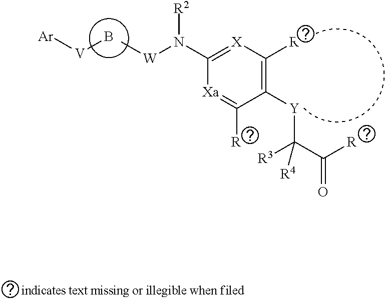

wherein Ar is an optionally substituted cyclic group other than a 4-piperidinyl group, ring B is an optionally substituted ring other than a thiazole ring and an oxanole ring), V is a bond or a spacer (excluding —N═N—) having an atom number of the main chain of 1 to 3, W is a bond or an C₁₋₆ alkylene group optionally substituted by a C₁₋₆ alkoxy group, X and Xa are the same or different and each is CH or N, Y is O or CR⁶R⁷ (R⁶ and R⁷ are the same or different and each is a hydrogen atom, a halogen atom, a C₁₋₆ alkyl group or an optionally substituted hydroxy group, and R⁷ is bonded to R^{1a} to form a 4- to 8-membered ring), R¹ and R^{1a} are the same or different and each is a hydrogen atom, a halogen atom, a C₁₋₆ alkyl group or a C₁₋₆ alkoxy group, R² is a hydrogen atom, a C₁₋₆ alkyl group or an optionally substituted acyl group, R³ and R⁴ are the same or different and each is a hydrogen atom or a halogen atom, R⁵ is an optionally substituted hydroxy group or an optionally substituted amino group, provided when W is a bond, then ring B is an optionally substituted benzene ring condensed nonaromatic ring other than a tetrahydroquinoline ring.
(11) WO2005/063725 describes the following compound as a GPR40 receptor agonist.

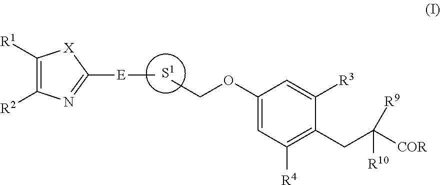

wherein X is S or O, R¹ and R² are the same or different and each is a hydrogen atom, an optionally substituted C₆₋₁₄ aryl group, an optionally substituted heterocyclic group or an optionally substituted C₁₋₆ alkyl group, R¹ and R² are bonded to form a ring together with the carbon atom bonded thereto, E is —W¹—N(R⁵)—W²—, —W¹—CH(R⁶)—O—W²—, —W¹—O—CH(R⁶)—W²—, —W—S(O)ₙ—W²— or —W¹—CH(R⁶)—W²— (W¹ and W² are the same or different and each is a bond or an optionally substituted C₁₋₃ alkylene group, R⁵ and R⁶ are each an optionally substituted heterocyclic group or an optionally substituted hydrocarbon group, n is 1 or 2, provided that when X is S, then R⁵ and R⁶ are not C₁₋₆ alkyl groups, ring S¹ is a benzene ring or a pyridine ring optionally further having substituent(s) selected from an optionally substituted C₁₋₆ alkyl group, an optionally substituted C₁₋₆ alkoxy group and a halogen atom, R³ and R⁴ are the same or different and each is a hydrogen atom, a halogen atom, an optionally substituted C₁₋₆ alkyl group or an optionally substituted C₁₋₆ alkoxy group, R⁹ and R¹⁰ are the same or different and each is a hydrogen atom, a halogen atom or a C₁₋₆ alkoxy group, and R is an optionally substituted hydroxy group or optionally substituted amino group.
(12) WO2005/063729 describes the following compound as a GPR40 receptor agonist.

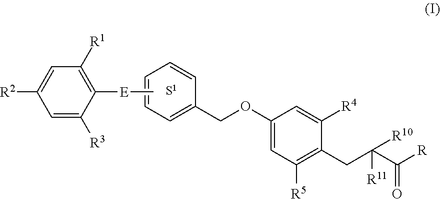

wherein R¹, R³, R⁴ and R⁵ are the same or different and each is a hydrogen atom, a halogen atom, an optionally substituted hydrocarbon group or an optionally substituted hydroxy group; R² is a halogen atom, a nitro group, an optionally substituted hydrocarbon group, an optionally substituted hydroxy group, an optionally substituted amino group, an optionally substituted mercapto group, an optionally substituted acyl group or an optionally substituted heterocyclic group; R¹⁰ and R¹¹ are the same or different and each is a hydrogen atom, a halogen atom or a C₁₋₆ alkoxy group; E is a bond, an optionally substituted C₁₋₄ alkylene group, —W¹—O—W²—, —W¹—S—W²— or —W¹—N(R⁶)—W²— (W¹ and W² are the same or different and each is a bond or an optionally substituted C₁₋₃ alkylene group, and R⁶ is a hydrogen atom, an optionally substituted acyl group or an optionally substituted hydrocarbon group); ring S¹ is a benzene ring further optionally having substituent(s) selected from a halogen atom, an optionally substituted hydrocarbon group, an optionally substituted hydroxy group and an optionally substituted amino group; and R is an optionally substituted hydroxy group or an optionally substituted amino group; provided R¹ and R³ are not simultaneously hydrogen atoms.
(13) WO2004/106276 describes the following compound as a GPR40 receptor agonist.

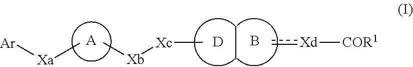

wherein Ar is an optionally substituted cyclic group, ring A is an optionally further substituted ring other than thiazole, oxazole, imidazole and pyrazole, Xa and Xb are independently a bond or a spacer having an atom number of the main chain of 1 to 5, Xc is O, S, SO or SO₂,

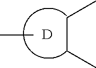

is

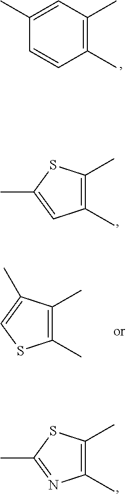

ring B is a 5- to 7-membered ring, Xd is a bond, CH or CH₂, . . . is a single bond when Xd is a bond or CH₂, a double bond when Xd is CH, and R¹ is an optionally substituted hydroxy group, provided that (i) when ring A is benzene, a cyclic group for Ar is not a quinolinyl group, (ii) when ring B is a 5- to 7-membered aromatic ring, a ring for ring A is not thiophene or furan, (iii) when ring B is benzene, a ring for ring A is not 5-membered aromatic heterocycle, (iv) when ring B is cyclohexane, Xd is not a bond.
(14) WO2004/041266 describes the following compound as a GPR40 receptor agonist.

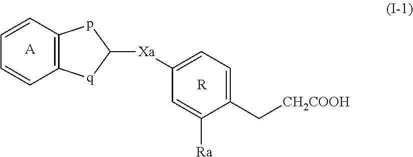

wherein ring A is an optionally substituted benzene ring, ring R is an optionally substituted phenylene group, Xa is a spacer other than an alkylene group, p and q are each an optionally substituted carbon chain having a carbon number of 0 to 4, and Ra is a hydrogen atom or a substituent.

As other compounds which are not GPR40 receptor agonists, the following compounds have been reported.

(15) WO2005/009975 describes the following compound as an MEK inhibitor.

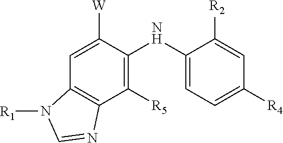

wherein W is

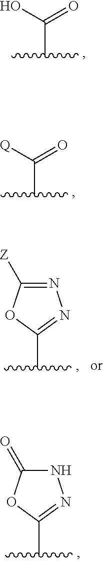

Q is —O—R₃, —NH₂, —NH[(CH₂)ₖCH₃] or —NH[O(CH₂)ₖCH₃](—NH₂ is optionally substituted by 1 or 2 substituents independently selected from methyl and —NR₉R₉ₐ, and —(CH₂)ₖCH₃ moiety of —NH[(CH₂)ₖCH₃] and —NH[O(CH₂)ₖCH₃] group is optionally substituted by 1 to 3 substituents independently selected from —OH, —NR₉R₉ₐ, C₁₋₆ alkyl and C₃-C₁₂ cycloalkyl); Z is —NH₂, —NH[(CH₂)ₖCH₃] or —NH[O(CH₂)ₖCH₃](—NH₂ is optionally substituted by 1 or 2 substituents independently selected from methyl and —NR₉R₉ₐ, and —(CH₂)ₖCH₃ moiety of —NH[(CH₂)ₖCH₃] and —NH[O(CH₂)ₖCH₃] group is optionally substituted by 1 to 3 substituents independently selected from —OH and —NR₉R₉ₐ); R₁ is hydrogen, C₁₋₆ alkyl, C₂₋₆ alkenyl, C₂₋₆alkynyl, C₃-C₁₂ cycloalkyl, —(CR₁₀R₁₁)_{q}(C₆-C₁₀ aryl) or —(CR₁₀R₁₁)_{q} (4- to 10-membered heterocycle); R₁ is optionally substituted by 1 to 3 substituents selected from the group consisting of —COOH, —COOR₁₄, —COR₉, —(CR₁₀R₁₁)_{q}(C₆-C₁₀ aryl), —(CR₁₀R₁₁)_{q}(4-10-membered heterocycle), —SO₂R₁₁, —SO₂NR₁₂R₁₃, —OH, —OR₁₄, cyano, halo, —NR₉R₉ₐ and —NR₉CO(R₁₁); R₂ is hydrogen, chlorine, fluorine or methyl; R₃ is C₁₋₆ alkyl; R₄ is bromine, chlorine, fluorine, iodine, C₁₋₆ alkyl, C₂₋₄alkenyl, C₂₋₆alkynyl, C₃₋₆ cycloalkyl, —(CH₂)—C₃₋₆cycloalkyl, cyano, —O—(C₁₋₄ alkyl), —S—(C₁₋₂ alkyl), —SOCH₃, —SO₂CH₃, —SO₂NR₆R₇, —C≡C—(CH₂)ₙNH₂, —C≡C—(CH₂)ₙNHCH₃, —C≡C—(CH₂)ₙN(CH₃)₂, —C≡C—CH₂OCH₃, —C═C(CH₂)ₙOH, —C═C—(CH₂)ₙNH₂, —CHCHCH₂OCH₃, —CHCH—(CH₂)ₙNHCH₃, —CHCH—(CH₂)ₙN(CH₃)₂, —(CH₂)ₚCO₂R₆, —C(O)C₁₋₃ alkyl, —C(O)NHCH₃, —(CH₂)ₘNH₂, —(CH₂)ₘNHCH₃, —(CH₂)ₘN(CH₃)₂, —(CH₂)ₘOR₆, —CH₂S(CH₂)ₜ (CH₃), —(CH₂)ₚCF₃, —C≡CCF₃, —CH═CHCF₃, —CH₂CHCF₂, —CH═CF₂, —(CF₂)ᵥCF₃, —CH₂(CF₂)ₙCF₃, —(CH₂)ₜCF(CF₃)₂, —CH(CF₃)₂, —CF₂CF(CF₃)₂ or —C(CF₃)₃ (C₁₋₆ alkyl and C₂₋₆alkynyl are optionally substituted by 1 to 3 substituents independently selected from —OH and C₁₋₆ alkyl); R₅ is hydrogen or fluorine; R₆ and R₇ are each independently hydrogen, methyl or ethyl; R₉ₐ and R₉ are each independently hydrogen or C₁₋₆ alkyl; k is 0 to 3; m is 1 to 4; n is 1 to 2; p is 0 to 2; t is 0 or 1; and v is 1 to 5.
(16) WO2004/014382 and (17) WO2003/063794 describe the following compound as IgE/IgG receptor modulator.

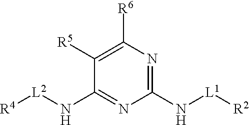

wherein L¹ and L² are each independently selected from the group consisting of a direct bond and a linker; R² is selected from the group consisting of (C₁-C₆)alkyl optionally substituted by one or more, the same or different R⁸ groups, (C3-C8)cycloalkyl optionally substituted by one or more, the same or different R⁸ groups, cyclohexyl optionally substituted by one or more, the same or different R⁸ groups, 3- to 8-membered cycloheteroalkyl optionally substituted by one or more, the same or different R⁸ groups, (C5-C15)aryl optionally substituted by one or more, the same or different R⁸ groups, phenyl optionally substituted by one or more, the same or different R⁸ groups and 5- to 15-membered heteroaryl optionally substituted by one or more, the same or different R⁸ groups; R⁴ is selected from the group consisting of hydrogen, (C1-C6)alkyl optionally substituted by one or more, the same or different R⁸ groups, (C3-C8)cycloalkyl optionally substituted by one or more, the same or different R⁸ groups, cyclohexyl optionally substituted by one or more, the same or different R⁸ groups, 3- to 8-membered cycloheteroalkyl optionally substituted by one or more, the same or different R⁸ groups, (C5-C15)aryl optionally substituted by one or more, the same or different R⁸ groups, phenyl optionally substituted by one or more, the same or different R⁸ groups and 5- to 15-membered heteroaryl optionally substituted by one or more, the same or different R⁸ groups; R⁵ is selected from the group consisting of R⁶, (C1-C6)alkyl optionally substituted by one or more, the same or different R⁸ groups, (C1-C4)alkanyl optionally substituted by one or more, the same or different W groups, (C2-C4)alkenyl optionally substituted by one or more, the same or different R⁸ groups and (C2-C4)alkynyl optionally substituted by one or more, the same or different R⁸ groups; each R⁶ is independently selected from the group consisting of hydrogen, electric negative group, —OR^{d}, —SR^{d}, (C1-C3)haloalkyloxy, (C1-C3)perhaloalkyloxy, —NR^{c}R^{c}, halogen, (C1-C3)haloalkyl, (C1-C3)perhaloalkyl, —CF₃, —CH₂CF₃, —CF₂CF₃, —CN, —NC, —OCN, —SCN, —NO, —NO₂, —N₃, —S(O)R^{d}, —S(0)₂R^{d}, —S(0)₂0R^{d}, —S(O)NR^{c}R^{c}; —S(0)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NRcRc, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙNR^{c}R^{c} and —[NHC(NH)]ₙNR^{c}R^{c}, (C5-C10)aryl optionally substituted by one or more, the same or different R⁸ groups, phenyl optionally substituted by one or more, the same or different R⁸ groups, (C6-C16)arylalkyl optionally substituted by one or more, the same or different R⁸ groups, 5- to 10-membered heteroaryl optionally substituted by one or more, the same or different R⁸ groups and 6- to 16-membered heteroarylalkyl optionally substituted by one or more, the same or different R⁸ groups; R⁸ is selected from the group consisting of R^{a}, R^{b}, R^{a} substituted by one or more, the same or different R^{a} or R^{b}, —OR^{a} substituted by one or more, the same or different R^{a} or R^{b}, —B(OR^{a})₂, —B(NR^{c}R^{c})₂, —(CH₂)ₘ—Rb, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b) 2}, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b}; each R^{a} is independently selected from the group consisting of hydrogen, (C1-C6)alkyl, (C3-C8)cycloalkyl, cyclohexyl, (C4-C11)cycloalkylalkyl, (C5-C10)aryl, phenyl, (C6-C16)arylalkyl, benzyl, 2- to 6-membered heteroalkyl, 3- to 8-membered cycloheteroalkyl, morpholinyl, piperazinyl, homopiperazinyl, piperidinyl, 4- to 11-membered cycloheteroalkylalkyl, 5- to 10-membered heteroaryl and 6- to 16-membered heteroarylalkyl; each R^{b} is an appropriate group independently selected from the group consisting of ═O, —OR^{d}, (C1-C3)heteroalkyloxy, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}—[NR^{a}C(O)]ₙOR^{d}, —[NHC(O)]ₙNR^{c}R^{c}, —[NR^{a}C(O)]ₙNR^{c}R^{c}, —[NHC(NH)ₙNR^{c}R^{c} and —[NR^{a}C(NR^{a})]ₙNR^{c}R^{c}; each R^{c} is independently a protecting group or R^{a}, or each R^{C} form, together with the nitrogen atom bonded thereto, 5- to 8-membered cycloheteroalkyl or heteroaryl, optionally further contains one or more, the same or different hetero atoms, or optionally substituted by one or more, the same or different R^{a} or appropriate R^{h} group; each R^{d} is independently R^{a}; each m is independently an integer of 1 to 3; each n is independently an integer of 0 to 3; provided (1) when L¹ is a direct bond and R⁶ is hydrogen, R² is not 3,4,5-tri(C1-C6)alkoxyphenyl; (2) when L¹ and L are each a direct bond and R² is substituted phenyl, and R⁶ is hydrogen, R⁵ is other than cyano or —C(O)NHR (R is hydrogen or (C1-C6)alkyl); (3) when L¹ and L² are each a direct bond, and R² and R⁴ are each independently substituted or unsubstituted pyrrole or indole, then R and R⁴ are bonded to molecule residue via a ring carbon atom; (4) the compound is not a compound of the following formula

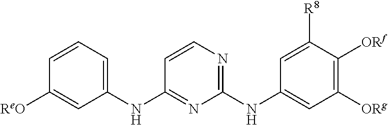

wherein R^{e} is (C1-C6)alkyl; R^{f} and R^{g} are each independently straight chain or branched chain (C1-C6)alkyl optionally substituted by one or more, the same or different R⁸ groups; and R⁸ is as defined above.

However, none of these documents specifically disclose the compound of the present invention.

### SUMMARY OF THE INVENTION

### Problems to be Solved by the Invention

The present invention aims to provide a novel fused ring compound having a GPR40 receptor activation action and useful as an insulin secretagogue or a drug for the prophylaxis or treatment of diabetes and the like.

### Means of Solving the Problems

The present inventors have intensively conducted various studies and found that the compound represented by the following formula (I) unexpectedly has a superior GPR40 receptor agonist activity, shows superior properties as pharmaceutical products such as stability, particularly, high dissolution property, low toxicity, fine pharmacokinetics such as sustainability in blood and the like, and therefore, it can be a safe and useful medicament for the prophylaxis or treatment of GPR40 receptor-related pathology or diseases in mammals, based on which the present inventors have completed the present invention.

Accordingly, the present invention relates to [1] a compound represented by the formula (I):

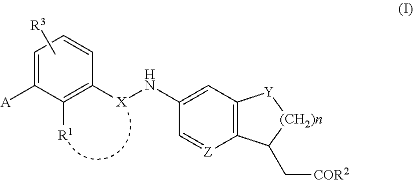

wherein R¹ is a halogen atom, hydroxy, optionally substituted C₁₋₆ alkyl or optionally substituted C₁₋₆ alkoxy,
R² is optionally substituted hydroxy,
R³ is a hydrogen atom, a halogen atom or optionally substituted C₁₋₆ alkyl,
X is CH₂ (wherein R¹ and X optionally form an optionally substituted ring),

### Y is CH₂, NH or O,

### Z is CH or N,

n is an integer selected from 1 to 3,
A is a halogen atom, optionally substituted amino, or a 4- to 13-membered cyclic group optionally substituted by 1 to 5 substituents selected from
(1) a halogen atom,
(2) optionally substituted amino,
(3) optionally substituted C₁₋₆ alkylthio,
(4) optionally substituted C₁₋₆ alkyl,
(5) optionally substituted C₃₋₁₀ cycloalkyl,
(6) optionally substituted C₁₋₆ alkoxy,
(7) optionally substituted C₆₋₁₄ aryl,
(8) an optionally substituted 4- to 7-membered heterocyclic group, and
(9) optionally substituted 4- to 7-membered heterocyclyl-oxy or a salt thereof (hereinafter to be sometimes abbreviated as compound (I));
[1A] a compound represented by the formula (I):

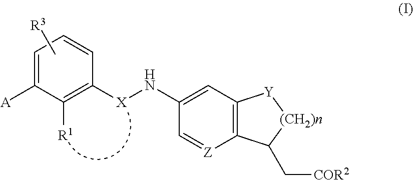

wherein R¹ is a halogen atom, hydroxy, optionally substituted C₁₋₆ alkyl or optionally substituted C₁₋₆ alkoxy,
R² is optionally substituted hydroxy,
R³ is a hydrogen atom, a halogen atom or optionally substituted C₁₋₆ alkyl,
X is CH₂ (wherein R¹ and X optionally form an optionally substituted ring),

### Y is CH₂, NH or O,

### Z is CH or N,

n is an integer selected from 1 to 3,
A is a halogen atom, optionally substituted amino, or a 4- to 13-membered cyclic group optionally substituted by 1 to 3 substituents selected from
(1) a halogen atom,
(2) optionally substituted amino,
(3) optionally substituted C₁₋₆ alkylthio,
(4) optionally substituted C₁₋₆ alkyl,
(5) optionally substituted C₃₋₁₀ cycloalkyl,
(6) optionally substituted C₁₋₆ alkoxy,
(7) optionally substituted C₆₋₁₄ aryl,
(8) an optionally substituted 4- to 7-membered heterocyclic group, and
(9) optionally substituted 4- to 7-membered heterocyclyl-oxy, or a salt thereof;
[2] the compound or salt of the above-mentioned [1] or [1A] wherein R¹ is C₁₋₆ alkyl (wherein R¹ and X optionally form an optionally substituted ring);
[3] the compound or salt of the above-mentioned [1] represented by the formula (II):

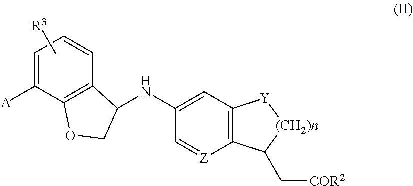

wherein R² is optionally substituted hydroxy,
R³ is a hydrogen atom, a halogen atom or optionally substituted C₁₋₆ alkyl,

### Y is CH₂, NH or O,

### Z is CH or N,

n is an integer selected from 1 to 3,
A is a halogen atom, optionally substituted amino, or a 4- to 13-membered cyclic group optionally substituted by 1 to 5 substituents selected from
(1) a halogen atom,
(2) optionally substituted amino,
(3) optionally substituted C₁₋₆ alkylthio,
(4) optionally substituted C₁₋₆ alkyl,
(5) optionally substituted C₃₋₁₀ cycloalkyl,
(6) optionally substituted C₁₋₆ alkoxy,
(7) optionally substituted C₆₋₁₄ aryl,
(8) an optionally substituted 4- to 7-membered heterocyclic group, and
(9) optionally substituted 4- to 7-membered heterocyclyl-oxy (hereinafter to be sometimes abbreviated as compound (II));
[3A] the compound or salt of the above-mentioned [1], [2], [3] or [1A], which is represented by the formula (II):

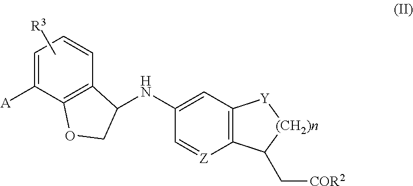

wherein R² is optionally substituted hydroxy,
R³ is a hydrogen atom, a halogen atom or optionally substituted C₁₋₆ alkyl,

### Y is CH₂, NH or O,

### Z is CH or N,

n is an integer selected from 1 to 3,
A is a halogen atom, optionally substituted amino, or a 4- to 13-membered cyclic group optionally substituted by 1 to 3 substituents selected from
(1) a halogen atom,
(2) optionally substituted amino,
(3) optionally substituted C₁₋₆ alkylthio,
(4) optionally substituted C₁₋₆ alkyl,
(5) optionally substituted C₃₋₁₀ cycloalkyl,
(6) optionally substituted C₁₋₆ alkoxy,
(7) optionally substituted C₆₋₁₄ aryl,
(8) an optionally substituted 4- to 7-membered heterocyclic group, and
(9) optionally substituted 4- to 7-membered heterocyclyl-oxy;
[4] the compound or salt of the above-mentioned [1], [2], [3], [1A] or [3A], wherein R² is hydroxy;
[5] the compound or salt of the above-mentioned [1], [2], [3], [4], [1A] or [3A], wherein R³ is a hydrogen atom or C₁₋₆ alkyl;
[6] the compound or salt of the above-mentioned [1], [2], [3], [4], [5], [1A] or [3A], wherein Y is O;
[7] the compound or salt of the above-mentioned [1], [2], [3], [4], [5], [6], [1A] or [3A], wherein Z is CH;
[8] the compound or salt of the above-mentioned [1], [2], [3], [4], [5], [6], [7], [1A] or [3A], wherein n is 1;
[9] the compound or salt of the above-mentioned [1], [2], [3], [4], [5], [6], [7] or [8], wherein A is phenyl, benzimidazolyl or a 5- or 6-membered aromatic heterocyclic group (preferably, thienyl, pyrimidinyl) each optionally substituted by 1 to 5 substituents selected from
(a) a halogen atom,
(b) C₁₋₆ alkyl optionally substituted by 1 to 3 halogen atoms,
(c) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio,
   (d) a 4- to 7-membered heterocyclic group, and
   (e) 4- to 7-membered heterocyclyl-oxy optionally substituted by 1 to 3 substituents selected from C₁₋₆ alkyl and oxo;
   [10] the compound or salt of the above-mentioned [1], [2], [3], [4], [5], [6], [7], [8] or [9], wherein R² is hydroxy,
   R³ is a hydrogen atom or C₁₋₆ alkyl,

### Y is O,

### Z is CH,

n is 1,
A is phenyl, benzimidazolyl or a 5- or 6-membered aromatic heterocyclic group (preferably, thienyl, pyrimidinyl) each optionally substituted by 1 to 5 substituents selected from
(a) a halogen atom,
(b) C₁₋₆ alkyl optionally substituted by 1 to 3 halogen atoms,
(c) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio,
   (d) a 4- to 7-membered heterocyclic group, and
   (e) 4- to 7-membered heterocyclyl-oxy optionally substituted by 1 to 3 substituents selected from C₁₋₆ alkyl and oxo;
   [11] the compound or salt of the above-mentioned [1], [2], [3], [4], [5], [6], [7], [8], [9], [10], [1A] or [3A], wherein A is phenyl optionally substituted by 1 to 3 substituents selected from
   (a) C₁₋₆ alkyl optionally substituted by 1 to 3 halogen atoms,
   (b) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio, and
   (c) 4- to 7-membered heterocyclyl-oxy optionally substituted by 1 to 3 substituents selected from C₁₋₆ alkyl and oxo;
   [12] the compound or salt of the above-mentioned [1], [2], [3], [4], [5], [6], [7], [8], [9], [10], [1A] or [3A], wherein A is benzimidazolyl or a 5- or 6-membered aromatic heterocyclic group (preferably, thienyl, pyrimidinyl) each optionally substituted by 1 to 3 substituents selected from
   (a) a halogen atom,
   (b) C₁₋₆ alkyl optionally substituted by 1 to 3 halogen atoms, and
   (c) a 5- or 6-membered heterocyclic group;
   [13] [(3S)-6-{[(3S)-7-{2,6-dimethyl-4-[3-(methylsulfonyl)propoxy]phenyl}-2,3-dihydro-1-benzofuran-3-yl]amino}-2,3-dihydro-1-benzofuran-3-yl]acetic acid or a salt thereof;
   [14] [(3S)-6-{[(3S)-7-{4-[(1,1-dioxidotetrahydro-2H-thiopyran-4-yl)oxy]-2,6-dimethylphenyl}-2,3-dihydro-1-benzofuran-3-yl]amino}-2,3-dihydro-1-benzofuran-3-yl]acetic acid or a salt thereof;
   [15] [(3S)-6-{[(3S)-7-(2-ethyl-6,7-difluoro-1H-benzimidazol-1-yl)-2,3-dihydro-1-benzofuran-3-yl]amino}-2,3-dihydro-1-benzofuran-3-yl]acetic acid or a salt thereof;
   [16] [(3S)-6-{[(3S)-7-(2-ethoxy-6,7-difluoro-1H-benzimidazol-1-yl)-2,3-dihydro-1-benzofuran-3-yl]amino}-2,3-dihydro-1-benzofuran-3-yl]acetic acid or a salt thereof;
   [17] [(3S)-6-({(3S)-7-[4,6-dimethyl-2-(morpholin-4-yl)pyrimidin-5-yl]-2,3-dihydro-1-benzofuran-3-yl}amino)-2,3-dihydro-1-benzofuran-3-yl]acetic acid or a salt thereof;
   [18] a prodrug of the compound or salt of the above-mentioned [1], [2], [3], [4], [5], [6], [7], [8], [9], [10], [11], [12], [13], [14], [15], [16], [17], [1A] or [3A];
   [19] a medicament comprising the compound or salt of the above-mentioned [1], [2], [3], [4], [5], [6], [7], [8], [9], [10], [11], [12], [13], [14], [15], [16], [17], [1A] or [3A], or a prodrug thereof;
   [20] the medicament of the above-mentioned [19] for activating a GPR40-mediated signal;
   [21] the medicament of the above-mentioned [19], which is an agent for the prophylaxis or treatment of diabetes or obesity;
   [22] a method of activating a GPR40-mediated signal, comprising administering the compound or salt of the above-mentioned [1], [2], [3], [4], [5], [6], [7], [8], [9], [10], [11], [12], [13], [14], [15.], [16], [17], [1A] or [3A] or a prodrug thereof to a mammal;
   [23] a method for the prophylaxis or treatment of diabetes or obesity, comprising administering the compound or salt of the above-mentioned [1], [2], [3], [4], [5], [6], [7], [8], [9], [10], [11], [12], [13], [14], [15], [16], [17], [1A] or [3A] or a prodrug thereof to a mammal;
   [24] use of the compound or salt of the above-mentioned [1], [2], [3], [4], [5], [6], [7], [8], [9], [10], [11], [12], [13], [14], [15], [16], [17], [1A] or [3A] or a prodrug thereof for the production of a medicament that activates a GPR40-mediated signal;
   [25] use of the compound or salt of the above-mentioned [1], [2], [3], [4], [5], [6], [7], [8], [9], [10], [11], [12], [13], [14], [15], [16], [17], [1A] or [3A] or a prodrug thereof for the production of an agent for the prophylaxis or treatment of diabetes or obesity;
   and the like.

### Effect of the Invention

The present compound (I) has a superior GPR40 receptor agonist activity, and further has superior properties as a pharmaceutical product such as stability and the like. Particularly, since the compound shows high dissolution property, low toxicity, good kinetics such as sustainability in blood and the like, it can be a safe and useful drug for the prophylaxis or treatment of GPR40 receptor-related pathology or diseases in mammals.

### DETAILED DESCRIPTION OF THE INVENTION

The present invention is explained in detail in the following.

Unless otherwise specified, as the “halogen atom” in the present specification, a fluorine atom, a chlorine atom, a bromine atom and an iodine atom can be mentioned.

Unless otherwise specified, as the “optionally substituted hydrocarbon” in the present specification, for example, “optionally substituted C₁₋₆ alkyl”, “optionally substituted C₂₋₆alkenyl”, “optionally substituted C₂₋₆alkynyl”, “optionally substituted C₃₋₈cycloalkyl”, “optionally substituted C₆₋₁₄ aryl”, “optionally substituted C₇₋₁₆ aralkyl” and the like can be mentioned.

Unless otherwise specified, as the “C₁₋₆ alkyl” in the present specification, for example, methyl, ethyl, propyl, isopropyl, butyl, isobutyl, sec-butyl, tert-butyl, pentyl, isopentyl, neopentyl, hexyl and the like can be mentioned.

Unless otherwise specified, as the “C₂₋₆ alkenyl” in the present specification, for example, vinyl, propenyl, isopropenyl, 2-buten-1-yl, 4-penten-1-yl, 5-hexen-1-yl and the like can be mentioned.

Unless otherwise specified, as the “C₂₋₆alkynyl” in the present specification, for example, 2-butyn-1-yl, 4-pentyn-1-yl, 5-hexyn-1-yl and the like can be mentioned.

Unless otherwise specified, as the “C₃₋₈cycloalkyl” in the present specification, for example, cyclopropyl, cyclobutyl, cyclopentyl, cyclohexyl and the like can be mentioned.

Unless otherwise specified, as the “C₆₋₁₄ aryl” in the present specification, for example, phenyl, 1-naphthyl, 2-naphthyl, 2-biphenylyl, 3-biphenylyl, 4-biphenylyl, 2-anthryl and the like can be mentioned. The C₆₋₁₄ aryl may be saturated partially, and as the partially saturated C₆₋₁₄ aryl, for example, tetrahydronaphthyl and the like can be mentioned.

Unless otherwise specified, as the “C₇₋₁₆ aralkyl” in the present specification, for example, benzyl, phenethyl, diphenylmethyl, 1-naphthylmethyl, 2-naphthylmethyl, 2,2-diphenylethyl, 3-phenylpropyl, 4-phenylbutyl, 5-phenylpentyl, 2-biphenylylmethyl, 3-biphenylylmethyl, 4-biphenylylmethyl and the like can be mentioned.

Unless otherwise specified, as the “optionally substituted hydroxy” in the present specification, for example, “hydroxy”, “optionally substituted C₁₋₆ alkoxy”, “optionally substituted heterocyclyl-oxy”, “optionally substituted C₆₋₁₄ aryloxy”, “optionally substituted C₇₋₁₆ aralkyloxy” and the like can be mentioned.

Unless otherwise specified, as the “C₁₋₆ alkoxy” in the present specification, for example, methoxy, ethoxy, propoxy, isopropoxy, butoxy, isobutoxy, tert-butoxy, pentyloxy, hexyloxy and the like can be mentioned.

Unless otherwise specified, as the “C₁₋₆ alkoxy-C₁₋₆ alkoxy” in the present specification, for example, methoxymethoxy, methoxyethoxy, ethoxymethoxy, ethoxyethoxy and the like can be mentioned.

As the “heterocyclyl-oxy” in the present specification, a hydroxy substituted by a “heterocyclic group” below can be mentioned. As preferable examples of the heterocyclyl-oxy group, tetrahydropyranyloxy, thiazolyloxy, pyridyloxy, pyrazolyloxy, oxazolyloxy, thienyloxy, furyloxy and the like can be mentioned.

Unless otherwise specified, as the “C₆₋₁₄ aryloxy” in the present specification, for example, phenoxy, 1-naphthyloxy, 2-naphthyloxy and the like can be mentioned.

Unless otherwise specified, as the “C₇₋₁₆ aralkyloxy” in the present specification, for example, benzyloxy, phenethyloxy and the like can be mentioned.

Unless otherwise specified, as the “optionally substituted mercapto” in the present specification, for example, “mercapto”, “optionally substituted C₁₋₆ alkylthio”, “optionally substituted heterocyclyl-thio”, “optionally substituted C₆₋₁₄ arylthio”, “optionally substituted C₇₋₁₆ aralkylthio” and the like can be mentioned.

Unless otherwise specified, as the “C₁₋₆ alkylthio” in the present specification, for example, methylthio, ethylthio, propylthio, isopropylthio, butylthio, sec-butylthio, tert-butylthio and the like can be mentioned.

Unless otherwise specified, as the “heterocyclyl-thio” in the present specification, mercapto substituted by a “heterocyclic group” below can be mentioned. As preferable examples of the heterocyclyl-thio, tetrahydropyranylthio, thiazolylthio, pyridylthio, pyrazolylthio, oxazolylthio, thienylthio, furylthio and the like can be mentioned.

Unless otherwise specified, as the “C₆₋₁₄ arylthio” in the present specification, for example, phenylthio, 1-naphthylthio, 2-naphthylthio and the like can be mentioned.

Unless otherwise specified, as the “C₇₋₁₆ aralkylthio” in the present specification, for example, benzylthio, phenethylthio and the like can be mentioned.

Unless otherwise specified, as the “heterocyclic group” in the present specification, for example, a 5- to 14-membered (monocyclic, bicyclic or tricyclic) heterocyclic group containing, as a ring-constituting atom besides carbon atoms, one or two kinds of 1 to 4 hetero atoms selected from a nitrogen atom, a sulfur atom and an oxygen atom, preferably (i) a 5- to 14-membered (preferably 5- to 10-membered) aromatic heterocyclic group, (ii) a 5- to 10-membered non-aromatic heterocyclic group and the like can be mentioned. Of these, a 5- or 6-membered aromatic heterocyclic group is preferable. Specifically, aromatic heterocyclic groups such as thienyl (e.g., 2-thienyl, 3-thienyl), furyl (e.g., 2-furyl, 3-furyl), pyridyl (e.g., 2-pyridyl, 3-pyridyl, 4-pyridyl), thiazolyl (e.g., 2-thiazolyl, 4-thiazolyl, 5-thiazolyl), oxazolyl (e.g., 2-oxazolyl, 4-oxazolyl, 5-oxazolyl), pyrazinyl, pyrimidinyl (e.g., 2-pyrimidinyl, 4-pyrimidinyl), pyrrolyl (e.g., 1-pyrrolyl, 2-pyrrolyl, 3-pyrrolyl), imidazolyl (e.g., 1-imidazolyl, 2-imidazolyl, 4-imidazolyl), pyrazolyl (e.g., 1-pyrazolyl, 3-pyrazolyl, 4-pyrazolyl), triazolyl (e.g., 1-triazolyl, 2-triazolyl), tetrazolyl, pyridazinyl (e.g., 3-pyridazinyl, 4-pyridazinyl), isothiazolyl (e.g., 3-isothiazolyl, 4-isothiazolyl, 5-isothiazolyl), isoxazolyl (e.g., 3-isoxazolyl, 4-isoxazolyl, 5-isoxazolyl), indolyl (e.g., 1-indolyl, 2-indolyl, 3-indolyl), 2-benzothiazolyl, 2-benzoxazolyl, benzimidazolyl (e.g., 1-benzimidazolyl, 2-benzimidazolyl), benzo[b]thienyl (e.g., 2-benzo[b]thienyl, 3-benzo[b]thienyl), benzo[b]furyl (e.g., 2-benzo[b]furanyl, 3-benzo[b]furanyl), quinolyl (e.g., 2-quinolyl, 3-quinolyl, 4-quinolyl, 5-quinolyl, 8-quinolyl), isoquinolyl (e.g., 1-isoquinolyl, 3-isoquinolyl, 4-isoquinolyl, 5-isoquinolyl), and the like;

non-aromatic heterocyclic groups such as pyrrolidinyl (e.g., 1-pyrrolidinyl, 2-pyrrolidinyl, 3-pyrrolidinyl), oxazolidinyl (e.g., 2-oxazolidinyl), imidazolinyl (e.g., 1-imidazolinyl, 2-imidazolinyl, 4-imidazolinyl), piperidinyl (e.g., piperidino, 2-piperidinyl, 3-piperidinyl, 4-piperidinyl), piperazinyl (e.g., 1-piperazinyl, 2-piperazinyl), morpholinyl (e.g., 2-morpholinyl, 3-morpholinyl, morpholino), thiomorpholinyl (e.g., 2-thiomorpholinyl, 3-thiomorpholinyl, thiomorpholino), tetrahydropyranyl and the like,
and the like can be mentioned.

Unless otherwise specified, as the “C₁₋₆ alkyl-carbonyl” in the present specification, for example, acetyl, isobutanoyl, isopentanoyl and the like can be mentioned.

Unless otherwise specified, as the “C₁₋₆ alkoxy-carbonyl” in the present specification, for example, methoxycarbonyl, ethoxycarbonyl, propoxycarbonyl, tert-butoxycarbonyl and the like can be mentioned.

Unless otherwise specified, as the “C₃₋₈cycloalkyl-carbonyl” in the present specification, for example, cyclopentylcarbonyl, cyclohexylcarbonyl and the like can be mentioned.

Unless otherwise specified, as the “C₆₋₁₄ aryl-carbonyl” in the present specification, for example, benzoyl, 1-naphthoyl, 2-naphthoyl and the like can be mentioned.

Unless otherwise specified, as the “C₇₋₁₆ aralkyl-carbonyl” in the present specification, for example, phenylacetyl, 2-phenylpropanoyl and the like can be mentioned.

Unless otherwise specified, as the “C₆₋₁₄ aryloxy-carbonyl” in the present specification, for example, phenoxycarbonyl, naphthyloxycarbonyl and the like can be mentioned.

Unless otherwise specified, as the “C₇₋₁₆ aralkyloxy-carbonyl” in the present specification, for example, benzyloxycarbonyl, phenethyloxycarbonyl and the like can be mentioned.

Unless otherwise specified, as the “nitrogen-containing heterocyclyl-carbonyl” in the present specification, for example, pyrrolidinylcarbonyl, piperidinocarbonyl and the like can be mentioned.

Unless otherwise specified, as the “C₁₋₆ alkylsulfonyl” in the present specification, for example, methylsulfonyl, ethylsulfonyl and the like can be mentioned.

Unless otherwise specified, as the “C₆₋₁₄ arylsulfonyl” in the present specification, for example, phenylsulfonyl, 1-naphthylsulfonyl, 2-naphthylsulfonyl and the like can be mentioned.

Unless otherwise specified, as the “C₁₋₆ alkylsulfinyl” in the present specification, for example, methylsulfinyl, ethylsulfinyl and the like can be mentioned.

Unless otherwise specified, as the “C₆₋₁₄ arylsulfinyl” in the present specification, for example, phenylsulfinyl, 1-naphthylsulfinyl, 2-naphthylsulfinyl and the like can be mentioned.

Unless otherwise specified, as the “optionally esterified carboxyl” in the present specification, for example, carboxyl, C₁₋₆ alkoxy-carbonyl, C₆₋₁₄ aryloxy-carbonyl, C₇₋₁₆ aralkyloxy-carbonyl and the like can be mentioned.

Unless otherwise specified, as the “optionally halogenated C₁₋₆ alkyl” in the present specification, the above-mentioned “C₁₋₆ alkyl” optionally substituted by 1 to 5 above-mentioned “halogen atoms” can be mentioned. For example, methyl, ethyl, propyl, isopropyl, butyl, tert-butyl, isobutyl, trifluoromethyl and the like can be mentioned.

Unless otherwise specified, as the “optionally halogenated C₁₋₆ alkoxy” in the present specification, the above-mentioned “C₁₋₆ alkoxy” optionally substituted by 1 to 5 above-mentioned “halogen atoms” can be mentioned. For example, methoxy, ethoxy, isopropoxy, tert-butoxy, trifluoromethoxy and the like can be mentioned.

Unless otherwise specified, as the “mono- or di-C₁₋₆ alkyl-amino” in the present specification, amino mono- or di-substituted by the above-mentioned “C₁₋₆ alkyl” can be mentioned. For example, methylamino, ethylamino, propylamino, dimethylamino, diethylamino and the like can be mentioned.

Unless otherwise specified, as the “mono- or di-C₆₋₁₄ aryl-amino” in the present specification, amino mono- or di-substituted by the above-mentioned “C₆₋₁₄ aryl” can be mentioned. For example, phenylamino, diphenylamino, 1-naphthylamino, 2-naphthylamino and the like can be mentioned.

Unless otherwise specified, as the “mono- or di-C₇₋₁₆ aralkyl-amino” in the present specification, amino mono- or di-substituted by the above-mentioned “C₇₋₁₆ aralkyl” can be mentioned. For example, benzylamino, phenethylamino and the like can be mentioned.

Unless otherwise specified, as the “N—C₁₋₆ alkyl-N—C₆₋₁₄ aryl-amino” in the present specification, amino substituted by the above-mentioned “C₁₋₆ alkyl” and the above-mentioned “C₆₋₁₄ aryl” can be mentioned. For example, N-methyl-N-phenylamino, N-ethyl-N-phenylamino and the like can be mentioned.

Unless otherwise specified, as the “N—C₁₋₆ alkyl-N—C₇₋₁₆ aralkyl-amino” in the present specification, amino substituted by the above-mentioned “C₁₋₆ alkyl” and the above-mentioned “C₇₋₁₆ aralkyl” can be mentioned. For example, N-methyl-N-benzylamino, N-ethyl-N-benzylamino and the like can be mentioned.

Unless otherwise specified, as the “mono- or di-C₁₋₆ alkyl-carbamoyl” in the present specification, carbamoyl mono- or di-substituted by the above-mentioned “C₁₋₆ alkyl group” can be mentioned. For example, methylcarbamoyl, ethylcarbamoyl, dimethylcarbamoyl, diethylcarbamoyl, ethylmethylcarbamoyl and the like can be mentioned.

Unless otherwise specified, as the “mono- or di-C₆₋₁₄ aryl-carbamoyl” in the present specification, carbamoyl mono- or di-substituted by the above-mentioned “C₆₋₁₄ aryl” can be mentioned. For example, phenylcarbamoyl, 1-naphthylcarbamoyl, 2-naphthylcarbamoyl and the like can be mentioned.

Unless otherwise specified, as the “mono- or di-C₃₋₈ cycloalkyl-carbamoyl” in the present specification, carbamoyl mono- or di-substituted by the above-mentioned “C₃₋₈cycloalkyl” can be mentioned. For example, cyclopropylcarbamoyl and the like can be mentioned.

Unless otherwise specified, as the “mono- or di-C₇₋₁₆ aralkyl-carbamoyl”, carbamoyl mono- or di-substituted by the above-mentioned “C₇₋₁₆ aralkyl” can be mentioned. For example, benzylcarbamoyl and the like can be mentioned.

Unless otherwise specified, as the “mono- or di-5- to 7-membered heterocyclyl-carbamoyl” in the present specification, carbamoyl mono- or di-substituted by 5- to 7-membered heterocyclic group can be mentioned. As the 5- to 7-membered heterocyclic group, a heterocyclic group containing, as a ring-constituting atom besides carbon atoms, one or two kinds of 1 to 4 hetero atoms selected from a nitrogen atom, a sulfur atom and an oxygen atom can be mentioned. As preferable examples of the “mono- or di-5 to 7-membered heterocyclyl-carbamoyl”, 2-pyridylcarbamoyl, 3-pyridylcarbamoyl, 4-pyridylcarbamoyl, 2-thienylcarbamoyl, 3-thienylcarbamoyl and the like can be mentioned.

Unless otherwise specified, as the “mono- or di-C₁₋₆ alkyl-sulfamoyl” in the present specification, sulfamoyl mono- or di-substituted by the above-mentioned “C₁₋₆ alkyl” can be used, for example, methylsulfamoyl, ethylsulfamoyl, dimethylsulfamoyl, diethylsulfamoyl and the like can be mentioned.

Unless otherwise specified, as the “mono- or di-C₆₋₁₄ aryl-sulfamoyl” in the present specification, sulfamoyl mono- or di-substituted by the above-mentioned “C₆₋₁₄ aryl” can be used, for example, phenylsulfamoyl, diphenylsulfamoyl, 1-naphthylsulfamoyl, 2-naphthylsulfamoyl and the like can be mentioned.

Unless otherwise specified, as the “mono- or di-C₇₋₁₆ aralkyl-sulfamoyl” in the present specification, sulfamoyl mono- or di-substituted by the above-mentioned “C₇₋₁₆ aralkyl” can be mentioned, for example, benzylsulfamoyl and the like can be mentioned.

Examples of the “optionally substituted C₁₋₆ alkyl”, “optionally substituted C₂₋₆alkenyl”, “optionally substituted C₂₋₆alkynyl”, “optionally substituted C₁₋₆ alkoxy” and “optionally substituted C₁₋₆ alkylthio” in the present specification include “C₁₋₆ alkyl”, “C₂₋₆alkenyl”, “C₂₋₆alkynyl”, “C₁₋₆ alkoxy” and “C₁₋₆ alkylthio”, each of which optionally has, at substitutable position(s), 1 to 5 substituents selected from

(1) a halogen atom;
(2) hydroxy;
(3) amino;
(4) nitro;
(5) cyano;
(6) a heterocyclic group (preferably furyl, pyridyl, thienyl, pyrazolyl, thiazolyl, oxazolyl) optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈ cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl;
(7) mono- or di-C₁₋₆ alkyl-amino;
(8) mono- or di-C₆₋₁₄ aryl-amino;
(9) mono- or di-C₇₋₁₆ aralkyl-amino;
(10) N—C₁₋₆ alkyl-N—C₆₋₁₄ aryl-amino;
(11) N—C₁₋₆ alkyl-N—C₇₋₁₆ aralkyl-amino;
(12) C₃₋₈cycloalkyl;
(13) optionally halogenated C₁₋₆ alkoxy;
(14) C₁₋₆ alkylthio;
(15) C₁₋₆ alkylsulfinyl;
(16) C₁₋₆ alkylsulfonyl;
(17) optionally esterified carboxyl;
(18) C₁₋₆ alkyl-carbonyl;
(19) C₃₋₈ cycloalkyl-carbonyl;
(20) C₆₋₁₄ aryl-carbonyl;
(21) carbamoyl;
(22) thiocarbamoyl;
(23) mono- or di-C₁₋₆ alkyl-carbamoyl;
(24) mono- or di-C₆₋₁₄ aryl-carbamoyl;
(25) mono- or di-5- to 7-membered heterocyclyl-carbamoyl;
(26) C₁₋₆ alkyl-carbonylamino (e.g., acetylamino, propionylamino) optionally substituted by carboxyl;
(27) C₆₋₁₄ aryloxy optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl;
(28) C₆₋₁₄ aryl optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl;
(29) heterocyclyl-oxy;
(30) sulfamoyl;
(31) mono- or di-C₁₋₆ alkyl-sulfamoyl;
(32) mono- or di-C₆₋₁₄ aryl-sulfamoyl;
(33) C₇₋₁₆ aralkyloxy optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈ cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl; and the like. When the number of the substituents is plural, the respective substituents may be the same or different.

Examples of the “optionally substituted C₃₋₈cycloalkyl”, “optionally substituted C₆₋₁₄ aryl”, “optionally substituted C₇₋₁₆ aralkyl”, “optionally substituted heterocyclic group”, “optionally substituted heterocyclyl-oxy”, “optionally substituted C₆₋₁₄ aryloxy”, “optionally substituted C₇₋₁₆ aralkyloxy”, “optionally substituted heterocyclyl-thio”, “optionally substituted C₆₋₁₄ arylthio” and “optionally substituted C₇₋₁₆ aralkylthio” in the present specification include, “C₃₋₈ cycloalkyl”, “C₆₋₁₄ aryl”, “C₇₋₁₆ aralkyl”, “heterocyclic group”, “heterocyclyl-oxy”, “C₆₋₁₄ aryloxy”, “C₇₋₁₆ aralkyloxy”, “heterocyclyl-thio”, “C₆₋₁₄ arylthio” and “C₇₋₁₆ aralkylthio”, which optionally have, at substitutable position(s), 1 to 5 substituents selected from

(1) a halogen atom;
(2) hydroxy;
(3) amino;
(4) nitro;
(5) cyano;
(6) optionally substituted C₁₋₆ alkyl;
(7) optionally substituted C₂₋₆ alkenyl;
(8) optionally substituted C₂₋₆alkynyl;
(9) C₆₋₁₄ aryl optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈ cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl;
(10) C₆₋₁₄ aryloxy optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈ cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl;
(11) C₇₋₁₆ aralkyloxy optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈ cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl;
(12) a heterocyclic group (preferably furyl, pyridyl, thienyl, pyrazolyl, thiazolyl, oxazolyl) optionally substituted by 1 to 3 substituents selected from a halogen atom, hydroxy, amino, nitro, cyano, optionally halogenated C₁₋₆ alkyl, mono- or di-C₁₋₆ alkyl-amino, C₆₋₁₄ aryl, mono- or di-C₆₋₁₄ aryl-amino, C₃₋₈ cycloalkyl, C₁₋₆ alkoxy, C₁₋₆ alkoxy-C₁₋₆ alkoxy, C₁₋₆ alkylthio, C₁₋₆ alkylsulfinyl, C₁₋₆ alkylsulfonyl, optionally esterified carboxyl, carbamoyl, thiocarbamoyl, mono- or di-C₁₋₆ alkyl-carbamoyl, mono- or di-C₆₋₁₄ aryl-carbamoyl, sulfamoyl, mono- or di-C₁₋₆ alkyl-sulfamoyl and mono- or di-C₆₋₁₄ aryl-sulfamoyl;
(13) mono- or di-C₁₋₆ alkyl-amino;
(14) mono- or di-C₆₋₁₄ aryl-amino;
(15) mono- or di-C₇₋₁₆ aralkyl-amino;
(16) N—C₁₋₆ alkyl-N—C₆₋₁₄ aryl-amino;
(17) N—C₁₋₆ alkyl-N—C₇₋₁₆ aralkyl-amino;
(18) C₃₋₈cycloalkyl;
(19) optionally substituted C₁₋₆ alkoxy;
(20) optionally substituted C₁₋₆ alkylthio;
(21) C₁₋₆ alkylsulfinyl;
(22) C₁₋₆ alkylsulfonyl;
(23) optionally esterified carboxyl;
(24) C₁₋₆ alkyl-carbonyl;
(25) C₃₋₈cycloalkyl-carbonyl;
(26) C₆₋₁₄ aryl-carbonyl;
(27) carbamoyl;
(28) thiocarbamoyl;
(29) mono- or di-C₁₋₆ alkyl-carbamoyl;
(30) mono- or di-C₆₋₁₄ aryl-carbamoyl;
(31) mono- or di-5- to 7-membered heterocyclyl-carbamoyl;
(32) sulfamoyl;
(33) mono- or di-C₁₋₆ alkyl-sulfamoyl;
(34) mono- or di-C₆₋₁₄ aryl-sulfamoyl;
(35) C₁₋₆ alkyl-carbonylamino (e.g.: acetylamino, propionylamino) optionally substituted by carboxyl;
(36) heterocyclyl-oxy; and the like. When the number of the substituents is plural, the respective substituents may be the same or different.

Unless otherwise specified, as the “optionally substituted amino” in the present specification, an amino optionally substituted by 1 or 2 substituents selected from

(1) optionally substituted C₁₋₆ alkyl;
(2) optionally substituted C₂₋₆ alkenyl;
(3) optionally substituted C₂₋₆alkynyl;
(4) optionally substituted C₃₋₈ cycloalkyl;
(5) optionally substituted C₆₋₁₄ aryl;
(6) optionally substituted C₁₋₆ alkoxy;
(7) optionally substituted acyl;
(8) an optionally substituted heterocyclic group (preferably furyl, pyridyl, thienyl, pyrazolyl, thiazolyl, oxazolyl);
(9) sulfamoyl;
(10) mono- or di-C₁₋₆ alkyl-sulfamoyl;
(11) mono- or di-C₆₋₁₄ aryl-sulfamoyl; and the like, can be mentioned. When the “optionally substituted amino” is amino substituted by two substituents, these substituents may be the same or different, and may form a nitrogen-containing heterocycle together with the adjacent nitrogen atom. As the “nitrogen-containing heterocycle”, for example, a 5- to 7-membered nitrogen-containing heterocycle containing, as a ring-constituting atom besides carbon atoms, at least one nitrogen atom and optionally further containing 1 or 2 hetero atoms selected from an oxygen atom, a sulfur atom and a nitrogen atom can be mentioned. As preferable examples of the nitrogen-containing heterocycle, pyrrolidine, imidazolidine, pyrazolidine, piperidine, piperazine, morpholine, thiomorpholine, thiazolidine, oxazolidine and the like can be mentioned.

Unless otherwise specified, as the “optionally substituted acyl” in the present specification, groups represented by the formulas: —COR⁷, —CO—OR⁷, —SO₂R⁷, —SOR⁷, —PO(OR⁷)(OR⁸), —CO—NR^{7a}R^{8a} and —CS—NR^{7a}R^{8a}, wherein R⁷ and R⁸ are the same or different and each is a hydrogen atom, an optionally substituted hydrocarbon group or an optionally substituted heterocyclic group, and R^{7a} and R^{8a} are the same or different and each is a hydrogen atom, an optionally substituted hydrocarbon group or an optionally substituted heterocyclic group, or R^{7a} and R^{8a} may form an optionally substituted nitrogen-containing heterocycle together with the adjacent nitrogen atom, and the like can be mentioned.

As the “nitrogen-containing heterocycle” of the “optionally substituted nitrogen-containing heterocycle” formed by R^{7a} and R^{8a} together with the adjacent nitrogen atom, for example, a 5- to 7-membered nitrogen-containing heterocycle containing, as a ring-constituting atom besides carbon atom, at least one nitrogen atom and optionally further containing 1 to 2 hetero atoms selected from an oxygen atom, a sulfur atom and a nitrogen atom can be mentioned. As preferable examples of the “nitrogen-containing heterocycle”, pyrrolidine, imidazolidine, pyrazolidine, piperidine, piperazine, morpholine, thiomorpholine, thiazolidine, oxazolidine and the like can be mentioned.

The nitrogen-containing heterocycle optionally has 1 to 2 substituents at substitutable position(s). As these substituents, hydroxy, optionally halogenated C₁₋₆ alkyl, C₆₋₁₄ aryl, C₇₋₁₆ aralkyl and the like can be mentioned. When the number of the substituents is two, these substituents may be the same or different.

Preferable examples of the “optionally substituted acyl” include

formyl;
carboxyl;
carbamoyl;
C₁₋₆ alkyl-carbonyl;
C₁₋₆ alkoxy-carbonyl;
C₃₋₈ cycloalkyl-carbonyl;
C₆₋₁₄ aryl-carbonyl;
C₇₋₁₆ aralkyl-carbonyl;
C₆₋₁₄ aryloxy-carbonyl;
C₇₋₁₆ aralkyloxy-carbonyl;
mono- or di-C₁₋₆ alkyl-carbamoyl;
mono- or di-C₆₋₁₄ aryl-carbamoyl;
mono- or di-C₃₋₈cycloalkyl-carbamoyl;
mono- or di-C₇₋₁₆ aralkyl-carbamoyl;
C₁₋₆ alkylsulfonyl;
C₆₋₁₄ arylsulfonyl optionally substituted by nitro;
nitrogen-containing heterocyclyl-carbonyl;
C₁₋₆ alkylsulfinyl;
C₆₋₁₄ arylsulfinyl;
thiocarbamoyl;
sulfamoyl;
mono- or di-C₁₋₆ alkyl-sulfamoyl;
mono- or di-C₆₋₁₄ aryl-sulfamoyl;
mono- or di-C₇₋₁₆ aralkyl-sulfamoyl;
and the like.

The definition of each symbol in the formula (I) and (II) is explained in detail in the following.

R¹ shows a halogen atom, hydroxy, optionally substituted C₁₋₆ alkyl or optionally substituted C₁₋₆ alkoxy.

The “optionally substituted C₁₋₆ alkyl” or “optionally substituted C₁₋₆ alkoxy” for R¹ is optionally substituted by 1 to 3 substituents selected from those exemplified as the substituents of the aforementioned “optionally substituted C₁₋₆ alkyl”, “optionally substituted C₂₋₆alkenyl”, “optionally substituted C₂₋₆alkynyl”, “optionally substituted C₁₋₆ alkoxy” and “optionally substituted C₁₋₆ alkylthio”. When the number of the substituents is plural, the respective substituents may be the same or different.

In addition, when R¹ is optionally substituted C₁₋₆ alkyl or optionally substituted C₁₋₆ alkoxy, R¹ may form an optionally substituted ring together with X. Examples of R¹ and X forming a ring include

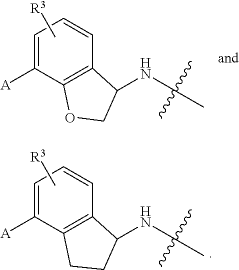

R¹ is preferably C₁₋₆ alkyl or C₁₋₆ alkoxy which may form an optionally substituted ring together with X, more preferably, methyl which may form an optionally substituted ring together with X, ethyl which may form an optionally substituted ring together with X or methoxy which may form an optionally substituted ring together with X. R¹ particularly preferably forms

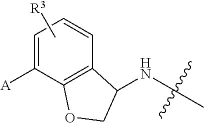

together with X. In other words, in a preferable embodiment, compound (I) is a compound represented by the formula (II):

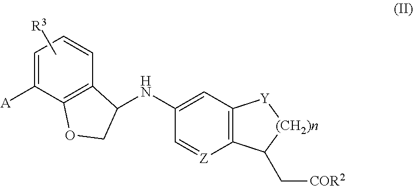

(compound (II)).

R² is optionally substituted hydroxy.

The “optionally substituted hydroxy” for R² is hydroxy optionally substituted by substituent(s) selected from C₁₋₆ alkyl, heterocyclyl-oxy, C₆₋₁₄ aryloxy and C₇₋₁₆ aralkyloxy.

R² is preferably hydroxy.

R³ is a hydrogen atom, a halogen atom or optionally substituted C₁₋₆ alkyl.

The “optionally substituted C₁₋₆ alkyl” for R³ is optionally substituted by 1 to 3 substituents selected from those recited above as the substituents of “optionally substituted C₁₋₆ alkyl”, “optionally substituted C₂₋₆alkenyl”, “optionally substituted C₂₋₆alkynyl”, “optionally substituted C₁₋₆ alkoxy” and “optionally substituted C₁₋₆ alkylthio”. As such substituent, preferred is a halogen atom. When the number of the substituents is plural, the respective substituents may be the same or different.

R³ is preferably a hydrogen atom or C₁₋₆ alkyl.

X is CH₂. Here, X may form an optionally substituted ring together with R¹, and examples of such ring include those mentioned above.

X preferably forms

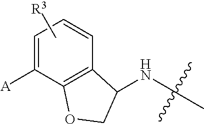

together with R¹.

Y is CH₂, NH or O.

Y is preferably O.

Z is CH or N.

Z is preferably CH.

n is an integer selected from 1 to 3.

n is preferably 1.

A is a halogen atom, optionally substituted amino, or a 4- to 13-membered cyclic group optionally substituted by 1 to 5 (preferably 1 to 3) substituents selected from

(1) a halogen atom,
(2) optionally substituted amino,
(3) optionally substituted C₁₋₆ alkylthio,
(4) optionally substituted C₁₋₆ alkyl,
(5) optionally substituted C₃₋₁₀ cycloalkyl,
(6) optionally substituted C₁₋₆ alkoxy,
(7) optionally substituted C₆₋₁₄ aryl,
(8) an optionally substituted 4- to 7-membered heterocyclic group, and
(9) optionally substituted 4- to 7-membered heterocyclyl-oxy. When the number of the substituents is plural, the respective substituents may be the same or different.

Examples of the “4- to 13-membered cyclic group” for A include a 4- to 13-membered cyclic hydrocarbon group and a 4- to 13-membered heterocyclic group.

Examples of the “cyclic hydrocarbon group” include an alicyclic hydrocarbon group comprised of 4 to 13 carbon atoms, an aromatic hydrocarbon group comprised of 6 to 14 carbon atoms and the like.

Examples of the “alicyclic hydrocarbon group” include C₃₋₆ cycloalkyl (e.g., cyclopropyl, cyclobutyl, cyclopentyl, cyclohexyl), C₃₋₆cycloalkenyl (e.g., cyclopentenyl, cyclohexenyl), C₅₋₁₄ cycloalkadienyl (e.g., 2,4-cyclopentadienyl, 1,3-cyclohexadienyl), indanyl, adamantyl and the like.

Examples of the “aromatic hydrocarbon group” include C₆₋₁₄ aryl (e.g., phenyl, naphthyl, anthracenyl, phenanthrenyl) and the like. Preferred is phenyl.

Examples of the “heterocyclic group” include those mentioned above. Preferred are pyridyl, pyrimidinyl, piperidinyl, pyrazolyl, thienyl, morpholinyl, dihydropyranyl, 1,4-dioxaspiro[4.5]dec-7-enyl, 1,4-dioxa-8-azaspiro[4.5]decanyl, benzimidazolyl, 3,4-dihydro-2H-pyrido[3,2-b] [1,4]oxazinyl, imidazo[2,3-a]pyridyl, imidazo[1,2-a]pyridyl, furo[2,3-b]pyridyl, furo[3,2-b]pyridyl, indolyl and 3,4-dihydro-2H-1,4-benzooxazinyl.

The “optionally substituted amino” for A and the “optionally substituted amino” shown as the substituent of the “4- to 13-membered cyclic group” for A are each optionally substituted by 1 or 2 substituents selected from those exemplified as the substituent of the aforementioned “optionally substituted C₁₋₆ alkyl”, “optionally substituted C₂₋₆ alkenyl”, “optionally substituted C₂₋₆alkynyl”, “optionally substituted C₁₋₆ alkoxy” and “optionally substituted C₁₋₆ alkylthio”. When the number of the substituents is plural, the respective substituents may be the same or different.

The “optionally substituted C₁₋₆ alkylthio”, “optionally substituted C₁₋₆ alkyl” and “optionally substituted C₁₋₆ alkoxy” shown as the substituents of the “4- to 13-membered cyclic group” for A is optionally substituted by 1 to 3 substituents selected from those exemplified as the substituents of the aforementioned “optionally substituted C₁₋₆ alkyl”, “optionally substituted C₂₋₆alkenyl”, “optionally substituted C₂₋₆alkynyl”, “optionally substituted C₁₋₆ alkoxy” and “optionally substituted C₁₋₆ alkylthio”. When the number of the substituents is plural, the respective substituents may be the same or different.

The “optionally substituted C₃₋₁₀ cycloalkyl”, “optionally substituted C₆₋₁₄ aryl”, “optionally substituted 4- to 7-membered heterocyclic group” and “optionally substituted 4- to 7-membered heterocyclyl-oxy” shown as the substituents of the “4- to 13-membered cyclic group” for A is optionally substituted by 1 to 3 substituents selected from those exemplified as the substituents of the aforementioned “optionally substituted C₃₋₈cycloalkyl”, “optionally substituted C₆₋₁₄ aryl”, “optionally substituted C₇₋₁₆ aralkyl”, “optionally substituted heterocyclic group”, “optionally substituted heterocyclyl-oxy”, “optionally substituted C₆₋₁₄ aryloxy”, “optionally substituted C₇₋₁₆ aralkyloxy”, “optionally substituted heterocyclyl-thio”, “optionally substituted C₆₋₁₄ arylthio” and “optionally substituted C₇₋₁₆ aralkylthio”. When the number of the substituents is plural, the respective substituents may be the same or different.

A is preferably a 4- to 13-membered cyclic group optionally substituted by 1 to 5 (preferably, 1 to 3) substituents selected from

(1) a halogen atom,
(2) optionally substituted C₁₋₆ alkyl (preferably, optionally substituted by a halogen atom or hydroxy),
(3) optionally substituted C₁₋₆ alkoxy (preferably, optionally substituted by a halogen atom, C₁₋₆ alkylsulfonyl, C₆₋₁₄ aryl or a heterocyclic group),
(4) an optionally substituted 4- to 7-membered heterocyclic group, and
(5) optionally substituted 4- to 7-membered heterocyclyl-oxy.

A is more preferably phenyl or a 5- or 6-membered aromatic heterocyclic group (e.g., pyrazolyl, thienyl, pyrimidinyl), each of which is optionally substituted by 1 to 5 (preferably, 1 to 3) substituents selected from

(a) a halogen atom,
(b) C₁₋₆ alkyl (e.g., methyl) optionally substituted by 1 to 3 halogen atoms (e.g., a fluorine atom),
(c) C₁₋₆ alkoxy (e.g., methoxy, ethoxy, propoxy) optionally substituted by 1 to 3 substituents selected from
(1) a halogen atom,
(2) C₁₋₆ alkylsulfonyl (e.g., methylsulfonyl),
(3) C₃₋₈cycloalkyl (e.g., cyclopropyl, cyclopentyl),
(4) mono- or di-C₁₋₆ alkyl-amino (e.g., dimethylamino),
(5) C₁₋₆ alkoxy (e.g., methoxy),
(6) C₆₋₁₄ aryl (e.g., phenyl) optionally substituted by a halogen atom (e.g., a fluorine atom),
(7) a heterocyclic group (e.g., furyl, isoxazolyl, pyridyl, pyrrolidinyl, morpholinyl, piperazinyl, azepanyl) optionally substituted by 1 to 3 substituents selected from
   (i) C₁₋₆ alkyl (e.g., methyl), and
   (ii) oxo, and
(8) C₁₋₆ alkylthio (e.g., methylthio),
   (d) a 4- to 7-membered heterocyclic group (e.g., morpholino, pyrrolidinyl), and
   (e) 4- to 7-membered heterocyclyl-oxy (e.g., dioxidotetrahydrothiopyranyloxy).

In another preferable embodiment, A is preferably

(a) a halogen atom (e.g., bromine atom),
(b) amino optionally substituted by 1 or 2 substituents selected from
(i) C₁₋₆ alkyl (e.g., ethyl, n-propyl),
(ii) C₆₋₁₄ aryl (e.g., phenyl) optionally substituted by substituent(s) selected from a halogen atom (e.g., a fluorine atom), cyano and C₁₋₆ alkylsulfonyl (e.g., mesyl), and
(iii) a heterocyclic group (e.g., pyridyl, pyrimidinyl) optionally substituted by substituent(s) selected from a halogen atom (e.g., a fluorine atom, a chlorine atom) and C₁₋₆ alkyl (e.g., methyl), or
   (c) a 4- to 13-membered cyclic group (e.g., phenyl, pyrazolyl, thienyl, piperidinyl, morpholinyl, pyridyl, pyrimidinyl, dihydropyranyl, indolyl, benzimidazolyl, furo[2,3-b]pyridinyl, furo[3,2-b]pyridinyl, imidazo[1,2-a]pyridinyl, 3,4-dihydro-2H-1,4-benzoxazinyl, 3,4-dihydro-2H-pyrido[3,2-b] [1,4]oxazinyl, 1,4-dioxaspiro[4.5]dec-7-enyl, 1,4-dioxa-8-azaspiro[4.5]decanyl) optionally substituted by 1 to 5 (preferably, 1 to 3) substituents selected from
(1) a halogen atom (e.g., a fluorine atom, a chlorine atom),
(2) optionally substituted amino,
(3) C₁₋₆ alkylthio (e.g., methylthio) optionally substituted by C₆₋₁₄ aryl (e.g., phenyl) optionally substituted by a halogen atom (e.g., a chlorine atom),
(4) C₁₋₆ alkyl (e.g., methyl, ethyl, isopropyl) optionally substituted by substituent(s) selected from
   (i) a halogen atom (e.g., a fluorine atom),
   (ii) optionally substituted hydroxy (e.g., hydroxy, methoxy), and
   (iii) C₁₋₆ alkylsulfonyl (e.g., mesyl),
(5) optionally substituted C₃₋₁₀ cycloalkyl (e.g., cyclopropyl),
(6) C₁₋₆ alkoxy (e.g., methoxy, ethoxy, n-propoxy) optionally substituted by substituent(s) selected from
   (i) a halogen atom (e.g., a fluorine atom),
   (ii) C₃₋₈ cycloalkyl (e.g., cyclopropyl, cyclobutyl, cyclopentyl) optionally substituted by C₁₋₆ alkyl (e.g., methyl),
   (iii) C₁₋₆ alkoxy (e.g., methoxy, ethoxy) optionally substituted by di-C₁₋₆ alkylamino (e.g., dimethylamino),
   (iv) di-C₁₋₆ alkylamino (e.g., dimethylamino),
   (v) C₁₋₆ alkylsulfinyl (e.g., methylsulfinyl),
   (vi) mesyl,
   (vii) C₆₋₁₄ aryl (e.g., phenyl) optionally substituted by a halogen atom (e.g., a fluorine atom), and
   (viii) a heterocyclic group (e.g., furyl, isoxazolyl, pyridyl, pyrrolidinyl, morpholinyl, piperazinyl, azepanyl) optionally substituted by substituent(s) selected from C₁₋₆ alkyl (e.g., methyl) and oxo,
   (7) optionally substituted C₆₋₁₄ aryl,
   (8) a 4- to 7-membered heterocyclic group (e.g., tetrahydrofuranyl, morpholino, pyrrolidinyl, tetrahydropyranyl, furanyl, isoxazolyl) optionally substituted by C₁₋₆ alkyl (e.g., methyl), and
   (9) optionally substituted 4- to 7-membered heterocyclyl-oxy (e.g., dioxidotetrahydrothiopyranyloxy).

A is more preferably phenyl, benzimidazolyl or a 5- or 6-membered aromatic heterocyclic group (e.g., thienyl, pyrimidinyl) each optionally substituted by 1 to 5 (preferably, 1 to 3) substituents selected from

(a) a halogen atom,
(b) C₁₋₆ alkyl (e.g., methyl) optionally substituted by 1 to 3 halogen atoms (e.g., a fluorine atom),
(c) C₁₋₆ alkoxy (e.g., methoxy, ethoxy, propoxy) optionally substituted by 1 to 3 substituents selected from
(1) a halogen atom,
(2) C₁₋₆ alkylsulfonyl (e.g., methylsulfonyl),
(3) C₃₋₈cycloalkyl (e.g., cyclopropyl, cyclopentyl),
(4) mono- or di-C₁₋₆ alkyl-amino (e.g., dimethylamino),
(5) C₁₋₆ alkoxy (e.g., methoxy),
(6) C₆₋₁₄ aryl (e.g., phenyl) optionally substituted by a halogen atom (e.g., a fluorine atom),
(7) a 4- to 7-membered heterocyclic group (e.g., furyl, isoxazolyl, pyridyl, pyrrolidinyl, morpholinyl, piperazinyl, azepanyl) optionally substituted by 1 to 3 substituents selected from C₁₋₆ alkyl (e.g., methyl) and oxo, and
(8) C₁₋₆ alkylthio (e.g., methylthio),
   (d) a 4- to 7-membered heterocyclic group (e.g., morpholino, pyrrolidinyl), and
   (e) 4- to 7-membered heterocyclyl-oxy (e.g., dioxidotetrahydrothiopyranyloxy) optionally substituted by 1 to 3 substituents selected from C₁₋₆ alkyl and oxo.

A is more preferably phenyl optionally substituted by 1 to 3 substituents selected from

(a) C₁₋₆ alkyl (e.g., methyl) optionally substituted by 1 to 3 halogen atoms (e.g., a fluorine atom),
(b) C₁₋₆ alkoxy (e.g., methoxy, ethoxy, propoxy) optionally substituted by 1 to 3 substituents selected from
(1) a halogen atom,
(2) C₁₋₆ alkylsulfonyl (e.g., methylsulfonyl),
(3) C₃₋₈cycloalkyl (e.g., cyclopropyl, cyclopentyl),
(4) mono- or di-C₁₋₆ alkyl-amino (e.g., dimethylamino),
(5) C₁₋₆ alkoxy (e.g., methoxy),
(6) C₆₋₁₄ aryl (e.g., phenyl) optionally substituted by a halogen atom (e.g., a fluorine atom),
(7) a 4- to 7-membered heterocyclic group (e.g., furyl, isoxazolyl, pyridyl, pyrrolidinyl, morpholinyl, piperazinyl, azepanyl) optionally substituted by 1 to 3 substituents selected from C₁₋₆ alkyl (e.g., methyl) and oxo, and
(8) C₁₋₆ alkylthio (e.g., methylthio), and
   (c) 4- to 7-membered heterocyclyl-oxy (e.g., dioxidotetrahydrothiopyranyloxy) optionally substituted by 1 to 3 substituents selected from C₁₋₆ alkyl and oxo.

Alternatively, A is more preferably benzimidazolyl or a 5- or 6-membered aromatic heterocyclic group (e.g., thienyl, pyrimidinyl) each optionally substituted by 1 to 3 substituents selected from

(a) a halogen atom,
(b) C₁₋₆ alkyl (e.g., methyl) optionally substituted by 1 to 3 halogen atoms (e.g., a fluorine atom), and
(c) a 5- or 6-membered heterocyclic group (e.g., morpholino, pyrrolidinyl).

Preferable examples of compound (I) include the following compounds.

### [Compound A-1]

Compound (I) wherein
R¹ is C₁₋₆ alkyl,
R² is hydroxy,
R³ is a hydrogen atom or C₁₋₆ alkyl,

### X is CH₂,

### Y is O,

### Z is CH,

n is 1, and
A is a 4- to 13-membered cyclic group optionally substituted by 1 to 3 substituents selected from
(1) a halogen atom,
(2) optionally substituted C₁₋₆ alkyl (optionally substituted by a halogen atom or hydroxy),
(3) optionally substituted C₁₋₆ alkoxy (optionally substituted by a halogen atom, mesyl, aryl or a heterocyclic group),
(4) an optionally substituted 4- to 7-membered heterocyclic group, and
(5) optionally substituted 4- to 7-membered heterocyclyl-oxy.

### [Compound A-2]

Compound (I) wherein
R¹ is C₁₋₆ alkyl (e.g., methyl),
R² is hydroxy,
R³ is a hydrogen atom or C₁₋₆ alkyl (e.g., methyl) optionally substituted by 1 to 3 halogen atoms (e.g., a fluorine atom),

### X is CH₂,

### Y is O,

### Z is CH,

n is 1, and

### A is

(a) a halogen atom (e.g., a bromine atom),
(b) amino optionally substituted by 1 or 2 substituents selected from
(i) C₁₋₆ alkyl (e.g., n-propyl), and
(ii) a heterocyclic group (e.g., pyridyl) optionally substituted by a halogen atom (e.g., a fluorine atom), or
   (c) a 4- to 13-membered cyclic group (e.g., phenyl, pyrazolyl, thienyl, piperidinyl, pyridyl, pyrimidinyl, dihydropyranyl, indolyl, benzimidazolyl, furo[2,3-b]pyridinyl, furo[3,2-b]pyridinyl, imidazo[1,2-a]pyridinyl, 3,4-dihydro-2H-pyrido[3,2-b] [1,4]oxazinyl, 1,4-dioxaspiro[4.5]dec-7-enyl, 1,4-dioxa-8-azaspiro[4.5]decanyl) optionally substituted by 1 to 3 substituents selected from
(1) a halogen atom (e.g., a fluorine atom, a chlorine atom),
(2) optionally substituted amino,
(3) C₁₋₆ alkylthio (e.g., methylthio) optionally substituted by C₆₋₁₄ aryl (e.g., phenyl) optionally substituted by a halogen atom (e.g., a chlorine atom),
(4) C₁₋₆ alkyl (e.g., methyl, ethyl, isopropyl) optionally substituted by substituent(s) selected from
   (i) a halogen atom (e.g., a fluorine atom),
   (ii) optionally substituted hydroxy (e.g., hydroxy, methoxy), and
   (iii) C₁₋₆ alkylsulfonyl (e.g., mesyl),
(5) optionally substituted C₃₋₁₀ cycloalkyl (e.g., cyclopropyl),
(6) C₁₋₆ alkoxy (e.g., methoxy, ethoxy, n-propoxy) optionally substituted by substituent(s) selected from
   (i) a halogen atom (e.g., a fluorine atom),
   (ii) mesyl,
   (iii) C₆₋₁₄ aryl (e.g., phenyl), and
   (iv) a heterocyclic group,
(7) optionally substituted C₆₋₁₄ aryl,
(8) an optionally substituted 4- to 7-membered heterocyclic group (e.g., tetrahydrofuryl, morpholino), and
(9) optionally substituted 4- to 7-membered heterocyclyl-oxy.

### [Compound B-1]

Compound (I) wherein
R¹ forms

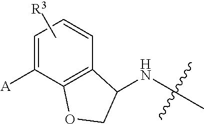

together with X (that is, compound (II)),
R² is hydroxy,
R³ is a hydrogen atom or C₁₋₆ alkyl,

### Y is O,

### Z is CH,

n is 1, and
A is a 4- to 13-membered cyclic group optionally substituted by 1 to 3 substituents selected from
(1) a halogen atom,
(2) optionally substituted C₁₋₆ alkyl (optionally substituted by a halogen atom or hydroxy),
(3) optionally substituted C₁₋₆ alkoxy (optionally substituted by a halogen atom, mesyl, aryl or a heterocyclic group),
(4) an optionally substituted 4- to 7-membered heterocyclic group, and
(5) optionally substituted 4- to 7-membered heterocyclyl-oxy.

### [Compound B-2]

Compound (I) wherein
R¹ forms

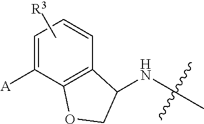

together with X (that is, compound (II)),
R² is hydroxy,
R³ is a hydrogen atom,

### Y is O,

### Z is CH,

n is 1, and

### A is

(a) a halogen atom (e.g., a bromine atom),
(b) amino optionally substituted by 1 or 2 substituents selected from
(i) C₁₋₆ alkyl (e.g., ethyl, n-propyl),
(ii) C₆₋₁₄ aryl (e.g., phenyl) optionally substituted by substituent(s) selected from a halogen atom (e.g., a fluorine atom), cyano and C₁₋₆ alkylsulfonyl (e.g., mesyl), and
(iii) a heterocyclic group (e.g., pyridyl, pyrimidinyl) optionally substituted by substituent(s) selected from a halogen atom (e.g., a fluorine atom, a chlorine atom) and C₁₋₆ alkyl (e.g., methyl), or
   (c) a 4- to 13-membered cyclic group (e.g., phenyl, pyrazolyl, thienyl, piperidinyl, morpholinyl, pyrimidinyl, benzimidazolyl, 3,4-dihydro-2H-1,4-benzoxazinyl) optionally substituted by 1 to 3 substituents selected from
(1) a halogen atom (e.g., a fluorine atom),
(2) optionally substituted amino,
(3) optionally substituted C₁₋₆ alkylthio,
(4) C₁₋₆ alkyl (e.g., methyl, ethyl, isopropyl) optionally substituted by a halogen atom (e.g., a fluorine atom),
(5) optionally substituted C₃₋₁₀ cycloalkyl,
(6) C₁₋₆ alkoxy (e.g., methoxy, ethoxy, n-propoxy) optionally substituted by substituent(s) selected from
   (i) a halogen atom (e.g., a fluorine atom),
   (ii) C₃₋₈cycloalkyl (e.g., cyclopropyl, cyclobutyl, cyclopentyl) optionally substituted by C₁₋₆ alkyl (e.g., methyl),
   (iii) C₁₋₆ alkoxy (e.g., methoxy, ethoxy) optionally substituted by di-C₁₋₆ alkylamino (e.g., dimethylamino),
   (iv) di-C₁₋₆ alkylamino (e.g., dimethylamino),
   (v) C₁₋₆ alkylsulfinyl (e.g., methylsulfinyl),
   (vi) mesyl,
   (vii) C₆₋₁₄ aryl (e.g., phenyl) optionally substituted by a halogen atom (e.g., a fluorine atom), and
   (viii) a heterocyclic group (e.g., furyl, isoxazolyl, pyridyl, pyrrolidinyl, morpholinyl, piperazinyl, azepanyl) optionally substituted by substituent(s) selected from C₁₋₆ alkyl (e.g., methyl) and oxo,
(7) optionally substituted C₆₋₁₄ aryl,
(8) a 4- to 7-membered heterocyclic group (e.g., morpholino, pyrrolidinyl, tetrahydropyranyl, furanyl, isoxazolyl) optionally substituted by C₁₋₆ alkyl (e.g., methyl), and
(9) optionally substituted 4- to 7-membered heterocyclyl-oxy (e.g., dioxidotetrahydrothiopyranyloxy).

### [Compound B-3]

Compound (I) wherein
R¹ forms

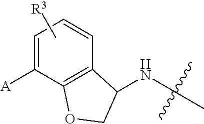

together with X (that is, compound (II)),
R² is hydroxy,
R³ is a hydrogen atom or C₁₋₆ alkyl,

### Y is O,

### Z is CH,

n is 1, and
A is phenyl, benzimidazolyl or a 5- or 6-membered aromatic heterocyclic group (preferably, thienyl, pyrimidinyl) each optionally substituted by 1 to 5 substituents selected from
(a) a halogen atom,
(b) C₁₋₆ alkyl optionally substituted by 1 to 3 halogen atoms,
(c) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio,
   (d) a 4- to 7-membered heterocyclic group, and
   (e) 4- to 7-membered heterocyclyl-oxy optionally substituted by 1 to 3 substituents selected from C₁₋₆ alkyl and oxo.

### [Compound B-4]

Compound (I) wherein
R¹ forms

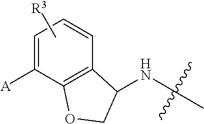

together with X (that is, compound (II)),
R² is hydroxy,
R³ is a hydrogen atom or C₁₋₆ alkyl,

### Y is O,

### Z is CH,

n is 1, and
A is phenyl optionally substituted by 1 to 3 substituents selected from
(a) C₁₋₆ alkyl optionally substituted by 1 to 3 halogen atoms,
(b) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio, and
   (c) 4- to 7-membered heterocyclyl-oxy optionally substituted by 1 to 3 substituents selected from C₁₋₆ alkyl and oxo.

### [Compound B-5]

Compound (I) wherein
R¹ forms

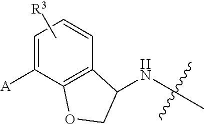

together with X (that is, compound (II)),
R² is hydroxy,
R³ is a hydrogen atom or C₁₋₆ alkyl,

### Y is O,

### Z is CH,

n is 1, and
A is benzimidazolyl or a 5- or 6-membered aromatic heterocyclic group (preferably, thienyl, pyrimidinyl) each optionally substituted by 1 to 3 substituents selected from
(a) a halogen atom,
(b) C₁₋₆ alkyl optionally substituted by 1 to 3 halogen atoms, and
(c) a 5- or 6-membered heterocyclic group.

### [Compound C-1]

Compound (I) wherein
R¹ forms

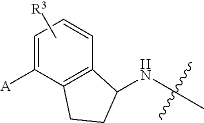

together with X,
R² is hydroxy,
R³ is a hydrogen atom or C₁₋₆ alkyl,

### Y is O,

### Z is CH,

n is 1,
A is a 4- to 13-membered cyclic group optionally substituted by 1 to 3 substituents selected from
(1) a halogen atom,
(2) optionally substituted (optionally substituted by a halogen atom or hydroxy) C₁₋₆ alkyl,
(3) optionally substituted (optionally substituted by a halogen atom, mesyl, aryl or a heterocyclic group) C₁₋₆ alkoxy,
(4) an optionally substituted 4- to 7-membered heterocyclic group, and
(5) optionally substituted 4- to 7-membered heterocyclyl-oxy.

### [Compound C-2]

R¹ forms

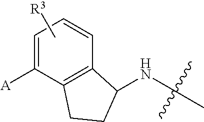

together with X,
R² is hydroxy,
R³ is a hydrogen atom or C₁₋₆ alkyl,

### Y is O,

### Z is CH, and

n is 1,

A is more preferably phenyl or a 5- or 6-membered aromatic heterocyclic group (e.g., pyrazolyl, thienyl, pyrimidinyl) each optionally substituted by 1 to 3 substituents selected from

(a) a halogen atom,
(b) C₁₋₆ alkyl (e.g., methyl) optionally substituted by 1 to 3 halogen atoms (e.g., a fluorine atom),
(c) C₁₋₆ alkoxy (e.g., methoxy, ethoxy, propoxy) optionally substituted by 1 to 3 substituents selected from
(1) a halogen atom,
(2) C₁₋₆ alkylsulfonyl (e.g., methylsulfonyl),
(3) C₃₋₈ cycloalkyl (e.g., cyclopropyl, cyclopentyl),
(4) mono- or di-C₁₋₆ alkyl-amino (e.g., dimethylamino),
(5) C₁₋₆ alkoxy (e.g., methoxy),
(6) C₆₋₁₄ aryl (e.g., phenyl) optionally substituted by a halogen atom (e.g., a fluorine atom),
(7) a heterocyclic group (e.g., furyl, isoxazolyl, pyridyl, pyrrolidinyl, morpholinyl, piperazinyl, azepanyl) optionally substituted by 1 to 3 substituents selected from
   (i) C₁₋₆ alkyl (e.g., methyl), and
   (ii) oxo, and
(8) C₁₋₆ alkylthio (e.g., methylthio),
   (d) a 4- to 7-membered heterocyclic group (e.g., morpholino, pyrrolidinyl), and
   (e) 4- to 7-membered heterocyclyl-oxy (e.g., dioxidotetrahydrothiopyranyloxy).

### [Compound D]

[(3S)-6-{[(3S)-7-{2,6-dimethyl-4-[3-(methylsulfonyl)propoxy]phenyl}-2,3-dihydro-1-benzofuran-3-yl]amino}-2,3-dihydro-1-benzofuran-3-yl]acetic acid or a salt thereof;
[(3S)-6-{[(3S)-7-{4-[(1,1-dioxidotetrahydro-2H-thiopyran-4-yl)oxy]-2,6-dimethylphenyl}-2,3-dihydro-1-benzofuran-3-yl]amino}-2,3-dihydro-1-benzofuran-3-yl]acetic acid or a salt thereof;
[(3S)-6-{[(3S)-7-(2-ethyl-6,7-difluoro-1H-benzimidazol-1-yl)-2,3-dihydro-1-benzofuran-3-yl]amino}-2,3-dihydro-1-benzofuran-3-yl]acetic acid or a salt thereof;
[(3S)-6-{[(3S)-7-(2-ethoxy-6,7-difluoro-1H-benzimidazol-1-yl)-2,3-dihydro-1-benzofuran-3-yl]amino}-2,3-dihydro-1-benzofuran-3-yl]acetic acid or a salt thereof;
[(3S)-6-({(3S)-7-[4,6-dimethyl-2-(morpholin-4-yl)pyrimidin-5-yl]-2,3-dihydro-1-benzofuran-3-yl}amino)-2,3-dihydro-1-benzofuran-3-yl]acetic acid or a salt thereof.

Examples of the salt of a compound represented by the formula (I) and (II) include metal salts, an ammonium salt, salts with organic bases, salts with inorganic acids, salts with organic acids, salts with basic or acidic amino acids and the like.

Preferable examples of the metal salt include alkali metal salts such as sodium salt, potassium salt and the like; alkaline earth metal salts such as calcium salt, magnesium salt, barium salt and the like; aluminum salt and the like.

Preferable examples of the salt with organic base include a salt with trimethylamine, triethylamine, pyridine, picoline, 2,6-lutidine, ethanolamine, diethanolamine, triethanolamine, cyclohexylamine, dicyclohexylamine, N,N-dibenzylethylenediamine and the like.

Preferable examples of the salt with inorganic acid include a salt with hydrochloric acid, hydrobromic acid, nitric acid, sulfuric acid, phosphoric acid and the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include a salt with arginine, lysin, ornithine and the like. Preferable examples of the salt with acidic amino acid include a salt with aspartic acid, glutamic acid and the like.

Among the above-mentioned salts, a pharmaceutically acceptable salt is preferable.

A prodrug of compound (I) is a compound that converts to compound (I) due to the reaction by enzyme, gastric acid and the like under the physiological conditions in the body; that is, a compound that converts to compound (I) by enzymatic oxidation, reduction, hydrolysis and the like, and a compound that converts to compound (I) by hydrolysis and the like by gastric acid and the like.

Examples of a prodrug of compound (I) include a compound wherein amino of compound (I) is acylated, alkylated or phosphorylated (e.g., compound wherein amino of compound (I) is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated); a compound wherein hydroxy of compound (I) is acylated, alkylated, phosphorylated or borated (e.g., a compound wherein hydroxy of compound (I) is acetylated, palmitoylated, propanoylated, pivaloylated, succinylated, fumarylated, alanylated or dimethylaminomethylcarbonylated); a compound wherein carboxyl of compound (I) is esterified or amidated (e.g., a compound wherein carboxyl of compound (I) is C₁₋₆ alkyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated) and the like. Of these, a compound wherein carboxyl of compound (I) is esterified by C₁₋₆ alkyl such as methyl, ethyl, tert-butyl and the like is preferably used. These compounds can be produced from compound (I) by a method known per se.

A prodrug of compound (I) may be a compound that converts to compound (I) under physiological conditions as described in Development of Pharmaceutical Products, vol. 7, Molecule Design, 163-198, Hirokawa Shoten (1990).

Hereinafter the production methods of the compound (I) or a salt thereof are explained.

Each symbol of the compounds in the schematic drawings of the following schemes is as defined above unless particularly described. Each compound described in the schemes may form a salt as long as it does not inhibit the reaction, and as such salt, those similar to the salts of compound (I) can be mentioned.

The compound obtained in each step can also be used as a crude product in the form of a reaction mixture in the next reaction, or can be isolated from the reaction mixture according to a conventional method, and further purified easily by a separation method such as recrystallization, distillation, chromatography and the like.

Compound (I) (e.g., compounds represented by the formulas (1a) and (1b) (to be abbreviated as compound (1a) and compound (1b), respectively)) can be produced, for example, according to the method as shown in the following Scheme 1 or a method analogous thereto.

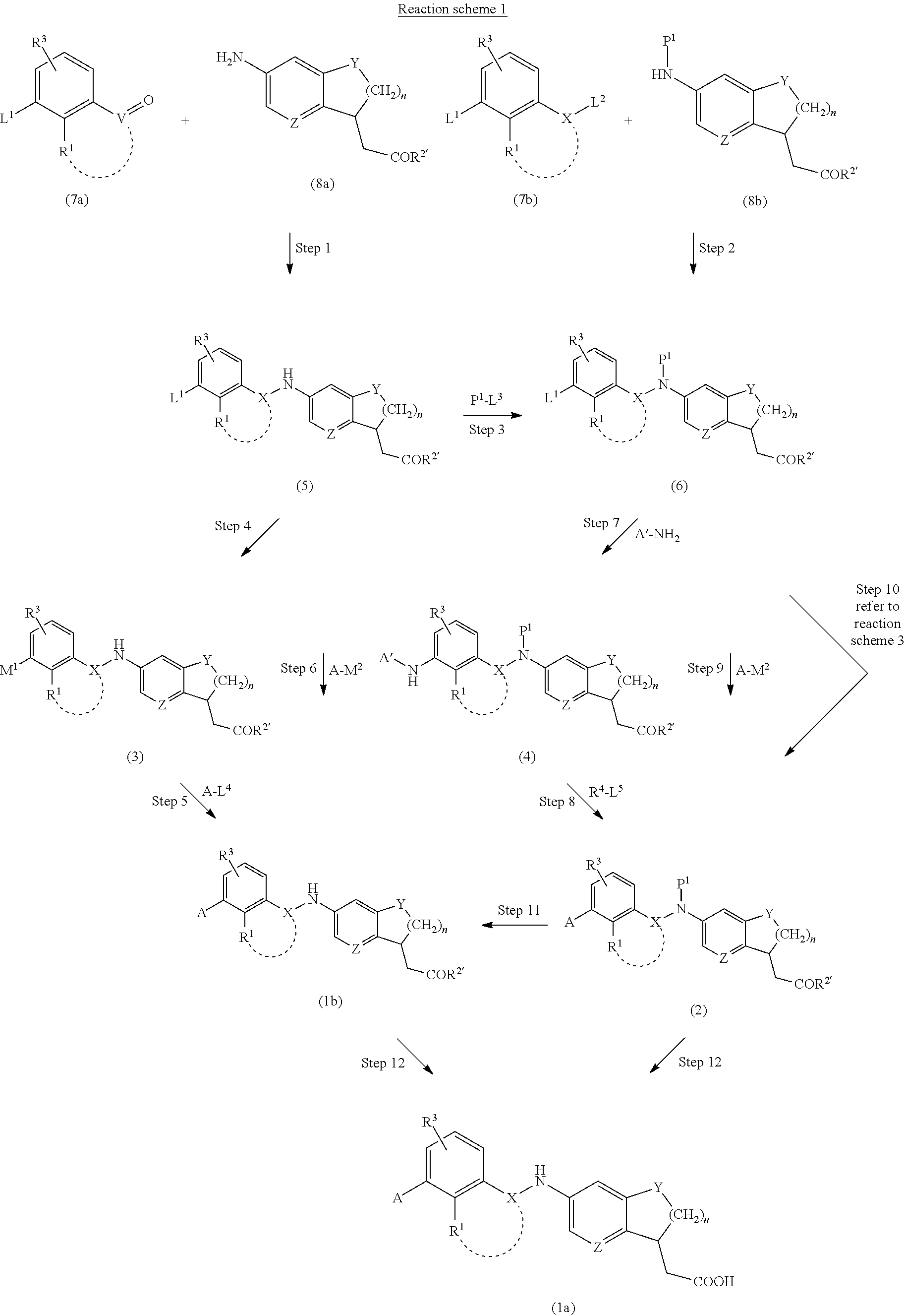

### Step 1

Compound (5) can be produced by reacting compound (7a) with compound (8a).

In step 1, L¹ is a leaving group, V is CH, or a carbon atom forming a ring together with R¹, R^{2′} is a substituent, and other symbols are as defined above.

Examples of the leaving group for L¹ include a halogen atom (e.g., fluorine, chlorine, bromine, iodine), optionally halogenated C₁₋₆ alkylsulfonyloxy (e.g., methanesulfonyloxy, ethanesulfonyloxy, trichloromethanesulfonyloxy, trifluoromethanesulfonyloxy), arylsulfonyloxy optionally having substituent(s) (e.g., C₆₋₁₀ arylsulfonyloxy (e.g., phenylsulfonyloxy, naphthylsulfonyloxy) optionally substituted by 1 to 3 substituents selected from C₁₋₆ alkyl group (e.g., methyl, ethyl), C₁₋₆ alkoxy (e.g., methoxy, ethoxy) and a nitro group, and the like, specific examples include phenylsulfonyloxy, m-nitrophenylsulfonyloxy, p-toluenesulfonyloxy and the like, acyloxy (e.g., trichloroacetoxy, trifluoroacetoxy and the like) and the like.

Compound (5) can be produced by subjecting compound (7a) and compound (8a) to a reductive amination reaction (e.g., the methods described in Jikken Kagaku Kouza, the 4th Edition, vol. 20, pages 282-284 and 366-368 (The Chemical Society of Japan ed.); J. Am. Chem. Soc., vol. 93, pages 2897-2904, 1971; Synthesis, page 135, 1975, and the like).

In the reductive amination reaction, compound (7a) and compound (8a) are subjected to a dehydration reaction to give an imine form, and the imine form is subjected to a reduction reaction to give compound (5).

The dehydrating reaction is promoted by adding a dehydrating agent such as molecular sieves and the like, or p-toluenesulfonic acid, zinc chloride, phosphoryl chloride, boron trifluoride, titanium tetrachloride, acetic acid, trifluoroacetic acid and the like to the system, removing water from the system by using Dean-Stark apparatus and the like, or a combination of these.

The reduction reaction is generally carried out by using a reducing agent according to a conventional method. Examples of the reducing agent include metal hydrides such as aluminum hydride, diisobutylaluminum hydride, tributyltin hydride and the like; metal hydride complex compounds such as sodium cyanoborohydride, sodium triacetoxyborohydride, sodium borohydride, lithium aluminum hydride and the like; borane complexes such as borane tetrahydrofuran complex, borane dimethylsulfide complex, picoline-borane complex and the like; alkylboranes such as hexylborane, disiamylborane and the like; diborane; metals such as zinc, aluminum, tin, iron and the like; alkali metal such as sodium, lithium etc./liquid ammonia (Birch reduction) and the like.

The amount of the reducing agent to be used is appropriately determined depending on the kind of the reducing agent. For example, the amount of the metal hydride, metal hydride complex compound, borane complex, alkylboranes or diborane to be used is each generally about 0.25-about 10 mol, preferably about 0.5-about 5 mol, per 1 mol of compound (7a), and the amount of the metals (containing alkali metal to be used in Birch reduction) is generally about 1-about 20 mol, preferably about 1-about 5 mol, per 1 mol of compound (7a).

The reduction reaction can also be carried out by a hydrogenation reaction. In this case, for example, catalysts such as palladium carbon, palladium black, platinum dioxide, Raney-nickel, Raney-cobalt and the like can be used. The amount of the catalyst to be used is generally about 5 to about 1000 wt %, preferably about 10 to about 300 wt %, relative to compound (7a).

The hydrogenation reaction can also be carried out using various hydrogen sources instead of gaseous hydrogen. As the hydrogen source, for example, formic acid, ammonium formate, triethylammonium formate, sodium phosphinate, hydrazine and the like can be mentioned. The amount of the hydrogen source to be used is generally about 1 to about 10 mol, preferably about 1 to about 5 mol, per 1 mol of compound (7a).

The reaction is advantageously carried out using a solvent inert to the reaction. While the solvent is not particularly limited as long as the reaction proceeds, for example, halogenated hydrocarbons (e.g., dichloromethane, chloroform, carbon tetrachloride, 1,2-dichloroethane and the like); alcohols such as methanol, ethanol, 1-propanol, 2-propyl alcohol, tert-butyl alcohol and the like; ethers such as diethyl ether, diisopropyl ether, diphenyl ether, tetrahydrofuran, 1,4-dioxane, 1,2-dimethoxyethane and the like; esters such as ethyl acetate, tert-butyl acetate and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoryl amide and the like; organic acids such as formic acid, acetic acid, propanoic acid, trifluoroacetic acid, methanesulfonic acid and the like, a mixed solvent thereof and the like are preferable.

The amount of compound (8a) to be used is generally about 0.2-about 5 mol, preferably about 0.5-about 2 mol, per 1 mol of compound (7a).

While the reaction time varies depending on the reagent and solvent to be used, it is generally about 10 min to about 100 hr, preferably about 30 min to about 50 hr. The reaction temperature is generally about −20° C. to about 100° C., preferably about 0° C. to about 80° C.

### Step 2

Compound (6) can be produced by reacting compound (7b) with compound (8b).

In step 2, L² is hydroxy or a leaving group, P¹ is acyl, and other symbols are as defined above.

Examples of the leaving group for L² include those recited for the leaving group L.

Examples of the acyl for P¹ include carbonyl such as trifluoroacetyl, trichloroacetyl and the like; sulfonyl such as 2-nitrobenzenesulfonyl, 4-nitrobenzenesulfonyl, 2,4-dinitrobenzenesulfonyl, methanesulfonyl, ethanesulfonyl, benzenesulfonyl, p-toluenesulfonyl etc. and the like.

(i) When L² is hydroxy, compound (6) can be produced by subjecting compound (7b) and compound (8b) to Mitsunobu reaction (for example, the methods described in Synthesis, page 1-27, 1981, Tetrahedron Lett., vol. 36, page 6373-6374, 1995, Tetrahedron Lett., vol. 38, page 5831-5834, 1997 and the like). In this reaction, compound (7b) is reacted with compound (8b) in the presence of azodicarboxylate such as diethyl azodicarboxylate, diisopropyl azodicarboxylate, 1,1′-(azodicarbonyl)dipiperidine and the like and phosphine such as triphenylphosphine, tributylphosphine and the like.

The reaction is advantageously carried out using a solvent inert to the reaction. While such solvent is not particularly limited as long as the reaction proceeds and, for example, ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoryl triamide and the like; halogenated hydrocarbons (e.g., dichloromethane, chloroform, carbon tetrachloride, 1,2-dichloroethane and the like); nitriles such as acetonitrile, propionitrile and the like; ketones such as acetone, methyl ethyl ketone and the like; esters such as ethyl acetate, tert-butyl acetate and the like; sulfoxides such as dimethyl sulfoxide and the like and the like, or a mixed solvent thereof and the like are preferable.

The reaction time is generally 5 min to 100 hr, preferably 30 min to 72 hr. The reaction temperature is generally −20° C. to 200° C., preferably 0° C. to 100° C.

The amount of compound (8b) to be used is about 0.5-5 mol, preferably about 0.9-2 mol, per 1 mol of compound (7b).

The amount of each of the azodicarboxylates and phosphines to be used is about 1-5 mol, preferably about 1-2 mol, per 1 mol of compound (8b).

(ii) When L² is a leaving group, compound (6) can be produced by reacting compound (7b) with compound (8b) in the presence of a base.

Examples of the base include alkali metal hydroxides such as lithium hydroxide, sodium hydroxide, potassium hydroxide and the like; alkaline earth metal hydroxides such as barium hydroxide and the like; alkali metal carbonates such as sodium carbonate, potassium carbonate, cesium carbonate and the like; alkali metal hydrogen carbonates such as sodium hydrogen carbonate and the like; acetates such as sodium acetate, ammonium acetate and the like; aromatic amines such as pyridine, 2,6-lutidine and the like; tertiary amines such as triethylamine, tripropylamine, tributylamine, N,N-diisopropylethylamine, cyclohexyldimethylamine, 4-dimethylaminopyridine, N,N-dimethylaniline, N-methylpiperidine, N-methylpyrrolidine, N-methylmorpholine and the like; alkali metal hydrides such as sodium hydride, potassium hydride and the like; metal amides such as sodium amide, lithium diisopropylamide, lithium hexamethyl disilazide and the like; alkali metal alkoxides having 1 to 6 carbon atoms such as sodium methoxide, sodium ethoxide, sodium tert-butoxide, potassium tert-butoxide etc. and the like.

The reaction is advantageously carried out using a solvent inert to the reaction. While such solvent is not particularly limited as long as the reaction proceeds and, for example, ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoryl triamide and the like; halogenated hydrocarbons (e.g., dichloromethane, chloroform, carbon tetrachloride, 1,2-dichloroethane and the like); nitriles such as acetonitrile, propionitrile and the like; ketones such as acetone, methyl ethyl ketone and the like; esters such as ethyl acetate, tert-butyl acetate and the like; sulfoxides such as dimethyl sulfoxide and the like; aromatic amines such as pyridine and the like; tertiary amines such as triethylamine, N,N-diisopropylethylamine and the like; and the like or a mixed solvent thereof and the like are preferable.

The amount of compound (8b) to be used is about 0.8-10 mol, preferably about 0.9-2 mol, per 1 mol of compound (7b). In addition, the amount of the base to be used is about 1-10 mol, preferably about 1-3 mol, per 1 mol of compound (8b).

The reaction time is generally 10 min to 12 hr, preferably 20 min to 6 hr. The reaction temperature is generally −70° C. to 250° C., preferably −20° C. to 100° C.

### Step 3

Compound (6) can also be produced by reacting compound (5) with compound P¹-L³.

In step 3, L³ is a leaving group, and other symbols are as defined above.

Examples of the leaving group L³ include those recited for leaving group L¹.

Compound (6) can be produced according to a method known per se, for example, by reacting compound (5) with compound P¹-L³ in the presence of a base.

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; organic bases such as trimethylamine, triethylamine, N,N-diisopropylethylamine, pyridine, picoline, 2,6-lutidine, 4-dimethylaminopyridine, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene etc. and the like.

The reaction is advantageously carried out using a solvent inert to the reaction. While such solvent is not particularly limited as long as the reaction proceeds, for example, ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoryl triamide and the like; halogenated hydrocarbons (e.g., dichloromethane, chloroform, carbon tetrachloride, 1,2-dichloroethane and the like); nitriles such as acetonitrile, propionitrile and the like; ketones such as acetone, methyl ethyl ketone and the like; sulfoxides such as dimethyl sulfoxide and the like; and the like or a mixed solvent thereof and the like are preferable.

While the reaction time varies depending on the reagent and solvent to be used, it is generally 10 min to 100 hr, preferably 30 min to 50 hr.

The reaction temperature is generally −30° C. to 100° C., preferably 0° C. to 80° C.

The amount of compound P¹-L³ to be used is about 0.5-5 mol, preferably about 1-3 mol, per 1 mol of compound (5).

The amount of the base to be used is about 0.5-10 mol, preferably about 1-5 mol, per 1 mol of compound (5).

### Step 4

Compound (3) can be produced by reacting compound (5) with a metal or an organic metal reagent.

In step 4, M¹ is a metal (e.g., boron, tin, silicon, potassium, sodium, lithium, aluminum, magnesium, copper, mercury, zinc, thallium and the like, which may be formed as a complex), and other symbols are as defined above.

Compound (3) can be produced, for example, according to the methods described in Palladium Reagents and Catalysts, John Wiley and Sons, page 289-293 (preparation of organic boron compound), page 313-317 (preparation of organotin compound), page 338-340 (preparation of organic silicon compound), 2004 or a method analogous thereto.

When M¹ is, for example, boron, compound (5) is reacted with organic boron reagents such as bis(pinacolato)diboron, pinacolborane and the like in the presence of a transition metal catalyst and base.

Examples of the transition metal catalyst include palladium (II) acetate, tris(dibenzylideneacetone)dipalladium, tetrakis(triphenylphosphine)palladium (0), bis(triphenylphosphine)palladium (II) dichloride, [1,1′-bis(diphenylphosphino)ferrocene]palladium (II) dichloride and the like. The amount of the transition metal catalyst to be used is about 0.000001-5 mol, preferably about 0.0001-1 mol, per 1 mol of compound (5). When a metal catalyst unstable to oxygen is used for the reaction, the reaction is preferably carried out under an inert gas (e.g., argon gas or nitrogen gas) atmosphere or stream. The reaction may be advantageously carried out in the co-presence of about 1-50 mol, preferably about 1-20 mol, of a phosphine ligand relative to a transition metal catalyst. Examples of the phosphine ligand include triphenylphosphine, 1,1′-bis(diphenylphosphino)ferrocene, 2,2′-bis(diphenylphosphino)-1,1′-binaphthyl, bis(2-diphenylphosphinophenyl)ether and the like.

Examples of the base include alkali metal acetates such as lithium acetate, sodium acetate, potassium acetate and the like; alkali metal phenoxides such as lithium phenoxide, sodium phenoxide, potassium phenoxide and the like; alkali metal alkoxides having 1 to 6 carbon atoms such as sodium methoxide, sodium ethoxide, sodium tert-butoxide and the like; alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal phosphate such as sodium phosphate, potassium phosphate and the like; organic bases such as trimethylamine, triethylamine, N,N-diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene etc. and the like.

The reaction proceeds advantageously in a solvent inert to the reaction. Preferable examples of the solvent include ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoric triamide and the like; halogenated hydrocarbons (e.g., dichloromethane, chloroform, carbon tetrachloride, 1,2-dichloroethane and the like); nitriles such as acetonitrile, propionitrile and the like; ketones such as acetone, methyl ethyl ketone and the like; sulfoxides such as dimethyl sulfoxide and the like; water and the like or a mixed solvent thereof and the like.

The amount of the organic boron reagent to be used is about 1-20 mol, preferably about 1-5 mol, per 1 mol of compound (5). In addition, the amount of the base to be used is about 1-20 mol, preferably about 1-5 mol, per 1 mol of compound (5).

The reaction temperature is −10° C. to 250° C., preferably 0° C. to 200° C.

While the reaction time varies depending on the kind of compound (5), organic metal reagent, transition metal catalyst, ligand, base or solvent, the reaction temperature and the like, it is generally 1 min-200 hr, preferably 5 min-100 hr.

### Step 5

Compound (1b) can be produced by reacting compound (3) with compound A-L⁴.

In step 5, L⁴ is a leaving group, and other symbols are as defined above.

Examples of the leaving group for L⁴ include those recited for leaving group L¹.

Compound (3) and compound A-L⁴ are generally reacted in the presence of a base. Examples of the base include alkali metal hydrides such as sodium hydride, potassium hydride and the like; alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; alkali metal phosphates such as sodium phosphate, potassium phosphate and the like; alkali metal alkoxides having 1 to 6 carbon atoms such as sodium methoxide, sodium ethoxide, sodium tert-butoxide and the like; organic bases such as trimethylamine, triethylamine, N,N-diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like; organic lithiums such as methyllithium, n-butyllithium, sec-butyllithium, tert-butyllithium and the like; lithium amides such as lithium diisopropylamide etc. and the like.

The reaction is advantageously carried out using a solvent inert to the reaction. While such solvent is not particularly limited as long as the reaction proceeds, for example, alcohols such as methanol, ethanol, propanol, isopropanol, butanol, tert-butanol and the like; ethers such as dioxane, tetrahydrofuran, diethyl ether, tert-butyl methyl ether, diisopropyl ether, ethylene glycol-dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoryl amide; water and the like and a mixed solvent thereof and the like are preferable.

The reaction can be generally promoted by using a transition metal catalyst. As the transition metal catalyst, a metal complex having various ligands is used and, for example, palladium compounds [e.g., palladium (II) acetate, tris(dibenzylideneacetone)dipalladium, tetrakis(triphenylphosphine)palladium (0), bis(triphenylphosphine)palladium (II) dichloride, [1,1′-bis(diphenylphosphino)ferrocene]palladium (II) dichloride, bis(triethylphosphine)palladium (II) dichloride and the like], nickel compounds [e.g., tetrakis(triphenylphosphine) nickel (0), bis(triethylphosphine)nickel (II) dichloride, bis(triphenylphosphine)nickel (II) dichloride and the like], rhodium compounds [e.g., tris(triphenylphosphine)rhodium (III) trichloride and the like], cobalt compound, copper compounds [e.g., copper oxide, copper (II) chloride and the like], platinum compound and the like can be mentioned. Among these, palladium compound, nickel compound and copper compound are preferable. The amount of the transition metal catalyst to be used is about 0.000001-5 mol, preferably about 0.0001-1 mol, per 1 mol of compound (A-L⁴). When a metal catalyst unstable to oxygen is used for the reaction, the reaction is preferably carried out under an inert gas (e.g., argon gas or nitrogen gas) atmosphere or stream. The reaction may be advantageously carried out in the co-presence of a ligand such as phosphine and the like relative to a transition metal catalyst. For example, triphenylphosphine, 1,1′-bis(diphenylphosphino)ferrocene, 2,2′-bis(diphenylphosphino)-1,1′-binaphthyl, 2-dicyclohexylphosphino-2′,6′-dimethoxybiphenyl, 2-dicyclohexylphosphino-2′,4′,6′-triisopropylbiphenyl, bis(2-diphenylphosphinophenyl)ether, 9,9-dimethyl-4,5-bis(diphenylphosphino)xanthene and the like can be mentioned. The amount of the ligand to be used is generally 1 to 50 equivalents, preferably 1 to 10 equivalents, per 1 equivalent of a transition metal catalyst.

The amount of compound A-L⁴ to be used is about 0.1-10 mol, preferably about 0.5-2 mol, per 1 mol of compound (3). In addition, the amount of the base to be used is about 1-20 mol, preferably about 1-5 mol, per 1 mol of compound (3).

The reaction temperature is −10° C.-250° C., preferably 0° C.-150° C.

While the reaction time varies depending on the kind of compound (3), compound A-L⁴, metal catalyst, base or solvent, the reaction temperature and the like, it is generally 1 min-200 hr, preferably 5 min-100 hr.

### Step 6

Compound (1b) can be produced by reacting compound (5) with compound A-M² according to the method exemplified in step 5 or a method analogous thereto.

In step 6, M² is a metal (e.g., boron, tin, silicon, potassium, sodium, lithium, aluminum, magnesium, copper, mercury, zinc, thallium and the like, which may be formed as a complex), and other symbols are as defined above.

### Step 7

Compound (4) can be produced by reacting compound (6) with compound A′-NH₂.

In step 7, A′ is an aromatic ring group optionally having substituent(s) or C₁₋₆ alkyl optionally having substituent(s), and other symbols are as defined above.

Examples of the aromatic ring group optionally having substituent(s) or C₁₋₆ alkyl optionally having substituent(s) for A′ include 4-methylpyridin-2-yl, 2-pyrimidinyl, benzyl and the like.

Compound (6) and compound A′-NH₂ are generally reacted in the presence of a base. Examples of the base include alkali metal hydrides such as sodium hydride, potassium hydride and the like; alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; alkali metal phosphates such as sodium phosphate, potassium phosphate and the like; alkali metal alkoxides having 1 to 6 carbon atoms such as sodium methoxide, sodium ethoxide, sodium tert-butoxide and the like; organic bases such as trimethylamine, triethylamine, N,N-diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like; organic lithiums such as methyllithium, n-butyllithium, sec-butyllithium, tert-butyllithium and the like; lithium amides such as lithium diisopropylamide etc. and the like.

The reaction is advantageously carried out using a solvent inert to the reaction. While such solvent is not particularly limited as long as the reaction proceeds, for example, alcohols such as methanol, ethanol, propanol, isopropanol, butanol, tert-butanol and the like; ethers such as dioxane, tetrahydrofuran, diethyl ether, tert-butyl methyl ether, diisopropyl ether, ethylene glycol-dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoryl amide; water and the like and a mixed solvent thereof and the like are preferable.

The reaction can be generally promoted by using a transition metal catalyst. As the transition metal catalyst, a metal complex having various ligands, for example, palladium compounds [e.g., palladium (II) acetate, tris(dibenzylideneacetone)dipalladium, tetrakis(triphenylphosphine)palladium (0), bis(triphenylphosphine)palladium (II) dichloride, [1,1′-bis(diphenylphosphino)ferrocene]palladium (II) dichloride, bis(triethylphosphine)palladium (II) dichloride and the like], nickel compounds [e.g., tetrakis(triphenylphosphine)nickel (0), bis(triethylphosphine)nickel (II) dichloride, bis(triphenylphosphine)nickel (II) dichloride and the like], rhodium compounds [e.g., tris(triphenylphosphine)rhodium (III) trichloride and the like], cobalt compound, copper compounds [e.g., copper oxide, copper (II) chloride and the like], platinum compounds and the like can be mentioned. Among these, palladium compound, nickel compound and copper compound are preferable. The amount of the transition metal catalyst to be used is about 0.000001-5 mol, preferably about 0.0001-1 mol, per 1 mol of compound (A-L⁴). When a metal catalyst unstable to oxygen is used for the reaction, a reaction is preferably carried out under an inert gas (e.g., argon gas or nitrogen gas) atmosphere or stream. The reaction may be advantageously carried out in the co-presence of a ligand such as phosphine and the like relative to the above-mentioned transition metal catalyst. For example, triphenylphosphine, 1,1′-bis(diphenylphosphino)ferrocene, 2,2′-bis(diphenylphosphino)-1,1′-binaphthyl, 2-dicyclohexylphosphino-2′,6′-dimethoxybiphenyl, 2-dicyclohexylphosphino-2′,4′,6′-triisopropylbiphenyl, bis(2-diphenylphosphinophenyl)ether, 9,9-dimethyl-4,5-bis(diphenylphosphino)xanthene and the like can be mentioned. The amount of the ligand to be used is generally 1 to 50 equivalents, preferably 1 to 10 equivalents, per 1 equivalent of a transition metal catalyst.

The amount of the compound A′-NH₂ to be used is about 0.5-10 mol, preferably about 0.5-5 mol, per 1 mol of compound (6). In addition, the amount of the base to be used is about 1-20 mol, preferably about 1-5 mol, per 1 mol of compound A′-NH₂.

The reaction temperature is −10° C.-250° C., preferably 0° C.-150° C.

While the reaction time varies depending on the kind of compound (6), compound A′ —NH₂, the metal catalyst, base or solvent, the reaction temperature and the like, it is generally 1 min-200 hr, preferably 5 min-100 hr.

### Step 8

Compound (2) can be produced by reacting compound (4) with compound R⁴-L⁵.

In step 8, R⁴ is C₁₋₆ alkyl optionally having substituent(s), L⁵ is a leaving group, and other symbols are as defined above.

Examples of the C₁₋₆ alkyl optionally having substituent(s) for R⁴ include ethyl, n-propyl, cyclopropyl, methoxyethyl and the like.

Examples of the leaving group for L⁵ include those recited for leaving group L.

Compound (2) can be produced according to a method known per se, for example, by reacting compound (4) with compound R⁴-L⁵ in the presence of a base.

Examples of the base include alkali metal hydrides such as sodium hydride, potassium hydride and the like; alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; organic bases such as trimethylamine, triethylamine, N,N-diisopropylethylamine, pyridine, picoline, 2,6-lutidine, 4-dimethylaminopyridine, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene etc. and the like.

The reaction is advantageously carried out by using a solvent inert to the reaction. While such solvent is not particularly limited as long as the reaction proceeds, for example, ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoric triamide and the like; halogenated hydrocarbons (e.g., dichloromethane, chloroform, carbon tetrachloride, 1,2-dichloroethane and the like); nitriles such as acetonitrile, propionitrile and the like; ketones such as acetone, methyl ethyl ketone and the like; sulfoxides such as dimethyl sulfoxide and the like and the like or a mixed solvent thereof and the like are preferable.

While the reaction time varies depending on the reagent and solvent to be used, generally 10 min-100 hr, preferably min-50 hr.

The reaction temperature is generally −30° C.-150° C., preferably 0° C.-100° C.

The amount of compound R⁴-L⁵ to be used is about 0.5-20 mol, preferably about 1-10 mol, per 1 mol of compound (4).

### Step 9

Compound (2) can be produced by reacting compound (6) with compound A-M² according to the method exemplified in step 5 or a method analogous thereto.

In step 9, each symbol is as defined above.

### Step 10

Compound (2) can be produced by reaction according to the method of reaction scheme 3 to be mentioned below.

### Step 11

Compound (1b) can also be produced by eliminating P¹ of compound (2).

The protecting group P¹ can be removed from compound (2) according to a method known per se, for example, the methods described in Protective Groups in Organic Synthesis, John Wiley and Sons (1980), Tetrahedron Lett., vol. 36, page 6373-6374, 1995, Tetrahedron Lett., vol. 38, page 5831-5834, 1997, Journal of Synthetic Organic Chemistry, Japan, vol. 59, page 779-789, 2001 and the like or a method analogous thereto. For removal of the protecting group P¹, a method using acid, base etc. and the like can be employed.

### Step 12

Compound (1a) can be produced by subjecting compound (1b) or compound (2) to hydrolysis.

In step 12, each symbol is as defined above.

The hydrolysis is performed according to a conventional method and using an acid or base.

Examples of the acid include mineral acids such as hydrochloric acid, sulfuric acid and the like; Lewis acids such as boron trichloride, boron tribromide and the like; organic acids such as trifluoroacetic acid, p-toluenesulfonic acid etc. and the like. Here, Lewis acid can also be used in combination with thiol or sulfide.

Examples of the base include alkali metal hydroxides such as lithium hydroxide, sodium hydroxide, potassium hydroxide, barium hydroxide and the like; alkali metal carbonates such as sodium carbonate, potassium carbonate and the like; alkali metal alkoxides having 1 to 6 carbon atoms such as sodium methoxide, sodium ethoxide, potassium tert-butoxide etc. and the like. The amount of the acid or base to be used is about 0.5-10 mol, preferably about 0.5-6 mol, per 1 mol of compound (1b) or compound (2).

The hydrolysis is carried out without a solvent or using a solvent inert to the reaction. While such solvent is not particularly limited as long as the reaction proceeds, for example, ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; alcohols such as methanol, ethanol, 2-propyl alcohol and the like; halogenated hydrocarbons (e.g., dichloromethane, chloroform, carbon tetrachloride, 1,2-dichloroethane and the like); water and the like or a mixed solvent thereof and the like are preferable.

The reaction time is generally 10 min-100 hr, preferably 10 min-24 hr. The reaction temperature is generally −10° C.-200° C., preferably 0° C.-120° C.

Compound (7a), (7b), P¹-L³, A-L⁴, A-M², A′-NH₂ and R⁴-L⁵ to be used in the reaction scheme 1 can be obtained as commercially available products, or can be produced according to a method known per se or a method analogous thereto.

For example, of compounds (5), compound (5-1) wherein the ring formed by R¹ and the substituent of X is a 5-membered ring can be produced, for example, according to the method shown in reaction scheme 2 or a method analogous thereto.

In reaction scheme 2, each symbol is as defined above.

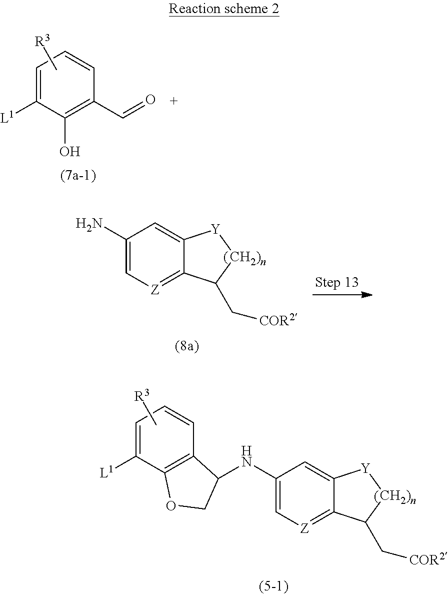

### Step 13

Compound (5-1) can be produced by subjecting an imine compound produced by a dehydrating reaction with compound (7a-1) and compound (8a) to Corey-Chaykovsky reaction.

The imine compound can be synthesized from compound (7a-1) and compound (8a) in the same manner as in the dehydration reaction of compound (7a) and compound (8a) exemplified in step 1.

The Corey-Chaykovsky reaction is generally carried out according to a conventional method and using dimethylsulfoxonium methylide prepared from halogenated trimethylsulfoxonium and a base. Examples of the halogenated trimethylsulfoxonium include trimethylsulfoxonium iodide, trimethylsulfoxonium bromide, trimethylsulfoxonium chloride and the like. In addition, halogenated trimethylsulfonium can be used instead of halogenated trimethylsulfoxonium. The amount of the halogenated trimethylsulfoxonium or halogenated trimethylsulfonium to be used is generally about 1-10 mol, preferably about 1-5 mol, per 1 mol of compound (7a-1).

Examples of the base include alkali metal hydrides such as sodium hydride, potassium hydride and the like; alkali metal alkoxides having 1 to 6 carbon atoms such as sodium methoxide, sodium ethoxide, sodium tert-butoxide, potassium tert-butoxide and the like; metal amides such as sodium amide, lithium diisopropylamide, lithium hexamethyl disilazide etc. and the like. The amount of the base to be used is generally about 1-10 mol, preferably about 1-5 mol, per 1 mol of compound (7a-1).

The reaction is advantageously carried out by using a solvent inert to the reaction. While such solvent is not particularly limited as long as the reaction proceeds, for example, ethers such as diethyl ether, diisopropyl ether, diphenyl ether, tetrahydrofuran, 1,4-dioxane, 1,2-dimethoxyethane and the like; sulfoxides such as dimethyl sulfoxide and the like and the like or a mixed solvent thereof and the like are preferable.

The reaction time is generally 5 min-100 hr, preferably 10 min-72 hr. The reaction temperature is generally −20° C.-200° C., preferably −10° C.-100° C.

For example, of compounds (2) shown in reaction scheme 1, compound (2-1) or compound (2-2) wherein A is a benzoimidazole ring can be produced, for example, according to the method shown in reaction scheme 3 or a method analogous thereto.

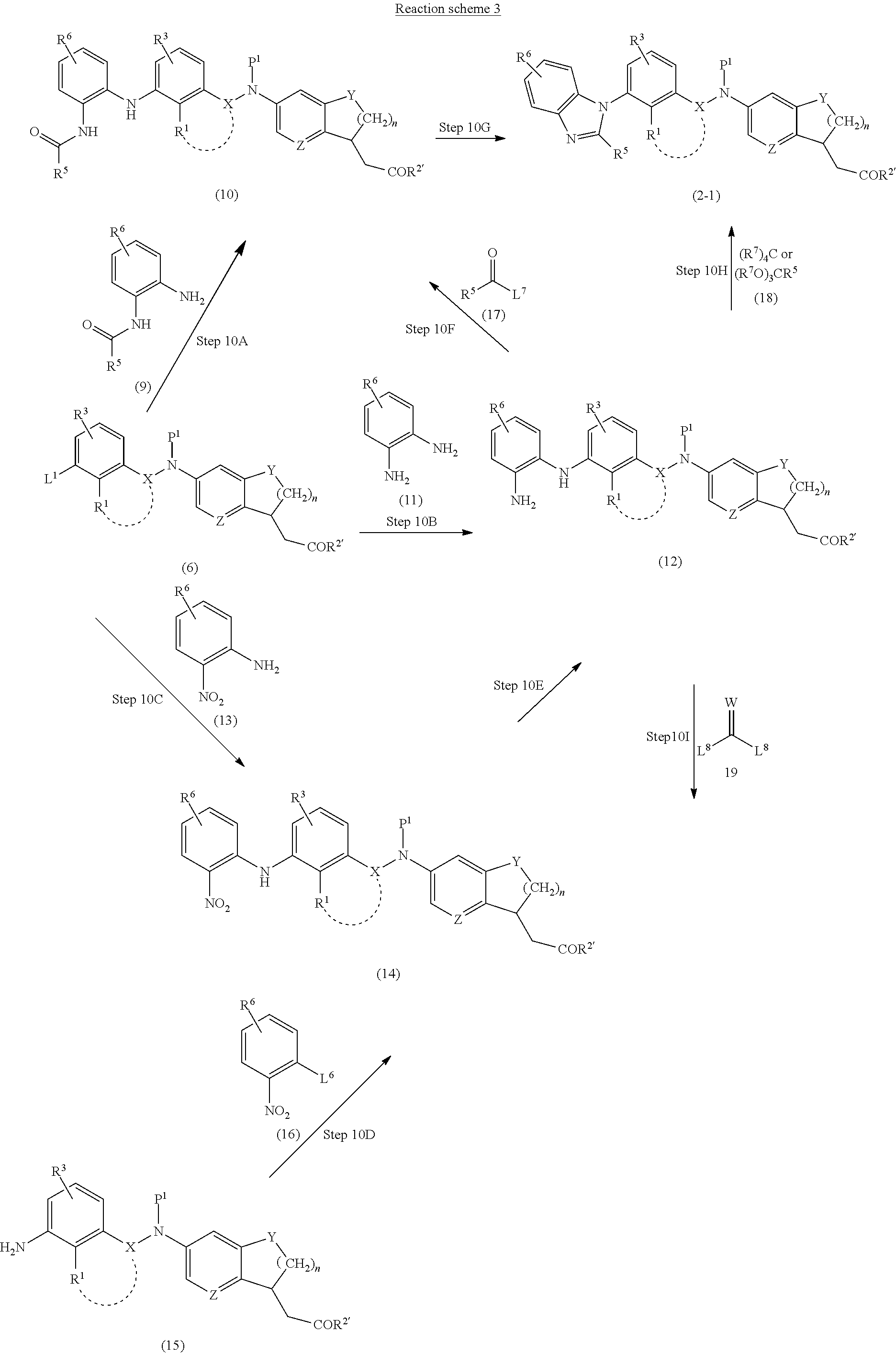

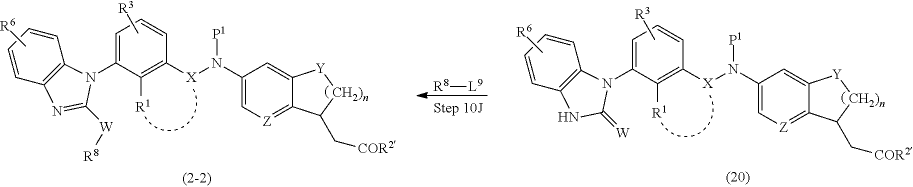

### Step A10A

Compound (10) can be produced by reacting compound (6) with compound (9) according to the method exemplified in step 7 or

In step 10A, R⁵ is hydrogen, C₁-C₆ alkyl (e.g., methyl, ethyl, n-propyl) optionally having substituent(s), C₁-C₆ alkoxy (e.g., methoxy, ethoxy, n-propoxy) optionally having substituent(s), C₃-C₁₀ cycloalkyl (e.g., cyclopropyl, cyclobutyl, cyclopentyl) optionally having substituent(s), aryl (e.g., phenyl, naphthyl) optionally having substituent(s), or a 4- to 7-membered heterocyclic group optionally having substituent(s) (e.g., 2-tetrahydrofuranyl, 4-tetrahydro-2H-pyranyl, 5-methylfuran-2-yl), R⁶ shows 0 to 4 substituents selected from a halogen atom (e.g., fluorine, chlorine, bromine, iodine), a C₁-C₆ alkyl group optionally having substituent(s) (e.g., methyl, ethyl, trifluoromethyl), C₃-C₁₀ cycloalkyl (e.g., cyclopropyl, cyclobutyl, cyclopentyl) optionally having substituent(s), C₁-C₆ alkoxy optionally having substituent(s) (e.g., methoxy, trifluoromethoxy, methylsulfonylpropoxy), C₁-C₆ alkylthio (e.g., methylthio, ethylthio) optionally having substituent(s), amino optionally having substituent(s) (e.g., dimethylamino, piperidinyl, morpholinyl), aryl (e.g., phenyl) optionally having substituent(s), a 4- to 7-membered heterocyclic group optionally having substituent(s) (e.g., 2-tetrahydrofuranyl, 4-tetrahydro-2H-pyranyl, 5-methylfuran-2-yl), and 4- to 7-membered heterocyclyl-oxy (e.g., tetrahydro-2H-pyran-4-yloxy) optionally having substituent(s), and other symbols are as defined above.

### Step 10B

Compound (12) can be produced by reacting compound (6) with compound (11) according to the method exemplified in step 7 or a method analogous thereto.

In step 10B, each symbol is as defined above.

### Step 10C

Compound (14) can be produced by reacting compound (6) with compound (13) according to the method exemplified in step 7 or a method analogous thereto.

In step 10C, each symbol is as defined above.

### Step 10D

Compound (14) can be produced by reacting compound (15) with compound (16) according to the method exemplified in step 7 or a method analogous thereto.

In step 10D, L⁶ is a leaving group, and other symbols are as defined above.

Examples of the leaving group for L⁶ include those recited for leaving group L¹.

### Step 10E

Compound (12) can be produced from compound (14).

In step 10E, each symbol is as defined above.

Compound (12) is converted to compound (14) according to a method known per se, for example, the method described in Reductions in Organic Chemistry, Second Edition, The American Chemical Society, page 95-97, 1996 or a method analogous thereto. For the substituent conversion, the hydrogenation reaction exemplified in step 1 and the like can be used.

### Step 10F

Compound (10) can also be produced by reacting compound (12) with compound (17).

In step 10F, L⁷ is hydroxy or a leaving group, and other symbols are as defined above.

Examples of the leaving group for L⁷ include halogen atoms (e.g., fluorine, chlorine, bromine, iodine), acyloxy (e.g., acetoxy, trifluoroacetoxy), aromatic heterocyclic groups (e.g., 1H-imidazol-1-yl) and the like.

(i) When L⁷ is hydroxy, compound (10) can be produced by directly condensing compound (12) and compound (17) using a condensing agent.

Examples of the condensing agent include carbodiimide type condensation reagents such as dicyclohexylcarbodiimide (DCC), diisopropylcarbodiimide (DIPC), 1-ethyl-3-(3-dimethylaminopropyl)carbodiimide (EDC), or hydrochloride thereof and the like; phosphoric acid type condensation reagents such as diethyl cyanophosphate, diphenylphosphoryl azide and the like; carbonyldiimidazole, 2-chloro-1,3-dimethylimidazolium tetrafluoroborate, 2-(7-aza-1H-benzotriazol-1-yl)-1,1,3,3-tetramethyluronium hexafluorophosphate (HATU) and the like.

Examples of the solvent to be used for the condensation reaction include amides such as N,N-dimethylformamide, N,N-dimethylacetamide, N-methylpyrrolidone and the like; sulfoxides such as dimethyl sulfoxide and the like; halogenated hydrocarbons such as chloroform, dichloromethane and the like; aromatic hydrocarbons such as benzene, toluene and the like; ethers such as tetrahydrofuran, dioxane, diethyl ether, dimethoxyethane and the like; esters such as methyl acetate, ethyl acetate and the like; nitriles such as acetonitrile, propionitrile and the like; water; and the like. These solvents may be used in a mixture at an appropriate ratio.

The amount of compound (17) to be used is generally 1-mol, preferably 1-3 mol, per 1 mol of compound (12).

The amount of the condensing agent to be used is generally 0.1-10 mol, preferably 0.3-3 mol, per 1 mol of compound (12).

When a carbodiimide type condensation reagent is used as the condensing agent, the reaction efficiency can be improved by using a suitable condensation promoter (e.g., 1-hydroxy-7-azabenzotriazole, 1-hydroxybenzotriazole, N-hydroxysuccinimide, N-hydroxyphthalimide) as necessary.

The reaction efficiency of the condensation reaction can be improved by using an organic amine base such as triethylamine, N,N-diisopropylethylamine, 4-(dimethylamino)-pyridine and the like.

The amount of each of such condensation promoter and organic amine base to be used is generally 0.1-10 mol, preferably 0.3-3 mol, per 1 mol of compound (12).

The reaction temperature is generally −30° C.-120° C., preferably −10° C.-100° C.

The reaction time is generally 0.5-60 hr.

(ii) When L⁷ is a leaving group, the reaction is generally performed in the presence of a base in a solvent that does not adversely influence the reaction.

Examples of the base include amines such as triethylamine, pyridine, N-methylmorpholine, N,N-dimethylaniline, 4-dimethylaminopyridine and the like; alkali metal salts such as lithium hydroxide, sodium hydroxide, potassium hydroxide, sodium hydrogen carbonate, sodium carbonate, potassium carbonate etc. and the like.

Examples of the solvent that does not adversely influence the reaction include amides such as N,N-dimethylformamide, N,N-dimethylacetamide, N-methylpyrrolidone and the like; sulfoxides such as dimethyl sulfoxide and the like; halogenated hydrocarbons such as chloroform, dichloromethane and the like; aromatic hydrocarbons such as benzene, toluene and the like; ethers such as tetrahydrofuran, dioxane, diethyl ether, dimethoxyethane and the like; esters such as methyl acetate, ethyl acetate and the like; nitriles such as acetonitrile, propionitrile and the like; water; and the like. These solvents may be used in a mixture at an appropriate ratio.

When the above-mentioned amides are used as a solvent, the reaction can also be performed in the absence of a base.

The amount of compound (17) to be used is generally 1-10 mol, preferably 1-5 mol, per 1 mol of compound (12).

The amount of the base to be used is generally 1-10 mol, preferably 1-5 mol, per 1 mol of compound (12).

The reaction temperature is generally −30° C.-120° C., preferably −10° C.-100° C.

The reaction time is generally 0.5-30 hr.

### Step 10G

Compound (2-1) can be produced from compound (10).

In step 10G, each symbol is as defined above.

Compound (2-1) can be produced according to a method known per se, for example, by reacting compound (10) in the presence of an acid.

Examples of the acid include organic acids such as acetic acid, p-toluenesulfonic acid and the like; mineral acids such as hydrochloric acid and the like; polyphosphoric acid and the like.

The reaction is carried our without a solvent or using a solvent inert to the reaction. While such solvent is not particularly limited as long as the reaction proceeds, for example, ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; halogenated hydrocarbons (e.g., dichloromethane, chloroform, carbon tetrachloride, 1,2-dichloroethane and the like) and the like or a mixed solvent thereof and the like are preferable.

The reaction time is generally 10 min-150 hr, preferably 10 min-48 hr. The reaction temperature is generally 0° C.-200° C., preferably 0° C.-150° C.

### Step 10H

Compound (2-1) can be produced by reacting compound (12) with compound (18).

In step 10H, R⁷ is C₁₋₆ alkyl optionally having substituent(s), and other symbols are as defined above.

Examples of the C₁₋₆ alkyl optionally having substituent(s) for R⁷ include methyl, ethyl, isopropyl and the like.

Compound (12) is generally reacted with compound (18) in a solvent inert to the reaction.

While the solvent is not particularly limited as long as the reaction proceeds, for example, alcohols such as methanol, ethanol, 1-propanol, 2-propyl alcohol and the like; ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; organic acids such as acetic acid, trifluoroacetic acid and the like and the like or a mixed solvent thereof and the like are preferable.

Where necessary, moreover, the reaction efficiency can be improved by using an acid such as acetic acid, p-toluenesulfonic acid and the like.

The reaction temperature is 0° C.-150° C., preferably 20° C.-80° C.

The reaction time is generally 5 min-72 hr, preferably 10 min-24 hr.

### Step 10I

Compound (20) can be produced by reacting compound (12) with compound (19).

In step 10I, W is an oxygen atom or a sulfur atom, L⁸ is a leaving group, and other symbols are as defined above.

Examples of the leaving group for L⁸ include halogen atoms (e.g., fluorine, chlorine, bromine, iodine), optionally halogenated C₁₋₆ alkyloxy (e.g., methoxy, ethoxy, trichloromethoxy), aromatic heterocyclic groups (e.g., 1H-imidazol-1-yl, 1H-1,2,4-triazol-1-yl) and the like.

### Step 10J

Compound (2-2) can be produced by reacting compound (20) with compound (R⁸-L⁹) according to the method exemplified in step 8 or a method analogous thereto.

In step 10J, R⁸ is C₁₋₆ alkyl optionally having substituent(s), L⁹ is a leaving group, and other symbols are as defined above.

Examples of the C₁₋₆ alkyl optionally having substituent(s) for R⁸ include methyl, ethyl, benzyl and the like.

Examples of the leaving group for L⁹ include those recited for leaving group L¹.

Compound (9), compound (11), compound (13), compound (16), compound (17), compound (18), compound (19) and compound R⁸-L⁹ to be used in the above-mentioned reaction scheme 3 can be easily obtained as commercially available products, or can be produced according to a method known per se.

For example, compound (8a) and compound (8b) shown in reaction scheme 1 can be produced, for example, according to the method shown in reaction scheme 4 or a method analogous thereto.

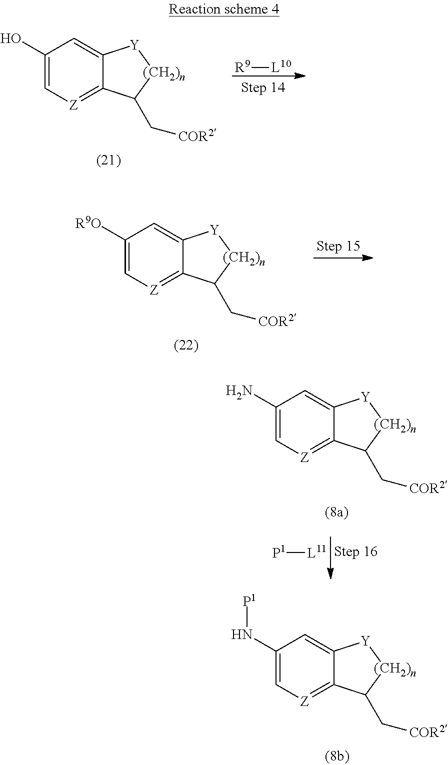

### Step 14

Compound (22) can be produced by reacting compound (21) with compound (R⁹-L¹⁰)

In step 14, R⁹ is C₁₋₆ alkylsulfonyl optionally having substituent(s), L¹⁰ is a leaving group, and other symbols are as defined above.

Examples of the C₁₋₆ alkylsulfonyl optionally having substituent(s) for R⁹ include methanesulfonyl, trifluoromethanesulfonyl, nonafluorobutanesulfonyl and the like.

Examples of the leaving group for L¹⁰ include those recited for leaving group L.

Compound (22) can be produced according to a method known per se, for example, by reacting compound (21) with compound R⁹-L¹⁰ in the presence of a base.

Examples of the base include organic bases such as trimethylamine, triethylamine, diisopropylethylamine, pyridine, picoline, 2,6-lutidine, 4-dimethylaminopyridine, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene etc. and the like.

The reaction is advantageously carried out without a solvent or using a solvent inert to the reaction. While such solvent is not particularly limited as long as the reaction proceeds, for example, ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; halogenated hydrocarbons (e.g., dichloromethane, chloroform, carbon tetrachloride, 1,2-dichloroethane and the like); aromatic amines such as pyridine and the like; tertiary amines such as triethylamine, N,N-diisopropylethylamine and the like and the like or a mixed solvent thereof and the like are preferable.

While the reaction time varies depending on the reagent and solvent to be used, it is generally 5 min-100 hr, preferably 30 min-50 hr.

The reaction temperature is generally −80° C.-100° C., preferably −30° C.-50° C.

The amount of compound R⁹-L¹⁰ to be used is about 1-10 mol, preferably about 1-3 mol, per 1 mol of compound (21).

### Step 15

Compound (8a) can be produced from compound (22).

Compound (8a) can be produced, for example, according to the method described in Palladium Reagents and Catalysts, John Wiley and Sons, pages 386-387, 2004 or a method analogous thereto.

### Step 16

Compound (8b) can be produced by reacting compound (8a) with compound (P¹-L¹¹) according to the method exemplified in step 3 or a method analogous thereto.

In step 16, L¹¹ is a leaving group, and other symbols are as defined above.

Examples of the leaving group for L¹¹ include those recited for leaving group L¹.

Compound (21), compound R⁹-L¹⁰ and compound P¹-L¹¹ to be used in the above-mentioned reaction scheme 4 can be easily obtained as commercially available products, or can be produced according to a method known per se. For example, methyl [(3S)-6-hydroxy-2,3-dihydro-1-benzofuran-3-yl]acetate can be produced according to WO2008/001931.

In the synthesis of compound (1b) or (2), the order of syntheses is not limited to that shown in reaction scheme 1 and, for example, the steps may be exchanged as appropriate as shown in reaction scheme 5.

In reaction scheme 5, each symbol is as defined above.

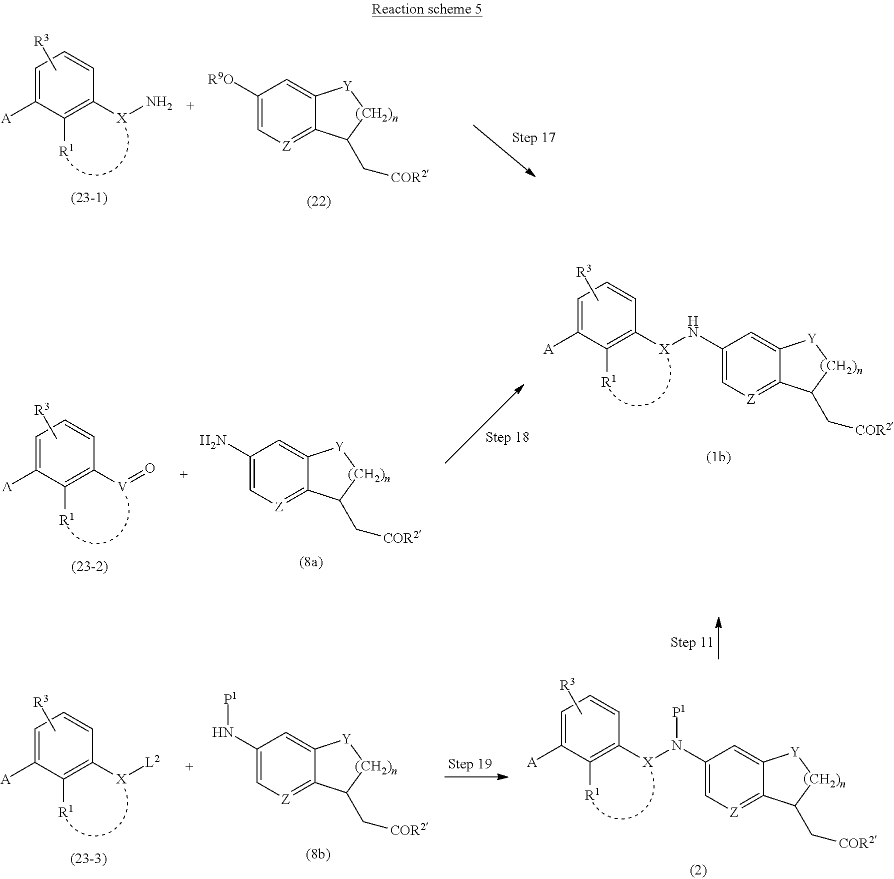

### Step 17

Compound (1b) can also be produced by reacting compound (23-1) with compound (22) according to the method exemplified in step 7 or a method analogous thereto.

### Step 18

Compound (1b) can also be produced by reacting compound (23-2) with compound (8a) according to the method exemplified in step 1 or a method analogous thereto.

### Step 19

Compound (2) can also be produced by reacting compound (23-3) with compound (8b) according to the method exemplified in step 2 or a method analogous thereto.

Compound (23-1), compound (23-2) and compound (23-3) to be used in the above-mentioned reaction scheme 5 can be produced by applying the reactions exemplified above, and further according to methods known per se such as introduction of substituent, functional group conversion reaction, protection and deprotection and the like.

In each of the aforementioned reactions, when the starting material compound has an amino group, a carboxyl group, a hydroxy group, a carbonyl group or a mercapto group as a substituent, a protecting group generally used in the peptide chemistry and the like may be introduced into these groups, and the object compound can be obtained by removing the protecting group as necessary after the reaction.

Examples of the amino-protecting group include a formyl group, a C₁₋₆ alkyl-carbonyl group, a C₁₋₆ alkoxy-carbonyl group, a benzoyl group, a C₇₋₁₀ aralkyl-carbonyl group (e.g., benzylcarbonyl), a C₇₋₁₄ aralkyloxy-carbonyl group (e.g., benzyloxycarbonyl, 9-fluorenylmethoxycarbonyl), a trityl group, a phthaloyl group, an N,N-dimethylaminomethylene group, a substituted silyl group (e.g., trimethylsilyl, triethylsilyl, dimethylphenylsilyl, tert-butyldimethylsilyl, tert-butyldiethylsilyl), a C₂₋₆alkenyl group (e.g., 1-allyl) and the like. These groups are optionally substituted by 1 to 3 substituents selected from a halogen atom, a C₁₋₆ alkoxy group and a nitro group.

Examples of the carboxyl-protecting group include a C₁₋₆ alkyl group, a C₇₋₁₁ aralkyl group (e.g., benzyl), a phenyl group, a trityl group, a substituted silyl group (e.g., trimethylsilyl, triethylsilyl, dimethylphenylsilyl, tert-butyldimethylsilyl, tert-butyldiethylsilyl), a C₂₋₆alkenyl group (e.g., 1-allyl) and the like. These groups are optionally substituted by 1 to 3 substituents selected from a halogen atom, a C₁₋₆ alkoxy group and a nitro group.

Examples of the hydroxyl-protecting group include a C₁₋₆ alkyl group, a phenyl group, a trityl group, a C₇₋₁₀ aralkyl group (e.g., benzyl), a formyl group, a C₁₋₆ alkyl-carbonyl group, a benzoyl group, a C₇₋₁₀ aralkyl-carbonyl group (e.g., benzylcarbonyl), a 2-tetrahydropyranyl group, a 2-tetrahydrofuranyl group, a substituted silyl group (e.g., trimethylsilyl, triethylsilyl, dimethylphenylsilyl, tert-butyldimethylsilyl, tert-butyldiethylsilyl), a C₂₋₆alkenyl group (e.g., 1-allyl) and the like. These groups are optionally substituted by 1 to 3 substituents selected from a halogen atom, a C₁₋₆ alkyl group, a C₁₋₆ alkoxy group and a nitro group.

Examples of the carbonyl-protecting group include cyclic acetal (e.g., 1,3-dioxane), non-cyclic acetal (e.g., di-C₁₋₆ alkylacetal) and the like.

Examples of the mercapto-protecting group include a C₁₋₆ alkyl group, a phenyl group, a trityl group, a C₇₋₁₀ aralkyl group (e.g., benzyl), a C₁₋₆ alkyl-carbonyl group, a benzoyl group, a C₇₋₁₀ aralkyl-carbonyl group (e.g., benzylcarbonyl), a C₁₋₆ alkoxy-carbonyl group, a C₆₋₁₄ aryloxy-carbonyl group (e.g., phenyloxycarbonyl), a C₇₋₁₄ aralkyloxy-carbonyl group (e.g., benzyloxycarbonyl, 9-fluorenylmethoxycarbonyl), a 2-tetrahydropyranyl group, a C₁₋₆ alkylamino-carbonyl group (e.g., methylaminocarbonyl, ethylaminocarbonyl) and the like. These groups are optionally substituted by 1 to 3 substituents selected from a halogen atom, a C₁₋₆ alkyl group, a C₁₋₆ alkoxy group and a nitro group.

The above-mentioned protecting group can be removed according to a method known per se, for example, the method described in Protective Groups in Organic Synthesis, John Wiley and Sons (1980). Specific examples include a method using acid, base, ultraviolet rays, hydrazine, phenylhydrazine, sodium N-methyldithiocarbamate, tetrabutylammonium fluoride, palladium acetate, trialkylsilyl halide (e.g., trimethylsilyl iodide, trimethylsilyl bromide) and the like, a reduction method and the like.

The present compound (I) obtained in each of the above-mentioned production methods can be isolated and purified by a known method such as concentration, concentration under reduced pressure, solvent extraction, crystallization, recrystallization, phase transfer, chromatography and the like. In addition, each starting material compound to be used in each of the above-mentioned production methods can be isolated and purified by a known method similar to the above. Alternatively, such starting material compound may also be used directly as a starting material without isolation for the next step.

When the compound (I) has isomers such as optical isomer, stereoisomer, positional isomer, rotational isomer and the like, and any isomers and mixture of isomers are encompassed in the compound (I). For example, when the compound (I) has an optical isomer, an optical isomer separated from a racemate is also encompassed in the compound (I). These isomers can be obtained as independent products by a synthesis means or a separation means (e.g., concentration, solvent extraction, column chromatography, recrystallization and the like), optical resolution means (e.g., fractional recrystallization method, chiral column method, diastereomer method and the like) and the like known per se.

The compound (I) may be a crystal, and both a single crystal and crystal mixtures are encompassed in the compound (I). Crystals can be produced by crystallization according to crystallization methods known per se.

The compound (I) may be a solvate (e.g., hydrate etc.) or a non-solvate (e.g., non-hydrate), both of which are encompassed in the compound (I).

A compound labeled with an isotope (e.g., ³H, ¹⁴C, ³⁵S, ¹²⁵I etc.) and the like is also encompassed in compound (I).

A deuterium-exchanged compound by converting ¹H to ²H(D) is also encompassed in compound (I).

Compound (I) or a salt thereof or a prodrug thereof (hereinafter to be collectively abbreviated as the compound of the present invention) has a GPR40 receptor function regulating action, particularly, a GPR40 receptor agonist activity. Moreover, since the compound of the present invention shows high solubility, low toxicity (e.g., influence on hematological parameters such as red blood cell count, hematocrit value, hemoglobin concentration, MCH, MCHC, MCV, platelet count, leukocyte count, blood reticulocyte count, leukocyte classification and the like; blood biochemical parameters such as total protein, albumin, A/G ratio, glucose, total cholesterol, triglyceride, urea nitrogen, creatinine, total bilirubin, AST, ALT, LDH, ALP, CK, Na, K, Cl, calcium, inorganic phosphorus, retinol (vitamin A) and the like), and a few side effects (e.g., acute toxicity, chronic toxicity, genetic toxicity, reproductive toxicity, cardiotoxicity, drug interaction (CYP inhibitory action), carcinogenicity), it is useful as a safe GPR40 receptor function regulator, preferably, a GPR40 agonist.

The compound of the present invention shows a superior GPR40 receptor function modulating action in mammals (e.g., mouse, rat, hamster, rabbit, cat, dog, bovine, sheep, monkey, human), and are useful as modulators of physiological function in which GPR40 receptor is involved or agents for the prophylaxis or treatment of disease state or disease in which GPR40 receptor is involved.

To be specific, the compound of the present invention is useful as insulin secretion modulators (preferably insulin secretagogues), hypoglycemic drugs and pancreatic β cell protectors.

Particularly, the compound of the present invention is useful as a blood glucose level dependent insulin secretagogue based on its GPR40 receptor agonist activity. In other words, the compound of the present invention is useful as an insulin secretagogue that does not cause hypoglycemia, unlike sulfonylurea.

Furthermore, the compound of the present invention is useful as an agent for the prophylaxis or treatment of diseases including, for example, diabetes, glucose tolerance disorders, ketosis, acidosis, diabetic complications (e.g., diabetic neuropathy, diabetic nephropathy, diabetic retinopathy, macroangiopathy, diabetic gangrene), macular edema, hyperlipidemia, sexual dysfunction, dermatic diseases, arthropathy, osteopenia, arteriosclerosis, thrombotic disease, dyspepsia, deficits in memory and learning, depression, manic-depressive illness, schizophrenia, attention deficit hyperactivity disorder, vision disorder, appetite regulation disorder (e.g., hyperorexia), obesity, hypoglycemia, hypertension, edema, insulin resistance, unstable diabetes, fatty atrophy, insulin allergy, insulinoma, lipotoxicity, hyperinsulinemia, cancer (e.g., breast cancer), metabolic syndrome, immune diseases (e.g., immunodeficiency), inflammatory diseases (e.g., enteritis, arthritis, allergy), multiple sclerosis, acute renal failure and the like. Here, diabetes includes type 1 diabetes, type 2 diabetes, gestational diabetes and obese diabetes. In addition, hyperlipidemia includes hypertriglyceridemia, hypercholesterolemia, hypoHDL-emia, postprandial hyperlipemia and the like.

For diagnostic criteria of diabetes, Japan Diabetes Society reported diagnostic criteria in 1999.

According to this report, diabetes is a condition showing any of a fasting blood glucose level (glucose concentration of intravenous plasma) of not less than 126 mg/dl, a 75 g-oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) and WHO reported diagnostic criteria of diabetes.

According to these reports, diabetes is a condition showing a fasting blood glucose level (glucose concentration of intravenous plasma) of not less than 126 mg/dl or a 75 g oral glucose tolerance test 2 h level (glucose concentration of intravenous plasma) of not less than 200 mg/dl.

In addition, according to the above-mentioned report of ADA and WHO, the impaired glucose tolerance is a condition showing a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 110 mg/dl and less than 126 mg/dl is called IFG (Impaired Fasting Glucose). On the other hand, WHO has defined IFG (Impaired Fasting Glucose) as a condition where the fasting blood sugar level (venous plasma glucose concentration) is not less than 110 mg/dl and less than 126 mg/dl, and calls it IFG (Impaired Fasting Glycemia).

The compound of the present invention can also be used as an agent for the prophylaxis or treatment of diabetes, borderline type, impaired glucose tolerance, IFG (Impaired Fasting Glucose) and IFG (Impaired Fasting Glycemia), as determined according to the above-mentioned diagnostic criteria. Moreover, the compound of the present invention can prevent progress of borderline type, impaired glucose tolerance, IFG (Impaired Fasting Glucose) or IFG (Impaired Fasting Glycemia) into diabetes.

The compound of the present invention is also useful as a therapeutic agent for diabetes with sulfonylurea secondary failure and affords a superior insulin secretion effect and a hypoglycemic effect for diabetic patients for whom sulfonylurea compounds and fast-acting insulin secretagogues fail to provide an insulin secretion effect, and therefore, fail to provide a sufficient hypoglycemic effect.

As the sulfonylurea compound here, a compound having a sulfonylurea skeleton or a derivative thereof (e.g., tolbutamide, glibenclamide, gliclazide, chlorpropamide, tolazamide, acetohexamide, glyclopyramide, glimepiride, glipizide, glybuzole and the like) can be mentioned.

As the fast-acting insulin secretagogue, a compound that promotes insulin secretion from pancreatic β cell in the same manner as a sulfonylurea compound, though it does not have a sulfonylurea skeleton, such as glinide compounds (e.g., repaglinide, senaglinide, nateglinide, mitiglinide, a calcium salt hydrate thereof etc.), and the like, can be mentioned.

The medicament containing the compound of the present invention can be safely administered solely or by mixing with a pharmacologically acceptable carrier according to a method known per se (e.g., the method described in the Japanese Pharmacopoeia etc.) as the production method of a pharmaceutical preparation, and in the form of, for example, tablet (including sugar-coated tablet, film-coated tablet, sublingual tablet, orally disintegrating tablet, buccal and the like), pill, powder, granule, capsule (including soft capsule, microcapsule), troche, syrup, liquid, emulsion, suspension, release control preparation (e.g., immediate-release preparation, sustained-release preparation, sustained-release microcapsule), aerosol, film (e.g., orally disintegrating film, oral mucosa-adhesive film), injection (e.g., subcutaneous injection, intravenous injection, intramuscular injection, intraperitoneal injection), drip infusion, transdermal absorption type preparation, ointment, lotion, adhesive preparation, suppository (e.g., rectal suppository, vaginal suppository), pellet, nasal preparation, pulmonary preparation (inhalant), eye drop and the like orally or parenterally (e.g., intravenous, intramuscular, subcutaneous, intraorgan, intranasal, intradermal, instillation, intracerebral, intrarectal, intravaginal, intraperitoneal and intratumor administrations, administration to the vicinity of tumor, and direct administration to the lesion).

The content of the compound of the present invention in a pharmaceutical preparation is about 0.01 to about 100 wt % of the whole preparation. While the dose varies depending on the subject of administration, administration route, disease, symptom and the like, for example, when it is orally administered to a diabetic patient (body weight about 60 kg), the daily dose of the active ingredient [the compound of the present invention] is about 0.01 to about 30 mg/kg body weight, preferably about 0.1 to about 20 mg/kg body weight, more preferably about 1 to about 20 mg/kg body weight, which may be given at once or in several portions a day (1 to 3 times a day).

As the above-mentioned pharmacologically acceptable carrier, various organic or inorganic carrier substances conventionally used as a preparation material can be mentioned. For example, excipient, lubricant, binder and disintegrant for solid preparations, solvent, solubilizing agents, suspending agent, isotonicity agent, buffer and soothing agent for liquid preparations and the like can be mentioned. Where necessary, additives such as conventional preservatives, antioxidants, colorants, sweetening agents, adsorbing agents, wetting agents and the like can be used.

As the excipient, for example, lactose, sucrose, D-mannitol, starch, corn starch, crystalline cellulose, light anhydrous silicic acid and the like can be mentioned.

As the lubricant, for example, magnesium stearate, calcium stearate, talc, colloidal silica and the like can be mentioned.

As the binder, for example, crystalline cellulose, sucrose, D-mannitol, dextrin, hydroxypropylcellulose, hydroxypropylmethylcellulose, polyvinylpyrrolidone, starch, saccharose, gelatin, methylcellulose, carboxymethylcellulose sodium and the like can be mentioned.

As the disintegrant, for example, starch, carboxymethylcellulose, carboxymethylcellulose calcium, carboxymethylstarch sodium, L-hydroxypropylcellulose and the like can be mentioned.

As the solvent, for example, water for injection, alcohol, propylene glycol, macrogol, sesame oil, corn oil, olive oil and the like can be mentioned.

As the solubilizing agents, for example, polyethylene glycol, propylene glycol, D-mannitol, benzyl benzoate, ethanol, trisaminomethane, cholesterol, triethanolamine, sodium carbonate, sodium citrate and the like can be mentioned.

As the suspending agent, for example, surfactants such as stearyltriethanolamine, sodium lauryl sulfate, lauryl aminopropionic acid, lecithin, benzalkonium chloride, benzethonium chloride, glycerol monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like, and the like can be mentioned.

As the isotonicity agent, for example, glucose, D-sorbitol, sodium chloride, glycerin, D-mannitol and the like can be mentioned.

As the buffer, for example, buffers such as phosphate, acetate, carbonate, citrate and the like, and the like can be mentioned.

As the soothing agent, for example, benzyl alcohol and the like can be mentioned.

As the preservative, for example, p-hydroxybenzoate esters, chlorobutanol, benzyl alcohol, phenethyl alcohol, dehydroacetic acid, sorbic acid and the like can be mentioned.

As the antioxidant, for example, sulfite, ascorbic acid, α-tocopherol and the like can be mentioned.

As the colorant, for example, water-soluble edible tar pigments (e.g., foodcolors such as Food Color Red Nos. 2 and 3, Food Color Yellow Nos. 4 and 5, Food Color Blue Nos. 1 and 2 and the like), water insoluble lake pigments (e.g., aluminum salt of the aforementioned water-soluble edible tar pigment), natural pigments (e.g., β-carotene, chlorophil, ferric oxide red) and the like can be mentioned.

As the sweetening agent, for example, saccharin sodium, dipotassium glycyrrhizinate, aspartame, stevia and the like can be mentioned.

Furthermore, the compound of the present invention can be used in combination with a drug other than the compound of the present invention.

Examples of the drug that can be used in combination with the compound of the present invention (hereinafter sometimes to be abbreviated as concomitant drug) include other therapeutic agent for diabetes, therapeutic agents for diabetic complications, therapeutic agent for hyperlipidemia, antihypertensive agent, antiobesity agent, diuretic, chemotherapeutic agent, immunotherapeutic agent, anti-inflammatory drug, antithrombotic agent, therapeutic agent for osteoporosis, vitamins, anti-dementia, therapeutic drugs for frequent urination or incontinence, therapeutic agent for dysuria and the like. Specifically, the following can be mentioned.

Examples of the therapeutic agent for diabetes include insulin preparations (e.g., animal insulin preparation extracted from pancreas of bovine or swine; human insulin preparation synthesized by genetic engineering using Escherichia coli or yeast; insulin zinc; protamine zinc insulin; insulin fragment or derivative (e.g., INS-1), oral insulin preparation), insulin sensitizers (e.g., pioglitazone or a salt thereof (preferably, hydrochloride), rosiglitazone or a salt thereof (preferably, maleate), metaglidasen, AMG-131, balaglitazone, MBX-2044, rivoglitazone, aleglitazar, chiglitazar, lobeglitazon, PLX-204, PN-2034, GFT-505, THR-0921, compounds described in WO2007/013694, WO2007/018314, WO2008/093639 and WO2008/099794), α-glucosidase inhibitor (e.g., voglibose, acarbose, miglitol, emiglitate), biguanide (e.g., metformin, buformin or a salt thereof (e.g., hydrochloride, fumarate, succinate)), insulin secretagogue (e.g., sulfonylurea (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or calcium salt hydrate thereof), dipeptidyl peptidase IV inhibitor (e.g., alogliptin or a salt thereof (preferably, benzoate), vildagliptin, sitagliptin, saxagliptin, BI1356, GRC8200, MP-513, PF-00734200, PHX1149, SK-0403, ALS2-0426, TA-6666, TS-021, KRP-104, 2-[[6-[(3R)-3-amino-1-piperidinyl]-3,4-dihydro-3-methyl-2,4-dioxo-1(2H)-pyrimidinyl]methyl]-4-fluorobenzonitrile or a salt thereof), β3 agonist (e.g., N-5984), GPR40 agonist (e.g., compounds described in WO2004/041266, WO2004/106276, WO2005/063729, WO2005/063725, WO2005/087710, WO2005/095338, WO2007/013689 and WO2008/001931), GLP-1 receptor agonist (e.g., GLP-1, GLP-1MR agent, liraglutide, exenatide, AVE-0010, BIM-51077, Aib(8,35)hGLP-1 (7,37)NH₂, CJC-1131, albiglutide), amylin agonist (e.g., pramlintide), phosphotyrosine phosphatase inhibitors (e.g., sodium vanadate), gluconeogenesis inhibitor (e.g., glycogen phosphorylase inhibitor, glucose-6-phosphatase inhibitor, glucagon antagonist, FBPase inhibitor), SGLT2 (sodium-glucose cotransporter 2) inhibitor (e.g., Depagliflozin, AVE2268, TS-033, YM543, TA-7284, Remogliflozin, ASP1941), SGLT1 inhibitor, 11β-hydroxysteroid dehydrogenase inhibitor (e.g., BVT-3498, INCB-13739), adiponectin or an agonist thereof, IKK inhibitor (e.g., AS-2868), leptin resistance improving drug, somatostatin receptor agonist, glucokinase activator (e.g., Piragliatin, AZD1656, AZD6370, TTP-355, compounds described in WO2006/112549, WO2007/028135, WO2008/047821, WO2008/050821, WO2008/136428 or WO2008/156757), GIP (Glucose-dependent insulinotropic peptide), GPR119 agonist (e.g., PSN821, MBX-2982, APD597), FGF21, FGF analogue and the like.

Examples of the therapeutic agents for diabetic complications include aldose reductase inhibitors (e.g., tolrestat, epalrestat, zopolrestat, fidarestat, CT-112, ranirestat (AS-3201), lidorestat), neurotrophic factor and a drug for increasing the factor (e.g., NGF, NT-3, BDNF, neurotrophin production-secretion promoter described in WO01/14372 (e.g., 4-(4-chlorophenyl)-2-(2-methyl-1-imidazolyl)-5-[3-(2-methylphenoxy)propyl]oxazole), compound described in WO2004/039365), PKC inhibitor (e.g., ruboxistaurin mesylate), AGE inhibitor (e.g., ALT946, N-phenacylthiazolium bromide (ALT766), EXO-226, pyridorin, pyridoxamine), GABA receptor agonist (e.g., gabapentin, pregabalin), serotonin or noradrenaline reuptake inhibitor (e.g., duloxetine), sodium channel inhibitor (e.g., lacosamide), active oxygen scavenger (e.g., thioctic acid), cerebral vasodilator (e.g., tiapuride, mexiletine), somatostatin receptor agonist (e.g., BIM23190), apoptosis signal regulating kinase-1 (ASK-1) inhibitor and the like.

Examples of the therapeutic agent for hyperlipidemia include HMG-CoA reductase inhibitor (e.g., pravastatin, simvastatin, lovastatin, atorvastatin, fluvastatin, rosuvastatin, pitavastatin or a salt thereof (e.g., sodium salt, calcium salt)), squalene synthase inhibitor (e.g., compound described in WO97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid), fibrate compound (e.g., bezafibrate, clofibrate, simfibrate, clinofibrate), anion exchange resin (e.g., colestyramine), probucol, nicotinic acid drug (e.g., nicomol, niceritrol, niaspan), ethyl icosapentate, phytosterol (e.g., soysterol, γ-oryzanol), cholesterol absorption inhibitor (e.g., zetia), CETP inhibitor (e.g., dalcetrapib, anacetrapib), ω-3 fatty acid preparation (e.g., ω-3-acid ethyl esters 90) and the like.

Examples of the antihypertensive agent include angiotensin converting enzyme inhibitor (e.g., captopril, enalapril, delapril and the like), angiotensin II antagonist (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil and the like), calcium antagonist (e.g., manidipine, nifedipine, amlodipine, efonidipine, nicardipine, amlodipine, cilnidipine and the like), β blocker (e.g., metoprolol, atenolol, propranolol, carvedilol, pindolol and the like), clonidine and the like.

Examples of the antiobesity agent include monoamine uptake inhibitor (e.g., phentermine, sibutramine, mazindol, fluoxetine, tesofensine), serotonin 2C receptor agonist (e.g., lorcaserin), serotonin 6 receptor antagonist, histamine H3 receptor modulator, GABA modulator (e.g., topiramate), neuropeptide Y antagonist (e.g., velneperit), cannabinoid receptor antagonist (e.g., rimonabant, taranabant), ghrelin antagonist, ghrelin receptor antagonist, ghrelin acylation enzyme inhibitor, opioid receptor antagonist (e.g., GSK-1521498), orexin receptor antagonist, melanocortin 4 receptor agonist, 11β-hydroxysteroid dehydrogenase inhibitor (e.g., AZD-4017), pancreatic lipase inhibitor (e.g., orlistat, cetilistat), β3 agonist (e.g., N-5984), diacylglycerol acyltransferase 1 (DGAT1) inhibitor, acetyl CoA carboxylase (ACC) inhibitor, stearoyl-CoA desaturase inhibitor, microsomal triglyceride transfer protein inhibitor (e.g., R-256918), Na-glucose cotransporter inhibitor (e.g., JNJ-28431754, remogliflozin), NFκ inhibitor (e.g., HE-3286), PPAR agonist (e.g., GFT-505, DRF-11605), phosphotyrosine phosphatase inhibitor (e.g., sodium vanadate, Trodusquemin), GPR119 agonist (e.g., PSN-821), glucokinase activator (e.g., AZD-1656), leptin, leptin derivative (e.g., metreleptin), CNTF (ciliary neurotrophic factor), BDNF (brain-derived neurotrophic factor), cholecystokinin agonist, glucagon-like peptide-1 (GLP-1) preparation (e.g., animal GLP-1 preparation extracted from pancreas of bovine, swine; human GLP-1 preparation synthesized by genetic engineering using Escherichia coli, yeast; GLP-1 fragment or derivative (e.g., exenatide, liraglutide)), amylin preparation (e.g., pramlintide, AC-2307), neuropeptide Y agonist (e.g., PYY3-36, PYY3-36 derivative, obineptide, TM-30339, TM-30335), oxyntomodulin preparation: FGF21 preparation (e.g., animal FGF21 preparation extracted from pancreas of bovine, swine; human FGF21 preparation synthesized by genetic engineering using Escherichia coli, yeast; FGF21 fragment or derivative), anorexigenic agent (e.g., P-57) and the like.

Examples of the diuretics include xanthine derivatives (e.g., sodium salicylate and theobromine, calcium salicylate and theobromine etc.), thiazide preparations (e.g., ethiazide, cyclopenthiazide, trichloromethiazide, hydrochlorothiazide, hydroflumethiazide, bentylhydrochlorothiazide, penflutiazide, poly5thiazide, methyclothiazide etc.), antialdosterone preparations (e.g., spironolactone, triamterene etc.), carbonate dehydratase inhibitors (e.g., acetazolamide and the like), chlorobenzenesulfonamide preparations (e.g., chlortalidone, mefruside, indapamide etc.), azosemide, isosorbide, etacrynic acid, piretanide, bumetanide, furosemide and the like.

Examples of the chemotherapeutic agent include alkylating agents (e.g., cyclophosphamide, ifosfamide), metabolic antagonists (e.g., methotrexate, 5-fluorouracil), antitumor antibiotics (e.g., mitomycin, adriamycin), plant-derived antitumor agent (e.g., vincristine, vindesine, Taxol), cisplatin, carboplatin, etoposide and the like. Of these, Furtulon or NeoFurtulon, which are 5-fluorouracil derivatives, and the like are preferable.

Examples of the immunotherapeutic agent include microorganism or bacterial components (e.g., muramyl dipeptide derivative, Picibanil), polysaccharides having immunity potentiating activity (e.g., lentinan, schizophyllan, krestin), cytokines obtained by genetic engineering techniques (e.g., interferon, interleukin (IL)), colony stimulating factors (e.g., granulocyte colony stimulating factor, erythropoietin) and the like, with preference given to interleukins such as IL-1, IL-2, IL-12 and the like.

Examples of the antiinflammatory agent include non-steroidal antiinflammatory agents such as aspirin, acetaminophen, indomethacin and the like.

Examples of the antithrombotic agent include heparin (e.g., heparin sodium, heparin calcium, enoxaparin sodium, dalteparin sodium), warfarin (e.g., warfarin potassium), anti-thrombin drugs (e.g., aragatroban, dabigatran), FXa inhibitors (e.g., rivaroxaban, apixaban, edoxaban, YM150, the compounds described in WO002/06234, WO2004/048363, WO2005/030740, WO2005/058823 and WO2005/113504), thrombolytic agents (e.g., urokinase, tisokinase, alteplase, nateplase, monteplase, pamiteplase), platelet aggregation inhibitors (e.g., ticlopidine hydrochloride, clopidogrel, prasugrel, E5555, SHC530348, cilostazol, ethyl icosapentate, beraprost sodium, sarpogrelate hydrochloride) and the like.

Examples of the therapeutic agent for osteoporosis include alfacalcidol, calcitriol, elcatonin, calcitonin salmon, estriol, ipriflavone, pamidronate disodium, alendronate sodium hydrate, incadronate disodium, risedronate disodium and the like.

Examples of the vitamin include vitamin B₁, vitamin B₁₂ and the like.

Examples of the antidementia agent include tacrine, donepezil, rivastigmine, galanthamine and the like.

Examples of the therapeutic agent for pollakiuria or urinary incontinence include flavoxate hydrochloride, oxybutynin hydrochloride, propiverine hydrochloride and the like.

Examples of the therapeutic agent for dysuria include acetylcholine esterase inhibitors (e.g., distigmine) and the like.

Furthermore, drugs having a cachexia-ameliorating action established in animal models and clinical situations, such as cyclooxygenase inhibitors (e.g., indomethacin), progesterone derivatives (e.g., megestrol acetate), glucosteroids (e.g., dexamethasone), metoclopramide agents, tetrahydrocannabinol agents, fat metabolism improving agents (e.g., eicosapentanoic acid), growth hormones, IGF-1, or antibodies to a cachexia-inducing factor such as TNF-α, LIF, IL-6, oncostatin M and the like, can be used in combination with the compound of the present invention.

Furthermore, glycosylation inhibitors (e.g., ALT-711), nerve regeneration promoting drugs (e.g., Y-128, VX853, prosaptide), antidepressants (e.g., desipramine, amitriptyline, imipramine, etc.), antiepileptics (e.g., lamotrigine, Trileptal, Keppra, Zonegran, Pregabalin, Harkoseride and carbamazepine), antiarrhythmic agents (e.g., mexiletine), acetylcholine receptor ligands (e.g., ABT-594), endothelin receptor antagonists (e.g., ABT-627), monoamine uptake inhibitors (e.g., tramadol), narcotic analgesics (e.g., morphine), GABA receptor agonists (e.g., gabapentin and gabapentin MR agents), α2 receptor agonists (e.g., clonidine), local analgesics (e.g., capsaicin), antianxiety drugs (e.g., benzothiazepines), phosphodiesterase inhibitors (e.g., sildenafil), dopamine receptor agonists (e.g., apomorphine), midazolam, ketoconazole and the like can also be used in combination with the compound of the present invention.

The concomitant drug is preferably an insulin preparation, a PPAR function regulator (preferably, pioglitazone or hydrochloride thereof), an α-glucosidase inhibitor (preferably, voglibose), biguanide (preferably metformin or hydrochloride thereof), sulfonylurea (preferably glibenclamide, glimepiride), mitiglinide or calcium salt hydrate thereof, nateglinide, dipeptidyl peptidase IV inhibitor (preferably alogliptin or benzoate thereof, 2-[[6-[(3R)-3-amino-1-piperidinyl]-3,4-dihydro-3-methyl-2,4-dioxo-1(2H)-pyrimidinyl]methyl]-4-fluorobenzonitrile or succinate thereof, 2-[2-(3-(R)-amino-piperidin-1-yl)-5-fluoro-6-oxo-6H-pyrimidin-1-ylmethyl]-benzonitrile or tartrate thereof) and the like.

A combination of the compound of the present invention and a concomitant drug affords the following effects:

(1) the dose can be reduced as compared to a single administration of the compound of the present invention or a concomitant drug,
(2) a long treatment period can be set by selecting a concomitant drug having a different mechanism of action from that of the compound of the present invention,
(3) a sustained treatment effect can be designed by selecting a concomitant drug having a different mechanism of action from that of the compound of the present invention,
(4) a synergistic effect can be afforded by a combined use of the compound of the present invention and a concomitant drug, and the like.

When the compound of the present invention and a concomitant drug are used in combination, the administration time of the compound of the present invention and the concomitant drug is not restricted, and the compound of the present invention and the concomitant drug can be administered to an administration subject simultaneously, or may be administered at staggered times. The dosage of the concomitant drug may be determined according to the dose clinically used, and can be appropriately selected depending on the administration subject, administration route, disease, combination and the like.

As the administration mode of the compound of the present invention and a concomitant drug, the following methods can be mentioned: (1) administration of a single preparation obtained by simultaneous formulation of the compound of the present invention and a concomitant drug, (2) simultaneous administration by the same administration route of two kinds of preparations obtained by separate formulation of the compound of the present invention and a concomitant drug, (3) administration by the same administration route at staggered times of two kinds of preparations obtained by separate formulation of the compound of the present invention and a concomitant drug, (4) simultaneous administration by different administration routes of two kinds of preparations obtained by separate formulation of the compound of the present invention and a concomitant drug, (5) administration by different administration routes at staggered times of two kinds of preparations obtained by separate formulation of the compound of the present invention and a concomitant drug (e.g., the compound of the present invention and a concomitant drug are administered in this order, or in the reverse order), and the like.

### EXAMPLES

The present invention is further explained in detail by referring to the following Reference Examples, Examples, Formulation Examples and Experimental Example, which are mere working examples not to be construed as limitative and may be changed without departing from the scope of the present invention. The term “room temperature” in the following Reference Examples and Examples indicates the range of generally from about 10° C. to about 35° C. As for “%”, the yield is in mol/mol %, the solvent used for chromatography is in % by volume and other “%” is in % by weight. OH proton, NH proton etc. that could not be confirmed due to broad peak by proton NMR spectrum are not included in the data.

Other symbols used herein mean the following:

s: singlet
d: doublet
t: triplet
q: quartet
m: multiplet
br: broad
J: coupling constant

### Hz: Hertz

CDCl₃: deuterated chloroform
DMSO-d₆: deuterated dimethyl sulfoxide
¹H NMR: proton nuclear magnetic resonance

In the following Reference Examples and Examples, mass spectrum (MS), nuclear magnetic resonance spectrum (NMR) and melting point were measured using the following apparatuses. MS measurement device: Waters ZMD, Waters ZQ2000 or Agilent Technologies, Agilent G6100 series. Ionization method: electron impact ionization method (Electron Spray Ionization: ESI), or atmospheric pressure chemical ionization method (Atmospheric Pressure Chemical Ionization: APCI). Unless otherwise specified, ESI was used.

NMR measurement device: Varian, Varian Gemini 200 (200 MHz), Varian Gemini 300 (300 MHz), Bruker•BioSpin K.K. AVANCE 300.

In the present specification, the melting point (m.p.) refers to that measured using, for example, differential scanning calorimeter EXSTER6000 (DSC6200) manufactured by SII NanoTechnology Inc. or micromelting point measuring apparatus (Büchi, B-545) and the like.

In general, melting points vary depending on measurement apparatuses, measurement conditions and the like. The crystal in the present specification may show a different melting point from that described in the present specification, as long as it is within general error range.

### Reference Example 1

### methyl [(3S)-6-amino-2,3-dihydro-1-benzofuran-3-yl]acetate

To a solution of methyl [(3S)-6-hydroxy-2,3-dihydro-1-benzofuran-3-yl]acetate (41.6 g, 200 mmol) in pyridine (400 mL) was added dropwise under ice-cooling trifluoromethanesulfonic anhydride (40.4 mL, 240 mmol), and the mixture was stirred at room temperature overnight. The reaction mixture was concentrated under reduced pressure, diluted with ethyl acetate, washed with 1 M hydrochloric acid and saturated brine, dried over anhydrous magnesium sulfate, and concentrated under reduced pressure. The residue was purified by silica gel column chromatography (hexane:ethyl acetate=95:5-70:30) to give methyl [(3S)-6-{[(trifluoromethyl)sulfonyl]oxy}-2,3-dihydro-1-benzofuran-3-yl]acetate (67.3 g) as an oil. To a solution of the obtained oil (67.3 g) and benzophenoneimine (46.5 mL, 277 mmol) and cesium carbonate (194 g, 594 mmol) in tetrahydrofuran (660 mL) were added under an argon atmosphere tris(dibenzylideneacetone)dipalladium (0) (5.44 g, 5.94 mmol) and 4,5-bis(diphenylphosphino)-9,9-dimethylxanthene (6.87 g, 11.9 mmol), and the mixture was stirred at 80° C. overnight. The reaction mixture was cooled to room temperature, filtered through celite, and concentrated under reduced pressure to give a brown oil. To a solution of the obtained oil in tetrahydrofuran (400 mL) was added 3 M hydrochloric acid (330 mL, 990 mmol), and the mixture was stirred at room temperature for 2 hr. The reaction mixture was concentrated under reduced pressure, diluted with distilled water, and washed with ethyl acetate. The aqueous layer was neutralized with aqueous sodium hydroxide solution and extracted with ethyl acetate. The extract was washed with saturated brine, dried over anhydrous magnesium sulfate, and concentrated under reduced pressure. The residue was purified by silica gel column chromatography (hexane:ethyl acetate=95:5-60:40) to give an oil. The obtained oil compound was crystallized from diisopropyl ether (10 mL) and heptane (50 mL), and the crystals were collected by filtration and washed with heptane to give the title compound (20.6 g, yield 50%) as a white solid.

MS m/z 208 (M+H)⁺.

### Reference Example 2

### methyl [(3S)-6-{[(2-nitrophenyl)sulfonyl]amino}-2,3-dihydro-1-benzofuran-3-yl]acetate

To a solution of methyl [(3S)-6-amino-2,3-dihydro-1-benzofuran-3-yl]acetate (3.00 g, 14.5 mmol) in pyridine (24 mL) was added under ice-cooling 2-nitrobenzenesulfonyl chloride (3.92 g, 15.9 mmol), and the mixture was stirred at room temperature for 16 hr. The mixture was concentrated under reduced pressure to remove pyridine, and 1 M hydrochloric acid and ethyl acetate was added. Insoluble material was filtered off through celite, and the filtrate was extracted with ethyl acetate. The extract was washed with saturated brine, dried over anhydrous sodium sulfate, and concentrated under reduced pressure. The residue was purified by silica gel column chromatography (hexane:ethyl acetate=90:10-50:50) to give a yellow solid. The obtained solid was triturated with diisopropyl ether-ethyl acetate, and washed with diisopropyl ether to give the title compound (4.86 g, yield 86%) as a yellow solid.

MS m/z 393 (M+H)⁺.

### Reference Example 3

### methyl [(3S)-6-{[(3-bromo-2-hydroxyphenyl)methylidene]amino}-2,3-dihydro-1-benzofuran-3-yl]acetate

A solution of 3-bromo-2-hydroxybenzaldehyde (85.5 g, 425 mmol) and methyl [(3S)-6-amino-2,3-dihydro-1-benzofuran-3-yl]acetate (88.1 g, 425 mmol) in toluene (425 mL) was stirred overnight at room temperature. The reaction mixture was concentrated under reduced pressure to give the title compound (yield 100%) as an orange solid.

MS m/z 390 (M+H)⁺.

### Reference Example 4

### methyl {(3S)-6-[(7-bromo-2,3-dihydro-1-benzofuran-3-yl)amino]-2,3-dihydro-1-benzofuran-3-yl}acetate

To a solution (15-20° C.) of trimethylsulfoxonium iodide (187 g, 850 mmol) in dimethyl sulfoxide (1.0 L) was slowly added sodium hydride (50-72% oil, 25.0 g) under a nitrogen stream, and the mixture was stirred at 15-20° C. for 1 hr. Thereto was slowly added dropwise (15-20° C.) a solution of methyl [(3S)-6-{[(3-bromo-2-hydroxyphenyl)methylidene]amino}-2,3-dihydro-1-benzofuran-3-yl]acetate (425 mmol) in dimethyl sulfoxide (500 mL), and the mixture was stirred for 2 hr. To the reaction mixture was added aqueous ammonium chloride solution (100 mL), and the mixture was diluted with ethyl acetate and stirred at room temperature. The reaction mixture was washed with water and saturated brine, dried over anhydrous sodium sulfate, and concentrated under reduced pressure. The residue was purified by silica gel column chromatography (hexane:ethyl acetate=85:15-75:25) to give the title compound (118.0 g, yield 69%) as a yellow oil.

¹H NMR (300 MHz, CDCl₃) δ 2.55 (1H, dd, J=16.3, 9.1 Hz), 2.74 (1H, dd, J=16.3, 5.7 Hz), 3.72 (3H, s), 3.73-3.85 (1H, m), 3.97 (1H, d, J=8.0 Hz), 4.25 (1H, dd, J=9.5, 6.1 Hz), 4.48 (1H, dd, J=9.8, 4.2 Hz), 4.68-4.84 (2H, m), 5.20-5.31 (1H, m), 6.08-6.16 (2H, m), 6.76-6.85 (1H, m), 6.97 (1H, d, J=8.3 Hz), 7.28 (1H, d, J=7.6 Hz), 7.41 (1H, d, J=7.6 Hz).

### Reference Example 5

### methyl {(3S)-6-[(7-bromo-2,3-dihydro-1-benzofuran-3-yl)(trifluoroacetyl)amino]-2,3-dihydro-1-benzofuran-3-yl}acetate

A solution of methyl {(3S)-6-[(7-bromo-2,3-dihydro-1-benzofuran-3-yl)amino]-2,3-dihydro-1-benzofuran-3-yl}acetate (118.0 g, 292 mmol) and triethylamine (44.3 g, 438 mmol) in tetrahydrofuran (730 mL) was ice-cooled, and trifluoroacetic anhydride (73.5 g, 350 mmol) was added dropwise (5-10° C.). The reaction mixture was stirred at 5-10° C. for 1 hr, concentrated under reduced pressure, and diluted with ethyl acetate. The solution was washed with aqueous sodium bicarbonate solution and saturated brine, dried over anhydrous magnesium sulfate, and concentrated under reduced pressure. The residue was purified by silica gel column chromatography (hexane:ethyl acetate=85:15-70:30) to give the title compound (145.0 g, yield 99%) as a yellow oil.

¹H NMR (300 MHz, CDCl₃) δ 2.49-2.64 (1H, m), 2.66-2.83 (1H, m), 3.71 (3H, s), 3.77-3.89 (1H, m), 4.18-4.34 (1H, m), 4.59-4.86 (3H, m), 5.91-6.04 (1H, m), 6.36-6.49 (1H, m), 6.56-6.73 (1H, m), 6.76-6.85 (1H, m), 6.86-7.13 (1H, m), 7.27-7.41 (2H, m).

### Reference Example 6

### methyl [(3S)-6-{[(3R)-7-bromo-2,3-dihydro-1-benzofuran-3-yl](trifluoroacetyl)amino}-2,3-dihydro-1-benzofuran-3-yl]acetate

Methyl {(3S)-6-[(7-bromo-2,3-dihydro-1-benzofuran-3-yl)(trifluoroacetyl)amino]-2,3-dihydro-1-benzofuran-3-yl}acetate (10.3 g) was separated by HPLC to give the title compound (4.5 g, >99% d.e., recovery rate 87%) as a yellow oil.

¹H NMR (300 MHz, CDCl₃) δ 2.47-2.65 (1H, m), 2.66-2.82 (1H, m), 3.64-3.93 (4H, m), 4.16-4.36 (1H, m), 4.58-4.86 (3H, m), 5.91-6.02 (1H, m), 6.34-6.50 (1H, m), 6.56-7.15 (3H, m), 7.28-7.41 (2H, m).

(high performance liquid chromatography conditions) column: CHIRALPAK AD (manufactured by Daicel Chemical Industries, Ltd.)
mobile phase: hexane/isopropyl alcohol (volume ratio: 90/10)
flow rate: 80 mL/min
detection: UV (220 nm)
temperature: 30° C.
retention time: 35 min

### Reference Example 7

### methyl [(3S)-6-{[(3S)-7-bromo-2,3-dihydro-1-benzofuran-3-yl](trifluoroacetyl)amino}-2,3-dihydro-1-benzofuran-3-yl]acetate

Methyl {(3S)-6-[(7-bromo-2,3-dihydro-1-benzofuran-3-yl)(trifluoroacetyl)amino]-2,3-dihydro-1-benzofuran-3-yl}acetate (10.3 g) was separated by HPLC to give the title compound (4.38 g, 99.8% d.e., recovery rate 85%) as a white solid.

¹H NMR (300 MHz, CDCl₃) δ 2.57 (1H, dd, J=16.7, 9.1 Hz), 2.65-2.82 (1H, m), 3.60-3.94 (4H, m), 4.14-4.38 (1H, m), 4.54-4.87 (3H, m), 5.89-6.07 (1H, m), 6.35-6.49 (1H, m), 6.56-7.18 (3H, m), 7.28-7.45 (2H, m).

(high performance liquid chromatography conditions) column: CHIRALPAK AD (manufactured by Daicel Chemical Industries, Ltd.)
mobile phase: hexane/isopropyl alcohol (volume ratio: 90/10)
flow rate: 80 mL/min
detection: UV (220 nm)
temperature: 30° C.
retention time: 43 min

Alternatively, to methyl {(3S)-6-[(7-bromo-2,3-dihydro-1-benzofuran-3-yl)(trifluoroacetyl)amino]-2,3-dihydro-1-benzofuran-3-yl}acetate (145 g) was added diisopropyl ether (1.0 L), and the mixture was stirred at 95° C. for 1 hr. The mixture was allowed to cool to precipitate a white solid, which was collected by filtration. The obtained solid was recrystallized from diisopropyl ether (1.0 L) to give the title compound (40.6 g, 99.9% d.e.) as a white solid. The obtained crystals were applied to single crystal automatic X ray structure analyzer R-AXIS RAPID (manufactured by Rigaku Corporation, measurement temperature: −173° C.) to determine the absolute configuration of the title compound as S, S.

### Reference Example 8

### methyl {(3S)-6-[{(3S)-7-[(2R,6S)-2,6-dimethylmorpholin-4-yl]-2,3-dihydro-1-benzofuran-3-yl}(trifluoroacetyl)amino]-2,3-dihydro-1-benzofuran-3-yl}acetate

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (200 mg, 0.400 mmol), (2R,6S)-2,6-dimethylmorpholine (98.5 μL, 0.800 mmol) and cesium carbonate (261 mg, 0.800 mmol) in toluene (2 mL) were added under an argon atmosphere tris(dibenzylideneacetone)dipalladium (0) (14.7 mg, 0.016 mmol) and 4,5-bis(diphenylphosphino)-9,9-dimethylxanthene (18.5 mg, 0.032 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give the title compound (168 mg, yield 79%) as a yellow oil. MS m/z 535 (M+H)⁺.

### Reference Example 9

### methyl [(3S)-6-{[(3S)-7-(piperidin-1-yl)-2,3-dihydro-1-benzofuran-3-yl](trifluoroacetyl)amino}-2,3-dihydro-1-benzofuran-3-yl]acetate

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (200 mg, 0.400 mmol), piperidine (79.3 μL, 0.800 mmol) and cesium carbonate (261 mg, 0.800 mmol) in toluene (2 mL) were added under an argon atmosphere tris(dibenzylideneacetone)dipalladium (0) (14.7 mg, 0.016 mmol) and (R)-(+)-2,2′-bis(diphenylphosphino)-1,1′-binaphthyl (19.9 mg, 0.032 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give the title compound (67.5 mg, yield 33%) as a pale-yellow oil.

MS m/z 505 (M+H)⁺.

### Reference Example 10

### methyl [(3S)-6-{[(3S)-7-(2,6-dimethyl-4-{[(2S)-5-oxopyrrolidin-2-yl]methoxy}phenyl)-2,3-dihydro-1-benzofuran-3-yl](trifluoroacetyl)amino}-2,3-dihydro-1-benzofuran-3-yl]acetate

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (6.00 g, 12.0 mmol), [4-(methoxymethoxy)-2,6-dimethylphenyl]boronic acid (3.02 g, 14.4 mmol) and 2 M aqueous sodium carbonate solution (18.0 mL, 36.0 mmol) in toluene (40 mL) were added under an argon atmosphere tris(dibenzylideneacetone)dipalladium (0) (439 mg, 0.480 mmol) and dicyclohexyl(2′,6′-dimethoxybiphenyl-2-yl)phosphane (788 mg, 1.92 mmol) and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give a yellow oil (7.65 g). To a solution of the obtained oil (7.65 g) in methanol (40 mL) was added 10% hydrogen chloride containing methanol solution (3.8 mL), and the mixture was stirred at 40° C. for 2 hr. The reaction mixture was neutralized with saturated aqueous sodium hydrogen carbonate, diluted with distilled water and extracted with ethyl acetate. The extract was washed with saturated brine, dried over anhydrous magnesium sulfate, and concentrated under reduced pressure to give a solid. This was triturated with hexane-ethyl acetate to give methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate (6.47 g) as a white solid. To a solution of the obtained white solid (3.00 g, 5.54 mmol) and [(2S)-5-oxopyrrolidin-2-yl]methyl 4-methylbenzenesulfonate (1.94 g, 7.20 mmol) in N,N-dimethylformamide (18 mL) was added tripotassium phosphate (1.76 g, 8.31 mmol), and the mixture was stirred at 80° C. for 3 hr. Then, [(2S)-5-oxopyrrolidin-2-yl]methyl 4-methylbenzenesulfonate (448 mg) and tripotassium phosphate (470 mg) were added, and the mixture was stirred at 80° C. for 1 hr. The reaction mixture was neutralized with aqueous ammonium chloride solution, diluted with distilled water, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-20:80) to give the title compound (2.92 g, yield 82%) as a white solid.

MS m/z 639 (M+H)⁺.

### Reference Example 11

### methyl {(3S)-6-[{7-[(2-aminophenyl)amino]-2,3-dihydro-1-benzofuran-3-yl}(trifluoroacetyl)amino]-2,3-dihydro-1-benzofuran-3-yl}acetate

To a solution of methyl {(3S)-6-[(7-bromo-2,3-dihydro-1-benzofuran-3-yl)(trifluoroacetyl)amino]-2,3-dihydro-1-benzofuran-3-yl}acetate (2.00 g, 4.00 mmol), 2-nitroaniline (607 mg, 4.40 mmol) and tripotassium phosphate (1.70 g, 8.00 mmol) in toluene (20 mL) were added under an argon atmosphere tris(dibenzylideneacetone)dipalladium (0) (183 mg, 0.200 mmol) and dicyclohexyl[2′,4′,6′-tri(propan-2-yl)biphenyl-2-yl]phosphane (191 mg, 0.400 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-67:33) to give an orange solid (2.26 g). The obtained solid (2.26 g) was dissolved in methanol (20 mL) and tetrahydrofuran (2 mL), 10% palladium-carbon (50% water-containing product, 226 mg) was added, and the mixture was stirred under a hydrogen atmosphere (balloon pressure) at room temperature for 5 hr. The catalyst was filtered off, and the filtrate was concentrated under reduced pressure. The residue was purified by silica gel column chromatography (hexane:ethyl acetate=95:5-60:40) to give the title compound (1.97 g, yield 93%) as a yellow solid.

MS m/z 528 (M+H)⁺.

### Reference Example 12

### methyl [(3S)-6-{[7-(2-methyl-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{7-[(2-aminophenyl)amino]-2,3-dihydro-1-benzofuran-3-yl}(trifluoroacetyl)amino]-2,3-dihydro-1-benzofuran-3-yl}acetate (1.97 g, 3.73 mmol) and acetic anhydride (2.82 mL, 29.9 mmol) in acetic acid (12 mL) was stirred overnight at 130° C. The reaction mixture was cooled to room temperature, concentrated under reduced pressure to remove acetic acid, neutralized with saturated aqueous sodium hydrogen carbonate, and diluted with distilled water, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) and HPLC to give the title compound (1.52 g, yield 74%) as a white solid.

MS m/z 552 (M+H)⁺.

### Reference Example 13

### methyl [(3S)-6-{[(3S)-7-(2-methyl-1H-benzimidazol-1-yl)-2,3-dihydro-1-benzofuran-3-yl](trifluoroacetyl)amino}-2,3-dihydro-1-benzofuran-3-yl]acetate

Methyl [(3S)-6-{[7-(2-methyl-1H-benzimidazol-1-yl)-2,3-dihydro-1-benzofuran-3-yl](trifluoroacetyl)amino}-2,3-dihydro-1-benzofuran-3-yl]acetate (1.51 g) was resolved by HPLC to give the title compound (647 mg, recovery rate 86%) as a white solid.

MS m/z 552 (M+H)⁺.

(high performance liquid chromatography conditions)
column: CHIRALPAK IC (manufactured by Daicel Chemical Industries, Ltd.)
mobile phase: hexane/ethanol/diethylamine (volume ratio: 500/500/1)
flow rate: 60 mL/min
detection: UV (220 nm)
temperature: 40° C.
retention time: 11.4 min (>99%).

### Reference Example 14

### methyl [(3S)-6-{[(3R)-7-(2-methyl-1H-benzimidazol-1-yl)-2,3-dihydro-1-benzofuran-3-yl](trifluoroacetyl)amino}-2,3-dihydro-1-benzofuran-3-yl]acetate

Methyl [(3S)-6-{[7-(2-methyl-1H-benzimidazol-1-yl)-2,3-dihydro-1-benzofuran-3-yl](trifluoroacetyl)amino}-2,3-dihydro-1-benzofuran-3-yl]acetate (1.51 g) was resolved by HPLC to give the title compound (684 mg, recovery rate 90%) as a solid.

MS m/z 552 (M+H)⁺.

(high performance liquid chromatography conditions)
column: CHIRALPAK IC (manufactured by Daicel Chemical Industries, Ltd.)
mobile phase: hexane/ethanol/diethylamine (volume ratio: 500/500/1)
flow rate: 60 mL/min
detection: UV (220 nm)
temperature: 40° C.
retention time: 13.8 min (98%)

### Reference Example 15

### methyl {(3S)-6-[{(3R)-7-[(2-aminophenyl)amino]-2,3-dihydro-1-benzofuran-3-yl}(trifluoroacetyl)amino]-2,3-dihydro-1-benzofuran-3-yl}acetate

To a solution of methyl [(3S)-6-{[(3R)-7-bromo-2,3-dihydro-1-benzofuran-3-yl](trifluoroacetyl)amino}-2,3-dihydro-1-benzofuran-3-yl]acetate (1.00 g, 2.00 mmol), 2-nitroaniline (304 mg, 2.20 mmol) and tripotassium phosphate (849 mg, 4.00 mmol) in toluene (10 mL) were added under an argon atmosphere tris(dibenzylideneacetone)dipalladium (0) (91.6 mg, 0.100 mmol) and dicyclohexyl[2′,4′,6′-tri(propan-2-yl)biphenyl-2-yl]phosphane (95.3 mg, 0.200 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give an orange non-crystalline powder (1.19 g). The obtained non-crystalline powder (1.19 g) was dissolved in methanol (10 mL), 10% palladium-carbon (50% water-containing product, 120 mg) was added, and the mixture was stirred under a hydrogen atmosphere (balloon pressure) at room temperature for 2 hr. The catalyst was filtered off, and the filtrate was concentrated under reduced pressure. The residue was purified by silica gel column chromatography (hexane:ethyl acetate=95:5-60:40) to give the title compound (973 mg, yield 92%) as a white solid.

MS m/z 528 (M+H)⁺.

### Reference Example 16

### methyl [(3S)-6-{[(3R)-7-(2-ethyl-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3R)-7-[(2-aminophenyl)amino]-2,3-dihydro-1-benzofuran-3-yl}(trifluoroacetyl)amino]-2,3-dihydro-1-benzofuran-3-yl}acetate (300 mg, 0.569 mmol) in N,N-dimethylacetamide (3 mL) was ice-cooled, and propanoyl chloride (59.6 μL, 0.682 mmol) was added dropwise. The reaction mixture was stirred at room temperature for 20 min, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (2 mL) was stirred overnight at 140° C. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) to give the title compound (231 mg, yield 72%) as a white solid.

MS m/z 566 (M+H)⁺.

### Reference Example 17

### methyl [(3S)-6-{[(3R)-7-(2-ethoxy-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3R)-7-[(2-aminophenyl)amino]-2,3-dihydro-1-benzofuran-3-yl}(trifluoroacetyl)amino]-2,3-dihydro-1-benzofuran-3-yl}acetate (300 mg, 0.569 mmol) and tetraethoxymethane (357 μL, 1.71 mmol) in acetic acid (3 mL) was stirred at 60° C. for 30 min. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over-anhydrous magnesium sulfate, and concentrated under reduced pressure. The residue was purified by silica gel column chromatography (hexane:ethyl acetate=95:5-40:60) to give the title compound (284 mg, yield 86%) as a colorless oil.

MS m/z 582 (M+H)⁺.

### Reference Example 18

### methyl {(3S)-6-[{(3R)-7-[(5-fluoro-2-nitrophenyl)amino]-2,3-dihydro-1-benzofuran-3-yl}(trifluoroacetyl)amino]-2,3-dihydro-1-benzofuran-3-yl}acetate

To a solution of methyl [(3S)-6-{[(3R)-7-bromo-2,3-dihydro-1-benzofuran-3-yl](trifluoroacetyl)amino}-2,3-dihydro-1-benzofuran-3-yl]acetate (1.00 g, 2.00 mmol) and 5-fluoro-2-nitroaniline (343 mg, 2.20 mmol) and tripotassium phosphate (849 mg, 4.00 mmol) in toluene (10 mL) were added under an argon atmosphere tris(dibenzylideneacetone)dipalladium (0) (91.6 mg, 0.100 mmol) and dicyclohexyl[2′,4′,6′-tri(propan-2-yl)biphenyl-2-yl]phosphane (95.3 mg, 0.200 mmol) and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give the title compound (1.16 g, yield 100%) as an orange non-crystalline powder.

MS m/z 574 (M−H)⁻.

### Reference Example 19

### methyl {(3S)-6-[{(3R)-7-[(2-amino-5-fluorophenyl)amino]-2,3-dihydro-1-benzofuran-3-yl}(trifluoroacetyl)amino]-2,3-dihydro-1-benzofuran-3-yl}acetate

Methyl {(3S)-6-[{(3R)-7-[(5-fluoro-2-nitrophenyl)amino]-2,3-dihydro-1-benzofuran-3-yl}(trifluoroacetyl)amino]-2,3-dihydro-1-benzofuran-3-yl}acetate (2.00 mmol) was dissolved in methanol (10 mL), 10% palladium-carbon (50% water-containing product, 120 mg) was added, and the mixture was stirred under a hydrogen atmosphere (balloon pressure) at room temperature for 2 hr. The catalyst was filtered off, and the filtrate was concentrated under reduced pressure. The residue was purified by silica gel column chromatography (hexane:ethyl acetate=95:5-60:40) to give the title compound (935 mg, yield 86%) as a pale-pink solid.

MS m/z 546 (M+H)⁺.

### Reference Example 20

### methyl [(3S)-6-{[(3R)-7-(2-ethyl-6-fluoro-1H-benzimidazol-1-yl)-2,3-dihydro-1-benzofuran-3-yl](trifluoroacetyl)amino}-2,3-dihydro-1-benzofuran-3-yl]acetate

In the same manner as in Reference Example 16, the title compound (249 mg, yield 78%) was obtained as a white solid from methyl {(3S)-6-[{(3R)-7-[(2-amino-5-fluorophenyl)amino]-2,3-dihydro-1-benzofuran-3-yl}(trifluoroacetyl)amino]-2,3-dihydro-1-benzofuran-3-yl}acetate (300 mg, 0.550 mmol) and propanoyl chloride (57.6 μL, 0.660 mmol).

MS m/z 584 (M+H)⁺.

### Reference Example 21

### methyl [(3S)-6-{[(3R)-7-(2-ethoxy-6-fluoro-1H-benzimidazol-1-yl)-2,3-dihydro-1-benzofuran-3-yl](trifluoroacetyl)amino}-2,3-dihydro-1-benzofuran-3-yl]acetate

In the same manner as in Reference Example 17, the title compound (281 mg, yield 85%) was obtained as a white solid from methyl {(3S)-6-[{(3R)-7-[(2-amino-5-fluorophenyl)amino]-2,3-dihydro-1-benzofuran-3-yl}(trifluoroacetyl)amino]-2,3-dihydro-1-benzofuran-3-yl}acetate (300 mg, 0.550 mmol) and tetraethoxymethane (345 μL, 1.65 mmol).

MS m/z 600 (M+H)⁺.

### Reference Example 22

### methyl {(3S)-6-[{(3S)-7-[(2-aminophenyl)amino]-2,3-dihydro-1-benzofuran-3-yl)}(trifluoroacetyl)amino]-2,3-dihydro-1-benzofuran-3-yl}acetate

In the same manner as in Reference Example 15, the title compound (553 mg, yield 100%) was obtained as pink crystals from methyl [(3S)-6-{[(3S)-7-bromo-2,3-dihydro-1-benzofuran-3-yl](trifluoroacetyl)amino}-2,3-dihydro-1-benzofuran-3-yl]acetate (500 mg, 1.00 mmol) and 2-nitroaniline (152 mg, 1.10 mmol).

MS m/z 528 (M+H)⁺.

### Reference Example 23

### methyl [(3S)-6-{[(3S)-7-(2-ethyl-1H-benzimidazol-1-yl)-2,3-dihydro-1-benzofuran-3-yl](trifluoroacetyl)amino}-2,3-dihydro-1-benzofuran-3-yl]acetate

In the same manner as in Reference Example 16, the title compound (177 mg, yield 100%) was obtained as a white solid from methyl {(3S)-6-[{(3S)-7-[(2-aminophenyl)amino]-2,3-dihydro-1-benzofuran-3-yl}(trifluoroacetyl)amino]-2,3-dihydro-1-benzofuran-3-yl}acetate (160 mg, 0.303 mmol) and propanoyl chloride (31.8 μL, 0.364 mmol).

MS m/z 566 (M+H)⁺.

### Reference Example 24

### methyl [(3S)-6-{[(3S)-7-(2-ethoxy-1H-benzimidazol-1-yl)-2,3-dihydro-1-benzofuran-3-yl](trifluoroacetyl)amino}-2,3-dihydro-1-benzofuran-3-yl]acetate

In the same manner as in Reference Example 17, the title compound (163 mg, yield 92%) was obtained as a white solid from methyl {(3S)-6-[{(3S)-7-[(2-aminophenyl)amino]-2,3-dihydro-1-benzofuran-3-yl}(trifluoroacetyl)amino]-2,3-dihydro-1-benzofuran-3-yl}acetate (160 mg, 0.303 mmol) and tetraethoxymethane (190 μL, 0.909 mmol).

MS m/z 582 (M+H)⁺.

### Reference Example 25

### methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (5.00 g, 10.0 mmol), 5-fluoro-2-nitroaniline (1.87 g, 12.0 mmol) and tripotassium phosphate (4.25 g, 20.0 mmol) in toluene (50 mL) were added under an argon atmosphere tris(dibenzylideneacetone)dipalladium (0) (458 mg. 0.500 mmol) and dicyclohexyl[2′,4′,6′-tri(propan-2-yl)biphenyl-2-yl]phosphane (476 mg, 1.00 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give a red dark oil (6.63 g). The obtained oil (6.63 g) was dissolved in methanol (50 mL) and tetrahydrofuran (25 mL), 10% palladium-carbon (50% water-containing product, 1.15 g) was added, and the mixture was stirred under a hydrogen atmosphere (balloon pressure) at room temperature for 5 hr. The catalyst was filtered off, and the filtrate was concentrated under reduced pressure. The residue was purified by silica gel column chromatography (hexane:ethyl acetate=95:5-50:50) to give the title compound (4.94 g, yield 91%) as a purple solid.

MS m/z 546 (M+H)⁺.

### Reference Example 26

### methyl [(3S)-6-{[(3S)-7-(2-ethyl-6-fluoro-1H-benzimidazol-1-yl)-2,3-dihydro-1-benzofuran-3-yl](trifluoroacetyl)amino}-2,3-dihydro-1-benzofuran-3-yl]acetate

In the same manner as in Reference Example 16, the title compound (147 mg, yield 80%) was obtained as a white solid from methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate (160 mg, 0.293 mmol) and propanoyl chloride (30.7 μL, 0.352 mmol).

MS m/z 584 (M+H)⁺.

### Reference Example 27

### methyl [(3S)-6-{[(3S)-7-(2-ethoxy-6-fluoro-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate (3.00 g, 5.50 mmol) and tetraethoxymethane (3.45 mL, 16.5 mmol) in acetic acid (28 mL) was stirred at 80° C. for 2 hr. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-60:40), and triturated with hexane-ethyl acetate to give the title compound (2.49 g, yield 75%) as a pale-yellow solid.

MS m/z 600 (M+H)⁺.

### Reference Example 28

### methyl {(3S)-6-[{(3S)-7-[(4,5-difluoro-2-nitrophenyl)amino]-2,3-dihydro-1-benzofuran-3-yl}(trifluoroacetyl)amino]-2,3-dihydro-1-benzofuran-3-yl}acetate

In the same manner as in Reference Example 18, the title compound (370 mg, yield 100%) was obtained as an orange oil from methyl [(3S)-6-{[(3S)-7-bromo-2,3-dihydro-1-benzofuran-3-yl](trifluoroacetyl)amino}-2,3-dihydro-1-benzofuran-3-yl]acetate (300 mg, 0.600 mmol) and 4,5-difluoro-2-nitroaniline (115 mg, 0.660 mmol).

MS m/z 592 (M−H)⁻.

### Reference Example 29

### methyl {(3S)-6-[{(3S)-7-[(2-amino-4,5-difluorophenyl)amino]-2,3-dihydro-1-benzofuran-3-yl}(trifluoroacetyl)amino]-2,3-dihydro-1-benzofuran-3-yl}acetate

In the same manner as in Reference Example 19, the title compound (302 mg, yield 89%) was obtained as a purple oil from methyl {(3S)-6-[{(3S)-7-[(4,5-difluoro-2-nitrophenyl)amino]-2,3-dihydro-1-benzofuran-3-yl}(trifluoroacetyl)amino]-2,3-dihydro-1-benzofuran-3-yl}acetate (0.600 mmol).

MS m/z 564 (M+H)⁺.

### Reference Example 30

### methyl [(3S)-6-{[(3S)-7-(2-ethyl-5,6-difluoro-1H-benzimidazol-1-yl)-2,3-dihydro-1-benzofuran-3-yl](trifluoroacetyl)amino}-2,3-dihydro-1-benzofuran-3-yl]acetate

In the same manner as in Reference Example 16, the title compound (130 mg, yield 81%) was obtained as a colorless oil from methyl {(3S)-6-[{(3S)-7-[(2-amino-4,5-difluorophenyl)amino]-2,3-dihydro-1-benzofuran-3-yl}(trifluoroacetyl)amino]-2,3-dihydro-1-benzofuran-3-yl}acetate (151 mg, 0.267 mmol) and propanoyl chloride (28.0 μL, 0.321 mmol).

MS m/z 602 (M+H)⁺.

### Reference Example 31

### methyl [(3S)-6-{[(3S)-7-(2-ethoxy-5,6-difluoro-1H-benzimidazol-1-yl)-2,3-dihydro-1-benzofuran-3-yl](trifluoroacetyl)amino}-2,3-dihydro-1-benzofuran-3-yl]acetate

In the same manner as in Reference Example 17, the title compound (98.6 mg, yield 60%) was obtained as a pale-pink solid from methyl {(3S)-6-[{(3S)-7-[(2-amino-4,5-difluorophenyl)amino]-2,3-dihydro-1-benzofuran-3-yl}(trifluoroacetyl)amino]-2,3-dihydro-1-benzofuran-3-yl}acetate (151 mg, 0.267 mmol) and tetraethoxymethane (168 μL, 0.802 mmol).

MS m/z 618 (M+H)⁺.

### Reference Example 32

### methyl {(3S)-6-[{(3S)-7-[(2,3-difluoro-6-nitrophenyl)amino]-2,3-dihydro-1-benzofuran-3-yl}(trifluoroacetyl)amino]-2,3-dihydro-1-benzofuran-3-yl}acetate

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (5.00 g, 10.0 mmol), 2,3-difluoro-6-nitroaniline (2.09 g, 12.0 mmol) and tripotassium phosphate (4.25 g, 20.0 mmol) in toluene (50 mL) were added under an argon atmosphere tris(dibenzylideneacetone)dipalladium (0) (459 mg, 0-0.500 mmol) and dicyclohexyl[2′,4′,6′-tri(propan-2-yl)biphenyl-2-yl]phosphane (477 mg, 1.00 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give the title compound (5.41 g, yield 91%) as an orange oil.

MS m/z 592 (M−H)⁻.

### Reference Example 33

### methyl {(3S)-6-[{(3S)-7-[(6-amino-2,3-difluorophenyl)amino]-2,3-dihydro-1-benzofuran-3-yl}(trifluoroacetyl)amino]-2,3-dihydro-1-benzofuran-3-yl}acetate

Methyl {(3S)-6-[{(3S)-7-[(2,3-difluoro-6-nitrophenyl)amino]-2,3-dihydro-1-benzofuran-3-yl}(trifluoroacetyl)amino]-2,3-dihydro-1-benzofuran-3-yl}acetate (5.41 g, 9.12 mmol) was dissolved in methanol (50 mL) and tetrahydrofuran (25 mL), 10% palladium-carbon (50% water-containing product, 1.08 g) was added, and the mixture was stirred under a hydrogen atmosphere (balloon pressure) at room temperature for 4 hr. The catalyst was filtered off, and the filtrate was concentrated under reduced pressure. The residue was purified by silica gel column chromatography (hexane:ethyl acetate=95:5-20:80) to give the title compound (4.50 g, yield 88%) as a black solid.

MS m/z 564 (M+H)⁺.

### Reference Example 34

### methyl [(3S)-6-{[(3S)-7-(2-ethyl-6,7-difluoro-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3S)-7-[(6-amino-2,3-difluorophenyl)amino]-2,3-dihydro-1-benzofuran-3-yl}(trifluoroacetyl)amino]-2,3-dihydro-1-benzofuran-3-yl}acetate (3.50 g, 6.21 mmol) in N,N-dimethylacetamide (31 mL) was ice-cooled, and propanoyl chloride (0.651 mL, 7.45 mmol) was added dropwise. The reaction mixture was stirred at room temperature for 1 hr, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (20 mL) was stirred overnight at 130° C. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over anhydrous magnesium sulfate, and concentrated under reduced pressure. The residue was purified by basic silica gel column chromatography (hexane:ethyl acetate=95:5-40:60) to give the title compound (3.72 g, yield 100%) as a yellow oil.

MS m/z 602 (M+H)⁺.

### Reference Example 35

### methyl [(3S)-6-{[(3S)-7-(2-ethoxy-6,7-difluoro-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3S)-7-[(6-amino-2,3-difluorophenyl)amino]-2,3-dihydro-1-benzofuran-3-yl}(trifluoroacetyl)amino]-2,3-dihydro-1-benzofuran-3-yl}acetate (2.25 g, 4.00 mmol) and tetraethoxymethane (2.51 mL, 12.0 mmol) in acetic acid (20 mL) was stirred at 60° C. for 2 hr. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) and basic silica gel column chromatography (hexane:ethyl acetate=95:5-40:60) to give the title compound (1.87 g, yield 76%) as a yellow oil.

MS m/z 618 (M+H)⁺.

### Reference Example 36

### methyl {(3S)-6-[{(3S)-7-[(3,5-difluoro-2-nitrophenyl)amino]-2,3-dihydro-1-benzofuran-3-yl}(trifluoroacetyl)amino]-2,3-dihydro-1-benzofuran-3-yl}acetate

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (5.00 g, 10.0 mmol) and 3,5-difluoro-2-nitroaniline (2.09 g, 12.0 mmol) and tripotassium phosphate (4.25 g, 20.0 mmol) in toluene (50 mL) were added under an argon atmosphere tris(dibenzylideneacetone)dipalladium (0) (458 mg, 0.500 mmol) and dicyclohexyl[2′,4′,6′-tri(propan-2-yl)biphenyl-2-yl]phosphane (477 mg, 1.00 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-60:40) to give the title compound (6.64 g, yield 100%) as a red orange oil.

MS m/z 592 (M−H)⁻.

### Reference Example 37

### methyl {(3S)-6-[{(3S)-7-[(2-amino-3,5-difluorophenyl)amino]-2,3-dihydro-1-benzofuran-3-yl}(trifluoroacetyl)amino]-2,3-dihydro-1-benzofuran-3-yl}acetate

Methyl {(3S)-6-[{(3S)-7-[(3,5-difluoro-2-nitrophenyl)amino]-2,3-dihydro-1-benzofuran-3-yl}(trifluoroacetyl)amino]-2,3-dihydro-1-benzofuran-3-yl}acetate (10.0 mmol) was dissolved in methanol (50 mL) and tetrahydrofuran (25 mL), 10% palladium-carbon (50% water-containing product, 1.33 g) was added, and the mixture was stirred under a hydrogen atmosphere (balloon pressure) at room temperature for 4 hr. The catalyst was filtered off, and the filtrate was concentrated under reduced pressure. The residue was purified by silica gel column chromatography (hexane:ethyl acetate=95:5-60:40) and triturated with hexane-toluene to give the title compound (4.49 g, yield 80%) as a pale-orange solid.

MS m/z 564 (M+H)⁺.

### Reference Example 38

### methyl [(3S)-6-{[(3S)-7-(2-ethyl-4,6-difluoro-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3S)-7-[(2-amino-3,5-difluorophenyl)amino]-2,3-dihydro-1-benzofuran-3-yl}(trifluoroacetyl)amino]-2,3-dihydro-1-benzofuran-3-yl}acetate (122 mg, 0.216 mmol) in N,N-dimethylacetamide (1 mL) was ice-cooled, and propanoyl chloride (22.6 μL, 0.259 mmol) was added dropwise. The reaction mixture was stirred at room temperature for 30 min, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (1 mL) was stirred overnight at 140° C. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-50:50) to give the title compound (106 mg, yield 82%) as a colorless oil.

MS m/z 602 (M+H)⁺.

### Reference Example 39

### methyl {(3S)-6-[{(3S)-7-[6-fluoro-2-(tetrahydro-2H-pyran-4-yl)-1H-benzimidazol-1-yl]-2,3-dihydro-1-benzofuran-3-yl}(trifluoroacetyl)amino]-2,3-dihydro-1-benzofuran-3-yl}acetate

To a solution of methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate (150 mg, 0.275 mmol) and tetrahydro-2H-pyran-4-carboxylic acid (37.6 mg, 0.289 mmol) in tetrahydrofuran (1.4 mL) were added N-(3-dimethylaminopropyl)-N′-ethylcarbodiimide hydrochloride (63.3 mg, 0.330 mmol) and 1-hydroxybenzotriazole monohydrate (50.5 mg, 0.330 mmol). The reaction mixture was stirred overnight at room temperature, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (1.4 mL) was stirred overnight at 130° C. The reaction mixture was cooled to room temperature, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-30:70) to give the title compound (106 mg, yield 60%) as a pale-yellow solid.

MS m/z 640 (M+H)⁺.

### Reference Example 40

### methyl [(3S)-6-{[(3S)-7-(2-ethoxy-4,6-difluoro-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3S)-7-[(2-amino-3,5-difluorophenyl)amino]-2,3-dihydro-1-benzofuran-3-yl}(trifluoroacetyl)amino]-2,3-dihydro-1-benzofuran-3-yl}acetate (3.00 g, 5.32 mmol) and tetraethoxymethane (3.35 mL, 16.0 mmol) in acetic acid (27 mL) was stirred at 60° C. for 3 hr. The reaction mixture was cooled to room temperature, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) and triturated with hexane to give the title compound (2.04 g, yield 62%) as a pale-orange solid.

MS m/z 618 (M+H)⁺.

### Reference Example 41

### methyl {(3S)-6-[{(3S)-7-[(5-methoxy-2-nitrophenyl)amino]-2,3-dihydro-1-benzofuran-3-yl}(trifluoroacetyl)amino]-2,3-dihydro-1-benzofuran-3-yl}acetate

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (150 mg, 0.300 mmol) and 5-methoxy-2-nitroaniline (55.5 mg, 0.330 mmol) and tripotassium phosphate (127 mg, 0.600 mmol) in toluene (1.5 mL) were added under an argon atmosphere tris(dibenzylideneacetone)dipalladium (0) (13.7 mg, 0.015 mmol) and dicyclohexyl[2′,4′,6′-tri(propan-2-yl)biphenyl-2-yl]phosphane (14.3 mg, 0.030 mmol), and the mixture was stirred at 100° C. overnight. The reaction mixture was cooled to room temperature, filtered through celite, and concentrated under reduced pressure. The residue was purified by silica gel column chromatography (hexane:ethyl acetate=95:5-60:40) to give the title compound (181 mg, yield 100%) as a yellow oil.

MS m/z 588 (M+H)⁺.

### Reference Example 42

### methyl {(3S)-6-[{(3S)-7-[(2-amino-5-methoxyphenyl)amino]-2,3-dihydro-1-benzofuran-3-yl}(trifluoroacetyl)amino]-2,3-dihydro-1-benzofuran-3-yl}acetate

Methyl {(3S)-6-[{(3S)-7-[(5-methoxy-2-nitrophenyl)amino]-2,3-dihydro-1-benzofuran-3-yl}(trifluoroacetyl)amino]-2,3-dihydro-1-benzofuran-3-yl}acetate (0.300 mmol) was dissolved in methanol (1.5 mL) and tetrahydrofuran (0.8 mL), 10% palladium-carbon (50% water-containing product, 36.0 mg) was added, and the mixture was stirred under a hydrogen atmosphere (balloon pressure) at room temperature for 3 hr. The catalyst was filtered off, and the filtrate was concentrated under reduced pressure. The residue was purified by silica gel column chromatography (hexane:ethyl acetate=95:5-50:50) to give the title compound (133 mg, yield 79%) as a pale-brown oil.

MS m/z 558 (M+H)⁺.

### Reference Example 43

### methyl [(3S)-6-{[(3S)-7-(2-ethyl-6-methoxy-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{(3S)-7-[(2-amino-5-methoxyphenyl)amino]-2,3-dihydro-1-benzofuran-3-yl}(trifluoroacetyl)amino]-2,3-dihydro-1-benzofuran-3-yl}acetate (133 mg, 0.238 mmol) in N,N-dimethylacetamide (1.2 mL) was ice-cooled, and propanoyl chloride (25.0 μL, 0.286 mmol) was added dropwise. The reaction mixture was stirred at room temperature for 30 min, saturated aqueous sodium hydrogen carbonate was added, and the mixture was extracted with ethyl acetate. The extract was washed with saturated brine, dried over anhydrous magnesium sulfate, and concentrated under reduced pressure. To the obtained residue was added acetic acid (1 mL), and the mixture was stirred at 140° C. overnight. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) to give the title compound (101 mg, yield 71%) as a colorless oil.

MS m/z 596 (M+H)⁺.

### Reference Example 44

### methyl {(3S)-6-[{(3S)-7-[6-fluoro-2-(propan-2-yl)-1H-benzimidazol-1-yl]-2,3-dihydro-1-benzofuran-3-yl}(trifluoroacetyl)amino]-2,3-dihydro-1-benzofuran-3-yl}acetate

A solution of methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate (160 mg, 0.293 mmol) in N,N-dimethylacetamide (1.5 mL) was ice-cooled, and 2-methylpropanoyl chloride (37.5 mg, 0.352 mmol) was added dropwise. The reaction mixture was stirred at room temperature for 20 min, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (1 mL) was stirred overnight at 140° C. The reaction mixture was cooled to room temperature, concentrated under reduced pressure to remove acetic acid, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-50:50) to give the title compound (134 mg, yield 76%) as a pale-yellow oil.

MS m/z 598 (M+H)⁺.

### Reference Example 45

### methyl {(3S)-6-[{(3S)-7-[6-fluoro-2-(tetrahydro-2H-pyran-4-yl)-1H-benzimidazol-1-yl]-2,3-dihydro-1-benzofuran-3-yl}(trifluoroacetyl)amino]-2,3-dihydro-1-benzofuran-3-yl}acetate

To a solution of methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate (150 mg, 0.275 mmol) and tetrahydro-2H-pyran-4-carboxylic acid (37.6 mg, 0.289 mmol) in tetrahydrofuran (1.4 mL) were added N-(3-dimethylaminopropyl)-N′-ethylcarbodiimide hydrochloride (63.3 mg, 0.330 mmol) and 1-hydroxybenzotriazole monohydrate (50.5 mg, 0.330 mmol). The reaction mixture was stirred overnight at room temperature, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (1.4 mL) was stirred overnight at 130° C. The reaction mixture was cooled to room temperature, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-30:70) to give the title compound (106 mg, yield 60%) as a pale-yellow solid.

MS m/z 640 (M+H)⁺.

### Reference Example 46

### methyl {(3S)-6-[{(3S)-7-[6-fluoro-2-(5-methylfuran-2-yl)-1H-benzimidazol-1-yl]-2,3-dihydro-1-benzofuran-3-yl}(trifluoroacetyl)amino]-2,3-dihydro-1-benzofuran-3-yl}acetate

To a solution of methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate (150 mg, 0.275 mmol) and 5-methylfuran-2-carboxylic acid (36.4 mg, 0.289 mmol) in tetrahydrofuran (1.4 mL) were added N-(3-dimethylaminopropyl)-N′-ethylcarbodiimide hydrochloride (63.3 mg, 0.330 mmol) and 1-hydroxybenzotriazole monohydrate (50.5 mg, 0.330 mmol). The reaction mixture was stirred overnight at room temperature, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (1.4 mL) was stirred overnight at 130° C. The reaction mixture was cooled to room temperature, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) to give the title compound (116 mg, yield 66%) as a pale-yellow oil.

MS m/z 636 (M+H)⁺.

### Reference Example 47

### methyl {(3S)-6-[{(3S)-7-[6-fluoro-2-(5-methylisoxazol-3-yl)-1H-benzimidazol-1-yl]-2,3-dihydro-1-benzofuran-3-yl}(trifluoroacetyl)amino]-2,3-dihydro-1-benzofuran-3-yl}acetate

A solution of methyl {(3S)-6-[{(3S)-7-[(2-amino-5-fluorophenyl)amino]-2,3-dihydro-1-benzofuran-3-yl}(trifluoroacetyl)amino]-2,3-dihydro-1-benzofuran-3-yl}acetate (150 mg, 0.275 mmol) in N,N-dimethylacetamide (1.4 mL) was ice-cooled, and 5-methylisoxazole-3-carbonyl chloride (48.0 mg, 0.330 mmol) was added dropwise. The reaction mixture was stirred at 0° C. for 20 min, saturated aqueous sodium hydrogen carbonate was added, and the mixture was extracted with ethyl acetate. The extract was washed with saturated brine, dried over anhydrous magnesium sulfate, and concentrated under reduced pressure. A solution of the obtained residue in acetic acid (1 mL) was stirred overnight at 120° C. The reaction mixture was cooled to room temperature, neutralized with saturated aqueous sodium hydrogen carbonate, and extracted with ethyl acetate. The extract was washed with saturated brine, dried over anhydrous magnesium sulfate, and concentrated under reduced pressure. The residue was purified by silica gel column chromatography (hexane:ethyl acetate=95:5-50:50) to give the title compound (104 mg, yield 60%) as a white solid.

MS m/z 637 (M+H)⁺.

### Reference Example 48

### methyl {(3S)-6-[{7-[(4-methyl-2-nitrophenyl)amino]-2,3-dihydro-1-benzofuran-3-yl}(trifluoroacetyl)amino]-2,3-dihydro-1-benzofuran-3-yl}acetate

To a mixture of methyl {(3S)-6-[(7-bromo-2,3-dihydro-1-benzofuran-3-yl)(trifluoroacetyl)amino]-2,3-dihydro-1-benzofuran-3-yl}acetate (1.63 g, 3.26 mmol), 4-methyl-2-nitroaniline (0.545 g, 3.58 mmol) and tripotassium phosphate (1.38 g, 6.52 mmol) in toluene (15 ml) were added tris(dibenzylideneacetone)dipalladium (0) (0.149 g, 0.163 mmol) and 2-dicyclohexylphosphino-2′,4′,6′-triisopropylbiphenyl (0.155 g, 0.326 mmol), and the mixture was stirred under an argon atmosphere at 100° C. for 13 hr. To the reaction mixture were added water and ethyl acetate, and the insoluble material was filtered off through celite. The organic layer of the filtrate was washed with saturated brine, dried over anhydrous magnesium sulfate, and concentrated under reduced pressure. The residue was purified by silica gel column chromatography (ethyl acetate:hexane=5:95-40:60) to give the title compound (1.68 g, yield 90%) as a red-brown viscous oil.

MS m/z 572 (M+H)⁺.

### Reference Example 49

### methyl {(3S)-6-[{7-[(2-amino-4-methylphenyl)amino]-2,3-dihydro-1-benzofuran-3-yl}(trifluoroacetyl)amino]-2,3-dihydro-1-benzofuran-3-yl}acetate

Methyl {(3S)-6-[{7-[(4-methyl-2-nitrophenyl)amino]-2,3-dihydro-1-benzofuran-3-yl}(trifluoroacetyl)amino]-2,3-dihydro-1-benzofuran-3-yl}acetate (1.68 g, 2.94 mmol) was dissolved in a mixed solution of methanol (10 mL) and tetrahydrofuran (5 mL), 10% palladium-carbon (50% water-containing product, 0.3 g) was added, and the mixture was stirred under a hydrogen atmosphere (balloon pressure) at room temperature for 21 hr. The catalyst was filtered off, and the filtrate was concentrated under reduced pressure. The residue was purified by silica gel column chromatography (ethyl acetate:hexane=20:80-70:30) to give the title compound (1.38 g, yield 87%) as a yellow viscous oil.

MS m/z 542 (M+H)⁺.

### Reference Example 50

### methyl [(3S)-6-{[7-(2,5-dimethyl-1H-benzimidazol-1-yl)-2,3-dihydro-1-benzofuran-3-yl](trifluoroacetyl)amino}-2,3-dihydro-1-benzofuran-3-yl]acetate

A solution of methyl (3S)-6-[{7-[(2-amino-4-methylphenyl)amino]-2,3-dihydro-1-benzofuran-3-yl}(trifluoroacetyl)amino]-2,3-dihydro-1-benzofuran-3-yl}acetate (1.38 g, 2.55 mmol) and acetic anhydride (2 mL, 21.2 mmol) in acetic acid (8 mL) was heated under reflux for 24 hr. The reaction mixture was concentrated under reduced pressure, the residue was diluted with ethyl acetate and washed with saturated aqueous sodium hydrogen carbonate and then saturated brine. The organic layer was dried over anhydrous magnesium sulfate, and concentrated under reduced pressure. The residue was purified by silica gel column chromatography (ethyl acetate:hexane=40:60-100:0), then preparative HPLC to give the title compound (1.09 g, yield 76%) as a pale-yellow foam.

MS m/z 566 (M+H)⁺.

### Reference Example 51

### methyl {(3S)-6-[{7-[(5-fluoro-2-nitrophenyl)amino]-2,3-dihydro-1-benzofuran-3-yl}(trifluoroacetyl)amino]-2,3-dihydro-1-benzofuran-3-yl}acetate

In the same manner as in Reference Example 48, the title compound was obtained as a brown viscous oil from methyl {(3S)-6-[(7-bromo-2,3-dihydro-1-benzofuran-3-yl)(trifluoroacetyl)amino]-2,3-dihydro-1-benzofuran-3-yl}acetate and 5-fluoro-2-nitroaniline. yield 97%.

MS (ESI−) m/z 574 (M−H)⁻.

### Reference Example 52

### methyl {(3S)-6-[{7-[(2-amino-5-fluorophenyl)amino]-2,3-dihydro-1-benzofuran-3-yl}(trifluoroacetyl)amino]-2,3-dihydro-1-benzofuran-3-yl}acetate

Methyl {(3S)-6-[{7-[(5-fluoro-2-nitrophenyl)amino]-2,3-dihydro-1-benzofuran-3-yl}(trifluoroacetyl)amino]-2,3-dihydro-1-benzofuran-3-yl}acetate (0.676 g, 1.175 mmol) was dissolved in a mixed solution of methanol (4 mL) and tetrahydrofuran (2 mL), 10% palladium-carbon (50% water-containing product, 0.2 g) was added, and the mixture was stirred under a hydrogen atmosphere (balloon pressure) at room temperature for 22 hr. The catalyst was filtered off, and the filtrate was concentrated under reduced pressure. The residue was purified by silica gel column chromatography (ethyl acetate:hexane=20:80-70:30) to give the title compound (0.483 g, yield 75%) as a purple viscous oil.

MS m/z 546 (M+H)⁺.

### Reference Example 53

### methyl [(3S)-6-{[7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate

A solution of methyl {(3S)-6-[{7-[(2-amino-5-fluorophenyl)amino]-2,3-dihydro-1-benzofuran-3-yl}(trifluoroacetyl)amino]-2,3-dihydro-1-benzofuran-3-yl}acetate (0.478 g, 0.876 mmol) and acetic anhydride (1 mL, 10.6 mmol) in acetic acid (4 mL) was heated under reflux for 24 hr. The reaction mixture was concentrated under reduced pressure, the residue was diluted with ethyl acetate, and the mixture was washed with saturated aqueous sodium hydrogen carbonate, then saturated brine. The organic layer was dried over anhydrous magnesium sulfate, and concentrated under reduced pressure. The residue was purified by silica gel column chromatography (ethyl acetate:hexane=40:60-100:0), then preparative HPLC to give the title compound (0.434 g, yield 87%) as a yellow viscous oil.

MS m/z 570 (M+H)⁺.

### Reference Example 54

### methyl [(3S)-6-{[(3S)-7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate and methyl [(3S)-6-{[(3R)-7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate

Methyl [(3S)-6-{[7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate was purified under the following conditions of high performance liquid chromatography to give methyl [(3S)-6-{[(3S)-7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate (0.309 g, yield 43%) with an enantiometric excess of 99.9% and methyl [(3S)-6-{[(3R)-7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate (0.317 g, yield 44%) with an enantiometric excess of 99.8%.

(high performance liquid chromatography conditions)
column: CHIRALCEL OJ (manufactured by Daicel Chemical Industries, Ltd.)
mobile phase: n-hexane/ethanol (volume ratio: 20/80)
flow rate: 30 mL/min
detection: UV (220 nm)
temperature: 30° C.

### Reference Example 55

### methyl {(3S)-6-[{(3S)-7-[(5-fluoropyridin-2-yl)amino]-2,3-dihydro-1-benzofuran-3-yl}(trifluoroacetyl)amino]-2,3-dihydro-1-benzofuran-3-yl}acetate

A solution of methyl [(3S)-6-{[(3S)-7-bromo-2,3-dihydro-1-benzofuran-3-yl](trifluoroacetyl)amino}-2,3-dihydro-1-benzofuran-3-yl]acetate (0.200 g, 0.400 mmol), 5-fluoropyridin-2-amine (53.8 mg, 0.480 mmol) and cesium carbonate (0.261 g, 0.800 mmol) in toluene (4 mL) was substituted with argon, and tris(dibenzylideneacetone)dipalladium (0) (14.6 mg, 0.0160 mmol) and 4,5-bis(diphenylphosphino)-9,9-dimethylxanthene (18.5 mg, 0.0320 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-65:35) to give the title compound (0.186 mg, yield 88%) as a brown oil.

MS m/z 532 (M+H)⁺.

### Reference Example 56

### 4-(4,6-dimethylpyrimidin-2-yl)morpholine

A mixed solution of 2-chloro-4,6-dimethylpyrimidine (3.00 g, 21.0 mmol) and morpholine (15 mL) was stirred using microwave (Biotage Initiator™60) at 210° C. for 10 min. The reaction mixture was diluted with water, and the mixture was extracted with ethyl acetate. The extract was washed with water, dried over sodium sulfate, and concentrated under reduced pressure to give the title compound (3.74 g, yield 92%) as a pale-yellow solid.

¹H NMR (300 MHz, CDCl₃) δ 2.29 (6H, s), 3.67-3.87 (8H, m), 6.30 (1H, s).

### Reference Example 57

### 4-(5-bromo-4,6-dimethylpyrimidin-2-yl)morpholine

To a solution of 4-(4,6-dimethylpyrimidin-2-yl)morpholine (10.7 g, 55.6 mmol) in ethyl acetate (80 mL) was added N-bromosuccinimide (10.1 g, 55.6 mmol) at room temperature, and the mixture was heated under reflux for 5 min. The reaction mixture was diluted with 1 M aqueous sodium hydroxide solution, and the mixture was extracted with ethyl acetate. The extract was washed with water, dried over sodium sulfate, and concentrated under reduced pressure to give the title compound (14.7 g, yield 0.97%) as a pale-yellow solid.

¹H NMR (300 MHz, CDCl₃) δ 2.45 (6H, s), 3.69-3.81 (8H, m).

### Reference Example 58

### (4,6-dimethyl-2-morpholin-4-ylpyrimidin-5-yl)boronic acid

To a solution of 4-(5-bromo-4,6-dimethylpyrimidin-2-yl)morpholine (14.7 g, 54.0 mmol) in tetrahydrofuran (140 mL) was added dropwise at −78° C. a hexane solution (1.6 M, 35.4 mL, 56.7 mmol) of n-butyllithium. The reaction mixture was stirred at the same temperature for 40 min, triisopropyl borate (24.8 mL, 108 mmol) was added, and the mixture was heated to room temperature and stirred for 5 hr. The reaction mixture was poured into aqueous ammonium chloride solution, the mixture was stirred for 4 hr and extracted with ethyl acetate. The extract was washed with saturated brine, dried over anhydrous sodium sulfate, and concentrated under reduced pressure. To the residue was added diisopropyl ether, and the resulting precipitate was collected by filtration, and dried to give the title compound (7.42 g, yield 58%) as a pale-yellow solid.

¹H NMR (300 MHz, DMSO-d₆) δ 2.25 (6H, s), 3.52-3.73 (8H, m), 8.19 (2H, s).

### Reference Example 59

### methyl [(3S)-6-{[(3S)-7-(4,6-dimethyl-2-morpholin-4-ylpyrimidin-5-yl)-2,3-dihydro-1-benzofuran-3-yl](trifluoroacetyl)amino}-2,3-dihydro-1-benzofuran-3-yl]acetate

Methyl [(3S)-6-{[(3S)-7-bromo-2,3-dihydro-1-benzofuran-3-yl](trifluoroacetyl)amino}-2,3-dihydro-1-benzofuran-3-yl]acetate (0.450 g, 0.900 mmol) and (4,6-dimethyl-2-morpholin-4-ylpyrimidin-5-yl)boronic acid (0.256 g, 1.08 mmol) were suspended in a mixed solution of 2 M aqueous sodium carbonate solution (1.08 mL) and toluene (6 mL) and, after argon substitution, tris(dibenzylideneacetone)dipalladium (0) (32.9 mg, 0.0360 mmol) and 2-dicyclohexylphosphino-2′,6′-dimethoxybiphenyl (60.9 mg, 0.144 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-60:40) to give the title compound (0.551 g, yield 100%) as a pale-yellow oil.

MS m/z 613 (M+H)⁺.

### Reference Example 60

### 4,6-dimethyl-2-pyrrolidin-1-ylpyrimidine

A solution of 2-chloro-4,6-dimethylpyrimidine (2.00 g, 14.0 mmol), pyrrolidine (4 mL) and triethylamine (1.96 mL, 14.0 mmol) in ethanol (10 mL) was stirred by microwave at 150° C. for 10 min. The reaction mixture was diluted with water, and the mixture was extracted with ethyl acetate. The extract was washed with water, dried over sodium sulfate, and concentrated under reduced pressure to give the title compound (2.20 g, yield 88%) as a pale-yellow solid.

¹H NMR (300 MHz, CDCl₃) δ 1.87-2.01 (4H, m), 2.29 (6H, s), 3.51-3.65 (4H, m), 6.23 (1H, s).

MS m/z 178 (M+H)⁺.

### Reference Example 61

### 5-bromo-4,6-dimethyl-2-pyrrolidin-1-ylpyrimidine

To a solution of 4,6-dimethyl-2-pyrrolidin-1-ylpyrimidine (2.20 g, 12.4 mmol) in chloroform (10 mL) was added N-bromosuccinimide (2.25 g, 12.4 mmol) at room temperature and the mixture was heated under reflux for 5 min. The reaction mixture was diluted with 1 M sodium hydroxide, and extracted with ethyl acetate. The extract was washed with water, dried over sodium sulfate, and concentrated under reduced pressure. The residue was purified by silica gel column chromatography (hexane:ethyl acetate=100:0-75:25) to give the title compound (3.00 g, yield 94%) as a white solid.

¹H NMR (300 MHz, CDCl₃) δ 1.88-2.01 (4H, m), 2.45 (6H, s), 3.46-3.58 (4H, m).

MS m/z 256 (M+H)⁺.

### Reference Example 62

### (4,6-dimethyl-2-pyrrolidin-1-ylpyrimidin-5-yl)boronic acid

To a solution of 5-bromo-4,6-dimethyl-2-pyrrolidin-1-ylpyrimidine (1.52 g, 5.93 mmol) in tetrahydrofuran (20 mL) was added dropwise at −78° C. a hexane solution (1.6 M, 3.89 mL, 6.23 mmol) of n-butyllithium. The reaction mixture was stirred at the same temperature for 40 min, triisopropyl borate (2.72 mL, 11.9 mmol) was added, and the mixture was heated to room temperature and stirred for 5 hr. The reaction mixture was poured into aqueous ammonium chloride solution, and the mixture was stirred for 4 hr and extracted with ethyl acetate. The extract was washed with saturated brine, dried over anhydrous magnesium sulfate, and concentrated under reduced pressure. The obtained solid was washed with hexane and diisopropyl ether to give the title compound (0.766 g, yield 58%) as a white solid.

MS m/z 222 (M+H)⁺.

### Reference Example 63

### methyl [(3S)-6-{[(3S)-7-(4,6-dimethyl-2-pyrrolidin-1-ylpyrimidin-5-yl)-2,3-dihydro-1-benzofuran-3-yl](trifluoroacetyl)amino}-2,3-dihydro-1-benzofuran-3-yl]acetate

In the same manner as in Reference Example 59, the title compound was obtained as a pale-yellow non-crystalline powder from methyl [(3S)-6-{[(3S)-7-bromo-2,3-dihydro-1-benzofuran-3-yl](trifluoroacetyl)amino}-2,3-dihydro-1-benzofuran-3-yl]acetate and (4,6-dimethyl-2-pyrrolidin-1-ylpyrimidin-5-yl)boronic acid. yield 96%.

MS m/z 597 (M+H)⁺.

### Reference Example 64

### methyl [(3S)-6-{[(3S)-7-anilino-2,3-dihydro-1-benzofuran-3-yl](trifluoroacetyl)amino}-2,3-dihydro-1-benzofuran-3-yl]acetate

A solution of methyl [(3S)-6-{[(3S)-7-bromo-2,3-dihydro-1-benzofuran-3-yl](trifluoroacetyl)amino}-2,3-dihydro-1-benzofuran-3-yl]acetate (0.170 g, 0.339 mmol), aniline (57.0 mL, 0.0270 mmol) and cesium carbonate (0.221 g, 0.678 mmol) in toluene (4 mL) was substituted with argon, and tris(dibenzylideneacetone)dipalladium (0) (12.4 mg, 0.0140 mmol) and 4,5-bis(diphenylphosphino)-9,9-dimethylxanthene (15.7 mg, 0.0270 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-70:30) to give the title compound (0.167 mg, yield 96%) as a yellow solid.

MS m/z 513 (M+H)⁺.

### Reference Example 65

### methyl {(3S)-6-[{(3S)-7-[ethyl(phenyl)amino]-2,3-dihydro-1-benzofuran-3-yl}(trifluoroacetyl)amino]-2,3-dihydro-1-benzofuran-3-yl}acetate

To a solution of methyl [(3S)-6-{[(3S)-7-anilino-2,3-dihydro-1-benzofuran-3-yl](trifluoroacetyl)amino}-2,3-dihydro-1-benzofuran-3-yl]acetate (0.167 g, 0.325 mmol) and ethyl iodide (51.0 μg, 0.488 mmol) in N,N-dimethylformamide (2 mL) was added sodium hydride (60% in oil, 15.6 mg, 0.390 mmol) at 0° C., and the mixture was stirred at room temperature for 1.5 hr. The reaction mixture was poured into water, and the mixture was extracted with ethyl acetate. The extract was dried over anhydrous sodium sulfate, and concentrated under reduced pressure. The residue was purified by silica gel column chromatography (hexane:ethyl acetate=100:0-75:25) to give the title compound (71.9 mg, yield 41%) as a pale-yellow oil.

MS m/z 541 (M+H)⁺.

### Reference Example 66

### methyl {(3S)-6-[{(3S)-7-[(4-methylpyridin-2-yl)amino]-2,3-dihydro-1-benzofuran-3-yl}(trifluoroacetyl)amino]-2,3-dihydro-1-benzofuran-3-yl}acetate

A solution of methyl [(3S)-6-{[(3S)-7-bromo-2,3-dihydro-1-benzofuran-3-yl](trifluoroacetyl)amino}-2,3-dihydro-1-benzofuran-3-yl]acetate (7.00 g, 14.0 mmol), 4-methylpyridin-2-amine (1.82 g, 16.8 mmol) and cesium carbonate (9.12 g, 28.0 mmol) in toluene (60 mL) was substituted with argon, and tris(dibenzylideneacetone)dipalladium (0) (0.513 g, 0.560 mmol) and 4,5-bis(diphenylphosphino)-9,9-dimethylxanthene (0.648 g, 1.12 mmol) were added. The reaction mixture was stirred under an argon atmosphere at 100° C. for 16 hr. The reaction mixture was cooled, insoluble material was filtered off, and washed with toluene. The filtrate was concentrated under reduced pressure and the obtained residue was purified by silica gel column chromatography (hexane:ethyl acetate=95:5-60:40) to give the title compound (1.98 g, yield 27%) as a yellow oil.

MS m/z 528 (M+H)⁺.

### Reference Example 67

### methyl [(3S)-6-{[(3S)-7-(2,3-dihydro-4H-1,4-benzooxazin-4-yl)-2,3-dihydro-1-benzofuran-3-yl](trifluoroacetyl)amino}-2,3-dihydro-1-benzofuran-3-yl]acetate

A solution of methyl [(3S)-6-{[(3S)-7-bromo-2,3-dihydro-1-benzofuran-3-yl](trifluoroacetyl)amino}-2,3-dihydro-1-benzofuran-3-yl]acetate (200 mg, 0.400 mmol), 3,4-dihydro-2H-1,4-benzooxazine (64.8 mg, 0.480 mmol) and cesium carbonate (390 mg, 1.20 mmol) in toluene (4 mL) was substituted with argon, and tris(dibenzylideneacetone)dipalladium (0) (14.6 mg, 0.0160 mmol) and 4,5-bis(diphenylphosphino)-9,9-dimethylxanthene (18.5 mg, 0.0320 mmol) were added. The reaction mixture was stirred under an argon atmosphere at 100° C. for 16 hr. The reaction mixture was cooled, and the insoluble material was filtered off, and washed with toluene. The filtrate was concentrated under reduced pressure and the obtained residue was purified by silica gel column chromatography (hexane:ethyl acetate=95:5-60:40) to give the title compound (181 mg, yield 82%) as a yellow oil.

MS m/z 555 (M+H)⁺.

### Reference Example 68

### 7-bromo-2,3-dihydro-1-benzofuran-3-amine

A mixed solution of 7-bromo-1-benzofuran-3(2H)-one (0.521 g, 2.45 mmol), O-methylhydroxyammonium chloride (0.306 g, 3.67 mmol) and sodium acetate (0.301 g, 3.67 mmol) in methanol (12 mL) was heated under reflux for 10 hr, and the mixture was stirred at room temperature for 16 hr. The reaction mixture was concentrated under reduced pressure, the residue was poured into saturated aqueous sodium hydrogen carbonate, and the mixture was extracted with ethyl acetate. The extract was washed with saturated brine, dried over sodium sulfate, and concentrated under reduced pressure. The residue was purified by silica gel column chromatography (hexane:ethyl acetate=100:0-80:20) to give 7-bromo-1-benzofuran-3(2H)-one O-methyloxime (0.468 g, yield 79%) as a yellow solid. To a solution of 7-bromo-1-benzofuran-3(2H)-one O-methyloxime (0.468 g, 1.93 mmol) obtained above in tetrahydrofuran (9 mL) was slowly added dropwise borane-tetrahydrofuran solution (1 M, 5.80 mL, 5.80 mmol) at room temperature, and the mixture was heated under reflux under a nitrogen atmosphere for 3 hr. The reaction mixture was cooled, ice water was slowly added, 1 M hydrochloric acid was added, and the mixture was stirred at 80° C. for 1.5 hr. The reaction mixture was allowed to cool, 28% aqueous ammonia solution was added to alkalify the solution, and the mixture was extracted with ethyl acetate. The extract was dried over sodium sulfate, and concentrated under reduced pressure. The residue (0.402 g) was dissolved in diethyl ether (4 mL), and 4 M hydrochloric acid-ethyl acetate solution (0.480 mL) was slowly added. The precipitated solid was collected by filtration, and washed with diethyl ether to give 7-bromo-2,3-dihydro-1-benzofuran-3-amine hydrochloride (0.434 g) as a beige solid. The solid obtained above was dissolved in 28% aqueous ammonia solution, and the mixture was extracted with ethyl acetate. The extract was washed with saturated brine, dried over sodium sulfate, and concentrated under reduced pressure to give the title compound (0.366 g, yield 89%) as a yellow solid.

¹H NMR (300 MHz, DMSO-d₆) δ 2.15 (2H, br s), 4.05-4.16 (1H, m), 4.58-4.72 (2H, m), 6.78-6.87 (1H, m), 7.28-7.38 (2H, m).

### Reference Example 69

### (3S)-7-(4,6-dimethyl-2-morpholin-4-ylpyrimidin-5-yl)-2,3-dihydro-1-benzofuran-3-amine

(2R)-{[(3,5-Dinitrophenyl)carbonyl]amino}(phenyl)ethanoic acid (0.181 g, 0.524 mmol) was dissolved in acetonitrile (38.5 mL), and a solution of 7-bromo-2,3-dihydro-1-benzofuran-3-amine (0.112 g, 0.524 mmol) in methanol (10 mL) was added. The mixed solution was stood at room temperature for 1 hr, and further stood in a refrigerator for 1 hr. The precipitated salt was collected by filtration, dried under reduced pressure at 40° C. for 1 hr to give (3S)-7-bromo-2,3-dihydro-1-benzofuran-3-aminium (2R)-{[(3,5-dinitrophenyl)carbonyl]amino}(phenyl)ethanoate (0.120 g, recovery rate 41%, enantiometric excess 96.3% d.e.) as a white solid.

(analysis conditions)
column: CHIRALCEL OD-RH (DF005) 4.6 mm ID×150 mm L
(manufactured by Daicel Chemical Industries, Ltd.)
mobile phase: 50 mM KPF₆ (pH 2, TFA)/acetonitrile (volume ratio: 850/150)
flow rate: 1 mL/min
detection: UV (220 nm)
concentration: 0.25 mg/mL (water/acetonitrile=85/15)
temperature: 300° C.
injection volume: 10 μL

To a solution of (3S)-7-bromo-2,3-dihydro-1-benzofuran-3-aminium (2R)-{[(3,5-dinitrophenyl)carbonyl]amino}(phenyl)ethanoate (0.117 g, 0.208 mmol) obtained above and triethylamine (0.290 mL, 2.08 mmol) in tetrahydrofuran (6 mL) was added di-tert-butyl bicarbonate (0.0580 mL, 0.250 mmol), and the mixture was stirred at room temperature for 3 hr. To the reaction mixture was added saturated aqueous sodium hydrogen carbonate, and the mixture was extracted with ethyl acetate. The extract was washed with 0.1 M hydrochloric acid solution, then saturated aqueous sodium hydrogen carbonate, dried over sodium sulfate, and concentrated under reduced pressure to give a crude product (0.102 g) of tert-butyl [(3S)-7-bromo-2,3-dihydro-1-benzofuran-3-yl]carbamate as a white solid. The obtained crude product (0.102 g) of tert-butyl [(3S)-7-bromo-2,3-dihydro-1-benzofuran-3-yl]carbamate and (4,6-dimethyl-2-morpholin-4-ylpyrimidin-5-yl)boronic acid (59.2 mg, 0.250 mmol) were suspended in a mixed solution of 2 M aqueous sodium carbonate solution (0.250 mL) and toluene (4 mL) and, after argon substitution, tris(dibenzylideneacetone)dipalladium (0) (7.62 mg, 0.00833 mmol) and 2-dicyclohexylphosphino-2′,6′-dimethoxybiphenyl (14.1 mg, 0.0330 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-65:35) to give tert-butyl [(3S)-7-(4,6-dimethyl-2-morpholin-4-ylpyrimidin-5-yl)-2,3-dihydro-1-benzofuran-3-yl]carbamate (79.1 mg, yield 89% (2 steps)) as a colorless oil. tert-Butyl [(3S)-7-(4,6-dimethyl-2-morpholin-4-ylpyrimidin-5-yl)-2,3-dihydro-1-benzofuran-3-yl]carbamate (79.1 mg, 0.185 mmol) obtained above was suspended in toluene (2 mL), and trifluoroacetic acid (0.5 mL) was added. The reaction mixture was stirred at room temperature for 30 min, and concentrated under reduced pressure. To the residue was added sodium hydrogen carbonate; and the mixture was extracted with ethyl acetate. The extract was dried over sodium sulfate, and concentrated under reduced pressure to give the title compound (58.5 mg, yield 97%) as a pale-yellow oil.

¹H NMR (300 MHz, DMSO-d₆) δ 2.03 (3H, s), 2.06 (3H, s), 2.24 (2H, br s), 3.60-3.76 (8H, m), 3.99 (1H, dd, J=8.3, 5.3 Hz), 4.50-4.67 (2H, m), 6.90-7.02 (2H, m), 7.34 (1H, dd, J=6.6, 1.7 Hz).

### Reference Example 70

### methyl {(3S)-6-[{(3S)-7-[(5-chloropyrimidin-2-yl)amino]-2,3-dihydro-1-benzofuran-3-yl}(trifluoroacetyl)amino]-2,3-dihydro-1-benzofuran-3-yl}acetate

A suspension of methyl [(3S)-6-{[(3S)-7-bromo-2,3-dihydro-1-benzofuran-3-yl](trifluoroacetyl)amino}-2,3-dihydro-1-benzofuran-3-yl]acetate (0.300 g, 0.6 mmol), 5-chloropyrimidine-2-amine (0.093 g, 0.72 mmol), tris(dibenzylideneacetone)dipalladium (0) (22 mg, 0.024 mmol), 4,5-bis(diphenylphosphino)-9,9-dimethylxanthene (27.8 mg, 0.048 mmol) and cesium carbonate (391 mg, 1.2 mmol) in toluene (8 mL) was stirred under an argon atmosphere at 105° C. for 16 hr. The reaction mixture was filtered through celite, and concentrated under reduced pressure. The residue was purified by silica gel column chromatography (hexane:ethyl acetate=95:5-70:30) to give the title compound (0.146 g, yield 44%) as a pale-yellow solid.

MS m/z 549 (M+H)⁺.

### Reference Example 71

### methyl {(3S)-6-[{(3S)-7-[(4-cyano-2-fluorophenyl)amino]-2,3-dihydro-1-benzofuran-3-yl}(trifluoroacetyl)amino]-2,3-dihydro-1-benzofuran-3-yl}acetate

A suspension of methyl [(3S)-6-{[(3S)-7-bromo-2,3-dihydro-1-benzofuran-3-yl](trifluoroacetyl)amino}-2,3-dihydro-1-benzofuran-3-yl]acetate (0.300 g, 0.6 mmol), 4-amino-3-fluorobenzonitrile (0.098 g, 0.72 mmol), tris(dibenzylideneacetone)dipalladium (0) (22 mg, 0.024 mmol), 4,5-bis(diphenylphosphino)-9,9-dimethylxanthene (27.8 mg, 0.048 mmol) and cesium carbonate (391 mg, 1.2 mmol) in toluene (8 mL) was stirred under an argon atmosphere at 105° C. for 16 hr. The reaction mixture was filtered through celite, and concentrated under reduced pressure. The residue was purified by silica gel column chromatography (hexane:ethyl acetate=95:5-70:30) to give the title compound (0.160 g, yield 48%) as a solid.

MS m/z 556 (M+H)⁺.

### Reference Example 72

### methyl {(3S)-6-[{(3S)-7-[(4-cyanophenyl)amino]-2,3-dihydro-1-benzofuran-3-yl}(trifluoroacetyl)amino]-2,3-dihydro-1-benzofuran-3-yl}acetate

A suspension of methyl [(3S)-6-{[(3S)-7-bromo-2,3-dihydro-1-benzofuran-3-yl](trifluoroacetyl)amino}-2,3-dihydro-1-benzofuran-3-yl]acetate (0.300 g, 0.6 mmol), 4-aminobenzonitrile (0.0851 g, 0.72 mmol), tris(dibenzylideneacetone)dipalladium (0) (22 mg, 0.024 mmol), 4,5-bis(diphenylphosphino)-9,9-dimethylxanthene (27.8 mg, 0.048 mmol) and cesium carbonate (391 mg, 1.2 mmol) in toluene (8 mL) was stirred overnight under an argon atmosphere at 105° C. The reaction mixture was filtered through celite, and concentrated under reduced pressure. The residue was purified by silica gel column chromatography (hexane:ethyl acetate=95:5-70:30) to give the title compound (0.316 g, yield 98%) as a solid.

MS m/z 538 (M+H)⁺.

### Reference Example 73

### methyl {(3S)-6-[{(3S)-7-[(3-cyanophenyl)amino]-2,3-dihydro-1-benzofuran-3-yl}(trifluoroacetyl)amino]-2,3-dihydro-1-benzofuran-3-yl}acetate

A suspension of methyl [(3S)-6-{[(3S)-7-bromo-2,3-dihydro-1-benzofuran-3-yl](trifluoroacetyl)amino}-2,3-dihydro-1-benzofuran-3-yl]acetate (0.400 g, 0.8 mmol), 3-aminobenzonitrile (0.113 g, 0.72 mmol), tris(dibenzylideneacetone)dipalladium (0) (29 mg, 0.032 mmol), 4,5-bis(diphenylphosphino)-9,9-dimethylxanthene (37 mg, 0.064 mmol) and cesium carbonate (521 mg, 1.6 mmol) in toluene (8 mL) was stirred under an argon atmosphere at 105° C. for 16 hr. The reaction mixture was filtered through celite, and concentrated under reduced pressure. The residue was purified by silica gel column chromatography (hexane:ethyl acetate=95:5-70:30) to give the title compound (0.331 g, yield 77%) as an oil.

MS m/z 538 (M+H)⁺.

### Reference Example 74

### methyl [(3S)-6-{[(3S)-7-(pyridin-3-ylamino)-2,3-dihydro-1-benzofuran-3-yl](trifluoroacetyl)amino}-2,3-dihydro-1-benzofuran-3-yl]acetate

A suspension of methyl [(3S)-6-{[(3S)-7-bromo-2,3-dihydro-1-benzofuran-3-yl](trifluoroacetyl)amino}-2,3-dihydro-1-benzofuran-3-yl]acetate (0.400 g, 0.8 mmol), pyridin-3-amine (90.4 mg, 0.96 mmol), tris(dibenzylideneacetone)dipalladium (0) (29 mg, 0.032 mmol), 4,5-bis(diphenylphosphino)-9,9-dimethylxanthene (37 mg, 0.064 mmol) and cesium carbonate (521 mg, 1.6 mmol) in toluene (8 mL) was stirred under an argon atmosphere at 105° C. for 16 hr. The reaction mixture was filtered through celite, and concentrated under reduced pressure. The residue was purified by silica gel column chromatography (hexane:ethyl acetate=70:30-50:50) to give the title compound (0.289 g, yield 70%) as an oil.

MS m/z 514 (M+H)⁺.

### Reference Example 75

### methyl {(3S)-6-[{(3S)-7-[(6-methylpyridin-3-yl)amino]-2,3-dihydro-1-benzofuran-3-yl}(trifluoroacetyl)amino]-2,3-dihydro-1-benzofuran-3-yl}acetate

A suspension of methyl [(3S)-6-{[(3S)-7-bromo-2,3-dihydro-1-benzofuran-3-yl](trifluoroacetyl)amino}-2,3-dihydro-1-benzofuran-3-yl]acetate (0.400 g, 0.8 mmol), 6-methylpyridin-3-amine (104 mg, 0.96 mmol), tris(dibenzylideneacetone)dipalladium (0) (29 mg, 0.032 mmol), 4,5-bis(diphenylphosphino)-9,9-dimethylxanthene (37 mg, 0.064 mmol) and cesium carbonate (521 mg, 1.6 mmol) in toluene (8 mL) was stirred under an argon atmosphere at 105° C. for 16 hr. The reaction mixture was filtered through celite, and concentrated under reduced pressure. The residue was purified by silica gel column chromatography (hexane:ethyl acetate=70:30-50:50) to give the title compound (0.219 g, yield 42%) as an oil.

MS m/z 528 (M+H)⁺.

### Reference Example 76

### methyl {(3S)-6-[{(3S)-7-[(6-fluoropyridin-3-yl)amino]-2,3-dihydro-1-benzofuran-3-yl}(trifluoroacetyl)amino]-2,3-dihydro-1-benzofuran-3-yl}acetate

A suspension of methyl [(3S)-6-{[(3S)-7-bromo-2,3-dihydro-1-benzofuran-3-yl](trifluoroacetyl)amino}-2,3-dihydro-1-benzofuran-3-yl]acetate (0.400 g, 0.8 mmol), 6-fluoropyridin-3-amine (107 mg, 0.96 mmol), tris(dibenzylideneacetone)dipalladium (0) (29 mg, 0.032 mmol), 4,5-bis(diphenylphosphino)-9,9-dimethylxanthene (37 mg, 0.064 mmol) and cesium carbonate (521 mg, 1.6 mmol) in toluene (8 mL) was stirred under an argon atmosphere at 105° C. for 16 hr. The reaction mixture was filtered through celite, and concentrated under reduced pressure. The residue was purified by silica gel column chromatography (hexane:ethyl acetate=70:30-50:50) to give the title compound (0.263 g, yield 62%) as an oil.

MS m/z 532 (M+H)⁺.

### Reference Example 77

### methyl [(3S)-6-{[(3S)-7-{[2-amino-3-(benzyloxy)phenyl]amino}-2,3-dihydro-1-benzofuran-3-yl](trifluoroacetyl)amino}-2,3-dihydro-1-benzofuran-3-yl]acetate

Methyl [(3S)-6-{[(3S)-7-bromo-2,3-dihydro-1-benzofuran-3-yl]amino}-2,3-dihydro-1-benzofuran-3-yl]acetate (1.0 g, 2.0 mmol), 3-(benzyloxy)-2-nitroaniline (1.0 g, 2.0 mmol), tris(dibenzylideneacetone)dipalladium (91 mg, 0.1 mmol), 2-(dicyclohexylphosphino)-2′,4′,6′-triisopropyl-1′,1′-biphenyl (95 mg, 0.2 mmol) and tripotassium phosphate (849 mg, 4.0 mmol) were suspended in toluene (10 mL), and the suspension was stirred under an argon atmosphere at 100° C. overnight. The suspension was allowed to cool, the mixture was diluted with water and extracted with ethyl acetate. The extract was filtered through a celite pad and the filtrate was concentrated. The residue was washed with toluene to give a yellow solid (700 mg, yield 53%). The obtained solid was suspended in ethanol (40 mL)-water (10 mL), reduced iron (2.2 g, 40 mmol) and ammonium chloride (2.1 g, 40 mmol) were added and the mixture was stirred at 90° C. for 4 hr. The reaction mixture was allowed to cool to room temperature, ethyl acetate was added and the mixture was filtered through a celite pad. The filtrate was extracted with ethyl acetate, and the extract was washed with aqueous sodium bicarbonate solution and saturated brine. The organic layer was dried over anhydrous magnesium sulfate, and the volatile component was evaporated under reduced pressure. The residue was purified by silica gel column chromatography (hexane:ethyl acetate=90:10-50:50) to give the title compound as a pale-yellow solid (580 mg, yield 87%).

MS m/z 634 (M+H)⁺.

### Reference Example 78

### methyl [(3S)-6-{[(3S)-7-(2-ethyl-4-hydroxy-1H-benzimidazol-1-yl)-2,3-dihydro-1-benzofuran-3-yl](trifluoroacetyl)amino}-2,3-dihydro-1-benzofuran-3-yl]acetate

Methyl [(3S)-6-{[(3S)-7-{[2-amino-3-(benzyloxy)phenyl]amino}-2,3-dihydro-1-benzofuran-3-yl](trifluoroacetyl)amino}-2,3-dihydro-1-benzofuran-3-yl]acetate (317 mg, 0.5 mmol) was dissolved in N,N-dimethylacetamide (10 mL), propanoyl chloride (56 mg, 0.6 mmol) was added, and the mixture was stirred for 30 min. The mixture was diluted with aqueous sodium bicarbonate solution and extracted with ethyl acetate. The extract was washed with saturated brine, dried over anhydrous magnesium sulfate, and the volatile component was evaporated under reduced pressure. Acetic acid (10 mL) was added to the residue, and the mixture was stirred at 90° C. overnight. Acetic acid was evaporated under reduced pressure, and the residue was diluted with aqueous sodium bicarbonate solution and extracted with ethyl acetate. The extract was washed with saturated brine, dried over anhydrous magnesium sulfate. To the residue were added methanol (10 mL) and 10% palladium-carbon (50% water-containing product, 100 mg), and the mixture was stirred under a hydrogen atmosphere for 1 hr. The catalyst was filtered through a celite pad and the filtrate was concentrated. The residue was purified by silica gel column chromatography (hexane:ethyl acetate=90:10-50:50) to give the title compound as a white solid (260 mg, yield 89%).

MS m/z 582 (M+H)⁺.

### Reference Example 79

### methyl {(3S)-6-[{(3S)-7-[2-ethyl-4-(2,2,2-trifluoroethoxy)-1H-benzimidazol-1-yl]-2,3-dihydro-1-benzofuran-3-yl}(trifluoroacetyl)amino]-2,3-dihydro-1-benzofuran-3-yl}acetate

Methyl [(3S)-6-{[(3S)-7-(2-ethyl-4-hydroxy-1H-benzimidazol-1-yl)-2,3-dihydro-1-benzofuran-3-yl](trifluoroacetyl)amino}-2,3-dihydro-1-benzofuran-3-yl]acetate (130 mg, 0.22 mmol), 2,2,2-trifluoroethyl methanesulfonate (59 mg, 0.33 mmol) and potassium carbonate (61 mg, 0.45 mmol) were suspended in N,N-dimethylformamide (2 mL), and the suspension was stirred at 80° C. for 2 hr. The suspension was allowed to cool, diluted with saturated brine, extracted with ethyl acetate and dried over anhydrous magnesium sulfate, and the volatile component was evaporated under reduced pressure. The residue was purified by silica gel column chromatography (hexane:ethyl acetate=70:30-20:80) to give the title compound (95 mg, yield 76%) as a colorless solid.

MS m/z 664 (M+H)⁺.

### Reference Example 80

### methyl [(3S)-6-{[(3S)-7-{2-ethyl-4-[3-(methylsulfonyl)propoxy]-1H-benzimidazol-1-yl}-2,3-dihydro-1-benzofuran-3-yl](trifluoroacetyl)amino}-2,3-dihydro-1-benzofuran-3-yl]acetate

Methyl [(3S)-6-{[(3S)-7-(2-ethyl-4-hydroxy-1H-benzimidazol-1-yl)-2,3-dihydro-1-benzofuran-3-yl](trifluoroacetyl)amino}-2,3-dihydro-1-benzofuran-3-yl]acetate (130 mg, 0.22 mmol), 3-(methylsulfonyl)propyl p-toluenesulfonate (98 mg, 0.33 mmol) and potassium carbonate (61 mg, 0.45 mmol) were suspended in N,N-dimethylformamide (2 mL), and the suspension was stirred at 80° C. for 2 hr. The suspension was allowed to cool, diluted with saturated brine, extracted with ethyl acetate and dried over anhydrous magnesium sulfate, and the volatile component was evaporated under reduced pressure. The residue was purified by silica gel column chromatography (hexane:ethyl acetate=70:30-20:80) to give the title compound (110 mg, yield 83%) as a colorless solid.

MS m/z 702 (M+H)⁺.

### Reference Example 81

### methyl {(3S)-6-[{(3S)-7-[6-(benzyloxy)-2-methyl-1H-benzimidazol-1-yl]-2,3-dihydro-1-benzofuran-3-yl}(trifluoroacetyl)amino]-2,3-dihydro-1-benzofuran-3-yl}acetate

Methyl [(3S)-6-{[(3S)-7-bromo-2,3-dihydro-1-benzofuran-3-yl](trifluoroacetyl)amino}-2,3-dihydro-1-benzofuran-3-yl]acetate (195 mg, 0.8 mmol), 5-(benzyloxy)-2-nitroaniline (400 mg, 0.8 mmol), tris(dibenzylideneacetone)dipalladium (0) (37 mg, 0.04 mmol), 2-(dicyclohexylphosphino)-2′,4′,6′-triisopropyl-1′,1′-biphenyl (38 mg, 0.08 mmol), tripotassium phosphate (340 mg, 1.6 mmol) were suspended in toluene (5 mL), and the suspension was stirred overnight under an argon atmosphere at 100° C. The suspension was allowed to cool, diluted with saturated brine, and extracted with ethyl acetate. The extract was dried over anhydrous magnesium sulfate, and the volatile component was evaporated under reduced pressure. The residue was purified by silica gel column chromatography (hexane:ethyl acetate=70:30-20:80) to give an oil (640 mg). The obtained compound was suspended in ethanol (30 mL), water (5 mL) and acetic acid (5 mL), reduced iron (440 mg, 7.8 mmol) and calcium chloride (870 mg, 7.8 mmol) were added and the mixture was stirred at 60° C. for 2 hr. The reaction mixture was allowed to cool to room temperature, the volatile component was evaporated under reduced pressure, and the residue was extracted with ethyl acetate, and the extract was washed with aqueous sodium bicarbonate solution and saturated brine. The organic layer was dried over anhydrous magnesium sulfate, and the volatile component was evaporated under reduced pressure. The residue was purified by silica gel column chromatography (hexane:ethyl acetate=50:50-0:100) to give the title compound as an oil compound (210 mg, yield 40%).

MS m/z 658 (M+H)⁺.

### Reference Example 82

### methyl {(3S)-6-[{(3S)-7-[2-methyl-6-(2,2,2-trifluoroethoxy)-1H-benzimidazol-1-yl]-2,3-dihydro-1-benzofuran-3-yl}(trifluoroacetyl)amino]-2,3-dihydro-1-benzofuran-3-yl}acetate

To methyl {(3S)-6-[{(3S)-7-[6-(benzyloxy)-2-methyl-1H-benzimidazol-1-yl]-2,3-dihydro-1-benzofuran-3-yl}(trifluoroacetyl)amino]-2,3-dihydro-1-benzofuran-3-yl}acetate (210 mg, 0.40 mmol) were added methanol (20 mL) and 10% palladium-carbon (50% water-containing product, 50 mg), and the mixture was stirred under a hydrogen atmosphere overnight. The catalyst was filtered off and the filtrate was concentrated. The obtained residue (150 mg), 2,2,2-trifluoroethyl methanesulfonate (59 mg, 0.33 mmol), potassium carbonate (61 mg, 0.45 mmol) were suspended in N,N-dimethylformamide (2 mL), and the suspension was stirred at 60° C. for 3 hr. The suspension was allowed to cool, diluted with saturated brine, extracted with ethyl acetate and dried over anhydrous magnesium sulfate, and the volatile component was evaporated under reduced pressure. The residue was purified by silica gel column chromatography (hexane:ethyl acetate=70:30-0:100) to give the title compound (150 mg, yield 62%) as a pale-yellow solid.

MS m/z 650 (M+H)⁺.

### Reference Example 83

### 2-chloro-2′,6′-dimethylbiphenyl-3-carbaldehyde

To a solution of 2-chloro-3-hydroxybenzaldehyde (1.57 g, 10.0 mmol) in pyridine (30 mL) was added dropwise trifluoromethanesulfonic anhydride (2.02 mL, 12.0 mmol) under a nitrogen atmosphere at 0° C., and the mixture was warmed to room temperature and stirred for 0.5 hr. The solvent was evaporated under reduced pressure, and the residue was diluted with ethyl acetate. The mixture was washed successively with 1N hydrochloric acid and saturated brine, dried over anhydrous magnesium sulfate, and concentrated under reduced pressure. The residue was purified by silica gel column chromatography (ethyl acetate:hexane=0:100-25:75) to give 2-chloro-3-formylphenyl trifluoromethanesulfonate (2.65 g, yield 92%) as a colorless oil. This product (2.65 g, 9.18 mmol), 2,6-dimethylphenylboronic acid (1.65 g, 11.0 mmol) and tripotassium phosphate (3.90 g, 18.4 mmol) was dissolved in a mixed solution of tetrahydrofuran (50 mL) and water (10 mL), palladium (II) acetate (62 mg, 0.275 mmol) and 2-dicyclohexylphosphino-2′,6′-dimethoxybiphenyl (0.226 g, 0.551 mmol) were added, and the mixture was stirred under an argon atmosphere at 800° C. for 21 hr. Ethyl acetate and water were added to the reaction mixture, and the insoluble material was filtered off through celite. The organic layer of the filtrate was washed with saturated brine, dried over anhydrous magnesium sulfate, and concentrated under reduced pressure. The residue was purified by silica gel column chromatography (ethyl acetate:hexane=0:100-15:85) and the obtained solid was washed with ethyl acetate-heptane to give colorless crystals (0.393 g). The mother liquor was purified by preparative HPLC to give colorless crystals (0.292 g). total 0.685 g (yield 31%).

¹H NMR (300 MHz, CDCl₃) δ 2.00 (6H, s), 7.13-7.18 (2H, m), 7.22-7.28 (1H, m), 7.38-7.43 (1H, m), 7.45-7.51 (1H, m), 7.96 (1H, dd, J=7.5, 1.9 Hz), 10.58 (1H, d, J=0.8 Hz).

### Reference Example 84

### 2-hydroxy-2′,6′-dimethylbiphenyl-3-carbaldehyde

To a solution of 3-bromo-2-hydroxybenzaldehyde (1.12 g, 5.57 mmol), 2,6-dimethylphenylboronic acid (1.00 g, 6.69 mmol) and tripotassium phosphate (3.55 g, 16.7 mmol) in toluene (30 mL) and water (6 mL) were added palladium (II) acetate (38 mg, 0.167 mmol) and 2-dicyclohexylphosphino-2′,6′-dimethoxybiphenyl (0.137 g, 0.334 mmol), and the mixture was stirred under an argon atmosphere at 100° C. for 17 hr. Ethyl acetate and water were added to the reaction mixture, and the insoluble material was filtered off through celite. The organic layer of the filtrate was washed with saturated brine, dried over anhydrous magnesium sulfate, and concentrated under reduced pressure. The residue was purified by silica gel column chromatography (ethyl acetate:hexane=0:100-20:80) to give the title compound (1.18 g, yield 94%) as a brown oil.

MS (ESI−) m/z 225 (M−H)⁻.

### Reference Example 85

### methyl 3-bromo-2-methylbenzoate

To a suspension of 3-bromo-2-methylbenzoic acid (3.23 g, 41.6 mmol) in methanol (15 mL) was added dropwise concentrated sulfuric acid (1.60 mL, 30.0 mmol) at room temperature, and the reaction mixture was heated at 50° C. for 20 hr. The reaction mixture was concentrated under reduced pressure, and the residue was diluted with diethyl ether, washed successively with water, saturated aqueous sodium hydrogen carbonate and saturated brine, and concentrated under reduced pressure. The residue was purified by silica gel column chromatography (ethyl acetate:hexane=0:100-25:75) to give the title compound (3.20 g, yield 93%) as colorless crystals.

¹H NMR (300 MHz, CDCl₃) δ 2.63 (3H, s), 3.90 (3H, s), 7.06-7.13 (1H, m), 7.67-7.75 (2H, m).

### Reference Example 86

### methyl 3-[1-(difluoromethyl)-3,5-dimethyl-1H-pyrazol-4-yl]-2-methylbenzoate

In the same manner as in Reference Example 84, the title compound was obtained as colorless crystals from methyl 2-bromo-3-methylbenzoate and 1-(difluoromethyl)-3,5-dimethyl-4-(4,4,5,5-tetramethyl-1,3,2-dioxaborolan-2-yl)-1H-pyrazole. yield 83%.

MS m/z 295 (M+H)⁺.

### Reference Example 87

### {3-[1-(difluoromethyl)-3,5-dimethyl-1H-pyrazol-4-yl]-2-methylphenyl}methanol

Methyl 3-[1-(difluoromethyl)-3,5-dimethyl-1H-pyrazol-4-yl]-2-methylbenzoate (2.18 g, 7.41 mmol) was dissolved in tetrahydrofuran (30 mL), 1.5 M diisobutylaluminum hydride toluene solution (14.8 mL, 22.2 mmol) was added dropwise under a nitrogen atmosphere at 0° C. and the mixture was stirred for 16 hr. To the reaction mixture was added sodium sulfate 10 hydrate (7.15 g, 22.2 mmol), and the mixture was stirred at room temperature for 2 hr. Insoluble material was filtered off through celite, and the filtrate was concentrated under reduced pressure. The residue was purified by silica gel column chromatography (ethyl acetate:hexane=10:90-50:50) to give the title compound (1.82 g, yield 92%) as a colorless oil.

MS m/z 267 (M+H)⁺.

### Reference Example 88

### methyl [(3S)-6-{[2-methyl-3-(4,4,5,5-tetramethyl-1,3,2-dioxaborolan-2-yl)benzyl]amino}-2,3-dihydro-1-benzofuran-3-yl]acetate

To a solution of methyl {(3S)-6-[(3-bromo-2-methylbenzyl)amino]-2,3-dihydro-1-benzofuran-3-yl}acetate (0.548 g, 1.40 mmol), bis(pinacolato)diboron (0.713 g, 2.81 mmol) and potassium acetate (0.482 g, 4.91 mmol) in N,N-dimethylformamide (7 mL) was added [1,1′-bis(diphenylphosphino)ferrocene]palladium dichloride (31 mg, 0.042 mmol), and the mixture was stirred under an argon atmosphere at 100° C. for 15 hr. Ethyl acetate and water were added to the reaction mixture, and the insoluble material was filtered off through celite. The organic layer of the filtrate was washed with saturated brine, dried over anhydrous magnesium sulfate, and concentrated under reduced pressure. The residue was purified by silica gel column chromatography (ethyl acetate:hexane=5:95-40:60) to give the title compound as a yellow viscous oil.

MS m/z 438 (M+H)⁺.

### Reference Example 89

### 5-bromo-4,6-dimethylpyrimidin-2-ol

To a solution of 2-hydroxy-4,6-dimethylpyrimidine (13.5 g, 109 mmol) in N,N-dimethylformamide (200 mL) was added N-bromosuccinimide (20.3 g, 114 mmol) by small portions, and the mixture was stirred at room temperature for 3 days. The solvent was evaporated under reduced pressure, and the residue was suspended in ethyl acetate and heated. The suspension was cooled and the precipitate was collected by filtration, and washed with ethanol to give the title compound (11.9 g, yield 54%) as dark gray crystals.

MS m/z 203 (M+H)⁺.

### Reference Example 90

### 5-bromo-2-(difluoromethoxy)-4,6-dimethylpyrimidine

5-Bromo-4,6-dimethylpyrimidin-2-ol (2.03 g, 10.0 mmol), sodium chlorodifluoroacetate (3.05 g, 20.0 mmol), and potassium carbonate (1.73 g, 12.5 mmol) were added to a mixed solution of N,N-dimethylformamide (20 mL) and water (3 mL), and the mixture was stirred under a nitrogen atmosphere at 100° C. for 16 hr. The solvent was evaporated under reduced pressure, to the residue were added ethyl acetate and water, and the insoluble material was filtered off through celite. The organic layer of the filtrate was washed with saturated brine, dried over anhydrous sodium sulfate, and concentrated under reduced pressure. The residue was purified by silica gel column chromatography (ethyl acetate:hexane=0:100-20:80) to give the title compound (0.516 g, yield 20%) as a yellow oil.

MS m/z 253 (M+H)⁺.

### Reference Example 91

### [4-(methoxymethoxy)-2,6-dimethylphenyl]boronic acid

According to WO2008/001931, the title compound was synthesized. yield 91%.

### Reference Example 92

### methyl 4′-(methoxymethoxy)-2,2′,6′-trimethylbiphenyl-3-carboxylate

In the same manner as in Reference Example 84, the title compound was obtained as a yellow oil from methyl 3-bromo-2-methylbenzoate and [4-(methoxymethoxy)-2,6-dimethylphenyl]boronic acid. yield 94%.

¹H NMR (300 MHz, CDCl₃) δ 1.89 (6H, s), 2.19 (3H, s), 3.52 (3H, s), 3.91 (3H, s), 5.20 (2H, s), 6.80 (2H, s), 7.14-7.20 (1H, m), 7.25-7.33 (1H, m), 7.83 (1H, dd, J=7.6, 1.5 Hz).

### Reference Example 93

### methyl 4′-hydroxy-2,2′,6′-trimethylbiphenyl-3-carboxylate

To a solution of methyl 4′-(methoxymethoxy)-2,2′,6′-trimethylbiphenyl-3-carboxylate (4.14 g, 13.2 mmol) in methanol (60 mL) was added dropwise 5-10% hydrogen chloride/methanol solution (6 mL), and the mixture was stirred at room temperature for 16 hr. The solvent was evaporated under reduced pressure, and the residue was treated with saturated aqueous sodium hydrogen carbonate, and the mixture was extracted with ethyl acetate. The extract was washed with saturated brine, and concentrated under reduced pressure. The residue was purified by silica gel column chromatography (ethyl acetate:hexane=5:95-40:60) to give the title compound (3.40 g, yield 96%) as a colorless viscous oil.

MS m/z 271 (M+H)⁺.

### Reference Example 94

### methyl 2,2′,6′-trimethyl-4′-[3-(methylsulfonyl)propoxy]biphenyl-3-carboxylate

To a solution of methyl 4′-hydroxy-2,2′,6′-trimethylbiphenyl-3-carboxylate (1.24 g, 4.59 mmol) and 3-(methylsulfonyl)propyl 4-methylbenzenesulfonate (1.41 g, 4.82 mmol) synthesized according to WO2008/001931 in N,N-dimethylformamide (10 mL) was added potassium carbonate (0.761 g, 5.50 mmol), and the mixture was stirred under a nitrogen atmosphere at 80° C. for 28 hr. Water was added to the reaction mixture, and the mixture was extracted with ethyl acetate. The extract was washed with saturated brine, dried over anhydrous magnesium sulfate, and concentrated under reduced pressure. The residue was purified by silica gel column chromatography (ethyl acetate:hexane=30:70-70:30) to give the title compound (1.59 g, yield 89%) as a colorless oil.

MS m/z 391 (M+H)⁺.

### Reference Example 95

### {2,2′,6′-trimethyl-4′-[3-(methylsulfonyl)propoxy]biphenyl-3-yl}methanol

In the same manner as in Reference Example 87, the title compound was obtained as colorless crystals from methyl 2,2′,6′-trimethyl-4′-[3-(methylsulfonyl)propoxy]biphenyl-3-carboxylate. yield 87%.

MS m/z 363 (M+H)⁺.

### Reference Example 96

### methyl 3-(2,5-dimethylthiophen-3-yl)-2-fluorobenzoate

To a solution of methyl 3-bromo-2-fluorobenzoate (300 mg, 1.29 mmol) and (2,5-dimethylthiophen-3-yl)boronic acid (402 mg, 2.57 mmol) and 2 M aqueous sodium carbonate solution (1.29 mL, 2.58 mmol) in toluene (6.5 mL) were added under an argon atmosphere tris(dibenzylideneacetone)dipalladium (0) (47.3 mg, 0.0516 mmol) and dicyclohexyl(2′,6′-dimethoxybiphenyl-2-yl)phosphane (84.7 mg, 0.206 mmol), and the mixture was stirred at 100° C. overnight The reaction mixture was cooled to room temperature, filtered through celite, and concentrated under reduced pressure. The residue was purified by silica gel column chromatography (hexane:ethyl acetate=100:0-90:10) to give the title compound (354 mg, yield 100%) as a colorless oil.

¹H NMR (300 MHz, CDCl₃) δ 2.32 (3H, s), 2.44 (3H, s), 3.94 (3H, s), 6.64 (1H, s), 7.22 (1H, t, J=7.7 Hz), 7.39-7.50 (1H, m), 7.83-7.92 (1H, m).

### Reference Example 97

### [3-(2,5-dimethylthiophen-3-yl)-2-fluorophenyl]methanol

To a solution of methyl [3-(2,5-dimethylthiophen-3-yl)-2-fluoro]benzoate (354 mg, 1.29 mmol) in tetrahydrofuran (6.5 mL) was added under ice-cooling diisobutylaluminum hydride (1.5 M toluene solution, 3.44 mL, 5.16 mmol), and the mixture was stirred for 1 hr. Sodium sulfate 10 hydrate (1.66 g, 5.16 mmol) was slowly added, and the mixture was stirred for 30 min at room temperature. Insoluble material was filtered off, and the filtrate was concentrated under reduced pressure. The residue was purified by silica gel column chromatography (hexane:ethyl acetate=95:5-67:33) to give the title compound (311 mg, yield 100%) as a colorless oil.

¹H NMR (300 MHz, CDCl₃) δ 1.81 (1H, t, J=6.2 Hz), 2.33 (3H, s), 2.44 (3H, s), 4.81 (2H, d, J=6.1 Hz), 6.64 (1H, s), 7.11-7.25 (2H, m), 7.38 (1H, td, J=7.1, 2.1 Hz).

### Reference Example 98

### methyl 3-{[2-(acetylamino)phenyl]amino}-2-methylbenzoate

N-(2-Aminophenyl)acetamide (750 mg, 5.0 mmol), methyl 3-bromo-2-methylbenzoate (1.1 g, 5.0 mmol), tris(dibenzylideneacetone)dipalladium (230 mg, 0.25 mmol), 4,5-bis(diphenylphosphino)-9,9-dimethylxanthene (290 mg, 0.5 mmol) and cesium carbonate (3.2 g, 10 mmol) were suspended in toluene (25 mL), and the mixture was stirred under an argon atmosphere at 100° C. for 4 hr. The mixture was allowed to cool, diluted with water and extracted with ethyl acetate. The extract was washed with saturated brine and dried over anhydrous magnesium sulfate, and the volatile component was evaporated under reduced pressure. The residue was purified by silica gel column chromatography (hexane:ethyl acetate=70:30-20:80) to give the title compound as a pale-yellow solid (410 mg, yield 27%).

¹H NMR (300 MHz, DMSO-d₆) δ 2.05 (3H, s), 2.29 (3H, s), 3.83 (3H, s), 6.85-7.42 (8H, m), 9.60 (1H, s).

### Reference Example 99

### [2-methyl-3-(2-methyl-1H-benzimidazol-1-yl)phenyl]methanol

Methyl 3-{[2-(acetylamino)phenyl]amino}-2-methylbenzoate (410 mg, 1.37 mmol) was dissolved in acetic acid (15 mL), and the mixture was stirred at 100° C. overnight. The reaction mixture was allowed to cool, and the volatile component was evaporated under reduced pressure. The residue was dissolved in tetrahydrofuran (20 mL), lithium aluminum hydride (6.9 mmol, 260 mg) was added at 0° C., and the mixture was stirred at room temperature for 30 min. Sodium sulfate 10 hydrate was added, and then ethyl acetate was added, and the mixture was filtered through a celite pad. The filtrate was concentrated under reduced pressure and the obtained residue was purified by silica gel column chromatography (hexane:ethyl acetate=70:30-20:80) to give the title compound (300 mg, yield 86%) as a colorless solid.

¹H NMR (300 MHz, DMSO-d₆) δ 1.77 (3H, s), 2.28 (3H, s), 4.60 (2H, d, J=4.9 Hz), 5.29 (1H, t, J=5.3 Hz), 6.83 (1H, d, J=7.2 Hz), 7.10-7.31 (3H, m), 7.43 (1H, t, J=7.8 Hz), 7.63 (2H, d, J=7.2 Hz).

### Reference Example 100

### methyl [(3S)-6-{[2-methyl-3-(2-methyl-1H-benzimidazol-1-yl)benzyl] [(2-nitrophenyl)sulfonyl]amino}-2,3-dihydro-1-benzofuran-3-yl]acetate

To a solution of [2-methyl-3-(2-methyl-1H-benzimidazol-1-yl)phenyl]methanol (150 mg, 0.6 mmol), methyl [(3S)-6-{[(2-nitrophenyl)sulfonyl]amino}-2,3-dihydro-1-benzofuran-3-yl]acetate (280 mg, 0.72 mmol) and triphenylphosphine (240 mg, 0.9 mmol) in tetrahydrofuran (15 mL) was added a 2.2 M solution (0.41 mL, 0.9 mmol) of diethyl azodicarboxylate in toluene, and the mixture was stirred overnight at room temperature and diluted with saturated brine, and the mixture was extracted with ethyl acetate. The extract was dried over anhydrous magnesium sulfate, and the volatile component was evaporated under reduced pressure. The residue was purified by silica gel column chromatography (hexane:ethyl acetate=50:50-0:100) to give the title compound (350 mg, yield 93%) as a solid.

MS m/z 627 (M+H)⁺.

### Reference Example 101

### methyl 2-methyl-3-[2-methyl-6-(trifluoromethyl)-1H-benzimidazol-1-yl]benzoate

### methyl 2-methyl-3-[2-methyl-5-(trifluoromethyl)-1H-benzimidazol-1-yl]benzoate

4-(Trifluoromethyl)benzene-1,2-diamine (830 mg, 5.0 mmol) and acetic acid (300 mg, 5.0 mmol) were dissolved in N,N-dimethylformamide (10 mL), o-(7-azabenzotriazol-1-yl)-N,N,N′,N′-tetramethyluronium hexafluorophosphate (2.3 g, 6.0 mmol) and N,N-diisopropylethylamine (1.3 g, 10 mmol) were added, and the mixture was stirred at room temperature for 1 hr. The mixture was diluted with aqueous sodium bicarbonate solution and extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=50:50-0:100) to give an oil (1.6 g). The resultant product, methyl 3-bromo-2-methylbenzoate (1.1 g, 5.0 mmol), tris(dibenzylideneacetone)dipalladium (230 mg, 0.25 mmol), 4,5-bis(diphenylphosphino)-9,9-dimethylxanthene (290 mg, 0.5 mmol) and cesium carbonate (3.2 g, 10 mmol) were suspended in toluene (50 mL), and the mixture was stirred under an argon atmosphere at 100° C. overnight. The reaction mixture was allowed to cool, diluted with saturated brine, and the mixture was extracted with ethyl acetate. The extract was dried over anhydrous magnesium sulfate, and the volatile component was evaporated under reduced pressure. The obtained residue was purified by silica gel column chromatography (hexane:ethyl acetate=50:50-0:100) to give methyl 2-methyl-3-[2-methyl-6-(trifluoromethyl)-1H-benzimidazol-1-yl]benzoate (140 mg, yield 40%) and methyl 2-methyl-3-[2-methyl-5-(trifluoromethyl)-1H-benzimidazol-1-yl]benzoate (140 mg, yield 40%) each as a pale-yellow oil. methyl 2-methyl-3-[2-methyl-6-(trifluoromethyl)-1H-benzimidazol-1-yl]benzoate

¹H NMR (300 MHz, DMSO-d₆) δ 2.02 (3H, s), 2.34 (3H, s), 3.89 (3H, s), 7.21 (1H, s), 7.54-7.64 (2H, m), 7.72 (1H, dd, J=7.9, 1.3 Hz), 7.87 (1H, d, J=8.3 Hz), 8.06 (1H, dd, J=7.7, 1.3 Hz).

### methyl 2-methyl-3-[2-methyl-5-(trifluoromethyl)-1H-benzimidazol-1-yl]benzoate

¹H NMR (300 MHz, DMSO-d₆) δ 2.02 (3H, s), 2.35 (3H, s), 3.89 (3H, s), 7.10 (1H, d, J=8.7 Hz), 7.51 (1H, d, J=9.4 Hz), 7.61 (1H, t, J=7.7 Hz), 7.72 (1H, d, J=6.8 Hz), 8.00-8.08 (2H, m).

### Reference Example 102

### methyl 3-{[2-(acetylamino)phenyl]amino}-2-fluorobenzoate

In the same manner as in Reference Example 98, the title compound (180 mg, yield 12%) was synthesized from methyl 3-bromo-2-fluorobenzoate (1.17 g, 5.0 mmol).

¹H NMR (300 MHz, DMSO-d₆) δ 2.03 (3H, s), 3.86 (3H, s), 7.00-7.29 (6H, m), 7.44-7.51 (2H, m), 9.65 (1H, s).

### Reference Example 103

### methyl 2-fluoro-3-(2-methyl-1H-benzimidazol-1-yl)benzoate

A solution of methyl 3-{[2-(acetylamino)phenyl]amino}-2-fluorobenzoate (180 mg, 0.6 mmol) in acetic acid (20 mL) was stirred overnight at 100° C. The volatile component was evaporated under reduced pressure, and the residue was diluted with aqueous sodium bicarbonate solution and the mixture was extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=50:50-0:100) to give the title compound (130 mg, yield 76%).

¹H NMR (300 MHz, CDCl₃) δ 2.49 (3H, s), 3.98 (3H, s), 7.03 (1H, d, J=8.3 Hz), 7.18-7.24 (1H, m), 7.28-7.32 (1H, m), 7.40-7.48 (1H, m), 7.56-7.64 (1H, m), 7.76 (1H, d, J=7.6 Hz), 8.10-8.18 (1H, m).

### Reference Example 104

### methyl 3-[(5-fluoro-2-nitrophenyl)amino]-2-methylbenzoate

2-Bromo-4-fluoro-1-nitrobenzene (1.32 g, 6.0 mmol), methyl 3-amino-2-methylbenzoate (826 mg, 5.0 mmol), tris(dibenzylideneacetone)dipalladium (230 mg, 0.25 mmol), 2,2′-bis(diphenylphosphino)diphenyl ether (269 mg, 0.5 mmol) and tripotassium phosphate (3.2 g, 15 mmol) were suspended in toluene (25 mL), and the suspension was stirred under an argon atmosphere at 110° C. for 3 hr. The reaction mixture was allowed to cool, diluted with saturated brine, and the mixture was extracted with ethyl acetate. The extract was dried over anhydrous magnesium sulfate, and the volatile component was evaporated under reduced pressure. The obtained residue was purified by silica gel column chromatography (hexane:ethyl acetate=95:5-60:40) to give the title compound (1.4 g, yield 77%) as a pale-yellow solid.

¹H NMR (300 MHz, DMSO-d₆) δ 2.34 (3H, s), 3.86 (3H, s), 6.16 (1H, dd, J=11.7, 2.6 Hz), 6.68 (1H, ddd, J=9.7, 7.3, 2.8 Hz), 7.31-7.86 (3H, m), 8.26 (1H, dd, J=9.5, 6.1 Hz), 9.60 (1H, s).

### Reference Example 105

### methyl 3-[(2-amino-5-fluorophenyl)amino]-2-methylbenzoate

Methyl 3-[(5-fluoro-2-nitrophenyl)amino]-2-methylbenzoate (1.4 g, 4.6 mmol) was suspended in methanol (50 mL), platinum oxide (227 mg, 1 mmol) was added, and the mixture was stirred under a hydrogen atmosphere at room temperature overnight. Platinum oxide was filtered through a celite pad and the filtrate was concentrated. The residue was purified by silica gel column chromatography (hexane:ethyl acetate=90:10-30:70) to give the title compound (880 mg, yield 69%) as a pale-yellow oil.

¹H NMR (300 MHz, DMSO-d₆) δ 2.32 (3H, s), 3.82 (3H, s), 4.63 (2H, s), 6.44 (1H, dd, J=10.5, 2.8 Hz), 6.62 (1H, dd, J=8.5, 2.8 Hz), 6.66-6.75 (2H, m), 6.81 (1H, dd, J=7.9, 1.1 Hz), 7.14 (1H, t, J=7.8 Hz), 7.20-7.26 (1H, m).

### Reference Example 106

### methyl 3-(6-fluoro-2-methyl-1H-benzimidazol-1-yl)-2-methylbenzoate

To a solution of methyl 3-[(2-amino-5-fluorophenyl)amino]-2-methylbenzoate (880 mg, 3.20 mmol) in N,N-dimethylacetamide (20 mL) was added acetyl chloride (299 mg, 3.81 mmol), and the mixture was stirred at room temperature for 30 min. The mixture was diluted with aqueous sodium bicarbonate solution and extracted with ethyl acetate. The extract was washed with saturated brine, dried over anhydrous magnesium sulfate, and the volatile component was evaporated under reduced pressure. The residue was dissolved in acetic acid (20 mL), and the obtained solution was stirred at 120° C. for 2 days. The mixture was allowed to cool, and the volatile component was evaporated under reduced pressure. The obtained residue was diluted with aqueous sodium bicarbonate solution, and the mixture was extracted with ethyl acetate. The extract was dried over anhydrous magnesium sulfate, and the volatile component was evaporated under reduced pressure to give the title compound (850 mg, yield 89%) as a solid.

MS m/z 299 (M+H)⁺.

### Reference Example 107

### methyl 3-[(2-aminophenyl)amino]-2-methylbenzoate

1-Bromo-2-nitrobenzene (15.0 g, 74.2 mmol), methyl 3-amino-2-methylbenzoate (11.2 g, 67.5 mmol), tris(dibenzylideneacetone)dipalladium (2.47 g, 2.7 mmol), 2,2′-bis(diphenylphosphino)diphenyl ether (2.90 g, 5.4 mmol) and tripotassium phosphate (43 g, 203 mmol) were suspended in toluene (300 mL), and the mixture was stirred under an argon atmosphere at 100° C. overnight. The reaction mixture was allowed to cool, diluted with ethyl acetate-hexane (1:1) solution, and the mixture was filtered through a silica pad. The filtrate was concentrated and the obtained residue was suspended in methanol (300 mL). 10% Palladium-carbon (50% water-containing product, 5000 mg) was added, and the mixture was stirred under a hydrogen atmosphere overnight. The catalyst was filtered through a celite pad and the filtrate was concentrated. The residue was purified by silica gel column chromatography (hexane:ethyl acetate=90:10-40:60) to give the title compound (18.4 g, yield 96%) as an oil.

¹H NMR (300 MHz, DMSO-d₆) δ 2.34 (3H, s), 3.82 (3H, s), 4.74 (2H, s), 6.50-6.62 (3H, m), 6.72-6.82 (2H, m), 6.83-6.91 (1H, m), 7.00-7.11 (2H, m).

### Reference Example 108

### methyl 3-(2-ethoxy-1H-benzimidazol-1-yl)-2-methylbenzoate

Methyl 3-[(2-aminophenyl)amino]-2-methylbenzoate (513 mg, 2.0 mmol) and tetraethoxymethane (1.15 g, 6.0 mmol) were stirred in acetic acid (10 mL) at 60° C. for 1 hr. The reaction mixture was allowed to cool, and the volatile component was evaporated under reduced pressure. An aqueous sodium bicarbonate solution was added, and the mixture was extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=90:10-40:60) to give the title compound (460 mg, yield 74%) as a solid.

¹H NMR (300 MHz, DMSO-d₆) δ 1.32 (3H, t, J=7.0 Hz), 2.10 (3H, s), 3.88 (3H, s), 4.46-4.64 (2H, m), 6.81 (1H, d, J=7.6 Hz), 7.04-7.11 (1H, m), 7.13-7.21 (1H, m), 7.53 (2H, dd, J=10.0, 7.8 Hz), 7.60-7.66 (1H, m), 7.93-7.99 (1H, m).

### Reference Example 109

### methyl 3-[(2-amino-4-methoxyphenyl)amino]-2-methylbenzoate

In the same manner as in Reference Example 107, the title compound was synthesized using 1-bromo-4-methoxy-2-nitrobenzene.

¹H NMR (300 MHz, DMSO-d₆) δ 2.34 (3H, s), 3.68 (3H, s), 3.81 (3H, s), 4.79 (2H, s), 6.15 (1H, dd, J=8.5, 2.8 Hz), 6.33-6.41 (2H, m), 6.45 (1H, s), 6.78 (1H, d, J=8.5 Hz), 6.94-7.02 (2H, m).

### Reference Example 110

### methyl 3-[(2-amino-4-methylphenyl)amino]-2-methylbenzoate

In the same manner as in Reference Example 107, the title compound was synthesized using 1-bromo-4-methyl-2-nitrobenzene.

¹H NMR (300 MHz, DMSO-d₆) δ 2.18 (3H, s), 2.33 (3H, s), 3.81 (3H, s), 4.67 (2H, br s), 6.36 (1H, dd, J=7.7, 1.5 Hz), 6.44-6.52 (2H, m), 6.57 (1H, d, J=1.5 Hz), 6.71 (1H, d, J=7.7 Hz), 6.96-7.05 (2H, m).

### Reference Example 111

### methyl 3-[(2-amino-6-methylphenyl)amino]-2-methylbenzoate

In the same manner as in Reference Example 107, the title compound was synthesized using 1-bromo-6-methyl-2-nitrobenzene.

¹H NMR (300 MHz, DMSO-d₆) δ 1.97 (3H, s), 2.40 (3H, s), 3.81 (3H, s), 4.76 (2H, s), 6.09-6.15 (1H, m), 6.42 (1H, s), 6.48 (1H, d, J=6.8 Hz), 6.62 (1H, d, J=8.0 Hz), 6.86-6.95 (3H, m).

### Reference Example 112

### methyl 3-[(2-amino-3-methylphenyl)amino]-2-methylbenzoate

In the same manner as in Reference Example 107, the title compound was synthesized using 1-bromo-3-methyl-2-nitrobenzene.

¹H NMR (300 MHz, DMSO-d₆) δ 2.13 (3H, s), 2.35 (3H, s), 3.82 (3H, s), 4.47 (2H, s), 6.46-6.54 (2H, m), 6.61 (1H, s), 6.69 (1H, d, J=6.8 Hz), 6.81 (1H, d, J=7.2 Hz), 6.97-7.09 (2H, m).

### Reference Example 113

### methyl 3-[(2-amino-5-methoxyphenyl)amino]-2-methylbenzoate

In the same manner as in Reference Example 107, the title compound (yield 79%) was synthesized using 2-iodo-4-methoxy-1-nitrobenzene as aromatic halide.

¹H NMR (300 MHz, DMSO-d₆) δ 2.33 (3H, s), 3.59 (3H, s), 3.82 (3H, s), 4.31 (2H, s), 6.37 (1H, d, J=3.0 Hz), 6.45-6.50 (1H, m), 6.60-6.73 (3H, m), 7.04-7.16 (2H, m).

### Reference Example 114

### methyl 3-(6-methoxy-2-methyl-1H-benzimidazol-1-yl)-2-methylbenzoate

A solution of methyl 3-[(2-amino-5-methoxyphenyl)amino]-2-methylbenzoate (573 mg, 2 mmol) in acetic acid (10 mL)-acetic anhydride (5 mL) was stirred overnight at 100° C. The volatile component was evaporated under reduced pressure, an aqueous sodium bicarbonate solution was added, and the mixture was extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=80:20-20:80) to give the title compound (620 mg, yield 99%) as an oil.

¹H NMR (300 MHz, DMSO-d₆) δ 2.04 (3H, s), 2.22 (3H, s), 3.67 (3H, s), 3.88 (3H, s), 6.33 (1H, d, J=2.3 Hz), 6.81-6.86 (1H, m), 7.50-7.67 (3H, m), 8.02 (1H, dd, J=7.6, 1.5 Hz).

### Reference Example 115

### methyl 3-(2,6-dimethyl-1H-benzimidazol-1-yl)-2-methylbenzoate

In the same manner as in Reference Examples 107 and 114, the title compound was synthesized.

¹H NMR (300 MHz, DMSO-d₆) δ 2.01 (3H, s), 2.25 (3H, s), 2.33 (3H, s), 3.88 (3H, s), 6.65 (1H, s), 7.03 (1H, d, J=8.3 Hz), 7.49-7.66 (3H, m), 8.01 (1H, dd, J=7.6, 1.5 Hz).

### Reference Example 116

### methyl 3-[5-methoxy-2-(trifluoromethyl)-1H-benzimidazol-1-yl]-2-methylbenzoate

Methyl 3-[(2-amino-4-methoxyphenyl)amino]-2-methylbenzoate (1.15 g, 4.0 mmol) was suspended in trifluoroacetic acid (10 mL), trifluoroacetic anhydride (2 mL) was added, and the mixture was stirred at 60° C. for 2 hr. The volatile component was evaporated under reduced pressure, an aqueous sodium bicarbonate solution was added, and the mixture was extracted with ethyl acetate. The extract was washed with saturated brine, dried over anhydrous magnesium sulfate, and the volatile component was evaporated under reduced pressure to give the title compound (1.5 g, yield—100%) as an oil.

¹H NMR (300 MHz, DMSO-d₆) δ 2.00 (3H, s), 3.85 (3H, s), 3.88 (3H, s), 6.98 (1H, d, J=9.1 Hz), 7.08 (1H, dd, J=9.1, 2.7 Hz), 7.46 (1H, d, J=2.3 Hz), 7.56-7.63 (1H, m), 7.78 (1H, d, J=7.6 Hz), 8.06 (1H, d, J=8.0 Hz).

### Reference Example 117

### methyl 2-methyl-3-(2-thioxo-2,3-dihydro-1H-benzimidazol-1-yl)benzoate

Methyl 3-[(2-aminophenyl)amino]-2-methylbenzoate (1.02 g, 4.0 mmol) and di-1H-imidazol-1-ylmethanethion (1.07 g, 6.0 mmol) were mixed in tetrahydrofuran (50 mL), and the mixture was stirred at room temperature for 1 hr. An aqueous sodium bicarbonate solution was added, and the mixture was extracted with ethyl acetate. The extract was washed with saturated brine, dried over anhydrous magnesium sulfate, and the volatile component was evaporated under reduced pressure. The obtained residue was purified by silica gel column chromatography (hexane:ethyl acetate=90:10-60:40) to give the title compound (1.2 g, yield—100%) as an oil.

### Reference Example 118

### methyl 2-methyl-3-[2-(methylsulfanyl)-1H-benzimidazol-1-yl]benzoate

Methyl 2-methyl-3-(2-thioxo-2,3-dihydro-1H-benzimidazol-1-yl)benzoate (298 mg, 1 mmol), potassium carbonate (276 mg, 2 mmol) and methyl iodide (284 mg, 2 mmol) were suspended in N,N-dimethylformamide (10 mL), and the mixture was stirred at 60° C. for 1 hr. The reaction mixture was allowed to cool, water was added, and the mixture was extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=90:10-40:60) to give the title compound (290 mg, yield 69%).

¹H NMR (300 MHz, DMSO-d₆) δ 2.03 (3H, s), 2.69 (3H, s), 3.88 (3H, s), 6.88 (1H, d, J=8.0 Hz), 7.11-7.28 (2H, m), 7.53-7.69 (3H, m), 8.02 (1H, dd, J=7.6, 1.5 Hz).

### Reference Example 119

### methyl 3-{2-[(4-chlorobenzyl)sulfanyl]-1H-benzimidazol-1-yl}-2-methylbenzoate

In the same manner as in Reference Example 118, the title compound (yield—100%) was synthesized using 1-(bromomethyl)-4-chlorobenzene as alkyl halide.

¹H NMR (300 MHz, DMSO-d₆) δ 1.93 (3H, s), 3.86 (3H, s), 4.51-4.66 (2H, m), 6.89 (1H, d, J=7.6 Hz), 7.13-7.20 (1H, m), 7.22-7.29 (1H, m), 7.32-7.39 (2H, m), 7.43-7.50 (2H, m), 7.51-7.63 (2H, m), 7.70 (1H, d, J=7.6 Hz), 8.00 (1H, dd, J=7.6, 1.9 Hz).

### Reference Example 120

### methyl 2-methyl-3-[(2-{[(2S)-tetrahydrofuran-2-ylcarbonyl]amino}phenyl)amino]benzoate

Methyl 3-[(2-aminophenyl)amino]-2-methylbenzoate (513 mg, 2.0 mmol), (2S)-tetrahydrofuran-2-carboxylic acid (255 mg, 2.2 mmol), N-(3-dimethylaminopropyl)-N′-ethylcarbodiimide hydrochloride (422 mg, 2.2 mmol) and 1-hydroxybenzotriazole (367 mg, 2.4 mmol) were mixed in N,N-dimethylformamide (10 mL), and the mixture was stirred overnight at room temperature. Water was added, and the mixture was extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=80:20-20:80) to give the title compound (610 mg, yield 69%).

¹H NMR (300 MHz, DMSO-d₆) δ 1.40-1.56 (1H, m), 1.64-1.87 (2H, m), 2.04-2.18 (1H, m), 2.37 (3H, s), 3.36-3.45 (1H, m), 3.56-3.68 (1H, m), 3.83 (3H, s), 4.27-4.36 (1H, m), 6.57 (1H, d, J=6.8 Hz), 7.03-7.22 (6H, m), 7.89-7.98 (1H, m), 9.08 (1H, s).

### Reference Example 121

### methyl 2-methyl-3-(7-methyl-1H-benzimidazol-1-yl)benzoate

Methyl 3-[(2-amino-6-methylphenyl)amino]-2-methylbenzoate (270 mg, 1 mmol) and trimethoxymethane (313 mg, 3 mmol) were mixed in acetic acid (10 mL), and the mixture was stirred at 60° C. for 3 hr. The volatile component was evaporated under reduced pressure, an aqueous sodium bicarbonate solution was added, and the mixture was extracted with ethyl acetate. The extract was dried over anhydrous magnesium sulfate, and the volatile component was evaporated under reduced pressure. The obtained residue was purified by silica gel column chromatography (hexane:ethyl acetate=90:10-60:40) to give the title compound (310 mg, yield—100%).

¹H NMR (300 MHz, DMSO-d₆) δ 1.86 (3H, s), 2.05 (3H, s), 3.88 (3H, s), 7.01 (1H, d, J=7.2 Hz), 7.17 (1H, t, J=7.8 Hz), 7.49-7.56 (1H, m), 7.61 (1H, d, J=8.3 Hz), 7.73 (1H, d, J=6.4 Hz), 7.96-8.01 (1H, m), 8.23 (1H, s).

### Reference Example 122

### methyl 3-(2-ethyl-1H-benzimidazol-1-yl)-2-methylbenzoate

Methyl 3-[(2-aminophenyl)amino]-2-methylbenzoate (541 mg, 2.0 mmol) was dissolved in N,N-dimethylacetamide (10 mL), propanoyl chloride (222 mg, 2.4 mmol) was added, and the mixture was stirred at room temperature for 30 min. The mixture was diluted with aqueous sodium bicarbonate solution and extracted with ethyl acetate. The extract was washed with saturated brine, dried over anhydrous magnesium sulfate, and the volatile component was evaporated under reduced pressure. The residue was dissolved in acetic acid (20 mL), and the obtained solution was stirred overnight at 110° C. The mixture was allowed to cool, the volatile component was evaporated, and the residue was diluted with aqueous sodium bicarbonate solution, extracted with ethyl acetate and the extract was dried over anhydrous magnesium sulfate. The volatile component was evaporated under reduced pressure to give the title compound (550 mg, yield 94%) as a solid.

¹H NMR (300 MHz, DMSO-d₆) δ 1.18-1.25 (3H, m), 1.98-2.01 (3H, m), 2.53-2.63 (2H, m), 3.88 (3H, s), 6.86 (1H, d, J=7.3 Hz), 7.13-7.27 (2H, m), 7.54-7.61 (1H, m), 7.64-7.71 (2H, m), 8.02 (1H, dd, J=7.7, 1.5 Hz).

### Reference Example 123

### methyl 3-[2-(methoxymethyl)-1H-benzimidazol-1-yl]-2-methylbenzoate

Methyl 3-[(2-aminophenyl)amino]-2-methylbenzoate (540 mg, 2.0 mmol), methoxyacetic acid (180 mg, 2.2 mmol), N-(3-dimethylaminopropyl)-N′-ethylcarbodiimide hydrochloride (422 mg, 2.2 mmol) and 1-hydroxybenzotriazole (367 mg, 2.4 mmol) were suspended in N,N-dimethylformamide (10 mL), and the suspension was stirred overnight at room temperature. The mixture was diluted with aqueous sodium bicarbonate solution and extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=90:10-60:40) to give an oil (380 mg). The obtained compound was stirred overnight in acetic acid (10 mL) at 110° C. The volatile component was evaporated under reduced pressure. The residue was diluted with aqueous sodium bicarbonate solution, and the mixture was extracted with ethyl acetate. The extract was dried over anhydrous magnesium sulfate and the volatile component was evaporated under reduced pressure to give the title compound (250 mg, yield 89%) as an oil.

¹H NMR (300 MHz, DMSO-d₆) δ 2.00 (3H, s), 3.14 (3H, s), 3.88 (3H, s), 4.35-4.42 (1H, m), 4.46-4.52 (1H, m), 6.90-6.97 (1H, m), 7.21-7.33 (2H, m), 7.51-7.60 (1H, m), 7.63-7.68 (1H, m), 7.74-7.79 (1H, m), 7.98-8.04 (1H, m).

### Reference Example 124

### methyl 3-(6-chloro-2-ethyl-1H-benzimidazol-1-yl)-2-methylbenzoate

N-(2-Bromo-4-chlorophenyl)propanamide (788 mg, 3.0 mmol), methyl 3-amino-2-methylbenzoate (496 mg, 3.0 mmol), tris(dibenzylideneacetone)dipalladium (110 mg, 0.12 mmol), 2,2′-bis(diphenylphosphino)diphenyl ether (129 mg, 0.24 mmol) and tripotassium phosphate (1.91 g, 9.0 mmol) were suspended in toluene (30 mL), and the mixture was stirred under an argon atmosphere at 110° C. for 5 days. The reaction mixture was allowed to cool, diluted with saturated brine, and the mixture was extracted with ethyl acetate. The extract was dried over anhydrous magnesium sulfate, and the volatile component was evaporated under reduced pressure. The residue was purified by silica gel column chromatography (hexane:ethyl acetate=90:10-60:40). The obtained solid was dissolved in acetic acid (10 mL) and the mixture was stirred at 110° C. overnight. The volatile component was evaporated under reduced pressure. The residue was purified by silica gel column chromatography (hexane:ethyl acetate=90:10-40:60) to give the title compound as an oil (360 mg, yield 36%).

¹H NMR (300 MHz, DMSO-d₆) δ 1.18-1.26 (3H, m), 2.01 (3H, s), 2.52-2.60 (2H, m), 3.88 (3H, s), 6.93 (1H, d, J=1.9 Hz), 7.26 (1H, dd, J=8.5, 2.1 Hz), 7.55-7.75 (3H, m), 8.02 (1H, s).

### Reference Example 125

### methyl 3-(2-ethyl-6-fluoro-1H-benzimidazol-1-yl)-2-methylbenzoate

In the same manner as in Reference Example 122, the title compound was synthesized using methyl 3-[(2-amino-5-fluorophenyl)amino]-2-methylbenzoate.

¹H NMR (300 MHz, CDCl₃) δ 1.32 (3H, t, J=7.6 Hz), 2.15 (3H, s), 2.62 (2H, q, J=7.6 Hz), 3.96 (3H, s), 6.56 (1H, dd, J=8.5, 2.5 Hz), 6.96-7.06 (1H, m), 7.37-7.52 (2H, m), 7.71 (1H, dd, J=8.7, 4.5 Hz), 8.07 (1H, dd, J=7.8, 1.7 Hz). MS m/z 313 (M+H)⁺.

### Reference Example 126

### [3-(2-ethyl-6-fluoro-1H-benzimidazol-1-yl)-2-methylphenyl]methanol

To a solution of methyl 3-(2-ethyl-6-fluoro-1H-benzimidazol-1-yl)-2-methylbenzoate (10 g, 32 mmol) in tetrahydrofuran (300 mL) was added lithium aluminum hydride (2.43 g, 64 mmol), and the mixture was stirred at room temperature for 30 min. Sodium sulfate 10 hydrate was added, and then ethyl acetate was added, and the mixture was filtered through a celite pad. The filtrate was concentrated and the residue was purified by silica gel column chromatography (hexane:ethyl acetate=70:30-10:90) to give the title compound (7.9 g, yield 87%) as an oil.

¹H NMR (300 MHz, CDCl₃) δ 1.32 (3H, t, J=7.5 Hz), 1.92 (3H, s), 2.63 (2H, q, J=7.5 Hz), 4.83 (2H, d, J=5.7 Hz), 6.57 (1H, dd, J=8.7, 2.3 Hz), 6.93-7.24 (2H, m), 7.41 (1H, t, J=7.7 Hz), 7.59-7.74 (2H, m).

MS m/z 285 (M+H)⁺.

### Reference Example 127

### 3-(2-ethyl-6-fluoro-1H-benzimidazol-1-yl)-2-methylbenzaldehyde

To a solution of [3-(2-ethyl-6-fluoro-1H-benzimidazol-1-yl)-2-methylphenyl]methanol (6.8 g, 23.4 mmol) in acetonitrile (200 mL) was added Dess-Martin reagent (12.2 g, 28.7 mmol) at 0° C., and the mixture was stirred at room temperature for 30 min. An aqueous sodium thiosulfate solution was added, and the mixture was extracted with ethyl acetate. The extract was washed with saturated brine, dried over anhydrous magnesium sulfate, and the volatile component was evaporated under reduced pressure. The obtained residue was purified by silica gel column chromatography (hexane:ethyl acetate=70:30-20:80) to give the title compound (5.5 g, yield 83%) as an oil.

¹H NMR (300 MHz, CDCl₃) δ 1.33 (3H, t, J=7.5 Hz), 2.27 (3H, s), 2.62 (2H, q, J=7.5 Hz), 6.55 (1H, dd, J=8.3, 2.3 Hz), 6.98-7.07 (1H, m), 7.47-7.54 (1H, m), 7.57-7.65 (1H, m), 7.73 (1H, dd, J=8.9, 4.7 Hz), 8.05 (1H, dd, J=7.5, 1.5 Hz), 10.38 (1H, s).

MS m/z 283 (M+H)⁺.

### Reference Example 128

### methyl {(3S)-6-[(3-bromo-2-methylbenzyl)(trifluoroacetyl)amino]-2,3-dihydro-1-benzofuran-3-yl}acetate

Methyl {(3S)-6-[(3-bromo-2-methylbenzyl)amino]-2,3-dihydro-1-benzofuran-3-yl}acetate (13.2 g, 33.8 mmol) and triethylamine (6.8 g, 67.6 mmol) were dissolved in tetrahydrofuran (150 mL), trifluoroacetic anhydride (852 mg, 40.6 mmol) was added at 0° C. and the mixture was stirred for 1 hr. The reaction mixture was diluted with aqueous sodium bicarbonate solution and extracted with ethyl acetate. The extract was washed with saturated brine, dried over anhydrous magnesium sulfate, and the volatile component was evaporated under reduced pressure. The obtained residue was purified by silica gel column chromatography (hexane:ethyl acetate=95:5-40:60) to give the title compound (13.5 g, yield 82%) as an oil.

¹H NMR (300 MHz, DMSO-d₆) δ 2.20 (3H, s), 2.57-2.68 (1H, m), 2.76-2.87 (1H, m), 3.61 (3H, s), 3.70-3.89 (1H, m), 4.21-4.33 (1H, m), 4.73 (1H, t, J=9.2 Hz), 5.00 (2H, s), 6.64 (1H, d, J=7.9 Hz), 6.75 (1H, s), 7.00-7.12 (2H, m), 7.18 (1H, d, J=7.9 Hz), 7.52 (1H, dd, J=7.2, 1.9 Hz).

### Reference Example 129

### methyl {(3S)-6-[(3-{[2-amino-3-(benzyloxy)phenyl]amino}-2-methylbenzyl)(trifluoroacetyl)amino]-2,3-dihydro-1-benzofuran-3-yl}acetate

Methyl {(3S)-6-[(3-bromo-2-methylbenzyl)(trifluoroacetyl)amino]-2,3-dihydro-1-benzofuran-3-yl}acetate (4.08 g, 8.41 mmol), 3-(benzyloxy)-2-nitroaniline (1.87 g, 7.65 mmol), tris(dibenzylideneacetone)dipalladium (347 mg, 0.38 mmol), 2-(dicyclohexylphosphino)-2′,4′,6′-triisopropyl-1′,1′-biphenyl (367 mg, 0.77 mmol) and tripotassium phosphate (3.25 g, 15.3 mmol) were suspended in toluene (40 mL), and the mixture was stirred under an argon atmosphere overnight at 100° C. The reaction mixture was allowed to cool, diluted with water and extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=90:10-60:40) to give an oil (4.04 g, yield 80%). The obtained oil was suspended in ethanol (30 mL)-water (5 mL)-acetic acid (5 mL), reduced iron (3.43 g, 61.5 mmol) and calcium chloride (6.82 g, 61.5 mmol) were added and the mixture was stirred at 70° C. for 2 hr. The volatile component was evaporated under reduced pressure, and the residue was extracted with ethyl acetate, and the extract was washed with aqueous sodium bicarbonate solution and saturated brine. The organic layer was dried over anhydrous magnesium sulfate, and the volatile component was evaporated under reduced pressure. The obtained residue was purified by silica gel column chromatography to give the title compound (2.5 g, yield 53%) as an oil.

### Reference Example 130

### methyl [(3S)-6-{[3-(2-ethoxy-4-hydroxy-1H-benzimidazol-1-yl)-2-methylbenzyl](trifluoroacetyl)amino}-2,3-dihydro-1-benzofuran-3-yl]acetate

Methyl {(3S)-6-[(3-{[2-amino-3-(benzyloxy)phenyl]amino}-2-methylbenzyl)(trifluoroacetyl)amino]-2,3-dihydro-1-benzofuran-3-yl}acetate (1.23 g, 2.0 mmol) and tetraethoxymethane (883 mg, 6.0 mmol) were dissolved in acetic acid (10 mL), and the mixture was stirred at 60° C. for 2 hr. The volatile component was evaporated under reduced pressure, and the residue was diluted with an aqueous sodium bicarbonate solution, and the mixture was extracted with ethyl acetate. The extract was washed with saturated brine, and the organic layer was dried over anhydrous magnesium sulfate. The volatile component was evaporated under reduced pressure. The obtained residue was suspended in methanol (30 mL), palladium-carbon (300 mg) was added, and the mixture was stirred under a hydrogen atmosphere overnight. The catalyst was removed, and the filtrate was evaporated under reduced pressure. The obtained residue was purified by silica gel column chromatography (hexane:ethyl acetate=80:20-20:80) to give the title compound (810 mg, yield 66%) as a solid.

### Reference Example 131

### methyl [(3S)-6-{[3-(2-ethyl-4-hydroxy-1H-benzimidazol-1-yl)-2-methylbenzyl](trifluoroacetyl)amino}-2,3-dihydro-1-benzofuran-3-yl]acetate

Methyl {(3S)-6-[(3-{[2-amino-3-(benzyloxy)phenyl]amino}-2-methylbenzyl)(trifluoroacetyl)amino]-2,3-dihydro-1-benzofuran-3-yl}acetate (1.23 g, 2.0 mmol) was dissolved in N,N-dimethylacetamide (20 mL), propanoyl chloride (222 mg, 2.4 mmol) was added, and the mixture was stirred at room temperature for 1 hr. The volatile component was evaporated under reduced pressure, and the residue was diluted with an aqueous sodium bicarbonate solution, and the mixture was extracted with ethyl acetate. The extract was washed with saturated brine, the organic layer was dried over anhydrous magnesium sulfate, and the volatile component was evaporated under reduced pressure. To the obtained residue was added acetic acid (20 mL) and the mixture was stirred at 90° C. overnight. The volatile component was evaporated under reduced pressure, the residue was diluted with an aqueous sodium bicarbonate solution, and the mixture was extracted with ethyl acetate. The extract was washed with saturated brine, the organic layer was dried over anhydrous magnesium sulfate, and the volatile component was evaporated under reduced pressure. The obtained residue was suspended in methanol (40 mL), 10% palladium-carbon (50% water-containing product, 200 mg) was added, and the mixture was stirred under a hydrogen atmosphere for 3 hr. The catalyst was removed, and the filtrate was evaporated under reduced pressure. The obtained residue was purified by silica gel column chromatography (hexane:ethyl acetate=50:50-20:80) to give the title compound (880 mg, yield 78%) as an oil.

¹H NMR (300 MHz, DMSO-d₆) δ 1.11-1.21 (3H, m), 1.49-1.55 (3H, m), 2.41-2.48 (2H, m), 2.55-2.66 (1H, m), 2.76-2.87 (1H, m), 3.60 (3H, s), 3.73-3.86 (1H, m), 4.22-4.31 (1H, m), 4.69-4.79 (1H, m), 5.07 (2H, s), 6.13 (1H, d, J=7.9 Hz), 6.56 (1H, d, J=7.9 Hz), 6.64-6.71 (2H, m), 6.92 (1H, td, J=7.9, 1.5 Hz), 7.20-7.42 (4H, m), 9.75 (1H, s).

### Reference Example 132

### 3-bromo-2,4-dimethylbenzaldehyde

To a solution of 2-bromo-1,3-dimethylbenzene (5.00 g, 27.0 mmol) in dichloromethane (5 mL) was slowly added dropwise a solution of dichloro(methoxy)methane (2.69 mL, 29.7 mmol) and titanium tetrachloride (5.96 mL, 54.0 mmol) in dichloromethane (20 mL) at −78° C., the reaction mixture was heated to room temperature and stirred for 4 hr. The reaction mixture was poured into water, and the mixture was extracted with ethyl acetate. The extract was dried over sodium sulfate, and concentrated under reduced pressure. The residue was purified by silica gel column chromatography (hexane:ethyl acetate=100:0-85:15) to give the title compound (5.03 g, yield 87%) as a colorless oil.

¹H NMR (300 MHz, CDCl₃) δ 2.50 (3H, s), 2.78 (3H, s), 7.26 (1H, d, J=8.0 Hz), 7.67 (1H, d, J=7.6 Hz), 10.22 (1H, s).

### Reference Example 133

### 1-(difluoromethyl)-3,5-dimethyl-4-(4,4,5,5-tetramethyl-1,3,2-dioxaborolan-2-yl)-1H-pyrazole

A mixed solution of 3,5-dimethyl-4-(4,4,5,5-tetramethyl-1,3,2-dioxaborolan-2-yl)-1H-pyrazole (0.314 g, 1.41 mmol), sodium chlorodifluoroacetate (0.259 g, 1.70 mmol) and 18-crown-6 (74.7 mg, 0.283 mmol) in acetonitrile (8 mL) was heated under reflux for 2 hr. The reaction mixture was diluted with ammonium chloride solution, and the mixture was extracted with ethyl acetate. The extract was dried over sodium sulfate, and concentrated under reduced pressure to give the title compound (0.428 g, yield 100%) as a beige solid.

¹H NMR (300 MHz, CDCl₃) δ 1.30 (12H, s), 2.33 (3H, s), 2.57 (3H, s), 7.13 (1H, t, J=59.1 Hz).

MS m/z 273 (M+H)⁺.

### Reference Example 134

### 3-(1,4-dioxaspiro[4.5]dec-7-en-8-yl)-2,4-dimethylbenzaldehyde

In the same manner as in Example 184, the title compound was obtained as a yellow oil from 3-bromo-2,4-dimethylbenzaldehyde and 8-(4,4,5,5-tetramethyl-1,3,2-dioxaborolan-2-yl)-1,4-dioxaspiro[4.5]dec-7-ene. yield 86%.

¹H NMR (300 MHz, CDCl₃) δ 1.92 (2H, t, J=6.4 Hz), 2.24-2.37 (5H, m), 2.41-2.48 (2H, m), 2.56 (3H, s), 4.01-4.07 (4H, m), 5.34-5.41 (1H, m), 7.18 (1H, d, J=7.9 Hz), 7.62 (1H, d, J=7.9 Hz), 10.26 (1H, s).

### Reference Example 135

### 3-(1,4-dioxa-8-azaspiro[4.5]dec-8-yl)-2,4-dimethylbenzaldehyde

A solution of 3-bromo-2,4-dimethylbenzaldehyde (0.919 g, 4.31 mmol), 1,4-dioxa-8-azaspiro[4.5]decane (0.865 g, 6.04 mmol) and cesium carbonate (4.22 g, 12.9 mmol) in toluene (7 mL) was substituted with argon, and tris(dibenzylideneacetone)dipalladium (0) (0.158 g, 0.173 mmol) and 2,2′-bis(diphenylphosphino)-1,1′-binaphthyl (0.215 g, 0.345 mmol) were added. The reaction mixture was stirred under an argon atmosphere at 110° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=100:0-85:15) to give the title compound (0.128 g, yield 11%) as a yellow oil.

¹H NMR (300 MHz, CDCl₃) δ 1.80-1.89 (4H, m), 2.39 (3H, s), 2.63 (3H, s), 3.08-3.26 (4H, m), 4.02 (4H, s), 7.13 (1H, d, J=7.9 Hz), 7.52 (1H, d, J=7.7 Hz), 10.23 (1H, s).

MS m/z 276 (M+H)⁺.

### Reference Example 136

### methyl 2-methyl-3-[2-(trifluoromethyl)-1H-benzimidazol-1-yl]benzoate

A mixed solution of methyl 3-[(2-aminophenyl)amino]-2-methylbenzoate (1.13 g, 4.40 mmol) in trifluoroacetic acid (10 mL) and trifluoroacetic anhydride (2 mL) was stirred with heating under reflux for 16 hr, and concentrated under reduced pressure. To the residue was added saturated aqueous sodium hydrogen carbonate, and the mixture was extracted with ethyl acetate. The extract was dried over sodium sulfate, and concentrated under reduced pressure. The residue was purified by silica gel column chromatography (hexane:ethyl acetate=90:10-50:50) to give the title compound (1.44 g, yield 98%) as a colorless oil.

¹H NMR (300 MHz, CDCl₃) δ 2.13 (3H, s), 3.95 (3H, s), 6.95-7.00 (1H, m), 7.34-7.52 (4H, m), 7.94-8.00 (1H, m), 8.09-8.16 (1H, m).

MS m/z 335 (M+H)⁺.

### Reference Example 137

### 2-methyl-3-[2-(trifluoromethyl)-1H-benzimidazol-1-yl]benzaldehyde

To a solution of methyl 2-methyl-3-[2-(trifluoromethyl)-1H-benzimidazol-1-yl]benzoate (1.44 g, 4.31 mmol) in tetrahydrofuran (15 mL) was added dropwise a solution of diisobutylaluminum hydride in toluene (1.6 M, 6.74 mL, 10.8 mmol) at 0° C. The reaction mixture was stirred at the same temperature for 1.5 hr, sodium sulfate 10 hydrate (3.48 g, 10.8 mmol) was slowly added, and the mixture was further stirred at room temperature for 1.5 hr. Insoluble material was filtered off, and the filtrate was concentrated under reduced pressure to give {2-methyl-3-[2-(trifluoromethyl)-1H-benzimidazol-1-yl]phenyl}methanol as a colorless crude oil (1.51 g). To a solution of {2-methyl-3-[2-(trifluoromethyl)-1H-benzimidazol-1-yl]phenyl}methanol (0.444 g, 1.45 mmol) obtained above in acetonitrile (7 mL) was slowly added at 0° C. a Dess-Martin reagent (0.738 g, 1.74 mmol). The reaction mixture was stirred at room temperature for 40 min, aqueous sodium bicarbonate solution and saturated aqueous sodium thiosulfate solution were added, and the mixture was further stirred for min. The reaction mixture was extracted with ethyl acetate, and the obtained extract was dried over sodium sulfate, and concentrated under reduced pressure to give the title compound (0.296 g, yield 67%) as a yellow oil.

¹H NMR (300 MHz, CDCl₃) δ 2.24 (3H, s), 6.94-7.02 (1H, m), 7.36-7.50 (2H, m), 7.54-7.67 (2H, m), 7.99 (1H, dd, J=8.2, 1.2 Hz), 8.09 (1H, dd, J=7.2, 2.3 Hz), 10.39 (1H, s).

MS m/z 305 (M+H)⁺.

### Reference Example 138

### methyl 3-(2-cyclopropyl-1H-benzimidazol-1-yl)-2-methylbenzoate

In the same manner as in Reference Example 122, the title compound was obtained as a purple oil from methyl 3-[(2-aminophenyl)amino]-2-methylbenzoate and cyclopropanecarbonyl chloride. yield 95%.

¹H NMR (300 MHz, CDCl₃) δ 0.94-1.05 (2H, m), 1.25-1.35 (2H, m), 1.52-1.64 (1H, m), 2.22 (3H, s), 3.96 (3H, s), 6.87 (1H, d, J=8.0 Hz), 7.11-7.29 (2H, m), 7.42-7.53 (2H, m), 7.72 (1H, d, J=7.6 Hz), 8.06 (1H, dd, J=7.2, 1.9 Hz).

MS m/z 307 (M+H)⁺.

### Reference Example 139

### 3-(2-cyclopropyl-1H-benzimidazol-1-yl)-2-methylbenzaldehyde

In the same manner as in Reference Example 137, the title compound was obtained as a yellow oil from methyl 3-(2-cyclopropyl-1H-benzimidazol-1-yl)-2-methylbenzoate. yield 81% (2 steps).

¹H NMR (300 MHz, CDCl₃) δ 0.95-1.06 (2H, m), 1.27-1.37 (2H, m), 1.50-1.62 (1H, m), 2.34 (3H, s), 6.87 (1H, d, J=7.6 Hz), 7.12-7.21 (1H, m), 7.22-7.31 (1H, m), 7.56-7.64 (2H, m), 7.74 (1H, d, J=8.0 Hz), 8.00-8.09 (1H, m), 10.41 (1H, s).

MS m/z 277 (M+H)⁺.

### Reference Example 140

### methyl [(3S)-6-{[2-formyl-3-hydroxy-4-(trifluoromethyl)benzyl](trifluoroacetyl)amino}-2,3-dihydro-1-benzofuran-3-yl]acetate

2-(Dimethoxymethyl)-3-(methoxymethoxy)-4-(trifluoromethyl)benzaldehyde (1.14 g, 3.69 mmol) synthesized according to WO2006/046593 and methyl [(3S)-6-amino-2,3-dihydro-1-benzofuran-3-yl]acetate (0.765 g, 3.69 mmol) were dissolved in acetic acid (0.634 mL, 11.1 mmol) and acetonitrile (16 mL), sodium triacetoxyborohydride (1.57 g, 7.39 mmol) was added at 0° C., and the mixture was stirred at 0° C. for 1 hr. The reaction mixture was neutralized with saturated aqueous sodium hydrogen carbonate, and the mixture was extracted with ethyl acetate. The extract was dried over sodium sulfate, and concentrated under reduced pressure. The residue was purified by silica gel column chromatography (hexane:ethyl acetate=100:0-67:33) to give methyl [(3S)-6-{[2-(dimethoxymethyl)-3-(methoxymethoxy)-4-(trifluoromethyl)benzyl]amino}-2,3-dihydro-1-benzofuran-3-yl]acetate (1.51 g) as a yellow oil. To a solution of methyl [(3S)-6-{[2-(dimethoxymethyl)-3-(methoxymethoxy)-4-(trifluoromethyl)benzyl]amino}-2,3-dihydro-1-benzofuran-3-yl]acetate (1.51 g, 3.01 mmol) obtained above in methylene chloride (0.10 mL) were successively added triethylamine (0.630 mL, 4.52 mmol) and trifluoroacetic anhydride (0.511 mL, 3.62 mmol) at 0° C. The reaction mixture was warmed to room temperature, and stirred for 4 hr. The reaction mixture was poured into saturated aqueous sodium hydrogen carbonate, and the mixture was extracted with ethyl acetate. The extract was washed with saturated aqueous sodium hydrogen carbonate and saturated brine, dried over sodium sulfate, and concentrated under reduced pressure to give a crude product (1.83 g) of methyl [(3S)-6-{[2-(dimethoxymethyl)-3-(methoxymethoxy)-4-(trifluoromethyl)benzyl](trifluoroacetyl)amino}-2,3-dihydro-1-benzofuran-3-yl]acetate as a yellow oil. To a solution of methyl [(3S)-6-{[2-(dimethoxymethyl)-3-(methoxymethoxy)-4-(trifluoromethyl)benzyl](trifluoroacetyl)amino}-2,3-dihydro-1-benzofuran-3-yl]acetate (1.83 g, 3.07 mmol) obtained above in acetone (10 mL) was added p-toluenesulfonic acid monohydrate (0.817 g, 4.29 mmol), and the mixture was stirred at room temperature for 16 hr. The reaction mixture was poured into a mixture of sodium bicarbonate aqueous solution and brine, and the mixture was extracted with ethyl acetate. The extract was washed with saturated brine, dried over anhydrous sodium sulfate, and concentrated under reduced pressure. The residue was purified by silica gel column chromatography (hexane:ethyl acetate=95:5-60:40) to give the title compound (1.53 g, yield 81% (3 steps)) as an orange oil.

¹H NMR (300 MHz, CDCl₃) δ 2.62 (1H, dd, J=16.8, 9.0 Hz), 2.78 (1H, dd, J=16.6, 5.7 Hz), 3.73 (3H, s), 3.82-3.98 (1H, m), 4.33 (1H, dd, J=9.3, 6.5 Hz), 4.83 (1H, t, J=9.2 Hz), 5.23 (2H, br s), 6.44 (1H, dd, J=7.7, 1.1 Hz), 6.52 (1H, d, J=1.7 Hz), 6.88 (1H, d, J=7.9 Hz), 7.12 (1H, dd, J=7.8, 0.8 Hz), 7.73 (1H, d, J=8.1 Hz), 9.97 (1H, s), 12.70 (1H, s).

MS m/z 504 (M−H)⁻.

### Reference Example 141

### methyl [(3S)-6-{[3-hydroxy-2-methyl-4-(trifluoromethyl)benzyl](trifluoroacetyl)amino}-2,3-dihydro-1-benzofuran-3-yl]acetate

Methyl [(3S)-6-{[2-formyl-3-hydroxy-4-(trifluoromethyl)benzyl](trifluoroacetyl)amino}-2,3-dihydro-1-benzofuran-3-yl]acetate (1.53 g, 3.03 mmol) was dissolved in methanol (10 mL), 10% palladium-carbon (50% water-containing product, 1.5 g) was added, and the mixture was stirred under a hydrogen atmosphere (balloon pressure) at room temperature for 16 hr. The catalyst was filtered off, and the filtrate was concentrated under reduced pressure. The residue was dissolved in ethyl acetate, dried over sodium sulfate, and concentrated under reduced pressure to give the title compound (1.36 g, yield 91%) as a pale-yellow oil.

¹H NMR (300 MHz, CDCl₃) δ 1.26 (1H, t, J=7.2 Hz), 2.05 (3H, s), 2.59 (1H, dd, J=16.3, 8.7 Hz), 2.76 (1H, dd, J=16.7, 5.7 Hz), 3.71 (3H, s), 3.81-3.92 (1H, m), 4.30 (1H, dd, J=9.1, 6.4 Hz), 4.79 (1H, t, J=9.3 Hz), 4.96 (2H, s), 6.41-6.50 (2H, m), 6.79 (1H, d, J=8.0 Hz), 7.06 (1H, d, J=8.7 Hz), 7.16-7.28 (1H, m).

### Reference Example 142

### methyl [(3S)-6-{[2-methyl-4-(trifluoromethyl)-3-{[(trifluoromethyl)sulfonyl]oxy}benzyl](trifluoroacetyl)amino}-2,3-dihydro-1-benzofuran-3-yl]acetate

To a solution of methyl [(3S)-6-{[3-hydroxy-2-methyl-4-(trifluoromethyl)benzyl](trifluoroacetyl)amino}-2,3-dihydro-1-benzofuran-3-yl]acetate (1.36 g, 2.77 mmol) in pyridine (8 mL) was added dropwise trifluoromethanesulfonic anhydride (1.17 mL, 6.91 mmol) at 0° C. The reaction mixture was stirred at room temperature for 4 hr, and concentrated under reduced pressure. To the residue was added 1 M aqueous hydrochloric acid solution, and the mixture was extracted with ethyl acetate. The extract was washed with 1 M aqueous hydrochloric acid solution and saturated brine, dried over anhydrous sodium sulfate, and concentrated under reduced pressure. The residue was purified by silica gel column chromatography (hexane:ethyl acetate=95:5-65:35) to give the title compound (1.53 g, yield 89%) as a pale-yellow oil.

¹H NMR (300 MHz, CDCl₃) δ 2.19 (3H, s), 2.59 (1H, dd, J=16.7, 9.1 Hz), 2.76 (1H, dd, J=16.7, 5.7 Hz), 3.71 (3H, s), 3.80-3.96 (1H, m), 4.31 (1H, dd, J=9.3, 6.6 Hz), 4.81 (1H, t, J=9.3 Hz), 5.02 (2H, br s), 6.40-6.48 (1H, m), 6.51 (1H, d, J=1.5 Hz), 7.08 (1H, dd, J=8.0, 0.8 Hz), 7.36 (1H, d, J=8.0 Hz), 7.55 (1H, d, J=8.0 Hz).

MS m/z 622 (M−H)⁻.

### Reference Example 143

### methyl [(3S)-6-{[2=methyl-3-(6-morpholin-4-ylpyridin-3-yl)-4-(trifluoromethyl)benzyl](trifluoroacetyl)amino}-2,3-dihydro-1-benzofuran-3-yl]acetate

In the same manner as in Example 184, the title compound was obtained as a yellow oil from methyl [(3S)-6-{[2-methyl-4-(trifluoromethyl)-3-{[(trifluoromethyl)sulfonyl]oxy}benzyl](trifluoroacetyl)amino}-2,3-dihydro-1-benzofuran-3-yl]acetate and (6-morpholin-4-ylpyridin-3-yl)boronic acid. yield 100%.

¹H NMR (300 MHz, CDCl₃) δ 1.82 (3H, s), 2.53-2.66 (1H, m), 2.71-2.82 (1H, m), 3.52-3.60 (4H, m), 3.72 (3H, s), 3.80-3.95 (5H, m), 4.26-4.35 (1H, m), 4.80 (1H, t, J=9.3 Hz), 4.89-5.10 (2H, m), 6.49-6.60 (2H, m), 6.68 (1H, d, J=8.7 Hz), 7.09 (1H, d, J=8.0 Hz), 7.21-7.29 (2H, m), 7.54 (1H, d, J=8.3 Hz), 7.91 (1H, d, J=1.5 Hz).

MS m/z 638 (M+H)⁺.

### Reference Example 144

### methyl [(3S)-6-{[3-(6-methoxypyridin-3-yl)-2-methyl-4-(trifluoromethyl)benzyl](trifluoroacetyl)amino}-2,3-dihydro-1-benzofuran-3-yl]acetate

In the same manner as in Example 184, the title compound was obtained as a colorless oil from methyl [(3S)-6-{[2-methyl-4-(trifluoromethyl)-3-{[(trifluoromethyl)sulfonyl]oxy}benzyl](trifluoroacetyl)amino}-2,3-dihydro-1-benzofuran-3-yl]acetate and (6-methoxypyridin-3-yl)boronic acid. yield 95%.

¹H NMR (300 MHz, CDCl₃) δ 1.79 (3H, s), 2.54-2.65 (1H, m), 2.71-2.82 (1H, m), 3.72 (3H, s), 3.81-3.95 (1H, m), 3.99 (3H, s), 4.26-4.36 (1H, m), 4.76-4.85 (1H, m), 4.91-5.10 (2H, m), 6.49-6.62 (2H, m), 6.80 (1H, d, J=8.3 Hz), 7.10 (1H, d, J=8.0 Hz), 7.24-7.36 (2H, m), 7.56 (1H, d, J=8.3 Hz), 7.89 (1H, d, J=1.9 Hz).

MS m/z 583 (M+H)⁺.

### Reference Example 145

### 3-bromo-2-(trifluoromethyl)imidazo[1,2-a]pyridine

To a solution of 2-(trifluoromethyl)imidazo[1,2-a]pyridine (2.81 g, 15.1 mmol) in ethanol (30 mL) was added dropwise aqueous bromine solution (1.89 M, 8.00 mL, 15.1 mmol), and the mixture was stirred at room temperature for 30 min. The reaction mixture was concentrated under reduced pressure, poured into a mixture of saturated aqueous sodium hydrogen carbonate and saturated aqueous sodium thiosulfate solution, and the mixture was extracted with ethyl acetate. The extract was dried over sodium sulfate, and concentrated under reduced pressure. The obtained solid was washed with hexane and diisopropyl ether to give the title compound (2.90 g, yield 73%) as a beige solid.

H NMR (300 MHz, CDCl₃) δ 7.02-7.11 (1H, m), 7.34-7.43 (1H, m), 7.65-7.74 (1H, m), 8.17-8.24 (1H, m).

### Reference Example 146

### 4-(5-bromo-3-methylpyridin-2-yl)morpholine

A mixed solution of 2,5-dibromo-3-methylpyridine (3.00 g, 12.0 mmol) and morpholine (15 mL) was stirred by microwave at 210° C. for 10 min. The reaction mixture was diluted with water, and the mixture was extracted with ethyl acetate. The extract was washed with water, dried over sodium sulfate, and concentrated under reduced pressure. The residue was purified by silica gel column chromatography (hexane:ethyl acetate=100:0-75:25) to give the title compound (3.13 g, yield 100%) as a white solid.

¹H NMR (300 MHz, CDCl₃) δ 2.26 (3H, s), 3.08-3.17 (4H, m), 3.79-3.88 (4H, m), 7.53 (1H, d, J=3.0 Hz), 8.19 (1H, d, J=2.6 Hz).

### Reference Example 147

### (5-methyl-6-morpholin-4-ylpyridin-3-yl)boronic acid

Under an argon atmosphere, to a suspension of magnesium (0.324 g, 13.4 mmol) in tetrahydrofuran (5 mL) was added dropwise 1,2-dibromoethane (0.2 mL), and the mixture was stirred for 1 min. A solution of 4-(5-bromo-3-methylpyridin-2-yl)morpholine (3.12 g, 12.1 mmol) in tetrahydrofuran (15 mL) was slowly added dropwise at 70° C. After completion of the dropwise addition, the reaction mixture was stirred at 70° C. for min. Then, the reaction mixture was cooled to 0° C., and trimethyl borate (2.03 mL, 18.2 mmol) was slowly added dropwise. The reaction mixture was stirred at room temperature for 1.5 hr, neutralized with 1 M aqueous hydrochloric acid solution, and extracted with ethyl acetate. The extract was dried over sodium sulfate, and concentrated under reduced pressure. The obtained solid was washed with diisopropyl ether-ethyl acetate to give the title compound (2.04 g, yield 76%) as a pale-yellow solid.

MS m/z 223 (M+H)⁺.

### Reference Example 148

### 4-(5-bromopyridin-2-yl)morpholine

A mixture of 2,5-dibromopyridine (20.0 g, 84.4 mmol), morpholine (106 mL) and copper (I) oxide (0.181 g, 1.23 mmol) was heated under reflux under an argon atmosphere for 3 hr. The reaction mixture was concentrated under reduced pressure, 1 M aqueous sodium hydroxide solution (100 mL) was added to the residue, and the mixture was extracted with ethyl acetate. The extract was washed with saturated brine, dried over sodium sulfate, and concentrated under reduced pressure. The obtained solid was washed with hexane to give the title compound (15.9 g, yield 78%) as a pale-yellow solid.

¹H NMR (300 MHz, CDCl₃) δ 3.36-3.50 (4H, m), 3.71-3.87 (4H, m), 6.53 (1H, d, J=8.7 Hz), 7.56 (1H, dd, J=9.1, 2.3 Hz), 8.21 (1H, d, J=2.7 Hz).

MS m/z 243 (M+H)⁺.

### Reference Example 149

### (6-morpholin-4-ylpyridin-3-yl)boronic acid

In the same manner as in Reference Example 147, the title compound was obtained as a pale-yellow solid from 4-(5-bromopyridin-2-yl)morpholine. yield 59%.

¹H NMR (300 MHz, DMSO-d₆) δ 3.41-3.54 (4H, m), 3.61-3.76 (4H, m), 6.76 (1H, d, J=8.7 Hz), 7.78-7.93 (1H, m), 8.48 (1H, d, J=1.3 Hz).

### Reference Example 150

### 4-(5-bromo-3-fluoropyridin-2-yl)morpholine

In the same manner as in Reference Example 146, the title compound was obtained as a white solid from 5-bromo-2-chloro-3-fluoropyridine and morpholine. yield 80%.

¹H NMR (300 MHz, CDCl₃) δ 3.42-3.50 (4H, m), 3.78-3.86 (4H, m), 7.39 (1H, dd, J=12.1, 2.3 Hz), 8.03-8.09 (1H, m).

### Reference Example 151

### (5-fluoro-6-morpholin-4-ylpyridin-3-yl)boronic acid

In the same manner as in Reference Example 147, the title compound was obtained as a brown solid from 4-(5-bromo-3-fluoropyridin-2-yl)morpholine. yield 39%.

MS m/z 227 (M+H)⁺.

### Reference Example 152

### methyl [(3S)-6-{[3-(5-fluoro-6-morpholin-4-ylpyridin-3-yl)-2-methyl-4-(trifluoromethyl)benzyl](trifluoroacetyl)amino}-2,3-dihydro-1-benzofuran-3-yl]acetate

In the same manner as in Example 184, the title compound was obtained as a pale-yellow oil from methyl [(3S)-6-{[2-methyl-4-(trifluoromethyl)-3-{[(trifluoromethyl)sulfonyl]oxy}benzyl](trifluoroacetyl)amino}-2,3-dihydro-1-benzofuran-3-yl]acetate and (5-fluoro-6-morpholin-4-ylpyridin-3-yl)boronic acid. yield 72%.

¹H NMR (300 MHz, CDCl₃) δ 2.04 (3H, s), 2.54-2.66 (1H, m), 2.71-2.83 (1H, m), 3.51-3.61 (4H, m), 3.72 (3H, s), 3.81-3.92 (5H, m), 4.27-4.35 (1H, m), 4.81 (1H, t, J=9.2 Hz), 5.00 (2H, s), 6.50 (1H, s), 6.58 (1H, d, J=7.9 Hz), 7.00-7.13 (2H, m), 7.30 (1H, d, J=7.9 Hz), 7.56 (1H, d, J=8.3 Hz), 7.74 (1H, s).

### Reference Example 153

### ethyl 3-hydroxypyridine-2-carboxylate

A mixed solution of 3-hydroxypyridine-2-carboxylic acid (8.00 g, 57.5 mmol) and concentrated sulfuric acid (4 mL) in ethanol (160 mL) and toluene (40 mL) was heated under reflux for 16 hr using a Dean-Stark apparatus. The reaction mixture was concentrated under reduced pressure to about ¼, an aqueous sodium bicarbonate solution was slowly added to basify the reaction mixture, and the mixture was extracted with ethyl acetate. The extract was dried over sodium sulfate, and concentrated under reduced pressure. The residue was purified by silica gel column chromatography (hexane:ethyl acetate=95:5-75:25) to give the title compound (5.12 g, yield 53%) as a pale-yellow oil.

¹H NMR (300 MHz, CDCl₃) δ 1.49 (3H, t, J=7.2 Hz), 4.54 (2H, q, J=6.9 Hz), 7.34-7.46 (2H, m), 8.30 (1H, dd, J=4.2, 1.9 Hz), 10.77 (1H, s).

MS m/z 168 (M+H)⁺.

### Reference Example 154

### ethyl 3-(2-ethoxy-1-methyl-2-oxoethoxy)pyridine-2-carboxylate

A mixed solution of ethyl 3-hydroxypyridine-2-carboxylate (5.12 g, 30.6 mmol), ethyl 2-bromopropionate (4.77 mL, 36.8 mmol) and potassium carbonate (8.47 g, 61.3 mmol) in N,N-dimethylformamide (80 mL) was stirred at 90° C. for 16 hr. The reaction mixture was concentrated under reduced pressure, water was added to the residue, and the mixture was extracted with ethyl acetate. The extract was dried over sodium sulfate, and concentrated under reduced pressure. The residue was purified by silica gel column chromatography (hexane:ethyl acetate=90:10-50:50) to give the title compound (6.01 g, yield 73%) as a yellow oil.

¹H NMR (300 MHz, CDCl₃) δ 1.24 (3H, t, J=7.2 Hz), 1.43 (3H, t, J=7.2 Hz), 1.68 (3H, d, J=6.8 Hz), 4.17-4.26 (2H, m), 4.46 (2H, q, J=7.2 Hz), 4.77 (1H, q, J=6.8 Hz), 7.22-7.29 (1H, m), 7.31-7.39 (1H, m), 8.31-8.35 (1H, m).

MS m/z 268 (M+H)⁺.

### Reference Example 155

### 2-methylfuro[3,2-b]pyridin-3-ol

To a solution of ethyl 3-(2-ethoxy-1-methyl-2-oxoethoxy)pyridine-2-carboxylate (6.01 g, 22.5 mmol) in toluene (45 mL) was added sodium ethoxide (1.91 g, 28.1 mmol), and the mixture was heated under reflux for 16 hr. The reaction mixture was cooled to room temperature, diluted with water (160 mL), and acidified with acetic acid. Then, the mixture was neutralized with saturated aqueous sodium hydrogen carbonate and 1 M aqueous sodium hydroxide solution, and extracted with ethyl acetate. The extract was dried over sodium sulfate, and concentrated under reduced pressure. The obtained solid was washed with diisopropyl ether-ethyl acetate to give the title compound (1.45 g, yield 43%) as a brown solid.

¹H NMR (300 MHz, CDCl₃) δ 2.54 (3H, s), 7.14 (1H, dd, J=8.3, 4.9 Hz), 7.62 (1H, dd, J=8.1, 1.3 Hz), 8.38 (1H, dd, J=4.9, 1.1 Hz).

### Reference Example 156

### 2-methylfuro[3,2-b]pyridin-3-yl trifluoromethanesulfonate

To a solution of 2-methylfuro[3,2-b]pyridin-3-ol (0.483 g, 3.24 mmol) in pyridine (8 mL) was added dropwise trifluoromethanesulfonic anhydride (0.820 mL, 4.86 mmol) at 0° C. The reaction mixture was warmed to room temperature, stirred for 16 hr, and concentrated under reduced pressure. Water was added to the residue, and the mixture was extracted with ethyl acetate. The extract was washed with water, dried over sodium sulfate, and concentrated under reduced pressure. The residue was purified by silica gel column chromatography (hexane:ethyl acetate=100:0-65:35) to give the title compound (0.466 g, yield 51%) as a pale-yellow oil.

¹H NMR (300 MHz, CDCl₃) δ 2.58 (3H, s), 7.23-7.30 (1H, m), 7.70 (1H, dd, J=8.7, 1.5 Hz), 8.60 (1H, dd, J=4.7, 1.3 Hz).

MS m/z 282 (M+H)⁺.

### Reference Example 157

### 2-methylfuro[2,3-b]pyridin-3(2H)-one

To a suspension of sodium hydride (60% in oil, 6.68 g, 0.167 mol) in 1,2-dimethoxyethane (100 mL) was added dropwise ethyl lactate (16.0 mL, 0.140 mol) at 00° C. over 20 min. The reaction mixture was stirred at room temperature for 30 min, and ethyl 2-chloronicotinate (10.0 g, 53.9 mmol) was added at room temperature over 10 min. The reaction mixture was heated to 80° C., stirred for 16 hr, and concentrated under reduced pressure. The residue was dissolved in water, washed with toluene, acetic acid was added and, under acidic conditions, the mixture was extracted with ethyl acetate. The extract was washed with water, dried over sodium sulfate, and concentrated under reduced pressure to give the title compound (4.50 g, yield 56%) as a red oil.

¹H NMR (300 MHz, CDCl₃) δ 1.60 (3H, d, J=7.2 Hz), 4.80 (1H, d, J=7.2 Hz), 7.13 (1H, dd), 8.04 (1H, dd, J=7.4, 2.1 Hz), 8.59 (1H, dd, J=4.9, 2.3 Hz).

MS m/z 150 (M+H)⁺.

### Reference Example 158

### 2-methyl-2,3-dihydrofuro[2,3-b]pyridin-3-yl acetate

To a mixed solution of 2-methylfuro[2,3-b]pyridin-3(2H)-one (4.50 g, 30.2 mmol) in tetrahydrofuran (40 mL) and methanol (20 mL) was added at 00° C. sodium borohydride (1.14 g, 30.2 mmol), and the mixture was stirred at the same temperature for 45 min. The reaction mixture was concentrated under reduced pressure, diluted with brine, and the mixture was extracted with ethyl acetate. The extract was dried over sodium sulfate, and concentrated under reduced pressure to give crude 2-methyl-2,3-dihydrofuro[2,3-b]pyridin-3-ol (3.30 g) as a yellow oil. The obtained crude 2-methyl-2,3-dihydrofuro[2,3-b]pyridin-3-ol (3.30 g, 21.8 mmol) was dissolved in pyridine (30 mL), and acetic anhydride (30 mL, 318 mmol) was added at room temperature. The reaction mixture was stirred for 16 hr, and concentrated under reduced pressure. The residue was dissolved in ethyl acetate, washed with water, dried over sodium sulfate, and concentrated under reduced pressure to give the title compound (3.20 g, yield 55% (2 steps)) as a yellow oil.

¹H NMR (300 MHz, CDCl₃) δ 1.44-1.56 (3H, m), 2.09 (3H, s), 4.76-4.90 (1H, m), 5.82-6.17 (1H, m), 6.84-6.93 (1H, m), 7.71-7.79 (1H, m), 8.16-8.23 (1H, m).

MS m/z 194 (M+H)⁺.

### Reference Example 159

### 2-methylfuro[2,3-b]pyridine

To 2-methyl-2,3-dihydrofuro[2,3-b]pyridin-3-yl acetate (3.13 g, 16.2 mmol) was added polyphosphoric acid (30 g), and the mixture was stirred at 100° C. for 16 hr. The reaction mixture was cooled to room temperature, ice was slowly added, and the mixture was alkalified with 28% aqueous ammonia solution and extracted with ethyl acetate. The extract was dried over sodium sulfate, and concentrated under reduced pressure to give the title compound (2.15 g, yield 100%) as a brown oil.

¹H NMR (300 MHz, CDCl₃) δ 2.49 (3H, d, J=1.1 Hz), 6.38 (1H, q, J=1.1 Hz), 7.15 (1H, dd, J=7.6, 4.9 Hz), 7.78 (1H, dd, J=7.6, 1.9 Hz), 8.21 (1H, dd, J=4.9, 1.9 Hz).

### Reference Example 160

### 3-bromo-2-methylfuro[2,3-b]pyridine

A solution of 2-methylfuro[2,3-b]pyridine (0.504 g, 3.79 mmol) in dichloromethane (4 mL) was cooled to −15° C., a solution of bromine (0.638 mL, 13.3 mmol) in dichloromethane (3 mL) was slowly added dropwise. The reaction mixture was stirred at room temperature for 6 hr, and concentrated under reduced pressure. The residue was diluted with 1 M sodium hydroxide, and the mixture was extracted with ethyl acetate. The extract was dried over sodium sulfate, and concentrated under reduced pressure. The residue was purified by silica gel column chromatography (hexane:ethyl acetate=100:0-75:25) to give the title compound (0.432 g, yield 54%) as a pale-yellow oil.

¹H NMR (300 MHz, CDCl₃) δ 2.52 (3H, s), 7.22-7.29 (1H, m), 7.76 (1H, dd, J=7.9, 1.9 Hz), 8.28 (1H, dd, J=4.9, 1.9 Hz).

### Reference Example 161

### methyl {(3S)-6-[(4-bromo-2,3-dihydro-1H-inden-1-yl)amino]-2,3-dihydro-1-benzofuran-3-yl}acetate

To a mixed solution of 4-bromoindan-1-one (1.00 g, 4.74 mmol) and methyl [(3S)-6-amino-2,3-dihydro-1-benzofuran-3-yl]acetate (1.03 g, 4.97 mmol) in methanol (10 mL), acetic acid (1 mL) and tetrahydrofuran (2 mL) was added at 0° C. borane-2-picoline complex (0.640 g, 5.69 mmol), and the mixture was stirred at room temperature for 24 hr. The reaction mixture was concentrated under reduced pressure, 1 M aqueous hydrochloric acid solution and ethyl acetate were added, and the mixture was stirred for 30 min. Then, sodium carbonate was slowly added to neutralize the reaction mixture, and the mixture was extracted with ethyl acetate. The extract was dried over sodium sulfate, and concentrated under reduced pressure. The residue was purified by silica gel column chromatography (hexane:ethyl acetate=100:0-75:25) to give the title compound (1.75 g, yield 92%) as a pale-yellow oil.

¹H NMR (300 MHz, CDCl₃) δ 1.81-1.99 (1H, m), 2.48-2.66 (2H, m), 2.75 (1H, dd, J=16.7, 5.7 Hz), 2.80-2.96 (1H, m), 2.96-3.13 (1H, m), 3.67-3.85 (4H, m), 3.94 (1H, br s), 4.24 (1H, dd, J=9-0.3, 5.9 Hz), 4.73 (1H, t, J=8.9 Hz), 5.03 (1H, t, J=6.8 Hz), 6.15-6.23 (2H, m), 6.95 (1H, d, J=8.7 Hz), 7.07 (1H, t, J=7.8 Hz), 7.29 (1H, d, J=6.8 Hz), 7.41 (1H, d, J=8.0 Hz).

MS m/z 402 (M+H)⁺.

### Reference Example 162

### methyl {(3S)-6-[(4-bromo-2,3-dihydro-1H-inden-1-yl)(trifluoroacetyl)amino]-2,3-dihydro-1-benzofuran-3-yl}acetate

A mixed solution of methyl {(3S)-6-[(4-bromo-2,3-dihydro-1H-inden-1-yl)amino]-2,3-dihydro-1-benzofuran-3-yl}acetate (1.75 g, 4.35 mmol) and triethylamine (0.910 mL, 6.53 mmol) was cooled to 0° C., and trifluoroacetic anhydride (0.738 mL, 5.23 mmol) was slowly added dropwise. The reaction mixture was warmed to room temperature, and stirred for 1.5 hr. The reaction mixture was poured into saturated aqueous sodium hydrogen carbonate, and the mixture was extracted with ethyl acetate. The extract was dried over sodium sulfate, and concentrated under reduced pressure. The residue was purified by silica gel column chromatography (hexane:ethyl acetate=100:0-70:30) to give the title compound (1.51 g, yield 70%) as a yellow oil.

¹H NMR (300 MHz, CDCl₃) δ 1.91-2.08 (1H, m), 2.55-2.83 (3H, m), 2.84-3.01 (1H, m), 3.02-3.18 (1H, m), 3.69-3.91 (4H, m), 4.31-4.43 (1H, m), 4.80-4.91 (1H, m), 5.13 (1H, q, J=7.0 Hz), 6.33 (1H, s), 7.11 (1H, t, J=7.7 Hz), 7.23-7.33 (1H, m), 7.44 (1H, d, J=7.9 Hz), 7.54-7.60 (1H, m), 9.50 (1H, d, J=7.2 Hz).

### Reference Example 163

### optically active form (A) of methyl {(3S)-6-[(4-bromo-2,3-dihydro-1H-inden-1-yl)(trifluoroacetyl)amino]-2,3-dihydro-1-benzofuran-3-yl}acetate

Methyl {(3S)-6-[(4-bromo-2,3-dihydro-1H-inden-1-yl)(trifluoroacetyl)amino]-2,3-dihydro-1-benzofuran-3-yl}acetate (1.33 g) was resolved by preparative HPLC to give the title compound (0.633 g, recovery rate 96%, enantiometric excess 99.9% d.e.) as a pale-yellow oil.

(high performance liquid chromatography conditions)
column: CHIRALCEL OJ (manufactured by Daicel Chemical Industries, Ltd.)
mobile phase: hexane/isopropyl alcohol (volume ratio: 90/10)
flow rate: 80 mL/min
detection: UV (254 nm)
temperature: 30° C.
retention time: 7.6 min (area ratio: 99.90%)

### Reference Example 164

### optically active form (B) of methyl {(3S)-6-[(4-bromo-2,3-dihydro-1H-inden-1-yl)(trifluoroacetyl)amino]-2,3-dihydro-1-benzofuran-3-yl}acetate

Methyl {(3S)-6-[(4-bromo-2,3-dihydro-1H-inden-1-yl)(trifluoroacetyl)amino]-2,3-dihydro-1-benzofuran-3-yl}acetate (1.33 g) was resolved by preparative HPLC to give the title compound (0.609 g, recovery rate 92%, enantiometric excess 99.9% d.e.) as a pale-yellow oil.

(high performance liquid chromatography conditions)
column: CHIRALCEL OJ (manufactured by Daicel Chemical Industries, Ltd.)
mobile phase: hexane/isopropyl alcohol (volume ratio: 90/10)
flow rate: 80 mL/min
detection: UV (254 nm)
temperature: 30° C.
retention time: 9.9 min (area ratio: 99.90%)

### Reference Example 165

### methyl {(3S)-6-[{4-[(5-fluoro-2-nitrophenyl)amino]-2,3-dihydro-1H-inden-1-yl}(trifluoroacetyl)amino]-2,3-dihydro-1-benzofuran-3-yl}acetate

Methyl {(3S)-6-[(4-bromo-2,3-dihydro-1H-inden-1-yl)(trifluoroacetyl)amino]-2,3-dihydro-1-benzofuran-3-yl}acetate (1.51 g, 3.04 mmol), 5-fluoro-2-nitroaniline (0.522 g, 3.34 mmol) and tripotassium phosphate (1.29 g, 6.08 mmol) were suspended in toluene (20 mL) and, after argon substitution, tris(dibenzylideneacetone)dipalladium (0) (0.139 g, 0.152 mmol) and 2-dicyclohexylphosphino-2′,4′,6′-triisopropylbiphenyl (0.145 g, 0.304 mmol) were added. The reaction mixture was stirred under an argon atmosphere at 105° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=100:0-70:30) to give the title compound (1.64 g, yield 94%) as a brown non-crystalline powder.

MS m/z 572 (M−H)⁻.

### Reference Example 166

### methyl {(3S)-6-[{4-[(2-amino-5-fluorophenyl)amino]-2,3-dihydro-1H-inden-1-yl}(trifluoroacetyl)amino]-2,3-dihydro-1-benzofuran-3-yl}acetate

Methyl {(3S)-6-[{4-[(5-fluoro-2-nitrophenyl)amino]-2,3-dihydro-1H-inden-1-yl}(trifluoroacetyl)amino]-2,3-dihydro-1-benzofuran-3-yl}acetate (1.64 g, 2.86 mmol) was dissolved in methanol (15 mL) and tetrahydrofuran (5 mL), 10% palladium-carbon (50% water-containing product, 0.4 g) was added, and the mixture was stirred under a hydrogen atmosphere (balloon pressure) at room temperature for 4 hr. The catalyst was filtered off, and the filtrate was concentrated under reduced pressure to give the title compound (1.47 g, yield 95%) as a purple non-crystalline powder.

MS m/z 544 (M+H)⁺.

### Reference Example 167

### methyl [(3S)-6-{[4-(6-fluoro-2-methyl-1H-benzimidazol-1-yl)-2,3-dihydro-1H-inden-1-yl](trifluoroacetyl)amino}-2,3-dihydro-1-benzofuran-3-yl]acetate

A mixed solution of methyl {(3S)-6-[{4-[(2-amino-5-fluorophenyl)amino]-2,3-dihydro-1H-inden-1-yl}(trifluoroacetyl)amino]-2,3-dihydro-1-benzofuran-3-yl}acetate (0.300 g, 0.552 mmol) in acetic acid (4 mL) and acetic anhydride (2 mL) was heated under reflux for 16 hr. The reaction mixture was concentrated under reduced pressure, diluted with saturated aqueous sodium hydrogen carbonate, and extracted with ethyl acetate. The extract was dried over sodium sulfate, and concentrated under reduced pressure. The residue was purified by silica gel column chromatography (hexane:ethyl acetate=90:10-0:100) to give the title compound (0.315 g, yield 100%) as a pale-yellow oil.

MS m/z 568 (M+H)⁺.

### Reference Example 168

### methyl [(3S)-6-{[4-(2-ethyl-6-fluoro-1H-benzimidazol-1-yl)-2,3-dihydro-1H-inden-1-yl](trifluoroacetyl)amino}-2,3-dihydro-1-benzofuran-3-yl]acetate

A solution of methyl {(3S)-6-[{4-[(2-amino-5-fluorophenyl)amino]-2,3-dihydro-1H-inden-1-yl}(trifluoroacetyl)amino]-2,3-dihydro-1-benzofuran-3-yl}acetate (0.590 g, 1.30 mmol) in N,N-dimethylacetamide (5 mL) was cooled to 0° C., and propionyl chloride (0.114 mL, 1.30 mmol) was slowly added. The reaction mixture was stirred at room temperature for 5 hr, poured into saturated aqueous sodium hydrogen carbonate, and the mixture was extracted with ethyl acetate. The extract was dried over sodium sulfate, and concentrated under reduced pressure. Then, the residue was dissolved in acetic acid (8 mL) and the mixture was stirred for 16 hr with heating under reflux, and concentrated under reduced pressure. To the residue was added saturated aqueous sodium hydrogen carbonate, and the mixture was extracted with ethyl acetate. The extract was dried over sodium sulfate, and concentrated under reduced pressure. The residue was purified by silica gel column chromatography (hexane:ethyl acetate=90:10-20:80) to give the title compound (0.617 mg, yield 98%) as a pale-red non-crystalline powder.

MS m/z 582 (M+H)⁺.

### Reference Example 169

### methyl [(3S)-6-{[4-(2-ethoxy-6-fluoro-1H-benzimidazol-1-yl)-2,3-dihydro-1H-inden-1-yl](trifluoroacetyl)amino}-2,3-dihydro-1-benzofuran-3-yl]acetate

Methyl {(3S)-6-[{4-[(2-amino-5-fluorophenyl)amino]-2,3-dihydro-1H-inden-1-yl}(trifluoroacetyl)amino]-2,3-dihydro-1-benzofuran-3-yl}acetate (0.567 g, 1.04 mmol) and tetraethoxymethane (0.654 mL, 3.13 mmol) were dissolved in acetic acid (6 mL), and the mixture was stirred at 60° C. for 1 hr. The reaction mixture was concentrated under reduced pressure, saturated aqueous sodium hydrogen carbonate was added to the obtained residue, and the mixture was extracted with ethyl acetate. The extract was dried over sodium sulfate, and concentrated under reduced pressure. The residue was purified by silica gel column chromatography (hexane:ethyl acetate=90:10-50:50) to give the title compound (0.553 g, yield 89%) as a pale-yellow non-crystalline powder.

MS m/z 598 (M+H)⁺.

### Reference Example 170

### methyl 3-(2-ethyl-7-methyl-1H-benzimidazol-1-yl)-2-methylbenzoate

In the same manner as in Reference Example 168, the title compound was obtained as a purple oil from methyl 3-[(2-amino-6-methylphenyl)amino]-2-methylbenzoate. yield 82%.

¹H NMR (300 MHz, CDCl₃) δ 1.33 (3H, t, J=7.5 Hz), 1.82 (3H, s), 2.15 (3H, s), 2.54 (2H, q, J=7.5 Hz), 3.95 (3H, s), 6.88-6.95 (1H, m), 7.16 (1H, t, J=7.7 Hz), 7.37-7.50 (2H, m), 7.66 (1H, d, J=7.9 Hz), 8.07 (1H, dd, J=7.5, 1.9 Hz).

MS m/z 309 (M+H)⁺.

### Reference Example 171

### methyl {(3S)-6-[{3-[(5-fluoropyridin-2-yl)amino]-2-methylbenzyl}(trifluoroacetyl)amino]-2,3-dihydro-1-benzofuran-3-yl}acetate

A solution of methyl {(3S)-6-[(3-bromo-2-methylbenzyl)(trifluoroacetyl)amino]-2,3-dihydro-1-benzofuran-3-yl}acetate (0.380 g, 0.781 mmol), 5-fluoropyridin-2-amine (0.105 g, 0.938 mmol) and cesium carbonate (0.509 g, 1.56 mmol) in toluene (5 mL) was substituted with argon, and tris(dibenzylideneacetone)dipalladium (0) (28.6 mg, 0.0310 mmol) and 4,5-bis(diphenylphosphino)-9,9-dimethylxanthene (36.2 mg, 0.0630 mmol) were added. The reaction mixture was stirred under an argon atmosphere at 105° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-65:35), and then preparative HPLC (water:acetonitrile=60:40-0:100) to give the title compound (0.243 g, yield 60%) as a pale-yellow oil.

¹H NMR (300 MHz, CDCl₃) δ 2.00 (3H, s), 2.57 (1H, dd, J=16.6, 8.7 Hz), 2.75 (1H, dd, J=16.6, 5.7 Hz), 3.71 (3H, s), 3.77-3.94 (1H, m), 4.28 (1H, dd, J=9.4, 6.4 Hz), 4.78 (1H, t, J=9.2 Hz), 4.99 (2H, s), 6.14 (1H, s), 6.38-6.57 (3H, m), 6.95 (1H, d, J=7.5 Hz), 7.01-7.16 (2H, m), 7.17-7.33 (2H, m), 8.03 (1H, d, J=3.0 Hz).

MS m/z 518 (M+H)⁺.

### Reference Example 172

### 3-bromo-5-fluoro-2-methyl-1H-indole

To a solution of 5-fluoro-2-methyl-1H-indole (1.50 g, 10.1 mmol) in dichloromethane (30 mL) was slowly added dropwise a solution of bromine (1.69 g, 10.6 mmol) in dichloromethane (3 mL). The reaction mixture was stirred at room temperature for 6 hr, and concentrated under reduced pressure. To the residue was added 1 M aqueous sodium hydroxide solution, and the mixture was extracted with ethyl acetate. The extract was dried over sodium sulfate, and concentrated under reduced pressure. The residue was purified by silica gel column chromatography (hexane:ethyl acetate=100:0-75:25) to give the title compound (1.95 g, yield 85%) as a pale-yellow oil.

¹H NMR (300 MHz, CDCl₃) δ 2.41 (3H, s), 6.82-6.95 (1H, m), 7.07-7.20 (2H, m), 8.06 (1H, br s).

### Reference Example 173

### 3-bromo-5-fluoro-2-methyl-1-[3-(methylsulfonyl)propyl]-1H-indole

To a solution of 3-bromo-5-fluoro-2-methyl-1H-indole (1.05 g, 4.59 mmol) in N,N-dimethylformamide (14 mL) was slowly added at 0° C. sodium hydride (60% in oil, 0.220 g, 5.51 mmol), and the mixture was stirred at the same temperature for min. Further, 3-(methylsulfonyl)propyl 4-methylbenzenesulfonate (1.48 g, 5.05 mmol) obtained according to WO2007/018314 was slowly added at 0° C. The reaction mixture was stirred at room temperature for 16 hr, water was added, and the mixture was extracted with ethyl acetate. The extract was washed with water, dried over anhydrous sodium sulfate, and concentrated under reduced pressure. The residue was purified by silica gel column chromatography (hexane:ethyl acetate=90:10-20:80) to give the title compound (0.837 g, yield 55%) as a red oil.

¹H NMR (300 MHz, CDCl₃) δ 2.24-2.38 (2H, m), 2.45 (3H, s), 2.90 (3H, s), 2.97 (2H, t, J=6.8 Hz), 4.32 (2H, t, J=7.0 Hz), 6.90-7.00 (1H, m), 7.12-7.27 (2H, m).

MS m/z 348 (M+H)⁺.

### Reference Example 174

### methyl [(3S)-6-{[3-(4,6-dimethyl-2-morpholin-4-ylpyrimidin-5-yl)-2-methylbenzyl](trifluoroacetyl)amino}-2,3-dihydro-1-benzofuran-3-yl]acetate

Methyl {(3S)-6-[(3-bromo-2-methylbenzyl)(trifluoroacetyl)amino]-2,3-dihydro-1-benzofuran-3-yl}acetate (4.06 g, 8.35 mmol), and (4,6-dimethyl-2-morpholin-4-ylpyrimidin-5-yl)boronic acid (2.38 g, 10.0 mmol) were suspended in a mixed solution of 2 M aqueous sodium carbonate solution (10.0 mL) and toluene (50 mL) and, after argon substitution, tris(dibenzylideneacetone)dipalladium (0) (0.306 g, 0.334 mmol) and 2-dicyclohexylphosphino-2′,6′-dimethoxybiphenyl (0.565 g, 1.34 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-60:40) to give the title compound (3.63 g, yield 73%) as a white non-crystalline powder.

¹H NMR (300 MHz, CDCl₃) δ 1.70 (3H, s), 1.93 (6H, s), 2.56 (1H, dd, J=16.7, 9.1 Hz), 2.74 (1H, dd, J=15.9, 4.9 Hz), 3.71 (3H, s), 3.73-3.91 (9H, m), 4.25 (1H, dd, J=9.1, 6.8 Hz), 4.76 (1H, t, J=9.1 Hz), 5.02 (2H, s), 6.37 (1H, s), 6.48 (1H, d, J=8.0 Hz), 6.94 (1H, d, J=6.8 Hz), 7.02 (1H, d, J=7.6 Hz), 7.11-7.23 (2H, m).

MS m/z 599 (M+H)⁺.

### Reference Example 175

### methyl 3-(2-ethoxy-6-fluoro-1H-benzimidazol-1-yl)-2-methylbenzoate

A solution of methyl 3-[(2-amino-5-fluorophenyl)amino]-2-methylbenzoate (2.0 g, 7.29 mmol) and tetraethyl orthocarbonate (4.23 g, 22 mmol) in acetic acid (20 mL) was stirred at 60° C. for 3 hr. The solvent was concentrated, the residue was poured into an aqueous sodium hydrogen carbonate solution, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=90:10-75:25) to give the title compound (1.92 g, yield 80%) as an oil.

¹H NMR (300 MHz, CDCl₃) δ 1.39 (3H, t, J=7.2 Hz), 2.25 (3H, s), 3.94 (3H, s), 4.52-4.64 (2H, m), 6.51-6.54 (1H, m), 6.89-6.96 (1H, m), 7.40-7.42 (2H, m), 7.47-7.52 (1H, m), 8.00-8.03 (1H, m).

MS m/z 329 (M+H)⁺.

### Reference Example 176

### [3-(2-ethoxy-6-fluoro-1H-benzimidazol-1-yl)-2-methylphenyl]methanol

Lithium aluminum hydride (2.0 M tetrahydrofuran solution, 4.5 mL, 9.0 mmol) was added dropwise at 0° C. to a solution of methyl 3-(2-ethoxy-6-fluoro-1H-benzimidazol-1-yl)-2-methylbenzoate (1.92 g, 5.83 mmol) in tetrahydrofuran (200 mL), and the mixture was stirred at 0° C. for 1 hr. To the reaction mixture was added sodium sulfate 10 hydrate, and the mixture was stirred at room temperature for 1 hr. The reaction mixture was filtered through celite, and concentrated under reduced pressure to give the title compound (1.80 g, yield quant.) as an oil.

¹H NMR (300 MHz, CDCl₃) δ 1.39 (3H, t, J=7.2 Hz), 2.01 (3H, s), 3.00 (1H, br), 4.50-4.61 (2H, m), 4.80 (2H, s), 6.51-6.55 (1H, m), 6.86-6.94 (1H, m), 7.18-7.21 (1H, m), 7.32-7.37 (1H, m), 7.43-7.48 (1H, m), 7.56-7.58 (1H, m).

### Reference Example 177

### methyl 2-methyl-3-{[2-nitro-4-(trifluoromethyl)phenyl]amino}benzoate

A suspension of 1-bromo-2-nitro-4-(trifluoromethyl)benzene (8.1 g, 30 mmol), methyl 3-amino-2-methylbenzoate (4.13 g, 25 mmol), tris(dibenzylideneacetone)dipalladium (0) (800 mg, 0.87 mmol), bis(2-diphenylphosphinophenyl)ether (942 mg, 1.75 mmol) and tripotassium phosphate (15.9 g, 75 mmol) in toluene (100 mL) was stirred overnight under an argon atmosphere at 105° C. Water was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=90:10-30:70) to give the title compound (8.0 g, yield 90%) as a solid.

¹H NMR (300 MHz, CDCl₃) δ 2.47 (3H, s), 3.94 (3H, s), 6.74 (1H, d, J=9.0 Hz), 7.34-7.44 (2H, m), 7.49-7.52 (1H, m), 7.85-7.88 (1H, m), 8.52 (1H, d, J=1.2 Hz), 9.55 (1H, s).

### Reference Example 178

### methyl 3-{[2-amino-4-(trifluoromethyl)phenyl]amino}-2-methylbenzoate

A suspension of methyl 2-methyl-3-{[2-nitro-4-(trifluoromethyl)phenyl]amino}benzoate (8.0 g, 22.5 mmol) and palladium-carbon (1.0 g) in methanol (300 mL) was stirred under a hydrogen atmosphere overnight at room temperature. The reaction mixture was filtered and concentrated under reduced pressure to give the title compound (7.3 g, yield quant.) as a solid.

¹H NMR (300 MHz, CDCl₃) δ2.45 (3H, s), 3.78 (2H, s), 3.91 (3H, s), 5.28 (1H, s), 6.86-6.93 (2H, m), 6.97-7.02 (2H, m), 7.15 (1H, t, J=8.1 Hz), 7.43-7.46 (1H, m).

### Reference Example 179

### methyl 2-methyl-3-{[2-{[(2R)-tetrahydrofuran-2-ylcarbonyl]amino}-4-(trifluoromethyl)phenyl]amino}benzoate

A solution of methyl 3-{[2-amino-4-(trifluoromethyl)phenyl]amino}-2-methylbenzoate (7.3 g, 22.5 mmol), (2R)-tetrahydrofuran-2-ylcarboxylic acid (2.79 g, 24 mmol), 1-ethyl-3-(3-dimethylaminopropyl)carbodiimide hydrochloride (5.18 g, 27 mmol) and 1-hydroxy-1H-benzotriazole monohydrate (4.13 g, 27 mmol) in acetonitrile (200 mL) was stirred overnight at room temperature. Water was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=80:20-50:50) to give the title compound (9.22 g, yield 97%) as an oil.

¹H NMR (300 MHz, CDCl₃) δ1.75-1.84 (1H, m), 1.87-1.96 (1H, m), 2.06-2.13 (1H, m), 2.29-2.38 (1H, m), 2.46 (3H, s), 3.73-3.89 (2H, m), 3.91 (3H, s), 4.45-4.49 (1H, m), 6.29 (1H, s), 6.99-7.03 (2H, m), 7.14 (1H, t, J=8.1 Hz), 7.33 (1H, d, J=8.7 Hz), 7.48 (1H, d, J=7.8 Hz), 7.85 (1H, s), 8.67 (1H, s).

### Reference Example 180

### methyl 2-methyl-3-{2-[(2R)-tetrahydrofuran-2-yl]-5-(trifluoromethyl)-1H-benzimidazol-1-yl}benzoate

Methyl 2-methyl-3-{[2-{[(2R)-tetrahydrofuran-2-ylcarbonyl]amino}-4-(trifluoromethyl)phenyl]amino}benzoate (9.22 g, 21.8 mmol) and polyphosphoric acid (18 g) were stirred at 130° C. for 17 min, and the reaction container was cooled to room temperature. Water was added to the reaction mixture, and the mixture was extracted with ethyl acetate. The extract was washed with saturated brine, dried over anhydrous magnesium sulfate, and concentrated under reduced pressure. The residue was purified by basic silica gel column chromatography (hexane:ethyl acetate=90:10-75:25) to give the title compound (5.76 g, yield 65%) as an oil.

¹H NMR (300 MHz, CDCl₃) δ1.89-2.00 (1H, m), 2.12-2.17 (3H, m), 2.18-2.27 (2H, m), 2.51-2.62 (1H, m), 3.78-3.90 (2H, m), 3.95 (3H, s), 4.79-4.91 (1H, m), 6.96-6.99 (1H, m), 7.38-7.56 (3H, m), 8.07-8.10 (1H, m), 8.13 (1H, s).

### Reference Example 181

### (2-methyl-3-{2-[(2R)-tetrahydrofuran-2-yl]-5-(trifluoromethyl)-1H-benzimidazol-1-yl}phenyl)methanol

Lithium aluminum hydride (2.0 M tetrahydrofuran solution, 8.5 mL, 17 mmol) was added dropwise to a solution of methyl 2-methyl-3-{2-[(2R)-tetrahydrofuran-2-yl]-5-(trifluoromethyl)-1H-benzimidazol-1-yl}benzoate (5.76 g, 14.2 mmol) in tetrahydrofuran (250 mL) at 0° C., and the mixture was stirred at 0° C. for 1 hr. Sodium sulfate 10 hydrate was added to the reaction mixture at 0° C., and the mixture was stirred at room temperature for 1 hr. The reaction mixture was filtered through celite, and concentrated under reduced pressure to give the title compound (5.39 g, yield quant.) as an oil.

¹H NMR (300 MHz, CDCl₃) δ1.83-2.02 (4H, m), 2.13-2.27 (2H, m), 2.30-2.56 (1H, m), 3.69-3.87 (3H, m), 4.76-4.88 (3H, m), 6.96-6.99 (1H, m), 7.16-7.46 (3H, m), 7.64 (1H, t, J=7.2 Hz), 8.11 (1H, s).

### Reference Example 182

### 2-methyl-3-{2-[(2R)-tetrahydrofuran-2-yl]-5-(trifluoromethyl)-1H-benzimidazol-1-yl}benzaldehyde

To a solution of (2-methyl-3-{2-[(2R)-tetrahydrofuran-2-yl]-5-(trifluoromethyl)-1H-benzimidazol-1-yl}phenyl)methanol (1.58 g, 4.2 mmol) in acetonitrile (25 mL) was added Dess-Martin periodonane (2.0 g, 4.7 mmol) at 0° C., and the mixture was stirred at 0° C. for 0.5 hr. An aqueous sodium thiosulfate solution and an aqueous sodium hydrogen carbonate solution were added to the reaction mixture, and the mixture was stirred at room temperature for 30 min. The reaction mixture was extracted with ethyl acetate, and the extract was washed with saturated brine, dried over anhydrous magnesium sulfate, and concentrated under reduced pressure. The residue was purified by silica gel column chromatography (hexane:ethyl acetate=80:20-50:50) to give the title compound (0.866 g, yield 55%) as an oil.

¹H NMR (300 MHz, CDCl₃) δ1.91-2.00 (1H, m), 2.11-2.30 (5H, m), 2.58-2.65 (1H, m), 3.78-3.85 (2H, m), 4.82-4.94 (1H, m), 6.96-7.00 (1H, m), 7.46-7.67 (3H, m), 8.03-8.07 (1H, m), 8.13 (1H, s), 10.36-10.38 (1H, m).

### Reference Example 183

### methyl 2-methyl-3-{[2-nitro-4-(trifluoromethoxy)phenyl]amino}benzoate

A suspension of 2-nitro-4-(trifluoromethoxy)aniline (5.0 g, 22.5 mmol), methyl 3-bromo-2-methylbenzoate (4.81 g, 21 mmol), tris(dibenzylideneacetone)dipalladium (0) (800 mg, 0.87 mmol), 2-dicyclohexylphosphino-2′,4′,6′-triisopropylbiphenyl (834 mg, 1.75 mmol) and tripotassium phosphate (9.34 g, 44 mmol) in toluene (140 mL) was stirred at 105° C. overnight under an argon atmosphere. Water was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=98:2-90:10) to give the title compound (8.19 g, yield quant.) as a solid.

¹H NMR (300 MHz, CDCl₃) δ 2.47 (3H, s), 3.93 (3H, s), 6.71 (1H, d, J=9.3 Hz), 7.20-7.25 (1H, m), 7.31-7.43 (2H, m), 7.81-7.84 (1H, m), 8.12-8.13 (1H, m), 9.33 (1H, s).

### Reference Example 184

### methyl 3-{[2-amino-4-(trifluoromethoxy)phenyl]amino}-2-methylbenzoate

A suspension of methyl 2-methyl-3-{[2-nitro-4-(trifluoromethoxy)phenyl]amino}benzoate (8.19 g, 22.1 mmol) and palladium-carbon (1.0 g) in methanol (500 mL) was stirred at room temperature overnight under a hydrogen atmosphere. The reaction mixture was filtered and concentrated under reduced pressure to give the title compound (7.25 g, yield quant.) as a solid.

¹H NMR (300 MHz, CDCl₃) δ 2.46 (3H, s), 3.88 (2H, s), 3.90 (3H, s), 5.07 (1H, s), 6.57-6.61 (1H, m), 6.65-6.69 (1H, m), 6.91-6.94 (1H, m), 7.06-7.16 (1H, m), 7.20-7.26 (1H, m), 7.30-7.32 (1H, m).

### Reference Example 185

### methyl 2-methyl-3-{[2-{[(2R)-tetrahydrofuran-2-ylcarbonyl]amino}-4-(trifluoromethoxy)phenyl]amino}benzoate

A solution of methyl 3-{[2-amino-4-(trifluoromethoxy)phenyl]amino}-2-methylbenzoate (7.15 g, 21 mmol), (2R)-tetrahydrofuran-2-ylcarboxylic acid (2.44 g, 21 mmol), 1-ethyl-3-(3-dimethylaminopropyl)carbodiimide hydrochloride (4.79 g, 25 mmol) and 1-hydroxy-1H-benzotriazole monohydrate (3.83 g, 25 mmol) in acetonitrile (150 mL) was stirred overnight at room temperature. Water was added to the reaction mixture, and the mixture was extracted with ethyl acetate. The extract was washed with saturated brine, dried over anhydrous magnesium sulfate, and concentrated under reduced pressure. The residue was purified by basic silica gel column chromatography (hexane:ethyl acetate=80:20-50:50) to give the title compound (8.1 g, yield 88%) as a solid.

¹H NMR (300 MHz, CDCl₃) δ1.56-1.67 (1H, m), 1.76-1.85 (1H, m), 1.97-2.08 (1H, m), 2.20-2.32 (1H, m), 2.50 (3H, s), 3.40-3.47 (1H, m), 3.68-3.75 (1H, m), 3.91 (3H, s), 4.37-4.41 (1H, m), 5.60 (1H, s), 6.68 (1H, d, J=8.1 Hz), 6.96-7.00 (1H, m), 7.04-7.09 (2H, m), 7.35 (1H, d, J=7.8 Hz), 8.01 (1H, d, J=1.8 Hz), 8.83 (1H, s).

### Reference Example 186

### methyl 2-methyl-3-{2-[(2R)-tetrahydrofuran-2-yl]-5-(trifluoromethoxy)-1H-benzimidazol-1-yl}benzoate

Methyl 2-methyl-3-{[2-{[(2R)-tetrahydrofuran-2-ylcarbonyl]amino}-4-(trifluoromethoxy)phenyl]amino}benzoate (8.1 g, 18.48 mmol) and polyphosphoric acid (16 g) were stirred at 130° C. for 15 min. The reaction container was cooled to room temperature, water was added to the reaction mixture, and the mixture was extracted with ethyl acetate. The extract was washed with saturated brine, dried over anhydrous magnesium sulfate, and concentrated under reduced pressure. The residue was purified by basic silica gel column chromatography (hexane:ethyl acetate=90:10-80:20) to give the title compound (6.81 g, yield 88%) as an oil.

¹H NMR (300 MHz, CDCl₃) δ1.89-2.00 (1H, m), 2.09-2.25 (2H, m), 2.18 (3H, s), 2.49-2.61 (1H, m), 3.74-3.91 (2H, m), 3.95 (3H, s), 4.75-4.88 (1H, m), 6.85-6.88 (1H, m), 7.08-7.11 (1H, m), 7.36-7.55 (2H, m), 7.71 (1H, s), 8.05-8.08 (1H, m).

### Reference Example 187

### (2-methyl-3-{2-[(2R)-tetrahydrofuran-2-yl]-5-(trifluoromethoxy)-1H-benzimidazol-1-yl}phenyl)methanol

Lithium aluminum hydride (2.0 M tetrahydrofuran solution, mL, 20 mmol) was added dropwise to a solution of methyl 2-methyl-3-{2-[(2R)-tetrahydrofuran-2-yl]-5-(trifluoromethoxy)-1H-benzimidazol-1-yl}benzoate (6.83 g, 16.2 mmol) in tetrahydrofuran (200 mL) at 0° C., and the mixture was stirred at 0° C. for 1 hr. Sodium sulfate 10 hydrate was added to the reaction mixture at 0° C., and the mixture was stirred at room temperature for 1 hr. The reaction mixture was filtered through celite, and concentrated under reduced pressure to give the title compound (5.55 g, yield 87%) as an oil.

¹H NMR (300 MHz, CDCl₃) δ1.83-2.03 (4H, m), 2.14-2.27 (2H, m), 2.46-2.52 (1H, m), 3.72-3.88 (2H, m), 3.94-3.99 (1H, m), 4.82 (2H, s), 4.84-4.86 (1H, m), 6.89 (1H, d, J=8.7 Hz), 7.08-7.32 (2H, m), 7.37-7.43 (1H, m), 7.63-7.68 (1H, m), 7.73 (1H, s).

### Reference Example 188

### 2-methyl-3-{2-[(2R)-tetrahydrofuran-2-yl]-5-(trifluoromethoxy)-1H-benzimidazol-1-yl}benzaldehyde

To a solution of (2-methyl-3-{2-[(2R)-tetrahydrofuran-2-yl]-5-(trifluoromethoxy)-1H-benzimidazol-1-yl}phenyl)methanol (1.0 g, 2.55 mmol) in acetonitrile (10 mL) was added Dess-Martin periodonane (1.23 g, 2.9 mmol) at 0° C., and the mixture was stirred at 00° C. for 1 hr. An aqueous sodium thiosulfate solution and an aqueous sodium hydrogen carbonate solution were added to the reaction mixture, and the mixture was stirred at room temperature for 30 min. The reaction mixture was extracted with ethyl acetate, and the extract was washed with saturated brine, dried over anhydrous magnesium sulfate, and concentrated under reduced pressure. The residue was purified by silica gel column chromatography (hexane:ethyl acetate=95:5-60:40) to give the title compound (0.530 g, yield 53%) as an oil.

¹H NMR (300 MHz, CDCl₃) δ 1.90-1.99 (1H, m), 2.11-2.30 (5H, m), 2.55-2.67 (1H, m), 3.78-3.86 (2H, m), 4.79-4.90 (1H, m), 6.86-6.89 (1H, m), 7.10-7.13 (1H, m), 7.48-7.67 (2H, m), 7.73 (1H, s), 8.03-8.07 (1H, m), 10.36-10.39 (1H, m).

### Reference Example 189

### methyl 3-[(4-fluoro-2-nitrophenyl)amino]-2-methylbenzoate

A suspension of 1-bromo-4-fluoro-2-nitrobenzene (15 g, 68.2 mmol), methyl 3-amino-2-methylbenzoate (9.58 g, 58 mmol), tris(dibenzylideneacetone)dipalladium (0) (800 mg, 0.87 mmol), bis(2-diphenylphosphinophenyl)ether (942 mg, 1.75 mmol) and tripotassium phosphate (38.2 g, 180 mmol) in toluene (200 mL) was stirred at 105° C. overnight under an argon atmosphere. Water was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-85:15) to give the title compound (18.4 g, yield quant.) as a solid.

¹H NMR (300 MHz, CDCl₃) δ 2.46 (3H, s), 3.93 (3H, s), 6.68-6.72 (1H, m), 7.11-7.17 (1H, m), 7.29-7.32 (1H, m), 7.40-7.43 (1H, m), 7.80 (1H, d, J=7.8 Hz), 7.91-7.95 (1H, m), 9.20 (1H, s).

### Reference Example 190

### methyl 3-[(2-amino-4-fluorophenyl)amino]-2-methylbenzoate

A suspension of methyl 3-[(4-fluoro-2-nitrophenyl)amino]-2-methylbenzoate (18.4 g, 60.5 mmol) and palladium-carbon (0.5 g) in methanol (700 mL) was stirred overnight at room temperature under a hydrogen atmosphere. The reaction mixture was filtered and concentrated under reduced pressure to give the title compound (16.4 g, yield 99%) as a solid.

¹H NMR (300 MHz, CDCl₃) δ 2.46 (3H, s), 3.89 (5H, s), 5.03 (1H, s), 6.39-6.57 (3H, m), 6.88-6.93 (1H, m), 7.05 (1H, t, J=8.1 Hz), 7.23-7.26 (1H, m).

### Reference Example 191

### methyl 3-[(4-fluoro-2-{[(2R)-tetrahydrofuran-2-ylcarbonyl]amino}phenyl)amino]-2-methylbenzoate

A solution of methyl 3-[(2-amino-4-fluorophenyl)amino]-2-methylbenzoate (16.40 g, 59.8 mmol), (2R)-tetrahydrofuran-2-ylcarboxylic acid (6.97 g, 60.0 mmol), 1-ethyl-3-(3-dimethylaminopropyl)carbodiimide hydrochloride (13.8 g, 72 mmol) and 1-hydroxy-1H-benzotriazole monohydrate (11.0 g, 72 mmol) in acetonitrile (300 mL) was stirred overnight at room temperature. Water was added to the reaction mixture, and the mixture was extracted with ethyl acetate. The extract was washed with saturated brine, dried over anhydrous magnesium sulfate, and concentrated under reduced pressure. The residue was purified by basic silica gel column chromatography (hexane:ethyl acetate=90:10-70:30) to give the title compound (20.71 g, yield 93%) as an oil.

¹H NMR (300 MHz, CDCl₃) δ 1.43-1.55 (1H, m), 1.70-1.79 (1H, m), 1.94-2.04 (1H, m), 2.15-2.77 (1H, m), 2.52 (3H, s), 3.25-3.32 (1H, m), 3.61-3.69 (1H, m), 3.91 (3H, s), 4.34-4.38 (1H, m), 5.31 (1H, s), 6.49-6.51 (1H, m), 6.80-6.86 (1H, m), 7.00-7.10 (2H, m), 7.28-7.31 (1H, m), 8.06-8.11 (1H, m), 8.95 (1H, br s).

### Reference Example 192

### methyl 3-{5-fluoro-2-[(2R)-tetrahydrofuran-2-yl]-1H-benzimidazol-1-yl}-2-methylbenzoate

Methyl 3-[(4-fluoro-2-{[(2R)-tetrahydrofuran-2-ylcarbonyl]amino}phenyl)amino]-2-methylbenzoate (20.7 g, 55.6 mmol) and polyphosphoric acid (41 g) were stirred at 130° C. for min, and the reaction container was cooled to room temperature. Water was added to the reaction mixture, and the mixture was extracted with ethyl acetate. The extract was washed with saturated brine, dried over anhydrous magnesium sulfate, and concentrated under reduced pressure. The residue was purified by basic silica gel column chromatography (hexane:ethyl acetate=95:5-75:25) to give the title compound (11.5 g, yield 59%) as an oil.

¹H NMR (300 MHz, CDCl₃) δ 1.89-1.96 (1H, m), 2.04-2.23 (5H, m), 2.50-2.59 (1H, m), 3.77-3.91 (2H, m), 3.94 (3H, s), 4.73-4.86 (1H, m), 6.77-6.82 (1H, m), 6.94-7.00 (1H, m), 7.37-7.55 (3H, m), 8.04-8.07 (1H, m).

### Reference Example 193

### 3-{5-fluoro-2-[(2R)-tetrahydrofuran-2-yl]-1H-benzimidazol-1-yl}-2-methylbenzaldehyde

Lithium aluminum hydride (2.0 M tetrahydrofuran solution, 17 mL, 34 mmol) was added dropwise to a solution of methyl 3-{5-fluoro-2-[(2R)-tetrahydrofuran-2-yl]-1H-benzimidazol-1-yl}-2-methylbenzoate (11.54 g, 32.6 mmol) in tetrahydrofuran (250 mL) at 0° C., and the mixture was stirred at 0° C. for 1 hr. Sodium sulfate 10 hydrate was added to the reaction mixture at 0° C., and the mixture was stirred at room temperature for 1 hr. The reaction mixture was filtered through celite, and concentrated under reduced pressure to give an oil.

To a solution of the obtained oil in acetonitrile (300 mL) was added Dess-Martin periodonane (14.4 g, 34 mmol) at 0° C., and the mixture was stirred at 0° C. for 1 hr. An aqueous sodium thiosulfate solution and an aqueous sodium hydrogen carbonate solution were added to the reaction mixture, and the mixture was stirred at room temperature for 30 min. The reaction mixture was extracted with ethyl acetate, and the extract was washed with saturated brine, dried over anhydrous magnesium sulfate, and concentrated under reduced pressure. The residue was purified by silica gel column chromatography (hexane:ethyl acetate=90:10-60:40) to give the title compound (6.78 g, yield 64%, 2 steps) as an oil.

¹H NMR (300 MHz, CDCl₃) δ1.83-1.98 (1H, m), 2.00-2.29 (5H, m), 2.56-2.62 (1H, m), 3.72-3.85 (2H, m), 4.76-4.88 (1H, m), 6.77-6.83 (1H, m), 6.94-7.01 (1H, m), 7.47-7.65 (3H, m), 8.01-8.04 (1H, m), 10.35-10.39 (1H, m).

### Reference Example 194

### methyl 3-[(4-chloro-2-nitrophenyl)amino]-2-methylbenzoate

A suspension of 1-bromo-4-chloro-2-nitrobenzene (14.2 g, 60.0 mmol), methyl 3-amino-2-methylbenzoate (8.26 g, 50 mmol), tris(dibenzylideneacetone)dipalladium (0) (1.6 g, 1.74 mmol), bis(2-diphenylphosphinophenyl)ether (1.88 g, 3.48 mmol) and tripotassium phosphate (31.8 g, 150 mmol) in toluene (200 mL) was stirred at 105° C. overnight under an argon atmosphere. Water was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=90:10-50:50) to give the title compound (15.7 g, yield 98%) as a solid.

¹H NMR (300 MHz, CDCl₃) δ 2.45 (3H, s), 3.93 (3H, s), 6.65 (1H, d, J=9.3 Hz), 7.25-7.42 (3H, m), 7.80-7.83 (1H, m), 8.22 (1H, d, J=1.8 Hz), 9.29 (1H, br s).

### Reference Example 195

### methyl 3-[(4-chloro-2-{[(2R)-tetrahydrofuran-2-ylcarbonyl]amino}phenyl)amino]-2-methylbenzoate

A suspension of methyl 3-[(4-chloro-2-nitrophenyl)amino]-2-methylbenzoate (15.7 g, 49.0 mmol), reduced iron (13.96 g, 250.0 mmol), calcium chloride (2.77 g, 25.0 mmol) in ethanol (700 mL) and water (85 mL) was stirred at 95° C. for 7 hr. The reaction mixture was filtered and concentrated under reduced pressure to give a solid. The solid was washed with water and dried under reduced pressure to give a solid (14.0 g). A solution of the obtained solid (14.0 g), (2R)-tetrahydrofuran-2-ylcarboxylic acid (6.15 g, 53.0 mmol), 1-ethyl-3-(3-dimethylaminopropyl)carbodiimide hydrochloride (11.11 g, 58 mmol) and 1-hydroxy-1H-benzotriazole monohydrate (8.88 g, 58 mmol) in acetonitrile (200 mL) was stirred overnight at room temperature. Water was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=90:10-80:20) to give the title compound (17.1 g, yield 90%) as an oil.

¹H NMR (300 MHz, CDCl₃) δ1.54-1.63 (1H, m), 1.75-1.84 (1H, m), 1.95-2.06 (1H, m), 2.19-2.31 (1H, m), 2.48 (3H, s), 3.41-3.47 (1H, m), 3.67-3.75 (1H, m), 3.90 (3H, s), 4.36-4.41 (1H, m), 5.62 (1H, s), 6.67-6.70 (1H, m), 6.98-7.10 (3H, m), 7.33-7.36 (1H, m), 8.07 (1H, d, J=2.4 Hz), 8.78 (1H, s).

### Reference Example 196

### methyl 3-{5-chloro-2-[(2R)-tetrahydrofuran-2-yl]-1H-benzimidazol-1-yl}-2-methylbenzoate

Methyl 3-[(4-chloro-2-{[(2R)-tetrahydrofuran-2-ylcarbonyl]amino}phenyl)amino]-2-methylbenzoate (17.1 g, 44.0 mmol) and polyphosphoric acid (33 g) were stirred at 130° C. for min, and the reaction container was cooled to room temperature. Water was added to the reaction mixture, and the mixture was extracted with ethyl acetate. The extract was washed with saturated brine, dried over anhydrous magnesium sulfate, and concentrated under reduced pressure. The residue was purified by basic silica gel column chromatography (hexane:ethyl acetate=90:10-75:25) to give the title compound (13.2 g, yield 81%) as an oil.

¹H NMR (300 MHz, CDCl₃) δ 1.89-1.98 (1H, m), 2.10-2.24 (5H, m), 2.47-2.59 (1H, m), 3.76-3.91 (2H, m), 3.94 (3H, s), 4.74-4.87 (1H, m), 6.78-6.82 (1H, m), 7.16-7.20 (1H, m), 7.36-7.54 (2H, m), 7.80-7.81 (1H, m), 8.05-8.08 (1H, m).

### Reference Example 197

### 3-{5-chloro-2-[(2R)-tetrahydrofuran-2-yl]-1H-benzimidazol-1-yl}-2-methylbenzaldehyde

Lithium aluminum hydride (2.0 M tetrahydrofuran solution, mL, 40 mmol) was added dropwise to a solution of methyl 3-{5-chloro-2-[(2R)-tetrahydrofuran-2-yl]-1H-benzimidazol-1-yl}-2-methylbenzoate (13.2 g, 35.6 mmol) in tetrahydrofuran (200 mL) at 0° C., and the mixture was stirred at 0° C. for 0.5 hr. Sodium sulfate 10 hydrate was added to the reaction mixture at 0° C., and the mixture was stirred at room temperature for 1 hr. The reaction mixture was filtered through celite, and concentrated under reduced pressure to give a solid.

To a solution of the obtained solid in acetonitrile (200 mL) was added Dess-Martin periodonane (15.3 g, 36 mmol) at 0° C., and the mixture was stirred at 0° C. for 1 hr. An aqueous sodium thiosulfate solution and an aqueous sodium hydrogen carbonate solution were added to the reaction mixture, and the mixture was stirred at room temperature for 30 min. The reaction mixture was extracted with ethyl acetate, and the extract was washed with saturated brine, dried over anhydrous magnesium sulfate, and concentrated under reduced pressure. The residue was purified by silica gel column chromatography (hexane:ethyl acetate=90:10-75:25) to give the title compound (8.7 g, yield 72%, 2 steps) as a solid.

¹H NMR (300 MHz, CDCl₃) δ1.88-1.98 (1H, m), 2.09-2.27 (5H, m), 2.52-2.61 (1H, m), 3.76-3.84 (2H, m), 4.77-4.89 (1H, m), 6.78-6.81 (1H, m), 7.17-7.27 (1H, m), 7.47-7.65 (2H, m), 7.82-7.83 (1H, m), 8.01-8.05 (1H, m), 10.35-10.38 (1H, m).

### Example 1

### [(3S)-6-{[(3R)-7-bromo-2,3-dihydro-1-benzofuran-3-yl]amino}-2,3-dihydro-1-benzofuran-3-yl]acetic acid

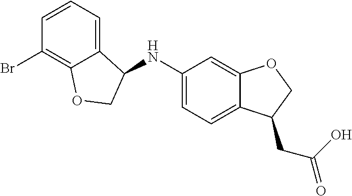

To a mixed solution of methyl [(3S)-6-{[(3R)-7-bromo-2,3-dihydro-1-benzofuran-3-yl](trifluoroacetyl)amino}-2,3-dihydro-1-benzofuran-3-yl]acetate (60.0 mg, 0.120 mmol) in tetrahydrofuran (1.0 mL) and methanol (0.5 mL) was added 1 M aqueous sodium hydroxide solution (360 μL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (48.8 mg, yield 100%) as a white solid.

¹H NMR (300 MHz, CDCl₃) δ 2.61 (1H, dd, J=17.0, 9.5 Hz), 2.80 (1H, dd, J=16.7, 5.3 Hz), 3.72-3.86 (1H, m), 4.27 (1H, dd, J=9.1, 6.1 Hz), 4.47 (1H, dd, J=9.5, 4.2 Hz), 4.68-4.84 (2H, m), 5.26 (1H, dd, J=7.2, 4.2 Hz), 6.10-6.19 (2H, m), 6.80 (1H, t, J=7.8 Hz), 7.00 (1H, d, J=8.0 Hz), 7.24-7.34 (1H, m), 7.40 (1H, d, J=8.0 Hz).

MS m/z 390 (M+H)⁺.

### Example 2

### [(3S)-6-{[(3S)-7-bromo-2,3-dihydro-1-benzofuran-3-yl]amino}2,3-dihydro-1-benzofuran-3-yl]acetic acid

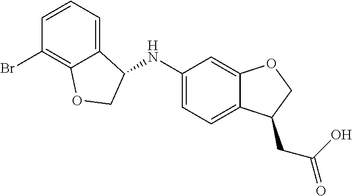

To a mixed solution of methyl [(3S)-6-{[(3S)-7-bromo-2,3-dihydro-1-benzofuran-3-yl](trifluoroacetyl)amino}-2,3-dihydro-1-benzofuran-3-yl]acetate (60.0 mg, 0.120 mmol) in tetrahydrofuran (1.0 mL) and methanol (0.5 mL) was added 1 M aqueous sodium hydroxide solution (360 μL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (52.3 mg, yield 100%) as a white solid.

¹H NMR (300 MHz, CDCl₃) δ 2.61 (1H, dd, J=17.0, 9.5 Hz), 2.80 (1H, dd, J=17.0, 5.3 Hz), 3.72-3.86 (1H, m), 4.27 (1H, dd, J=9.1, 6.1 Hz), 4.47 (1H, dd, J=9.8, 4.2 Hz), 4.69-4.85 (2H, m), 5.26 (1H, dd, J=7.4, 4.0 Hz), 6.09-6.17 (2H, m), 6.81 (1H, t, J=7.6 Hz), 7.00 (1H, d, J=8.7 Hz), 7.28 (1H, d, J=7.2 Hz), 7.41 (1H, d, J=8.0 Hz).

MS m/z 390 (M+H)⁺.

### Example 3

### [(3S)-6-{[(3S)-7-(2,5-dimethylthiophen-3-yl)-2,3-dihydro-1-benzofuran-3-yl]amino}-2,3-dihydro-1-benzofuran-3-yl]acetic acid

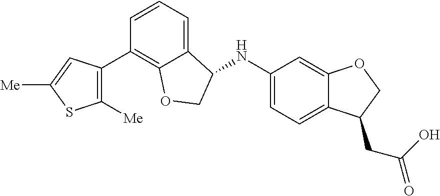

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (200 mg, 0.400 mmol), (2,5-dimethylthiophen-3-yl)boronic acid (74.8 mg, 0.480 mmol) and 2 M aqueous sodium carbonate solution (0.600 mL, 1.20 mmol) in toluene (2 mL) were added tris(dibenzylideneacetone)dipalladium (0) (14.7 mg, 0.016 mmol) and dicyclohexyl(2′,6′-dimethoxybiphenyl-2-yl)phosphane (26.3 mg, 0.064 mmol) under an argon atmosphere, and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give a colorless oil (230 mg). To a mixed solution of the obtained oil (230 mg) in tetrahydrofuran (2.7 mL) and methanol (1.35 mL) was added 1 M aqueous sodium hydroxide solution (1.30 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (114 mg, yield 61%) as a white solid.

¹H NMR (300 MHz, CDCl₃) δ 2.37 (3H, br s), 2.44 (3H, br s), 2.53-2.72 (1H, m), 2.73-2.91 (1H, m), 3.79 (1H, br s), 4.20-4.35 (1H, m), 4.35-4.50 (1H, m), 4.73 (2H, d, J=8.3 Hz), 5.20 (1H, br s), 6.14 (2H, br s), 6.74 (1H, br s), 6.86-7.11 (2H, m), 7.13-7.41 (2H, m).

MS m/z 422 (M+H)⁺.

### Example 4

### [(3S)-6-({(3S)-7-[(2R,6S)-2,6-dimethylmorpholin-4-yl]-2,3-dihydro-1-benzofuran-3-yl}amino)-2,3-dihydro-1-benzofuran-3-yl]acetic acid

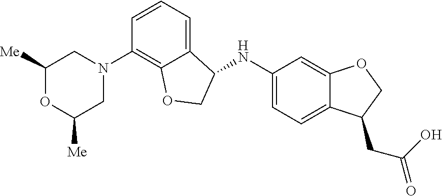

To a mixed solution of methyl {(3S)-6-[{(3S)-7-[(2R,6S)-2,6-dimethylmorpholin-4-yl]-2,3-dihydro-1-benzofuran-3-yl}(trifluoroacetyl)amino]-2,3-dihydro-1-benzofuran-3-yl}acetate (168 mg, 0.315 mmol) in tetrahydrofuran (2 mL) and methanol (1 mL) was added 1 M aqueous sodium hydroxide solution (945 μL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (89.7 mg, yield 67%) as a pale-yellow solid.

¹H NMR (300 MHz, CDCl₃) δ 1.19-1.27 (6H, m), 2.38 (2H, td, J=10.9, 4.0 Hz), 2.60 (1H, dd, J=16.7, 9.5 Hz), 2.79 (1H, dd, J=16.7, 5.3 Hz), 3.40-3.54 (2H, m), 3.71-3.96 (3H, m), 4.26 (1H, dd, J=9.1, 6.1 Hz), 4.44 (1H, dd, J=9.5, 3.8 Hz), 4.67-4.78 (2H, m), 5.13 (1H, dd, J=7.2, 3.8 Hz), 6.07-6.16 (2H, m), 6.76-6.82 (1H, m), 6.88 (1H, t, J=7.8 Hz), 6.95-7.03 (2H, m).

MS m/z 425 (M+H)⁺.

### Example 5

### [(3S)-6-{[(3S)-7-(piperidin-1-yl)-2,3-dihydro-1-benzofuran-3-yl]amino}-2,3-dihydro-1-benzofuran-3-yl]acetic acid

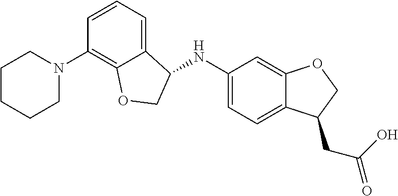

To a mixed solution of methyl [(3S)-6-{[(3S)-7-(piperidin-1-yl)-2,3-dihydro-1-benzofuran-3-yl](trifluoroacetyl)amino}-2,3-dihydro-1-benzofuran-3-yl]acetate (67.5 mg, 0.134 mmol) in tetrahydrofuran (1mL) and methanol (0.5 mL) was added 1 M aqueous sodium hydroxide solution (401 μL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (32.2 mg, yield 61%) as a pale-yellow solid.

¹H NMR (300 MHz, CDCl₃) δ 1.51-1.63 (2H, m), 1.68-1.83 (4H, m), 2.59 (1H, dd, J=16.7, 9.1 Hz), 2.78 (1H, dd, J=16.7, 5.3 Hz), 2.99-3.17 (4H, m), 3.70-3.85 (1H, m), 4.25 (1H, dd, J=9.3, 6.2 Hz), 4.43 (1H, dd, J=9.5, 3.8 Hz), 4.64-4.79 (2H, m), 5.13 (1H, dd, J=7.2, 3.8 Hz), 6.05-6.15 (2H, m), 6.78-6.92 (2H, m), 6.97 (2H, d, J=8.3 Hz).

MS m/z 395 (M+H)⁺.

### Example 6

### [(3S)-6-{[(3R)-7-{2,6-dimethyl-4-[3-(methylsulfonyl)propoxy]phenyl}-2,3-dihydro-1-benzofuran-3-yl]amino}-2,3-dihydro-1-benzofuran-3-yl]acetic acid

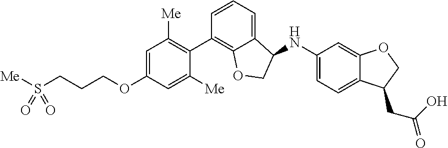

To a solution of methyl [(3S)-6-{[(3R)-7-bromo-2,3-dihydro-1-benzofuran-3-yl](trifluoroacetyl)amino}-2,3-dihydro-1-benzofuran-3-yl]acetate (200 mg, 0.400 mmol), {2,6-dimethyl-4-[3-(methylthio)propoxy]phenyl}boronic acid (229 mg, 0.800 mmol) synthesized according to WO2008/001931 and 2 M aqueous sodium carbonate solution (0.600 mL, 1.2 mmol) in toluene (1.3 mL) were added tris(dibenzylideneacetone)dipalladium (0) (14.7 mg, 0.016 mmol) and dicyclohexyl(2′,6′-dimethoxybiphenyl-2-yl)phosphane (26.3 mg, 0.064 mmol) under an argon atmosphere, and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give a pale-yellow oil (209 mg). To a solution of the obtained oil (209 mg) in ethyl acetate (1.7 mL) was added m-chloroperbenzoic acid (65%, 120 mg, 0.453 mmol) under ice-cooling, and the mixture was stirred at 0° C. for min. Then, saturated aqueous sodium hydrogen carbonate was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) to give a colorless oil (157 mg). To a mixed solution of the obtained oil (157 mg) in tetrahydrofuran (1.5 mL) and methanol (0.75 mL) was added 1 M aqueous sodium hydroxide solution (714 μL), and the mixture was stirred at room temperature for 3 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (111 mg, yield 50%) as a white solid.

¹H NMR (300 MHz, CDCl₃) δ 2.05 (3H, s), 2.08 (3H, s), 2.27-2.41 (2H, m), 2.61 (1H, dd, J=16.7, 9.1 Hz), 2.80 (1H, dd, J=16.7, 5.3 Hz), 2.96 (3H, s), 3.20-3.30 (2H, m), 3.72-3.86 (1H, m), 4.11 (2H, t, J=5.7 Hz), 4.22-4.38 (2H, m), 4.67 (1H, dd, J=9.7, 7.4 Hz), 4.74 (1H, t, J=9.1 Hz), 5.22 (1H, dd, J=7.2, 4.2 Hz), 6.10-6.20 (2H, m), 6.66 (2H, s), 6.94-7.06 (3H, m), 7.34 (1H, dd, J=6.8, 1.5 Hz).

MS m/z 552 (M+H)⁺.

### Example 7

### [(3S)-6-{[(3S)-7-{2,6-dimethyl-4-[3-(methylsulfonyl)propoxy]phenyl}-2,3-dihydro-1-benzofuran-3-yl]amino}-2,3-dihydro-1-benzofuran-3-yl]acetic acid

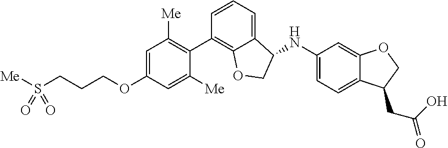

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (2.00 g, 4.00 mmol), {2,6-dimethyl-4-[3-(methylthio)propoxy]phenyl}boronic acid (1.72 g, 6.00 mmol) and 2 M aqueous sodium carbonate solution (6.00 mL, 12.0 mmol) in toluene (13 mL) were added tris(dibenzylideneacetone)dipalladium (0) (147 mg, 0.160 mmol) and dicyclohexyl(2′,6′-dimethoxybiphenyl-2-yl)phosphane (263 mg, 0.640 mmol) under an argon atmosphere, and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give a yellow oil (2.75 g). To a solution of the oil (2.75 g) obtained above in ethyl acetate (20 mL) was added m-chloroperbenzoic acid (65%, 1.45 g, 5.46 mmol) under ice-cooling, and the mixture was stirred at 0° C. for 1 hr. Then, saturated aqueous sodium hydrogen carbonate was added to the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-40:60) to give a yellow solid (1.59 g). To a mixed solution of the obtained yellow solid (1.59 g) in tetrahydrofuran (15 mL) and methanol (7.5 mL) was added 1 M aqueous sodium hydroxide solution (7.22 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a pale-yellow solid. This was recrystallized from ethyl acetate-heptane to give the title compound (1.16 g, yield 52%) as white crystals.

¹H NMR (300 MHz, CDCl₃) δ 2.05 (3H, s), 2.08 (3H, s), 2.26-2.43 (2H, m), 2.61 (1H, dd, J=16.6, 9.0 Hz), 2.81 (1H, dd, J=17.0, 5.3 Hz), 2.96 (3H, s), 3.19-3.33 (2H, m), 3.73-3.88 (1H, m), 4.11 (2H, t, J=5.8 Hz), 4.23-4.41 (2H, m), 4.63-4.83 (2H, m), 5.22 (1H, dd, J=7.2, 4.5 Hz), 6.10-6.23 (2H, m), 6.66 (2H, s), 6.94-7.09 (3H, m), 7.32-7.43 (1H, m).

MS m/z 552 (M+H)⁺.

elemental analysis value for C₃₀H₃₃NO₇S

Calculated: C, 65.32; H, 6.03; N, 2.54.

Found: C, 65.13; H, 5.98; N, 2.29.

melting point 169° C.

### Example 8

### [(3S)-6-{[(3S)-7-(2,6-dimethyl-4-{[(2S)-5-oxopyrrolidin-2-yl]methoxy}phenyl)-2,3-dihydro-1-benzofuran-3-yl]amino}-2,3-dihydro-1-benzofuran-3-yl]acetic acid

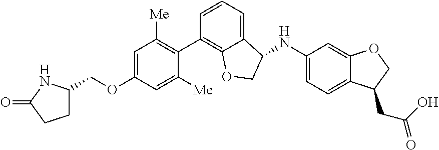

To a solution of methyl [(3S)-6-{[(3S)-7-(2,6-dimethyl-4-{[(2S)-5-oxopyrrolidin-2-yl]methoxy}phenyl)-2,3-dihydro-1-benzofuran-3-yl](trifluoroacetyl)amino}-2,3-dihydro-1-benzofuran-3-yl]acetate (2.92 g, 4.58 mmol) in methanol (23 mL) was added potassium carbonate (1.90 g, 13.7 mmol), and the mixture was stirred at 50° C. for 3 hr. The reaction mixture was filtered and concentrated under reduced pressure to give an oil compound. This was dissolved in tetrahydrofuran (30 mL) and methanol (15 mL), 1 M aqueous sodium hydroxide solution (13.7 mL) was added, and the mixture was stirred at 50° C. for 2 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a white solid. This was recrystallized from ethanol-water to give the title compound (1.63 g, yield 67%) as a pale-gray solid.

¹H NMR (300 MHz, CDCl₃) δ 1.81-2.13 (7H, m), 2.22-2.64 (4H, m), 2.78 (1H, dd, J=16.7, 5.3 Hz), 3.68-3.90 (2H, m), 3.94-4.18 (2H, m), 4.19-4.37 (2H, m), 4.64 (1H, dd, J=9.8, 7.2 Hz), 4.75 (1H, t, J=8.9 Hz), 5.11-5.26 (1H, m), 6.02-6.16 (2H, m), 6.59-6.74 (2H, m), 6.89-7.06 (3H, m), 7.19-7.38 (2H, m).

MS m/z 529 (M+H)⁺.

elemental analysis value for C₃₁H₃₂N₂O₆.

Calculated: C, 70.44; H, 6.10; N, 5.30.

Found: C, 70.34; H, 6.12; N, 5.30.

melting point 161° C.

### Example 9

### [(3S)-6-{[(3S)-7-{4-[(1,1-dioxidotetrahydro-2H-thiopyran-4-yl)oxy]-2,6-dimethylphenyl}-2,3-dihydro-1-benzofuran-3-yl]amino}-2,3-dihydro-1-benzofuran-3-yl]acetic acid

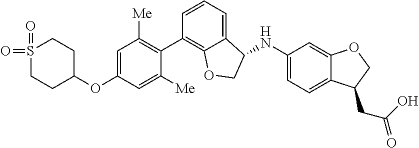

To a solution of methyl [(3S)-6-{[(3S)-7-bromo-2,3-dihydro-1-benzofuran-3-yl](trifluoroacetyl)amino}-2,3-dihydro-1-benzofuran-3-yl]acetate (6.00 g, 12.0 mmol), [4-(methoxymethoxy)-2,6-dimethylphenyl]boronic acid (3.02 g, 14.4 mmol) and 2 M aqueous sodium carbonate solution (18.0 mL, 36.0 mmol) in toluene (40 mL) were added tris(dibenzylideneacetone)dipalladium (0) (439 mg, 0.480 mmol) and dicyclohexyl(2′,6′-dimethoxybiphenyl-2-yl)phosphane (788 mg, 1.92 mmol) under an argon atmosphere, and the mixture was stirred at 100° C. overnight. The reaction mixture was cooled to room temperature, filtered through celite, and concentrated under reduced pressure. The residue was purified by silica gel column chromatography (hexane:ethyl acetate=95:5-70:30) to give a yellow oil (7.65 g). To a solution of the obtained oil (7.65 g) in methanol (40 mL) was added 10% hydrogen chloride containing methanol solution (3.8 mL), and the mixture was stirred at 40° C. for 2 hr. The reaction mixture was neutralized with saturated aqueous sodium hydrogen carbonate, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give a white solid (6.47 g). To a solution of the obtained solid (2.00 g), tetrahydro-2H-thiopyran-4-ol (480 mg, 4.06 mmol) and triphenylphosphine (1.26 g, 4.81 mmol) in tetrahydrofuran (19 mL) was added diethyl azodicarboxylate (40% toluene solution, 2.19 mL, 4.81 mmol). The mixture was stirred at room temperature for 2 hr, diethyl azodicarboxylate (2.19 mL) and triphenylphosphine (1.26 g) were added, and the mixture was further stirred for 1 hr. Diethyl azodicarboxylate (2.19 mL) and triphenylphosphine (1.26 g) were further added, and the mixture was stirred for 10 min and concentrated under reduced pressure. The residue was purified by silica gel column chromatography (hexane:ethyl acetate=95:5-70:30) to give a white solid (1.86 g). To a solution of the obtained solid (1.86 g) in ethyl acetate (15 mL) was added m-chloroperbenzoic acid (65%, 1.50 g, 5.65 mmol) under ice-cooling, and the mixture was stirred at room temperature for 3 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-30:70) to give a white solid (1.57 g). To a mixed solution of the obtained solid (1.57 g) in tetrahydrofuran (15 mL) and methanol (7.5 mL) was added 1 M aqueous sodium hydroxide solution (7.01 mL), and the mixture was stirred at 50° C. for 2 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a white solid. This was triturated with hexane-ethyl acetate, and recrystallized from ethyl acetate-heptane to give the title compound (911 mg, yield 44%) as a white solid.

¹H NMR (300 MHz, CDCl₃) δ 2.07 (6H, d, J=9.0 Hz), 2.27-2.71 (5H, m), 2.81 (1H, dd, J=16.6, 5.3 Hz), 2.88-3.04 (2H, m), 3.35-3.55 (2H, m), 3.74-3.88 (1H, m), 4.23-4.42 (2H, m), 4.62-4.83 (3H, m), 5.17-5.31 (1H, m), 6.09-6.21 (2H, m), 6.69 (2H, s), 6.95-7.11 (3H, m), 7.32-7.44 (1H, m).

MS m/z 564 (M+H)⁺.

melting point 176° C.

### Example 10

### [(3S)-6-{[(3R)-7-(2-methyl-1H-benzimidazol-1-yl)-2,3-dihydro-1-benzofuran-3-yl]amino}-2,3-dihydro-1-benzofuran-3-yl]acetic acid

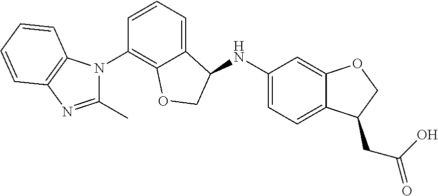

To a mixed solution of methyl [(3S)-6-{[(3R)-7-(2-methyl-1H-benzimidazol-1-yl)-2,3-dihydro-1-benzofuran-3-yl](trifluoroacetyl)amino}-2,3-dihydro-1-benzofuran-3-yl]acetate (684 mg, 1.24 mmol) in tetrahydrofuran (7.8 mL) and methanol (3.9 mL) was added 1 M aqueous sodium hydroxide solution (3.72 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (485 mg, yield 89%) as a white solid.

¹H NMR (300 MHz, DMSO-d₆) δ 2.38-2.54 (4H, m), 2.60-2.77 (1H, m), 3.52-3.67 (1H, m), 4.13 (1H, dd, J=8.7, 6.8 Hz), 4.21-4.31 (1H, m), 4.57-4.67 (1H, m), 4.76-4.88 (1H, m), 5.34-5.45 (1H, m), 6.13-6.27 (3H, m), 6.97 (1H, d, J=7.9 Hz), 7.06-7.33 (4H, m), 7.45 (1H, d, J=7.9 Hz), 7.55 (1H, d, J=7.5 Hz), 7.65 (1H, d, J=6.8 Hz), 12.30 (1H, br s).

MS m/z 442 (M+H)⁺.

### Example 11

### [(3S)-6-{[(3R)-7-(2-ethyl-1H-benzimidazol-1-yl)-2,3-dihydro-1-benzofuran-3-yl]amino}-2,3-dihydro-1-benzofuran-3-yl]acetic acid

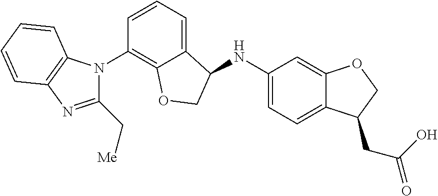

To a mixed solution of methyl [(3S)-6-{[(3R)-7-(2-ethyl-1H-benzimidazol-1-yl)-2,3-dihydro-1-benzofuran-3-yl](trifluoroacetyl)amino}-2,3-dihydro-1-benzofuran-3-yl]acetate (231 mg, 0.408 mmol) in tetrahydrofuran (2.6 mL) and methanol (1.3 mL) was added 1 M aqueous sodium hydroxide solution (1.22 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (159 mg, yield 86%) as a white solid.

¹H NMR (300 MHz, DMSO-d₆) δ 1.25 (3H, q, J=7.5 Hz), 2.39-2.47 (1H, m), 2.60-2.77 (3H, m), 3.54-3.68 (1H, m), 4.12 (1H, dd, J=9.0, 6.8 Hz), 4.18-4.29 (1H, m), 4.62 (1H, t, J=9.0 Hz), 4.73-4.84 (1H, m), 5.33-5.45 (1H, m), 6.15-6.27 (3H, m), 6.92-7.26 (5H, m), 7.41 (1H, d, J=7.9 Hz), 7.53 (1H, d, J=7.5 Hz), 7.61-7.67 (1H, m), 12.31 (1H, br s).

elemental analysis value for C₂₇H₂₅N₃O₄

Calculated: C, 71.19; H, 5.53; N, 9.22.

Found: C, 71.32; H, 5.65; N, 9.07.

MS m/z 456 (M+H)⁺.

### Example 12

### [(3S)-6-{[(3R)-7-(2-ethoxy-1H-benzimidazol-1-yl)-2,3-dihydro-1-benzofuran-3-yl]amino}-2,3-dihydro-1-benzofuran-3-yl]acetic acid

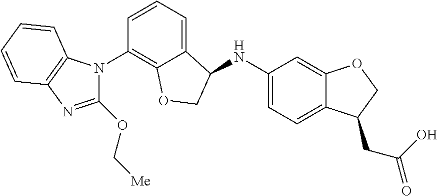

To a mixed solution of methyl [(3S)-6-{[(3R)-7-(2-ethoxy-1H-benzimidazol-1-yl)-2,3-dihydro-1-benzofuran-3-yl](trifluoroacetyl)amino}-2,3-dihydro-1-benzofuran-3-yl]acetate (284 mg, 0.489 mmol) in tetrahydrofuran (3.0 mL) and methanol (1.5 mL) was added 1 M aqueous sodium hydroxide solution (1.47 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (207 mg, yield 90%) as a white solid.

¹H NMR (300 MHz, DMSO-d₆) δ 1.35 (3H, t, J=7.0 Hz), 2.38-2.47 (1H, m), 2.65 (1H, dd, J=16.6, 5.7 Hz), 3.55-3.68 (1H, m), 4.12 (1H, dd, J=8.9, 6.6 Hz), 4.18-4.28 (1H, m), 4.54 (2H, q, J=7.2 Hz), 4.62 (1H, t, J=9.0 Hz), 4.71-4.90 (1H, m), 5.32-5.43 (1H, m), 6.16-6.29 (3H, m), 6.90-7.03 (2H, m), 7.04-7.19 (3H, m), 7.38 (1H, d, J=7.5 Hz), 7.47 (2H, d, J=7.2 Hz), 12.33 (1H, br s).

MS m/z 472 (M+H)⁺.

### Example 13

### [(3S)-6-{[(3R)-7-(2-ethyl-6-fluoro-1H-benzimidazol-1-yl)-2,3-dihydro-1-benzofuran-3-yl]amino}-2,3-dihydro-1-benzofuran-3-yl]acetic acid

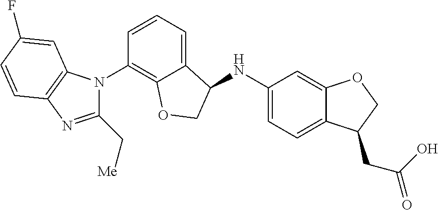

To a mixed solution of methyl [(3S)-6-{[(3R)-7-(2-ethyl-6-fluoro-1H-benzimidazol-1-yl)-2,3-dihydro-1-benzofuran-3-yl](trifluoroacetyl)amino}-2,3-dihydro-1-benzofuran-3-yl]acetate (249 mg, 0.427 mmol) in tetrahydrofuran (2.6 mL) and methanol (1.3 mL) was added 1 M aqueous sodium hydroxide solution (1.28 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (172 mg, yield 85%) as a white solid.

¹H NMR (300 MHz, DMSO-d₆) δ 1.24 (3H, q, J=7.5 Hz), 2.39-2.49 (1H, m), 2.59-2.77 (3H, m), 3.55-3.69 (1H, m), 4.07-4.17 (1H, m), 4.20-4.29 (1H, m), 4.62 (1H, td, J=8.9, 1.5 Hz), 4.72-4.86 (1H, m), 5.33-5.44 (1H, m), 6.13-6.26 (3H, m), 6.79-6.90 (1H, m), 6.97 (1H, dd, J=7.9, 2.6 Hz), 7.01-7.17 (2H, m), 7.43 (1H, d, J=7.9 Hz), 7.54 (1H, d, J=7.5 Hz), 7.65 (1H, dd, J=8.7, 4.9 Hz), 12.31 (1H, br s).

MS m/z 474 (M+H)⁺.

### Example 14

### [(3S)-6-{[(3R)-7-(2-ethoxy-6-fluoro-1H-benzimidazol-1-yl)-2,3-dihydro-1-benzofuran-3-yl]amino}-2,3-dihydro-1-benzofuran-3-yl]acetic acid

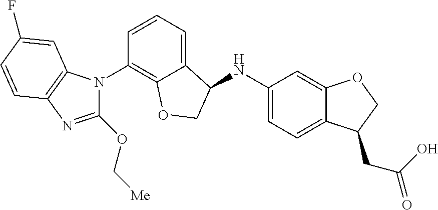

To a mixed solution of methyl [(3S)-6-{[(3R)-7-(2-ethoxy-6-fluoro-1H-benzimidazol-1-yl)-2,3-dihydro-1-benzofuran-3-yl](trifluoroacetyl)amino}-2,3-dihydro-1-benzofuran-3-yl]acetate (281 mg, 0.468 mmol) in tetrahydrofuran (3.0 mL) and methanol (1.5 mL) was added 1 M aqueous sodium hydroxide solution (1.40 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (193 mg, yield 84%) as a white solid.

¹H NMR (300 MHz, DMSO-d₆) δ 1.34 (3H, t, J=7.2 Hz), 2.38-2.49 (1H, m), 2.66 (1H, dd, J=16.6, 5.7 Hz), 3.54-3.69 (1H, m), 4.12 (1H, dd, J=8.7, 6.8 Hz), 4.18-4.35 (1H, m), 4.53 (2H, q, J=6.9 Hz), 4.62 (1H, t, J=9.0 Hz), 4.70-4.97 (1H, m), 5.31-5.43 (1H, m), 6.13-6.31 (3H, m), 6.74-6.90 (1H, m), 6.93-7.03 (2H, m), 7.08 (1H, t, J=7.7 Hz), 7.39 (1H, d, J=7.9 Hz), 7.43-7.52 (2H, m), 12.32 (1H, br s).

MS m/z 490 (M+H)⁺.

### Example 15

### [(3S)-6-{[(3S)-7-(2-methyl-1H-benzimidazol-1-yl)-2,3-dihydro-1-benzofuran-3-yl]amino}-2,3-dihydro-1-benzofuran-3-yl]acetic acid

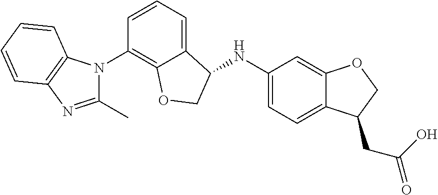

To a mixed solution of methyl [(3S)-6-{[(3S)-7-(2-methyl-1H-benzimidazol-1-yl)-2,3-dihydro-1-benzofuran-3-yl](trifluoroacetyl)amino}-2,3-dihydro-1-benzofuran-3-yl]acetate (647 mg, 1.17 mmol) in tetrahydrofuran (7.3 mL) and methanol (3.7 mL) was added 1 M aqueous sodium hydroxide solution (3.51 mL), and the mixture was stirred at 50° C. for 1 hr. 1 M Hydrochloric acid and 10% aqueous citric acid solution were added to the reaction mixture. The mixture was neutralized with saturated aqueous sodium hydrogen carbonate, diluted with distilled water, and extracted with ethyl acetate. The extract was washed with saturated brine, dried over anhydrous magnesium sulfate, and concentrated under reduced pressure to give the title compound (369 mg, yield 71%) as a white solid.

¹H NMR (300 MHz, DMSO-d₆) δ 2.36-2.46 (4H, m), 2.66 (1H, dd, J=16.2, 5.3 Hz), 3.52-3.70 (1H, m), 4.07-4.19 (1H, m), 4.19-4.32 (1H, m), 4.63 (1H, t, J=8.9 Hz), 4.74-4.88 (1H, m), 5.31-5.47 (1H, m), 6.11-6.28 (3H, m), 6.91-7.28 (5H, m), 7.42 (1H, d, J=7.9 Hz), 7.53 (1H, d, J=7.5 Hz), 7.57-7.66 (1H, m), 12.32 (1H, br s).

MS m/z 442 (M+H)⁺.

### Example 16

### sodium [(3S)-6-{[(3S)-7-(2-methyl-1H-benzimidazol-1-yl)-2,3-dihydro-1-benzofuran-3-yl]amino}-2,3-dihydro-1-benzofuran-3-yl]acetate

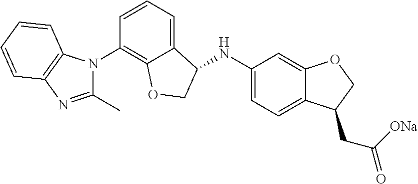

To a suspension of [(3S)-6-{[(3S)-7-(2-methyl-1H-benzimidazol-1-yl)-2,3-dihydro-1-benzofuran-3-yl]amino}-2,3-dihydro-1-benzofuran-3-yl]acetic acid (369 mg, 0.836 mmol) in water (4 mL) was added 1 M aqueous sodium hydroxide solution (0.836 mL), and the mixture was stirred at room temperature. After dissolution of insoluble material, acetonitrile was added and the mixture was concentrated under reduced pressure to give the title compound (286 mg, yield 74%) as a pale-brown solid.

¹H NMR (300 MHz, DMSO-d₆) δ 2.01 (1H, dd, J=15.1, 10.2 Hz), 2.29-2.45 (4H, m), 3.49-3.65 (1H, m), 4.08 (1H, t, J=7.9 Hz), 4.19-4.32 (1H, m), 4.62 (1H, t, J=9.0 Hz), 4.80 (1H, q, J=7.9 Hz), 5.32-5.44 (1H, m), 6.04-6.23 (3H, m), 6.94 (1H, d, J=7.9 Hz), 7.00-7.27 (4H, m), 7.41 (1H, d, J=7.5 Hz), 7.52 (1H, d, J=7.2 Hz), 7.58-7.67 (1H, m).

MS m/z 442 (M+H)⁺ (as free form).

### Example 17

### sodium [(3S)-6-{[(3S)-7-(6-fluoro-2-methyl-1H-benzimidazol-1-yl)-2,3-dihydro-1-benzofuran-3-yl]amino}-2,3-dihydro-1-benzofuran-3-yl]acetate

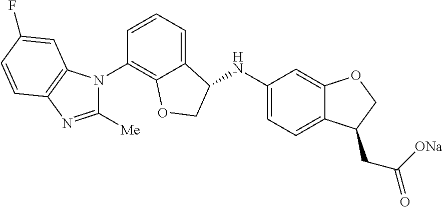

To a suspension of [(3S)-6-{[(3S)-7-(6-fluoro-2-methyl-1H-benzimidazol-1-yl)-2,3-dihydro-1-benzofuran-3-yl]amino}-2,3-dihydro-1-benzofuran-3-yl]acetic acid (100 mg, 0.218 mmol) in water (1.1 mL) was added 1 M aqueous sodium hydroxide solution (0.218 mL), and the mixture was stirred at room temperature. After dissolution of insoluble material, acetonitrile was added and the mixture was concentrated under reduced pressure to give a solid. This was triturated with hexane-ethyl acetate to give the title compound (96.8 mg, yield 92%) as a white solid.

¹H NMR (300 MHz, DMSO-d₆) δ 1.98 (1H, dd, J=14.9, 10.0 Hz), 2.32 (1H, dd, J=15.1, 4.9 Hz), 2.40 (3H, d, J=5.7 Hz), 3.47-3.65 (1H, m), 4.03-4.13 (1H, m), 4.26 (1H, dd, J=9.4, 4.5 Hz), 4.61 (1H, t, J=8.9 Hz), 4.73-4.87 (1H, m), 5.30-5.44 (1H, m), 6.08 (1H, dd, J=8.1, 6.6 Hz), 6.12-6.21 (2H, m), 6.81-6.98 (2H, m), 7.00-7.17 (2H, m), 7.43 (1H, d, J=7.9 Hz), 7.53 (1H, d, J=7.2 Hz), 7.61 (1H, dd, J=8.7, 4.9 Hz).

MS m/z 460 (M+H)⁺ (as free form).

### Example 18

### [(3S)-6-{[(3S)-7-(2-ethyl-1H-benzimidazol-1-yl)-2,3-dihydro-1-benzofuran-3-yl]amino}-2,3-dihydro-1-benzofuran-3-yl]acetic acid

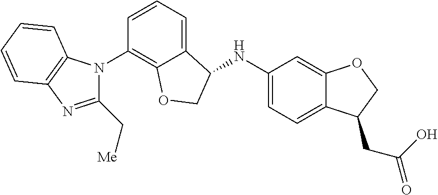

To a mixed solution of methyl [(3S)-6-{[(3S)-7-(2-ethyl-1H-benzimidazol-1-yl)-2,3-dihydro-1-benzofuran-3-yl](trifluoroacetyl)amino}-2,3-dihydro-1-benzofuran-3-yl]acetate (0.303 mmol) in tetrahydrofuran (2.0 mL) and methanol (1.0 mL) was added 1 M aqueous sodium hydroxide solution (0.909 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (77.7 mg, yield 56%) as a white solid.

¹H NMR (300 MHz, DMSO-d₆) δ 1.25 (3H, q, J=7.5 Hz), 2.37-2.47 (1H, m), 2.59-2.79 (3H, m), 3.55-3.69 (1H, m), 4.08-4.17 (1H, m), 4.18-4.28 (1H, m), 4.63 (1H, t, J=8.9 Hz), 4.74-4.85 (1H, m), 5.32-5.46 (1H, m), 6.14-6.28 (3H, m), 6.92-7.26 (5H, m), 7.41 (1H, d, J=7.9 Hz), 7.53 (1H, d, J=7.5 Hz), 7.60-7.68 (1H, m), 12.31 (1H, br s).

MS m/z 456 (M+H)⁺.

### Example 19

### sodium [(3S)-6-{[(3S)-7-(2-ethyl-1H-benzimidazol-1-yl)-2,3-dihydro-1-benzofuran-3-yl]amino}-2,3-dihydro-1-benzofuran-3-yl]acetate

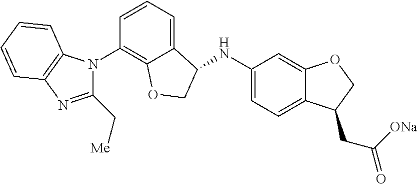

To a suspension of [(3S)-6-{[(3S)-7-(2-ethyl-1H-benzimidazol-1-yl)-2,3-dihydro-1-benzofuran-3-yl]amino}-2,3-dihydro-1-benzofuran-3-yl]acetic acid (77.7 mg, 0.171 mmol) in water (1.0 mL) was added 1 M aqueous sodium hydroxide solution (0.171 mL), and the mixture was stirred at room temperature. After dissolution of insoluble material, acetonitrile was added and the mixture was concentrated under reduced pressure to give a solid. This was triturated with acetonitrile to give the title compound (61.9 mg, yield 76%) as a white solid.

¹H NMR (300 MHz, DMSO-d₆) δ 1.25 (3H, q, J=7.8 Hz), 1.85-1.99 (1H, m), 2.20-2.33 (1H, m), 2.65-2.78 (2H, m), 3.43-3.57 (1H, m), 4.01-4.11 (1H, m), 4.18-4.29 (1H, m), 4.59 (1H, t, J=8.9 Hz), 4.73-4.85 (1H, m), 5.32-5.44 (1H, m), 6.02-6.21 (3H, m), 6.91 (1H, d, J=8.0 Hz), 6.98-7.28 (4H, m), 7.40 (1H, d, J=7.2 Hz), 7.53 (1H, d, J=8.0 Hz), 7.64 (1H, d, J=7.6 Hz).

MS m/z 456 (M+H)⁺ (as free form).

### Example 20

### [(3S)-6-{[(3S)-7-(2-ethoxy-1H-benzimidazol-1-yl)-2,3-dihydro-1-benzofuran-3-yl]amino}-2,3-dihydro-1-benzofuran-3-yl]acetic acid

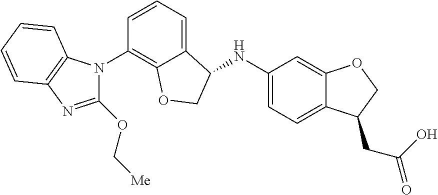

To a mixed solution of methyl [(3S)-6-{[(3S)-7-(2-ethoxy-1H-benzimidazol-1-yl) 2,3-dihydro-1-benzofuran-3-yl](trifluoroacetyl)amino}-2,3-dihydro-1-benzofuran-3-yl]acetate (163 mg, 0.280 mmol) in tetrahydrofuran (1.8 mL) and methanol (0.9 mL) was added 1 M aqueous sodium hydroxide solution (0.839 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (90.6 mg, yield 69%) as a white solid.

¹H NMR (300 MHz, DMSO-d₆) δ 1.35 (3H, t, J=7.2 Hz), 2.37-2.47 (1H, m), 2.65 (1H, dd, J=16.2, 5.7 Hz), 3.55-3.67 (1H, m), 4.12 (1H, dd, J=8.7, 6.8 Hz), 4.18-4.27 (1H, m), 4.54 (2H, q, J=7.0 Hz), 4.62 (1H, t, J=8.9 Hz), 4.74-4.88 (1H, m), 5.33-5.42 (1H, m), 6.16-6.26 (3H, m), 6.96 (2H, d, J=7.9 Hz), 7.04-7.18 (3H, m), 7.38 (1H, d, J=7.9 Hz), 7.47 (2H, d, J=7.5 Hz), 12.34 (1H, br s).

MS m/z 472 (M+H)⁺.

### Example 21

### sodium [(3S)-6-{[(3S)-7-(2-ethoxy-1H-benzimidazol-1-yl)-2,3-dihydro-1-benzofuran-3-yl]amino}-2,3-dihydro-1-benzofuran-3-yl]acetate

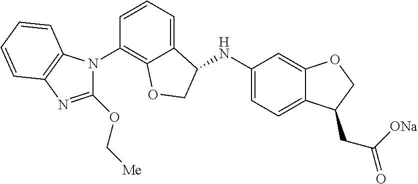

To a suspension of [(3S)-6-{[(3S)-7-(2-ethoxy-1H-benzimidazol-1-yl)-2,3-dihydro-1-benzofuran-3-yl]amino}-2,3-dihydro-1-benzofuran-3-yl]acetic acid (90.6 mg, 0.192 mmol) in water (1.0 mL) was added 1 M aqueous sodium hydroxide solution (0.192 mL), and the mixture was stirred at room temperature. After dissolution of insoluble material, acetonitrile was added and the mixture was concentrated under reduced pressure to give a solid. This was triturated with acetonitrile to give the title compound (93.5 mg, yield 99%) as a white solid.

¹H NMR (300 MHz, DMSO-d₆) δ 1.30-1.40 (3H, m), 1.91-2.09 (1H, m), 2.32 (1H, dd, J=15.0, 4.7 Hz), 3.47-3.63 (1H, m), 4.07 (1H, t, J=8.0 Hz), 4.17-4.30 (1H, m), 4.47-4.67 (3H, m), 4.70-4.89 (1H, m), 5.28-5.43 (1H, m), 6.01-6.23 (3H, m), 6.84-7.20 (5H, m), 7.37 (1H, d, J=8.0 Hz), 7.47 (2H, d, J=7.6 Hz).

MS m/z 472 (M+H)⁺ (as free form).

### Example 22

### [(3S)-6-{[(3S)-7-(2-ethyl-6-fluoro-1H-benzimidazol-1-yl)-2,3-dihydro-1-benzofuran-3-yl]amino}-2,3-dihydro-1-benzofuran-3-yl]acetic acid

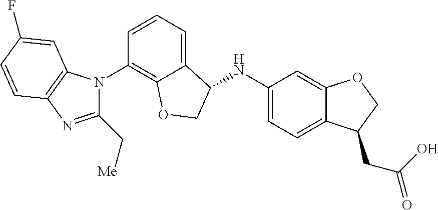

To a mixed solution of methyl [(3S)-6-{[(3S)-7-(2-ethyl-6-fluoro-1H-benzimidazol-1-yl)-2,3-dihydro-1-benzofuran-3-yl](trifluoroacetyl)amino}-2,3-dihydro-1-benzofuran-3-yl]acetate (147 mg, 0.253 mmol) in tetrahydrofuran (1.6 mL) and methanol (0.8 mL) was added 1 M aqueous sodium hydroxide solution (0.758 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (99.3 mg, yield 83%) as a white solid.

¹H NMR (300 MHz, DMSO-d₆) δ 1.24 (3H, q, J=7.5 Hz), 2.37-2.47 (1H, m), 2.58-2.77 (3H, m), 3.55-3.69 (1H, m), 4.12 (1H, t, J=7.2 Hz), 4.20-4.29 (1H, m), 4.63 (1H, t, J=8.7 Hz), 4.73-4.86 (1H, m), 5.32-5.44 (1H, m), 6.14-6.26 (3H, m), 6.79-6.89 (1H, m), 6.96 (1H, dd, J=7.9, 2.6 Hz), 7.01-7.17 (2H, m), 7.43 (1H, d, J=7.9 Hz), 7.53 (1H, d, J=7.5 Hz), 7.65 (1H, dd, J=8.7, 4.9 Hz), 12.31 (1H, br s).

MS m/z 474 (M+H)⁺.

### Example 23

### sodium [(3S)-6-{[(3S)-7-(2-ethyl-6-fluoro-1H-benzimidazol-1-yl)-2,3-dihydro-1-benzofuran-3-yl]amino}-2,3-dihydro-1-benzofuran-3-yl]acetate

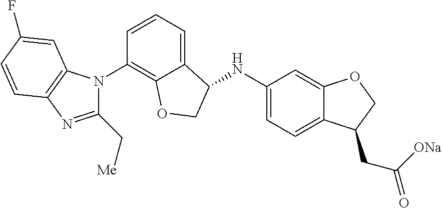

To a mixed solution of [(3S)-6-{[(3S)-7-(2-ethyl-6-fluoro-1H-benzimidazol-1-yl)-2,3-dihydro-1-benzofuran-3-yl]amino}-2,3-dihydro-1-benzofuran-3-yl]acetic acid (93.8 mg, 0.198 mmol) in tetrahydrofuran (2.0 mL) and acetonitrile (2.0 mL) was added 1 M aqueous sodium hydroxide solution (0.198 mL), and the mixture was stirred at room temperature. After dissolution of insoluble material, acetonitrile was added and the mixture was concentrated under reduced pressure to give a solid. This was triturated with acetonitrile to give the title compound (87.3 mg, yield 89%) as a white solid.

¹H NMR (300 MHz, DMSO-d₆) δ 1.24 (3H, q, J=7.7 Hz), 1.96 (1H, dd, J=15.0, 10.0 Hz), 2.30 (1H, dd, J=15.0, 4.4 Hz), 2.62-2.78 (2H, m), 3.46-3.62 (1H, m), 4.07 (1H, t, J=8.0 Hz), 4.19-4.30 (1H, m), 4.60 (1H, t, J=8.9 Hz), 4.79 (1H, q, J=9.3 Hz), 5.30-5.43 (1H, m), 6.02-6.21 (3H, m), 6.78-6.97 (2H, m), 7.00-7.17 (2H, m), 7.42 (1H, d, J=8.0 Hz), 7.53 (1H, d, J=7.6 Hz), 7.65 (1H, dd, J=8.5, 4.7 Hz).

MS m/z 474 (M+H)⁺ (as free form).

### Example 24

### [(3S)-6-{[(3S)-7-(2-ethoxy-6-fluoro-1H-benzimidazol-1-yl)-2,3-dihydro-1-benzofuran-3-yl]amino}-2,3-dihydro-1-benzofuran-3-yl]acetic acid

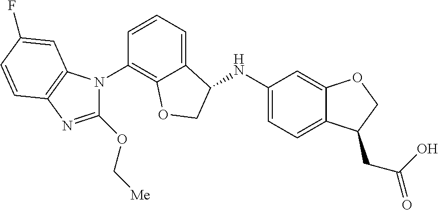

To a mixed solution of methyl [(3S)-6-{[(3S)-7-(2-ethoxy-6-fluoro-1H-benzimidazol-1-yl)-2,3-dihydro-1-benzofuran-3-yl](trifluoroacetyl)amino}-2,3-dihydro-1-benzofuran-3-yl]acetate (2.49 g, 4.15 mmol) in tetrahydrofuran (26 mL) and methanol (13 mL) was added 1 M aqueous sodium hydroxide solution (12.4 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (2.00 g, yield 99%) as a pale-pink solid.

¹H NMR (300 MHz, DMSO-d₆) δ 1.34 (3H, t, J=7.0 Hz), 2.37-2.47 (1H, m), 2.61-2.72 (1H, m), 3.54-3.69 (1H, m), 4.12 (1H, dd, J=9.0, 6.8 Hz), 4.25 (1H, br s), 4.53 (2H, q, J=6.8 Hz), 4.63 (1H, t, J=9.0 Hz), 4.70-4.97 (1H, m), 5.31-5.43 (1H, m), 6.13-6.28 (3H, m), 6.82 (1H, br s), 6.92-7.05 (2H, m), 7.08 (1H, t, J=7.7 Hz), 7.39 (1H, d, J=7.9 Hz), 7.43-7.52 (2H, m), 12.32 (1H, br s).

MS m/z 490 (M+H)⁺.

### Example 25

### sodium [(3S)-6-{[(3S)-7-(2-ethoxy-6-fluoro-1H-benzimidazol-1-yl)-2,3-dihydro-1-benzofuran-3-yl]amino}-2,3-dihydro-1-benzofuran-3-yl]acetate

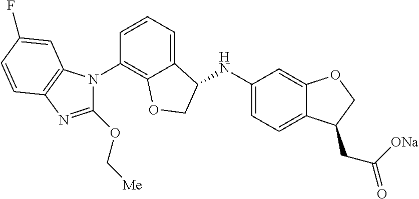

To a mixed solution of [(3S)-6-{[(3S)-7-(2-ethoxy-6-fluoro-1H-benzimidazol-1-yl)-2,3-dihydro-1-benzofuran-3-yl]amino}-2,3-dihydro-1-benzofuran-3-yl]acetic acid (1.70 g, 3.47 mmol) in tetrahydrofuran (18 mL) and acetonitrile (18 mL) was added 1 M aqueous sodium hydroxide solution (3.47 mL), and the mixture was stirred at room temperature. After dissolution of insoluble material, acetonitrile was added and the mixture was concentrated under reduced pressure to give a solid. This was recrystallized from methanol-acetonitrile to give the title compound (1.30 g, yield 73%) as a white solid.

¹H NMR (300 MHz, DMSO-d₆) δ 1.34 (3H, t, J=7.0 Hz), 1.96 (1H, dd, J=15.1, 9.8 Hz), 2.30 (1H, dd, J=14.7, 4.9 Hz), 3.44-3.63 (1H, m), 4.07 (1H, t, J=8.7 Hz), 4.16-4.32 (1H, m), 4.47-4.66 (3H, m), 4.70-4.96 (1H, m), 5.30-5.41 (1H, m), 6.02-6.22 (3H, m), 6.74-7.13 (4H, m), 7.38 (1H, d, J=7.9 Hz), 7.43-7.54 (2H, m).

elemental analysis value for C₂₇H₂₃N₃O₅FNa

Calculated: C, 63.40; H, 4.53; N, 8.22.

Found: C, 63.28; H, 4.30; N, 8.19.

MS m/z 490 (M+H)⁺ (as free form).

melting point not less than 300° C.

### Example 26

### [(3S)-6-{[(3S)-7-(2-ethyl-5,6-difluoro-1H-benzimidazol-1-yl)-2,3-dihydro-1-benzofuran-3-yl]amino}-2,3-dihydro-1-benzofuran-3-yl]acetic acid

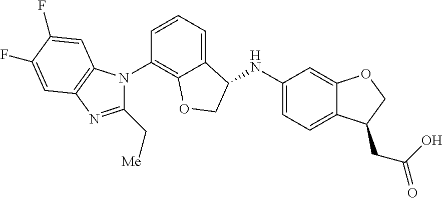

To a mixed solution of methyl [(3S)-6-{[(3S)-7-(2-ethyl-5,6-difluoro-1H-benzimidazol-1-yl)-2,3-dihydro-1-benzofuran-3-yl](trifluoroacetyl)amino}-2,3-dihydro-1-benzofuran-3-yl]acetate (130 mg, 0.215 mmol) in tetrahydrofuran (1.4 mL) and methanol (0.7 mL) was added 1 M aqueous sodium hydroxide solution (0.646 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (89.1 mg, yield 84%) as a white solid.

¹H NMR (300 MHz, DMSO-d₆) δ 1.19-1.29 (3H, m), 2.36-2.46 (1H, m), 2.58-2.79 (3H, m), 3.53-3.69 (1H, m), 4.08-4.17 (1H, m), 4.20-4.30 (1H, m), 4.63 (1H, td, J=9.1, 1.9 Hz), 4.72-4.87 (1H, m), 5.32-5.44 (1H, m), 6.11-6.26 (3H, m), 6.96 (1H, dd, J=8.0, 3.4 Hz), 7.05-7.17 (2H, m), 7.43 (1H, dd, J=7.4, 2.5 Hz), 7.54 (1H, d, J=7.2 Hz), 7.67-7.78 (1H, m).

MS m/z 492 (M+H)⁺.

### Example 27

### sodium [(3S)-6-{[(3S)-7-(2-ethyl-5,6-difluoro-1H-benzimidazol-1-yl)-2,3-dihydro-1-benzofuran-3-yl]amino}-2,3-dihydro-1-benzofuran-3-yl]acetate

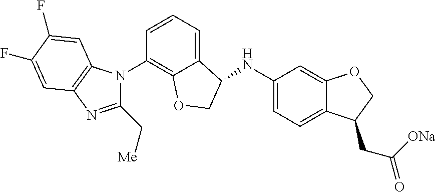

To a suspension of [(3S)-6-{[(3S)-7-(2-ethyl-5,6-difluoro-1H-benzimidazol-1-yl)-2,3-dihydro-1-benzofuran-3-yl]amino}-2,3-dihydro-1-benzofuran-3-yl]acetic acid (89.1 mg, 0.181 mmol) in water (1.0 mL) was added 1 M aqueous sodium hydroxide solution (0.181 mL), and the mixture was stirred at room temperature. After dissolution of insoluble material, acetonitrile was added and the mixture was concentrated under reduced pressure to give the title compound (82.2 mg, yield 88%) as a white solid.

¹H NMR (300 MHz, DMSO-d₆) δ 1.12-1.35 (3H, m), 1.90-2.06 (1H, m), 2.25-2.41 (1H, m), 2.60-2.80 (2H, m), 3.49-3.65 (1H, m), 3.98-4.17 (1H, m), 4.18-4.36 (1H, m), 4.51-4.69 (1H, m), 4.71-4.90 (1H, m), 5.29-5.45 (1H, m), 5.99-6.28 (3H, m), 6.88-7.01 (1H, m), 7.04-7.21 (2H, m), 7.42 (1H, d, J=6.4 Hz), 7.54 (1H, d, J=6.8 Hz), 7.67-7.82 (1H, m).

MS m/z 492 (M+H)⁺ (as free form).

### Example 28

### [(3S)-6-{[(3S)-7-(2-ethoxy-5,6-difluoro-1H-benzimidazol-1-yl)-2,3-dihydro-1-benzofuran-3-yl]amino}-2,3-dihydro-1-benzofuran-3-yl]acetic acid

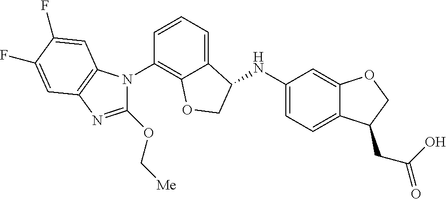

To a mixed solution of methyl [(3S)-6-{[(3S)-7-(2-ethoxy-5,6-difluoro-1H-benzimidazol-1-yl)-2,3-dihydro-1-benzofuran-3-yl](trifluoroacetyl)amino}-2,3-dihydro-1-benzofuran-3-yl]acetate (98.6 mg, 0.160 mmol) in tetrahydrofuran (1.0 mL) and methanol (0.5 mL) was added 1 M aqueous sodium hydroxide solution (0.479 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (100 mg, yield 100%) as a pale-purple solid.

¹H NMR (300 MHz, CDCl₃) δ 1.39-1.46 (4H, m), 2.62 (1H, dd, J=16.7, 9.1 Hz), 2.75-2.86 (1H, m), 3.73-3.87 (1H, m), 4.29 (1H, dd, J=9.3, 5.9 Hz), 4.44 (1H, dd, J=9.7, 4.4 Hz), 4.60 (2H, q, J=7.2 Hz), 4.71-4.84 (2H, m), 5.31 (1H, dd, J=7.2, 4.2 Hz), 6.14-6.21 (2H, m), 6.80 (1H, dd, J=9.8, 7.2 Hz), 6.99-7.12 (2H, m), 7.25-7.32 (2H, m), 7.36 (1H, dd, J=10.6, 7.2 Hz), 7.47 (1H, d, J=7.6 Hz).

MS m/z 508 (M+H)⁺.

### Example 29

### sodium [(3S)-6-{[(3S)-7-(2-ethoxy-5,6-difluoro-1H-benzimidazol-1-yl)-2,3-dihydro-1-benzofuran-3-yl]amino}-2,3-dihydro-1-benzofuran-3-yl]acetate

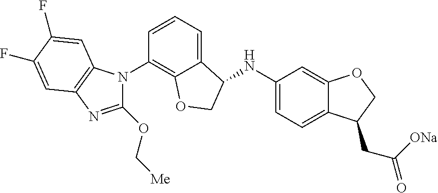

To a suspension of [(3S)-6-{[(3S)-7-(2-ethoxy-5,6-difluoro-1H-benzimidazol-1-yl)-2,3-dihydro-1-benzofuran-3-yl]amino}-2,3-dihydro-1-benzofuran-3-yl]acetic acid (0.160 mmol) in water (1.0 mL) was added 1 M aqueous sodium hydroxide solution (0.160 mL), and the mixture was stirred at room temperature. After dissolution of insoluble material, acetonitrile was added and the mixture was concentrated under reduced pressure to give the title compound (80.9 mg, yield 95%) as a pale-pink solid.

¹H NMR (300 MHz, DMSO-d₆) δ 1.34 (3H, t, J=7.0 Hz), 1.98 (1H, dd, J=15.1, 9.8 Hz), 2.32 (1H, dd, J=15.1, 4.9 Hz), 3.46-3.62 (1H, m), 4.03-4.13 (1H, m), 4.17-4.36 (1H, m), 4.48-4.66 (3H, m), 4.68-4.94 (1H, m), 5.29-5.41 (1H, m), 5.98-6.23 (3H, m), 6.93 (1H, d, J=7.9 Hz), 7.01-7.15 (2H, m), 7.39 (1H, d, J=7.9 Hz), 7.48 (1H, d, J=7.5 Hz), 7.57 (1H, dd, J=11.1, 7.3 Hz).

MS m/z 508 (M+H)⁺ (as free form).

### Example 30

### [(3S)-6-{[(3S)-7-(2-ethyl-6,7-difluoro-1H-benzimidazol-1-yl)-2,3-dihydro-1-benzofuran-3-yl]amino}-2,3-dihydro-1-benzofuran-3-yl]acetic acid

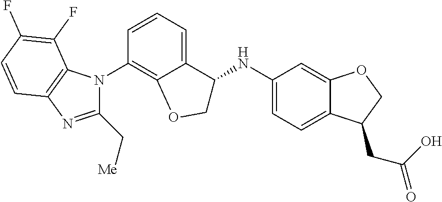

To a mixed solution of methyl [(3S)-6-{[(3S)-7-(2-ethyl-6,7-difluoro-1H-benzimidazol-1-yl)-2,3-dihydro-1-benzofuran-3-yl](trifluoroacetyl)amino}-2,3-dihydro-1-benzofuran-3-yl]acetate (3.72 g, 6.19 mmol) in tetrahydrofuran (40 mL) and methanol (20 mL) was added 1 M aqueous sodium hydroxide solution (18.6 mL), and the mixture was stirred at 50° C. for 2 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. This was triturated with hexane-ethyl acetate to give the title compound (2.44 g, yield 80%) as a white solid.

¹H NMR (300 MHz, DMSO-d₆) δ 1.22 (3H, t, J=7.6 Hz), 2.37-2.47 (1H, m), 2.57-2.71 (3H, m), 3.54-3.68 (1H, m), 4.12 (1H, dd, J=9.1, 6.8 Hz), 4.22 (1H, dd, J=9.3, 5.5 Hz), 4.62 (1H, t, J=9.1 Hz), 4.86 (1H, t, J=8.5 Hz), 5.33-5.45 (1H, m), 6.16-6.30 (3H, m), 6.95 (1H, d, J=7.6 Hz), 7.10 (1H, t, J=7.8 Hz), 7.19-7.33 (1H, m), 7.42-7.57 (3H, m), 12.30 (1H, br s).

MS m/z 492 (M+H)⁺.

### Example 31

### sodium [(3S)-6-{[(3S)-7-(2-ethyl-6,7-difluoro-1H-benzimidazol-1-yl)-2,3-dihydro-1-benzofuran-3-yl]amino}-2,3-dihydro-1-benzofuran-3-yl]acetate

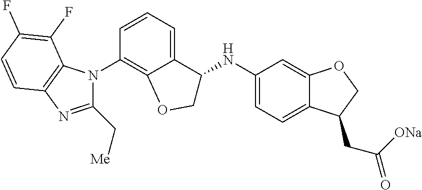

To a mixed solution of [(3S)-6-{[(3S)-7-(2-ethyl-6,7-difluoro-1H-benzimidazol-1-yl)-2,3-dihydro-1-benzofuran-3-yl]amino}-2,3-dihydro-1-benzofuran-3-yl]acetic acid (2.00 g, 4.07 mmol) in tetrahydrofuran (30 mL) and acetonitrile (30 mL) was added 1 M aqueous sodium hydroxide solution (4.07 mL), and the mixture was stirred at room temperature. After dissolution of insoluble material, acetonitrile was added and the mixture was concentrated under reduced pressure to give a solid. This was triturated with acetonitrile and recrystallized from methanol-acetonitrile to give the title compound (1.10 g, yield 53%) as a white solid.

¹H NMR (300 MHz, DMSO-d₆) δ 1.22 (3H, t, J=7.5 Hz), 1.93 (1H, dd, J=15.1, 10.2 Hz), 2.27 (1H, dd, J=15.1, 4.9 Hz), 2.63 (2H, q, J=7.4 Hz), 3.44-3.58 (1H, m), 4.05 (1H, dd, J=8.7, 7.2 Hz), 4.22 (1H, dd, J=9.0, 5.7 Hz), 4.59 (1H, t, J=8.9 Hz), 4.85 (1H, t, J=8.7 Hz), 5.32-5.45 (1H, m), 6.07-6.21 (3H, m), 6.91 (1H, d, J=7.5 Hz), 7.09 (1H, t, J=7.5 Hz), 7.20-7.32 (1H, m), 7.45-7.56 (3H, m).

MS m/z 492 (M+H)⁺ (as free form).

elemental analysis value for C₂₇H₂₂N₃O₄F₂Na

Calculated: C, 63.16; H, 4.32; N, 8.18.

Found: C, 63.10; H, 4.33; N, 8.06.

melting point not less than 300° C.

### Example 32

### [(3S)-6-{[(3S)-7-(2-ethoxy-6,7-difluoro-1H-benzimidazol-1-yl)-2,3-dihydro-1-benzofuran-3-yl]amino}-2,3-dihydro-1-benzofuran-3-yl]acetic acid

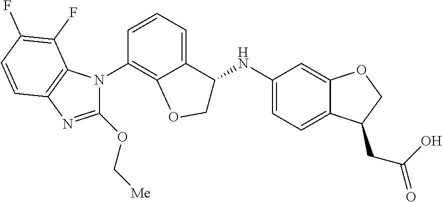

To a mixed solution of methyl [(3S)-6-{[(3S)-7-(2-ethoxy-6,7-difluoro-1H-benzimidazol-1-yl)-2,3-dihydro-1-benzofuran-3-yl](trifluoroacetyl)amino}-2,3-dihydro-1-benzofuran-3-yl]acetate (1.87 g, 3.02 mmol) in tetrahydrofuran (20 mL) and methanol (10 mL) was added 1 M aqueous sodium hydroxide solution (9.07 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. The solid was triturated with hexane-ethyl acetate to give the title compound (1.35 g, yield 88%) as a white solid.

¹H NMR (300 MHz, DMSO-d₆) δ 1.33 (3H, td, J=7.1, 3.6 Hz), 2.42 (1H, dd, J=16.3, 8.7 Hz), 2.64 (1H, dd, J=16.5, 5.5 Hz), 3.52-3.68 (1H, m), 4.11 (1H, dd, J=8.7, 6.8 Hz), 4.16-4.29 (1H, m), 4.45-4.67 (3H, m), 4.74-4.90 (1H, m), 5.31-5.43 (1H, m), 6.15-6.27 (3H, m), 6.95 (1H, d, J=8.0 Hz), 7.06 (1H, t, J=7.8 Hz), 7.11-7.24 (1H, m), 7.26-7.33 (1H, m), 7.47 (2H, d, J=8.3 Hz), 12.11-12.49 (1H, m).

MS m/z 508 (M+H)⁺.

### Example 33

### sodium [(3S)-6-{[(3S)-7-(2-ethoxy-6,7-difluoro-1H-benzimidazol-1-yl)-2,3-dihydro-1-benzofuran-3-yl]amino}-2,3-dihydro-1-benzofuran-3-yl]acetate

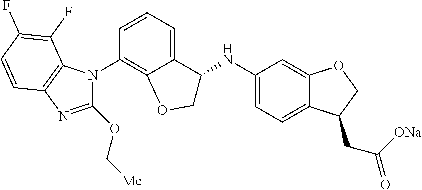

To a mixed solution of [(3S)-6-{[(3S)-7-(2-ethoxy-6,7-difluoro-1H-benzimidazol-1-yl)-2,3-dihydro-1-benzofuran-3-yl]amino}-2,3-dihydro-1-benzofuran-3-yl]acetic acid (1.10 g, 2.17 mmol) in tetrahydrofuran (15 mL) and acetonitrile (15 mL) was added 1 M aqueous sodium hydroxide solution (2.17 mL), and the mixture was stirred at room temperature. After dissolution of insoluble material, acetonitrile was added and the mixture was concentrated under reduced pressure to give a solid. The solid was triturated with acetonitrile and recrystallized from methanol-acetonitrile to give the title compound (803 mg, yield 70%) as a white solid.

¹H NMR (300 MHz, DMSO-d₆) δ 1.33 (3H, td, J=7.2, 3.4 Hz), 1.96 (1H, dd, J=15.1, 9.8 Hz), 2.30 (1H, dd, J=15.1, 4.9 Hz), 3.45-3.60 (1H, m), 4.06 (1H, t, J=8.1 Hz), 4.17-4.29 (1H, m), 4.46-4.65 (3H, m), 4.81 (1H, dt, J=13.3, 8.6 Hz), 5.30-5.42 (1H, m), 6.06-6.21 (3H, m), 6.92 (1H, d, J=7.9 Hz), 7.05 (1H, t, J=7.5 Hz), 7.11-7.25 (1H, m), 7.26-7.34 (1H, m), 7.43-7.52 (2H, m).

MS m/z 508 (M+H)⁺ (as free form).

elemental analysis value for C₂₇H₂₂N₃O₅F₂Na

Calculated: C, 61.25; H, 4.19; N, 7.94.

Found: C, 61.01; H, 4.25; N, 7.98.

melting point not less than 300° C.

### Example 34

### [(3S)-6-{[(3S)-7-(2-ethyl-4,6-difluoro-1H-benzimidazol-1-yl)-2,3-dihydro-1-benzofuran-3-yl]amino}-2,3-dihydro-1-benzofuran-3-yl]acetic acid

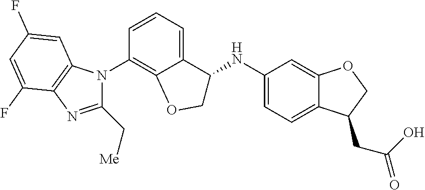

To a mixed solution of methyl [(3S)-6-{[(3S)-7-(2-ethyl-4,6-difluoro-1H-benzimidazol-1-yl)-2,3-dihydro-1-benzofuran-3-yl](trifluoroacetyl)amino}-2,3-dihydro-1-benzofuran-3-yl]acetate (106 mg, 0.177 mmol) in tetrahydrofuran (1.0 mL) and methanol (0.5 mL) was added 1 M aqueous sodium hydroxide solution (0.531 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (89.1 mg, yield 100%) as a colorless oil.

¹H NMR (300 MHz, CDCl₃) δ 1.31-1.41 (3H, m), 2.55-2.89 (4H, m), 3.74-3.89 (1H, m), 4.29 (1H, dd, J=9.3, 5.9 Hz), 4.37-4.49 (1H, m), 4.70-4.85 (2H, m), 5.34 (1H, td, J=7.5, 4.7 Hz), 6.13-6.21 (2H, m), 6.51-6.63 (1H, m), 6.76 (1H, td, J=10.0, 2.3 Hz), 7.03 (1H, d, J=7.6 Hz), 7.06-7.17 (1H, m), 7.21-7.28 (2H, m), 7.54 (1H, d, J=7.2 Hz).

MS m/z 492 (M+H)⁺.

### Example 35

### sodium [(3S)-6-{[(3S)-7-(2-ethyl-4,6-difluoro-1H-benzimidazol-1-yl)-2,3-dihydro-1-benzofuran-3-yl]amino}-2,3-dihydro-1-benzofuran-3-yl]acetate

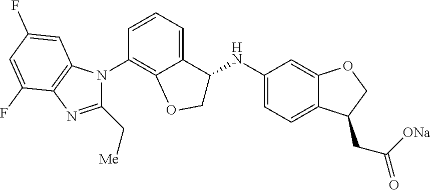

To a suspension of [(3S)-6-{[(3S)-7-(2-ethyl-4,6-difluoro-1H-benzimidazol-1-yl)-2,3-dihydro-1-benzofuran-3-yl]amino}-2,3-dihydro-1-benzofuran-3-yl]acetic acid (0.177 mmol) in water (1.0 mL) was added 1 M aqueous sodium hydroxide solution (0.177 mL), and the mixture was stirred at room temperature. After dissolution of insoluble material, acetonitrile was added and the mixture was concentrated under reduced pressure to give the title compound (76.1 mg, yield 84%) as a white solid.

¹H NMR (300 MHz, DMSO-d₆) δ 1.24 (3H, q, J=7.7 Hz), 1.99 (1H, dd, J=14.9, 10.0 Hz), 2.33 (1H, dd, J=15.1, 4.5 Hz), 2.64-2.80 (2H, m), 3.48-3.63 (1H, m), 4.08 (1H, t, J=7.9 Hz), 4.20-4.31 (1H, m), 4.62 (1H, t, J=9.0 Hz), 4.73-4.87 (1H, m), 5.31-5.44 (1H, m), 6.01-6.22 (3H, m), 6.70-6.82 (1H, m), 6.94 (1H, d, J=7.5 Hz), 7.03-7.18 (2H, m), 7.44 (1H, d, J=7.5 Hz), 7.55 (1H, d, J=7.2 Hz).

MS m/z 492 (M+H)⁺ (as free form).

### Example 36

### [(3S)-6-{[(3S)-7-(2-ethoxy-4,6-difluoro-1H-benzimidazol-1-yl)-2,3-dihydro-1-benzofuran-3-yl]amino}-2,3-dihydro-1-benzofuran-3-yl]acetic acid

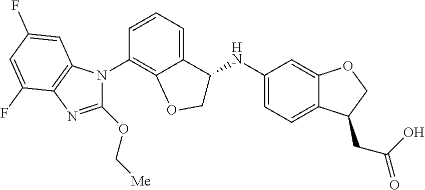

To a mixed solution of methyl [(3S)-6-{[(3S)-7-(2-ethoxy-4,6-difluoro-1H-benzimidazol-1-yl)-2,3-dihydro-1-benzofuran-3-yl](trifluoroacetyl)amino}-2,3-dihydro-1-benzofuran-3-yl]acetate (2.04 g, 3.30 mmol) in tetrahydrofuran (21 mL) and methanol (10 mL) was added 1 M aqueous sodium hydroxide solution (9.91 mL), and the mixture was stirred at 50° C. for 2 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give a solid. The solid was recrystallized from hexane-ethyl acetate to give the title compound (1.48 g, yield 88%) as a pale-pink solid.

¹H NMR (300 MHz, DMSO-d₆) δ 1.35 (3H, t, J=7.2 Hz), 2.38-2.47 (1H, m), 2.65 (1H, dd, J=16.3, 5.3 Hz), 3.55-3.69 (1H, m), 4.12 (1H, dd, J=8.7, 6.8 Hz), 4.17-4.32 (1H, m), 4.49-4.68 (3H, m), 4.70-4.98 (1H, m), 5.30-5.43 (1H, m), 6.14-6.30 (3H, m), 6.74 (1H, br s), 6.96 (1H, d, J=7.6 Hz), 7.00-7.14 (2H, m), 7.41 (1H, d, J=7.6 Hz), 7.49 (1H, d, J=7.2 Hz), 12.32 (1H, br s).

MS m/z 508 (M+H)⁺.

### Example 37

### sodium [(3S)-6-{[(3S)-7-(2-ethoxy-4,6-difluoro-1H-benzimidazol-1-yl)-2,3-dihydro-1-benzofuran-3-yl]amino}-2,3-dihydro-1-benzofuran-3-yl]acetate

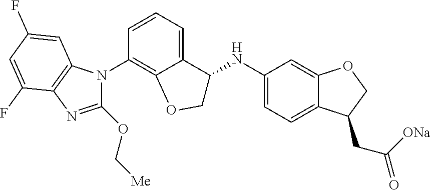

To a mixed solution of [(3S)-6-{[(3S)-7-(2-ethoxy-4,6-difluoro-1H-benzimidazol-1-yl)-2,3-dihydro-1-benzofuran-3-yl]amino}-2,3-dihydro-1-benzofuran-3-yl]acetic acid (1.48 g, 2.92 mmol) in tetrahydrofuran (20 mL) and acetonitrile (20 mL) was added 1 M aqueous sodium hydroxide solution (2.92 mL), and the mixture was stirred at room temperature. After dissolution of insoluble material, acetonitrile was added and the mixture was concentrated under reduced pressure to give a solid. The solid was triturated with acetonitrile and recrystallized from methanol-acetonitrile to give the title compound (1.07 g, yield 69%) as a white solid.

¹H NMR (300 MHz, DMSO-d₆) δ 1.35 (3H, t, J=7.2 Hz), 1.97 (1H, dd, J=15.0, 10.0 Hz), 2.31 (1H, dd, J=15.1, 4.9 Hz), 3.45-3.60 (1H, m), 4.00-4.12 (1H, m), 4.18-4.33 (1H, m), 4.50-4.65 (3H, m), 4.70-4.94 (1H, m), 5.30-5.41 (1H, m), 6.01-6.22 (3H, m), 6.68-6.80 (1H, m), 6.93 (1H, d, J=7.6 Hz), 7.00-7.13 (2H, m), 7.40 (1H, d, J=7.6 Hz), 7.49 (1H, d, J=7.2 Hz).

MS m/z 508 (M+H)⁺ (as free form).

### Example 38

### [(3S)-6-{[(3S)-7-(2-ethyl-6-methoxy-1H-benzimidazol-1-yl)-2,3-dihydro-1-benzofuran-3-yl]amino}-2,3-dihydro-1-benzofuran-3-yl]acetic acid

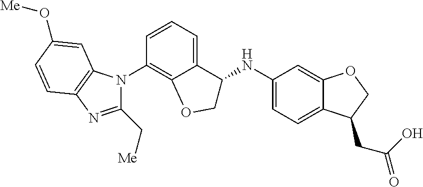

To a mixed solution of methyl [(3S)-6-{[(3S)-7-(2-ethyl-6-methoxy-1H-benzimidazol-1-yl)-2,3-dihydro-1-benzofuran-3-yl](trifluoroacetyl)amino}-2,3-dihydro-1-benzofuran-3-yl]acetate(101 mg, 0.169 mmol) in tetrahydrofuran (1.0 mL) and methanol (0.5 mL) was added 1 M aqueous sodium hydroxide solution (0.507 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (86.0 mg, yield 100%) as a colorless oil.

¹H NMR (300 MHz, CDCl₃) δ 1.29-1.39 (3H, m), 2.55-2.89 (4H, m), 3.72-3.89 (4H, m), 4.30 (1H, dd, J=9.2, 6.2 Hz), 4.36-4.47 (1H, m), 4.71-4.84 (2H, m), 5.27-5.39 (1H, m), 6.13-6.20 (2H, m), 6.53 (1H, dd, J=15.6, 2.4 Hz), 6.85-6.93 (1H, m), 7.03 (1H, d, J=8.3 Hz), 7.07-7.16 (1H, m), 7.26-7.29 (1H, m), 7.52 (1H, d, J=7.5 Hz), 7.67 (1H, d, J=9.0 Hz).

MS m/z 486 (M+H)⁺.

### Example 39

### sodium [(3S)-6-{[(3S)-7-(2-ethyl-6-methoxy-1H-benzimidazol-1-yl)-2,3-dihydro-1-benzofuran-3-yl]amino}-2,3-dihydro-1-benzofuran-3-yl]acetate

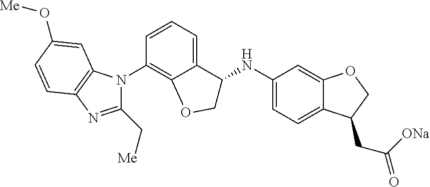

To a suspension of [(3S)-6-{[(3S)-7-(2-ethyl-6-methoxy-1H-benzimidazol-1-yl)-2,3-dihydro-1-benzofuran-3-yl]amino}-2,3-dihydro-1-benzofuran-3-yl]acetic acid (0.169 mmol) in water (1.0 mL) was added 1 M aqueous sodium hydroxide solution (0.169 mL), and the mixture was stirred at room temperature. After dissolution of insoluble material, acetonitrile was added and the mixture was concentrated under reduced pressure to give the title compound (63.8 mg, yield 74%) as a white solid.

¹H NMR (300 MHz, DMSO-d₆) δ 1.16-1.28 (3H, m), 2.00 (1H, dd, J=15.1, 9.8 Hz), 2.33 (1H, dd, J=15.1, 4.9 Hz), 2.58-2.74 (2H, m), 3.46-3.62 (1H, m), 3.70 (3H, d, J=2.6 Hz), 4.02-4.12 (1H, m), 4.18-4.30 (1H, m), 4.61 (1H, t, J=8.9 Hz), 4.80 (1H, t, J=8.7 Hz), 5.31-5.44 (1H, m), 6.07-6.22 (3H, m), 6.50 (1H, dd, J=17.7, 2.3 Hz), 6.78-6.87 (1H, m), 6.93 (1H, d, J=7.9 Hz), 7.13 (1H, t, J=7.5 Hz), 7.32-7.44 (1H, m), 7.52 (2H, d, J=8.7 Hz).

MS m/z 486 (M+H)⁺ (as free form).

### Example 40

### [(3S)-6-({(3S)-7-[6-fluoro-2-(propan-2-yl)-1H-benzimidazol-1-yl]-2,3-dihydro-1-benzofuran-3-yl}amino)-2,3-dihydro-1-benzofuran-3-yl]acetic acid

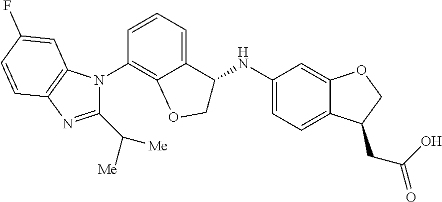

To a mixed solution of methyl {(3S)-6-[{(3S)-7-[6-fluoro-2-(propan-2-yl)-1H-benzimidazol-1-yl]-2,3-dihydro-1-benzofuran-3-yl}(trifluoroacetyl)amino]-2,3-dihydro-1-benzofuran-3-yl}acetate (134 mg, 0.223 mmol) in tetrahydrofuran (1.4 mL) and methanol (0.7 mL) was added 1 M aqueous sodium hydroxide solution (0.670 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (116 mg, yield 100%) as a colorless oil.

¹H NMR (300 MHz, CDCl₃) δ 1.21-1.41 (6H, m), 2.62 (1H, dd, J=16.7, 9.5 Hz), 2.81 (1H, dd, J=17.0, 5.3 Hz), 2.91-3.08 (1H, m), 3.74-3.89 (1H, m), 4.30 (1H, dd, J=9.5, 6.1 Hz), 4.36-4.47 (1H, m), 4.72-4.82 (2H, m), 5.29-5.39 (1H, m), 6.12-6.22 (2H, m), 6.65-6.78 (1H, m), 6.94-7.07 (2H, m), 7.07-7.16 (1H, m), 7.22-7.26 (1H, m), 7.54 (1H, d, J=7.2 Hz), 7.73 (1H, dd, J=8.9, 4.7 Hz).

MS m/z 488 (M+H)⁺.

### Example 41

### sodium [(3S)-6-({(3S)-7-[6-fluoro-2-(propan-2-yl)-1H-benzimidazol-1-yl]-2,3-dihydro-1-benzofuran-3-yl}amino)-2,3-dihydro-1-benzofuran-3-yl]acetate

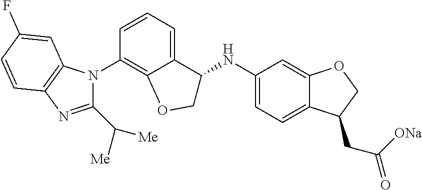

To a suspension of [(3S)-6-({(3S)-7-[6-fluoro-2-(propan-2-yl)-1H-benzimidazol-1-yl]-2,3-dihydro-1-benzofuran-3-yl}amino)-2,3-dihydro-1-benzofuran-3-yl]acetic acid (0.223 mmol) in water (1.0 mL) was added 1 M aqueous sodium hydroxide solution (0.238 mL), and the mixture was stirred at room temperature. After dissolution of insoluble material, acetonitrile was added and the mixture was concentrated under reduced pressure to give the title compound (93.3 mg, yield 82%) as a white solid.

¹H NMR (300 MHz, DMSO-d₆) δ 1.14-1.30 (6H, m), 1.96-2.11 (1H, m), 2.36 (1H, dd, J=15.1, 4.5 Hz), 2.88-3.07 (1H, m), 3.33-3.44 (1H, m), 3.97-4.13 (1H, m), 4.16-4.31 (1H, m), 4.62 (1H, t, J=8.9 Hz), 4.70-4.86 (1H, m), 5.31-5.46 (1H, m), 6.04-6.23 (3H, m), 6.72-7.21 (4H, m), 7.42 (1H, d, J=7.5 Hz), 7.54 (1H, d, J=7.5 Hz), 7.66 (1H, dd, J=8.5, 4.7 Hz).

MS m/z 488 (M+H)⁺ (as free form).

### Example 42

### [(3S)-6-({(3S)-7-[6-fluoro-2-(tetrahydro-2H-pyran-4-yl)-1H-benzimidazol-1-yl]-2,3-dihydro-1-benzofuran-3-yl}amino)-2,3-dihydro-1-benzofuran-3-yl]acetic acid

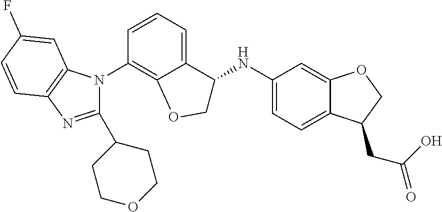

To a mixed solution of methyl {(3S)-6-[{(3S)-7-[6-fluoro-2-(tetrahydro-2H-pyran-4-yl)-1H-benzimidazol-1-yl]-2,3-dihydro-1-benzofuran-3-yl}(trifluoroacetyl)amino]-2,3-dihydro-1-benzofuran-3-yl}acetate (106 mg, 0.165 mmol) in tetrahydrofuran (1.0 mL) and methanol (0.5 mL) was added 1 M aqueous sodium hydroxide solution (0.495 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (89.5 mg, yield 100%) as a pale-yellow oil.

¹H NMR (300 MHz, DMSO-d₆) δ 1.60-1.93 (4H, m), 2.25-2.46 (2H, m), 2.56-2.67 (1H, m), 2.87-3.02 (1H, m), 3.38-3.45 (1H, m), 3.53-3.68 (1H, m), 3.82-3.96 (2H, m), 4.07-4.16 (1H, m), 4.17-4.30 (1H, m), 4.56-4.67 (1H, m), 4.71-4.87 (1H, m), 5.33-5.45 (1H, m), 6.10-6.28 (3H, m), 6.74-6.88 (1H, m), 6.96 (1H, d, J=8.0 Hz), 7.02-7.19 (2H, m), 7.44 (1H, d, J=8.0 Hz), 7.55 (1H, d, J=7.6 Hz), 7.63-7.73 (1H, m).

MS m/z 530 (M+H)⁺.

### Example 43

### sodium [(3S)-6-({(3S)-7-[6-fluoro-2-(tetrahydro-2H-pyran-4-yl)-1H-benzimidazol-1-yl]-2,3-dihydro-1-benzofuran-3-yl}amino)-2,3-dihydro-1-benzofuran-3-yl]acetate

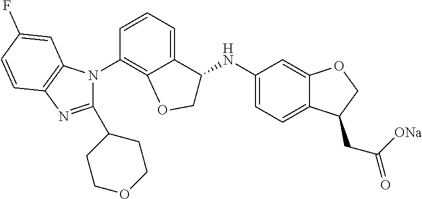

To a suspension of [(3S)-6-({(3S)-7-[6-fluoro-2-(tetrahydro-2H-pyran-4-yl)-1H-benzimidazol-1-yl]-2,3-dihydro-1-benzofuran-3-yl}amino)-2,3-dihydro-1-benzofuran-3-yl]acetic acid (0.165 mmol) in water (1.0 mL) was added 1 M aqueous sodium hydroxide solution (0.165 mL), and the mixture was stirred at room temperature. After dissolution of insoluble material, acetonitrile was added and the mixture was concentrated under reduced pressure to give the title compound (78.0 mg, yield 86%) as a white solid.

¹H NMR (300 MHz, DMSO-d₆) δ 1.70-2.05 (5H, m), 2.27-2.42 (2H, m), 2.82-3.05 (2H, m), 3.77-3.96 (2H, m), 3.98-4.14 (1H, m), 4.15-4.33 (1H, m), 4.50-4.67 (1H, m), 4.68-4.88 (1H, m), 5.28-5.45 (1H, m), 5.97-6.27 (3H, m), 6.71-6.99 (2H, m), 6.99-7.24 (2H, m), 7.36-7.60 (2H, m), 7.61-7.78 (1H, m).

MS m/z 530 (M+H)⁺ (as free form).

### Example 44

### [(3S)-6-({(3S)-7-[6-fluoro-2-(5-methylfuran-2-yl)-1H-benzimidazol-1-yl]-2,3-dihydro-1-benzofuran-3-yl}amino)-2,3-dihydro-1-benzofuran-3-yl]acetic acid

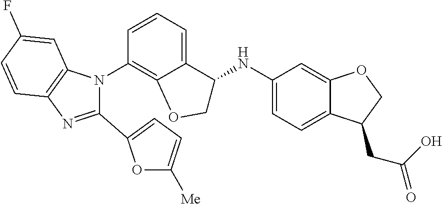

To a mixed solution of methyl {(3S)-6-[{(3S)-7-[6-fluoro-2-(5-methylfuran-2-yl)-1H-benzimidazol-1-yl]-2,3-dihydro-1-benzofuran-3-yl}(trifluoroacetyl)amino]-2,3-dihydro-1-benzofuran-3-yl}acetate (116 mg, 0.182 mmol) in tetrahydrofuran (1.2 mL) and methanol (0.6 mL) was added 1 M aqueous sodium hydroxide solution (0.545 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (99.7 mg, yield 100%) as a pale-yellow oil.

¹H NMR (300 MHz, DMSO-d₆) δ 2.24-2.32 (3H, m), 2.32-2.46 (2H, m), 2.55-2.67 (1H, m), 3.54-3.68 (1H, m), 4.07-4.27 (2H, m), 4.56-4.86 (2H, m), 5.29-5.45 (1H, m), 6.09-6.25 (5H, m), 6.79-6.91 (1H, m), 6.95 (1H, d, J=8.0 Hz), 7.09-7.20 (2H, m), 7.40 (1H, t, J=6.6 Hz), 7.59 (1H, d, J=7.2 Hz), 7.68-7.77 (1H, m).

MS m/z 526 (M+H)⁺.

### Example 45

### sodium [(3S)-6-({(3S)-7-[6-fluoro-2-(5-methylfuran-2-yl)-1H-benzimidazol-1-yl]-2,3-dihydro-1-benzofuran-3-yl}amino)-2,3-dihydro-1-benzofuran-3-yl]acetate

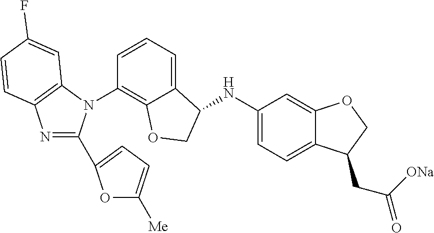

To a suspension of [(3S)-6-({(3S)-7-[6-fluoro-2-(5-methylfuran-2-yl)-1H-benzimidazol-1-yl]-2,3-dihydro-1-benzofuran-3-yl}amino)-2,3-dihydro-1-benzofuran-3-yl]acetic acid (0.182 mmol) in water (1.0 mL) was added 1 M aqueous sodium hydroxide solution (0.182 mL), and the mixture was stirred at room temperature. After dissolution of insoluble material, acetonitrile was added and the mixture was concentrated under reduced pressure to give the title compound (85.5 mg, yield 86%) as a white solid.

¹H NMR (300 MHz, DMSO-d₆) δ 1.99 (1H, dd, J=15.0, 10.0 Hz), 2.24-2.39 (4H, m), 3.48-3.63 (1H, m), 4.07 (1H, t, J=8.0 Hz), 4.11-4.28 (1H, m), 4.54-4.86 (2H, m), 5.28-5.43 (1H, m), 6.06 (1H, t, J=7.6 Hz), 6.10-6.24 (4H, m), 6.80-6.97 (2H, m), 7.07-7.20 (2H, m), 7.40 (1H, t, J=6.8 Hz), 7.59 (1H, d, J=7.2 Hz), 7.67-7.77 (1H, m).

MS m/z 526 (M+H)⁺ (as free form).

### Example 46

### [(3S)-6-({(3S)-7-[6-fluoro-2-(5-methylisoxazol-3-yl)-1H-benzimidazol-1-yl]-2,3-dihydro-1-benzofuran-3-yl}amino)-2,3-dihydro-1-benzofuran-3-yl]acetic acid

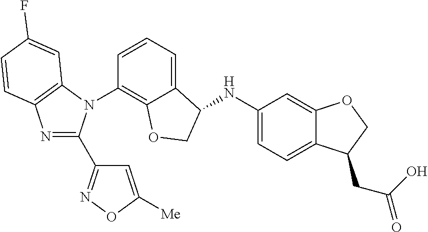

To a mixed solution of methyl {(3S)-6-[{(3S)-7-[6-fluoro-2-(5-methylisoxazol-3-yl)-1H-benzimidazol-1-yl]-2,3-dihydro-1-benzofuran-3-yl}(trifluoroacetyl)amino]-2,3-dihydro-1-benzofuran-3-yl}acetate (104 mg, 0.164 mmol) in tetrahydrofuran (1.0 mL) and methanol (0.5 mL) was added 1 M aqueous sodium hydroxide solution (0.492 mL), and the mixture was stirred at 50° C. for 1 hr. The reaction mixture was neutralized with 1 M hydrochloric acid, diluted with distilled water, and extracted with ethyl acetate. The extract was washed with saturated brine, dried over anhydrous magnesium sulfate, and concentrated under reduced pressure to give the title compound (76.2 mg, yield 88%) as a white solid.

¹H NMR (300 MHz, DMSO-d₆) δ 2.38-2.47 (4H, m), 2.58-2.76 (1H, m), 3.54-3.69 (1H, m), 4.08-4.25 (2H, m), 4.57-4.87 (2H, m), 5.30-5.41 (1H, m), 6.10-6.25 (3H, m), 6.65-6.72 (1H, m), 6.90-7.02 (2H, m), 7.03-7.12 (1H, m), 7.17-7.29 (1H, m), 7.40 (1H, d, J=7.9 Hz), 7.52 (1H, d, J=7.5 Hz), 7.80-7.92 (1H, m).

MS m/z 527 (M+H)⁺.

### Example 47

### sodium [(3S)-6-({(3S)-7-[6-fluoro-2-(5-methylisoxazol-3-yl)-1H-benzimidazol-1-yl]-2,3-dihydro-1-benzofuran-3-yl}amino)-2,3-dihydro-1-benzofuran-3-yl]acetate

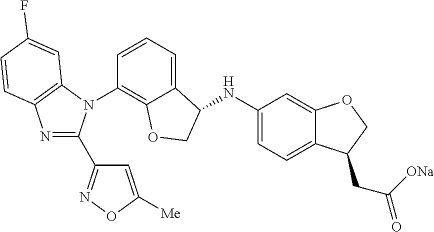

To a suspension of [(3S)-6-({(3S)-7-[6-fluoro-2-(5-methylisoxazol-3-yl)-1H-benzimidazol-1-yl]-2,3-dihydro-1-benzofuran-3-yl}amino)-2,3-dihydro-1-benzofuran-3-yl]acetic acid (76.2 mg, 0.145 mmol) in water (1.0 mL) was added 1 M aqueous sodium hydroxide solution (0.145 mL), and the mixture was stirred at room temperature. After dissolution of insoluble material, acetonitrile was added and the mixture was concentrated under reduced pressure to give the title compound (68.1 mg, yield 86%) as a white solid.

¹H NMR (300 MHz, DMSO-d₆) δ 2.33 (1H, dd, J=14.9, 4.7 Hz), 2.45 (3H, s), 3.47-3.68 (2H, m), 3.97-4.27 (2H, m), 4.54-4.88 (2H, m), 5.26-5.43 (1H, m), 5.97-6.34 (3H, m), 6.62-6.77 (1H, m), 6.87-7.16 (3H, m), 7.17-7.32 (1H, m), 7.39 (1H, d, J=7.9 Hz), 7.53 (1H, d, J=7.2 Hz), 7.83-7.98 (1H, m).

MS m/z 527 (M+H)⁺ (as free form).

### Example 48

### [(3S)-6-({(3S)-7-[4-(cyclopropylmethoxy)-2,6-dimethylphenyl]-2,3-dihydro-1-benzofuran-3-yl}amino)-2,3-dihydro-1-benzofuran-3-yl]acetic acid

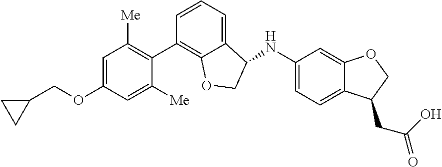

A 0.20 M solution (500 μL, 100 μmol) of methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 in toluene, a 0.40 M solution (500 μL, 200 μmol) of triphenylphosphine in toluene and a 0.30 M solution (500 μL, 150 μmol) of cyclopropanemethanol in toluene were mixed, and to the mixture was added at room temperature diisopropyl azodicarboxylate (38 μL, 200 μmol) and the mixture was stirred for 16 hr. The mixture was neutralized and extracted with ethyl acetate (3.5 mL)-2% aqueous sodium hydrogen carbonate solution (1 mL), and the organic layer was separated by upper layer PhaseSep tube (manufactured by Wako Pure Chemical Industries, Ltd.). The solvent was evaporated under reduced pressure, and the residue was dissolved in DMSO-methanol (1:1, 1 mL), and purified by preparative HPLC. The solvent was evaporated to give purified alkyloxy intermediate. The obtained alkyloxy intermediate was dissolved in tetrahydrofuran-methanol (1:1, 0.5 mL), 2 M aqueous sodium hydroxide solution (0.5 mL) was added, and the mixture was stirred at room temperature for 16 hr. 1 M Aqueous ammonium chloride solution (2.0 mL) was added, and methanol was further added. The residue was completely dissolved, and the solution was purified by preparative HPLC to give the title compound (13.3 mg, yield 27%) as a colorless oil.

¹H NMR (300 MHz, CDCl₃) δ 0.29-0.39 (2H, m), 0.57-0.73 (2H, m), 1.20-1.36 (1H, m), 2.04 (3H, s), 2.07 (3H, s), 2.53-2.62 (1H, m), 2.79 (1H, dd, J=16.7, 5.3 Hz), 3.72-3.89 (3H, m), 4.21-4.40 (2H, m), 4.62-4.80 (2H, m), 5.17-5.29 (1H, m), 6.09-6.24 (2H, m), 6.68 (2H, s), 6.92-7.12 (3H, m), 7.34 (1H, d, J=6.8 Hz).

MS m/z 486 (M+H)⁺.

### Example 49

### [(3S)-6-({(3S)-7-[4-(2-methoxyethoxy)-2,6-dimethylphenyl]-2,3-dihydro-1-benzofuran-3-yl}amino)-2,3-dihydro-1-benzofuran-3-yl]acetic acid

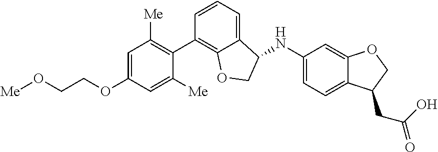

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 2-methoxyethanol.

¹H NMR (300 MHz, CDCl₃) δ 2.04 (3H, s), 2.07 (3H, s), 2.58 (1H, dd, J=16.7, 9.1 Hz), 2.78 (1H, dd, J=16.7, 5.3 Hz), 3.45 (3H, s), 3.71-3.86 (3H, m), 4.09-4.16 (2H, m), 4.21-4.39 (2H, m), 4.61-4.79 (2H, m), 5.17-5.25 (1H, m), 6.08-6.19 (2H, m), 6.70 (2H, s), 6.92-7.07 (3H, m), 7.33 (1H, d, J=6.8 Hz).

MS m/z 490 (M+H)⁺.

### Example 50

### [(3S)-6-{[(3S)-7-{4-[2-(dimethylamino)ethoxy]-2,6-dimethylphenyl}-2,3-dihydro-1-benzofuran-3-yl]amino}-2,3-dihydro-1-benzofuran-3-yl]acetic acid

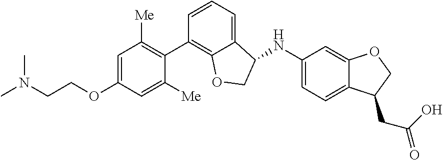

In the same manner as in Example 48, the title compound was obtained as a white solid from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and N,N-dimethylethanolamine.

¹H NMR (300 MHz, CDCl₃) δ 2.03 (3H, s), 2.05 (3H, s), 2.38-2.93 (8H, m), 2.95-3.11 (2H, m), 3.68-3.88 (1H, m), 4.12-4.34 (4H, m), 4.61 (1H, dd, J=9.7, 7.4 Hz), 4.74 (1H, t, J=8.9 Hz), 5.17 (1H, dd, J=7.0, 4.4 Hz), 6.00-6.13 (2H, m), 6.67 (2H, s), 6.91-7.05 (3H, m), 7.30-7.36 (1H, m).

MS m/z 503 (M+H)⁺.

### Example 51

### [(3S)-6-({(3S)-7-[4-(furan-2-ylmethoxy)-2,6-dimethylphenyl]-2,3-dihydro-1-benzofuran-3-yl}amino)-2,3-dihydro-1-benzofuran-3-yl]acetic acid

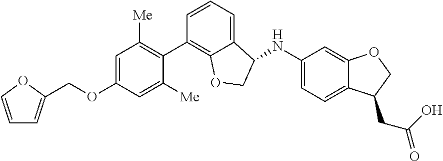

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and furfuryl alcohol.

¹H NMR (300 MHz, CDCl₃) δ 2.05 (3H, s), 2.08 (3H, s), 2.51-2.65 (1H, m), 2.79 (1H, dd, J=16.7, 5.3 Hz), 3.72-3.87 (1H, m), 4.17-4.41 (2H, m), 4.61-4.81 (2H, m), 4.99 (2H, s), 5.22 (1H, dd, J=7.2, 4.2 Hz), 6.11-6.18 (2H, m), 6.34-6.47 (2H, m), 6.76 (2H, s), 6.92-7.08 (3H, m), 7.34 (1H, d, J=6.1 Hz), 7.45 (1H, s).

MS m/z 512 (M+H)⁺.

### Example 52

### [(3S)-6-({(3S)-7-[2,6-dimethyl-4-(pyridin-3-ylmethoxy)phenyl]-2,3-dihydro-1-benzofuran-3-yl}amino)-2,3-dihydro-1-benzofuran-3-yl]acetic acid

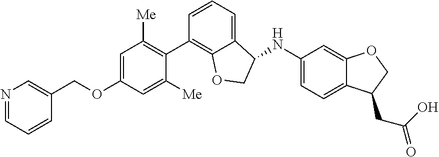

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 3-pyridinemethanol.

¹H NMR (300 MHz, CDCl₃) δ 2.06 (3H, s), 2.09 (3H, s), 2.52-2.64 (1H, m), 2.80 (1H, dd, J=16.7, 5.3 Hz), 3.74-3.89 (1H, m), 4.22-4.39 (2H, m), 4.68 (1H, dd, J=9.7, 7.4 Hz), 4.77 (1H, t, J=8.9 Hz), 5.09 (2H, s), 5.22 (1H, dd, J=7.2, 4.5 Hz), 6.10-6.20 (2H, m), 6.76 (2H, s), 6.93-7.07 (3H, m), 7.31-7.42 (2H, m), 7.84 (1H, d, J=7.6 Hz), 8.60 (1H, dd, J=4.9, 1.5 Hz), 8.70 (1H, d, J=1.9 Hz).

MS m/z 523 (M+H)⁺.

### Example 53

### [(3S)-6-{[(3S)-7-{2,6-dimethyl-4-[2-(2-oxopyrrolidin-1-yl)ethoxy]phenyl}-2,3-dihydro-1-benzofuran-3-yl]amino}-2,3-dihydro-1-benzofuran-3-yl]acetic acid

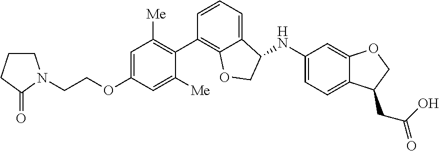

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 1-(2-hydroxyethyl)-2-pyrrolidone.

¹H NMR (300 MHz, CDCl₃) δ 1.98-2.10 (8H, m), 2.34-2.47 (2H, m), 2.60 (1H, dd, J=16.7, 9.1 Hz), 2.79 (1H, dd, J=16.7, 5.3 Hz), 3.61 (2H, t, J=7.2 Hz), 3.69 (2H, t, J=4.9 Hz), 3.73-3.86 (1H, m), 4.11 (2H, t, J=5.1 Hz), 4.22-4.38 (2H, m), 4.67 (1H, dd, J=9.7, 7.4 Hz), 4.74 (1H, t, J=9.1 Hz), 5.22 (1H, dd, J=7.2, 4.2 Hz), 6.09-6.19 (2H, m), 6.59-6.68 (2H, m), 6.92-7.06 (3H, m), 7.34 (1H, d, J=7.2 Hz).

MS m/z 543 (M+H)⁺.

### Example 54

### [(3S)-6-{[(3S)-7-{2,6-dimethyl-4-[3-(pyridin-3-yl)propoxy]phenyl}-2,3-dihydro-1-benzofuran-3-yl]amino}-2,3-dihydro-1-benzofuran-3-yl]acetic acid

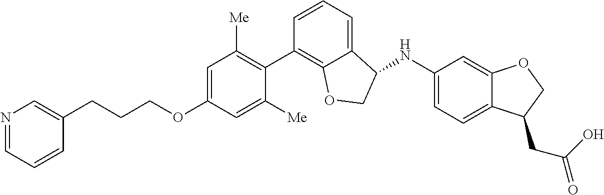

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 3-pyridinepropanol.

¹H NMR (300 MHz, CDCl₃) δ 2.02-2.19 (8H, m), 2.59 (1H, dd, J=16.7, 9.5 Hz), 2.73-2.92 (3H, m), 3.72-3.90 (1H, m), 3.97 (2H, t, J=5.9 Hz), 4.23-4.40 (2H, m), 4.67 (1H, dd, J=9.7, 7.4 Hz), 4.77 (1H, t, J=9.1 Hz), 5.22 (1H, dd, J=7.0, 4.4 Hz), 6.11-6.21 (2H, m), 6.66 (2H, s), 6.90-7.08 (3H, m), 7.28-7.39 (2H, m), 7.62 (1H, d, J=8.0 Hz), 8.44-8.57 (2H, m).

MS m/z 551 (M+H)⁺.

### Example 55

### [(3S)-6-{[(3S)-7-{2,6-dimethyl-4-[3-(2-oxopyrrolidin-1-yl)propoxy]phenyl}-2,3-dihydro-1-benzofuran-3-yl]amino}-2,3-dihydro-1-benzofuran-3-yl]acetic acid

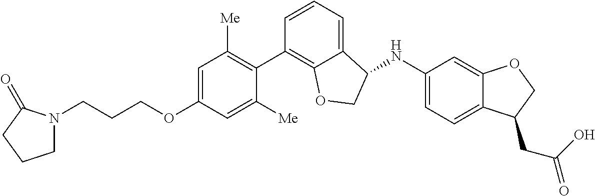

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 1-(3-hydroxypropyl)-2-pyrrolidone.

¹H NMR (300 MHz, CDCl₃) δ 1.96-2.10 (10H, m), 2.37-2.47 (2H, m), 2.56 (1H, dd, J=16.3, 9.1 Hz), 2.77 (1H, dd, J=16.7, 4.9 Hz), 3.46 (4H, q, J=7.4 Hz), 3.71-3.85 (1H, m), 3.98 (2H, t, J=6.2 Hz), 4.20-4.37 (2H, m), 4.66 (1H, dd, J=9.7, 7.4 Hz), 4.74 (1H, t, J=9.1 Hz), 5.21 (1H, dd, J=7.2, 4.2 Hz), 6.08-6.18 (2H, m), 6.66 (2H, s), 6.92-7.05 (3H, m), 7.31-7.37 (1H, m).

MS m/z 557 (M+H)⁺.

### Example 56

### [(3S)-6-{[(3S)-7-{4-[2-(2-fluorophenyl)ethoxy]-2,6-dimethylphenyl}-2,3-dihydro-1-benzofuran-3-yl]amino}-2,3-dihydro-1-benzofuran-3-yl]acetic acid

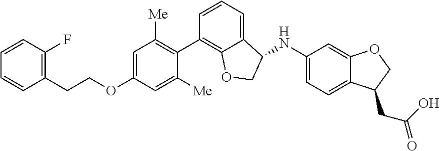

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 2-fluorophenethyl alcohol.

¹H NMR (300 MHz, CDCl₃) δ 2.03 (3H, s), 2.06 (3H, s), 2.56 (1H, dd, J=16.7, 9.5 Hz), 2.76 (1H, dd, J=16.7, 5.3 Hz), 3.13 (2H, t, J=6.8 Hz), 3.69-3.85 (1H, m), 4.18 (2H, t, J=6.8 Hz), 4.22-4.38 (2H, m), 4.65 (1H, dd, J=9.5, 7.6 Hz), 4.72 (1H, t, J=9.1 Hz), 5.20 (1H, dd, J=7.2, 4.2 Hz), 6.09-6.18 (2H, m), 6.67 (2H, s), 6.89-7.13 (5H, m), 7.15-7.24 (1H, m), 7.27-7.37 (2H, m).

MS m/z 554 (M+H)⁺.

### Example 57

### [(3S)-6-{[(3S)-7-{2,6-dimethyl-4-[2-(4-methylpiperazin-1-yl)ethoxy]phenyl}-2,3-dihydro-1-benzofuran-3-yl]amino}-2,3-dihydro-1-benzofuran-3-yl]acetic acid

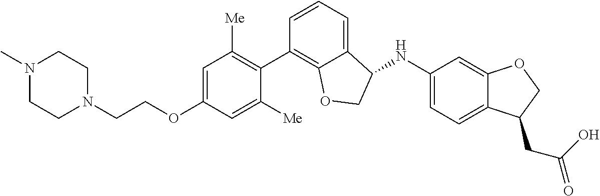

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 1-(2-hydroxyethyl)-4-methylpiperazine.

¹H NMR (300 MHz, CDCl₃) δ 2.03 (3H, s), 2.06 (3H, s), 2.40-2.57 (4H, m), 2.65-2.85 (9H, m), 2.89 (2H, t, J=5.1 Hz), 3.69-3.88 (1H, m), 4.13 (2H, t, J=5.1 Hz), 4.20-4.35 (2H, m), 4.64 (1H, dd, J=9.5, 7.6 Hz), 4.76 (1H, t, J=9.1 Hz), 5.20 (1H, dd, J=7.0, 4.4 Hz), 6.06-6.18 (2H, m), 6.66 (2H, s), 6.90-7.06 (3H, m), 7.33 (1H, d, J=5.3 Hz).

MS m/z 558 (M+H)⁺.

### Example 58

### [(3S)-6-{[(3S)-7-{2,6-dimethyl-4-[3-(methylsulfanyl)propoxy]phenyl}-2,3-dihydro-1-benzofuran-3-yl]amino}-2,3-dihydro-1-benzofuran-3-yl]acetic acid

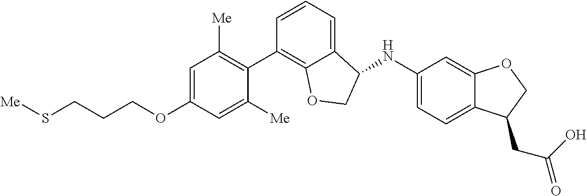

In the same manner as in Example 48, the title compound was obtained as a white solid from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 3-(methylmercapto)propan-1-ol.

¹H NMR (300 MHz, CDCl₃) δ 2.00-2.12 (8H, m), 2.13 (3H, s), 2.54-2.74 (3H, m), 2.79 (1H, dd, J=16.7, 5.3 Hz), 3.72-3.86 (1H, m), 4.06 (2H, t, J=6.1 Hz), 4.20-4.38 (2H, m), 4.67 (1H, dd, J=9.7, 7.4 Hz), 4.74 (1H, t, J=9.1 Hz), 5.22 (1H, dd, J=7.2, 4.2 Hz), 6.10-6.19 (2H, m), 6.68 (2H, s), 6.92-7.06 (3H, m), 7.33 (1H, d, J=5.7 Hz).

MS m/z 520 (M+H)⁺.

### Example 59

### [(3S)-6-{[(3S)-7-{4-[2-(azepan-1-yl)ethoxy]-2,6-dimethylphenyl}-2,3-dihydro-1-benzofuran-3-yl]amino}-2,3-dihydro-1-benzofuran-3-yl]acetic acid

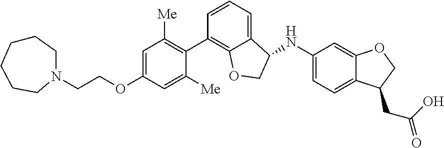

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and N-(2-hydroxyethyl)hexamethyleneimine.

¹H NMR (300 MHz, CDCl₃) δ 1.61-1.74 (4H, m), 1.76-1.91 (4H, m), 2.03 (3H, s), 2.05 (3H, s), 2.47 (1H, dd, J=15.9, 9.1 Hz), 2.62-2.73 (1H, m), 3.09-3.18 (4H, m), 3.20-3.33 (2H, m), 3.71-3.86 (1H, m), 4.21-4.33 (4H, m), 4.61 (1H, dd, J=9.3, 7.4 Hz), 4.74 (1H, t, J=8.9 Hz), 5.17 (1H, dd, J=6.6, 4.4 Hz), 5.99-6.13 (2H, m), 6.66 (2H, s), 6.88-7.04 (3H, m), 7.28-7.36 (1H, m).

MS m/z 557 (M+H)⁺.

### Example 60

### [(3S)-6-{[(3S)-7-(4-{2-[2-(dimethylamino)ethoxy]ethoxy}-2,6-dimethylphenyl)-2,3-dihydro-1-benzofuran-3-yl]amino}-2,3-dihydro-1-benzofuran-3-yl]acetic acid

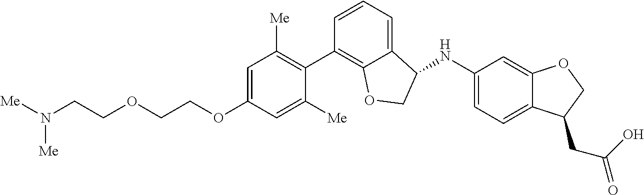

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 2-(2-dimethylaminoethoxy)ethanol.

¹H NMR (300 MHz, CDCl₃) δ 2.02 (3H, s), 2.05 (3H, s), 2.46 (1H, dd, J=15.9, 9.1 Hz), 2.56 (6H, s), 2.68 (1H, dd, J=16.3, 5.3 Hz), 2.86-2.97 (2H, m), 3.70-3.87 (5H, m), 4.08-4.17 (2H, m), 4.22-4.34 (2H, m), 4.62 (1H, t, J=8.5 Hz), 4.76 (1H, t, J=9.1 Hz), 5.18 (1H, dd, J=7.2, 4.5 Hz), 6.04-6.14 (2H, m), 6.67 (2H, s), 6.89-7.04 (3H, m), 7.32 (1H, dd, J=6.8, 1.5 Hz).

MS m/z 547 (M+H)⁺.

### Example 61

### [(3S)-6-{[(3S)-7-{2,6-dimethyl-4-[(5-methylisoxazol-3-yl)methoxy]phenyl}-2,3-dihydro-1-benzofuran-3-yl]amino}-2,3-dihydro-1-benzofuran-3-yl]acetic acid

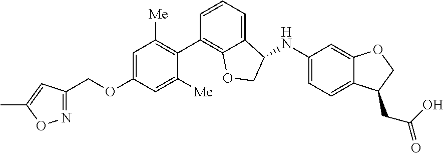

In the same manner as in Example 48, the title compound was obtained as a colorless oil from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 3-hydroxymethyl-5-methylisoxazole.

¹H NMR (300 MHz, CDCl₃) δ 2.04 (3H, s), 2.07 (3H, s), 2.43 (3H, s), 2.56 (1H, dd, J=16.3, 9.1 Hz), 2.77 (1H, dd, J=16.7, 4.9 Hz), 3.69-3.84 (1H, m), 4.20-4.38 (2H, m), 4.61-4.78 (2H, m), 5.10 (2H, s), 5.21 (1H, dd, J=7.0, 4.4 Hz), 6.08-6.18 (3H, m), 6.74 (2H, s), 6.91-7.06 (3H, m), 7.33 (1H, d, J=5.7 Hz).

MS m/z 527 (M+H)⁺.

### Example 62

### [(3S)-6-{[(3S)-7-{2,6-dimethyl-4-[2-(morpholin-4-yl)ethoxy]phenyl}-2,3-dihydro-1-benzofuran-3-yl]amino}-2,3-dihydro-1-benzofuran-3-yl]acetic acid

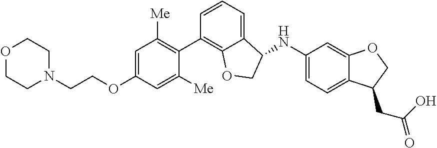

In the same manner as in Example 48, the title compound was obtained as a white solid from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and N-(2-hydroxyethyl)morpholine.

¹H NMR (300 MHz, CDCl₃) δ 2.04 (3H, s), 2.07 (3H, s), 2.57 (1H, dd, J=16.7, 9.5 Hz), 2.63-2.70 (4H, m), 2.76 (1H, dd, J=16.7, 5.3 Hz), 2.85 (2H, t, J=5.7 Hz), 3.70-3.83 (5H, m), 4.14 (2H, t, J=5.5 Hz), 4.21-4.37 (2H, m), 4.65 (1H, dd, J=9.7, 7.4 Hz), 4.74 (1H, t, J=9.1 Hz), 5.20 (1H, dd, J=7.4, 4.4 Hz), 6.08-6.16 (2H, m), 6.68 (2H, s), 6.87-7.06 (3H, m), 7.33 (1H, d, J=5.3 Hz).

MS m/z 545 (M+H)⁺.

### Example 63

### [(3S)-6-{[(3S)-7-{2,6-dimethyl-4-[(3-methyloxetan-3-yl)methoxy]phenyl}-2,3-dihydro-1-benzofuran-3-yl]amino}-2,3-dihydro-1-benzofuran-3-yl]acetic acid

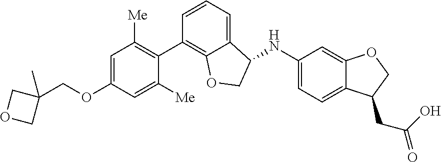

In the same manner as in Example 48, the title compound was obtained as a white solid from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 3-methyl-3-oxetanemethanol.

¹H NMR (300 MHz, CDCl₃) δ 1.43 (3H, s), 2.06 (3H, s), 2.09 (3H, s), 2.61 (1H, dd, J=16.7, 9.1 Hz), 2.80 (1H, dd, J=16.7, 5.3 Hz), 3.73-3.87 (1H, m), 4.03 (2H, s), 4.23-4.38 (2H, m), 4.46 (2H, d, J=6.1 Hz), 4.59-4.81 (4H, m), 5.22 (1H, dd, J=7.0, 4.4 Hz), 6.10-6.22 (2H, m), 6.72 (2H, s), 6.93-7.08 (3H, m), 7.34 (1H, d, J=5.7 Hz).

### Example 64

### [(3S)-6-{[(3S)-7-{2,6-dimethyl-4-[3-(morpholin-4-yl)propoxy]phenyl}-2,3-dihydro-1-benzofuran-3-yl]amino}-2,3-dihydro-1-benzofuran-3-yl]acetic acid

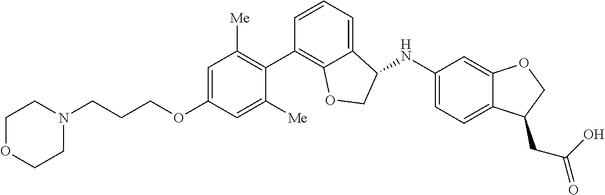

In the same manner as in Example 48, the title compound was obtained as a white solid from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and 3-morpholinopropanol.

¹H NMR (300 MHz, CDCl₃) δ 1.96-2.09 (8H, m), 2.52 (1H, dd, J=16.6, 8.7 Hz), 2.58-2.78 (7H, m), 3.70-3.85 (5H, m), 4.02 (2H, t, J=6.0 Hz), 4.20-4.29 (2H, m), 4.54 (1H, dd, J=9.6, 7.3 Hz), 4.72 (1H, t, J=9.0 Hz), 5.15 (1H, dd, J=7.2, 4.1 Hz), 6.03-6.14 (2H, m), 6.67 (2H, s), 6.90-7.05 (3H, m), 7.32 (1H, dd, J=6.6, 2.1 Hz).

MS m/z 559 (M+H)⁺.

### Example 65

### [(3S)-6-({(3S)-7-[4-(cyclopentylmethoxy)-2,6-dimethylphenyl]-2,3-dihydro-1-benzofuran-3-yl}amino)-2,3-dihydro-1-benzofuran-3-yl]acetic acid

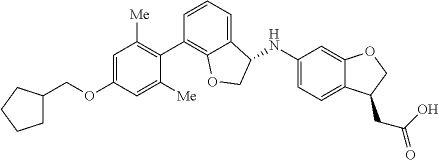

In the same manner as in Example 48, the title compound was obtained as a white solid from methyl [(3S)-6-{[(3S)-7-(4-hydroxy-2,6-dimethylphenyl)-2,3-dihydro-1-benzofuran-3-yl](trifluoroacetyl)amino}-2,3-dihydro-1-benzofuran-3-yl]acetate obtained in the process of obtaining Reference Example 10 and cyclopentanemethanol.

¹H NMR (300 MHz, CDCl₃) δ 1.27-1.46 (2H, m), 1.52-1.70 (4H, m), 1.75-1.95 (2H, m), 2.04 (3H, s), 2.07 (3H, s), 2.29-2.46 (1H, m), 2.54-2.71 (1H, m), 2.80 (1H, dd, J=16.6, 5.3 Hz), 3.73-3.89 (3H, m), 4.20-4.39 (2H, m), 4.67 (1H, dd, J=9.6, 7.3 Hz), 4.74 (1H, t, J=9.0 Hz), 5.22 (1H, dd, J=7.3, 4.3 Hz), 6.10-6.19 (2H, m), 6.63-6.75 (2H, m), 6.92-7.12 (3H, m), 7.28-7.39 (1H, m).

MS m/z 514 (M+H)⁺.

### Example 66

### methyl [(3S)-6-({7-[1-(difluoromethyl)-3,5-dimethyl-1H-pyrazol-4-yl]-2,3-dihydro-1-benzofuran-3-yl}amino)-2,3-dihydro-1-benzofuran-3-yl]acetate

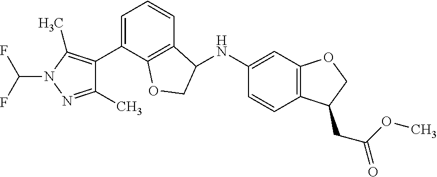

To a mixed solution of methyl {(3S)-6-[(7-bromo-2,3-dihydro-1-benzofuran-3-yl)amino]-2,3-dihydro-1-benzofuran-3-yl}acetate (0.323 g, 0.800 mmol), 1-(difluoromethyl)-3,5-dimethyl-4-(4,4,5,5-tetramethyl-1,3,2-dioxaborolan-2-yl)-1H-pyrazole (0.327 g, 1.20 mmol) and tripotassium phosphate (0.340 g, 1.60 mmol) in toluene (4 mL) and water (1 mL) were added palladium (II) acetate (0.018 g, 0.080 mmol) and 2-dicyclohexylphosphino-2′,6′-dimethoxybiphenyl (0.033 g, 0.080 mmol), and the mixture was stirred at 100° C. for 22 hr under an argon atmosphere. To the reaction mixture were added water and ethyl acetate, and the insoluble material was filtered off through celite. The organic layer of the filtrate was washed with saturated brine, dried over anhydrous sodium sulfate, and concentrated under reduced pressure. The residue was purified by silica gel column chromatography (ethyl acetate:hexane=5:95-40:60) to give the title compound (0.276 g, yield 74%) as a pale-yellow viscous oil.

¹H NMR (300 MHz, CDCl₃) δ 2.20 (3H, s), 2.36 (3H, s), 2.50-2.61 (1H, m), 2.69-2.80 (1H, m), 3.72 (3H, s), 3.73-3.85 (1H, m), 4.03 (1H, d, J=7.3 Hz), 4.25 (1H, dd, J=9.1, 5.9 Hz), 4.41 (1H, dd, J=9.7, 4.2 Hz), 4.68-4.78 (2H, m), 5.18-5.28 (1H, m), 6.10-6.19 (2H, m), 6.95-7.39 (5H, m).

MS m/z 470 (M+H)⁺.

### Example 67

### [(3S)-6-({7-[1-(difluoromethyl)-3,5-dimethyl-1H-pyrazol-4-yl]-2,3-dihydro-1-benzofuran-3-yl}amino)-2,3-dihydro-1-benzofuran-3-yl]acetic acid

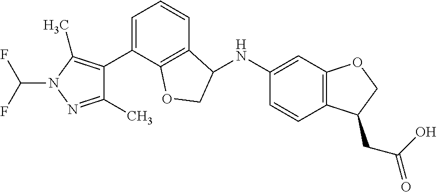

To a mixed solution of methyl [(3S)-6-({7-[1-(difluoromethyl)-3,5-dimethyl-1H-pyrazol-4-yl]-2,3-dihydro-1-benzofuran-3-yl}amino)-2,3-dihydro-1-benzofuran-3-yl]acetate (0.272 g, 0.579 mmol) in methanol (1.2 mL) and tetrahydrofuran (1.2 mL) was added 1 M aqueous sodium hydroxide solution (1.2 mL), and the mixture was stirred at 50° C. for 2 hr. The reaction mixture was concentrated under reduced pressure, and the residue was dissolved in water. The solution was neutralized with 1 M hydrochloric acid, and the precipitated solid was collected by filtration and dried to give the title compound (0.231 g, yield 88%) as a colorless powder.

¹H NMR (300 MHz, CDCl₃) δ 2.20 (3H, s), 2.36 (3H, s), 2.55-2.68 (1H, m), 2.74-2.87 (1H, m), 3.73-3.87 (1H, m), 4.28 (1H, dd, J=9.2, 6.0 Hz), 4.41 (1H, dd, J=9.6, 4.1 Hz), 4.68-4.80 (2H, m), 5.24 (1H, dd, J=7.2, 4.1 Hz), 6.11-6.20 (2H, m), 6.96-7.41 (5H, m).

MS (ESI−) m/z 454 (M−H)⁻.

### Example 68

### methyl [(3S)-6-{[7-(2-methyl-1H-benzimidazol-1-yl)-2,3-dihydro-1-benzofuran-3-yl]amino}-2,3-dihydro-1-benzofuran-3-yl]acetate

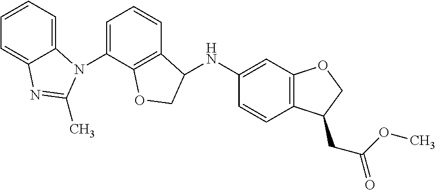

To a mixture of methyl {(3S)-6-[(7-bromo-2,3-dihydro-1-benzofuran-3-yl)amino]-2,3-dihydro-1-benzofuran-3-yl}acetate (0.555 g, 1.37 mmol), o-aminoacetanilide (0.309 g, 2.06 mmol) and cesium carbonate (0.280 g, 2.75 mmol) in toluene (8 ml) were added tris(dibenzylideneacetone)dipalladium (0) (0.063 g, 0.069 mmol) and 4,5-bis(diphenylphosphino)-9,9-dimethylxanthene (0.079 g, 0.137 mmol), and the mixture was stirred at 100° C. for 18 hr under an argon atmosphere. To the reaction mixture were added water and ethyl acetate, and the insoluble material was filtered off through celite. The organic layer of the filtrate was washed with saturated brine, dried over anhydrous magnesium sulfate, and concentrated under reduced pressure. The residue was purified by silica gel column chromatography (ethyl acetate:hexane=40:100-100:0) to give the title compound (56.3 mg, yield 9%) as a brown oil.

¹H NMR (300 MHz, CDCl₃) δ 2.49-2.61 (4H, m), 2.70-2.80 (1H, m), 3.72 (3H, s), 3.74-3.85 (1H, m), 4.06 (1H, d, J=7.6 Hz), 4.26 (1H, dd, J=9.1, 6.1 Hz), 4.37-4.48 (1H, m), 4.70-4.84 (2H, m), 5.26-5.39 (1H, m), 6.13-6.20 (2H, m), 6.99 (1H, d, J=8.3 Hz), 7.04-7.14 (2H, m), 7.16-7.31 (3H, m), 7.51 (1H, dd, J=7.2, 0.8 Hz), 7.71-7.76 (1H, m).

MS m/z 456 (M+H)⁺.

### Example 69

### [(3S)-6-{[7-(2-methyl-1H-benzimidazol-1-yl)-2,3-dihydro-1-benzofuran-3-yl]amino}-2,3-dihydro-1-benzofuran-3-yl]acetic acid

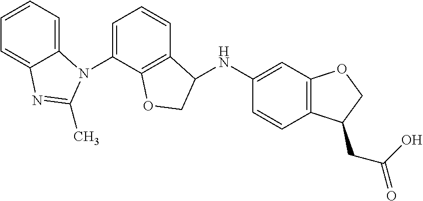

In the same manner as in Example 67, the title compound was obtained as a colorless powder from methyl [(3S)-6-{[7-(2-methyl-1H-benzimidazol-1-yl)-2,3-dihydro-1-benzofuran-3-yl]amino}-2,3-dihydro-1-benzofuran-3-yl]acetate. yield 66%.

¹H NMR (300 MHz, CDCl₃) δ 2.53 and 2.55 (3H, s), 2.57-2.69 (1H, m), 2.77-2.87 (1H, m), 3.78-3.91 (1H, m), 4.31 (1H, dd, J=9.1, 6.1 Hz), 4.37-4.49 (1H, m), 4.70-4.85 (2H, m), 5.27-5.38 (1H, m), 6.12-6.21 (2H, m), 7.01-7.16 (3H, m), 7.17-7.32 (3H, m), 7.52 (1H, d, J=7.3 Hz), 7.77 (1H, d, J=7.9 Hz).

MS m/z 442 (M+H)⁺.

### Example 70

### [(3S)-6-{[7-(2,5-dimethyl-1H-benzimidazol-1-yl)-2,3-dihydro-1-benzofuran-3-yl]amino}-2,3-dihydro-1-benzofuran-3-yl]acetic acid

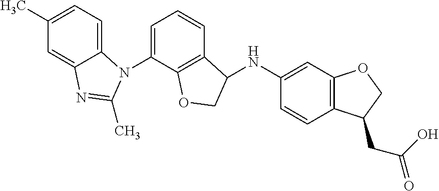

To a mixed solution of methyl [(3S)-6-{[7-(2,5-dimethyl-1H-benzimidazol-1-yl)-2,3-dihydro-1-benzofuran-3-yl](trifluoroacetyl)amino}-2,3-dihydro-1-benzofuran-3-yl]acetate (0.101 g, 0.179 mmol) in methanol (0.5 mL) and tetrahydrofuran (1 mL) was added 1 M aqueous sodium hydroxide solution (0.5 mL), and the mixture was stirred at 50° C. for 2 hr. The reaction mixture was concentrated under reduced pressure, and the residue was dissolved in water. The solution was neutralized with 1 M hydrochloric acid, and the precipitated solid was collected by filtration and dried to give the title compound (56.4 mg, yield 69%) as a colorless powder.

¹H NMR (300 MHz, CDCl₃) δ 2.47 (3H, s), 2.52 (3H, d, J=5.7 Hz), 2.57-2.69 (1H, m), 2.76-2.87 (1H, m), 3.79-3.91 (1H, m), 4.32 (1H, dd, J=9.3, 6.2 Hz), 4.36-4.48 (1H, m), 4.70-4.84 (2H, m), 5.27-5.37 (1H, m), 6.12-6.20 (2H, m), 6.93-7.14 (4H, m), 7.23-7.30 (1H, m), 7.51 (1H, d, J=6.8 Hz), 7.56 (1H, s).

MS m/z 456 (M+H)⁺.

### Example 71

### [(3S)-6-{[7-(6-fluoro-2-methyl-1H-benzimidazol-1-yl)-2,3-dihydro-1-benzofuran-3-yl]amino}-2,3-dihydro-1-benzofuran-3-yl]acetic acid

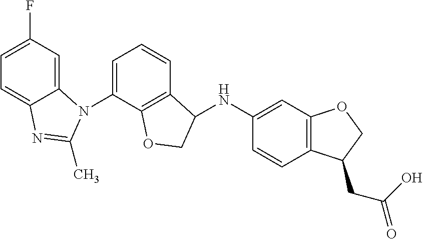

In the same manner as in Example 70, the title compound was obtained as a colorless powder from methyl [(3S)-6-{[7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate. yield 76%.

¹H NMR (300 MHz, CDCl₃) δ 2.51 (3H, d, J=6.4 Hz), 2.57-2.69 (1H, m), 2.76-2.87 (1H, m), 3.77-3.90 (1H, m), 4.31 (1H, dd, J=9.1, 6.1 Hz), 4.44 (1H, td, J=10.2, 4.5 Hz), 4.73-4.85 (2H, m), 5.28-5.40 (1H, m), 6.13-6.22 (2H, m), 6.78 (1H, ddd, J=13.1, 8.5, 2.7 Hz), 6.96-7.16 (3H, m), 7.23-7.31 (1H, m), 7.53 (1H, d, J=7.2 Hz), 7.68 (1H, dd, J=8.7, 4.9 Hz).

MS m/z 460 (M+H)⁺.

### Example 72

### [(3S)-6-{[(3S)-7-(6-fluoro-2-methyl-1H-benzimidazol-1-yl)-2,3-dihydro-1-benzofuran-3-yl]amino}-2,3-dihydro-1-benzofuran-3-yl]acetic acid

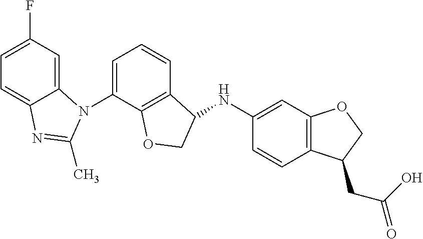

To a mixed solution of methyl [(3S)-6-{[(3S)-7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate (0.305 g, 0.536 mmol) in methanol (2 mL) and tetrahydrofuran (4 mL) was added 1 M aqueous sodium hydroxide solution (2 mL), and the mixture was stirred at 50° C. for 2 hr. The reaction mixture was diluted with water, weak acidified with 1 M hydrochloric acid, and extracted with ethyl acetate. The extract was washed with saturated brine, dried over anhydrous sodium sulfate, and concentrated under reduced pressure. The precipitated crystals were recrystallized from heptane-ethyl acetate to give the title compound (0.206 g, yield 84%) as colorless crystals.

¹H NMR (300 MHz, DMSO-d₆) δ 2.37-2.51 (4H, m), 2.60-2.71 (1H, m), 3.56-3.69 (1H, m), 4.08-4.16 (1H, m), 4.22-4.30 (1H, m), 4.58-4.67 (1H, m), 4.75-4.87 (1H, m), 5.33-5.44 (1H, m), 6.15-6.26 (3H, m), 6.86 (1H, ddd, J=11.5, 9.0, 2.4 Hz), 6.97 (1H, dd, J=8.1, 2.4 Hz), 7.01-7.17 (2H, m), 7.41-7.47 (1H, m), 7.53 (1H, d, J=7.5 Hz), 7.61 (1H, dd, J=8.9, 5.1 Hz), 12.31 (1H, br s).

MS m/z 460 (M+H)⁺.

### Example 73

### [(3S)-6-{[(3R)-7-(6-fluoro-2-methyl-1H-benzimidazol-1-yl)-2,3-dihydro-1-benzofuran-3-yl]amino}-2,3-dihydro-1-benzofuran-3-yl]acetic acid

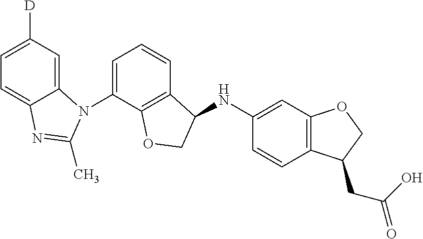

To a mixed solution of methyl [(3S)-6-{[(3R)-7-(6-fluoro-2-methyl-1H-benzimidazol-1-yl)-2,3-dihydro-1-benzofuran-3-yl](trifluoroacetyl)amino}-2,3-dihydro-1-benzofuran-3-yl]acetate (0.314 g, 0.551 mmol) in methanol (2 mL) and tetrahydrofuran (4 mL) was added 1 M aqueous sodium hydroxide solution (2 mL), and the mixture was stirred at 50° C. for 2 hr. The reaction mixture was diluted with water, weak acidified with 1 M hydrochloric acid, and extracted with ethyl acetate. The extract was washed with saturated brine, dried over anhydrous sodium sulfate, and concentrated under reduced pressure. The residue was washed with ethanol-water to give the title compound (0.257 g, quantitative) as an off-white powder.

¹H NMR (300 MHz, DMSO-d₆) δ 2.38-2.50 (4H, m), 2.60-2.71 (1H, m), 3.55-3.69 (1H, m), 4.08-4.16 (1H, m), 4.26 (1H, ddd, J=9.4, 4.7, 1.7 Hz), 4.62 (1H, td, J=9.0, 1.5 Hz), 4.75-4.87 (1H, m), 5.34-5.44 (1H, m), 6.14-6.26 (3H, m), 6.85-6.94 (1H, m), 6.97 (1H, dd, J=7.9, 2.6 Hz), 7.03-7.18 (2H, m), 7.42-7.48 (1H, m), 7.54 (1H, d, J=7.2 Hz), 7.63 (1H, dd, J=8.7, 4.9 Hz).

MS m/z 460 (M+H)⁺.

### Example 74

### [(3S)-6-({(3S)-7-[(5-fluoropyridin-2-yl)(propyl)amino]-2,3-dihydro-1-benzofuran-3-yl}amino)-2,3-dihydro-1-benzofuran-3-yl]acetic acid

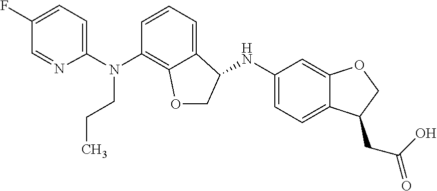

To a solution of methyl {(3S)-6-[{(3S)-7-[(5-fluoropyridin-2-yl)amino]-2,3-dihydro-1-benzofuran-3-yl}(trifluoroacetyl)amino]-2,3-dihydro-1-benzofuran-3-yl}acetate (0.186 g, 0.351 mmol) and n-propyl iodide (89.0 mg, 0.526 mmol) in N,N-dimethylformamide (2 mL) was added sodium hydride (60% in oil, 16.8 mg, 0.421 mmol) at 0° C., and the mixture was stirred at room temperature for 1.5 hr. The reaction mixture was poured into water, and the mixture was extracted with ethyl acetate. The extract was dried over anhydrous sodium sulfate, and concentrated under reduced pressure. The residue was purified by silica gel column chromatography (hexane:ethyl acetate=95:5-70:30) to give a mixture (0.125 g) of methyl {(3S)-6-[{(3S)-7-[(5-fluoropyridin-2-yl)(propyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(5-fluoropyridin-2-yl)(propyl)amino]-2,3-dihydro-1-benzofuran-3-yl}amino)-2,3-dihydro-1-benzofuran-3-yl]acetate as a pale-yellow oil. To a mixed solution of the mixture (0.125 g) obtained above in tetrahydrofuran (2 mL) and methanol (1 mL) was added 1 M aqueous sodium hydroxide solution (0.787 mL), and the mixture was stirred at room temperature for 2 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure to give the title compound (0.109 g, yield 67%, 2 steps) as a pale-yellow solid.

¹H NMR (300 MHz, CDCl₃) δ 0.91 (3H, t, J=7.2 Hz), 1.55-1.74 (2H, m), 2.59 (1H, dd, J=16.7, 9.1 Hz), 2.79 (1H, dd, J=16.7, 5.3 Hz), 3.71-3.89 (3H, m), 4.27 (1H, dd, J=9.1, 6.1 Hz), 4.37 (1H, dd, J=9.7, 4.4 Hz), 4.64-4.82 (2H, m), 5.21 (1H, dd, J=7.6, 4.2 Hz), 6.09-6.19 (2H, m), 6.27 (1H, dd, J=9.3, 3.6 Hz), 6.90-7.20 (4H, m), 7.21-7.32 (1H, m), 8.03 (1H, d, J=3.4 Hz).

MS m/z 464 (M+H)⁺.

### Example 75

### sodium [(3S)-6-({(3S)-7-[(5-fluoropyridin-2-yl)(propyl)amino]-2,3-dihydro-1-benzofuran-3-yl}amino)-2,3-dihydro-1-benzofuran-3-yl]acetate

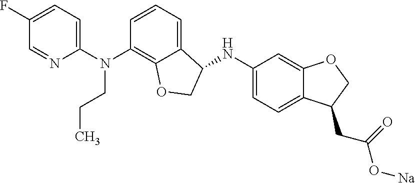

To a suspension of [(3S)-6-({(3S)-7-[(5-fluoropyridin-2-yl)(propyl)amino]-2,3-dihydro-1-benzofuran-3-yl}amino)-2,3-dihydro-1-benzofuran-3-yl]acetic acid (75.7 mg, 0.163 mmol) in water (1 mL) was added 1 M aqueous sodium hydroxide solution (0.163 mL), and the mixture was stirred at room temperature. After dissolution of insoluble material, acetonitrile was added and the mixture was concentrated under reduced pressure to give the title compound (75.7 mg, yield 95%) as a pale-yellow solid.

¹H NMR (300 MHz, DMSO-d₆) δ 0.84 (3H, t, J=7.4 Hz), 1.47-1.64 (2H, m), 1.98 (1H, dd, J=15.0, 10.0 Hz), 2.32 (1H, dd, J=15.0, 4.7 Hz), 3.45-3.63 (1H, m), 3.68-3.85 (2H, m), 4.01-4.12 (1H, m), 4.17 (1H, dd, J=9.5, 4.9 Hz), 4.60 (1H, t, J=8.9 Hz), 4.72 (1H, t, J=8.7 Hz), 5.19-5.34 (1H, m), 6.00 (1H, d, J=8.0 Hz), 6.06-6.29 (3H, m), 6.84-7.03 (2H, m), 7.16 (1H, d, J=7.6 Hz), 7.22-7.42 (2H, m), 8.08 (1H, d, J=3.0 Hz).

MS m/z 464 (M+H)⁺ (as free form).

### Example 76

### methyl {(3S)-6-[{(3S)-7-[4,6-dimethyl-2-(morpholin-4-yl)pyrimidin-5-yl]-2,3-dihydro-1-benzofuran-3-yl}amino]-2,3-dihydro-1-benzofuran-3-yl}acetate

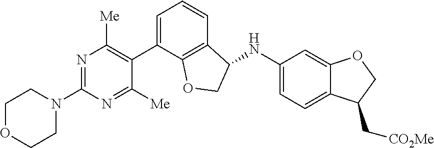

A solution of (3S)-7-(4,6-dimethyl-2-morpholin-4-ylpyrimidin-5-yl)-2,3-dihydro-1-benzofuran-3-amine (55.7 mg, 0.171 mmol), methyl [(3S)-6-{[(trifluoromethyl)sulfonyl]oxy}-2,3-dihydro-1-benzofuran-3-yl]acetate (63.9 mg, 0.188 mmol) obtained in the process of obtaining Reference Example 1 and cesium carbonate (0.167 g, 0.512 mmol) in toluene (3 mL) was substituted with argon, and tris(dibenzylideneacetone)dipalladium (0) (6.25 mg, 0.00683 mmol) and 2-dicyclohexylphosphino-2′,4′,6′-triisopropylbiphenyl (6.51 mg, 0.0140 mmol) were added. The reaction mixture was stirred under an argon atmosphere at 100° C. for 16 hr. The reaction mixture was cooled, insoluble material was filtered off through celite, and the filtrate was concentrated under reduced pressure. The residue was purified by silica gel column chromatography (hexane:ethyl acetate=95:5-50:50) to give the title compound (69.8 mg, yield 79%) as a yellow oil.

¹H NMR (300 MHz, DMSO-d₆) δ 2.05 (3H, s), 2.09 (3H, s), 2.50-2.61 (1H, m), 2.72 (1H, dd, J=16.6, 6.0 Hz), 3.56-3.77 (12H, m), 4.08-4.21 (2H, m), 4.61 (1H, t, J=8.9 Hz), 4.67-4.78 (1H, m), 5.21-5.34 (1H, m), 6.09-6.24 (3H, m), 6.91 (1H, d, J=8.7 Hz), 6.93-7.02 (1H, m), 7.03-7.11 (1H, m), 7.34 (1H, d, J=6.8 Hz).

### Example 77

### [(3S)-6-({(3S)-7-[4,6-dimethyl-2-(morpholin-4-yl)pyrimidin-5-yl]-2,3-dihydro-1-benzofuran-3-yl}amino)-2,3-dihydro-1-benzofuran-3-yl]acetic acid

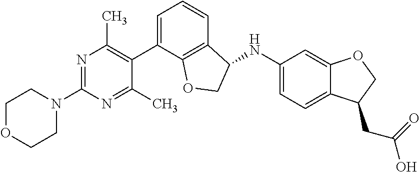

To a mixed solution of methyl {(3S)-6-[{(3S)-7-[4,6-dimethyl-2-(morpholin-4-yl)pyrimidin-5-yl]-2,3-dihydro-1-benzofuran-3-yl}(trifluoroacetyl)amino]-2,3-dihydro-1-benzofuran-3-yl}acetate (3.90 g, 6.37 mmol) in tetrahydrofuran (20 mL) and methanol (10 mL) was added 1 M aqueous sodium hydroxide solution (19.1 mL), and the mixture was stirred at 50° C. for 3 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure. The residue was purified by silica gel column chromatography (hexane:ethyl acetate=70:30-0:100) and crystallized from methanol. The obtained crystals were recrystallized from ethanol-water to give the title compound (2.26 g, yield 71%) as white crystals.

¹H NMR (300 MHz, DMSO-d₆) δ 2.05 (3H, s), 2.09 (3H, s), 2.44 (1H, dd, J=16.6, 9.0 Hz), 2.65 (1H, dd, J=16.6, 5.7 Hz), 3.53-3.76 (9H, m), 4.05-4.20 (2H, m), 4.62 (1H, t, J=9.0 Hz), 4.67-4.78 (1H, m), 5.22-5.35 (1H, m), 6.06-6.24 (3H, m), 6.90-7.02 (2H, m), 7.03-7.11 (1H, m), 7.34 (1H, d, J=7.2 Hz), 12.31 (1H, br s).

MS m/z 503 (M+H)⁺.

melting point: 168° C.

elemental analysis value for C₂₈H₃₀N₄O₅

Calculated: C, 66.92; H, 6.02; N, 11.15.

Found: C, 66.84; H, 6.12; N, 11.08.

Alternatively, to a mixed solution of methyl {(3S)-6-[{(3S)-7-[4,6-dimethyl-2-(morpholin-4-yl)pyrimidin-5-yl]-2,3-dihydro-1-benzofuran-3-yl}amino]-2,3-dihydro-1-benzofuran-3-yl}acetate (67.8 mg, 0.131 mmol) in tetrahydrofuran (1.5 mL) and methanol (0.75 mL) was added 1 M aqueous sodium hydroxide solution (0.394 mL), and the mixture was stirred at 50° C. for 2 hr. The reaction mixture was neutralized with 1 M hydrochloric acid (0.394 mL), diluted with brine, and extracted with ethyl acetate. The extract was washed with saturated brine, dried over anhydrous sodium sulfate, and concentrated under reduced pressure. The residue was purified by silica gel column chromatography (hexane:ethyl acetate:methanol=90:10:0-0:100:0-0:85:15), and crystallized from methanol-hexane to give the title compound (32.5 mg, yield 49%) as pale-yellow crystals.

### Example 77-1

### Hydrate of [(3S)-6-({(3S)-7-[4,6-dimethyl-2-(morpholin-4-yl)pyrimidin-5-yl]-2,3-dihydro-1-benzofuran-3-yl}amino)-2,3-dihydro-1-benzofuran-3-yl]acetic acid

To a mixed solution of methyl {(3S)-6-[{(3S)-7-[4,6-dimethyl-2-(morpholin-4-yl)pyrimidin-5-yl]-2,3-dihydro-1-benzofuran-3-yl}(trifluoroacetyl)amino]-2,3-dihydro-1-benzofuran-3-yl}acetate (2.24 g, 3.66 mmol) in tetrahydrofuran (14 mL) and methanol (7 mL) was added 1 M aqueous sodium hydroxide solution (11.0 mL), and the mixture was stirred at 50° C. for 2 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure. The residue was crystallized from methanol-diisopropyl ether to give a first crystal (1.10 g). Mother liquor was concentrated, and the obtained solid was triturated with methanol. The solid was collected by filtration, and dried to give a second crystal (0.460 g). Furthermore, a similar operation was repeated to give a third crystal (0.109 g). The first to third crystals were combined and recrystallized from ethanol to give the title compound (1.30 g, yield 71%) as pale-yellow crystals.

¹H NMR (300 MHz, DMSO-d₆) δ 2.05 (3H, s), 2.09 (3H, s), 2.44 (1H, dd, J=16.6, 9.0 Hz), 2.65 (1H, dd, J=16.6, 5.7 Hz), 3.53-3.76 (9H, m), 4.05-4.20 (2H, m), 4.62 (1H, t, J=9.0 Hz), 4.67-4.78 (1H, m), 5.22-5.35 (1H, m), 6.06-6.24 (3H, m), 6.90-7.02 (2H, m), 7.03-7.11 (1H, m), 7.34 (1H, d, J=7.2 Hz), 12.31 (1H, br s).

MS m/z 503 (M+H)⁺.

melting point: 133° C.

elemental analysis value for C₂₈H₃₀N₄O₅.0.5H₂O

Calculated: C, 65.74; H, 6.11; N, 10.95.

Found: C, 65.79; H, 6.18; N, 10.96.

### Example 77-2

### [(3S)-6-({(3S)-7-[4,6-dimethyl-2-(morpholin-4-yl)pyrimidin-5-yl]-2,3-dihydro-1-benzofuran-3-yl}amino)-2,3-dihydro-1-benzofuran-3-yl]acetic acid

To a mixed solution of methyl {(3S)-6-[{(3S)-7-[4,6-dimethyl-2-(morpholin-4-yl)pyrimidin-5-yl]-2,3-dihydro-1-benzofuran-3-yl}(trifluoroacetyl)amino]-2,3-dihydro-1-benzofuran-3-yl}acetate (0.191 g, 0.312 mmol) in tetrahydrofuran (2 mL) and methanol (1 mL) was added 1 M aqueous sodium hydroxide solution (0.937 mL), and the mixture was stirred at 50° C. for 2 hr. The reaction mixture was neutralized with 1 M hydrochloric acid, diluted with brine, and extracted with ethyl acetate. The extract was dried over anhydrous sodium sulfate, and concentrated under reduced pressure. The residue was purified by silica gel column chromatography (hexane:ethyl acetate=90:10-20:80) to give the title compound (0.122 g, yield 78%) as a white amorphous solid.

¹H NMR (300 MHz, CDCl₃) δ 2.14 (3H, s), 2.17 (3H, s), 2.61 (1H, dd, J=16.6, 9.0 Hz), 2.80 (1H, dd, J=16.6, 5.3 Hz), 3.71-3.90 (9H, m), 4.27 (1H, dd, J=9.4, 6.0 Hz), 4.36 (1H, dd, J=9.6, 4.3 Hz), 4.64-4.80 (2H, m), 5.23 (1H, dd, J=7.3, 4.3 Hz), 6.10-6.21 (2H, m), 6.93-7.07 (3H, m), 7.32-7.40 (1H, m).

MS m/z 503 (M+H)⁺.

### Example 78

### sodium [(3S)-6-({(3S)-7-[4,6-dimethyl-2-(morpholin-4-yl)pyrimidin-5-yl]-2,3-dihydro-1-benzofuran-3-yl}amino)-2,3-dihydro-1-benzofuran-3-yl]acetate

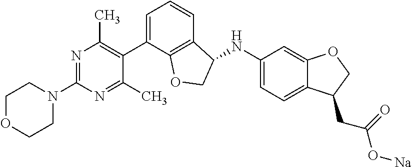

To a suspension of [(3S)-6-({(3S)-7-[4,6-dimethyl-2-(morpholin-4-yl)pyrimidin-5-yl]-2,3-dihydro-1-benzofuran-3-yl}amino)-2,3-dihydro-1-benzofuran-3-yl]acetic acid (87.3 mg, 0.174 mmol) in water (1 mL) was added 1 M aqueous sodium hydroxide solution (0.174 mL), and the mixture was stirred at room temperature. After dissolution of insoluble material, acetonitrile was added and the mixture was concentrated under reduced pressure to give the title compound (90.2 mg, yield 99%) as a beige solid.

¹H NMR (300 MHz, DMSO-d₆) δ 1.92-2.13 (7H, m), 2.33 (1H, dd, J=14.8, 4.9 Hz), 3.47-3.60 (1H, m), 3.60-3.78 (8H, m), 4.07 (1H, dd, J=8.7, 7.2 Hz), 4.16 (1H, dd, J=9.5, 4.9 Hz), 4.60 (1H, t, J=8.9 Hz), 4.72 (1H, t, J=8.5 Hz), 5.20-5.33 (1H, m), 5.99 (1H, d, J=7.6 Hz), 6.08-6.20 (2H, m), 6.87-7.01 (2H, m), 7.03-7.10 (1H, m), 7.34 (1H, d, J=7.2 Hz).

MS m/z 503 (M+H)⁺ (as free form).

### Example 79

### [(3S)-6-({(3S)-7-[4,6-dimethyl-2-(pyrrolidin-1-yl)pyrimidin-5-yl]-2,3-dihydro-1-benzofuran-3-yl}amino)-2,3-dihydro-1-benzofuran-3-yl]acetic acid

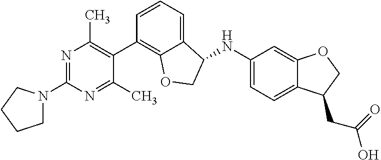

To a mixed solution of methyl [(3S)-6-{[(3S)-7-(4,6-dimethyl-2-pyrrolidin-1-ylpyrimidin-5-yl)-2,3-dihydro-1-benzofuran-3-yl](trifluoroacetyl)amino}-2,3-dihydro-1-benzofuran-3-yl]acetate (1.45 g, 2.43 mmol) in tetrahydrofuran (10 mL) and methanol (5 mL) was added 1 M aqueous sodium hydroxide solution (7.28 mL), and the mixture was stirred at 50° C. for 2 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure. The residue was crystallized from methanol, and the obtained crystals were recrystallized from methanol to give the title compound (0.908 g, yield 77%) as a pale-yellow solid.

¹H NMR (300 MHz, CDCl₃) δ 1.91-2.01 (4H, m), 2.14 (3H, s), 2.17 (3H, s), 2.61 (1H, dd, J=16.6, 9.0 Hz), 2.80 (1H, dd, J=16.6, 5.3 Hz), 3.54-3.66 (4H, m), 3.73-3.86 (1H, m), 4.27 (1H, dd, J=9.2, 5.8 Hz), 4.35 (1H, dd, J=9.6, 4.3 Hz), 4.64-4.80 (2H, m), 5.23 (1H, dd, J=7.3, 4.3 Hz), 6.10-6.19 (2H, m), 6.93-7.07 (3H, m), 7.31-7.37 (1H, m).

MS m/z 487 (M+H)⁺.

melting point: 169° C.

elemental analysis value for C₂₈H₃₀N₄O₄.0.2H₂O

Calculated: C, 68.61; H, 6.25; N, 11.43.

Found: C, 68.67; H, 6.27; N, 11.36.

### Example 80

### sodium [(3S)-6-({(3S)-7-[4,6-dimethyl-2-(pyrrolidin-1-yl)pyrimidin-5-yl]-2,3-dihydro-1-benzofuran-3-yl}amino)-2,3-dihydro-1-benzofuran-3-yl]acetate

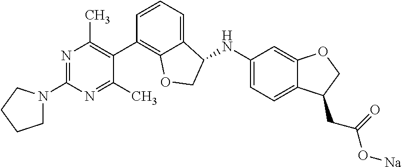

To a solution of [(3S)-6-({(3S)-7-[4,6-dimethyl-2-(pyrrolidin-1-yl)pyrimidin-5-yl]-2,3-dihydro-1-benzofuran-3-yl}amino)-2,3-dihydro-1-benzofuran-3-yl]acetic acid (0.105 mg, 0.215 mmol) in methanol (1 mL) was added 1 M aqueous sodium hydroxide solution (0.251 mL). The mixture was stirred at room temperature for 1 min, acetonitrile was added and the mixture was concentrated under reduced pressure to give the title compound (99.6 mg, yield 91%) as a pale-yellow solid.

¹H NMR (300 MHz, DMSO-d₆) δ 1.82-2.13 (11H, m), 2.34 (1H, dd, J=15.1, 4.9 Hz), 3.42-3.65 (5H, m), 4.01-4.22 (2H, m), 4.61 (1H, t, J=8.9 Hz), 4.71 (1H, t, J=8.7 Hz), 5.18-5.35 (1H, m), 5.99 (1H, d, J=7.9 Hz), 6.09-6.19 (2H, m), 6.87-7.09 (3H, m), 7.33 (1H, d, J=6.8 Hz).

MS m/z 487 (M+H)⁺ (as free form).

### Example 81

### [(3S)-6-({(3S)-7-[ethyl(phenyl)amino]-2,3-dihydro-1-benzofuran-3-yl}amino)-2,3-dihydro-1-benzofuran-3-yl]acetic acid

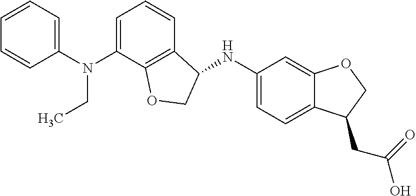

To a mixed solution of methyl {(3S)-6-[{(3S)-7-[ethyl(phenyl)amino]-2,3-dihydro-1-benzofuran-3-yl}(trifluoroacetyl)amino]-2,3-dihydro-1-benzofuran-3-yl}acetate (71.9 mg, 0.133 mmol) in tetrahydrofuran (1 mL) and methanol (0.5 mL) was added 1 M aqueous sodium hydroxide solution (0.399 mL), and the mixture was stirred at 50° C. for 2 hr. The reaction mixture was concentrated under reduced pressure, and water was added to dissolve the residue, and 1 M hydrochloric acid (0.266 mL) was slowly added. The obtained precipitate was collected by filtration, washed with water, and dried to give the title compound (54.3 mg, yield 95%) as a pale-yellow solid.

¹H NMR (300 MHz, CDCl₃) δ 1.21 (3H, t, J=7.2 Hz), 2.57 (1H, dd, J=16.6, 9.0 Hz), 2.76 (1H, dd, J=16.6, 5.3 Hz), 3.66-3.85 (3H, m), 4.25 (1H, dd, J=9.4, 6.0 Hz), 4.34 (1H, dd, J=9.6, 4.3 Hz), 4.62-4.77 (2H, m), 5.17 (1H, dd, J=7.2, 4.1 Hz), 6.06-6.16 (2H, m), 6.68-6.78 (3H, m), 6.89 (1H, t, J=7.5 Hz), 6.97 (1H, d, J=8.7 Hz), 7.08-7.22 (4H, m).

MS m/z 429 (M−H)⁻.

### Example 82

### [(3S)-6-({(3S)-7-[(4-methylpyridin-2-yl)(propyl)amino]-2,3-dihydro-1-benzofuran-3-yl}amino)-2,3-dihydro-1-benzofuran-3-yl]acetic acid

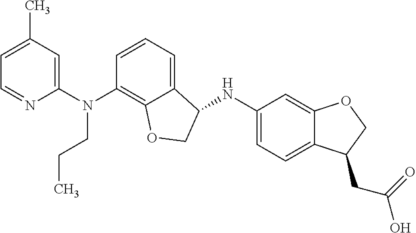

To a solution of methyl {(3S)-6-[{(3S)-7-[(4-methylpyridin-2-yl)amino]-2,3-dihydro-1-benzofuran-3-yl}(trifluoroacetyl)amino]-2,3-dihydro-1-benzofuran-3-yl}acetate (1.98 g, 3.75 mmol) and n-propyl iodide (0.549 mL, 5.63 mmol) in N,N-dimethylformamide (15 mL) was added sodium hydride (60% in oil, 0.180 g, 4.50 mmol) at 0° C., and the mixture was stirred at room temperature for 1 hr. The reaction mixture was poured into aqueous ammonium chloride solution, and the mixture was extracted with ethyl acetate. The extract was washed with water, dried over anhydrous sodium sulfate, and concentrated under reduced pressure. The residue was purified by silica gel column chromatography (hexane:ethyl acetate=100:0-70:30) to give a mixture (1.76 g) of methyl {(3S)-6-[{(3S)-7-[(4-methylpyridin-2-yl)(propyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(4-methylpyridin-2-yl)(propyl)amino]-2,3-dihydro-1-benzofuran-3-yl}amino)-2,3-dihydro-1-benzofuran-3-yl]acetate as a white non-crystalline powder. To a mixed solution of the mixture (1.76 g) obtained above in tetrahydrofuran (14 mL) and methanol (7 mL) was added 1 M aqueous sodium hydroxide solution (9.25 mL), and the mixture was stirred at 50° C. for 2 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure. The residue was purified by silica gel column chromatography (hexane:ethyl acetate=100:0-70:30) and crystallized from 90%.ethanol-water to give the title compound (1.23 g, yield 71%, 2 steps) as a white solid.

¹H NMR (300 MHz, DMSO-d₆) δ 0.84 (3H, t, J=7.4 Hz), 1.45-1.63 (2H, m), 2.08 (3H, s), 2.44 (1H, dd, J=16.7, 9.1 Hz), 2.65 (1H, dd, J=16.3, 5.7 Hz), 3.53-3.69 (1H, m), 3.70-3.85 (2H, m), 4.05-4.21 (2H, m), 4.62 (1H, t, J=8.9 Hz), 4.73 (1H, t, J=8.5 Hz), 5.22-5.35 (1H, m), 6.04 (1H, s), 6.10-6.25 (3H, m), 6.44 (1H, d, J=4.9 Hz), 6.88-7.03 (2H, m), 7.14 (1H, d, J=6.8 Hz), 7.28 (1H, d, J=7.2 Hz), 7.96 (1H, d, J=5.3 Hz), 12.32 (1H, br s).

MS m/z 460 (M+H)⁺.

melting point: 146° C.

elemental analysis value for C₂₇H₂₉N₃O₄

### Calculated: C, 70.57; H, 6.36; N, 9.14.

Found: C, 70.47; H, 6.34; N, 9.06.

### Example 83

### [(3S)-6-{[(3S)-7-(pyridin-2-ylamino)-2,3-dihydro-1-benzofuran-3-yl]amino}-2,3-dihydro-1-benzofuran-3-yl]acetic acid

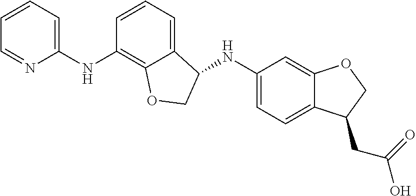

A solution of methyl [(3S)-6-{[(3S)-7-bromo-2,3-dihydro-1-benzofuran-3-yl](trifluoroacetyl)amino}-2,3-dihydro-1-benzofuran-3-yl]acetate (0.176 g, 0.422 mmol), pyridin-2-amine (39.7 mg, 0.422 mmol) and cesium carbonate (0.229 g, 0.704 mmol) in toluene (4 mL) was substituted with argon, and tris(dibenzylideneacetone)dipalladium (0) (12.9 mg, 0.0140 mmol) and 4,5-bis(diphenylphosphino)-9,9-dimethylxanthene (16.3 mg, 0.0280 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-65:35) to give methyl [(3S)-6-{[(3S)-7-(pyridin-2-ylamino)-2,3-dihydro-1-benzofuran-3-yl](trifluoroacetyl)amino}-2,3-dihydro-1-benzofuran-3-yl]acetate (49.5 mg, yield 27%) and methyl [(3S)-6-{[(3S)-7-(pyridin-2-ylamino)-2,3-dihydro-1-benzofuran-3-yl]amino}-2,3-dihydro-1-benzofuran-3-yl]acetate (15.0 mg, yield 10%) each as a brown oil. To a mixed solution of methyl [(3S)-6-{[(3S)-7-(pyridin-2-ylamino)-2,3-dihydro-1-benzofuran-3-yl]amino}-2,3-dihydro-1-benzofuran-3-yl]acetate (15.0 mg, 0.0360 mmol) obtained above in tetrahydrofuran (0.5 mL) and methanol (0.25 mL) was added 1 M aqueous sodium hydroxide solution (0.108 mL), and the mixture was stirred at 50° C. for 1 hr. The reaction mixture was concentrated under reduced pressure, and water was added to dissolve the residue, and 1 M hydrochloric acid (0.108 mL) was slowly added. The obtained precipitate was collected by filtration, washed with water, and dried to give the title compound (13.1 mg, yield 90%) as a pale-yellow solid.

¹H NMR (300 MHz, CDCl₃) δ 2.59 (1H, dd, J=16.2, 8.3 Hz), 2.75 (1H, dd, J=16.2, 6.4 Hz), 3.74-3.91 (1H, m), 4.25 (1H, dd, J=9.0, 6.4 Hz), 4.46 (1H, dd, J=9.8, 4.1 Hz), 4.68-4.82 (2H, m), 5.17-5.26 (1H, m), 6.04-6.17 (2H, m), 6.70-6.86 (2H, m), 6.88-7.00 (1H, m), 7.03-7.15 (2H, m), 7.50-7.61 (2H, m), 8.03-8.13 (1H, m).

MS m/z 404 (M+H)⁺.

### Example 84

### [(3S)-6-({(3S)-7-[(4-methylpyridin-2-yl)amino]-2,3-dihydro-1-benzofuran-3-yl}amino)-2,3-dihydro-1-benzofuran-3-yl]acetic acid

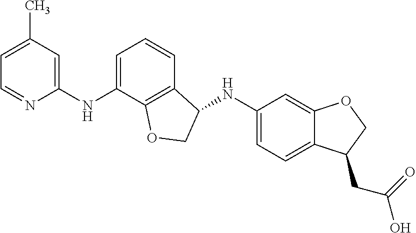

A solution of methyl [(3S)-6-{[(3S)-7-bromo-2,3-dihydro-1-benzofuran-3-yl](trifluoroacetyl)amino}-2,3-dihydro-1-benzofuran-3-yl]acetate (0.203 g, 0.487 mmol), 4-methylpyridin-2-amine (52.6 mg, 0.487 mmol) and cesium carbonate (0.264 g, 0.811 mmol) in toluene (4 mL) was substituted with argon, and tris(dibenzylideneacetone)dipalladium (0) (14.9 mg, 0.0160 mmol) and 4,5-bis(diphenylphosphino)-9,9-dimethylxanthene (18.8 mg, 0.0320 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-65:35) to give methyl {(3S)-6-[{(3S)-7-[(4-methylpyridin-2-yl)amino]-2,3-dihydro-1-benzofuran-3-yl}(trifluoroacetyl)amino]-2,3-dihydro-1-benzofuran-3-yl}acetate (55.5 mg, yield 26%) and methyl [(3S)-6-({(3S)-7-[(4-methylpyridin-2-yl)amino]-2,3-dihydro-1-benzofuran-3-yl}amino)-2,3-dihydro-1-benzofuran-3-yl]acetate (24.9 mg, yield 14%) each as a brown oil. To a mixed solution of methyl [(3S)-6-({(3S)-7-[(4-methylpyridin-2-yl)amino]-2,3-dihydro-1-benzofuran-3-yl}amino)-2,3-dihydro-1-benzofuran-3-yl]acetate (24.9 mg, 0.0580 mmol) obtained above in tetrahydrofuran (0.5 mL) and methanol (0.25 mL) was added 1 M aqueous sodium hydroxide solution (0.173 mL), and the mixture was stirred at 50° C. for 1 hr. The reaction mixture was concentrated under reduced pressure, water was added to dissolve the residue, and 1 M hydrochloric acid was slowly added to neutralize the mixture. The obtained precipitate was collected by filtration, washed with water, and dried to give the title compound (21.3 mg, yield 88%) as a pale-yellow solid.

¹H NMR (300 MHz, CDCl₃) δ 2.28 (3H, s), 2.52 (1H, dd, J=16.4, 9.6 Hz), 2.74 (1H, dd, J=16.2, 5.3 Hz), 3.71-3.86 (1H, m), 4.27 (1H, dd, J=9.0, 6.4 Hz), 4.46 (1H, dd, J=9.6, 4.0 Hz), 4.70-4.80 (2H, m), 5.21 (1H, dd, J=7.2, 4.1 Hz), 6.10-6.18 (2H, m), 6.57-6.64 (2H, m), 6.89-7.10 (3H, m), 7.77 (1H, d, J=7.9 Hz), 8.03 (1H, d, J=5.7 Hz).

MS m/z 418 (M+H)⁺.

### Example 85

### sodium [(3S)-6-({(3S)-7-[propyl(pyridin-2-yl)amino]-2,3-dihydro-1-benzofuran-3-yl}amino)-2,3-dihydro-1-benzofuran-3-yl]acetate

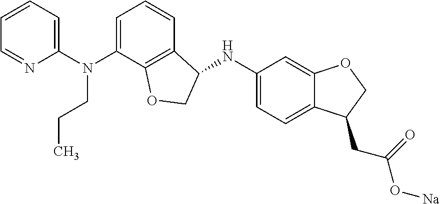

To a solution of methyl [(3S)-6-{[(3S)-7-(pyridin-2-ylamino)-2,3-dihydro-1-benzofuran-3-yl](trifluoroacetyl)amino}-2,3-dihydro-1-benzofuran-3-yl]acetate (0.310 g, 0.604 mmol) obtained in the process of obtaining Example 83 and n-propyl iodide (154 mg, 0.906 mmol) in N,N-dimethylformamide (4 mL) was added sodium hydride (60% in oil, 29.0 mg, 0.725 mmol) at 0° C., and the mixture was stirred at room temperature for 1.5 hr. The reaction mixture was poured into water, and the mixture was extracted with ethyl acetate. The extract was dried over anhydrous sodium sulfate, and concentrated under reduced pressure. The residue was purified by silica gel column chromatography (hexane:ethyl acetate=95:5-60:40) to give a mixture (0.143 g, a colorless oil) of methyl {(3S)-6-[{(3S)-7-[propyl(pyridin-2-yl)amino]-2,3-dihydro-1-benzofuran-3-yl}(trifluoroacetyl)amino]-2,3-dihydro-1-benzofuran-3-yl}acetate and methyl [(3S)-6-({(3S)-7-[propyl(pyridin-2-yl)amino]-2,3-dihydro-1-benzofuran-3-yl}amino)-2,3-dihydro-1-benzofuran-3-yl]acetate. To a mixed solution of the mixture (0.143 g) obtained above in tetrahydrofuran (2 mL) and methanol (4 mL) was added 1 M aqueous sodium hydroxide solution (0.934 mL), and the mixture was stirred at room temperature for 2 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure to give [(3S)-6-({(3S)-7-[propyl(pyridin-2-yl)amino]-2,3-dihydro-1-benzofuran-3-yl}amino)-2,3-dihydro-1-benzofuran-3-yl]acetic acid (0.140 g, yield 52%, 2 steps) as a pale-yellow oil. To a solution of [(3S)-6-({(3S)-7-[propyl(pyridin-2-yl)amino]-2,3-dihydro-1-benzofuran-3-yl}amino)-2,3-dihydro-1-benzofuran-3-yl]acetic acid (0.140 g, 0.314 mmol) obtained above in methanol (1 mL) was added 1 M aqueous sodium hydroxide solution (0.314 mL), and the mixture was stirred at room temperature for 1 min. Then, acetonitrile was added, and the mixture was concentrated under reduced pressure to give the title compound (132 mg, yield 90%) as a pale-yellow solid.

¹H NMR (300 MHz, DMSO-d₆) δ 0.85 (3H, t, J=7.3 Hz), 1.45-1.69 (2H, m), 1.99 (1H, dd, J=15.1, 10.2 Hz), 2.33 (1H, dd, J=15.3, 5.1 Hz), 3.47-3.63 (1H, m), 3.70-3.89 (2H, m), 4.07 (1H, dd, J=8.9, 7.3 Hz), 4.17 (1H, dd, J=9.4, 4.9 Hz), 4.61 (1H, t, J=8.9 Hz), 4.72 (1H, t, J=8.7 Hz), 5.19-5.35 (1H, m), 6.02 (1H, d, J=7.5 Hz), 6.06-6.27 (3H, m), 6.54-6.64 (1H, m), 6.86-7.03 (2H, m), 7.15 (1H, d, J=7.2 Hz), 7.22-7.43 (2H, m), 8.05-8.16 (1H, m).

MS m/z 446 (M+H)⁺ (as free form).

### Example 86

### sodium [(3S)-6-{[(3S)-7-(2,3-dihydro-4H-1,4-benzooxazin-4-yl)-2,3-dihydro-1-benzofuran-3-yl]amino}-2,3-dihydro-1-benzofuran-3-yl]acetate

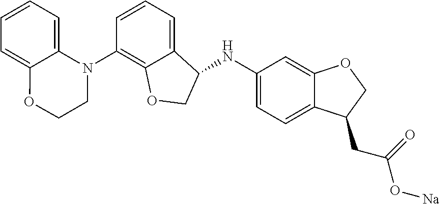

To a mixed solution of methyl [(3S)-6-{[(3S)-7-(2,3-dihydro-4H-1,4-benzooxazin-4-yl)-2,3-dihydro-1-benzofuran-3-yl](trifluoroacetyl)amino}-2,3-dihydro-1-benzofuran-3-yl]acetate (0.181 g, 0.327 mmol) in tetrahydrofuran (2 mL) and methanol (1 mL) was added 1 M aqueous sodium hydroxide solution (0.980 mL), and the mixture was stirred at room temperature for 2 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure to give [(3S)-6-{[(3S)-7-(2,3-dihydro-4H-1,4-benzooxazin-4-yl)-2,3-dihydro-1-benzofuran-3-yl]amino}-2,3-dihydro-1-benzofuran-3-yl]acetic acid (0.152 g, yield 100%) as a pale-yellow solid. To a solution of [(3S)-6-{[(3S)-7-(2,3-dihydro-4H-1,4-benzooxazin-4-yl)-2,3-dihydro-1-benzofuran-3-yl]amino}-2,3-dihydro-1-benzofuran-3-yl]acetic acid (0.152 g, 0.342 mmol) obtained above in methanol (1 mL) was added 1 M aqueous sodium hydroxide solution (0.342 mL), and the mixture was stirred at room temperature for 1 min. Then, acetonitrile was added and the mixture was concentrated under reduced pressure to give the title compound (97.3 mg, yield 61%) as a pale-yellow solid.

¹H NMR (300 MHz, DMSO-d₆) δ 1.98 (1H, dd, J=15.1, 10.2 Hz), 2.33 (1H, dd, J=15.1, 4.9 Hz), 3.46-3.69 (3H, m), 4.02-4.12 (1H, m), 4.15-4.31 (3H, m), 4.61 (1H, t, J=8.9 Hz), 4.70-4.81 (1H, m), 5.19-5.31 (1H, m), 5.98 (1H, d, J=7.9 Hz), 6.09-6.20 (2H, m), 6.45 (1H, dd, J=7.7, 2.1 Hz), 6.58-6.72 (2H, m), 6.74-6.81 (1H, m), 6.86-6.98 (2H, m), 7.08-7.23 (2H, m).

MS m/z 443 (M−H)⁻ (as free form).

### Example 87

### sodium [(3S)-6-{[(3S)-7-{[4-(methylsulfonyl)phenyl](propyl)amino}-2,3-dihydro-1-benzofuran-3-yl]amino}-2,3-dihydro-1-benzofuran-3-yl]acetate

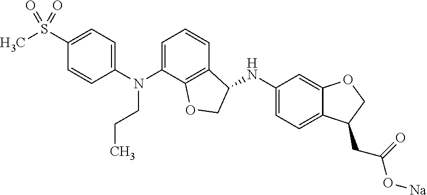

A solution of methyl [(3S)-6-{[(3S)-7-bromo-2,3-dihydro-1-benzofuran-3-yl](trifluoroacetyl)amino}-2,3-dihydro-1-benzofuran-3-yl]acetate (0.300 g, 0.600 mmol), 4-(methylsulfonyl)aniline (123 mg, 0.720 mmol) and cesium carbonate (0.585 g, 1.80 mmol) in toluene (4 mL) was substituted with argon, and tris(dibenzylideneacetone)dipalladium (0) (22.0 mg, 0.0240 mmol) and 4,5-bis(diphenylphosphino)-9,9-dimethylxanthene (27.8 mg, 0.0480 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-40:60) to give a mixture (0.161 g, a yellow solid) containing methyl [(3S)-6-{[(3S)-7-{[4-(methylsulfonyl)phenyl]amino}-2,3-dihydro-1-benzofuran-3-yl](trifluoroacetyl)amino}-2,3-dihydro-1-benzofuran-3-yl]acetate. To a solution of the solid (0.161 g) obtained above and n-propyl iodide (69.4 mg, 0.408 mmol) in N,N-dimethylformamide (3 mL) was added sodium hydride (60% in oil, 13.1 mg, 0.327 mmol) at 0° C., and the mixture was stirred at room temperature for 1.5 hr. The reaction mixture was poured into water, and the mixture was extracted with ethyl acetate. The extract was dried over anhydrous sodium sulfate, and concentrated under reduced pressure. The residue was purified by preparative HPLC to give a mixture (96.7 mg, a yellow oil) of methyl [(3S)-6-{[(3S)-7-{[4-(methylsulfonyl)phenyl](propyl)amino}-2,3-dihydro-1-benzofuran-3-yl](trifluoroacetyl)amino}-2,3-dihydro-1-benzofuran-3-yl]acetate and methyl [(3S)-6-{[(3S)-7-{[4-(methylsulfonyl)phenyl](propyl)amino}-2,3-dihydro-1-benzofuran-3-yl]amino}-2,3-dihydro-1-benzofuran-3-yl]acetate. To a mixed solution of the mixture (96.7 mg) obtained above in tetrahydrofuran (2 mL) and methanol (1 mL) was added 1 M aqueous sodium hydroxide solution (0.459 mL), and the mixture was stirred at room temperature for 2 hr. The reaction mixture was neutralized with 1 M hydrochloric acid, diluted with brine, and extracted with ethyl acetate. The extract was washed with saturated brine, dried over anhydrous sodium sulfate, and concentrated under reduced pressure to give a yellow oil (84.6 mg). To a solution of the oil (84.6 mg) obtained above in methanol (1 mL) was added 1 M aqueous sodium hydroxide solution (0.162 mL), and the mixture was stirred at room temperature for 1 min. Then, acetonitrile was added and the mixture was concentrated under reduced pressure to give the title compound (73.4 mg, yield 23% (4 steps)) as a pale-yellow solid.

¹H NMR (300 MHz, DMSO-d₆) δ 0.89 (3H, t, J=7.4 Hz), 1.50-1.70 (2H, m), 1.98 (1H, dd, J=15.0, 10.0 Hz), 2.32 (1H, dd, J=15.1, 4.9 Hz), 2.50 (3H, s), 3.44-3.72 (3H, m), 4.07 (1H, t, J=8.0 Hz), 4.18 (1H, dd, J=9.5, 4.9 Hz), 4.61 (1H, t, J=9.1 Hz), 4.72 (1H, t, J=8.7 Hz), 5.21-5.35 (1H, m), 6.01 (1H, d, J=7.6 Hz), 6.07-6.20 (2H, m), 6.69 (2H, d, J=9.1 Hz), 6.87-7.05 (2H, m), 7.16 (1H, d, J=7.6 Hz), 7.32 (1H, d, J=7.6 Hz), 7.60 (2H, d, J=8.7 Hz).

MS m/z 523 (M+H)⁺ (as free form).

### Example 88

### [(3S)-6-({(3S)-7-[(5-chloropyrimidin-2-yl)(propyl)amino]-2,3-dihydro-1-benzofuran-3-yl}amino)-2,3-dihydro-1-benzofuran-3-yl]acetic acid

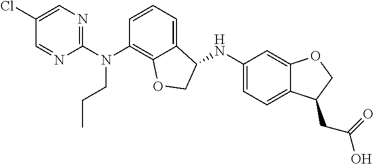

To a solution of methyl {(3S)-6-[{(3S)-7-[(5-chloropyrimidin-2-yl)amino]-2,3-dihydro-1-benzofuran-3-yl}(trifluoroacetyl)amino]-2,3-dihydro-1-benzofuran-3-yl}acetate (0.146 g, 0.266 mmol) and n-propyl iodide (62.0 mg, 0.399 mmol) in N,N-dimethylformamide (2 mL) was added sodium hydride (60% in oil, 12.8 mg, 0.319 mmol), and the mixture was stirred at room temperature for 20 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=95:5-70:30) to give a mixture (0.098 g, a solid) of methyl {(3S)-6-[{(3S)-7-[(5-chloropyrimidin-2-yl)(propyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(5-chloropyrimidin-2-yl)(propyl)amino]-2,3-dihydro-1-benzofuran-3-yl}amino)-2,3-dihydro-1-benzofuran-3-yl]acetate. To a mixed solution of the mixture (0.098 g) obtained above in tetrahydrofuran (5 mL) and methanol (5 mL) was added 2 M aqueous sodium hydroxide solution (3 mL), and the mixture was stirred at room temperature for 1 hr. The reaction mixture was neutralized with 1 M hydrochloric acid, and extracted with ethyl acetate. The extract was washed with saturated brine, dried over anhydrous magnesium sulfate, and concentrated under reduced pressure to give the title compound (0.078 g, yield 61%, 2 steps) as a solid.

¹H NMR (300 MHz, CDCl₃) δ 1.21-1.30 (3H, m), 1.59-1.69 (2H, m), 2.53-2.61 (1H, m), 2.72-2.80 (1H, m), 3.74-3.79 (1H, m), 3.80-3.87 (2H, m), 4.23-4.28 (1H, m), 4.35-4.40 (1H, m), 4.67-4.76 (2H, m), 5.20-5.23 (1H, m), 5.94 (2H, br s), 6.13-6.16 (2H, m), 6.94-6.99 (2H, m), 7.14 (1H, d, J=7.5 Hz), 7.29 (1H, d, J=7.2 Hz), 8.23 (2H, s).

### Example 89

### [(3S)-6-({(3S)-7-[(4-cyano-2-fluorophenyl)(ethyl)amino]-2,3-dihydro-1-benzofuran-3-yl}amino)-2,3-dihydro-1-benzofuran-3-yl]acetic acid

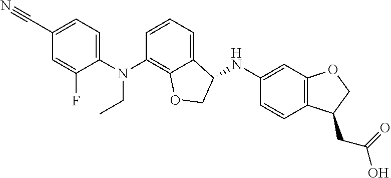

To a solution of methyl {(3S)-6-[{(3S)-7-[(4-cyano-2-fluorophenyl)amino]-2,3-dihydro-1-benzofuran-3-yl}(trifluoroacetyl)amino]-2,3-dihydro-1-benzofuran-3-yl}acetate (0.160 g, 0.288 mmol) and ethyl iodide (67.3 mg, 0.43 mmol) in N,N-dimethylformamide (2 mL) was added sodium hydride (60% in oil, 13.8 mg, 0.35 mmol), and the mixture was stirred at room temperature for 20 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=95:5-70:30) to give a mixture (0.085 g, an oil) of methyl {(3S)-6-[{(3S)-7-[(4-cyano-2-fluorophenyl)(ethyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(4-cyano-2-fluorophenyl)(ethyl)amino]-2,3-dihydro-1-benzofuran-3-yl}amino)-2,3-dihydro-1-benzofuran-3-yl]acetate. To a mixed solution of the mixture (0.085 g) obtained above in tetrahydrofuran (5 mL) and methanol (5 mL) was added 2 M aqueous sodium hydroxide solution (3 mL), and the mixture was stirred at room temperature for 1 hr. The reaction mixture was neutralized with 1 M hydrochloric acid, and extracted with ethyl acetate. The extract was washed with saturated brine, dried over anhydrous magnesium sulfate, and concentrated under reduced pressure to give the title compound (0.058 g, yield 43%, 2 steps) as a solid.

¹H NMR (300 MHz, CDCl₃) δ 1.22 (3H, t, J=7.2 Hz), 2.57-2.65 (1H, m), 2.76-2.83 (1H, m), 3.77-3.84 (3H, m), 4.24-4.30 (1H, m), 4.38-4.43 (1H, m), 4.62-4.77 (2H, m), 5.17-5.21 (1H, m), 6.02 (2H, br s), 6.16-6.20 (2H, m), 6.82-6.92 (2H, m), 6.97-7.01 (2H, m), 7.17-7.28 (3H, m).

### Example 90

### [(3S)-6-({(3S)-7-[(4-cyanophenyl)(ethyl)amino]-2,3-dihydro-1-benzofuran-3-yl}amino)-2,3-dihydro-1-benzofuran-3-yl]acetic acid

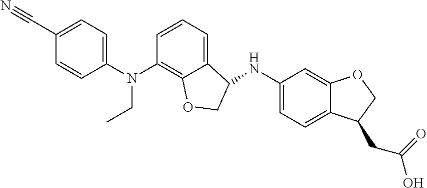

To a solution of methyl {(3S)-6-[{(3S)-7-[(4-cyanophenyl)amino]-2,3-dihydro-1-benzofuran-3-yl}(trifluoroacetyl)amino]-2,3-dihydro-1-benzofuran-3-yl}acetate (0.316 g, 0.59 mmol) and ethyl iodide (138 mg, 0.88 mmol) in N,N-dimethylformamide (2 mL) was added sodium hydride (60% in oil, 28 mg, 0.71 mmol), and the mixture was stirred at room temperature for 30 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=95:5-70:30) to give a mixture (0.140 g, a solid) of methyl {(3S)-6-[{(3S)-7-[(4-cyanophenyl)(ethyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(4-cyanophenyl)(ethyl)amino]-2,3-dihydro-1-benzofuran-3-yl}amino)-2,3-dihydro-1-benzofuran-3-yl]acetate. To a mixed solution of the mixture (0.140 g) obtained above in tetrahydrofuran (5 mL) and methanol (5 mL) was added 2 M aqueous sodium hydroxide solution (2 mL), and the mixture was stirred at room temperature for 1 hr. The reaction mixture was neutralized with 1 M hydrochloric acid, and extracted with ethyl acetate. The extract was washed with saturated brine, dried over anhydrous magnesium sulfate, and concentrated under reduced pressure to give the title compound (0.100 g, yield 37%, 2 steps) as a solid.

¹H NMR (300 MHz, CDCl₃) δ 1.23 (3H, t, J=6.9 Hz), 1.43 (1H, br s), 2.57-2.65 (1H, m), 2.77-2.84 (1H, m), 3.70-3.79 (3H, m), 4.25-4.30 (1H, m), 4.36-4.41 (1H, m), 4.69-4.77 (2H, m), 5.23-5.25 (1H, m), 6.14-6.17 (2H, m), 6.60-6.69 (2H, m), 6.94-7.01 (3H, m), 7.11 (1H, d, J=7.2 Hz), 7.31 (1H, d, J=7.2 Hz), 7.39 (2H, d, J=8.4 Hz).

### Example 91

### [(3S)-6-({(3S)-7-[(3-cyanophenyl)(ethyl)amino]-2,3-dihydro-1-benzofuran-3-yl}amino)-2,3-dihydro-1-benzofuran-3-yl]acetic acid

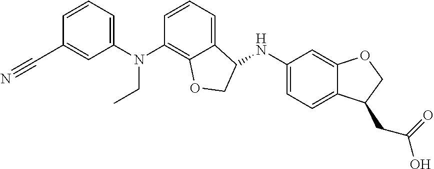

To a solution of methyl {(3S)-6-[{(3S)-7-[(3-cyanophenyl)amino]-2,3-dihydro-1-benzofuran-3-yl}(trifluoroacetyl)amino]-2,3-dihydro-1-benzofuran-3-yl}acetate (0.156 g, 0.29 mmol) and ethyl iodide (69 mg, 0.44 mmol) in N,N-dimethylformamide (2 mL) was added sodium hydride (60% in oil, 14 mg, 0.36 mmol), and the mixture was stirred at room temperature for 30 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=95:5-70:30) to give a mixture (0.057 g, an oil) of methyl {(3S)-6-[{(3S)-7-[(3-cyanophenyl)(ethyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(3-cyanophenyl)(ethyl)amino]-2,3-dihydro-1-benzofuran-3-yl}amino)-2,3-dihydro-1-benzofuran-3-yl]acetate. To a mixed solution of the mixture (0.057 g) obtained above in tetrahydrofuran (5 mL) and methanol (5 mL) was added 2 M aqueous sodium hydroxide solution (2 mL), and the mixture was stirred at room temperature for 1 hr. The reaction mixture was neutralized with 1 M hydrochloric acid to give a white solid as a precipitate. The obtained white solid was dried under reduced pressure to give the title compound (0.042 g, yield 32%, 2 steps) as a white solid.

¹H NMR (300 MHz, CDCl₃) δ 1.20 (3H, t, J=7.2 Hz), 2.50-2.66 (1H, m), 2.71-2.84 (1H, m), 3.65-3.80 (4H, m), 4.22-4.30 (1H, m), 4.45-4.47 (1H, m), 4.67-4.78 (2H, m), 5.23-5.27 (1H, m), 6.19-6.25 (2H, m), 6.85-6.86 (2H, m), 6.93-7.03 (3H, m), 7.10 (1H, d, J=7.5 Hz), 7.18-7.23 (2H, m), 7.33 (1H, d, J=7.2 Hz).

### Example 92

### [(3S)-6-({(3S)-7-[(3-cyanophenyl)(propyl)amino]-2,3-dihydro-1-benzofuran-3-yl}amino)-2,3-dihydro-1-benzofuran-3-yl]acetic acid

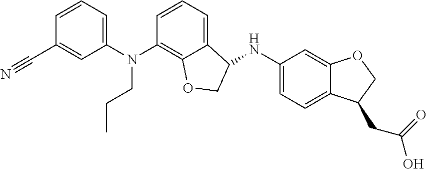

To a solution of methyl {(3S)-6-[{(3S)-7-[(3-cyanophenyl)amino]-2,3-dihydro-1-benzofuran-3-yl}(trifluoroacetyl)amino]-2,3-dihydro-1-benzofuran-3-yl}acetate (0.331 g, 0.616 mmol) and n-propyl iodide (126 mg, 0.74 mmol) in N,N-dimethylformamide (3 mL) was added sodium hydride (60% in oil, 37 mg, 0.924 mmol), and the mixture was stirred at room temperature for 30 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=95:5-70:30) to give a mixture (0.100 g, an oil) of methyl {(3S)-6-[{(3S)-7-[(3-cyanophenyl)(propyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(3-cyanophenyl)(propyl)amino]-2,3-dihydro-1-benzofuran-3-yl}amino)-2,3-dihydro-1-benzofuran-3-yl]acetate. To a mixed solution of the mixture (0.100 g) obtained above in tetrahydrofuran (10 mL) and methanol (10 mL) was added 1 M aqueous sodium hydroxide solution (6 mL), and the mixture was stirred at room temperature for 30 min. The reaction mixture was concentrated, and neutralized with 1 M hydrochloric acid to give a solid precipitate. The obtained solid was washed with water, and dried under reduced pressure to give the title compound (0.084 g, yield 29%, 2 steps) as a solid.

¹H NMR (300 MHz, CDCl₃) δ 0.94 (3H, t, J=7.8 Hz), 1.60-1.71 (2H, m), 2.57-2.66 (1H, m), 2.77-2.84 (1H, m), 3.55-3.60 (2H, m), 3.77-3.82 (1H, m), 4.25-4.30 (1H, m), 4.36-4.41 (1H, m), 4.69-4.78 (2H, m), 5.22-5.26 (1H, m), 6.15-6.18 (2H, m), 6.82-6.86 (2H, m), 6.94-7.02 (3H, m), 7.09-7.12 (1H, m), 7.17-7.30 (3H, m), 12.52 (1H, br s).

MS m/z 470 (M+H)⁺.

### Example 93

### [(3S)-6-({(3S)-7-[propyl(pyridin-3-yl)amino]-2,3-dihydro-1-benzofuran-3-yl}amino)-2,3-dihydro-1-benzofuran-3-yl]acetic acid

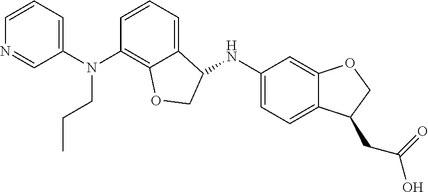

To a solution of methyl [(3S)-6-{[(3S)-7-(pyridin-3-ylamino)-2,3-dihydro-1-benzofuran-3-yl](trifluoroacetyl)amino}-2,3-dihydro-1-benzofuran-3-yl]acetate (0.289 g, 0.56 mmol) and n-propyl iodide (116 mg, 0.68 mmol) in N,N-dimethylformamide (2 mL) was added sodium hydride (60% in oil, 33.6 mg, 0.84 mmol), and the mixture was stirred at room temperature for 30 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=85:15-50:50) to give a mixture (0.105 g, an oil) of methyl {(3S)-6-[{(3S)-7-[propyl(pyridin-3-yl)amino]-2,3-dihydro-1-benzofuran-3-yl}(trifluoroacetyl)amino]-2,3-dihydro-1-benzofuran-3-yl}acetate and methyl [(3S)-6-({(3S)-7-[propyl(pyridin-3-yl)amino]-2,3-dihydro-1-benzofuran-3-yl}amino)-2,3-dihydro-1-benzofuran-3-yl]acetate. To a mixed solution of the mixture (0.105 g) obtained above in tetrahydrofuran (10 mL) and methanol (10 mL) was added 1 M aqueous sodium hydroxide solution (4 mL), and the mixture was stirred at room temperature for 1 hr. The reaction mixture was concentrated, and neutralized with 1 M hydrochloric acid to give a solid precipitate. The obtained solid was dried under reduced pressure to give the title compound (0.042 g, yield 17%, 2 steps) as a solid.

¹H NMR (300 MHz, CDCl₃) δ 0.94 (3H, t, J=7.5 Hz), 1.64-1.69 (2H, m), 2.48-2.60 (1H, m), 2.69-2.76 (1H, m), 3.62 (2H, t, J=7.5 Hz), 3.70-3.80 (1H, m), 4.17-4.24 (1H, m), 4.32-4.40 (1H, m), 4.67-4.72 (2H, m), 5.05 (2H, br s), 5.17-5.21 (1H, m), 6.10-6.13 (2H, m), 6.94-7.12 (5H, m), 7.24-7.26 (1H, m), 7.93-7.97 (2H, m).

MS m/z 446 (M+H)⁺.

### Example 94

### [(3S)-6-({(3S)-7-[(6-methylpyridin-3-yl)(propyl)amino]-2,3-dihydro-1-benzofuran-3-yl}amino)-2,3-dihydro-1-benzofuran-3-yl]acetic acid

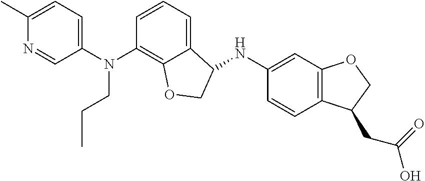

To a solution of methyl {(3S)-6-[{(3S)-7-[(6-methylpyridin-3-yl)amino]-2,3-dihydro-1-benzofuran-3-yl}(trifluoroacetyl)amino]-2,3-dihydro-1-benzofuran-3-yl}acetate (0.219 g, 0.42 mmol) and n-propyl iodide (85 mg, 0.5 mmol) in N,N-dimethylformamide (2 mL) was added sodium hydride (60% in oil, 25.2 mg, 0.63 mmol), and the mixture was stirred at room temperature for 30 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=85:15-50:50) to give a mixture (0.063 g, an oil) of methyl {(3S)-6-[{(3S)-7-[(6-methylpyridin-3-yl)(propyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(6-methylpyridin-3-yl)(propyl)amino]-2,3-dihydro-1-benzofuran-3-yl}amino)-2,3-dihydro-1-benzofuran-3-yl]acetate. To a mixed solution of the mixture (0.063 g) obtained above in tetrahydrofuran (10 mL) and methanol (10 mL) was added 1 M aqueous sodium hydroxide solution (4 mL), and the mixture was stirred at room temperature for 1 hr. The reaction mixture was concentrated, and neutralized with 1 M hydrochloric acid to give a solid precipitate. The obtained solid was dried under reduced pressure to give the title compound (0.024 g, yield 12%, 2 steps) as a solid.

¹H NMR (300 MHz, CDCl₃) δ 0.94 (3H, t, J=7.5 Hz), 1.61-1.71 (2H, m), 2.46 (3H, s), 2.50-2.55 (1H, m), 2.70-2.77 (1H, m), 3.62 (2H, t, J=7.5 Hz), 3.76-3.81 (1H, m), 4.18-4.23 (1H, m), 4.34-4.39 (1H, m), 4.64-4.73 (2H, m), 5.18-5.22 (1H, m), 5.34 (2H, br s), 6.12-6.14 (2H, m), 6.91-7.11 (4H, m), 7.23-7.26 (2H, m), 7.90-7.91 (1H, m).

MS m/z 460 (M+H)⁺.

### Example 95

### [(3S)-6-({(3S)-7-[(6-fluoropyridin-3-yl)(propyl)amino]-2,3-dihydro-1-benzofuran-3-yl}amino)-2,3-dihydro-1-benzofuran-3-yl]acetic acid

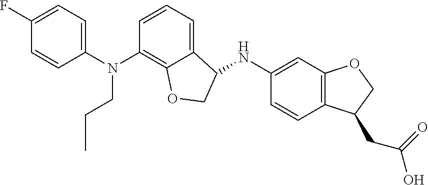

To a solution of methyl {(3S)-6-[{(3S)-7-[(6-fluoropyridin-3-yl)amino]-2,3-dihydro-1-benzofuran-3-yl}(trifluoroacetyl)amino]-2,3-dihydro-1-benzofuran-3-yl}acetate (0.263 g, 0.495 mmol) and n-propyl iodide (100 mg, 0.59 mmol) in N,N-dimethylformamide (2 mL) was added sodium hydride (60% in oil, 30 mg, 0.74 mmol), and the mixture was stirred at room temperature for 30 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=95:5-70:30) to give a mixture (0.103 g, an oil) of methyl {(3S)-6-[{(3S)-7-[(6-fluoropyridin-3-yl)(propyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(6-fluoropyridin-3-yl)(propyl)amino]-2,3-dihydro-1-benzofuran-3-yl}amino)-2,3-dihydro-1-benzofuran-3-yl]acetate. To a mixed solution of the mixture (0.103 g) obtained above in tetrahydrofuran (10 mL) and methanol (10 mL) was added 1 M aqueous sodium hydroxide solution (4 mL), and the mixture was stirred at room temperature for 1 hr. The reaction mixture was concentrated, and neutralized with 1 M hydrochloric acid to give a solid precipitate. The obtained solid was dried under reduced pressure to give the title compound (0.078 g, yield 32%, 2 steps) as a solid.

¹H NMR (300 MHz, CDCl₃) δ 0.94 (3H, t, J=7.5 Hz), 1.62-1.70 (2H, m), 2.57-2.65 (1H, m), 2.76-2.84 (1H, m), 3.59 (2H, t, J=7.5 Hz), 3.77-3.83 (1H, m), 4.24-4.30 (1H, m), 4.36-4.40 (1H, m), 4.66-4.77 (2H, m), 5.18-5.22 (1H, m), 6.14-6.17 (2H, m), 6.71-6.75 (1H, m), 6.91-7.01 (3H, m), 7.06-7.12 (2H, m), 7.22-7.25 (1H, m), 7.57 (1H, s), 12.48 (1H, br s).

MS m/z 464 (M+H)⁺.

### Example 96

### [(3S)-6-({(3S)-7-[(4-cyanophenyl)(propyl)amino]-2,3-dihydro-1-benzofuran-3-yl}amino)-2,3-dihydro-1-benzofuran-3-yl]acetic acid

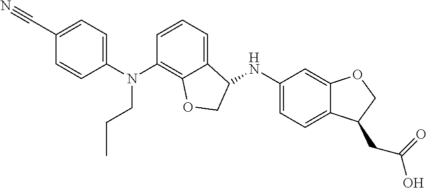

To a solution of methyl {(3S)-6-[{(3S)-7-[(4-cyanophenyl)amino]-2,3-dihydro-1-benzofuran-3-yl}(trifluoroacetyl)amino]-2,3-dihydro-1-benzofuran-3-yl}acetate (0.403 g, 0.75 mmol) and n-propyl iodide (153 mg, 0.9 mmol) in N,N-dimethylformamide (3 mL) was added sodium hydride (60% in oil, 45 mg, 1.125 mmol), and the mixture was stirred at room temperature for 30 min. The reaction mixture was poured into water, and the mixture was extracted with ethyl acetate. The extract was dried over anhydrous magnesium sulfate, and concentrated under reduced pressure. The residue was purified by silica gel column chromatography (hexane:ethyl acetate=95:5-70:30) to give a mixture (0.20 g, an oil) of methyl {(3S)-6-[{(3S)-7-[(4-cyanophenyl)(propyl)amino]-2,3-dihydro-1-benzofuran-3-yl}(trifluoroacetyl)amino]-2,3-dihydro-1-benzofuran-3-yl}acetate and methyl [(3S)-6-({(3S)-7-[(4-cyanophenyl)(propyl)amino]-2,3-dihydro-1-benzofuran-3-yl}amino)-2,3-dihydro-1-benzofuran-3-yl]acetate. To a mixed solution of the mixture (0.20 g) obtained above in tetrahydrofuran (5 mL) and methanol (5 mL) was added 1 M aqueous sodium hydroxide solution (6 mL), and the mixture was stirred at room temperature for 30 min. The reaction mixture was concentrated, and neutralized with 1 M hydrochloric acid to give a solid precipitate. The obtained solid was washed with water, and dried under reduced pressure to give the title compound (0.16 g, yield 45%, 2 steps) as a solid.

¹H NMR (300 MHz, CDCl₃) δ 0.93 (3H, t, J=7.2 Hz), 1.65-1.72 (2H, m), 2.54-2.65 (1H, m), 2.71-2.84 (1H, m), 3.60 (2H, t, J=7.8 Hz), 3.72-3.79 (2H, m), 4.22-4.30 (1H, m), 4.35-4.40 (1H, m), 4.69-4.78 (2H, m), 5.21-5.24 (1H, m), 6.13-6.15 (2H, m), 6.59 (2H, d, J=9.0 Hz), 6.95-7.01 (2H, m), 7.10 (1H, d, J=7.5 Hz), 7.31 (1H, d, J=7.2 Hz), 7.38 (2H, d, J=9.0), 12.49 (1H, br s).

### Example 97

### sodium [(3S)-6-({(3S)-7-[2-ethyl-4-(2,2,2-trifluoroethoxy)-1H-benzimidazol-1-yl]-2,3-dihydro-1-benzofuran-3-yl}amino)-2,3-dihydro-1-benzofuran-3-yl]acetate

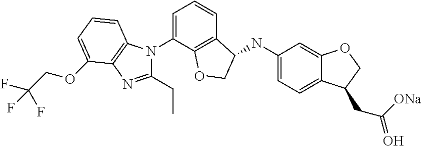

Methyl {(3S)-6-[{(3S)-7-[2-ethyl-4-(2,2,2-trifluoroethoxy)-1H-benzimidazol-1-yl]-2,3-dihydro-1-benzofuran-3-yl}(trifluoroacetyl)amino]-2,3-dihydro-1-benzofuran-3-yl}acetate (95 mg, 0.14 mmol) was dissolved in tetrahydrofuran (2 mL) and methanol (2 mL), 1N aqueous sodium hydroxide solution (1 mL) was added, and the mixture was stirred at 50° C. for 1 hr. The volatile component was evaporated under reduced pressure, and 1N aqueous hydrochloric acid solution (1 mL) was added. The mixture was diluted with saturated brine, and extracted with ethyl acetate. The extract was dried over anhydrous magnesium sulfate and the volatile component was evaporated under reduced pressure to give a white solid (76 mg, yield—100%). The obtained solid (55 mg, 0.1 mmol) was dissolved in tetrahydrofuran (2 mL), and 1 M aqueous sodium hydroxide solution (0.1 mL) was added. The volatile component was evaporated under reduced pressure, and the solid was precipitated to give the title compound (50 mg) as a white solid.

¹H NMR (300 MHz, DMSO-d₆) δ 1.18-1.28 (3H, m), 1.95 (1H, dd, J=14.9, 10.0 Hz), 2.29 (1H, dd, J=14.9, 5.1 Hz), 2.65-2.77 (2H, m), 3.53 (1H, br s), 4.03-4.11 (1H, m), 4.19-4.28 (1H, m), 4.60 (1H, t, J=8.9 Hz), 4.79 (1H, dd, J=8.5, 3.2 Hz), 5.13 (2H, q, J=9.0 Hz), 5.37 (1H, d, J=7.5 Hz), 6.05-6.21 (3H, m), 6.73 (1H, dd, J=19.4, 7.7 Hz), 6.90 (2H, dd, J=15.6, 8.1 Hz), 7.06-7.17 (2H, m), 7.39 (1H, d, J=7.5 Hz), 7.53 (1H, d, J=7.5 Hz).

MS m/z 554 (M+H)⁺ (as free form).

### Example 98

### sodium [(3S)-6-{[(3S)-7-{2-ethyl-4-[3-(methylsulfonyl)propoxy]-1H-benzimidazol-1-yl}-2,3-dihydro-1-benzofuran-3-yl]amino}-2,3-dihydro-1-benzofuran-3-yl]acetate

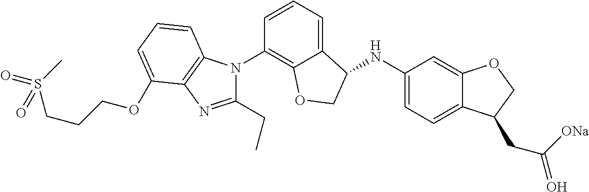

Methyl [(3S)-6-{[(3S)-7-{2-ethyl-4-[3-(methylsulfonyl)propoxy]-1H-benzimidazol-1-yl}-2,3-dihydro-1-benzofuran-3-yl](trifluoroacetyl)amino}-2,3-dihydro-1-benzofuran-3-yl]acetate (110 mg, 0.16 mmol) was dissolved in tetrahydrofuran (2 mL) and methanol (2 mL), 1N aqueous sodium hydroxide solution (1 mL) was added, and the mixture was stirred at 50° C. for 1 hr. The volatile component was evaporated under reduced pressure, and 1 M aqueous hydrochloric acid solution (1 mL) was added. The mixture was diluted with saturated brine, and extracted with ethyl acetate. The extract was dried over anhydrous magnesium sulfate and the volatile component was evaporated under reduced pressure to give a white solid (96 mg, yield—100%). The obtained solid (59 mg, 0.1 mmol) was dissolved in tetrahydrofuran (2 mL), and 1 M aqueous sodium hydroxide solution (0.1 mL) was added. The volatile component was evaporated under reduced pressure, and the solid was precipitated to give the title compound (46 mg) as a white solid.

¹H NMR (300 MHz, DMSO-d₆) δ 1.15-1.29 (m, 3H), 2.01-2.14 (m, 1H), 2.17-2.29 (2H, m), 2.35-2.45 (1H, m), 2.62-2.75 (2H, m), 3.05 (3H, s), 3.30-3.40 (3H, m), 3.50-3.62 (2H, m), 4.07 (1H, t, J=7.9 Hz), 4.18-4.27 (1H, m), 4.60 (1H, t, J=8.9 Hz), 4.72-4.85 (1H, m), 5.32-5.42 (1H, m), 6.06-6.22 (3H, m), 6.64 (1H, dd, J=20.0, 7.9 Hz), 6.76 (1H, d, J=8.3 Hz), 6.93 (1H, d, J=7.5 Hz), 7.02-7.17 (2H, m), 7.37 (1H, d, J=7.9 Hz), 7.52 (1H, d, J=7.5 Hz).

MS m/z 592 (M+H)⁺ (as free form).

### Example 99

### sodium [(3S)-6-({(3S)-7-[2-methyl-6-(2,2,2-trifluoroethoxy)-1H-benzimidazol-1-yl]-2,3-dihydro-1-benzofuran-3-yl}amino)-2,3-dihydro-1-benzofuran-3-yl]acetate

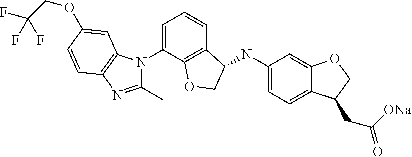

Methyl {(3S)-6-[{(3S)-7-[2-methyl-6-(2,2,2-trifluoroethoxy)-1H-benzimidazol-1-yl]-2,3-dihydro-1-benzofuran-3-yl}(trifluoroacetyl)amino]-2,3-dihydro-1-benzofuran-3-yl}acetate (150 mg, 0.24 mmol) was dissolved in tetrahydrofuran (2 mL) and methanol (2 mL), 1 M aqueous sodium hydroxide solution (1 mL) was added, and the mixture was stirred at 50° C. for 2 hr. The volatile component was evaporated under reduced pressure, and 1 M aqueous hydrochloric acid solution (1 mL) was added to allow precipitation of a white solid. The resulting solid was collected (110 mg, 0.17 mmol). The solid (74 mg, 0.12 mmol) was dissolved in tetrahydrofuran (2 mL), and 1 M aqueous sodium hydroxide solution (0.12 mL) was added. The volatile component was evaporated under reduced pressure and the solid was precipitated, whereby the title compound (75 mg) was obtained as a white solid.

¹H NMR (300 MHz, DMSO-d₆) δ 2.04-2.15 (1H, m), 2.34-2.45 (4H, m), 3.53-3.58 (1H, m), 4.04-4.11 (1H, m), 4.26 (1H, dd, J=9.5, 4.5 Hz), 4.56-4.87 (4H, m), 5.31-5.43 (1H, m), 6.05-6.21 (3H, m), 6.71 (1H, t, J=2.8 Hz), 6.90-6.99 (2H, m), 7.09-7.18 (1H, m), 7.41 (1H, d, J=8.0 Hz), 7.50-7.57 (2H, m).

MS m/z 540 (M+H)⁺ (as free form).

### Example 100

### methyl [(3S)-6-{[(2-chloro-2′,6′-dimethylbiphenyl-3-yl)methyl]amino}-2,3-dihydro-1-benzofuran-3-yl]acetate

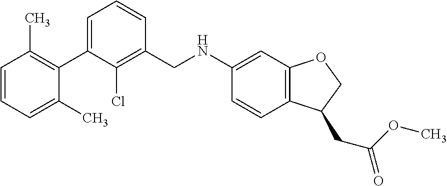

A solution of 2-chloro-2′,6′-dimethylbiphenyl-3-carbaldehyde (0.184 g, 0.750 mmol), methyl. [(3S)-6-amino-2,3-dihydro-1-benzofuran-3-yl]acetate (0.155 g, 0.750 mmol), and acetic acid (0.086 mL, 1.50 mmol) in acetonitrile (4 mL) was stirred under a nitrogen atmosphere at room temperature for 2 hr. Sodium triacetoxyborohydride (0.397 g, 1.50 mmol) was added to the reaction mixture, and the mixture was further stirred for 3 hr. The reaction mixture was treated with water and saturated aqueous ammonium chloride solution, and extracted with ethyl acetate. The extract was washed successively with saturated aqueous sodium hydrogen carbonate and saturated brine, dried over anhydrous sodium sulfate, and concentrated under reduced pressure. The residue was purified by silica gel column chromatography (ethyl acetate:hexane=5:95-30:70) and preparative HPLC to give the title compound (0.294 g, yield 90%) as a colorless oil.

¹H NMR (300 MHz, CDCl₃) δ 1.99 (6H, s), 2.49-2.59 (1H, m), 2.69-2.78 (1H, m), 3.69-3.82 (4H, m), 4.18-4.29 (2H, m), 4.47 (2H, s), 4.71 (1H, t, J=8.9 Hz), 6.14 (1H, d, J=2.1 Hz), 6.18 (1H, dd, J=7.9, 2.1 Hz), 6.94 (1H, d, J=7.9 Hz), 7.06 (1H, dd, J=7.5, 1.7 Hz), 7.10-7.16 (2H, m), 7.18-7.25 (1H, m), 7.25-7.31 (1H, m), 7.41 (1H, dd, J=7.7, 1.7 Hz).

MS m/z 436 (M+H)⁺.

### Example 101

### [(3S)-6-{[(2-chloro-2′,6′-dimethylbiphenyl-3-yl)methyl]amino}-2,3-dihydro-1-benzofuran-3-yl]acetic acid

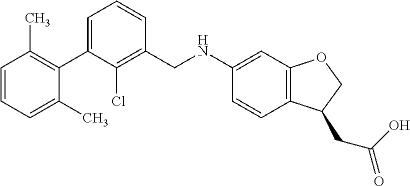

To a mixed solution of methyl [(3S)-6-{[(2-chloro-2′,6′-dimethylbiphenyl-3-yl)methyl]amino}-2,3-dihydro-1-benzofuran-3-yl]acetate (0.290 g, 0.665 mmol) in methanol (1.3 mL) and tetrahydrofuran (2.6 mL) was added 1 M aqueous sodium hydroxide solution (1.3 mL), and the mixture was stirred at 50° C. for 2 hr. The reaction mixture was neutralized with 1 M hydrochloric acid, and extracted with ethyl acetate. The extract was washed with saturated brine, dried over anhydrous sodium sulfate, and concentrated under reduced pressure. The residue was purified by silica gel column chromatography (ethyl acetate:hexane=20:80-80:20), and the obtained oil was dissolved in a mixed solution of methanol (1.3 mL) and tetrahydrofuran (2.6 mL). The mixture was treated with 1 M aqueous sodium hydroxide solution (1.3 mL), and concentrated under reduced pressure. The residue was dissolved in water, and neutralized with 1 N hydrochloric acid. The precipitated solid was collected by filtration, and dried to give the title compound (0.233 g, yield 83%) as a colorless powder.

¹H NMR (300 MHz, CDCl₃) δ 1.99 (6H, s), 2.54-2.66 (1H, m), 2.74-2.85 (1H, m), 3.72-3.84 (1H, m), 4.25 (1H, dd, J=9.2, 6.0 Hz), 4.47 (2H, s), 4.72 (1H, t, J=9.0 Hz), 6.14 (1H, d, J=2.1 Hz), 6.19 (1H, dd, J=8.0, 2.1 Hz), 6.97 (1H, d, J=8.0 Hz), 7.06 (1H, dd, J=7.3, 1.7 Hz), 7.10-7.16 (2H, m), 7.18-7.25 (1H, m), 7.25-7.32 (1H, m), 7.41 (1H, dd, J=7.5, 1.7 Hz).

MS m/z 422 (M+H)⁺.

### Example 102

340

### methyl [(3S)-6-{[(2-hydroxy-2′,6′-dimethylbiphenyl-3-yl)methyl]amino}-2,3-dihydro-1-benzofuran-3-yl]acetate

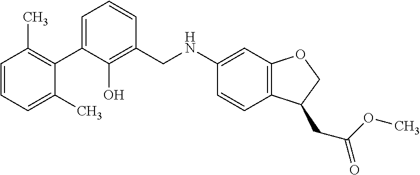

A solution of 2-hydroxy-2′,6′-dimethylbiphenyl-3-carbaldehyde (0.453 g, 2.00 mmol), methyl [(3S)-6-amino-2,3-dihydro-1-benzofuran-3-yl]acetate (0.414 g, 2.00 mmol), and acetic acid (0.343 mL, 6.00 mmol) in acetonitrile (10 mL) was stirred under a nitrogen atmosphere at room temperature for 2 hr. Sodium triacetoxyborohydride (0.397 g, 1.50 mmol) was added to the reaction mixture, and the mixture was further stirred for 3 hr. The reaction mixture was treated with water and saturated aqueous ammonium chloride solution, and the mixture was extracted with ethyl acetate. The extract was washed successively with saturated aqueous sodium hydrogen carbonate and saturated brine, dried over anhydrous sodium sulfate, and concentrated under reduced pressure. The residue was purified by silica gel column chromatography (ethyl acetate:hexane=10:90-50:50) to give the title compound (0.714 g, yield 86%) as a yellow oil.

¹H NMR (300 MHz, CDCl₃) δ 2.04 (6H, s), 2.48-2.59 (1H, m), 2.68-2.78 (1H, m), 3.71 (4H, s), 4.09 (1H, s), 4.22 (1H, dd, J=9.1, 6.1 Hz), 4.40 (2H, s), 4.71 (1H, t, J=9.1 Hz), 6.24-6.30 (2H, m), 6.58 (1H, br s), 6.90-7.00 (3H, m), 7.11-7.25 (4H, m).

MS m/z 418 (M+H)⁺.

### Example 103

### methyl [(3S)-6-{[(2-methoxy-2′,6′-dimethylbiphenyl-3-yl)methyl]amino}-2,3-dihydro-1-benzofuran-3-yl]acetate

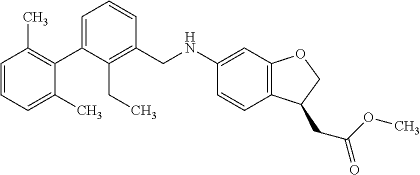

A solution of methyl [(3S)-6-{[(2-hydroxy-2′,6′-dimethylbiphenyl-3-yl)methyl]amino}-2,3-dihydro-1-benzofuran-3-yl]acetate (0.265 g, 0.635 mmol), methanol (0.031 mL, 0.762 mmol) and tributylphosphine (0.253 mL, 1.02 mmol) in tetrahydrofuran (5 mL) was stirred, 1,1′-(azodicarbonyl)dipiperidine (0.256 g, 1.02 mmol) was added, and the mixture was stirred under a nitrogen atmosphere at room temperature for 3 hr. Hexane (5 mL) was added to the reaction mixture, the precipitated insoluble material was filtered off, and the filtrate was concentrated under reduced pressure. The residue was purified by silica gel column chromatography (ethyl acetate:hexane=5:95-30:70) to give the title compound (0.252 g, yield 92%) as a colorless oil.

¹H NMR (300 MHz, CDCl₃) δ 2.08 (6H, s), 2.48-2.59 (1H, m), 2.69-2.78 (1H, m), 3.34 (3H, s), 3.69-3.82 (4H, m), 4.15 (1H, br s), 4.22 (1H, dd, J=9.1, 6.1 Hz), 4.36 (2H, s), 4.71 (1H, t, J=9.1 Hz), 6.16-6.23 (2H, m), 6.93 (1H, d, J=7.6 Hz), 6.98 (1H, dd, J=7.6, 1.9 Hz), 7.07-7.14 (3H, m), 7.15-7.21 (1H, m), 7.33 (1H, dd, J=7.4, 1.7 Hz).

MS m/z 432 (M+H)⁺.

### Example 104

### [(3S)-6-{[(2-methoxy-2′,6′-dimethylbiphenyl-3-yl)methyl]amino}-2,3-dihydro-1-benzofuran-3-yl]acetic acid

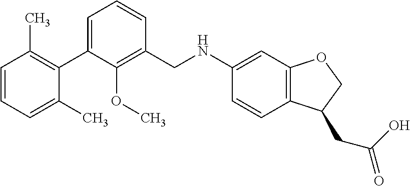

To a mixed solution of methyl [(3S)-6-{[(2-methoxy-2′,6′-dimethylbiphenyl-3-yl)methyl]amino}-2,3-dihydro-1-benzofuran-3-yl]acetate (0.248 g, 0.575 mmol) in methanol (1.2 mL) and tetrahydrofuran (2.4 mL) was added 1 M aqueous sodium hydroxide solution (1.2 mL), and the mixture was stirred at 50° C. for 2 hr. The reaction mixture was concentrated under reduced pressure, and the residue was dissolved in water. The solution was neutralized with 1 M hydrochloric acid, and the precipitated solid was collected by filtration, and dried to give the title compound (0.217 g, yield 90%) as a colorless powder.

¹H NMR (300 MHz, CDCl₃) δ 2.08 (6H, s), 2.53-2.65 (1H, m), 2.74-2.84 (1H, m), 3.34 (3H, s), 3.71-3.83 (1H, m), 4.24 (1H, dd, J=9.1, 6.0 Hz), 4.36 (2H, s), 4.72 (1H, t, J=9.1 Hz), 6.17-6.25 (2H, m), 6.93-7.01 (2H, m), 7.07-7.14 (3H, m), 7.15-7.22 (1H, m), 7.33 (1H, dd, J=7.5, 1.9 Hz).

MS m/z 418 (M+H)⁺.

### Example 105

### methyl [(3S)-6-({[2′,6′-dimethyl-2-(1-methylethoxy)biphenyl-3-yl]methyl}amino)-2,3-dihydro-1-benzofuran-3-yl]acetate

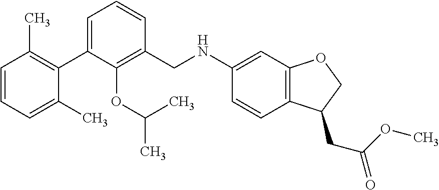

In the same manner as in Example 103, the title compound was obtained as a colorless oil from methyl [(3S)-6-{[(2-hydroxy-2′,6′-dimethylbiphenyl-3-yl)methyl]amino}-2,3-dihydro-1-benzofuran-3-yl]acetate and 2-propanol. yield 88%.

¹H NMR (300 MHz, CDCl₃) δ 0.93 (6H, d, J=6.1 Hz), 2.10 (6H, s), 2.48-2.58 (1H, m), 2.69-2.78 (1H, m), 3.56-3.66 (1H, m), 3.69-3.82 (4H, m), 4.17-4.26 (2H, m), 4.37 (2H, s), 4.71 (1H, t, J=8.9 Hz), 6.15-6.22 (2H, m), 6.92 (1H, d, J=7.6 Hz), 6.96-7.01 (1H, m), 7.04-7.13 (3H, m), 7.13-7.20 (1H, m), 7.32 (1H, dd, J=7.4, 1.7 Hz).

MS m/z 460 (M+H)⁺.

### Example 106

### [(3S)-6-({[2′,6′-dimethyl-2-(1-methylethoxy)biphenyl-3-yl]methyl}amino)-2,3-dihydro-1-benzofuran-3-yl]acetic acid

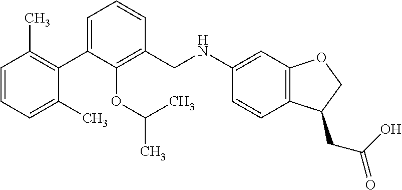

In the same manner as in Example 104, the title compound was obtained as a colorless powder from methyl [(3S)-6-({[2′,6′-dimethyl-2-(1-methylethoxy)biphenyl-3-yl]methyl}amino)-2,3-dihydro-1-benzofuran-3-yl]acetate. yield 88%.

¹H NMR (300 MHz, CDCl₃) δ 0.93 (6H, d, J=6.0 Hz), 2.10 (6H, s), 2.54-2.65 (1H, m), 2.75-2.84 (1H, m), 3.54-3.68 (1H, m), 3.71-3.82 (1H, m), 4.24 (1H, dd, J=9.0, 6.0 Hz), 4.38 (2H, s), 4.72 (1H, t, J=9.0 Hz), 6.16-6.23 (2H, m), 6.93-7.01 (2H, m), 7.04-7.13 (3H, m), 7.13-7.20 (1H, m), 7.32 (1H, dd, J=7.5, 1.7 Hz).

MS m/z 446 (M+H)⁺.

### Example 107

### [(3S)-6-{[(2-hydroxy-2′,6′-dimethylbiphenyl-3-yl)methyl]amino}-2,3-dihydro-1-benzofuran-3-yl]acetic acid

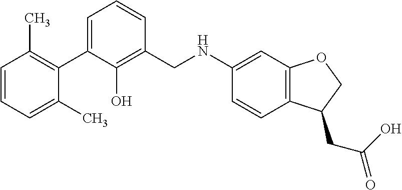

In the same manner as in Example 101, the title compound was obtained as a beige powder from methyl [(3S)-6-{[(2-hydroxy-2′,6′-dimethylbiphenyl-3-yl)methyl]amino}-2,3-dihydro-1-benzofuran-3-yl]acetate. yield 46%.

¹H NMR (300 MHz, CDCl₃) δ 2.04 (6H, s), 2.53-2.65 (1H, m), 2.73-2.84 (1H, m), 3.72-3.83 (1H, m), 4.25 (1H, dd, J=9.1, 6.0 Hz), 4.40 (2H, s), 4.72 (1H, t, J=9.1 Hz), 6.24-6.32 (2H, m), 6.90-7.01 (3H, m), 7.10-7.25 (4H, m).

MS m/z 404 (M+H)⁺.

### Example 108

### methyl [(3S)-6-({3-[1-(difluoromethyl)-3,5-dimethyl-1H-pyrazol-4-yl]-2-methylbenzyl}amino)-2,3-dihydro-1-benzofuran-3-yl]acetate

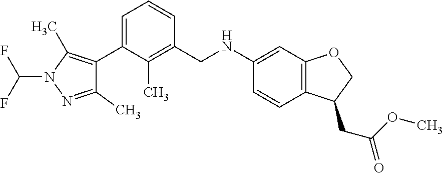

To a solution of {3-[1-(difluoromethyl)-3,5-dimethyl-1H-pyrazol-4-yl]-2-methylphenyl}methanol (0.133 g, 0.500 mmol), methyl [(3S)-6-{[(2-nitrophenyl)sulfonyl]amino}-2,3-dihydro-1-benzofuran-3-yl]acetate (0.196 g, 0.500 mmol) and triphenylphosphine (0.210 g, 0.800 mmol) in tetrahydrofuran (4 mL) was added 40% diethyl azodicarboxylate toluene solution (0.317 mL, 0.800 mmol) at room temperature, and the mixture was stirred for 5 hr under a nitrogen atmosphere. The reaction mixture was concentrated under reduced pressure, and the residue was purified by silica gel column chromatography (ethyl acetate:hexane=20:80-60:40) to give a yellow foam. To a solution of this product and mercaptoacetic acid (0.070 mL, 1.00 mmol) in N,N-dimethylformamide (1 mL) was added lithium hydroxide monohydrate (0.084 g, 2.00 mmol), and the mixture was stirred at room temperature for 3 hr. Ethyl acetate was added to the reaction mixture, and the mixture was washed successively with saturated aqueous sodium hydrogen carbonate and saturated brine, dried over anhydrous sodium sulfate, and concentrated under reduced pressure. The residue was purified by silica gel column chromatography (ethyl acetate:hexane=5:95-40:60) to give the title compound (0.155 g, yield 68%, 2 steps) as a colorless oil.

¹H NMR (300 MHz, CDCl₃) δ 2.07 (3H, s), 2.11 (3H, s), 2.24 (3H, s), 2.49-2.60 (1H, m), 2.70-2.79 (1H, m), 3.71 (3H, s), 3.73-3.83 (1H, m), 3.94 (1H, br s), 4.23 (1H, dd, J=9.0, 6.0 Hz), 4.29 (2H, s), 4.72 (1H, t, J=9.0 Hz), 6.14 (1H, d, J=2.2 Hz), 6.19 (1H, dd, J=8.0, 2.2 Hz), 6.96 (1H, d, J=7.5 Hz), 6.98-7.39 (4H, m).

MS m/z 456 (M+H)⁺.

### Example 109

### [(3S)-6-({3-[1-(difluoromethyl)-3,5-dimethyl-1H-pyrazol-4-yl]-2-methylbenzyl}amino)-2,3-dihydro-1-benzofuran-3-yl]acetic acid

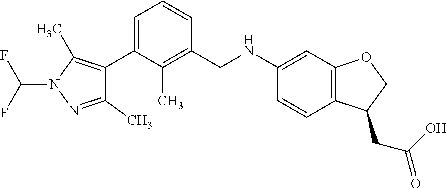

In the same manner as in Example 104, the title compound was obtained as a colorless powder from methyl [(3S)-6-({3-[1-(difluoromethyl)-3,5-dimethyl-1H-pyrazol-4-yl]-2-methylbenzyl}amino)-2,3-dihydro-1-benzofuran-3-yl]acetate. yield 75%.

¹H NMR (300 MHz, CDCl₃) δ 2.07 (3H, s), 2.11 (3H, s), 2.24 (3H, s), 2.55-2.67 (1H, m), 2.75-2.86 (1H, m), 3.73-3.85 (1H, m), 4.22-4.33 (3H, m), 4.74 (1H, t, J=9.0 Hz), 6.15 (1H, d, J=2.1 Hz), 6.20 (1H, dd, J=8.1, 2.1 Hz), 6.99 (1H, d, J=8.1 Hz), 7.01-7.44 (4H, m).

MS m/z 442 (M+H)⁺.

### Example 110

### methyl {(3S)-6-[(3-bromo-2-methylbenzyl)amino]-2,3-dihydro-1-benzofuran-3-yl}acetate

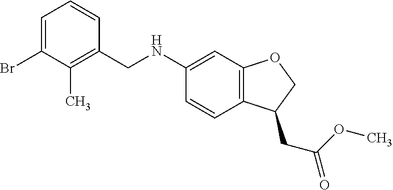

Methyl 3-bromo-2-methylbenzoate (2.29 g, 10.0 mmol) was dissolved in tetrahydrofuran (50 mL), under ice-cooling, lithium aluminum hydride (0.285 g, 7.50 mmol) was added by small portions, and the mixture was stirred under a nitrogen atmosphere for 2 hr. Sodium sulfate 10 hydrate (2.42 g, 7.50 mmol) was added to the reaction mixture, and the mixture was stirred at room temperature for 2 hr. Insoluble material was filtered off through celite, and the filtrate was concentrated under reduced pressure. The obtained solid was recrystallized from heptane-ethyl acetate to give 3-bromo-2-methylbenzyl alcohol (1.76 g, yield 88%) as colorless crystals. This product (0.943 g, 4.69 mmol) was dissolved in acetonitrile (25 mL), a Dess-Martin reagent (2.39 g, 5.63 mmol) was added by small portions under ice-cooling, and the mixture was stirred at room temperature for 0.5 hr. The reaction mixture was treated with saturated aqueous sodium hydrogen carbonate and aqueous sodium thiosulfate solution, and the mixture was extracted with ethyl acetate. The extract was washed with saturated brine, dried over anhydrous magnesium sulfate, and concentrated under reduced pressure. A solution of the obtained colorless oil, methyl [(3S)-6-amino-2,3-dihydro-1-benzofuran-3-yl]acetate (0.972 g, 4.69 mmol) and acetic acid (0.537 mL, 9.38 mmol) in acetonitrile (25 mL) was stirred under a nitrogen atmosphere at room temperature for 0.5 hr. Sodium triacetoxyborohydride (1.99 g, 9.38 mmol) was added to the reaction mixture, and the mixture was further stirred for 12 hr. The reaction mixture was treated with water and saturated aqueous ammonium chloride solution, and extracted with ethyl acetate. The extract was washed successively with saturated aqueous sodium hydrogen carbonate and saturated brine, dried over anhydrous sodium sulfate, and concentrated under reduced pressure. The residue was purified by silica gel column chromatography (ethyl acetate:hexane=5:95-40:60) to give the title compound (1.59 g, yield 87%, 2 steps) as a colorless oil.

¹H NMR (300 MHz, CDCl₃) δ 2.42 (3H, s), 2.48-2.59 (1H, m), 2.68-2.78 (1H, m), 3.68-3.83 (4H, m), 3.89 (1H, br s), 4.18-4.30 (3H, m), 4.71 (1H, t, J=9.1 Hz), 6.07-6.16 (2H, m), 6.93 (1H, d, J=8.0 Hz), 7.01 (1H, t, J=7.9 Hz), 7.23-7.30 (1H, m), 7.49 (1H, dd, J=7.9, 1.1 Hz).

MS m/z 390 (M+H)⁺.

### Example 111

### methyl [(3S)-6-{[2-methyl-3-(6-methyl-2,3-dihydro-4H-pyrido[3,2-b] [1,4]oxazin-4-yl)benzyl]amino}-2,3-dihydro-1-benzofuran-3-yl]acetate

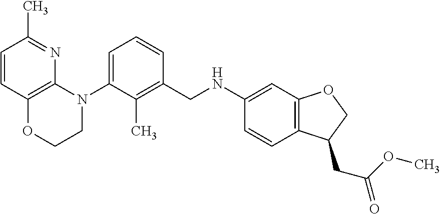

To a mixture of methyl {(3S)-6-[(3-bromo-2-methylbenzyl)amino]-2,3-dihydro-1-benzofuran-3-yl}acetate (0.312 g, 0.800 mmol), 6-methyl-3,4-dihydro-2H-pyrido[3,2-b] [1,4]oxazine (0.180 g, 1.20 mmol) and cesium carbonate (0.521 g, 1.60 mmol) in toluene (8 mL) were added tris(dibenzylideneacetone)dipalladium (0) (0.037 g, 0.040 mmol) and 4,5-bis(diphenylphosphino)-9,9-dimethylxanthene (0.046 g, 0.080 mmol), and the mixture was stirred under an argon atmosphere at 100° C. for 21 hr. To the reaction mixture were added water and ethyl acetate, and the insoluble material was filtered off through celite. The organic layer of the filtrate was washed with saturated brine, dried over anhydrous magnesium sulfate, and concentrated under reduced pressure. The residue was purified by silica gel column chromatography (ethyl acetate:hexane=10:90-50:50) to give the title compound (0.310 g, yield 84%) as a yellow foam.

¹H NMR (300 MHz, CDCl₃) δ 2.17 (3H, s), 2.19 (3H, s), 2.48-2.60 (1H, m), 2.69-2.79 (1H, m), 3.60-3.89 (6H, m), 3.95 (1H, br s), 4.19-4.36 (5H, m), 4.72 (1H, t, J=8.9 Hz), 6.12-6.20 (2H, m), 6.40 (1H, d, J=8.1 Hz), 6.90-6.97 (2H, m), 7.12-7.29 (3H, m).

MS m/z 460 (M+H)⁺.

### Example 112

### [(3S)-6-{[2-methyl-3-(6-methyl-2,3-dihydro-4H-pyrido[3,2-b′] [1,4]oxazin-4-yl)benzyl]amino}-2,3-dihydro-1-benzofuran-3-yl]acetic acid

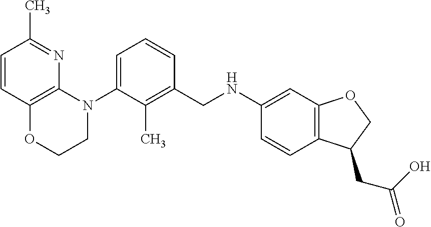

In the same manner as in Example 104, the title compound was obtained as a beige powder from methyl [(3S)-6-{[2-methyl-3-(6-methyl-2,3-dihydro-4H-pyrido[3,2-b] [1,4]oxazin-4-yl)benzyl]amino}-2,3-dihydro-1-benzofuran-3-yl]acetate. yield 84%.

¹H NMR (300 MHz, CDCl₃) δ 2.15 (3H, d, J=4.2 Hz), 2.21 (3H, s), 2.31-2.48 (1H, m), 2.58-2.70 (1H, m), 3.60-3.88 (3H, m), 4.13-4.36 (5H, m), 4.63-4.75 (1H, m), 6.12-6.19 (2H, m), 6.41 (1H, d, J=7.6 Hz), 6.90 (1H, dd, J=8.5, 2.8 Hz), 6.96 (1H, d, J=8.0 Hz), 7.11-7.31 (3H, m).

MS m/z 446 (M+H)⁺.

### Example 113

### methyl [(3S)-6-({3-[2-(difluoromethoxy)-4,6-dimethylpyrimidin-5-yl]-2-methylbenzyl}amino)-2,3-dihydro-1-benzofuran-3-yl]acetate

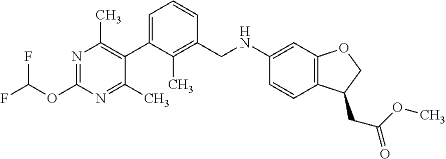

In the same manner as in Reference Example 84, the title compound was obtained as a colorless oil from methyl [(3S)-6-{[2-methyl-3-(4,4,5,5-tetramethyl-1,3,2-dioxaborolan-2-yl)benzyl]amino}-2,3-dihydro-1-benzofuran-3-yl]acetate and 5-bromo-2-(difluoromethoxy)-4,6-dimethylpyrimidine. yield 15% (2 steps).

¹H NMR (300 MHz, CDCl₃) δ 2.01 (3H, s), 2.17 (6H, s), 2.49-2.60 (1H, m), 2.69-2.80 (1H, m), 3.69-3.84 (4H, m), 3.89-3.98 (1H, m), 4.23 (1H, dd, J=9.0, 5.9 Hz), 4.27-4.34 (2H, m), 4.72 (1H, t, J=9.0 Hz), 6.14 (1H, d, J=2.1 Hz), 6.18 (1H, dd, J=8.1, 2.1 Hz), 6.92-7.00 (2H, m), 7.22-7.79 (3H, m).

MS m/z 484 (M+H)⁺.

### Example 114

### [(3S)-6-({3-[2-(difluoromethoxy)-4,6-dimethylpyrimidin-5-yl]-2-methylbenzyl}amino)-2,3-dihydro-1-benzofuran-3-yl]acetic acid

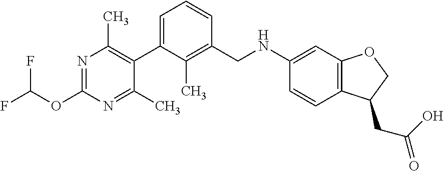

To a mixed solution of methyl [(3S)-6-({3-[2-(difluoromethoxy)-4,6-dimethylpyrimidin-5-yl]-2-methylbenzyl}amino)-2,3-dihydro-1-benzofuran-3-yl]acetate (0.102 g, 0.211 mmol) in methanol (0.5 mL) and tetrahydrofuran (1 mL) was added 1 M aqueous sodium hydroxide solution (0.5 mL), and the mixture was stirred at 50° C. for 1.5 hr. The reaction mixture was concentrated under reduced pressure, and the residue was dissolved in water. The solution was neutralized with 1 M hydrochloric acid, and the precipitated solid was purified by preparative HPLC to give the title compound (29.0 mg, yield 29%) as an orange non-crystalline powder.

¹H NMR (300 MHz, CDCl₃) δ 2.01 (3H, s), 2.17 (6H, s), 2.55-2.67 (1H, m), 2.74-2.86 (1H, m), 3.72-3.85 (1H, m), 4.21-4.35 (3H, m), 4.74 (1H, t, J=8.9 Hz), 6.15 (1H, d, J=1.9 Hz), 6.20 (1H, dd, J=8.0, 1.9 Hz), 6.91-7.03 (2H, m), 7.22-7.79 (3H, m).

MS m/z 470 (M+H)⁺.

### Example 115

### calcium [(3S)-6-({3-[2-(difluoromethoxy)-4,6-dimethylpyrimidin-5-yl]-2-methylbenzyl}amino)-2,3-dihydro-1-benzofuran-3-yl]acetate

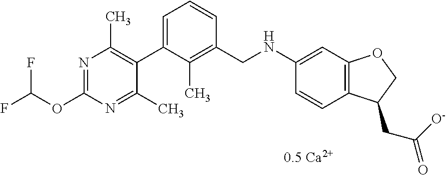

To a mixed solution of methyl [(3S)-6-({3-[2-(difluoromethoxy)-4,6-dimethylpyrimidin-5-yl]-2-methylbenzyl}amino)-2,3-dihydro-1-benzofuran-3-yl]acetate (0.328 g, 0.678 mmol) in methanol (1.5 mL) and tetrahydrofuran (3 mL) was added 1 M aqueous sodium hydroxide solution (1.3 mL), and the mixture was stirred at room temperature for 1 hr. The reaction mixture was neutralized with 1 M hydrochloric acid, and extracted with ethyl acetate. The extract was washed with saturated brine, dried over anhydrous sodium sulfate, and concentrated under reduced pressure. The residue was purified by preparative HPLC to give an oil. The oil was dissolved in water, and treated with 1 M aqueous sodium hydroxide solution (0.515 mL), and then 1 M aqueous calcium chloride solution (0.262 mL) was added. The precipitated solid was collected by filtration to give the title compound (0.147 g, yield 44%) as a beige powder.

¹H NMR (300 MHz, DMSO-d₆) δ 1.95 (3H, s), 2.03-2.21 (7H, m), 2.34-2.47 (1H, m), 3.51-3.65 (1H, m), 4.06 (1H, dd, J=8.5, 7.5 Hz), 4.21 (1H, d, J=5.1 Hz), 4.59 (1H, t, J=8.9 Hz), 5.90-6.04 (2H, m), 6.09 (1H, dd, J=8.1, 1.5 Hz), 6.91 (1H, d, J=8.1 Hz), 7.01 (1H, d, J=7.0 Hz), 7.25 (1H, t, J=7.5 Hz), 7.31-7.40 (1H, m), 7.47-8.02 (1H, m).

elemental analysis value for C₂₅H₂₄F₂N₃O₄S.0.5Ca²⁺.1.5H₂O

### Calculated: C, 58.24; H, 5.28; N, 8.15.

Found: C, 58.33; H, 5.25; N, 8.04.

### Example 116

### methyl {(3S)-6-[({2,2′,6′-trimethyl-4′-[3-(methylsulfonyl)propoxy]biphenyl-3-yl}methyl)amino]-2,3-dihydro-1-benzofuran-3-yl}acetate

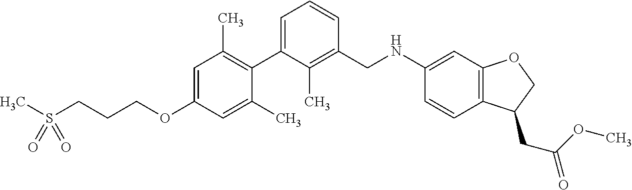

In the same manner as in Example 108, the title compound was obtained as a pale-yellow oil from {2,2′,6′-trimethyl-4′-[3-(methylsulfonyl)propoxy]biphenyl-3-yl}methanol and methyl [(3S)-6-{[(2-nitrophenyl)sulfonyl]amino}-2,3-dihydro-1-benzofuran-3-yl]acetate. yield 98% (2 steps).

¹H NMR (300 MHz, CDCl₃) δ 1.92 (6H, s), 1.96 (3H, s), 2.30-2.41 (2H, m), 2.48-2.60 (1H, m), 2.69-2.79 (1H, m), 2.97 (3H, s), 3.24-3.32 (2H, m), 3.69-3.83 (4H, m), 3.89 (1H, br s), 4.09-4.17 (2H, m), 4.19-4.31 (3H, m), 4.72 (1H, t, J=8.9 Hz), 6.13-6.22 (2H, m), 6.65 (2H, s), 6.92-6.98 (2H, m), 7.16-7.24 (1H, m), 7.28-7.34 (1H, m).

MS m/z 552 (M+H)⁺.

### Example 117

### {(3S)-6-[({2,2′,6′-trimethyl-4′-[3-(methylsulfonyl)propoxy]biphenyl-3-yl}methyl)amino]-2,3-dihydro-1-benzofuran-3-yl}acetic acid

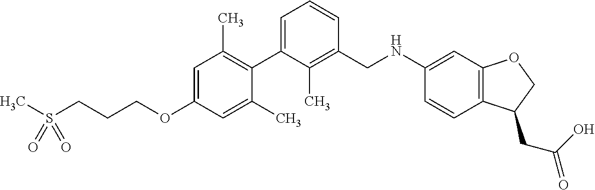

In the same manner as in Example 101, the title compound was obtained as beige crystals from methyl {(3S)-6-[({2,2′,6′-trimethyl-4′-[3-(methylsulfonyl)propoxy]biphenyl-3-yl}methyl)amino]-2,3-dihydro-1-benzofuran-3-yl}acetate. yield 73%.

¹H NMR (300 MHz, CDCl₃) δ 1.92 (6H, s), 1.96 (3H, s), 2.30-2.42 (2H, m), 2.54-2.66 (1H, m), 2.75-2.85 (1H, m), 2.97 (3H, s), 3.24-3.33 (2H, m), 3.73-3.84 (1H, m), 4.13 (2H, t, J=5.7 Hz), 4.22-4.31 (3H, m), 4.73 (1H, t, J=8.9 Hz), 6.14-6.23 (2H, m), 6.66 (2H, s), 6.93-7.01 (2H, m), 7.17-7.24 (1H, m), 7.29-7.34 (1H, m).

MS m/z 538 (M+H)⁺.

### Example 118

### ethyl (3-{[3-(6-fluoro-2-methyl-1H-benzimidazol-1-yl)-2-methylbenzyl]amino}-6,7-dihydro-5H-cyclopenta[b]pyridin-7-yl)acetate

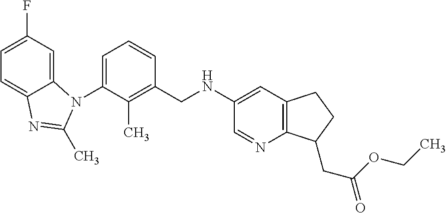

To a mixed solution of 3-(6-fluoro-2-methyl-1H-benzimidazol-1-yl)-2-methylbenzaldehyde (0.268 g, 1.00 mmol) synthesized, by an operation similar to Reference Example 126 and Reference Example 127, from methyl 3-(6-fluoro-2-methyl-1H-benzimidazol-1-yl)-2-methylbenzoate, ethyl (3-amino-6,7-dihydro-5H-cyclopenta[b]pyridin-7-yl)acetate (0.242 g, 1.10 mmol) synthesized according to the method described in WO2006/083612, and acetic acid (0.114 mL, 2.00 mmol) in acetonitrile (10 mL) and tetrahydrofuran (10 mL) was added under ice-cooling sodium triacetoxyborohydride (0.424 g, 2.00 mmol), and the mixture was warmed to room temperature and stirred for 12 hr. The reaction mixture was treated with water and saturated aqueous ammonium chloride solution, and extracted with ethyl acetate. The extract was washed successively with saturated aqueous sodium hydrogen carbonate and saturated brine, dried over anhydrous sodium sulfate, and concentrated under reduced pressure. The residue was purified by silica gel column chromatography (ethyl acetate:hexane=50:50-100:0, then methanol:ethyl acetate=0:100-10:90) to give the title compound (0.246 g, yield 52%) as a yellow viscous oil.

¹H NMR (300 MHz, CDCl₃) δ 1.26 (3H, t, J=7.2 Hz), 1.69-1.85 (1H, m), 1.95 (3H, s), 2.32-2.52 (5H, m), 2.74-2.91 (2H, m), 3.06 (1H, dd, J=15.5, 4.2 Hz), 3.43-3.57 (1H, m), 3.95 (1H, br s), 4.07-4.21 (2H, m), 4.39 (2H, s), 6.59 (1H, dd, J=8.6, 2.5 Hz), 6.82 (1H, d, J=2.7 Hz), 6.96-7.05 (1H, m), 7.16-7.22 (1H, m), 7.38 (1H, t, J=7.7 Hz), 7.55 (1H, d, J=7.7 Hz), 7.67 (1H, dd, J=8.6, 4.5 Hz), 7.86 (1H, d, J=2.7 Hz).

MS m/z 473 (M+H)⁺.

### Example 119

### (3-{[3-(6-fluoro-2-methyl-1H-benzimidazol-1-yl)-2-methylbenzyl]amino}-6,7-dihydro-5H-cyclopenta[b]pyridin-7-yl)acetic acid

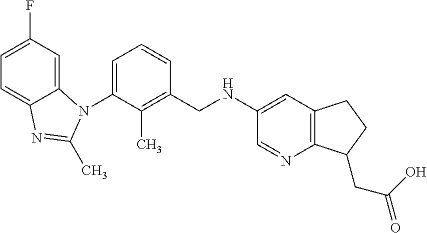

In the same manner as in Example 104, the title compound was obtained as a colorless powder from ethyl (3-{[3-(6-fluoro-2-methyl-1H-benzimidazol-1-yl)-2-methylbenzyl]amino}-6,7-dihydro-5H-cyclopenta[b]pyridin-7-yl)acetate. yield 67%.

¹H NMR (300 MHz, CDCl₃) δ 1.60-1.77 (1H, m), 1.95 (3H, s), 2.34-2.53 (4H, m), 2.61-2.72 (1H, m), 2.73-2.82 (1H, m), 2.83-2.94 (2H, m), 3.39-3.52 (1H, m), 4.20 (1H, br s), 4.42 (2H, s), 6.59 (1H, dd, J=8.7, 2.4 Hz), 6.93-7.0.6 (2H, m), 7.23 (1H, d, J=7.7 Hz), 7.40 (1H, t, J=7.7 Hz), 7.53 (1H, d, J=7.7 Hz), 7.68 (1H, dd, J=8.7, 4.7 Hz), 7.73 (1H, d, J=2.4 Hz).

MS m/z 445 (M+H)⁺.

### Example 120

### methyl [(3S)-6-{[3-(2,5-dimethylthiophen-3-yl)-2-fluorobenzyl]amino}-2,3-dihydro-1-benzofuran-3-yl]acetate

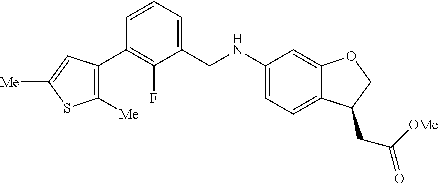

To a solution of [3-(2,5-dimethylthiophen-3-yl)-2-fluorophenyl]methanol (311 mg, 1.29 mmol), methyl [(3S)-6-{[(2-nitrophenyl)sulfonyl]amino}-2,3-dihydro-1-benzofuran-3-yl]acetate (455 mg, 1.29 mmol) and triphenylphosphine (677 mg, 2.58 mmol) in tetrahydrofuran (10 mL) was added diethyl azodicarboxylate (40% toluene solution, 1.52 mL, 3.87 mmol), and the mixture was stirred at room temperature for 3 hr. The reaction mixture was concentrated under reduced pressure, and the residue was purified by silica gel column chromatography (hexane:ethyl acetate=95:5-50:50) to give a pale-yellow solid (1.05 g). To a solution of the obtained solid (1.05 g) and mercaptoacetic acid (179 μL) in N,N-dimethylformamide (6.5 mL) was added lithium hydroxide monohydrate (217 mg, 5.16 mmol), and the mixture was stirred at room temperature for 2 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-70:30) to give the title compound (356 mg, yield 65%) as a pale-yellow oil.

¹H NMR (300 MHz, CDCl₃) δ 2.33 (3H, s), 2.45 (3H, s), 2.53 (1H, dd, J=16.2, 9.2 Hz), 2.73 (1H, dd, J=16.4, 5.3 Hz), 3.68-3.82 (4H, m), 4.08-4.17 (1H, m), 4.21 (1H, dd, J=9.0, 6.0 Hz), 4.35-4.44 (2H, m), 4.70 (1H, t, J=8.9 Hz), 6.12-6.21 (2H, m), 6.65 (1H, s), 6.93 (1H, d, J=7.9 Hz), 7.06-7.14 (1H, m), 7.14-7.22 (1H, m), 7.31 (1H, td, J=7.2, 2.0 Hz).

MS m/z 426.2 (M+H)⁺.

### Example 121

### [(3S)-6-{[3-(2,5-dimethylthiophen-3-yl)-2-fluorobenzyl]amino}-2,3-dihydro-1-benzofuran-3-yl]acetic acid

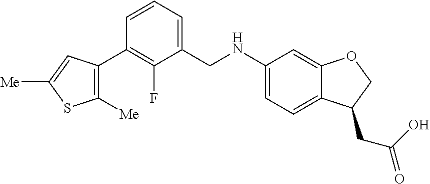

To a mixed solution of methyl [(3S)-6-{[3-(2,5-dimethylthiophen-3-yl)-2-fluorobenzyl]amino}-2,3-dihydro-1-benzofuran-3-yl]acetate (356 mg, 0.836 mmol) in tetrahydrofuran (5.2 mL) and methanol (2.6 mL) was added 1 M aqueous sodium hydroxide solution (2.51 mL), and the mixture was stirred at 50° C. for 1 hr. The reaction mixture was concentrated under reduced pressure, dissolved in distilled water, and cooled to 0° C. Thereto was slowly added 1 M hydrochloric acid to give a solid. The solid was collected by filtration, and washed with distilled water to give the title compound (301 mg, yield 87%) as a white solid.

¹H NMR (300 MHz, CDCl₃) δ 2.32 (3H, s), 2.44 (3H, s), 2.54 (1H, dd, J=16.5, 9.3 Hz), 2.73 (1H, dd, J=16.5, 5.2 Hz), 3.64-3.80 (1H, m), 4.21 (1H, dd, J=9.0, 6.0 Hz), 4.38 (2H, s), 4.67 (1H, t, J=8.9 Hz), 6.09-6.19 (2H, m), 6.64 (1H, s), 6.93 (1H, d, J=7.9 Hz), 7.03-7.13 (1H, m), 7.12-7.21 (1H, m), 7.24-7.34 (1H, m).

MS m/z 412 (M+H)⁺.

### Example 122

### methyl [(3S)-6-{[3-(2,5-dimethylthiophen-3-yl)-2-methylbenzyl]amino}-2,3-dihydro-1-benzofuran-3-yl]acetate

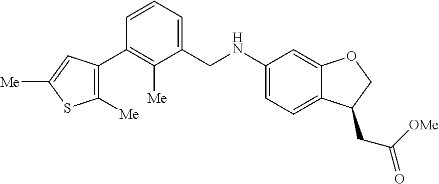

To a solution of methyl {(3S)-6-[(3-bromo-2-methylbenzyl)amino]-2,3-dihydro-1-benzofuran-3-yl}acetate (150 mg, 0.384 mmol), (2,5-dimethylthiophen-3-yl)boronic acid (120 mg, 0.769 mmol) and 2 M aqueous sodium carbonate solution (0.576 mL, 1.15 mmol) in toluene (2 mL) were added tris(dibenzylideneacetone)dipalladium (0) (10.5 mg. 0.012 mmol) and dicyclohexyl(2′,6′-dimethoxybiphenyl-2-yl)phosphane (18.9 mg, 0.046 mmol) under an argon atmosphere, and the mixture was stirred at 100° C. overnight. The reaction mixture was cooled to room temperature, filtered through celite, and concentrated under reduced pressure. The residue was purified by silica gel column chromatography (hexane:ethyl acetate=95:5-67:33) to give the title compound (140 mg, yield 86%) as a yellow oil.

¹H NMR (300 MHz, CDCl₃) δ 2.15 (3H, s), 2.18 (3H, s), 2.45 (3H, s), 2.54 (1H, dd, J=16.3, 9.1 Hz), 2.74 (1H, dd, J=16.3, 5.7 Hz), 3.71 (3H, s), 3.73-3.83 (1H, m), 3.91 (1H, br s), 4.23 (1H, dd, J=9.1, 6.1 Hz), 4.27 (2H, s), 4.72 (1H; t, J=8.9 Hz), 6.13-6.22 (2H, m), 6.50 (1H, s), 6.95 (1H, d, J=8.0 Hz), 7.05-7.12 (1H, m), 7.17 (1H, t, J=7.6 Hz), 7.23-7.34 (1H, m).

MS m/z 422 (M+H)⁺.

### Example 123

### [(3S)-6-{[3-(2,5-dimethylthiophen-3-yl)-2-methylbenzyl]amino}-2,3-dihydro-1-benzofuran-3-yl]acetic acid

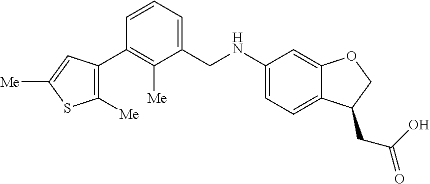

To a mixed solution of methyl [(3S)-6-{[3-(2,5-dimethylthiophen-3-yl)-2-methylbenzyl]amino}-2,3-dihydro-1-benzofuran-3-yl]acetate (140 mg, 0.331 mmol) in tetrahydrofuran (2 mL) and methanol (1 mL) was added 1 M aqueous sodium hydroxide solution (0.994 mL), and the mixture was stirred at 50° C. for 1 hr. The reaction mixture was concentrated under reduced pressure, dissolved in distilled water, and cooled to 0° C. Thereto was slowly added 1 M hydrochloric acid to give a solid. The solid was filtered, and washed with distilled water to give the title compound (115 mg, yield 85%) as a white solid.

¹H NMR (300 MHz, CDCl₃) δ 2.16 (3H, s), 2.18 (3H, s), 2.44 (3H, s), 2.60 (1H, dd, J=16.8, 9.4 Hz), 2.80 (1H, dd, J=16.6, 5.3 Hz), 3.71-3.85 (1H, m), 4.21-4.31 (3H, m), 4.73 (1H, t, J=8.9 Hz), 6.11-6.23 (2H, m), 6.50 (1H, d, J=0.9 Hz), 6.98 (1H, d, J=8.1 Hz), 7.07-7.13 (1H, m), 7.17 (1H, t, J=7.4 Hz), 7.27-7.33 (1H, m).

MS m/z 408 (M+H)⁺.

### Example 124

### [(3S)-6-{[3-(2-ethyl-6-fluoro-1H-benzimidazol-1-yl)-2-methylbenzyl]amino}-2,3-dihydro-1-benzofuran-3-yl]acetic acid

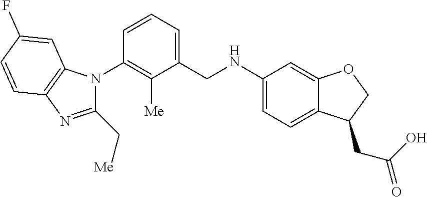

To a mixed solution of methyl [(3S)-6-{[3-(2-ethyl-6-fluoro-1H-benzimidazol-1-yl)-2-methylbenzyl]amino}-2,3-dihydro-1-benzofuran-3-yl]acetate (348 mg, 0.735 mmol) in tetrahydrofuran (4.6 mL) and methanol (2.3 mL) was added 1 M aqueous sodium hydroxide solution (2.21 mL), and the mixture was stirred at 50° C. for 1 hr. The reaction mixture was neutralized with 1 M hydrochloric acid, and diluted with distilled water, and the mixture was extracted with ethyl acetate. The extract was washed with saturated brine, dried over anhydrous magnesium sulfate, and concentrated under reduced pressure to give a solid. The solid was triturated with hexane-ethyl acetate to give the title compound (279 mg, yield 83%) as a white solid.

¹H NMR (300 MHz, CDCl₃) δ 1.32 (3H, s), 1.92 (3H, s), 2.55-2.72 (3H, m), 2.82 (1H, dd, J=16.7, 5.3 Hz), 3.75-3.89 (1H, m), 4.29 (1H, dd, J=9.5, 6.1 Hz), 4.36 (2H, s), 4.76 (1H, t, J=9.1 Hz), 6.12-6.25 (2H, m), 6.59 (1H, dd, J=8.5, 2.5 Hz), 6.96-7.06 (2H, m), 7.17 (1H, d, J=7.6 Hz), 7.36 (1H, t, J=7.8 Hz), 7.56 (1H, d, J=7.6 Hz), 7.74 (1H, dd, J=8.7, 4.9 Hz).

MS m/z 460 (M+H)⁺.

### Example 125

### sodium [(3S)-6-{[3-(2-ethyl-6-fluoro-1H-benzimidazol-1-yl)-2-methylbenzyl]amino}-2,3-dihydro-1-benzofuran-3-yl]acetate

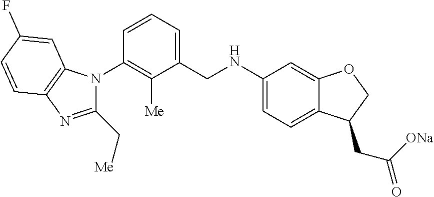

To a suspension of [(3S)-6-{[3-(2-ethyl-6-fluoro-1H-benzimidazol-1-yl)-2-methylbenzyl]amino}-2,3-dihydro-1-benzofuran-3-yl]acetic acid (220 mg, 0.479 mmol) in water (2.4 mL) was added 1 M aqueous sodium hydroxide solution (0.479 mL), and the mixture was stirred at room temperature. After dissolution of insoluble material, acetonitrile was added and the mixture was concentrated under reduced pressure to give the title compound (225 mg, yield 98%) as a white solid.

¹H NMR (300 MHz, DMSO-d₆) δ 1.21 (3H, t, J=7.5 Hz), 1.84 (3H, s), 1.95 (1H, dd, J=14.7, 10.2 Hz), 2.29 (1H, dd, J=14.7, 4.5 Hz), 2.53-2.64 (2H, m), 3.44-3.60 (1H, m), 4.05 (1H, t, J=7.9 Hz), 4.26 (2H, d, J=4.5 Hz), 4.58 (1H, t, J=8.7 Hz), 5.91-6.18 (3H, m), 6.69 (1H, d, J=7.2 Hz), 6.89 (1H, d, J=7.9 Hz), 7.07 (1H, t, J=8.3 Hz), 7.24-7.33 (1H, m), 7.39 (1H, t, J=7.5 Hz), 7.45-7.56 (1H, m), 7.67 (1H, dd, J=8.7, 4.9 Hz).

MS m/z 460 (M+H)⁺ (as free form).

### Example 126

### methyl [(3S)-6-{[2-methyl-3-(2-methyl-1H-benzimidazol-1-yl)benzyl]amino}-2,3-dihydro-1-benzofuran-3-yl]acetate

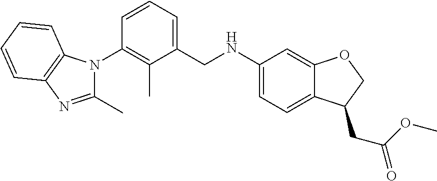

Methyl [(3S)-6-{[2-methyl-3-(2-methyl-1H-benzimidazol-1-yl)benzyl] [(2-nitrophenyl)sulfonyl]amino}-2,3-dihydro-1-benzofuran-3-yl]acetate (350 mg, 0.56 mmol), 2 M lithium hydroxide (1.1 mL) and sulfanylacetic acid (103 mg, 1.1 mmol) were mixed in N,N-dimethylformamide (10 mL), and the mixture was stirred overnight at room temperature, diluted with saturated brine, and extracted with ethyl acetate. The extract was dried over anhydrous magnesium sulfate, and the volatile component was evaporated under reduced pressure. The residue was purified by silica gel column chromatography (hexane:ethyl acetate=50:50-0:100) to give the title compound (35 mg, yield 14%) as a colorless solid.

¹H NMR (300 MHz, DMSO-d₆) δ 1.84 (3H, s), 2.30 (3H, s), 2.52-2.60 (1H, m), 2.65-2.77 (1H, m), 3.55-3.69 (4H, m), 4.11 (1H, dd, J=9.1, 6.4 Hz), 4.28 (2H, d, J=5.3 Hz), 4.59 (1H, t, J=9.1 Hz), 6.06 (1H, d, J=1.9 Hz), 6.11-6.19 (2H, m), 6.89 (2H, t, J=7.4 Hz), 7.11-7.30 (3H, m), 7.39 (1H, t, J=7.6 Hz), 7.46-7.52 (1H, m), 7.63 (1H, d, J=7.2 Hz).

### Example 127

### [(3S)-6-{[2-methyl-3-(2-methyl-1H-benzimidazol-1-yl)benzyl]amino}-2,3-dihydro-1-benzofuran-3-yl]acetic acid

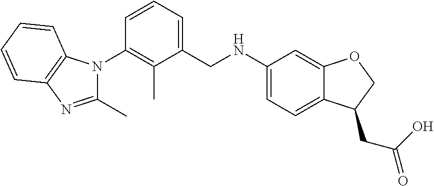

Methyl [(3S)-6-{[2-methyl-3-(2-methyl-1H-benzimidazol-1-yl)benzyl]amino}-2,3-dihydro-1-benzofuran-3-yl]acetate (35 mg) was dissolved in tetrahydrofuran (2 mL) and methanol (2 mL), 8N aqueous sodium hydroxide solution (0.1 mL) was added, and the mixture was stirred at room temperature for 3 hr. The volatile component was evaporated under reduced pressure, and 1 M aqueous hydrochloric acid solution (0.8 mL) was added. The mixture was diluted with saturated brine, and extracted with ethyl acetate. The extract was dried over anhydrous magnesium sulfate and the volatile component was evaporated under reduced pressure to give a white solid. The obtained solid was crystallized from ethyl acetate-hexane to give the title compound (20 mg, yield 59%) as a white solid.

¹H NMR (300 MHz, DMSO-d₆) δ 1.84 (3H, s), 2.30 (3H, s), 2.36-2.44 (1H, m), 2.56-2.67 (1H, m), 3.53-3.66 (1H, m), 4.05-4.14 (1H, m), 4.28 (2H, d, J=5.3 Hz), 4.60 (1H, t, J=9.1 Hz), 6.02-6.18 (3H, m), 6.85-6.94 (2H, m), 7.11-7.30 (3H, m), 7.39 (1H, t, J=8.0 Hz), 7.50 (1H, d, J=7.6 Hz), 7.63 (1H, d, J=7.2 Hz).

MS m/z 428 (M+H)⁺.

### Example 128

### methyl [(3S)-6-({2-methyl-3-[2-methyl-6-(trifluoromethyl)-1H-benzimidazol-1-yl]benzyl}amino)-2,3-dihydro-1-benzofuran-3-yl]acetate

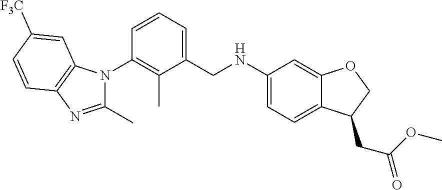

To a solution of methyl 2-methyl-3-[2-methyl-6-(trifluoromethyl)-1H-benzimidazol-1-yl]benzoate (140 mg, 0.40 mmol) in tetrahydrofuran (10 mL) was added lithium aluminum hydride (31 mg, 0.80 mmol), and the mixture was stirred at 0° C. for 1 hr. Sodium sulfate 10 hydrate was added, and then ethyl acetate was added, and the mixture was filtered through a celite pad. The filtrate was concentrated, and the obtained residue was purified by silica gel column chromatography (hexane:ethyl acetate=50:50-0:100) to give {2-methyl-3-[2-methyl-6-(trifluoromethyl)-1H-benzimidazol-1-yl]phenyl}methanol (99 mg, 0.31 mmol) as a pale-yellow oil. To a solution of the obtained compound in acetonitrile (5 mL) was added Dess-Martin reagent (147 mg, 0.47 mmol) at 0° C., and the mixture was stirred at room temperature for 1 hr. Aqueous sodium thiosulfate solution was added, and the mixture was extracted with ethyl acetate. The extract was washed with saturated brine, dried over anhydrous magnesium sulfate, and the volatile component was evaporated under reduced pressure. To a solution of the obtained residue, methyl [(3S)-6-amino-2,3-dihydro-1-benzofuran-3-yl]acetate (96 mg, 0.47 mmol) and acetic acid (0.5 mL) in acetonitrile (5 mL) was added sodium triacetoxyborohydride (263 mg, 1.24 mmol), and the mixture was stirred overnight at room temperature. The mixture was diluted with aqueous sodium bicarbonate solution and extracted with ethyl acetate. The extract was washed with saturated brine, dried over anhydrous magnesium sulfate, and the volatile component was evaporated under reduced pressure. The obtained residue was purified by silica gel column chromatography (hexane:ethyl acetate=70:30-20:80) to give the title compound (96 mg, yield 61%).

¹H NMR (300 MHz, DMSO-d₆) δ 1.84 (3H, s), 2.35 (3H, s), 2.52-2.59 (1H, m), 2.66-2.76 (1H, m), 3.58-3.68 (4H, m), 4.11 (1H, dd, J=9.0, 6.4 Hz), 4.29 (2H, d, J=5.5 Hz), 4.59 (1H, t, J=8.9 Hz), 6.06 (1H, d, J=1.7 Hz), 6.12-6.19 (2H, m), 6.90 (1H, d, J=8.1 Hz), 7.17 (1H, s), 7.34 (1H, d, J=6.6 Hz), 7.42 (1H, t, J=7.6 Hz), 7.50-7.59 (2H, m), 7.86 (1H, d, J=8.3 Hz).

### Example 129

### methyl [(3S)-6-({2-methyl-3-[2-methyl-5-(trifluoromethyl)-1H-benzimidazol-1-yl]benzyl}amino)-2,3-dihydro-1-benzofuran-3-yl]acetate

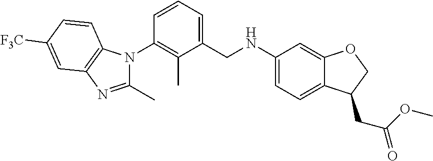

In the same manner as in Example 128, the title compound (67 mg, 48%) was obtained from methyl 2-methyl-3-[2-methyl-5-(trifluoromethyl)-1H-benzimidazol-1-yl]benzoate (140 mg).

¹H NMR (300 MHz, DMSO-d₆) δ 1.84 (3H, s), 2.35 (3H, s), 2.52-2.59 (1H, m), 2.66-2.76 (1H, m), 3.56-3.69 (4H, m), 4.07-4.16 (1H, m), 4.29 (2H, d, J=5.7 Hz), 4.60 (1H, t, J=8.9 Hz), 6.00-6.21 (3H, m), 6.90 (1H, d, J=8.3 Hz), 7.09 (1H, d, J=8.5 Hz), 7.30-7.36 (1H, m), 7.41 (1H, t, J=7.7 Hz), 7.47-7.57 (2H, m), 8.01 (1H, s).

### Example 130

### [(3S)-6-({2-methyl-3-[2-methyl-6-(trifluoromethyl)-1H-benzimidazol-1-yl]benzyl}amino)-2,3-dihydro-1-benzofuran-3-yl]acetic acid

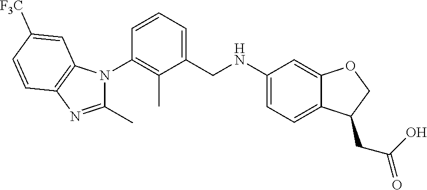

Methyl [(3S)-6-({2-methyl-3-[2-methyl-6-(trifluoromethyl)-1H-benzimidazol-1-yl]benzyl}amino)-2,3-dihydro-1-benzofuran-3-yl]acetate (96 mg, 0.19 mmol) was dissolved in tetrahydrofuran (2 mL) and methanol (2 mL), 8 M aqueous sodium hydroxide solution (0.25 mL) was added, and the mixture was stirred at room temperature for 4 hr. The volatile component was evaporated under reduced pressure, and 1 M aqueous hydrochloric acid solution (2.0 mL) was added. The mixture was diluted with aqueous sodium bicarbonate solution, and extracted with ethyl acetate. The extract was washed with saturated brine and dried over anhydrous magnesium sulfate and the volatile component was evaporated under reduced pressure to give the title compound (60 mg) as a white solid.

¹H NMR (300 MHz, DMSO-d₆) δ 1.85 (3H, s), 2.31-2.45 (4H, m), 2.56-2.68 (1H, m), 3.55-3.66 (1H, m), 4.06-4.16 (1H, m), 4.25-4.36 (2H, m), 4.60 (1H, t, J=8.9 Hz), 6.01-6.23 (3H, m), 6.93 (1H, d, J=8.0 Hz), 7.17 (1H, s), 7.30-7.47 (2H, m), 7.55 (2H, t, J=7.8 Hz), 7.86 (1H, d, J=8.7 Hz).

MS m/z 496 (M+H)⁺.

### Example 131

### [(3S)-6-({2-methyl-3-[2-methyl-5-(trifluoromethyl)-1H-benzimidazol-1-yl]benzyl}amino)-2,3-dihydro-1-benzofuran-3-yl]acetic acid

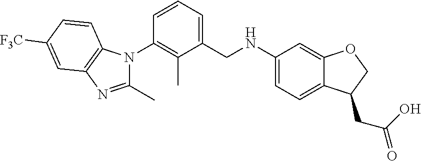

Methyl [(3S)-6-({2-methyl-3-[2-methyl-5-(trifluoromethyl)-1H-benzimidazol-1-yl]benzyl}amino)-2,3-dihydro-1-benzofuran-3-yl]acetate (67 mg, 0.13 mmol) was dissolved in tetrahydrofuran (2 mL) and methanol (2 mL), 8N aqueous sodium hydroxide solution (0.25 mL) was added, and the mixture was stirred at room temperature for 4 hr. The volatile component was evaporated under reduced pressure, and 1 M aqueous hydrochloric acid solution (2.0 mL) was added. The mixture was diluted with aqueous sodium bicarbonate solution, and extracted with ethyl acetate. The extract was washed with saturated brine and dried over anhydrous magnesium sulfate and the volatile component was evaporated under reduced pressure to give the title compound (60 mg) as a white solid.

¹H NMR (300 MHz, DMSO-d₆) δ 1.84 (3H, s), 2.24-2.40 (4H, m), 2.53-2.63 (1H, m), 3.53-3.62 (1H, m), 4.04-4.13 (1H, m), 4.29 (2H, d, J=5.7 Hz), 4.59 (1H, t, J=8.9 Hz), 6.02-6.17 (3H, m), 6.92 (1H, d, J=8.3 Hz), 7.09 (1H, d, J=8.3 Hz), 7.30-7.35 (1H, m), 7.41 (1H, t, J=7.8 Hz), 7.47-7.56 (2H, m), 8.01 (1H, s).

MS m/z 496 (M+H)⁺.

### Example 132

### methyl [(3S)-6-{[2-fluoro-3-(2-methyl-1H-benzimidazol-1-yl)benzyl]amino}-2,3-dihydro-1-benzofuran-3-yl]acetate

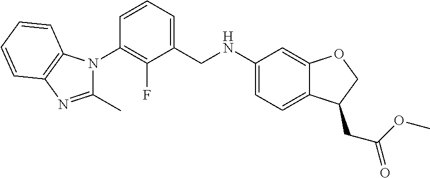

In the same manner as in Example 128, the title compound (yield 76%) was synthesized from methyl 2-fluoro-3-(2-methyl-1H-benzimidazol-1-yl)benzoate.

¹H NMR (300 MHz, DMSO-d₆) δ 2.39 (3H, s), 2.52-2.58 (1H, m), 2.65-2.76 (1H, m), 3.56-3.68 (4H, m), 4.11 (1H, dd, J=8.9, 6.6 Hz), 4.37 (2H, d, J=6.4 Hz), 4.59 (1H, t, J=8.9 Hz), 6.07 (1H, d, J=1.9 Hz), 6.14 (1H, dd, J=8.3, 1.9 Hz), 6.28 (1H, t, J=6.1 Hz), 6.89 (1H, d, J=8.3 Hz), 7.05 (1H, d, J=7.6 Hz), 7.16-7.28 (2H, m), 7.36-7.44 (1H, m), 7.50-7.67 (3H, m).

### Example 133

### [(3S)-6-{[2-fluoro-3-(2-methyl-1H-benzimidazol-1-yl)benzyl]amino}-2,3-dihydro-1-benzofuran-3-yl]acetic acid

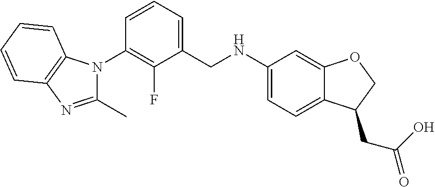

Methyl [(3S)-6-{[2-fluoro-3-(2-methyl-1H-benzimidazol-1-yl)benzyl]amino}-2,3-dihydro-1-benzofuran-3-yl]acetate (145 mg, 0.32 mmol) was dissolved in tetrahydrofuran (2 mL) and methanol (2 mL), 8 M aqueous sodium hydroxide solution (0.25 mL) was added, and the mixture was stirred at room temperature for 2 hr. The volatile component was evaporated under reduced pressure, 1 M aqueous hydrochloric acid solution (2.0 mL) was added, and the mixture was extracted with ethyl acetate. The extract was washed with saturated brine and dried over anhydrous magnesium sulfate and the volatile component was evaporated under reduced pressure to give the title compound (130 mg, yield 94%) as a white solid.

¹H NMR (300 MHz, DMSO-d₆) δ 2.36-2.46 (4H, m), 2.62 (1H, dd, J=16.5, 5.5 Hz), 3.53-3.66 (1H, m), 4.06-4.14 (1H, m), 4.37 (2H, d, J=5.7 Hz), 4.60 (1H, t, J=8.9 Hz), 6.06 (1H, d, J=1.9 Hz), 6.14 (1H, dd, J=8.1, 2.1 Hz), 6.26 (1H, t, J=6.2 Hz), 6.92 (1H, d, J=8.3 Hz), 7.05 (1H, d, J=7.6 Hz), 7.16-7.28 (2H, m), 7.40 (1H, t, J=7.8 Hz), 7.49-7.68 (3H, m), 12.30 (1H, br s).

MS m/z 432 (M+H)⁺.

### Example 134

### methyl [(3S)-6-{[3-(6-fluoro-2-methyl-1H-benzimidazol-1-yl)-2-methylbenzyl]amino}-2,3-dihydro-1-benzofuran-3-yl]acetate

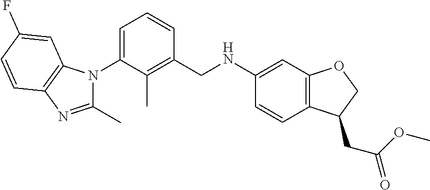

Methyl 3-(6-fluoro-2-methyl-1H-benzimidazol-1-yl)-2-methylbenzoate (850 mg, 2.85 mmol) was dissolved in tetrahydrofuran (10 mL), lithium aluminum hydride (216 mg, 5.70 mmol) was added at 0° C., and the mixture was stirred at room temperature for 30 min. Sodium sulfate 10 hydrate was added, and then ethyl acetate was added, and the mixture was filtered through a celite pad. The filtrate was concentrated to give a white solid (730 mg). To a solution of the obtained residue (270 mg) in acetonitrile (10 mL) was added a Dess-Martin reagent (636 mg, 1.5 mmol), and the mixture was stirred at room temperature for 1 hr. An aqueous sodium thiosulfate solution was added, and the mixture was extracted with ethyl acetate. The extract was washed with aqueous sodium bicarbonate solution and saturated brine, and dried over anhydrous magnesium sulfate, and the volatile component was evaporated under reduced pressure. To a solution of the obtained residue (300 mg), methyl [(3S)-6-amino-2,3-dihydro-1-benzofuran-3-yl]acetate (255 mg, 1.23 mmol) and acetic acid (0.5 mL) in acetonitrile (10 mL) was added sodium triacetoxyborohydride (947 mg, 4.47 mmol), and the mixture was stirred at room temperature for 2 hr. The mixture was diluted with aqueous sodium bicarbonate solution and extracted with ethyl acetate. The extract was washed with saturated brine, dried over anhydrous magnesium sulfate, and the volatile component was evaporated under reduced pressure. The obtained residue was purified by silica gel column chromatography (hexane:ethyl acetate=80:30-20:80) to give the title compound (300 mg, yield 61%).

MS m/z 460 (M+H)⁺.

### Example 135

### methyl [(3S)-6-{[3-(5-fluoro-2-methyl-1H-benzimidazol-1-yl)-2-methylbenzyl]amino}-2,3-dihydro-1-benzofuran-3-yl]acetate

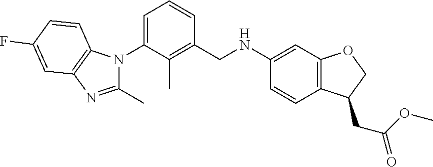

In the same manner as in Reference Examples 104, 105, 106 and Example 134, the title compound was synthesized from 1-bromo-4-fluoro-2-nitrobenzene.

MS m/z 460 (M+H)⁺.

### Example 136

### [(3S)-6-{[3-(6-fluoro-2-methyl-1H-benzimidazol-1-yl)-2-methylbenzyl]amino}-2,3-dihydro-1-benzofuran-3-yl]acetic acid

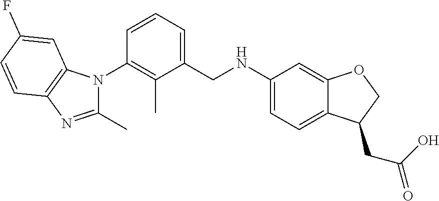

Methyl [(3S)-6-{[3-(6-fluoro-2-methyl-1H-benzimidazol-1-yl)-2-methylbenzyl]amino}-2,3-dihydro-1-benzofuran-3-yl]acetate (300 mg, 0.65 mmol) was dissolved in tetrahydrofuran (5 mL) and methanol (5 mL), 8 M aqueous sodium hydroxide solution (0.5 mL) was added, and the mixture was stirred at 50° C. for 1 hr. Water (10 mL) was added, and the volatile component was evaporated under reduced pressure. To the obtained residue was added 1 M aqueous hydrochloric acid solution (4.0 mL) and the precipitate was collected by filtration to give the title compound (180 mg, yield 62%) as a white solid.

¹H NMR (300 MHz, DMSO-d₆) δ 1.84 (3H, s), 2.29 (3H, s), 2.34-2.44 (1H, m), 2.55-2.65 (1H, m), 3.53-3.65 (1H, m), 4.04-4.13 (1H, m), 4.28 (2H, d, J=4.9 Hz), 4.59 (1H, t, J=8.9 Hz), 6.02-6.18 (3H, m), 6.70 (1H, dd, J=8.9, 2.5 Hz), 6.92 (1H, d, J=8.0 Hz), 7.06 (1H, dd, J=18.7, 2.5 Hz), 7.25-7.31 (1H, m), 7.39 (1H, t, J=7.8 Hz), 7.50 (1H, d, J=6.8 Hz), 7.64 (1H, dd, J=8.9, 4.7 Hz).

MS m/z 446 (M+H)⁺.

### Example 137

### sodium [(3S)-6-{[3-(6-fluoro-2-methyl-1H-benzimidazol-1-yl)-2-methylbenzyl]amino}-2,3-dihydro-1-benzofuran-3-yl]acetate

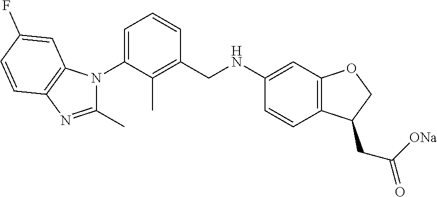

[(3S)-6-{[3-(6-Fluoro-2-methyl-1H-benzimidazol-1-yl)-2-methylbenzyl]amino}-2,3-dihydro-1-benzofuran-3-yl]acetic acid (100 mg, 0.22 mmol) was suspended in water (5 mL), 1 M aqueous sodium hydroxide solution (0.22 mL) was added, and acetonitrile (20 mL) was further added. The volatile component was evaporated under reduced pressure to give the title compound (100 mg, yield 97%) as a white solid.

¹H NMR (300 MHz, DMSO-d₆) δ 1.85 (3H, s), 1.93 (1H, dd, J=14.9, 10.0 Hz), 2.23-2.32 (4H, m), 3.44-3.55 (1H, m), 4.04 (1H, dd, J=8.7, 7.2 Hz), 4.26 (2H, d, J=5.7 Hz), 4.57 (1H, t, J=8.9 Hz), 5.96-6.03 (2H, m), 6.10 (1H, dd, J=8.1, 2.1 Hz), 6.70 (1H, dd, J=8.9, 2.4 Hz), 6.88 (1H, d, J=8.3 Hz), 7.01-7.10 (1H, m), 7.25-7.30 (1H, m), 7.39 (1H, t, J=7.7 Hz), 7.51 (1H, d, J=6.8 Hz), 7.64 (1H, dd, J=8.7, 4.9 Hz).

### Example 138

### [(3S)-6-{[3-(5-fluoro-2-methyl-1H-benzimidazol-1-yl)-2-methylbenzyl]amino}-2,3-dihydro-1-benzofuran-3-yl]acetic acid

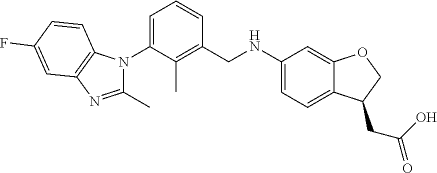

Methyl [(3S)-6-{[3-(5-fluoro-2-methyl-1H-benzimidazol-1-yl)-2-methylbenzyl]amino}-2,3-dihydro-1-benzofuran-3-yl]acetate (600 mg, 1.3 mmol) was dissolved in tetrahydrofuran (5 mL) and methanol (5 mL), 8 M aqueous sodium hydroxide solution (0.5 mL) was added, and the mixture was stirred at 50° C. for 1 hr. Water (10 mL) was added, and the volatile component was evaporated under reduced pressure. To the residue was added 1 M aqueous hydrochloric acid solution (4.0 mL) and the precipitate was collected by filtration to give the title compound (490 mg, yield 85%) as a white solid.

¹H NMR (300 MHz, DMSO-d₆) δ 1.84 (3H, s), 2.30 (3H, s), 2.34-2.44 (1H, m), 2.55-2.65 (1H, m), 3.54-3.64 (1H, m), 4.04-4.13 (1H, m), 4.28 (2H, d, J=5.3 Hz), 4.59 (1H, t, J=8.9 Hz), 6.02-6.17 (3H, m), 6.83-6.94 (2H, m), 7.00 (1H, dd, J=9.7, 2.5 Hz), 7.25-7.31 (1H, m), 7.35-7.53 (3H, m).

MS m/z 446 (M+H)⁺.

### Example 139

### methyl [(3S)-6-{[3-(2-ethoxy-1H-benzimidazol-1-yl)-2-methylbenzyl]amino}-2,3-dihydro-1-benzofuran-3-yl]acetate

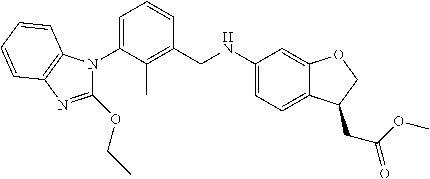

The title compound was synthesized in the same manner as in Example 134 and using methyl 3-(2-ethoxy-1H-benzimidazol-1-yl)-2-methylbenzoate.

MS m/z 472 (M+H)⁺.

### Example 140

### [(3S)-6-{[3-(2-ethoxy-1H-benzimidazol-1-yl)-2-methylbenzyl]amino}-2,3-dihydro-1-benzofuran-3-yl]acetic acid

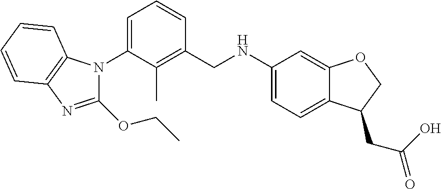

Methyl [(3S)-6-{[3-(2-ethoxy-1H-benzimidazol-1-yl)-2-methylbenzyl]amino}-2,3-dihydro-1-benzofuran-3-yl]acetate (220 mg, 0.47 mmol) was dissolved in tetrahydrofuran (5 mL) and methanol (5 mL), 8 N aqueous sodium hydroxide solution (0.5 mL) was added, and the mixture was stirred overnight at room temperature. Water (10 mL) was added, and the volatile component was evaporated under reduced pressure. To the residue was added 1 M aqueous hydrochloric acid solution (4.0 mL) and the precipitate was collected by filtration to give the title compound (75 mg, yield 35%).

¹H NMR (300 MHz, DMSO-d₆) δ 1.33 (3H, t, J=7.2 Hz), 1.94 (3H, s), 2.36-2.46 (1H, m), 2.57-2.67 (1H, m), 3.53-3.66 (1H, m), 4.09 (1H, dd, J=8.7, 6.8 Hz), 4.27 (2H, d, J=5.3 Hz), 4.48-4.64 (3H, m), 6.03-6.18 (3H, m), 6.81 (1H, d, J=7.6 Hz), 6.92 (1H, d, J=8.0 Hz), 7.03-7.10 (1H, m), 7.12-7.19 (1H, m), 7.22-7.28 (1H, m), 7.34 (1H, t, J=7.6 Hz), 7.42-7.53 (2H, m).

MS m/z 458 (M+H)⁺.

### Example 141

### methyl [(3S)-6-{[3-(2-methoxy-1H-benzimidazol-1-yl)-2-methylbenzyl]amino}-2,3-dihydro-1-benzofuran-3-yl]acetate

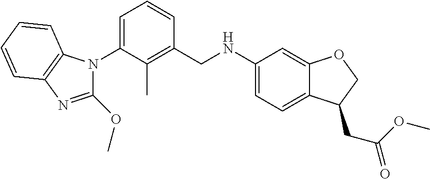

In the same manner as in Example 139, the title compound was synthesized.

¹H NMR (300 MHz, DMSO-d₆) δ 1.92 (3H, s), 2.51-2.60 (1H, m), 2.66-2.75 (1H, m), 3.58-3.68 (4H, m), 4.07-4.14 (4H, m), 4.27 (2H, d, J=5.7 Hz), 4.59 (1H, t, J=8.9 Hz), 6.06 (1H, d, J=1.9 Hz), 6.10-6.19 (2H, m), 6.82 (1H, d, J=7.6 Hz), 6.89 (1H, d, J=8.0 Hz), 7.04-7.11 (1H, m), 7.13-7.20 (1H, m), 7.23-7.28 (1H, m), 7.34 (1H, t, J=7.6 Hz), 7.45 (1H, d, J=7.6 Hz), 7.52 (1H, d, J=7.6 Hz).

### Example 142

### [(3S)-6-{[3-(2-methoxy-1H-benzimidazol-1-yl)-2-methylbenzyl]amino}-2,3-dihydro-1-benzofuran-3-yl]acetic acid

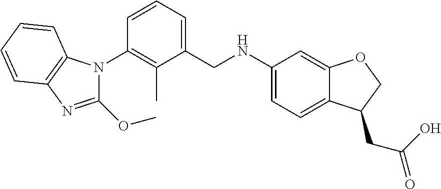

In the same manner as in Example 104, the title compound was synthesized from methyl [(3S)-6-{[3-(2-methoxy-1H-benzimidazol-1-yl)-2-methylbenzyl]amino}-2,3-dihydro-1-benzofuran-3-yl]acetate.

¹H NMR (300 MHz, DMSO-d₆) δ 1.92 (3H, s), 2.38 (1H, dd, J=16.3, 9.1 Hz), 2.56-2.65 (1H, m), 3.51-3.64 (1H, m), 4.03-4.13 (4H, m), 4.26 (2H, d, J=4.2 Hz), 4.59 (1H, t, J=9.1 Hz), 6.02-6.17 (3H, m), 6.82 (1H, d, J=7.6 Hz), 6.92 (1H, d, J=8.0 Hz), 7.05-7.20 (2H, m), 7.23-7.29 (1H, m), 7.34 (1H, t, J=7.8 Hz), 7.45 (1H, d, J=6.8 Hz), 7.52 (1H, d, J=7.2 Hz).

MS m/z 444 (M+H)⁺.

### Example 143

### methyl [(3S)-6-({3-[5-methoxy-2-(trifluoromethyl)-1H-benzimidazol-1-yl]-2-methylbenzyl}amino)-2,3-dihydro-1-benzofuran-3-yl]acetate

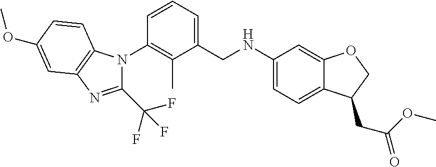

In the same manner as in Example 134, the title compound was synthesized from methyl 3-[5-methoxy-2-(trifluoromethyl)-1H-benzimidazol-1-yl]-2-methylbenzoate.

¹H NMR (300 MHz, DMSO-d₆) δ 1.82 (3H, s), 2.52-2.60 (1H, m), 2.66-2.76 (1H, m), 3.57-3.70 (4H, m), 3.85 (3H, s), 4.11 (1H, dd, J=8.9, 6.5 Hz), 4.28 (2H, d, J=5.8 Hz), 4.59 (1H, t, J=8.9 Hz), 6.04 (1H, d, J=1.9 Hz), 6.10-6.20 (2H, m), 6.86-6.98 (2H, m), 7.09 (1H, dd, J=9.0, 2.3 Hz), 7.34-7.47 (3H, m), 7.53 (1H, dd, J=6.7, 2.5 Hz).

### Example 144

### (3S)-6-({3-[5-methoxy-2-(trifluoromethyl)-1H-benzimidazol-1-yl]-2-methylbenzyl}amino)-2,3-dihydro-1-benzofuran-3-yl]acetic acid

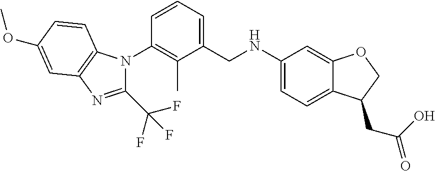

In the same manner as in Example 136, the title compound was synthesized using methyl [(3S)-6-({3-[5-methoxy-2-(trifluoromethyl)-1H-benzimidazol-1-yl]-2-methylbenzyl}amino)-2′,3-dihydro-1-benzofuran-3-yl]acetate.

¹H NMR (300 MHz, DMSO-d₆) δ 1.82 (3H, s), 2.34 (1H, dd, J=16.1, 9.3 Hz), 2.57 (1H, dd, J=16.1, 5.5 Hz), 3.52-3.63 (1H, m), 3.84 (3H, s), 4.08 (1H, dd, J=8.7, 6.8 Hz), 4.28 (2H, d, J=5.7 Hz), 4.59 (1H, t, J=8.9 Hz), 6.02 (1H, d, J=1.9 Hz), 6.10-6.16 (2H, m), 6.88-6.98 (2H, m), 7.06-7.11 (1H, m), 7.33-7.46 (3H, m), 7.53 (1H, dd, J=6.8, 2.3 Hz).

MS m/z 512 (M+H)⁺.

### Example 145

### methyl {(3S)-6-[(2-methyl-3-{2-[(2S)-tetrahydrofuran-2-yl]-1H-benzimidazol-1-yl}benzyl)amino]-2,3-dihydro-1-benzofuran-3-yl}acetate

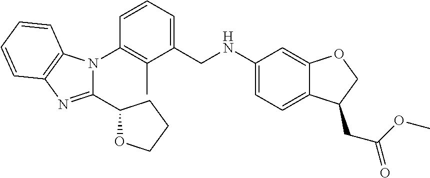

In the same manner as in Reference Example 103 and Example 134, the title compound was synthesized from methyl 2-methyl-3-[(2-{[(2S)-tetrahydrofuran-2-ylcarbonyl]amino}phenyl)amino]benzoate.

¹H NMR (300 MHz, DMSO-d₆) δ 1.75-1.93 (4H, m), 2.01-2.23 (2H, m), 2.42-2.47 (1H, m), 2.53-2.60 (1H, m), 2.66-2.77 (1H, m), 3.59-3.76 (6H, m), 4.11 (1H, dd, J=9.0, 6.6 Hz), 4.28 (2H, br s), 4.59 (1H, t, J=8.9 Hz), 4.72-4.88 (1H, m), 6.05 (1H, d, J=1.7 Hz), 6.11-6.21 (2H, m), 6.86-6.93 (2H, m), 7.18-7.31 (3H, m), 7.32-7.42 (1H, m), 7.45-7.51 (1H, m), 7.73 (1H, d, J=7.0 Hz).

### Example 146

### {(3S)-6-[(2-methyl-3-{2-[(2S)-tetrahydrofuran-2-yl]-1H-benzimidazol-1-yl}benzyl)amino]-2,3-dihydro-1-benzofuran-3-yl}acetic acid

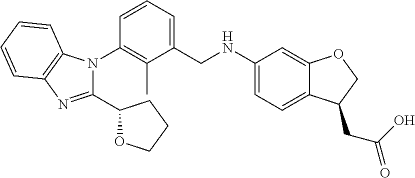

In the same manner as in Example 136, the title compound was obtained as a white solid from methyl {(3S)-6-[(2-methyl-3-{2-[(2S)-tetrahydrofuran-2-yl]-1H-benzimidazol-1-yl}benzyl)amino]-2,3-dihydro-1-benzofuran-3-yl}acetate.

¹H NMR (300 MHz, DMSO-d₆) δ 1.73-2.47 (8H, m), 2.57-2.71 (1H, m), 3.51-3.77 (3H, m), 4.09 (1H, dd, J=8.9, 6.6 Hz), 4.28 (2H, br s), 4.60 (1H, t, J=8.9 Hz), 4.70-4.92 (1H, m), 6.00-6.20 (3H, m), 6.86-6.97 (2H, m), 7.17-7.41 (4H, m), 7.49 (1H, d, J=7.6 Hz), 7.73 (1H, d, J=6.8 Hz).

MS m/z 484 (M+H)⁺.

### Example 147

### {(3S)-6-[(2-methyl-3-{2-[(2R)-tetrahydrofuran-2-yl]-1H-benzimidazol-1-yl}benzyl)amino]-2,3-dihydro-1-benzofuran-3-yl}acetic acid

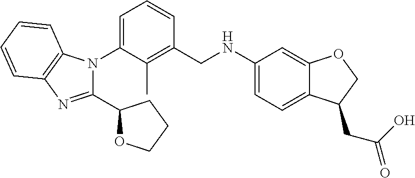

In the same manner as in Example 146, the title compound was obtained as a white solid.

¹H NMR (300 MHz, DMSO-d₆) δ 1.76-2.29 (6H, m), 2.36-2.46 (2H, m), 2.57-2.68 (1H, m), 3.53-3.77 (3H, m), 4.09 (1H, dd, J=8.9, 6.6 Hz), 4.27 (2H, d, J=4.2 Hz), 4.60 (1H, t, J=8.9 Hz), 4.71-4.89 (1H, m), 6.00-6.17 (3H, m), 6.86-6.96 (2H, m), 7.18-7.31 (3H, m), 7.32-7.42 (1H, m), 7.49 (1H, d, J=7.6 Hz), 7.73 (1H, d, J=6.8 Hz).

MS m/z 484 (M+H)⁺.

### Example 148

### methyl [(3S)-6-{[3-(2-cyclopropyl-5-methoxy-1H-benzimidazol-1-yl)-2-methylbenzyl]amino}-2,3-dihydro-1-benzofuran-3-yl]acetate

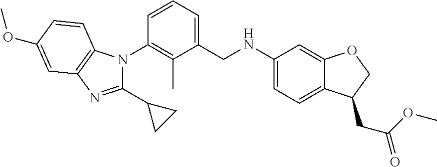

In the same manner as in Reference Example 106 and Example 134, the title compound was obtained using methyl 3-[(2-amino-4-methoxyphenyl)amino]-2-methylbenzoate and cyclopropanecarbonyl chloride.

¹H NMR (300 MHz, DMSO-d₆) δ 0.92-1.11 (4H, m), 1.52-1.63 (1H, m), 1.91 (3H, s), 2.51-2.59 (1H, m), 2.66-2.75 (1H, m), 3.56-3.69 (4H, m), 3.77 (3H, s), 4.11 (1H, dd, J=8.9, 6.6 Hz), 4.29 (2H, d, J=5.7 Hz), 4.59 (1H, t, J=8.9 Hz), 6.06 (1H, d, J=1.9 Hz), 6.11-6.18 (2H, m), 6.76 (2H, s), 6.89 (1H, d, J=8.0 Hz), 7.14 (1H, s), 7.27-7.32 (1H, m), 7.39 (1H, t, J=7.8 Hz), 7.47-7.52 (1H, m).

### Example 149

### [(3S)-6-{[3-(2-cyclopropyl-5-methoxy-1H-benzimidazol-1-yl)-2-methylbenzyl]amino}-2,3-dihydro-1-benzofuran-3-yl]acetic acid

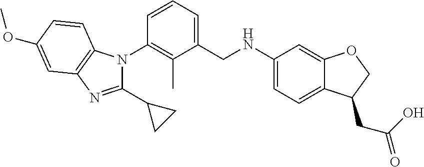

In the same manner as in Example 136, the title compound was obtained from methyl [(3S)-6-{[3-(2-cyclopropyl-5-methoxy-1H-benzimidazol-1-yl)-2-methylbenzyl]amino}-2,3-dihydro-1-benzofuran-3-yl]acetate.

¹H NMR (300 MHz, DMSO-d₆) δ 0.93-1.13 (4H, m), 1.52-1.63 (1H, m), 1.90 (3H, s), 2.38-2.47 (1H, m), 2.64 (1H, dd, J=16.4, 5.7 Hz), 3.53-3.66 (1H, m), 3.77 (3H, s), 4.09 (1H, dd, J=8.9, 6.7 Hz), 4.29 (2H, d, J=4.1 Hz), 4.60 (1H, t, J=8.9 Hz), 6.05 (1H, d, J=1.9 Hz), 6.14 (2H, dd, J=8.1, 1.9 Hz), 6.76 (2H, d, J=1.1 Hz), 6.92 (1H, d, J=8.1 Hz), 7.14 (1H, t, J=1.4 Hz), 7.27-7.33 (1H, m), 7.40 (1H, t, J=7.7 Hz), 7.46-7.52 (1H, m), 12.28 (1H, br s).

MS m/z 484 (M+H)⁺.

### Example 150

### methyl [(3S)-6-{[3-(2,5-dimethyl-1H-benzimidazol-1-yl)-2-methylbenzyl]amino}-2,3-dihydro-1-benzofuran-3-yl]acetate

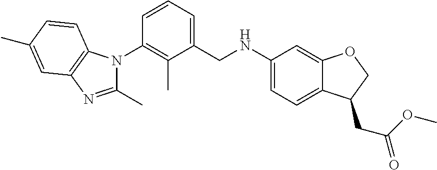

In the same manner as in Reference Example 114 and Example 134, the title compound was synthesized using methyl 3-[(2-amino-4-methylphenyl)amino]-2-methylbenzoate.

¹H NMR (300 MHz, DMSO-d₆) δ 1.83 (3H, s), 2.27 (3H, s), 2.41 (3H, s), 2.53-2.60 (1H, m), 2.65-2.76 (1H, m), 3.56-3.72 (4H, m), 4.11 (1H, dd, J=8.9, 6.6 Hz), 4.27 (2H, d, J=5.3 Hz), 4.59 (1H, t, J=8.9 Hz), 6.05 (1H, d, J=1.9 Hz), 6.14 (2H, dd, J=7.8, 2.1 Hz), 6.75 (1H, d, J=8.0 Hz), 6.89 (1H, d, J=8.3 Hz), 6.98 (1H, d, J=7.2 Hz), 7.24 (1H, d, J=6.4 Hz), 7.33-7.51 (3H, m).

### Example 151

### [(3S)-6-{[3-(2,5-dimethyl-1H-benzimidazol-1-yl)-2-methylbenzyl]amino}-2,3-dihydro-1-benzofuran-3-yl]acetic acid

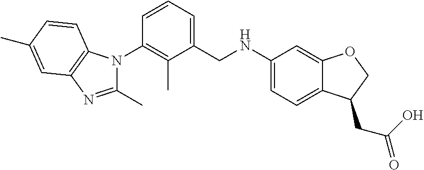

In the same manner as in Example 136, the title compound was synthesized using methyl [(3S)-6-{[3-(2,5-dimethyl-1H-benzimidazol-1-yl)-2-methylbenzyl]amino}-2,3-dihydro-1-benzofuran-3-yl]acetate.

¹H NMR (300 MHz, DMSO-d₆) δ 1.83 (3H, s), 2.28 (3H, s), 2.38-2.48 (4H, m), 2.58-2.69 (1H, m), 3.53-3.67 (1H, m), 4.10 (1H, dd, J=8.9, 6.7 Hz), 4.27 (2H, d, J=4.9 Hz), 4.60 (1H, t, J=8.9 Hz), 6.05 (1H, d, J=1.7 Hz), 6.10-6.19 (2H, m), 6.76 (1H, d, J=8.1 Hz), 6.92 (1H, d, J=8.1 Hz), 6.96-7.02 (1H, m), 7.25 (1H, d, J=7.0 Hz), 7.34-7.45 (2H, m), 7.47 (1H, d, J=7.0 Hz), 12.28 (1H, s).

MS m/z 442 (M+H)⁺.

### Example 152

### methyl [(3S)-6-{[3-(2,4-dimethyl-1H-benzimidazol-1-yl)-2-methylbenzyl]amino}-2,3-dihydro-1-benzofuran-3-yl]acetate

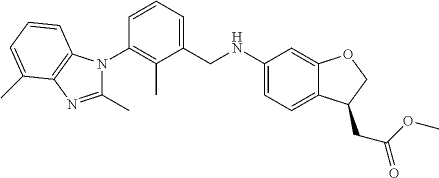

In the same manner as in Reference Example 114 and Example 134, the title compound was synthesized using methyl 3-[(2-amino-3-methylphenyl)amino]-2-methylbenzoate.

¹H NMR (300 MHz, DMSO-d₆) δ 1.84 (3H, s), 2.30 (3H, s), 2.54-2.60 (4H, m), 2.65-2.77 (1H, m), 3.57-3.67 (4H, m), 4.07-4.17 (1H, m), 4.28 (2H, d, J=5.7 Hz), 4.59 (1H, t, J=9.1 Hz), 6.06 (1H, s), 6.11-6.19 (2H, m), 6.65-6.71 (1H, m), 6.90 (1H, d, J=8.0 Hz), 6.99-7.08 (2H, m), 7.25 (1H, d, J=8.0 Hz), 7.38 (1H, t, J=7.8 Hz), 7.46-7.53 (1H, m).

### Example 153

### [(3S)-6-{[3-(2,4-dimethyl-1H-benzimidazol-1-yl)-2-methylbenzyl]amino}-2,3-dihydro-1-benzofuran-3-yl]acetic acid

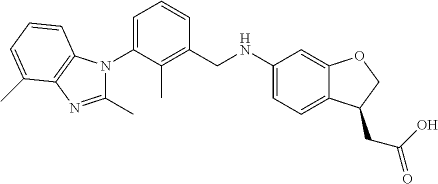

In the same manner as in Example 136, the title compound (yield 80%) was synthesized from methyl [(3S)-6-{[3-(2,4-dimethyl-1H-benzimidazol-1-yl-2-methyl-1H-benzimidazol-1-yl)-2-methylbenzyl]amino}-2,3-dihydro-1-benzofuran-3-yl]acetate.

¹H NMR (300 MHz, DMSO-d₆) δ 1.84 (3H, s), 2.32 (3H, s), 2.38-2.48 (1H, m), 2.57 (3H, s), 2.60-2.69 (1H, m), 3.54-3.65 (1H, m), 4.10 (1H, dd, J=8.9, 6.7 Hz), 4.29 (2H, br s), 4.60 (1H, t, J=8.9 Hz), 6.05 (1H, d, J=1.9 Hz), 6.10-6.18 (2H, m), 6.70 (1H, dd, J=7.0, 2.1 Hz), 6.92 (1H, d, J=8.1 Hz), 7.02-7.10 (2H, m), 7.26 (1H, d, J=6.8 Hz), 7.39 (1H, t, J=7.6 Hz), 7.47-7.52 (1H, m), 12.28 (1H, s).

MS m/z 442 (M+H)⁺.

### Example 154

### methyl [(3S)-6-{[2-methyl-3-(7-methyl-1H-benzimidazol-1-yl)benzyl]amino}-2,3-dihydro-1-benzofuran-3-yl]acetate

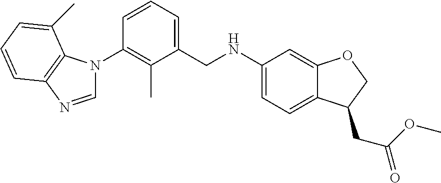

In the same manner as in Example 134, the title compound was synthesized from methyl 2-methyl-3-(7-methyl-1H-benzimidazol-1-yl)benzoate.

¹H NMR (300 MHz, DMSO-d₆) δ 1.84-1.89 (6H, m), 2.51-2.59 (1H, m), 2.65-2.75 (1H, m), 3.57-3.70 (4H, m), 4.10 (1H, dd, J=8.5, 6.6 Hz), 4.28 (2H, d, J=5.7 Hz), 4.59 (1H, t, J=8.9 Hz), 6.01 (1H, s), 6.11 (1H, d, J=8.1 Hz), 6.20 (1H, t, J=5.7 Hz), 6.88 (1H, d, J=8.1 Hz), 6.99 (1H, d, J=7.3 Hz), 7.15 (1H, t, J=7.6 Hz), 7.30-7.39 (2H, m), 7.48 (1H, dd, J=6.5, 2.5 Hz), 7.60 (1H, d, J=8.1 Hz), 8.17 (1H, s).

### Example 155

### [(3S)-6-{[2-methyl-3-(7-methyl-1H-benzimidazol-1-yl)benzyl]amino}-2,3-dihydro-1-benzofuran-3-yl]acetic acid

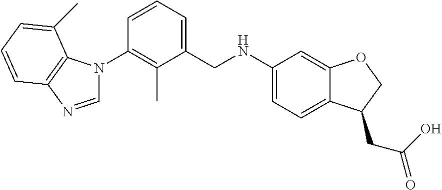

In the same manner as in Example 136, the title compound (yield 64%) was synthesized from methyl [(3S)-6-{[2-methyl-3-(7-methyl-1H-benzimidazol-1-yl)benzyl]amino}-2,3-dihydro-1-benzofuran-3-yl]acetate.

¹H NMR (300 MHz, DMSO-d₆) δ 1.84-1.88 (6H, m), 2.38-2.47 (1H, m), 2.59-2.68 (1H, m), 3.53-3.65 (1H, m), 4.05-4.13 (1H, m), 4.28 (2H, br s), 4.59 (1H, t, J=8.9 Hz), 6.00 (1H, s), 6.09-6.14 (1H, m), 6.16-6.21 (1H, m), 6.90 (1H, d, J=8.1 Hz), 7.00 (1H, d, J=7.3 Hz), 7.16 (1H, t, J=7.7 Hz), 7.32-7.39 (2H, m), 7.49 (1H, dd, J=6.6, 2.4 Hz), 7.60 (1H, d, J=7.9 Hz), 8.19 (1H, s), 12.27 (1H, s).

MS m/z 428 (M+H)⁺.

### Example 156

### methyl [(3S)-6-{[3-(2-ethyl-1H-benzimidazol-1-yl)-2-methylbenzyl]amino}-2,3-dihydro-1-benzofuran-3-yl]acetate

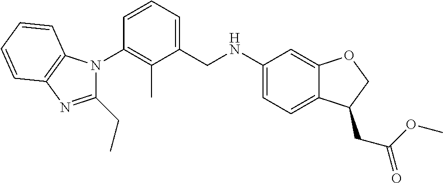

In the same manner as in Example 134, the title compound was synthesized from methyl 3-(2-ethyl-1H-benzimidazol-1-yl)-2-methylbenzoate.

¹H NMR (300 MHz, DMSO-d₆) δ 1.19-1.27 (3H, m), 1.82 (3H, s), 2.53-2.63 (3H, m), 2.66-2.76 (1H, m), 3.57-3.68 (4H, m), 4.11 (1H, dd, J=8.9, 6.6 Hz), 4.28 (2H, d, J=5.7 Hz), 4.59 (1H, t, J=8.9 Hz), 6.05 (1H, d, J=1.9 Hz), 6.12-6.18 (2H, m), 6.88 (2H, t, J=8.0 Hz), 7.11-7.30 (3H, m), 7.39 (1H, t, J=7.8 Hz), 7.46-7.53 (1H, m), 7.66 (1H, d, J=7.2 Hz).

### Example 157

### [(3S)-6-{[3-(2-ethyl-1H-benzimidazol-1-yl)-2-methylbenzyl]amino}-2,3-dihydro-1-benzofuran-3-yl]acetic acid

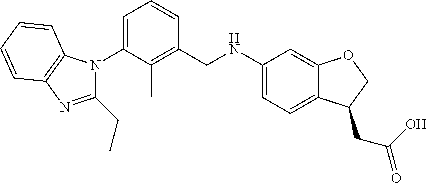

In the same manner as in Example 136, the title compound was synthesized from methyl [(3S)-6-{[3-(2-ethyl-1H-benzimidazol-1-yl)-2-methylbenzyl]amino}-2,3-dihydro-1-benzofuran-3-yl]acetate.

¹H NMR (300 MHz, DMSO-d₆) δ 1.22 (3H, t, J=7.5 Hz), 1.82 (3H, s), 2.42 (1H, dd, J=16.6, 9.0 Hz), 2.53-2.68 (3H, m), 3.53-3.65 (1H, m), 4.09 (1H, dd, J=8.9, 6.6 Hz), 4.28 (2H, d, J=5.7 Hz), 4.60 (1H, t, J=8.9 Hz), 6.05 (1H, d, J=1.9 Hz), 6.10-6.18 (2H, m), 6.90 (2H, dd, J=15.4, 7.5 Hz), 7.11-7.30 (3H, m), 7.39 (1H, t, J=7.5 Hz), 7.47-7.52 (1H, m), 7.67 (1H, d, J=7.2 Hz), 12.34 (1H, br. s).

MS m/z 442 (M+H)⁺.

### Example 158

### sodium [(3S)-6-{[3-(2-ethyl-1H-benzimidazol-1-yl)-2-methylbenzyl]amino}-2,3-dihydro-1-benzofuran-3-yl]acetate

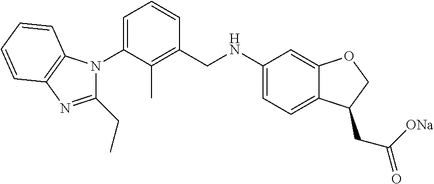

In the same manner as in Example 137, the title compound was synthesized from [(3S)-6-{[3-(2-ethyl-1H-benzimidazol-1-yl)-2-methylbenzyl]amino}-2,3-dihydro-1-benzofuran-3-yl]acetic acid.

¹H NMR (300 MHz, DMSO-d₆) δ 1.22 (3H, t, J=7.5 Hz), 1.82 (3H, s), 1.96 (1H, dd, J=14.9, 10.0 Hz), 2.30 (1H, dd, J=15.1, 4.9 Hz), 2.54-2.63 (2H, m), 3.46-3.57 (1H, m), 4.05 (1H, dd, J=8.7, 7.2 Hz), 4.27 (2H, d, J=5.7 Hz), 4.58 (1H, t, J=8.9 Hz), 5.98-6.13 (3H, m), 6.84-6.92 (2H, m), 7.12-7.29 (3H, m), 7.38 (1H, t, J=7.7 Hz), 7.51 (1H, d, J=7.2 Hz), 7.67 (1H, d, J=7.2 Hz).

### Example 159

### methyl [(3S)-6-{[3-(2,6-dimethyl-1H-benzimidazol-1-yl)-2-methylbenzyl]amino}-2,3-dihydro-1-benzofuran-3-yl]acetate

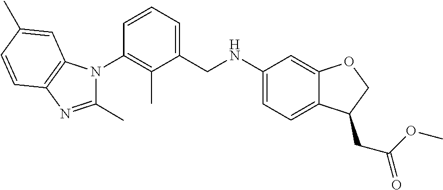

In the same manner as in Example 134, the title compound was synthesized from methyl 3-(2,6-dimethyl-1H-benzimidazol-1-yl)-2-methylbenzoate.

¹H NMR (300 MHz, DMSO-d₆) δ 1.84 (3H, s), 2.26 (3H, s), 2.34 (3H, s), 2.53-2.59 (1H, m), 2.66-2.75 (1H, m), 3.60-3.66 (4H, m), 4.11 (1H, dd, J=8.7, 6.4 Hz), 4.28 (2H, d, J=5.7 Hz), 4.59 (1H, t, J=8.9 Hz), 6.06 (1H, d, J=1.9 Hz); 6.15 (2H, dd, J=7.8, 2.1 Hz), 6.66 (1H, s), 6.90 (1H, d, J=8.0 Hz), 7.02 (1H, d, J=8.0 Hz), 7.24 (1H, d, J=6.8 Hz), 7.38 (1H, t, J=7.6 Hz), 7.46-7.53 (2H, m).

### Example 160

### [(3S)-6-{[3-(2,6-dimethyl-1H-benzimidazol-1-yl)-2-methylbenzyl]amino}-2,3-dihydro-1-benzofuran-3-yl]acetic acid

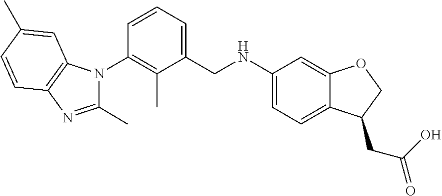

In the same manner as in Example 136, the title compound (yield 86%) was synthesized from methyl [(3S)-6-{[3-(2,6-dimethyl-1H-benzimidazol-1-yl)-2-methylbenzyl]amino}-2,3-dihydro-1-benzofuran-3-yl]acetate.

¹H NMR (300 MHz, DMSO-d₆) δ 1.84 (3H, s), 2.26 (3H, s), 2.32-2.46 (4H, m), 2.62 (1H, dd, J=16.4, 5.5 Hz), 3.54-3.65 (1H, m), 4.09 (1H, dd, J=9.0, 6.8 Hz), 4.28 (2H, d, J=4.5 Hz), 4.60 (1H, t, J=9.0 Hz), 6.05 (1H, d, J=1.9 Hz), 6.09-6.19 (2H, m), 6.66 (1H, s), 6.92 (1H, d, J=8.3 Hz), 7.00-7.05 (1H, m), 7.24 (1H, d, J=7.5 Hz), 7.38 (1H, t, J=7.5 Hz), 7.46-7.54 (2H, m).

MS m/z 442 (M+H)⁺.

### Example 161

### methyl [(3S)-6-{[3-(6-methoxy-2-methyl-1H-benzimidazol-1-yl)-2-methylbenzyl]amino}-2,3-dihydro-1-benzofuran-3-yl]acetate

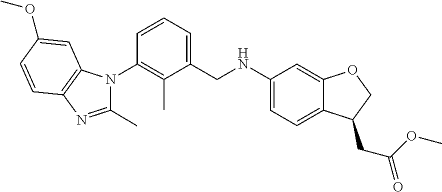

In the same manner as in Example 134, the title compound was synthesized from methyl 3-(6-methoxy-2-methyl-1H-benzimidazol-1-yl)-2-methylbenzoate.

¹H NMR (300 MHz, DMSO-d₆) δ 1.85 (3H, s), 2.24 (3H, s), 2.52-2.58 (1H, m), 2.66-2.75 (1H, m), 3.60-3.64 (4H, m), 3.67 (3H, s), 4.11 (1H, dd, J=9.1, 6.4 Hz), 4.29 (2H, d, J=5.3 Hz), 4.59 (1H, t, J=9.1 Hz), 6.05 (1H, d, J=1.9 Hz), 6.15 (2H, dd, J=7.8, 2.1 Hz), 6.31 (1H, d, J=2.7 Hz), 6.83 (1H, dd, J=8.7, 2.3 Hz), 6.89 (1H, d, J=8.3 Hz), 7.26 (1H, d, J=7.6 Hz), 7.39 (1H, t, J=7.8 Hz), 7.47-7.54 (2H, m).

### Example 162

### [(3S)-6-{[3-(6-methoxy-2-methyl-1H-benzimidazol-1-yl)-2-methylbenzyl]amino}-2,3-dihydro-1-benzofuran-3-yl]acetic acid

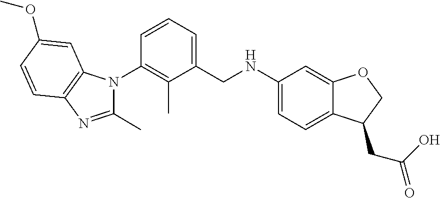

In the same manner as in Example 136, the title compound (yield 52%) was synthesized using methyl [(3S)-6-{[3-(6-methoxy-2-methyl-1H-benzimidazol-1-yl)-2-methylbenzyl]amino}-2,3-dihydro-1-benzofuran-3-yl]acetate.

¹H NMR (300 MHz, DMSO-d₆) δ 1.85 (3H, s), 2.24 (3H, s), 2.37-2.47 (1H, m), 2.58-2.68 (1H, m), 3.54-3.63 (1H, m), 3.67 (3H, s), 4.09 (1H, dd, J=8.9, 6.6 Hz), 4.29 (2H, d, J=5.3 Hz), 4.59 (1H, t, J=8.9 Hz), 6.05 (1H, d, J=1.9 Hz), 6.11-6.18 (2H, m), 6.32 (1H, d, J=2.3 Hz), 6.83 (1H, dd, J=8.7, 2.3 Hz), 6.92 (1H, d, J=7.9 Hz), 7.24-7.28 (1H, m), 7.39 (1H, t, J=7.7 Hz), 7.47-7.54 (2H, m).

MS m/z 458 (M+H)⁺.

### Example 163

### methyl [(3S)-6-({3-[2-(methoxymethyl)-1H-benzimidazol-1-yl]-2-methylbenzyl}amino)-2,3-dihydro-1-benzofuran-3-yl]acetate

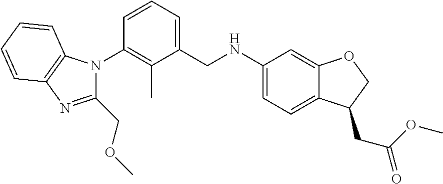

In the same manner as in Example 134, the title compound was synthesized using methyl 3-[2-(methoxymethyl)-1H-benzimidazol-1-yl]-2-methylbenzoate.

¹H NMR (300 MHz, DMSO-d₆) δ 1.83 (3H, d, J=3.8 Hz), 2.52-2.60 (1H, m), 2.66-2.76 (1H, m), 3.16 (3H, s), 3.56-3.69 (4H, m), 4.11 (1H, dd, J=8.9, 6.6 Hz), 4.28 (2H, d, J=5.3 Hz), 4.59 (1H, t, J=8.9 Hz), 6.03-6.07 (1H, m), 6.11-6.20 (2H, m), 6.85-6.97 (2H, m), 7.12-7.30 (3H, m), 7.33-7.42 (1H, m), 7.46-7.51 (1H, m), 7.61-7.79 (1H; m).

### Example 164

### [(3S)-6-({3-[2-(methoxymethyl)-1H-benzimidazol-1-yl]-2-methylbenzyl}amino)-2,3-dihydro-1-benzofuran-3-yl]acetic acid

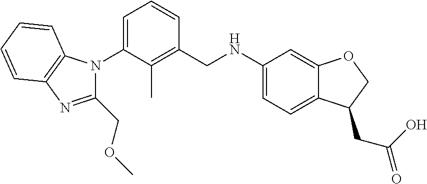

In the same manner as in Example 136, the title compound was synthesized using methyl [(3S)-6-({3-[2-(methoxymethyl)-1H-benzimidazol-1-yl]-2-methylbenzyl}amino)-2,3-dihydro-1-benzofuran-3-yl]acetate.

¹H NMR (300 MHz, DMSO-d₆) δ 1.82 (3H, s), 2.38-2.47 (1H, m), 2.64 (1H, dd, J=16.4, 5.5 Hz), 3.16 (3H, s), 3.53-3.65 (1H, m), 4.09 (1H, dd, J=9.0, 6.8 Hz), 4.28 (2H, d, J=4.1 Hz), 4.35-4.51 (2H, m), 4.60 (1H, t, J=8.9 Hz), 6.04 (1H, s), 6.11-6.17 (2H, m), 6.89-6.97 (2H, m), 7.22-7.32 (3H, m), 7.37 (1H, t, J=7.7 Hz), 7.49 (1H, d, J=6.4 Hz), 7.72-7.78 (1H, m), 12.27 (1H, s).

MS m/z 458 (M+H)⁺.

### Example 165

### methyl [(3S)-6-({2-methyl-3-[2-(methylsulfanyl)-1H-benzimidazol-1-yl]benzyl}amino)-2,3-dihydro-1-benzofuran-3-yl]acetate

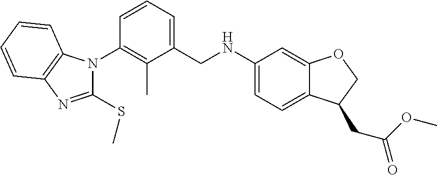

In the same manner as in Example 134, the title compound was synthesized using methyl 2-methyl-3-[2-(methylsulfanyl)-1H-benzimidazol-1-yl]benzoate.

¹H NMR (300 MHz, DMSO-d₆) δ 1.86 (3H, s), 2.53-2.59 (1H, m), 2.63-2.75 (4H, m), 3.57-3.68 (4H, m), 4.08-4.14 (1H, m), 4.28 (2H, d, J=5.7 Hz), 6.05 (1H, d, J=1.5 Hz), 6.11-6.19 (2H, m), 6.88 (2H, t, J=7.6 Hz), 7.11-7.28 (3H, m), 7.38 (1H, t, J=7.8 Hz), 7.48-7.53 (1H, m), 7.65 (1H, d, J=7.6 Hz).

### Example 166

### [(3S)-6-({2-methyl-3-[2-(methylsulfanyl)-1H-benzimidazol-1-yl]benzyl}amino)-2,3-dihydro-1-benzofuran-3-yl]acetic acid

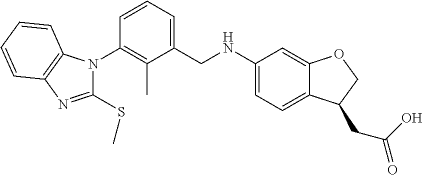

In the same manner as in Example 136, the title compound (yield 53%) was synthesized from methyl [(3S)-6-({2-methyl-3-[2-(methylsulfanyl)-1H-benzimidazol-1-yl]benzyl}amino)-2,3-dihydro-1-benzofuran-3-yl]acetate.

¹H NMR (300 MHz, DMSO-d₆) δ 1.87 (3H, s), 2.28-2.40 (2H, m), 2.58 (1H, dd, J=16.1, 5.5 Hz), 2.69 (3H, s), 3.50-3.66 (1H, m), 4.08 (1H, dd, J=8.7, 6.8 Hz), 4.28 (2H, d, J=4.5 Hz), 4.59 (1H, t, J=8.9 Hz), 6.04 (1H, d, J=1.9 Hz), 6.08-6.18 (2H, m), 6.89 (2H, dd, J=13.1, 7.8 Hz), 7.10-7.29 (3H, m), 7.38 (1H, t, J=7.8 Hz), 7.51 (1H, d, J=7.2 Hz), 7.65 (1H, d, J=7.6 Hz).

MS m/z 460 (M+H)⁺.

### Example 167

### methyl {(3S)-6-[(3-{2-[(4-chlorobenzyl)sulfanyl]-1H-benzimidazol-1-yl}-2-methylbenzyl)amino]-2,3-dihydro-1-benzofuran-3-yl}acetate

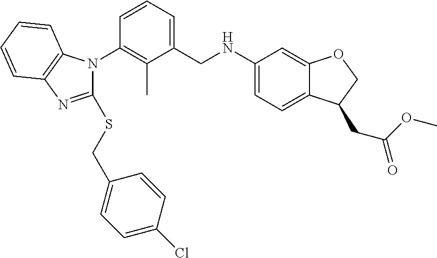

In the same manner as in Example 134, the title compound was synthesized using methyl 3-{2-[(4-chlorobenzyl)sulfanyl]-1H-benzimidazol-1-yl}-2-methylbenzoate.

¹H NMR (300 MHz, DMSO-d₆) δ 1.79 (3H, s), 2.52-2.59 (1H, m), 2.65-2.75 (1H, m), 3.57-3.66 (4H, m), 4.10 (1H, dd, J=8.7, 6.4 Hz), 4.25 (2H, d, J=5.7 Hz), 4.53-4.64 (3H, m), 6.03 (1H, d, J=1.9 Hz), 6.09-6.18 (2H, m), 6.88 (2H, d, J=8.0 Hz), 7.12-7.51 (9H, m).

### Example 168

### sodium {(3S)-6-[(3-{2-[(4-chlorobenzyl)sulfanyl]-1H-benzimidazol-1-yl}-2-methylbenzyl)amino]-2,3-dihydro-1-benzofuran-3-yl}acetate

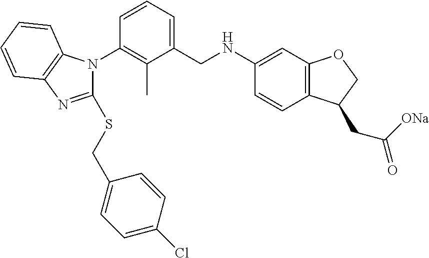

Methyl {(3S)-6-[(3-{2-[(4-chlorobenzyl)sulfanyl]-1H-benzimidazol-1-yl}-2-methylbenzyl)amino]-2,3-dihydro-1-benzofuran-3-yl}acetate (120 mg, 0.21 mmol) was dissolved in tetrahydrofuran (5 mL) and methanol (5 mL), 8N aqueous sodium hydroxide solution (0.5 mL) was added, and the mixture was stirred at 40° C. for 2 hr. Water (10 mL) was added, and the volatile component was evaporated under reduced pressure. The resulting precipitate was collected to give the title compound (140 mg) as a white solid.

¹H NMR (300 MHz, DMSO-d₆) δ 1.79 (3H, s), 1.87-2.00 (1H, m), 2.23-2.31 (1H, m), 3.30 (2H, s), 3.49 (1H, br s), 3.99-4.07 (1H, m), 4.24 (2H, d, J=4.9 Hz), 4.58-4.60 (1H, m), 5.95-6.02 (2H, m), 6.07 (1H, d, J=8.3 Hz), 6.84-6.91 (2H, m), 7.11-7.28 (3H, m), 7.31-7.53 (6H, m), 7.68 (1H, d, J=7.6 Hz).

### Example 169

### methyl {(3S)-6-[(2-methyl-3-{5-methyl-2-[(2R)-tetrahydrofuran-2-yl]-1H-benzimidazol-1-yl}benzyl)amino]-2,3-dihydro-1-benzofuran-3-yl}acetate

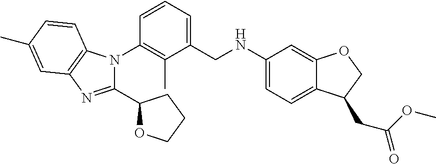

In the same manner as in Example 145, the title compound was synthesized.

¹H NMR (300 MHz, DMSO-d₆) δ 1.76-1.90 (4H, m), 2.00-2.20 (2H, m), 2.39-2.45 (4H, m), 2.53-2.61 (1H, m), 2.65-2.76 (1H, m), 3.56-3.74 (6H, m), 4.11 (1H, dd, J=8.9, 6.6 Hz), 4.26 (2H, d, J=3.4 Hz), 4.59 (1H, t, J=8.9 Hz), 4.68-4.85 (1H, m), 6.04 (1H, s), 6.10-6.20 (2H, m), 6.78 (1H, dd, J=8.1, 4.7 Hz), 6.89 (1H, d, J=8.0 Hz), 7.04 (1H, d, J=8.3 Hz), 7.20-7.29 (1H, m), 7.31-7.40 (1H, m), 7.43-7.54 (2H, m).

### Example 170

### {(3S)-6-[(2-methyl-3-{5-methyl-2-[(2R)-tetrahydrofuran-2-yl]-1H-benzimidazol-1-yl}benzyl)amino]-2,3-dihydro-1-benzofuran-3-yl}acetic acid

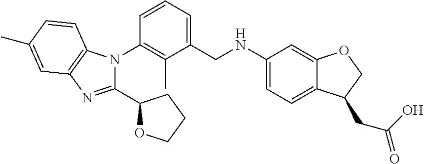

In the same manner as in Example 136, the title compound (yield 87%) was synthesized using methyl {(3S)-6-[(2-methyl-3-{5-methyl-2-[(2R)-tetrahydrofuran-2-yl]-1H-benzimidazol-1-yl}benzyl)amino]-2,3-dihydro-1-benzofuran-3-yl}acetate.

¹H NMR (300 MHz, DMSO-d₆) δ 1.70-2.20 (6H, m), 2.35-2.47 (4H, m), 2.63 (1H, dd, J=16.6, 5.7 Hz), 3.51-3.76 (4H, m), 4.09 (1H, dd, J=9.0, 6.8 Hz), 4.27 (2H, d, J=3.4 Hz), 4.60 (1H, t, J=9.0 Hz), 4.68-4.86 (1H, m), 6.04 (1H, d, J=1.9 Hz), 6.10-6.18 (2H, m), 6.78 (1H, dd, J=8.3, 4.9 Hz), 6.92 (1H, d, J=8.3 Hz), 7.04 (1H, d, J=8.3 Hz), 7.21-7.29 (1H, m), 7.31-7.40 (1H, m), 7.44-7.54 (2H, m).

MS m/z 498 (M+H)⁺.

### Example 171

### methyl [(3S)-6-{[3-(6-chloro-2-ethyl-1H-benzimidazol-1-yl)-2-methylbenzyl]amino}-2,3-dihydro-1-benzofuran-3-yl]acetate

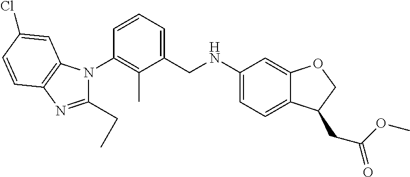

In the same manner as in Example 134, the title compound was synthesized using methyl 3-(6-chloro-2-ethyl-1H-benzimidazol-1-yl)-2-methylbenzoate.

¹H NMR (300 MHz, DMSO-d₆) δ 1.19-1.25 (3H, m), 1.83 (3H, s), 2.53-2.63 (3H, m), 2.66-2.76 (1H, m), 3.57-3.69 (4H, m), 4.08-4.15 (1H, m), 4.28 (2H, d, J=5.7 Hz), 4.59 (1H, t, J=8.9 Hz), 6.06 (1H, d, J=1.9 Hz), 6.11-6.18 (2H, m), 6.87-6.93 (2H, m), 7.21-7.32 (2H, m), 7.39 (1H, t, J=7.8 Hz), 7.48-7.54 (1H, m), 7.69 (1H, d, J=8.7 Hz).

### Example 172

### [(3S)-6-{[3-(6-chloro-2-ethyl-1H-benzimidazol-1-yl)-2-methylbenzyl]amino}-2,3-dihydro-1-benzofuran-3-yl]acetic acid

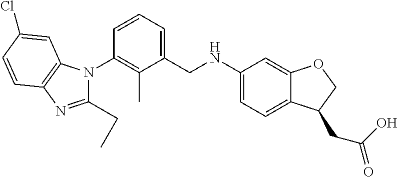

Methyl [(3S)-6-{[3-(6-chloro-2-ethyl-1H-benzimidazol-1-yl)-2-methylbenzyl]amino}-2,3-dihydro-1-benzofuran-3-yl]acetate (310 mg, 0.63 mmol) was dissolved in tetrahydrofuran (4 mL) and methanol (4 mL), 1N aqueous sodium hydroxide solution (1.9 mL) was added, and the mixture was stirred at room temperature for 4 hr. The volatile component was evaporated under reduced pressure, and 1 M aqueous hydrochloric acid solution (1.9 mL) was added. The resulting precipitate was collected to give the title compound (230 mg, yield 77%) as a white solid.

¹H NMR (300 MHz, DMSO-d₆) δ 1.18-1.27 (3H, m), 1.83 (3H, s), 2.30 (1H, s), 2.37-2.48 (1H, m), 2.53-2.71 (2H, m), 3.53-3.66 (1H, m), 4.10 (1H, dd, J=9.0, 6.8 Hz), 4.28 (2H, d, J=4.9 Hz), 4.60 (1H, t, J=9.0 Hz), 6.06 (1H, d, J=1.9 Hz), 6.09-6.19 (2H, m), 6.87-6.96 (2H, m), 7.11-7.19 (1H, m), 7.22-7.32 (2H, m), 7.39 (1H, t, J=7.7 Hz), 7.48-7.54 (1H, m), 7.69 (1H, d, J=8.7 Hz).

MS m/z 476 (M+H)⁺.

### Example 173

### sodium [(3S)-6-{[3-(6-chloro-2-ethyl-1H-benzimidazol-1-yl)-2-methylbenzyl]amino}-2,3-dihydro-1-benzofuran-3-yl]acetate

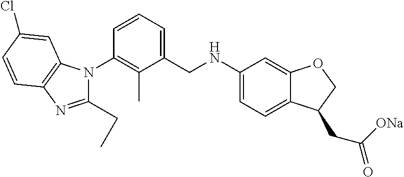

[(3S)-6-{[3-(6-Chloro-2-ethyl-1H-benzimidazol-1-yl)-2-methylbenzyl]amino}-2,3-dihydro-1-benzofuran-3-yl]acetic acid (95 mg, 0.2 mmol) was mixed with acetonitrile (5 mL), and 1 M aqueous sodium hydroxide solution (0.2 mL) was added. The volatile component was evaporated under reduced pressure and the resulting precipitate was collected to give the title compound (95 mg, yield 96%) as a white solid.

¹H NMR (300 MHz, DMSO-d₆) δ 1.17-1.26 (3H, m), 1.83 (3H, s), 1.97 (1H, dd, J=15.1, 9.8 Hz), 2.31 (1H, dd, J=15.1, 4.9 Hz), 2.52-2.61 (1H, m), 3.53 (1H, br s), 4.00-4.10 (1H, m), 4.27 (2H, d, J=5.3 Hz), 4.58 (1H, t, J=8.9 Hz), 5.97-6.04 (2H, m), 6.10 (1H, dd, J=8.1, 1.7 Hz), 6.84-6.94 (2H, m), 7.20-7.32 (2H, m), 7.39 (1H, t, J=7.7 Hz), 7.52 (1H, d, J=7.2 Hz), 7.69 (1H, d, J=8.7 Hz).

MS m/z 476 (M+H)⁺ (as free form).

### Example 174

### methyl [(3S)-6-{[3-(2-ethyl-5-methoxy-1H-benzimidazol-1-yl)-2-methylbenzyl]amino}-2,3-dihydro-1-benzofuran-3-yl]acetate

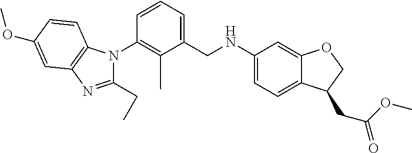

In the same manner as in Reference Example 122 and Example 134, the title compound was synthesized from methyl 3-[(2-amino-4-methoxyphenyl)amino]-2-methylbenzoate.

¹H NMR (300 MHz, DMSO-d₆) δ 1.18-1.24 (3H, m), 1.82 (3H, s), 2.52-2.61 (3H, m), 2.66-2.75 (1H, m), 3.58-3.66 (4H, m), 3.79 (3H, s), 4.11 (1H, dd, J=8.9, 6.6 Hz), 4.27 (2H, d, J=5.7 Hz), 6.05 (1H, d, J=1.9 Hz), 6.11-6.18 (2H, m), 6.73-6.82 (2H, m), 6.89 (1H, d, J=8.0 Hz), 7.19-7.27 (2H, m), 7.37 (1H, t, J=7.8 Hz), 7.45-7.51 (1H, m).

### Example 175

### [(3S)-6-{[3-(2-ethyl-5-methoxy-1H-benzimidazol-1-yl)-2-methylbenzyl]amino}-2,3-dihydro-1-benzofuran-3-yl]acetic acid

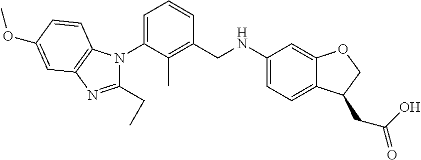

In the same manner as in Example 172, the title compound (yield 90%) was synthesized using methyl [(3S)-6-{[3-(2-ethyl-5-methoxy-1H-benzimidazol-1-yl)-2-methylbenzyl]amino}-2,3-dihydro-1-benzofuran-3-yl]acetate.

¹H NMR (300 MHz, DMSO-d₆) δ 1.20 (3H, t, J=7.6 Hz), 1.82 (3H, s), 2.36-2.46 (1H, m), 2.52-2.69 (3H, m), 3.54-3.65 (1H, m), 3.79 (3H, s), 4.09 (1H, dd, J=8.9, 6.6 Hz), 4.27 (2H, d, J=4.9 Hz), 4.60 (1H, t, J=9.1 Hz), 6.04 (1H, s), 6.08-6.17 (2H, m), 6.72-6.82 (2H, m), 6.92 (1H, d, J=8.0 Hz), 7.18-7.29 (2H, m), 7.37 (1H, t, J=7.6 Hz), 7.45-7.51 (1H, m), 12.27 (1H, br s).

MS m/z 472 (M+H)⁺.

### Example 176

### methyl [(3S)-6-{[3-(2-ethoxy-6-methoxy-1H-benzimidazol-1-yl)-2-methylbenzyl]amino}-2,3-dihydro-1-benzofuran-3-yl]acetate

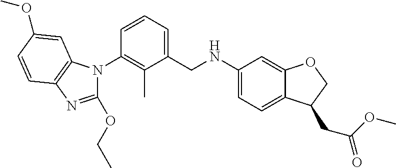

In the same manner as in Reference Example 108 and Example 134, the title compound was synthesized using methyl 3-[(2-amino-5-methoxyphenyl)amino]-2-methylbenzoate.

¹H NMR (300 MHz, DMSO-d₆) δ 1.31 (3H, t, J=7.0 Hz), 1.94 (3H, s), 2.52-2.59 (1H, m), 2.66-2.75 (1H, m), 3.57-3.69 (7H, m), 4.10 (1H, dd, J=9.0, 6.4 Hz), 4.27 (2H, d, J=5.7 Hz), 4.43-4.64 (3H, m), 6.05 (1H, d, J=1.9 Hz), 6.11-6.18 (2H, m), 6.31 (1H, d, J=2.6 Hz), 6.76 (1H, dd, J=8.7, 2.6 Hz), 6.89 (1H, d, J=7.9 Hz), 7.21-7.27 (1H, m), 7.31-7.47 (3H, m).

MS m/z 502 (M+H)⁺.

### Example 177

### [(3S)-6-{[3-(2-ethoxy-6-methoxy-1H-benzimidazol-1-yl)-2-methylbenzyl]amino}-2,3-dihydro-1-benzofuran-3-yl]acetic acid

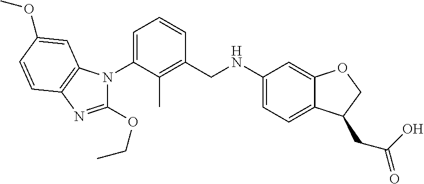

In the same manner as in Example 172, the title compound (yield 78%) was synthesized using methyl [(3S)-6-{[3-(2-ethoxy-6-methoxy-1H-benzimidazol-1-yl)-2-methylbenzyl]amino}-2,3-dihydro-1-benzofuran-3-yl]acetate.

¹H NMR (300 MHz, DMSO-d₆) δ 1.31 (3H, t, J=7.0 Hz), 1.95 (3H, s), 2.38 (1H, dd, J=16.3, 9.1 Hz), 2.60 (1H, dd, J=16.3, 5.7 Hz), 3.53-3.64 (1H, m), 3.67 (3H, s), 4.08 (1H, dd, J=8.7, 6.8 Hz), 4.27 (2H, d, J=5.3 Hz), 4.43-4.64 (3H, m), 6.03-6.17 (3H, m), 6.31 (1H, d, J=2.3 Hz), 6.76 (1H, dd, J=8.7, 2.7 Hz), 6.91 (1H, d, J=8.0 Hz), 7.21-7.27 (1H, m), 7.31-7.48 (3H, m).

MS m/z 488 (M+H)⁺.

### Example 178

### methyl [(3S)-6-{[3-(2-ethyl-6-fluoro-1H-benzimidazol-1-yl)-2-methylbenzyl]amino}-2,3-dihydro-1-benzofuran-3-yl]acetate

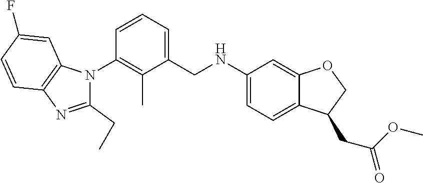

To a solution of 3-(2-ethyl-6-fluoro-1H-benzimidazol-1-yl)-2-methylbenzaldehyde (5.5 g, 19.48 mmol), methyl [(3S)-6-amino-2,3-dihydro-1-benzofuran-3-yl]acetate (4.4 g, 21.43 mmol) and acetic acid (4 mL) in acetonitrile (200 mL) was added sodium triacetoxyborohydride (12.3 g, 58.44 mmol), and the mixture was stirred at room temperature for 2 hr. The mixture was diluted with aqueous sodium bicarbonate solution and extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=70:30-10:90) to give the title compound (7.1 g, yield 77%).

¹H NMR (300 MHz, CDCl₃) δ 1.34 (3H, t, J=7.5 Hz), 1.92 (3H, s), 2.47-2.69 (3H, m), 2.75 (1H, dd, J=16.6, 5.3 Hz), 3.67-3.86 (4H, m), 4.03 (1H, br s), 4.24 (1H, dd, J=9.4, 6.0 Hz), 4.35 (2H, s), 4.73 (1H, t, J=8.9 Hz), 6.11-6.24 (2H, m), 6.58 (1H, dd, J=8.5, 2.4 Hz), 6.94-7.06 (2H, m), 7.17 (1H, d, J=7.5 Hz), 7.36 (1H, t, J=7.7 Hz), 7.55 (1H, d, J=7.5 Hz), 7.71 (1H, dd, J=8.9, 4.7 Hz).

MS m/z 474 (M+H)⁺.

### Example 179

### [(3S)-6-{[3-(2-ethyl-6-fluoro-1H-benzimidazol-1-yl)-2-methylbenzyl]amino}-2,3-dihydro-1-benzofuran-3-yl]acetic acid

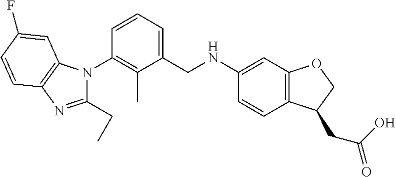

Methyl [(3S)-6-{[3-(2-ethyl-6-fluoro-1H-benzimidazol-1-yl)-2-methylbenzyl]amino}-2,3-dihydro-1-benzofuran-3-yl]acetate (1400 mg) was subjected to chiral resolution (axial asymmetric resolution) by supercritical fluid chromatography to give tR1 (640 mg, >99.9% d.e.) and tR2 (640 mg, 99.5% d.e.). The obtained compounds were each subjected to alkali hydrolysis in the same manner as in Example 172 to give the title compounds.

(high performance liquid chromatography conditions)
column: CHIRALPAK IC (manufactured by Daicel Chemical Industries, Ltd.)
mobile phase: carbon dioxide/methanol (volume ratio: 65/35)
flow rate: 50 mL/min
detection: UV (220 nm)
temperature: 35° C.
retention time: tR1 4.5 min, tR2 5.7 min

### [(3S)-6-{[3-(2-ethyl-6-fluoro-1H-benzimidazol-1-yl)-2-methylbenzyl]amino}-2,3-dihydro-1-benzofuran-3-yl]acetic acid (tR1)

¹H NMR (300 MHz, DMSO-d₆) δ 1.21 (3H, t, J=7.4 Hz), 1.84 (3H, s), 2.38-2.48 (1H, m), 2.52-2.68 (3H, m), 3.53-3.67 (1H, m), 4.10 (1H, dd, J=8.9, 6.6 Hz), 4.28 (2H, d, J=5.3 Hz), 4.60 (1H, t, J=8.9 Hz), 6.02-6.19 (3H, m), 6.69 (1H, dd, J=8.9, 2.5 Hz), 6.92 (1H, d, J=8.3 Hz), 7.01-7.11 (1H, m), 7.26-7.31 (1H, m), 7.39 (1H, t, J=7.6 Hz), 7.47-7.52 (1H, m), 7.67 (1H, dd, J=8.7, 4.9 Hz), 12.27 (1H, br s).

MS m/z 460 (M+H)⁺.

### [(3S)-6-{[3-(2-ethyl-6-fluoro-1H-benzimidazol-1-yl)-2-methylbenzyl]amino}-2,3-dihydro-1-benzofuran-3-yl]acetic acid (tR2)

¹H NMR (300 MHz, DMSO-d₆) δ 1.21 (3H, t, J=7.4 Hz), 1.84 (3H, s), 2.36-2.46 (1H, m), 2.52-2.67 (3H, m), 3.54-3.65 (1H, m), 4.09 (1H, dd, J=8.9, 6.6 Hz), 4.28 (2H, d, J=4.9 Hz), 4.60 (1H, t, J=8.9 Hz), 6.04-6.17 (3H, m), 6.69 (1H, dd, J=8.9, 2.5 Hz), 6.92 (1H, d, J=8.0 Hz), 7.02-7.11 (1H, m), 7.26-7.31 (1H, m), 7.39 (1H, t, J=7.8 Hz), 7.48-7.52 (1H, m), 7.67 (1H, dd, J=8.7, 4.9 Hz).

MS m/z 460 (M+H)⁺.

### Example 180

### [(3S)-6-({3-[2-ethoxy-4-(2,2,2-trifluoroethoxy)-1H-benzimidazol-1-yl]-2-methylbenzyl}amino)-2,3-dihydro-1-benzofuran-3-yl]acetic acid

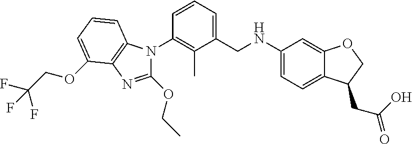

Methyl [(3S)-6-{[3-(2-ethoxy-4-hydroxy-1H-benzimidazol-1-yl)-2-methylbenzyl](trifluoroacetyl)amino}-2,3-dihydro-1-benzofuran-3-yl]acetate (116 mg, 0.2 mmol), 2,2,2-trifluoroethyl methanesulfonate (53 mg, 0.3 mmol) and potassium carbonate (55 mg, 0.4 mmol) were suspended in N,N-dimethylformamide (3 mL) and the suspension was stirred at 60° C. for 2 hr. The reaction mixture was allowed to cool, diluted with saturated brine, and the mixture was extracted with ethyl acetate. The extract was dried over anhydrous magnesium sulfate, and the volatile component was evaporated under reduced pressure. The residue was purified by silica gel column chromatography (hexane:ethyl acetate=90:10-40:60) to give an oil (120 mg). The obtained oil was dissolved in methanol (5 mL)-tetrahydrofuran (5 mL), 1 M aqueous sodium hydroxide solution (0.5 mL) was added, and the mixture was stirred at 50° C. for 1 hr. Water (10 mL) was added, the volatile component was evaporated under reduced pressure, and 1 M aqueous hydrochloric acid solution (0.5 mL) was added. The resulting precipitate was collected to give the title compound (55 mg, yield 50%) as a white solid.

¹H NMR (300 MHz, DMSO-d₆) δ 1.33 (3H, t, J=7.2 Hz), 1.93 (3H, s), 2.37-2.47 (1H, m), 2.63 (1H, dd, J=16.4, 5.5 Hz), 3.53-3.67 (1H, m), 4.09 (1H, dd, J=8.9, 6.6 Hz), 4.27 (2H, d, J=5.3 Hz), 4.50-4.65 (3H, m), 5.06 (2H, q, J=8.9 Hz), 6.05 (1H, d, J=1.9 Hz), 6.09-6.17 (2H, m), 6.52 (1H, d, J=7.5 Hz), 6.86 (1H, d, J=7.9 Hz), 6.92 (1H, d, J=7.9 Hz), 6.97-7.06 (1H, m), 7.21-7.27 (1H, m), 7.35 (1H, t, J=7.5 Hz), 7.43-7.49 (1H, m).

MS m/z 556 (M+H)⁺.

### Example 181

### {(3S)-6-[(3-{2-ethoxy-4-[3-(methylsulfonyl)propoxy]-1H-benzimidazol-1-yl}-2-methylbenzyl)amino]-2,3-dihydro-1-benzofuran-3-yl}acetic acid

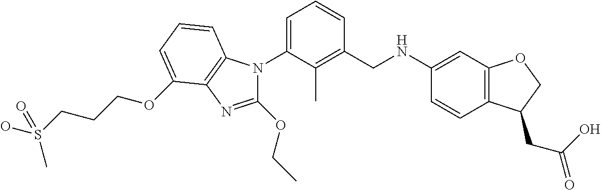

Methyl [(3S)-6-{[3-(2-ethoxy-4-hydroxy-1H-benzimidazol-1-yl)-2-methylbenzyl](trifluoroacetyl)amino}-2,3-dihydro-1-benzofuran-3-yl]acetate (116 mg, 0.2 mmol), 3-(methylsulfonyl)propyl p-toluenesulfonate (88 mg, 0.3 mmol), potassium carbonate (55 mg, 0.4 mmol) were suspended in N,N-dimethylformamide (3 mL) and the suspension was stirred at 60° C. for 2 hr. The reaction mixture was allowed to cool, diluted with saturated brine, and the mixture was extracted with ethyl acetate. The extract was dried over anhydrous magnesium sulfate, and the volatile component was evaporated under reduced pressure. The residue was purified by silica gel column chromatography (hexane:ethyl acetate=90:10-40:60) to give an oil (130 mg). The obtained oil was dissolved in methanol (5 mL)-tetrahydrofuran (5 mL), 1 M aqueous sodium hydroxide solution (0.5 mL) was added, and the mixture was stirred at 50° C. for 1 hr. Water (10 mL) was added, and the volatile component was evaporated under reduced pressure. 1 M Aqueous hydrochloric acid solution (0.5 mL) was added, and the mixture was diluted with saturated brine, extracted with ethyl acetate. The extract was washed with saturated brine and dried over anhydrous magnesium sulfate, and the volatile component was evaporated under reduced pressure to give the title compound (77 mg, yield 65%) as a white solid.

¹H NMR (300 MHz, DMSO-d₆) δ 1.32 (3H, t, J=7.0 Hz), 1.93 (3H, s), 2.17-2.29 (2H, m), 2.36-2.47 (1H, m), 2.57-2.69 (1H, m), 3.05 (3H, s), 3.34-3.39 (3H, m), 3.54-3.67 (1H, m), 4.05-4.14 (1H, m), 4.22-4.40 (4H, m), 4.46-4.65 (3H, m), 6.00-6.19 (3H, m), 6.43 (1H, d, J=7.9 Hz), 6.76 (1H, d, J=7.9 Hz), 6.88-7.04 (2H, m), 7.20-7.26 (1H, m), 7.34 (1H, t, J=7.5 Hz), 7.42-7.48 (1H, m).

MS m/z 594 (M+H)⁺.

### Example 182

### [(3S)-6-({3-[4-(2-hydroxypropan-2-yl)piperidin-1-yl]-2-methylbenzyl}amino)-2,3-dihydro-1-benzofuran-3-yl]acetic acid

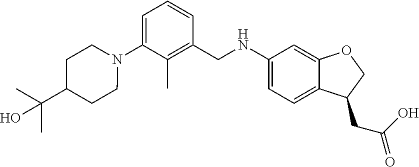

Methyl {(3S)-6-[(3-bromo-2-methylbenzyl)(trifluoroacetyl)amino]-2,3-dihydro-1-benzofuran-3-yl}acetate (486 mg, 1.0 mmol), 2-(piperidin-4-yl)propan-2-ol (286 mg, 2.0 mmol), tris(dibenzylideneacetone)dipalladium (46 mg, 0.05 mmol), 2-(dicyclohexylphosphino)-2′,4′,6′-triisopropyl-1′,1′-biphenyl (48 mg, 0.1 mmol) and tripotassium phosphate (424 mg, 2.0 mmol) were suspended in toluene (40 mL), and the suspension was stirred under an argon atmosphere overnight at 100° C. The reaction mixture was allowed to cool, diluted with water and extracted with ethyl acetate. The extract was washed with saturated brine, dried over anhydrous magnesium sulfate, and the volatile component was evaporated under reduced pressure. The residue was purified by silica gel column chromatography (hexane:ethyl acetate=90:10-40:60) to give an oil (72 mg). The obtained oil was dissolved in methanol (2 mL)-tetrahydrofuran (2 mL), 1 M aqueous sodium hydroxide solution (0.5 mL) was added, and the mixture was stirred at 50° C. for 1 hr. The volatile component was evaporated under reduced pressure, and 1 M aqueous hydrochloric acid solution (0.5 mL) was added. The mixture was diluted with saturated brine, extracted with ethyl acetate. The extract was washed with saturated brine and dried over anhydrous magnesium sulfate and the volatile component was evaporated under reduced pressure. The residue was crystallized from ethyl acetate-heptane to give the title compound (40 mg, yield 5%) as a white solid.

¹H NMR (300 MHz, DMSO-d₆) δ 1.09 (6H, s), 1.19-1.50 (3H, m), 1.77 (2H, d, J=11.3 Hz), 2.22 (3H, s), 2.34-2.46 (1H, m), 2.46-2.56 (1H, m), 2.57-2.66 (1H, m), 3.05 (2H, d, J=11.3 Hz), 3.50-3.65 (1H, m), 4.00-4.18 (4H, m), 4.57 (1H, t, J=8.9 Hz), 5.87-6.00 (2H, m), 6.08 (1H, dd, J=7.9, 1.9 Hz), 6.84-6.99 (3H, m), 7.02-7.10 (1H, m), 12.28 (1H, br s).

### Example 183

{(3S)-6-[(3-{2-ethyl-4-[3-(methylsulfonyl)propoxy]-1H-benzimidazol-1-yl}-2-methylbenzyl)amino]-2,3-dihydro-1-benzofuran-3-yl}acetic acid

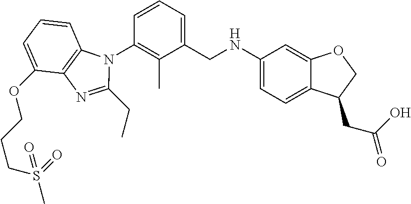

In the same manner as in Example 181, the title compound was synthesized using methyl [(3S)-6-{[3-(2-ethyl-4-hydroxy-1H-benzimidazol-1-yl)-2-methylbenzyl](trifluoroacetyl)amino}-2,3-dihydro-1-benzofuran-3-yl]acetate.

MS m/z 578 (M+H)⁺.

### Example 184

### methyl [(3S)-6-{[3-(6-methoxypyridin-3-yl)-2,4-dimethylbenzyl]amino}-2,3-dihydro-1-benzofuran-3-yl]acetate

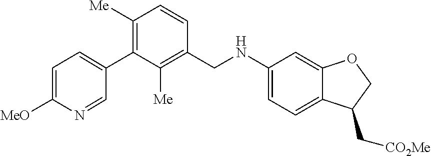

Methyl {(3S)-6-[(3-bromo-2,4-dimethylbenzyl)amino]-2,3-dihydro-1-benzofuran-3-yl}acetate (0.204 g, 0.505 mmol) and (6-methoxypyridin-3-yl)boronic acid (93.0 mg, 0.605 mmol) were suspended in a mixed solution of 2 M aqueous sodium carbonate solution (0.606 mL) and toluene (3 mL) and, after argon substitution, tris(dibenzylideneacetone)dipalladium (0) (18.5 mg, 0.0200 mmol) and 2-dicyclohexylphosphino-2′,6′-dimethoxybiphenyl (34.2 mg, 0.0810 mmol) were added. The reaction mixture was stirred under an argon atmosphere at 100° C. for 5 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-65:35) to give the title compound (0.184 g, yield 84%) as a colorless oil.

¹H NMR (300 MHz, CDCl₃) δ 2.03 (6H, s), 2.54 (1H, dd, J=16.4, 9.2 Hz), 2.74 (1H, dd, J=16.4, 5.5 Hz), 3.66-3.84 (4H, m), 3.88 (1H, br s), 4.00 (3H, s), 4.17-4.29 (3H, m), 4.71 (1H, t, J=8.9 Hz), 6.11-6.21 (2H, m), 6.84 (1H, dd, J=8.5, 0.9 Hz), 6.92-6.98 (1H, m), 7.10 (1H, d, J=7.7 Hz), 7.23-7.29 (1H, m), 7.37 (1H, dd, J=8.5, 2.4 Hz), 7.95 (1H, dd, J=2.4, 0.9 Hz).

MS m/z 433 (M+H)⁺.

### Example 185

### [(3S)-6-{[3-(6-methoxypyridin-3-yl)-2,4-dimethylbenzyl]amino}-2,3-dihydro-1-benzofuran-3-yl]acetic acid

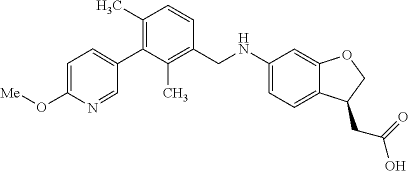

To a mixed solution of methyl [(3S)-6-{[3-(6-methoxypyridin-3-yl)-2,4-dimethylbenzyl]amino}-2,3-dihydro-1-benzofuran-3-yl]acetate (0.184 g, 0.425 mmol) in tetrahydrofuran (2 mL) and methanol (1 mL) was added 1 M aqueous sodium hydroxide solution (1.28 mL), and the mixture was stirred at room temperature for 1.5 hr. The reaction mixture was concentrated under reduced pressure, water was added to dissolve the residue, and 1 M hydrochloric acid (1.28 mL) was slowly added. The obtained precipitate was collected by filtration, washed with water, and dried to give the title compound (0.153 g, yield 86%) as a white solid.

¹H NMR (300 MHz, CDCl₃) δ 2.03 (6H, s), 2.59 (1H, dd, J=16.7, 9.5 Hz), 2.79 (1H, dd, J=16.7, 5.3 Hz), 3.70-3.84 (1H, m), 4.00 (3H, s), 4.20-4.30 (3H, m), 4.72 (1H, t, J=8.9 Hz), 6.11-6.22 (2H, m), 6.84 (1H, d, J=8.3 Hz), 6.98 (1H, d, J=8.0 Hz), 7.10 (1H, d, J=8.0 Hz), 7.22-7.29 (1H, m), 7.37 (1H, dd, J=8.3, 2.3 Hz), 7.95 (1H, d, J=1.9 Hz).

MS m/z 419 (M+H)⁺.

### Example 186

### methyl [(3S)-6-({3-[1-(difluoromethyl)-3,5-dimethyl-1H-pyrazol-4-yl]-2,4-dimethylbenzyl}amino)-2,3-dihydro-1-benzofuran-3-yl]acetate

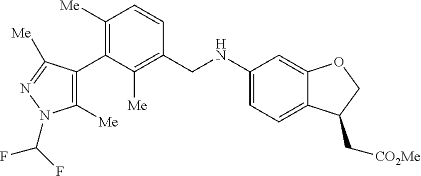

In the same manner as in Example 184, the title compound (0.195 g, yield 87%) was obtained as a colorless oil from methyl {(3S)-6-[(3-bromo-2,4-dimethylbenzyl)amino]-2,3-dihydro-1-benzofuran-3-yl}acetate and 1-(difluoromethyl)-3,5-dimethyl-4-(4,4,5,5-tetramethyl-1,3,2-dioxaborolan-2-yl)-1H-pyrazole.

¹H NMR (300 MHz, CDCl₃) δ 1.94-2.02 (9H, m), 2.16 (3H, s), 2.54 (1H, dd, J=16.3, 9.1 Hz), 2.74 (1H, dd, J=16.3, 5.3 Hz), 3.67-3.93 (5H, m), 4.18-4.28 (3H, m), 4.72 (1H, t, J=8.9 Hz), 6.11-6.22 (2H, m), 6.91-7.41 (4H, m).

MS m/z 470 (M+H)⁺.

### Example 187

### [(3S)-6-({3-[1-(difluoromethyl)-3,5-dimethyl-1H-pyrazol-4-yl]-2,4-dimethylbenzyl}amino)-2,3-dihydro-1-benzofuran-3-yl]acetic acid

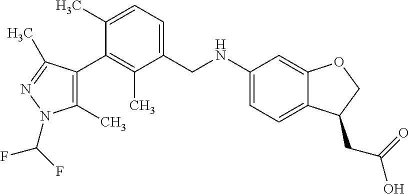

In the same manner as in Example 185, the title compound was obtained as a white solid from methyl [(3S)-6-({3-[1-(difluoromethyl)-3,5-dimethyl-1H-pyrazol-4-yl]-2,4-dimethylbenzyl}amino)-2,3-dihydro-1-benzofuran-3-yl]acetate. yield 82%.

¹H NMR (300 MHz, CDCl₃) δ 1.99 (6H, s), 2.01 (3H, s), 2.16 (3H, s), 2.60 (1H, dd, J=16.7, 9.5 Hz), 2.80 (1H, dd, J=16.7, 5.3 Hz), 3.71-3.86 (1H, m), 4.20-4.31 (3H, m), 4.73 (1H, t, J=9.1 Hz), 6.11-6.23 (2H, m), 6.98 (1H, d, J=8.0 Hz), 7.01-7.44 (3H, m).

MS m/z 456 (M+H)⁺.

### Example 188

### methyl [(3S)-6-{[3-(6-methoxypyridin-3-yl)-2-methylbenzyl]amino}-2,3-dihydro-1-benzofuran-3-yl]acetate

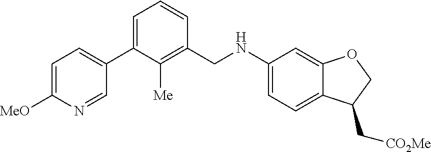

In the same manner as in Example 184, the title compound was obtained as a pale-red oil from methyl {(3S)-6-[(3-bromo-2-methylbenzyl)amino]-2,3-dihydro-1-benzofuran-3-yl}acetate and (6-methoxypyridin-3-yl)boronic acid. yield 100%.

¹H NMR (300 MHz, CDCl₃) δ 2.23 (3H, s), 2.54 (1H, dd, J=16.3, 9.5 Hz), 2.74 (1H, dd, J=16.3, 5.7 Hz), 3.69-3.84 (4H, m), 3.93 (1H, br s), 3.99 (3H, s), 4.23 (1H, dd, J=9.3, 5.9 Hz), 4.29 (2H, s), 4.72 (1H, t, J=8.9 Hz), 6.12-6.21 (2H, m), 6.78-6.84 (1H, m), 6.95 (1H, d, J=8.0 Hz), 7.12-7.28 (2H, m), 7.33-7.40 (1H, m), 7.53 (1H, dd, J=8.5, 2.5 Hz), 8.09-8.13 (1H, m).

MS m/z 419 (M+H)⁺.

### Example 189

### [(3S)-6-{[3-(6-methoxypyridin-3-yl)-2-methylbenzyl]amino}-2,3-dihydro-1-benzofuran-3-yl]acetic acid

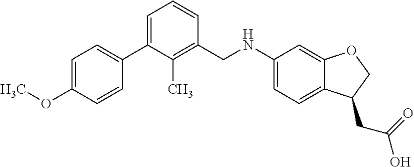

In the same manner as in Example 185, the title compound was obtained as a white solid from methyl [(3S)-6-{[3-(6-methoxypyridin-3-yl)-2-methylbenzyl]amino}-2,3-dihydro-1-benzofuran-3-yl]acetate. yield 59%.

¹H NMR (300 MHz, DMSO-d₆) δ 2.18 (3H, s), 2.42 (1H, dd, J=16.3, 9.1 Hz), 2.62 (1H, dd, J=16.3, 5.7 Hz), 3.51-3.68 (1H, m), 3.90 (3H, s), 4.08 (1H, dd, J=9.1, 6.8 Hz), 4.21 (2H, d, J=4.5 Hz), 4.59 (1H, t, J=8.9 Hz), 5.96-6.17 (3H, m), 6.90 (2H, d, J=8.7 Hz), 7.05-7.35 (3H, m), 7.68 (1H, dd, J=8.5, 2.5 Hz), 8.11 (1H, d, J=2.3 Hz), 12.29 (1H, br s).

MS m/z 405 (M+H)⁺.

### Example 190

### methyl [(3S)-6-{[3-(1,4-dioxaspiro[4.5]dec-7-en-8-yl)-2,4-dimethylbenzyl]amino}-2,3-dihydro-1-benzofuran-3-yl]acetate

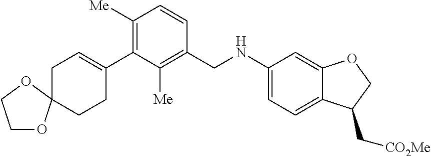

3-(1,4-Dioxaspiro[4.5]dec-7-en-8-yl)-2,4-dimethylbenzaldehyde (0.254 g, 0.933 mmol) and methyl [(3S)-6-amino-2,3-dihydro-1-benzofuran-3-yl]acetate (0.193 g, 0.933 mmol) were dissolved in acetic acid (0.160 mL, 2.80 mmol) and acetonitrile (5 mL), sodium triacetoxyborohydride (0.396 g, 1.87 mmol) was added at 0° C., and the mixture was stirred at room temperature for 4 hr. The reaction mixture was neutralized with saturated aqueous sodium hydrogen carbonate, and the mixture was extracted with ethyl acetate. The extract was dried over sodium sulfate, and concentrated under reduced pressure. The residue was purified by silica gel column chromatography (hexane:ethyl acetate=100:0-75:25) to give the title compound (0.225 g, yield 52%) as a colorless oil.

¹H NMR (300 MHz, CDCl₃) δ 1.90 (2H, t, J=6.6 Hz), 2.23 (6H, s), 2.25-2.36 (2H, m), 2.40-2.46 (2H, m), 2.53 (1H, dd, J=16.3, 9.1 Hz), 2.73 (1H, dd, J=16.3, 5.3 Hz), 3.68-3.87 (5H, m), 4.00-4.06 (4H, m), 4.22 (3H, dd, J=9.1, 6.1 Hz), 4.71 (1H, t, J=8.9 Hz), 5.31-5.39 (1H, m), 6.10-6.20 (2H, m), 6.93 (1H, d, J=8.0 Hz), 6.96-7.03 (1H, m), 7.12 (1H, d, J=7.6 Hz).

MS m/z 464 (M+H)⁺.

### Example 191

### [(3S)-6-{[3-(1,4-dioxaspiro[4.5]dec-7-en-8-yl)-2,4-dimethylbenzyl]amino}-2,3-dihydro-1-benzofuran-3-yl]acetic acid

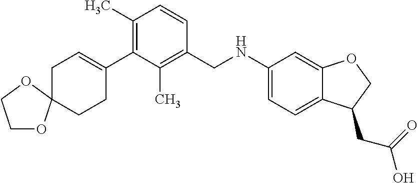

In the same manner as in Example 185, the title compound was obtained as a white solid from methyl [(3S)-6-{[3-(1,4-dioxaspiro[4.5]dec-7-en-8-yl)-2,4-dimethylbenzyl]amino}-2,3-dihydro-1-benzofuran-3-yl]acetate. yield 90%.

¹H NMR (300 MHz, CDCl₃) δ 1.91 (2H, t, J=6.5 Hz), 2.23 (6H, s), 2.26-2.36 (2H, m), 2.40-2.48 (2H, m), 2.59 (1H, dd, J=16.8, 9.2 Hz), 2.79 (1H, dd, J=16.8, 5.3 Hz), 3.71-3.85 (1H, m), 3.99-4.07 (4H, m), 4.19 (2H, s), 4.25 (1H, dd, J=9.1, 5.9 Hz), 4.72 (1H, t, J=8.9 Hz), 5.32-5.40 (1H, m), 6.11-6.20 (2H, m), 6.93-7.04 (2H, m), 7.12 (1H, d, J=7.7 Hz).

MS m/z 450 (M+H)⁺.

### Example 192

### methyl [(3S)-6-{[3-(1,4-dioxa-8-azaspiro[4.5]dec-8-yl)-2,4-dimethylbenzyl]amino}-2,3-dihydro-1-benzofuran-3-yl]acetate

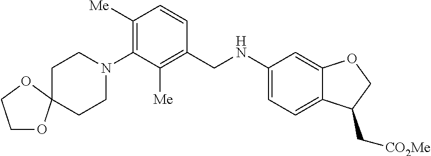

In the same manner as in Example 190, the title compound was obtained as a yellow oil from 3-(1,4-dioxa-8-azaspiro[4.5]dec-8-yl)-2,4-dimethylbenzaldehyde and methyl [(3S)-6-amino-2,3-dihydro-1-benzofuran-3-yl]acetate. yield 35%.

¹H NMR (300 MHz, CDCl₃) δ 1.74-1.93 (4H, m), 2.30 (3H, s), 2.32 (3H, s), 2.53 (1H, dd, J=16.3, 9.5 Hz), 2.73 (1H, dd, J=16.3, 5.7 Hz), 3.05-3.29 (4H, m), 3.67-3.87 (5H, m), 4.01 (4H, s), 4.14-4.26 (3H, m), 4.71 (1H, t, J=8.9 Hz), 6.09-6.18 (2H, m), 6.90-7.05 (3H, m).

MS m/z 467 (M+H)⁺.

### Example 193

### [(3S)-6-{[3-(1,4-dioxa-8-azaspiro[4.5]dec-8-yl)-2,4-dimethylbenzyl]amino}-2,3-dihydro-1-benzofuran-3-yl]acetic acid

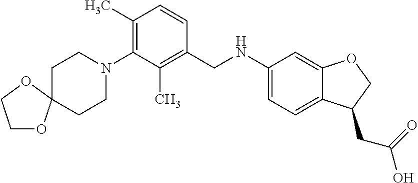

In the same manner as in Example 185, the title compound was obtained as a yellow solid from methyl [(3S)-6-{[3-(1,4-dioxa-8-azaspiro[4.5]dec-8-yl)-2,4-dimethylbenzyl]amino}-2,3-dihydro-1-benzofuran-3-yl]acetate. yield 87%.

¹H NMR (300 MHz, CDCl₃) δ 1.74-1.93 (4H, m), 2.30 (3H, s), 2.32 (3H, s), 2.59 (1H, dd, J=16.6, 9.2 Hz), 2.79 (1H, dd, J=16.8, 5.3 Hz), 3.04-3.31 (4H, m), 3.70-3.84 (1H, m), 4.01 (4H, s), 4.16 (2H, s), 4.24 (1H, dd, J=9.2, 6.0 Hz), 4.71 (1H, t, J=9.0 Hz), 6.07-6.20 (2H, m), 6.90-7.05 (3H, m).

MS m/z 453 (M+H)⁺.

### Example 194

### methyl [(3S)-6-{[2,4-dimethyl-3-(6-morpholin-4-ylpyridin-3-yl)benzyl]amino}-2,3-dihydro-1-benzofuran-3-yl]acetate

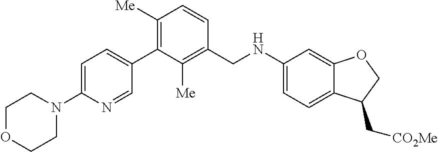

In the same manner as in Example 184, the title compound was obtained as a yellow non-crystalline powder from methyl {(3S)-6-[(3-bromo-2,4-dimethylbenzyl)amino]-2,3-dihydro-1-benzofuran-3-yl}acetate and (6-morpholin-4-ylpyridin-3-yl)boronic acid. yield 91%.

¹H NMR (300 MHz, CDCl₃) δ 2.02-2.08 (6H, m), 2.53 (1H, dd, J=16.3, 9.5 Hz), 2.73 (1H, dd, J=16.3, 5.3 Hz), 3.51-3.60 (4H, m), 3.68-3.83 (4H, m), 3.83-3.92 (5H, m), 4.18-4.27 (3H, m), 4.71 (1H, t, J=8.9 Hz), 6.11-6.21 (2H, m), 6.73 (1H, d, J=8.0 Hz), 6.94 (1H, d, J=8.0 Hz), 7.09 (1H, d, J=8.0 Hz), 7.21-7.27 (1H, m), 7.31 (1H, dd, J=8.7, 2.3 Hz), 8.00 (1H, d, J=1.5 Hz).

MS m/z 488 (M+H)⁺.

### Example 195

### [(3S)-6-({2,4-dimethyl-3-[6-(morpholin-4-yl)pyridin-3-yl]benzyl}amino)-2,3-dihydro-1-benzofuran-3-yl]acetic acid

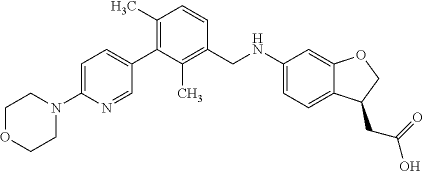

In the same manner as in Example 185, the title compound was obtained as a beige solid from methyl [(3S)-6-{[2,4-dimethyl-3-(6-morpholin-4-ylpyridin-3-yl)benzyl]amino}-2,3-dihydro-1-benzofuran-3-yl]acetate. yield 86%.

¹H NMR (300 MHz, CDCl₃) δ 2.05 (6H, s), 2.59 (1H, dd, J=16.6, 9.2 Hz), 2.79 (1H, dd, J=16.8, 5.3 Hz), 3.50-3.60 (4H, m), 3.70-3.92 (5H, m), 4.18-4.30 (3H, m), 4.72 (1H, t, J=8.9 Hz), 6.12-6.22 (2H, m), 6.75 (1H, d, J=0.6 Hz), 6.98 (1H, d, J=8.1 Hz), 7.09 (1H, d, J=7.9 Hz), 7.20-7.27 (1H, m), 7.32 (1H, dd, J=8.6, 2.4 Hz), 8.01 (1H, d, J=2.1 Hz).

MS m/z 474 (M+H)⁺.

### Example 196

### methyl [(3S)-6-{[3-(2,4-dimethoxypyrimidin-5-yl)-2,4-dimethylbenzyl]amino}-2,3-dihydro-1-benzofuran-3-yl]acetate

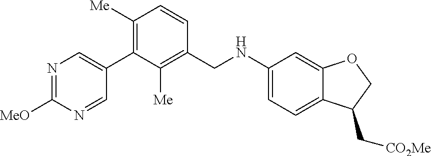

In the same manner as in Example 184, the title compound was obtained as a colorless oil from methyl {(3S)-6-[(3-bromo-2,4-dimethylbenzyl)amino]-2,3-dihydro-1-benzofuran-3-yl}acetate and (2,4-dimethoxypyrimidin-5-yl)boronic acid. yield 65%.

¹H NMR (300 MHz, CDCl₃) δ 2.02 (6H, s), 2.54 (1H, dd, J=16.3, 9.5 Hz), 2.74 (1H, dd, J=16.3, 5.3 Hz), 3.67-3.83 (4H, m), 3.87 (1H, br s), 3.95 (3H, s), 4.06 (3H, s), 4.17-4.29 (3H, m), 4.71 (1H, t, J=8.9 Hz), 6.11-6.21 (2H, m), 6.94 (1H, d, J=8.0 Hz), 7.11 (1H, d, J=8.0 Hz), 7.23-7.31 (1H, m), 7.98 (1H, s).

MS m/z 464 (M+H)⁺.

### Example 197

### [(3S)-6-{[3-(2,4-dimethoxypyrimidin-5-yl)-2,4-dimethylbenzyl]amino}-2,3-dihydro-1-benzofuran-3-yl]acetic acid

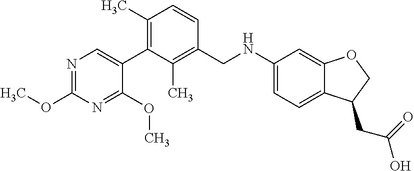

In the same manner as in Example 185, the title compound was obtained as a white solid from methyl [(3S)-6-{[3-(2,4-dimethoxypyrimidin-5-yl)-2,4-dimethylbenzyl]amino}-2,3-dihydro-1-benzofuran-3-yl]acetate. yield 83%.

¹H NMR (300 MHz, CDCl₃) δ 2.02 (6H, s), 2.59 (1H, dd, J=16.6, 9.2 Hz), 2.79 (1H, dd, J=16.8, 5.3 Hz), 3.70-3.85 (1H, m), 3.95 (3H, s), 4.06 (3H, s), 4.18-4.30 (3H, m), 4.72 (1H, t, J=8.9 Hz), 6.11-6.22 (2H, m), 6.98 (1H, d, J=7.9 Hz), 7.10 (1H, d, J=7.9 Hz), 7.23-7.31 (1H, m), 8.00 (1H, s).

MS m/z 450 (M+H)⁺.

### Example 198

### methyl [(3S)-6-{[3-(3,4-dihydro-2H-pyran-6-yl)-2,4-dimethylbenzyl]amino}-2,3-dihydro-1-benzofuran-3-yl]acetate

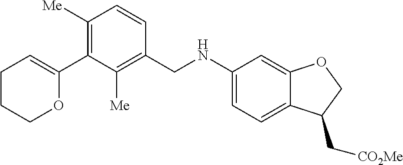

In the same manner as in Example 184, the title compound was obtained as a pale-yellow oil from methyl {(3S)-6-[(3-bromo-2,4-dimethylbenzyl)amino]-2,3-dihydro-1-benzofuran-3-yl}acetate and 3,4-dihydro-2H-pyran-6-ylboronic acid. yield 38%.

¹H NMR (300 MHz, CDCl₃) δ 1.89-2.02 (2H, m), 2.15-2.26 (2H, m), 2.31 (6H, s), 2.53 (1H, dd, J=16.3, 9.5 Hz), 2.73 (1H, dd, J=16.3, 5.3 Hz), 3.65-3.85 (5H, m), 4.06-4.28 (5H, m), 4.62-4.77 (2H, m), 6.08-6.17 (2H, m), 6.92 (1H, d, J=8.7 Hz), 7.00 (1H, d, J=8.0 Hz), 7.16 (1H, d, J=7.6 Hz).

MS m/z 408 (M+H)⁺.

### Example 199

### [(3S)-6-{[3-(3,4-dihydro-2H-pyran-6-yl)-2,4-dimethylbenzyl]amino}-2,3-dihydro-1-benzofuran-3-yl]acetic acid

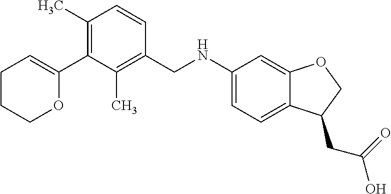

In the same manner as in Example 185, the title compound was obtained as a white solid from methyl [(3S)-6-{[3-(3,4-dihydro-2H-pyran-6-yl)-2,4-dimethylbenzyl]amino}-2,3-dihydro-1-benzofuran-3-yl]acetate. yield 77%.

¹H NMR (300 MHz, CDCl₃) δ 1.89-2.03 (2H, m), 2.15-2.26 (2H, m), 2.31 (6H, s), 2.58 (1H, dd, J=16.6, 9.2 Hz), 2.79 (1H, dd, J=16.6, 5.1 Hz), 3.62-3.85 (2H, m), 4.09-4.29 (4H, m), 4.62-4.78 (2H, m), 6.09-6.18 (2H, m), 6.91-7.04 (2H, m), 7.16 (1H, d, J=7.9 Hz).

MS m/z 394 (M+H)⁺.

### Example 200

### methyl [(3S)-6-{[2-methyl-3-(2-methylimidazo[1,2-a]pyridin-3-yl)benzyl]amino}-2,3-dihydro-1-benzofuran-3-yl]acetate

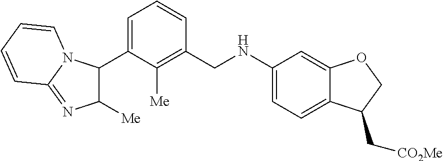

Methyl [(3S)-6-{[2-methyl-3-(4,4,5,5-tetramethyl-1,3,2-dioxaborolan-2-yl)benzyl]amino}-2,3-dihydro-1-benzofuran-3-yl]acetate (0.560 g, 1.28 mmol) and 3-bromo-2-methylimidazo[1,2-a]pyridine (0.351 g, 1.67 mmol) were suspended in a mixed solution of 2 M aqueous sodium carbonate solution (1.54 mL) and toluene (7 mL). After argon substitution, tris(dibenzylideneacetone)dipalladium (0) (46.9 mg, 0.0510 mmol) and 2-dicyclohexylphosphino-2′,6′-dimethoxybiphenyl (87.0 mg, 0.205 mmol) were added. The reaction mixture was stirred under an argon atmosphere at 105° C. for 20 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=90:10-0:100) to give the title compound (0.186 g, yield 33%) as a yellow oil.

¹H NMR (300 MHz, CDCl₃) δ 2.06 (3H, s), 2.36 (3H, s), 2.55 (1H, dd, J=16.4, 9.2 Hz), 2.75 (1H, dd, J=16.2, 5.3 Hz), 3.68-3.86 (4H, m), 4.00 (1H, br s), 4.24 (1H, dd, J=9.2, 6.0 Hz), 4.34 (2H, s), 4.73 (1H, t, J=8.9 Hz), 6.15 (1H, d, J=2.1 Hz), 6.20 (1H, dd, J=7.9, 2.1 Hz), 6.67-6.75 (1H, m), 6.97 (1H, d, J=8.1 Hz), 7.12-7.26 (2H, m), 7.31 (1H, t, J=7.6 Hz), 7.48 (1H, dd, J=7.4, 1.2 Hz), 7.53-7.62 (2H, m).

MS m/z 442 (M+H)⁺.

### Example 201

### [(3S)-6-{[2-methyl-3-(2-methylimidazo[1,2-a]pyridin-3-yl)benzyl]amino}-2,3-dihydro-1-benzofuran-3-yl]acetic acid

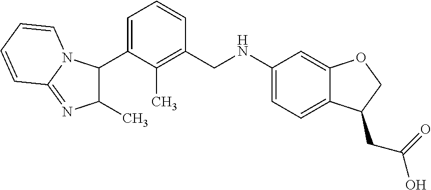

In the same manner as in Example 185, the title compound was obtained as a beige solid from methyl [(3S)-6-{[2-methyl-3-(2-methylimidazo[1,2-a]pyridin-3-yl)benzyl]amino}-2,3-dihydro-1-benzofuran-3-yl]acetate. yield 52%.

¹H NMR (300 MHz, CDCl₃) δ 2.06 (3H, s), 2.37 (3H, s), 2.61 (1H, dd, J=16.3, 9.5 Hz), 2.83 (1H, dd, J=16.3, 4.9 Hz), 3.78-3.94 (1H, m), 4.26-4.39 (3H, m), 4.79 (1H, t, J=8.9 Hz), 6.13-6.25 (2H, m), 6.75-6.83 (1H, m), 7.04 (1H, d, J=8.0 Hz), 7.18-7.37 (3H, m), 7.51 (1H, d, J=6.4 Hz), 7.59 (1H, d, J=6.8 Hz), 7.78 (1H, d, J=9.1 Hz).

MS m/z 428 (M+H)⁺.

### Example 202

### methyl [(3S)-6-({2-methyl-3-[2-(trifluoromethyl)-1H-benzimidazol-1-yl]benzyl}amino)-2,3-dihydro-1-benzofuran-3-yl]acetate

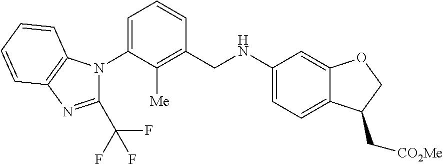

In the same manner as in Example 190, the title compound was obtained as a colorless oil from 2-methyl-3-[2-(trifluoromethyl)-1H-benzimidazol-1-yl]benzaldehyde and methyl [(3S)-6-amino-2,3-dihydro-1-benzofuran-3-yl]acetate. yield 80%.

¹H NMR (300 MHz, CDCl₃) δ 1.90 (3H, s), 2.55 (1H, dd, J=16.3, 9.1 Hz), 2.75 (1H, dd, J=16.3, 5.3 Hz), 3.68-3.86 (4H, m), 4.02 (1H, br s), 4.24 (1H, dd, J=9.1, 6.1 Hz), 4.35 (2H, s), 4.73 (1H, t, J=8.9 Hz), 6.10-6.23 (2H, m), 6.93-7.05 (2H, m), 7.22-7.29 (1H, m), 7.31-7.47 (3H, m), 7.59 (1H, d, J=7.6 Hz), 7.93-8.00 (1H, m).

MS m/z 496 (M+H)⁺.

### Example 203

### [(3S)-6-({2-methyl-3-[2-(trifluoromethyl)-1H-benzimidazol-1-yl]benzyl}amino)-2,3-dihydro-1-benzofuran-3-yl]acetic acid

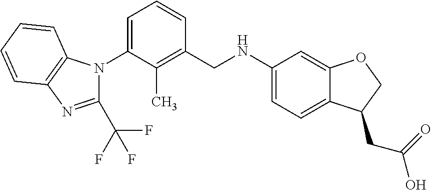

In the same manner as in Example 185, the title compound was obtained as a white solid from methyl [(3S)-6-({2-methyl-3-[2-(trifluoromethyl)-1H-benzimidazol-1-yl]benzyl}amino)-2,3-dihydro-1-benzofuran-3-yl]acetate. yield 83%.

¹H NMR (300 MHz, CDCl₃) δ 1.90 (3H, s), 2.61 (1H, dd, J=16.7, 9.1 Hz), 2.81 (1H, dd, J=16.7, 5.3 Hz), 3.71-3.88 (1H, m), 4.27 (1H, dd, J=9.5, 6.1 Hz), 4.35 (2H, s), 4.74 (1H, t, J=9.1 Hz), 6.12-6.22 (2H, m), 6.96-7.04 (2H, m), 7.22-7.29 (1H, m), 7.32-7.46 (3H, m), 7.56-7.62 (1H, m), 7.97 (1H, d, J=6.8 Hz).

MS m/z 482 (M+H)⁺.

### Example 204

### methyl [(3S)-6-{[3-(2-cyclopropyl-1H-benzimidazol-1-yl)-2-methylbenzyl]amino}-2,3-dihydro-1-benzofuran-3-yl]acetate

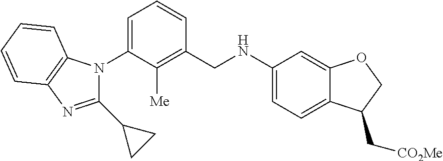

In the same manner as in Example 190, the title compound was obtained as an orange non-crystalline powder from 3-(2-cyclopropyl-1H-benzimidazol-1-yl)-2-methylbenzaldehyde and methyl [(3S)-6-amino-2,3-dihydro-1-benzofuran-3-yl]acetate. yield 75%.

¹H NMR (300 MHz, CDCl₃) δ 0.94-1.05 (2H, m), 1.26-1.34 (2H, m), 1.57-1.68 (1H, m), 1.99 (3H, s), 2.55 (1H, dd, J=16.4, 9.2 Hz), 2.75 (1H, dd, J=16.4, 5.5 Hz), 3.69-3.86 (4H, m), 4.05 (1H, br s), 4.24 (1H, dd, J=9.2, 6.0 Hz), 4.36 (2H, s), 4.73 (1H, t, J=8.9 Hz), 6.14 (1H, d, J=2.1 Hz), 6.20 (1H, dd, J=8.1, 2.1 Hz), 6.90 (1H, d, J=7.9 Hz), 6.97 (1H, d, J=8.1 Hz), 7.11-7.19 (1H, m), 7.20-7.30 (2H, m), 7.36 (1H, t, J=7.7 Hz), 7.51-7.58 (1H, m), 7.69-7.76 (1H, m).

MS m/z 468 (M+H)⁺.

### Example 205

### [(3S)-6-{[3-(2-cyclopropyl-1H-benzimidazol-1-yl)-2-methylbenzyl]amino}-2,3-dihydro-1-benzofuran-3-yl]acetic acid

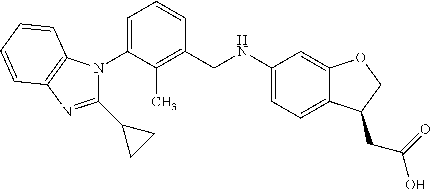

In the same manner as in Example 185, the title compound was obtained as a white solid from methyl [(3S)-6-{[3-(2-cyclopropyl-1H-benzimidazol-1-yl)-2-methylbenzyl]amino}-2,3-dihydro-1-benzofuran-3-yl]acetate. yield 88%.

¹H NMR (300 MHz, CDCl₃) δ 0.95-1.07 (2H, m), 1.28-1.39 (2H, m), 1.57-1.70 (1H, m), 1.99 (3H, s), 2.61 (1H, dd, J=16.7, 9.5 Hz), 2.82 (1H, dd, J=16.5, 5.5 Hz), 3.76-3.90 (1H, m), 4.29 (1H, dd, J=9.3, 6.2 Hz), 4.37 (2H, s), 4.77 (1H, t, J=8.9 Hz), 6.11-6.25 (2H, m), 6.90 (1H, d, J=8.0 Hz), 7.03 (1H, d, J=8.3 Hz), 7.12-7.21 (1H, m), 7.21-7.30 (2H, m), 7.37 (1H, t, J=7.8 Hz), 7.56 (1H, d, J=7.2 Hz), 7.77 (1H, d, J=7.6 Hz).

MS m/z 454 (M+H)⁺.

### Example 206

### [(3S)-6-({2-methyl-3-[6-(morpholin-4-yl)pyridin-3-yl]-4-(trifluoromethyl)benzyl}amino)-2,3-dihydro-1-benzofuran-3-yl]acetic acid

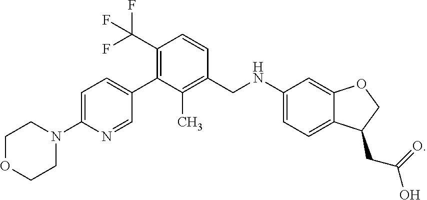

To a mixed solution of methyl [(3S)-6-{[2-methyl-3-(6-morpholin-4-ylpyridin-3-yl)-4-(trifluoromethyl)benzyl](trifluoroacetyl)amino}-2,3-dihydro-1-benzofuran-3-yl]acetate (0.248 g, 0.389 mmol) in tetrahydrofuran (2 mL) and methanol (1 mL) was added 1 M aqueous sodium hydroxide solution (1.56 mL), and the mixture was stirred at room temperature for 1.5 hr. The reaction mixture was concentrated under reduced pressure, water was added to dissolve the residue, and 1 M hydrochloric acid (1.17 mL) was slowly added. The obtained precipitate was collected by filtration, washed with water, and dried to give the title compound (0.129 g, yield 63%) as a beige solid.

¹H NMR (300 MHz, CDCl₃) δ 2.04 (3H, s), 2.59 (1H, dd, J=16.8, 9.2 Hz), 2.79 (1H, dd, J=16.8, 5.3 Hz), 3.54-3.64 (4H, m), 3.71-3.84 (1H, m), 3.84-3.92 (4H, m), 4.25 (1H, dd, J=9.1, 6.1 Hz), 4.32 (2H, s), 4.73 (1H, t, J=9.0 Hz), 6.09 (1H, d, J=2.1 Hz), 6.16 (1H, dd, J=8.1, 2.1 Hz), 6.73 (1H, d, J=8.9 Hz), 6.98 (1H, d, J=8.1 Hz), 7.36 (1H, dd, J=8.7, 1.9 Hz), 7.47 (1H, d, J=8.1 Hz), 7.56 (1H, d, J=8.3 Hz), 8.02 (1H, d, J=2.1 Hz).

MS m/z 528 (M+H)⁺.

### Example 207

### [(3S)-6-{[3-(6-methoxypyridin-3-yl)-2-methyl-4-(trifluoromethyl)benzyl]amino}-2,3-dihydro-1-benzofuran-3-yl]acetic acid

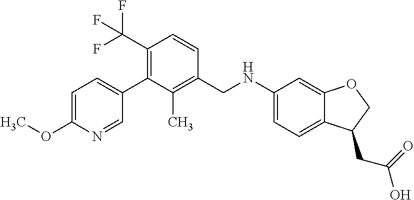

In the same manner as in Example 206, the title compound was obtained as a beige solid from methyl [(3S)-6-{[3-(6-methoxypyridin-3-yl)-2-methyl-4-(trifluoromethyl)benzyl](trifluoroacetyl)amino}-2,3-dihydro-1-benzofuran-3-yl]acetate. yield 74%.

¹H NMR (300 MHz, CDCl₃) δ 2.01 (3H, s), 2.60 (1H, dd, J=17.0, 9.5 Hz), 2.79 (1H, dd, J=17.0, 5.7 Hz), 3.71-3.84 (1H, m), 4.00 (3H, s), 4.25 (1H, dd, J=9.3, 5.9 Hz), 4.33 (2H, s), 4.73 (1H, t, J=9.1 Hz), 6.10 (1H, d, J=1.9 Hz), 6.16 (1H, dd, J=8.0, 1.9 Hz), 6.82 (1H, d, J=8.3 Hz), 6.99 (1H, d, J=8.0 Hz), 7.40 (1H, dd, J=8.7, 2.3 Hz), 7.45-7.52 (1H, m), 7.53-7.61 (1H, m), 7.97 (1H, s).

MS m/z 473 (M+H)⁺.

### Example 208

### methyl [(3S)-6-({2-methyl-3-[2-(trifluoromethyl)imidazo[1,2-a]pyridin-3-yl]benzyl}amino)-2,3-dihydro-1-benzofuran-3-yl]acetate

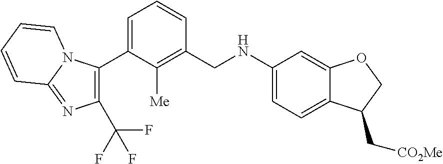

In the same manner as in Example 200, the title compound was obtained as a yellow oil from methyl [(3S)-6-{[2-methyl-3-(4,4,5,5-tetramethyl-1,3,2-dioxaborolan-2-yl)benzyl]amino}-2,3-dihydro-1-benzofuran-3-yl]acetate and 3-bromo-2-(trifluoromethyl)imidazo[1,2-a]pyridine. yield 60%.

¹H NMR (300 MHz, CDCl₃) δ 2.03 (3H, s), 2.55 (1H, dd, J=16.4, 9.2 Hz), 2.75 (1H, dd, J=16.4, 5.5 Hz), 3.68-3.87 (4H, m), 4.02 (1H, br s), 4.24 (1H, dd, J=9.0, 6.0 Hz), 4.34 (2H, s), 4.73 (1H, t, J=8.9 Hz), 6.15 (1H, d, J=1.9 Hz), 6.20 (1H, dd, J=7.9, 2.1 Hz), 6.83-6.91 (1H, m), 6.97 (1H, d, J=8.1 Hz), 7.22-7.39 (3H, m), 7.51-7.60 (2H, m), 7.72-7.78 (1H, m).

MS m/z 496 (M+H)⁺.

### Example 209

### [(3S)-6-({2-methyl-3-[2-(trifluoromethyl)imidazo[1,2-a]pyridin-3-yl]benzyl}amino)-2,3-dihydro-1-benzofuran-3-yl]acetic acid

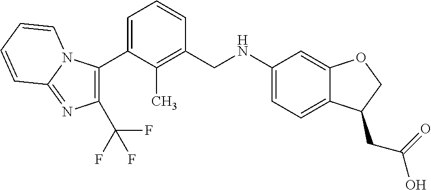

In the same manner as in Example 185, the title compound was obtained as a beige solid from methyl [(3S)-6-({2-methyl-3-[2-(trifluoromethyl)imidazo[1,2-a]pyridin-3-yl]benzyl}amino)-2,3-dihydro-1-benzofuran-3-yl]acetate. yield 78%.

¹H NMR (300 MHz, CDCl₃) δ 2.03 (3H, s), 2.62 (1H, dd, J=16.6, 9.2 Hz), 2.82 (1H, dd, J=16.6, 5.3 Hz), 3.74-3.88 (1H, m), 4.28 (1H, dd, J=9.2, 6.0 Hz), 4.35 (2H, s), 4.75 (1H, t, J=8.9 Hz), 6.12-6.25 (2H, m), 6.84-6.93 (1H, m), 7.01 (1H, d, J=8.1 Hz), 7.22-7.29 (1H, m), 7.29-7.40 (2H, m), 7.50-7.60 (2H, m), 7.76-7.84 (1H, m).

### Example 210

### [(3S)-6-({3-[5-fluoro-6-(morpholin-4-yl)pyridin-3-yl]-2-methyl-4-(trifluoromethyl)benzyl}amino)-2,3-dihydro-1-benzofuran-3-yl]acetic acid

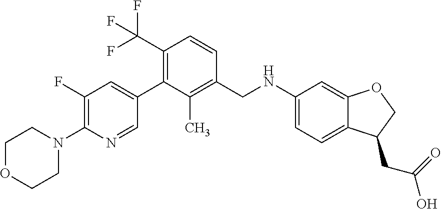

In the same manner as in Example 206, the title compound was obtained as a beige solid from methyl [(3S)-6-{[3-(5-fluoro-6-morpholin-4-ylpyridin-3-yl)-2-methyl-4-(trifluoromethyl)benzyl](trifluoroacetyl)amino}-2,3-dihydro-1-benzofuran-3-yl]acetate. yield 79%.

¹H NMR (300 MHz, CDCl₃) δ 2.04 (3H, s), 2.60 (1H, dd, J=17.0, 9.5 Hz), 2.79 (1H, dd, J=16.7, 5.3 Hz), 3.51-3.61 (4H, m), 3.71-3.94 (5H, m), 4.25 (1H, dd, J=9.3, 5.9 Hz), 4.33 (2H, s), 4.73 (1H, t, J=9.1 Hz), 6.09 (1H, d, J=1.9 Hz), 6.16 (1H, dd, J=8.1, 2.1 Hz), 6.99 (1H, d, J=8.3 Hz), 7.07-7.17 (1H, m), 7.45-7.61 (2H, m), 7.83 (1H, s).

MS m/z 546 (M+H)⁺.

### Example 211

### methyl [(3S)-6-{[3-(5-fluoro-6-morpholin-4-ylpyridin-3-yl)-2,4-dimethylbenzyl]amino}-2,3-dihydro-1-benzofuran-3-yl]acetate

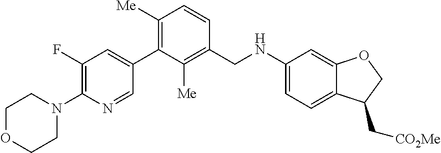

In the same manner as in Example 184, the title compound was obtained as a yellow oil from methyl {(3S)-6-[(3-bromo-2,4-dimethylbenzyl)amino]-2,3-dihydro-1-benzofuran-3-yl}acetate and (5-fluoro-6-morpholin-4-ylpyridin-3-yl)boronic acid. yield 100%.

¹H NMR (300 MHz, CDCl₃) δ 2.04 (6H, s), 2.54 (1H, dd, J=16.6, 9.4 Hz), 2.74 (1H, dd, J=16.2, 5.3 Hz), 3.50-3.58 (4H, m), 3.68-3.93 (9H, m), 4.18-4.2:8 (3H, m), 4.71 (1H, t, J=8.9 Hz), 6.11-6.20 (2H, m), 6.94 (1H, d, J=7.9 Hz), 7.04-7.13 (2H, m), 7.23-7.29 (1H, m), 7.81 (1H, t, J=1.5 Hz).

MS m/z 506 (M+H)⁺.

### Example 212

### [(3S)-6-({3-[5-fluoro-6-(morpholin-4-yl)pyridin-3-yl]-2,4-dimethylbenzyl}amino)-2,3-dihydro-1-benzofuran-3-yl]acetic acid

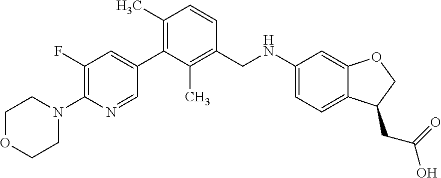

In the same manner as in Example 185, the title compound was obtained as a yellow oil from methyl [(3S)-6-{[3-(5-fluoro-6-morpholin-4-ylpyridin-3-yl)-2,4-dimethylbenzyl]amino}-2,3-dihydro-1-benzofuran-3-yl]acetate. yield 78%.

¹H NMR (300 MHz, CDCl₃) δ 2.05 (6H, s), 2.59 (1H, dd, J=16.7, 9.5 Hz), 2.79 (1H, dd, J=17.0, 5.3 Hz), 3.49-3.59 (4H, m), 3.71-3.94 (5H, m), 4.19-4.30 (3H, m), 4.72 (1H, t, J=9.1 Hz), 6.11-6.22 (2H, m), 6.98 (1H, d, J=8.3 Hz), 7.04-7.14 (2H, m), 7.22-7.29 (1H, m), 7.81 (1H, t, J=1.5 Hz).

MS m/z 492 (M+H)⁺.

### Example 213

### sodium [(3S)-6-({3-[5-fluoro-6-(morpholin-4-yl)pyridin-3-yl]-2,4-dimethylbenzyl}amino)-2,3-dihydro-1-benzofuran-3-yl]acetate

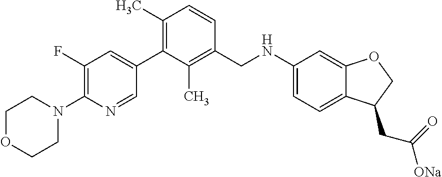

To a suspension of [(3S)-6-({3-[5-fluoro-6-(morpholin-4-yl)pyridin-3-yl]-2,4-dimethylbenzyl}amino)-2,3-dihydro-1-benzofuran-3-yl]acetic acid (0.177 g, 0.359 mmol) in water (2 mL) was added 1 M aqueous sodium hydroxide solution (0.359 mL), and the mixture was stirred at room temperature. After dissolution of insoluble material, acetonitrile was added and the mixture was concentrated under reduced pressure to give the title compound (0.174 g, yield 94%) as a white solid.

¹H NMR (300 MHz, DMSO-d₆) δ 1.90-2.15 (7H, m), 2.31 (1H, dd, J=15.1, 4.9 Hz), 3.38-3.60 (5H, m), 3.69-3.81 (4H, m), 4.04 (1H, dd, J=8.9, 7.0 Hz), 4.13 (2H, d, J=5.7 Hz), 4.57 (1H, t, J=8.9 Hz), 5.85 (1H, t), 5.96 (1H, d, J=1.9 Hz), 6.06 (1H, dd, J=8.1; 2.1 Hz), 6.86 (1H, d, J=8.3 Hz), 7.08 (1H, d, J=7.5 Hz), 7.21 (1H, d, J=7.9 Hz), 7.41 (1H, dd, J=13.9, 1.9 Hz), 7.81 (1H, t, J=1.5 Hz).

MS m/z 492 (M+H)⁺ (as free form).

### Example 214

### methyl [(3S)-6-{[2,4-dimethyl-3-(5-methyl-6-morpholin-4-ylpyridin-3-yl)benzyl]amino}-2,3-dihydro-1-benzofuran-3-yl]acetate

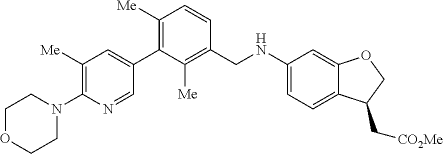

In the same manner as in Example 184, the title compound was obtained as a yellow oil from methyl {(3S)-6-[(3-bromo-2,4-dimethylbenzyl)amino]-2,3-dihydro-1-benzofuran-3-yl}acetate and (5-methyl-6-morpholin-4-ylpyridin-3-yl)boronic acid. yield 98%.

¹H NMR (300 MHz, CDCl₃) δ 2.02 (6H, s), 2.33 (3H, s), 2.54 (1H, dd, J=16.6, 9.4 Hz), 2.74 (1H, dd, J=16.2, 5.3 Hz), 3.23 (4H, d, J=4.9 Hz), 3.68-3.83 (4H, m), 3.83-3.94 (5H, m), 4.17-4.28 (3H, m), 4.71 (1H, t, J=8.9 Hz), 6.10-6.21 (2H, m), 6.94 (1H, d, J=7.9 Hz), 7.09 (1H, d, J=7.9 Hz), 7.20-7.24 (2H, m), 7.96 (1H, d, J=2.3 Hz).

MS m/z 502 (M+H)⁺.

### Example 215

### [(3S)-6-({2,4-dimethyl-3-[5-methyl-6-(morpholin-4-yl)pyridin-3-yl]benzyl}amino)-2,3-dihydro-1-benzofuran-3-yl]acetic acid

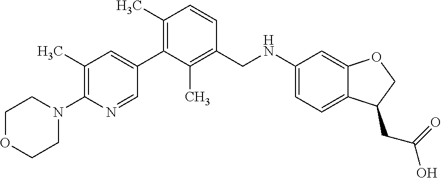

In the same manner as in Example 185, the title compound was obtained as a beige solid from methyl [(3S)-6-{[2,4-dimethyl-3-(5-methyl-6-morpholin-4-ylpyridin-3-yl)benzyl]amino}-2,3-dihydro-1-benzofuran-3-yl]acetate. yield 93%.

¹H NMR (300 MHz, CDCl₃) δ 2.02 (6H, s), 2.33 (3H, s), 2.59 (1H, dd, J=16.7, 9.1 Hz), 2.79 (1H, dd, J=16.7, 5.3 Hz), 3.17-3.28 (4H, m), 3.71-3.85 (1H, m), 3.84-3.96 (4H, m), 4.17-4.32 (3H, m), 4.73 (1H, t, J=8.9 Hz), 6.10-6.23 (2H, m), 6.98 (1H, d, J=8.0 Hz), 7.09 (1H, d, J=8.0 Hz), 7.18-7.29 (2H, m), 7.96 (1H, d, J=1.9 Hz).

MS m/z 488 (M+H)⁺.

### Example 216

### methyl [(3S)-6-({2-methyl-3-[2-(trifluoromethyl)imidazo[1,2-a]pyridin-6-yl]benzyl}amino)-2,3-dihydro-1-benzofuran-3-yl]acetate

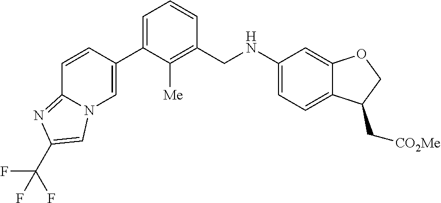

A solution of 6-bromo-2-(trifluoromethyl)imidazo[1,2-a]pyridine (0.300 g, 1.13 mmol), bis(pinacolato)diboron (0.431 g, 1.70 mmol) and potassium acetate (0.389 g, 3.96 mmol) in N,N-dimethylformamide (6 mL) was substituted with argon, and [1,1-bis(diphenylphosphino)ferrocene]dichloropalladium (II) (25.1 mg, 0.0340 mmol) was added. The reaction mixture was stirred under an argon atmosphere at 100° C. for 4 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=90:10-50:50) to give 6-(4,4,5,5-tetramethyl-1,3,2-dioxaborolan-2-yl)-2-(trifluoromethyl)imidazo[1,2-a]pyridine (0.186 g, yield 53%) as a colorless oil. Then, methyl {(3S)-6-[(3-bromo-2-methylbenzyl)amino]-2,3-dihydro-1-benzofuran-3-yl}acetate (96.0 mg, 0.245 mmol) and 6-(4,4,5,5-tetramethyl-1,3,2-dioxaborolan-2-yl)-2-(trifluoromethyl)imidazo[1,2-a]pyridine (88.1 mg, 0.282 mmol) obtained above were suspended in a mixed solution of 2 M aqueous sodium carbonate solution (0.295 mL) and toluene (3 mL) and, after argon substitution, tris(dibenzylideneacetone)dipalladium (0) (8.9 mg, 9.82 μmol) and 2-dicyclohexylphosphino-2′,6′-dimethoxybiphenyl (16.6 mg, 0.0390 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=95:5-60:40) to give the title compound (97.4 mg, yield 80%) as a colorless oil.

MS m/z 496 (M+H)⁺.

### Example 217

### [(3S)-6-({2-methyl-3-[2-(trifluoromethyl)imidazo[1,2-a]pyridin-6-yl]benzyl}amino)-2,3-dihydro-1-benzofuran-3-yl]acetic acid

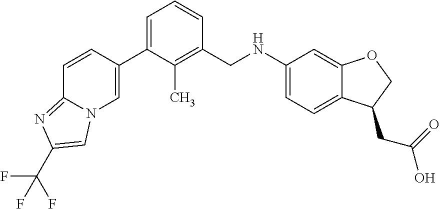

In the same manner as in Example 185, the title compound was obtained as a white solid from methyl [(3S)-6-({2-methyl-3-[2-(trifluoromethyl)imidazo[1,2-a]pyridin-6-yl]benzyl}amino)-2,3-dihydro-1-benzofuran-3-yl]acetate. yield 81%.

¹H NMR (300 MHz, CDCl₃) δ 2.25 (3H, s), 2.61 (1H, dd, J=17.0, 9.5 Hz), 2.81 (1H, dd, J=16.7, 5.3 Hz), 3.72-3.87 (1H, m), 4.21-4.37 (3H, m), 4.74 (1H, t, J=8.9 Hz), 6.11-6.24 (2H, m), 7.00 (1H, d, J=8.3 Hz), 7.15-7.35 (3H, m), 7.44 (1H, dd, J=7.6, 1.1 Hz), 7.75 (1H, d, J=9.1 Hz), 7.92 (1H, s), 8.07 (1H, d, J=1.1 Hz).

MS m/z 482 (M+H)⁺.

### Example 218

### methyl [(3S)-6-({2,4-dimethyl-3-[2-(trifluoromethyl)imidazo[1,2-a]pyridin-6-yl]benzyl}amino)-2,3-dihydro-1-benzofuran-3-yl]acetate

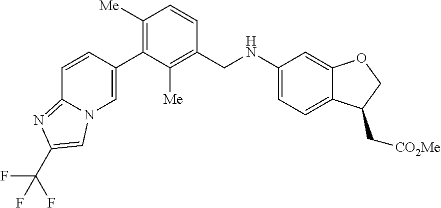

In the same manner as in Example 184, the title compound was obtained as a colorless oil from methyl {(3S)-6-[(3-bromo-2,4-dimethylbenzyl)amino]-2,3-dihydro-1-benzofuran-3-yl}acetate and 6-(4,4,5,5-tetramethyl-1,3,2-dioxaborolan-2-yl)-2-(trifluoromethyl)imidazo[1,2-a]pyridine obtained in the process of Example 216. yield 73%.

MS m/z 510 (M+H)⁺.

### Example 219

### [(3S)-6-({2,4-dimethyl-3-[2-(trifluoromethyl)imidazo[1,2-a]pyridin-6-yl]benzyl}amino)-2,3-dihydro-1-benzofuran-3-yl]acetic acid

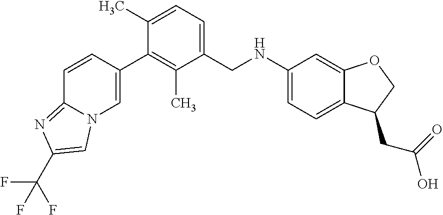

To a mixed solution of methyl [(3S)-6-({2,4-dimethyl-3-[2-(trifluoromethyl)imidazo[1,2-a]pyridin-6-yl]benzyl}amino)-2,3-dihydro-1-benzofuran-3-yl]acetate (99.0 mg, 0.194 mmol) in tetrahydrofuran (2 mL) and methanol (1 mL) was added 1 M aqueous sodium hydroxide solution (0.583 mL), and the mixture was stirred at 50° C. for 30 min. The reaction mixture was concentrated under reduced pressure, water was added to dissolve the residue, and 1 M hydrochloric acid (0.583 mL) was slowly added. The obtained precipitate was collected by filtration, washed with water, and dried to give the title compound (81.8 mg, yield 85%) as a white solid.

¹H NMR (300 MHz, CDCl₃) δ 2.06 (3H, s), 2.07 (3H, s), 2.60 (1H, dd, J=16.7, 9.1 Hz), 2.81 (1H, dd, J=16.7, 5.3 Hz), 3.72-3.86 (1H, m), 4.20-4.32 (3H, m), 4.74 (1H, t, J=8.9 Hz), 6.10-6.22 (2H, m), 6.99 (1H, d, J=8.0 Hz), 7.09-7.18 (2H, m), 7.32 (1H, d, J=8.0 Hz), 7.81 (1H, d, J=9.5 Hz), 7.89-7.97 (2H, m).

MS m/z 496 (M+H)⁺.

### Example 220

### methyl [(3S)-6-{[2-methyl-3-(2-methylfuro[3,2-b]pyridin-3-yl)benzyl]amino}-2,3-dihydro-1-benzofuran-3-yl]acetate

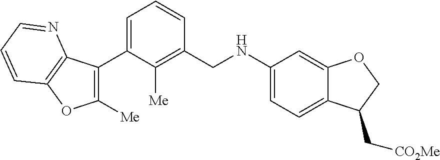

In the same manner as in Example 184, the title compound was obtained as a white solid from methyl [(3S)-6-{[2-methyl-3-(4,4,5,5-tetramethyl-1,3,2-dioxaborolan-2-yl)benzyl]amino}-2,3-dihydro-1-benzofuran-3-yl]acetate and 2-methylfuro[3,2-b]pyridin-3-yl trifluoromethanesulfonate. yield 68%.

¹H NMR (300 MHz, CDCl₃) δ 2.21 (3H, s), 2.43 (3H, s), 2.54 (1H, dd, J=16.3, 9.1 Hz), 2.75 (1H, dd, J=16.7, 5.7 Hz), 3.68-3.85 (4H, m), 3.98 (1H, br s), 4.23 (1H, dd, J=9.1, 6.1 Hz), 4.30 (2H, s), 4.72 (1H, t, J=8.9 Hz), 6.14-6.24 (2H, m), 6.95 (1H, d, J=8.0 Hz), 7.18 (1H, dd, J=8.1, 4.7 Hz), 7.21-7.30 (2H, m), 7.38 (1H, dd, J=6.2, 2.5 Hz), 7.71 (1H, dd, J=8.3, 1.1 Hz), 8.48 (1H, dd, J=4.9, 1.5 Hz).

MS m/z 443 (M+H)⁺.

### Example 221

### [(3S)-6-{[2-methyl-3-(2-methylfuro[3,2-b]pyridin-3-yl)benzyl]amino}-2,3-dihydro-1-benzofuran-3-yl]acetic acid

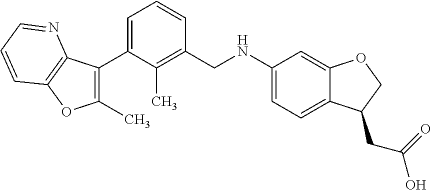

In the same manner as in Example 219, the title compound was obtained as a pale-yellow non-crystalline powder from methyl [(3S)-6-{[2-methyl-3-(2-methylfuro[3,2-b]pyridin-3-yl)benzyl]amino}-2,3-dihydro-1-benzofuran-3-yl]acetate. yield 85%.

¹H NMR (300 MHz, CDCl₃) δ 2.17 (3H, s), 2.36-2.52 (4H, m), 2.64-2.75 (1H, m), 3.61-3.77 (1H, m), 4.12-4.22 (1H, m), 4.23-4.42 (2H, m), 4.60-4.75 (1H, m), 6.08-6.21 (2H, m), 6.87 (1H, t, J=8.3 Hz), 7.15-7.29 (3H, m), 7.40 (1H, d), 7.75-7.81 (1H, m), 8.54-8.61 (1H, m).

MS m/z 429 (M+H)⁺.

### Example 222

### methyl [(3S)-6-{[2-methyl-3-(2-methylfuro[2,3-b]pyridin-3-yl)benzyl]amino}-2,3-dihydro-1-benzofuran-3-yl]acetate

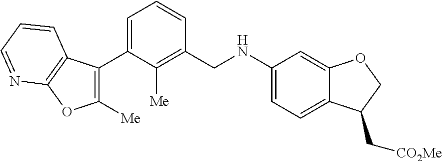

A solution of 3-bromo-2-methylfuro[2,3-b]pyridine (0.330 g, 1.56 mmol), bis(pinacolato)diboron (0.593 g, 2.33 mmol) and potassium acetate (0.534 g, 5.45 mmol) in N,N-dimethylformamide (6 mL) was substituted with argon, and [1,1-bis(diphenylphosphino) ferrocene]dichloropalladium (II) (34.4 mg, 0.0470 mmol) was added. The reaction mixture was stirred under an argon atmosphere at 100° C. for 5 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=100:0-80:20) to give a mixture (0.157 g, a white solid) of 2-methyl-3-(4,4,5,5-tetramethyl-1,3,2-dioxaborolan-2-yl)furo[2,3-b]pyridine and the starting material 3-bromo-2-methylfuro[2,3-b]pyridine. Then, a mixture (0.157 g) of methyl {(3S)-6-[(3-bromo-2-methylbenzyl)amino]-2,3-dihydro-1-benzofuran-3-yl}acetate (0.216 g, 0.552 mmol), 2-methyl-3-(4,4,5,5-tetramethyl-1,3,2-dioxaborolan-2-yl)furo[2,3-b]pyridine obtained above and 3-bromo-2-methylfuro[2,3-b]pyridine was suspended in a mixed solution of 2 M aqueous sodium carbonate solution (0.663 mL) and toluene (3 mL) and, after argon substitution, tris(dibenzylideneacetone)dipalladium (0) (20.2 mg, 0.0220 mmol) and 2-dicyclohexylphosphino-2′,6′-dimethoxybiphenyl (37.4 mg, 0.0880 mmol) were added. The reaction mixture was stirred under an argon atmosphere at 100° C. for 16 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=100:0-70:30) to give the title compound (0.121 g, yield 49%) as a white non-crystalline powder.

¹H NMR (300 MHz, CDCl₃) δ 2.18 (3H, s), 2.40 (3H, s), 2.55 (1H, dd, J=16.3, 9.1 Hz), 2.75 (1H, dd, J=16.3, 5.7 Hz), 3.71 (4H, s), 3.98 (1H, br s), 4.23 (1H, dd, J=9.5, 6.1 Hz), 4.32 (2H, s), 4.72 (1H, t, J=8.9 Hz), 6.12-6.23 (2H, m), 6.96 (1H, d, J=8.0 Hz), 7.13-7.31 (3H, m), 7.37-7.44 (1H, m), 7.56 (1H, dd, J=7.6, 1.5 Hz), 8.27 (1H, dd, J=4.9, 1.5 Hz).

MS m/z 443 (M+H)⁺.

### Example 223

### [(3S)-6-{[2-methyl-3-(2-methylfuro[2,3-b]pyridin-3-yl)benzyl]amino}-2,3-dihydro-1-benzofuran-3-yl]acetic acid

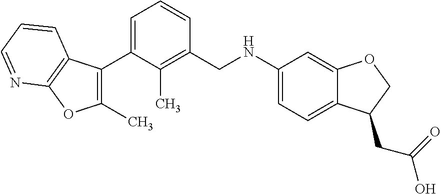

In the same manner as in Example 185, the title compound was obtained as a white solid from methyl [(3S)-6-{[2-methyl-3-(2-methylfuro[2,3-b]pyridin-3-yl)benzyl]amino}-2,3-dihydro-1-benzofuran-3-yl]acetate. yield 82%.

¹H NMR (300 MHz, CDCl₃) δ 2.18 (3H, s), 2.40 (3H, s), 2.61 (1H, dd, J=16.7, 9.1 Hz), 2.81 (1H, dd, J=16.7, 5.3 Hz), 3.72-3.88 (1H, m), 4.26 (1H, dd, J=9.1, 6.1 Hz), 4.32 (2H, s), 4.74 (1H, t, J=8.9 Hz), 6.12-6.25 (2H, m), 7.00 (1H, d, J=8.0 Hz), 7.13-7.31 (2H, m), 7.38-7.44 (2H, m), 7.57 (1H, dd, J=7.6, 1.5 Hz), 8.27 (1H, dd, J=4.7, 1.3 Hz).

MS m/z 429 (M+H)⁺.

### Example 224

### [(3S)-6-{[4-(6-fluoro-2-methyl-1H-benzimidazol-1-yl)-2,3-dihydro-1H-inden-1-yl]amino}-2,3-dihydro-1-benzofuran-3-yl]acetic acid

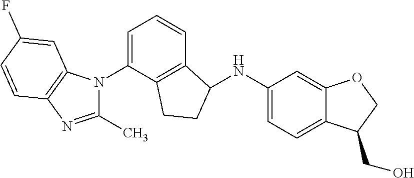

In the same manner as in Example 206, the title compound was obtained as a white solid from methyl [(3S)-6-{[4-(6-fluoro-2-methyl-1H-benzimidazol-1-yl)-2,3-dihydro-1H-inden-1-yl](trifluoroacetyl)amino}-2,3-dihydro-1-benzofuran-3-yl]acetate. yield 95%.

¹H NMR (300 MHz, CDCl₃) δ 1.81-1.98 (1H, m), 2.44 (3H, d, J=4.5 Hz), 2.53-2.71 (4H, m), 2.83 (1H, dd, J=16.7, 5.3 Hz), 3.75-3.91 (1H, m), 4.31 (1H, dd, J=8.9, 6.2 Hz), 4.78 (1H, t, J=9.1 Hz), 5.06-5.17 (1H, m), 6.22-6.32 (2H, m), 6.59-6.70 (1H, m), 6.96-7.08 (2H, m), 7.20 (1H, d, J=7.6 Hz), 7.43 (1H, t, J=7.8 Hz), 7.57 (1H, d, J=7.6 Hz), 7.64-7.75 (1H, m).

MS m/z 458 (M+H)⁺.

### Example 225

### [(3S)-6-{[4-(2-ethyl-6-fluoro-1H-benzimidazol-1-yl)-2,3-dihydro-1H-inden-1-yl]amino}-2,3-dihydro-1-benzofuran-3-yl]acetic acid

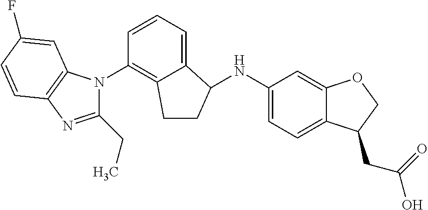

In the same manner as in Example 206, the title compound was obtained as a white solid from methyl [(3S)-6-{[4-(2-ethyl-6-fluoro-1H-benzimidazol-1-yl)-2,3-dihydro-1H-inden-1-yl](trifluoroacetyl)amino}-2,3-dihydro-1-benzofuran-3-yl]acetate. yield 100%.

¹H NMR (300 MHz, CDCl₃) δ 1.32 (3H, t, J=7.6 Hz), 1.79-1.99 (1H, m), 2.45-2.92 (7H, m), 3.77-3.92 (1H, m), 4.32 (1H, dd, J=9.5, 6.1 Hz), 4.79 (1H, t, J=8.9 Hz), 5.06-5.17 (1H, m), 6.22-6.32 (2H, m), 6.58-6.68 (1H, m), 6.96-7.09 (2H, m), 7.20 (1H, d, J=7.6 Hz), 7.42 (1H, t, J=7.6 Hz), 7.58 (1H, d, J=7.6 Hz), 7.70-7.81 (1H, m).

MS m/z 472 (M+H)⁺.

### Example 226

### [(3S)-6-{[4-(2-ethoxy-6-fluoro-1H-benzimidazol-1-yl)-2,3-dihydro-1H-inden-1-yl]amino}-2,3-dihydro-1-benzofuran-3-yl]acetic acid

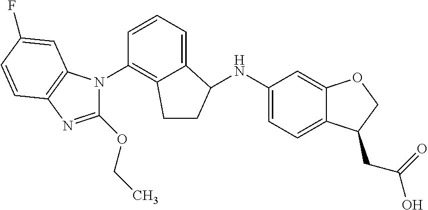

In the same manner as in Example 206, the title compound was obtained as a white solid from methyl [(3S)-6-{[4-(2-ethoxy-6-fluoro-1H-benzimidazol-1-yl)-2,3-dihydro-1H-inden-1-yl](trifluoroacetyl)amino}-2,3-dihydro-1-benzofuran-3-yl]acetate. yield 98%.

¹H NMR (300 MHz, CDCl₃) δ 1.43 (3H, td, J=7.0, 4.2 Hz), 1.76-2.02 (1H, m), 2.50-2.92 (5H, m), 3.75-3.89 (1H, m), 4.29 (1H, dd, J=9.1, 6.1 Hz), 4.52-4.71 (2H, m), 4.76 (1H, t, J=8.9 Hz), 5.03-5.15 (1H, m), 6.22-6.32 (2H, m), 6.60-6.73 (1H, m), 6.88-6.99 (1H, m), 7.02 (1H, d, J=8.3 Hz), 7.22-7.28 (1H, m), 7.34-7.43 (1H, m), 7.51 (2H, dd, J=8.9, 4.7 Hz).

MS m/z 488 (M+H)⁺.

### Example 227

### sodium [(3S)-6-{[4-(2-ethoxy-6-fluoro-1H-benzimidazol-1-yl)-2,3-dihydro-1H-inden-1-yl]amino}-2,3-dihydro-1-benzofuran-3-yl]acetate

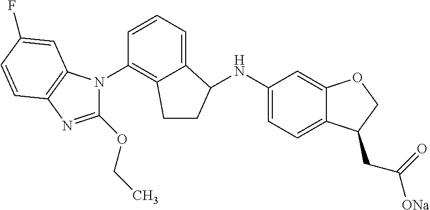

In the same manner as in Example 213, the title compound was obtained as a pale-yellow solid from [(3S)-6-{[4-(2-ethoxy-6-fluoro-1H-benzimidazol-1-yl)-2,3-dihydro-1H-inden-1-yl]amino}-2,3-dihydro-1-benzofuran-3-yl]acetic acid. yield 98%.

¹H NMR (300 MHz, DMSO-d₆) δ 1.28-1.41 (3H, m), 1.69-1.88 (1H, m), 1.94-2.08 (1H, m), 2.27-2.78 (4H, m), 3.47-3.66 (1H, m), 4.02-4.15 (1H, m), 4.46-4.70 (3H, m), 4.97-5.14 (1H, m), 5.77-5.97 (1H, m), 6.11-6.29 (2H, m), 6.69-6.81 (1H, m), 6.87-7.08 (2H, m), 7.27-7.57 (4H, m).

MS m/z 488 (M+H)⁺ (as free form).

### Example 228

### methyl [(3S)-6-{[3-(2-ethyl-7-methyl-1H-benzimidazol-1-yl)-2-methylbenzyl]amino}-2,3-dihydro-1-benzofuran-3-yl]acetate

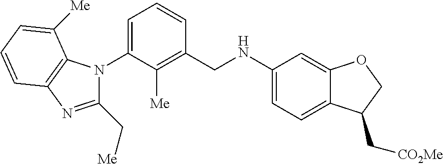

A solution of methyl 3-(2-ethyl-7-methyl-1H-benzimidazol-1-yl)-2-methylbenzoate (0.410 g, 1.33 mmol) in tetrahydrofuran (5 mL) was cooled to 0° C., and lithium aluminum hydride (50.5 mg, 1.33 mmol) was slowly added. The reaction mixture was stirred at 0° C. for 30 min, and sodium sulfate 10 hydrate was slowly added at the same temperature. The reaction mixture was further stirred for 20 min, and the insoluble material was filtered off. The filtrate was concentrated under reduced pressure to give a crude product (0.481 g) of [3-(2-ethyl-7-methyl-1H-benzimidazol-1-yl)-2-methylphenyl]methanol as a pale-yellow oil. To a solution of [3-(2-ethyl-7-methyl-1H-benzimidazol-1-yl)-2-methylphenyl]methanol (0.481 g) obtained above in acetonitrile (7 mL) was slowly added a Dess-Martin reagent (0.875 g, 2.06 mmol) at 0° C. The reaction mixture was stirred at room temperature for 40 min, aqueous sodium bicarbonate solution and saturated aqueous sodium thiosulfate solution were added, and the mixture was further stirred for min. The reaction mixture was extracted with ethyl acetate, and the obtained extract was dried over sodium sulfate and concentrated under reduced pressure to give a crude product (0.452 g) of 3-(2-ethyl-7-methyl-1H-benzimidazol-1-yl)-2-methylbenzaldehyde as a yellow oil. 3-(2-Ethyl-7-methyl-1H-benzimidazol-1-yl)-2-methylbenzaldehyde (0.452 g) obtained above and methyl [(3S)-6-amino-2,3-dihydro-1-benzofuran-3-yl]acetate (0.337 g, 1.62 mmol) were dissolved in acetic acid (0.279 mL, 4.87 mmol) and acetonitrile (10 mL), sodium triacetoxyborohydride (0.688 g, 3.25 mmol) was added at 0° C., and the mixture was stirred at room temperature for 2 hr. The reaction mixture was neutralized with aqueous sodium bicarbonate solution, and the mixture was extracted with ethyl acetate. The extract was dried over sodium sulfate, and concentrated under reduced pressure. The residue was purified by silica gel column chromatography (hexane:ethyl acetate=95:5-50:50) to give the title compound (0.617 g, yield 99% (3 steps) as a pale-yellow non-crystalline powder.

¹H NMR (300 MHz, CDCl₃) δ 1.34 (3H, t, J=7.5 Hz), 1.82 (3H, s), 1.91 (3H, s), 2.48-2.63 (3H, m), 2.74 (1H, dd, J=16.2, 5.7 Hz), 3.68-3.85 (4H, m), 4.00 (1H, br s), 4.23 (1H, dd, J=9.0, 6.0 Hz), 4.34 (2H, s), 4.72 (1H, t, J=9.0 Hz), 6.10-6.21 (2H, m), 6.88-6.99 (2H, m), 7.15 (1H, t, J=7.5 Hz), 7.21-7.28 (1H, m), 7.32 (1H, t, J=7.7 Hz), 7.54 (1H, d, J=6.8 Hz), 7.66 (1H, d, J=7.9 Hz).

MS m/z 470 (M+H)⁺.

### Example 229

### [(3S)-6-{[3-(2-ethyl-7-methyl-1H-benzimidazol-1-yl)-2-methylbenzyl]amino}-2,3-dihydro-1-benzofuran-3-yl]acetic acid

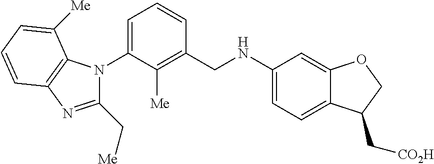

To a mixed solution of methyl [(3S)-6-{[3-(2-ethyl-7-methyl-1H-benzimidazol-1-yl)-2-methylbenzyl]amino}-2,3-dihydro-1-benzofuran-3-yl]acetate (0.617 g, 1.31 mmol) in tetrahydrofuran (4 mL) and methanol (2 mL) was added 1 M aqueous sodium hydroxide solution (3.94 mL), and the mixture was stirred at room temperature for 2 hr. The reaction mixture was neutralized with 1 M hydrochloric acid (3.94 mL) and diluted with brine, and the mixture was extracted with ethyl acetate. The extract was washed with saturated brine, dried over anhydrous sodium sulfate, and concentrated under reduced pressure to give the title compound (0.544 g, yield 91%) as a pale-yellow non-crystalline powder.

¹H NMR (300 MHz, CDCl₃) δ 1.32 (3H, t, J=7.6 Hz), 1.82 (3H, s), 1.92 (3H, s), 2.52-2.69 (3H, m), 2.81 (1H, dd, J=16.7, 5.7 Hz), 3.74-3.90 (1H, m), 4.22-4.40 (3H, m), 4.76 (1H, t, J=8.9 Hz), 6.08-6.21 (2H, m), 6.93 (1H, d, J=7.2 Hz), 7.01 (1H, d, J=8.0 Hz), 7.16 (1H, t, J=7.6 Hz), 7.20-7.27 (1H, m), 7.33 (1H, t, J=7.8 Hz), 7.56 (1H, d, J=7.6 Hz), 7.69 (1H, d, J=8.0 Hz).

MS m/z 456 (M+H)⁺.

### Example 230

### sodium [(3S)-6-{[3-(2-ethyl-7-methyl-1H-benzimidazol-1-yl)-2-methylbenzyl]amino}-2,3-dihydro-1-benzofuran-3-yl]acetate

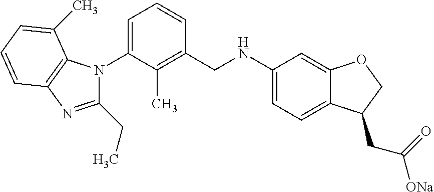

In the same manner as in Example 213, the title compound was obtained as a pale-yellow solid from [(3S)-6-{[3-(2-ethyl-7-methyl-1H-benzimidazol-1-yl)-2-methylbenzyl]amino}-2,3-dihydro-1-benzofuran-3-yl]acetic acid. yield 98%.

¹H NMR (300 MHz, DMSO-d₆) δ 1.22 (3H, t, J=7.5 Hz), 1.76 (3H, s), 1.79 (3H, s), 1.97 (1H, dd, J=15.1, 10.2 Hz), 2.31 (1H, dd, J=14.7, 4.9 Hz), 2.42-2.53 (2H, m), 3.44-3.60 (1H, m), 4.04 (1H, t, J=7.9 Hz), 4.26 (2H, d, J=5.7 Hz), 4.58 (1H, t, J=8.9 Hz), 5.96 (1H, t, J=2.3 Hz), 6.00-6.13 (2H, m), 6.83-6.93 (2H, m), 7.07 (1H, t, J=7.5 Hz), 7.29-7.39 (2H, m), 7.44-7.55 (2H, m).

MS m/z 456 (M+H)(as free form).

### Example 231

### sodium [(3S)-6-{[3-(2-ethyl-6,7-difluoro-1H-benzimidazol-1-yl)-2-methylbenzyl]amino}-2,3-dihydro-1-benzofuran-3-yl]acetate

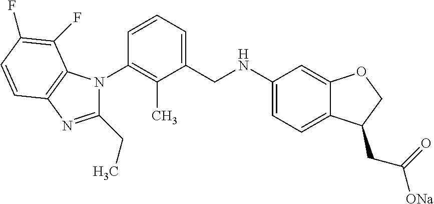

In the same manner as in Example 229, then Example 213, the title compound was obtained as a white solid from methyl [(3S)-6-{[3-(2-ethyl-6,7-difluoro-1H-benzimidazol-1-yl)-2-methylbenzyl](trifluoroacetyl)amino}-2,3-dihydro-1-benzofuran-3-yl]acetate. yield 88% (2 steps).

¹H NMR (300 MHz, DMSO-d₆) δ 1.22 (3H, t, J=7.6 Hz), 1.89 (3H, s), 2.00 (1H, dd, J=15.1, 9.8 Hz), 2.33 (1H, dd, J=15.0, 4.7 Hz), 2.43-2.65 (2H, m), 3.45-3.62 (1H, m), 4.06 (1H, t, J=8.0 Hz), 4.27 (2H, d, J=4.9 Hz), 4.59 (1H, t, J=8.9 Hz), 5.93-6.15 (3H, m), 6.89 (1H, d, J=8.0 Hz), 7.17-7.57 (5H, m).

MS m/z 478 (M+H)⁺ (as free form).

### Example 232

### [(3S)-6-({3-[(5-fluoropyridin-2-yl)(propyl)amino]-2-methylbenzyl}amino)-2,3-dihydro-1-benzofuran-3-yl]acetic acid

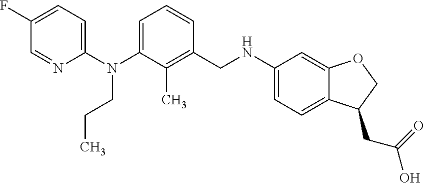

To a solution of methyl {(3S)-6-[{3-[(5-fluoropyridin-2-yl)amino]-2-methylbenzyl}(trifluoroacetyl)amino]-2,3-dihydro-1-benzofuran-3-yl}acetate (0.243 g, 0.469 mmol) and n-propyl iodide (0.119 g, 0.703 mmol) in N,N-dimethylformamide (2 mL) was added sodium hydride (60% in oil, 22.5 mg, 0.562 mmol) at 0° C., and the mixture was stirred at room temperature for 1.5 hr. The reaction mixture was poured into water, and the mixture was extracted with ethyl acetate. The extract was dried over anhydrous sodium sulfate, and concentrated under reduced pressure. The residue was purified by silica gel column chromatography (hexane:ethyl acetate=95:5-70:30) to give a mixture (0.168 g, a pale-yellow oil) of methyl {(3S)-6-[{3-[(5-fluoropyridin-2-yl)(propyl)amino]-2-methylbenzyl}(trifluoroacetyl)amino]-2,3-dihydro-1-benzofuran-3-yl}acetate and methyl [(3S)-6-({3-[(5-fluoropyridin-2-yl)(propyl)amino]-2-methylbenzyl}amino)-2,3-dihydro-1-benzofuran-3-yl]acetate. To a mixed solution of the mixture (0.168 g) obtained above in tetrahydrofuran (2 mL) and methanol (1 mL) was added 1-M aqueous sodium hydroxide solution (1.09 mL), and the mixture was stirred at room temperature for 2 hr. The reaction mixture was neutralized with 1 M hydrochloric acid and diluted with brine, and the mixture was extracted with ethyl acetate. The extract was washed with saturated brine, dried over anhydrous sodium sulfate, and concentrated under reduced pressure to give the title compound (0.137 g, yield 65%, 2 steps) as a pale-yellow non-crystalline powder.

¹H NMR (300 MHz, CDCl₃) δ 0.91 (3H, t, J=7.4 Hz), 1.14-1.34 (2H, m), 1.55-1.78 (2H, m), 2.10 (3H, s), 2.59 (1H, dd, J=16.7, 9.1 Hz), 2.79 (1H, dd, J=16.7, 5.3 Hz), 3.71-3.87 (1H, m), 4.19-4.35 (3H, m), 4.73 (1H, t, J=8.9 Hz), 5.92 (1H, dd, J=9.5, 3.4 Hz), 6.09-6.26 (2H, m), 6.93-7.16 (3H, m), 7.17-7.39 (2H, m), 8.04 (1H, d, J=3.0 Hz).

MS m/z 450 (M+H)⁺.

### Example 233

### methyl {(3S)-6-[(3-bromo-2,4-dimethylbenzyl)amino]-2,3-dihydro-1-benzofuran-3-yl}acetate

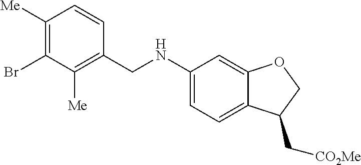

In the same manner as in Example 190, the title compound was obtained as a colorless oil from 3-bromo-2,4-dimethylbenzaldehyde and methyl [(3S)-6-amino-2,3-dihydro-1-benzofuran-3-yl]acetate. yield 77%.

¹H NMR (300 MHz, CDCl₃) δ 2.41 (3H, s), 2.44 (3H, s), 2.53 (1H, dd, J=16.3, 9.1 Hz), 2.73 (1H, dd, J=16.7, 5.7 Hz), 3.68-3.89 (5H, m), 4.18-4.28 (3H, m), 4.71 (1H, t, J=8.9 Hz), 6.08-6.17 (2H, m), 6.93 (1H, d, J=8.0 Hz), 7.05 (1H, d, J=7.6 Hz), 7.16 (1H, d, J=7.6 Hz).

MS m/z 404 (M+H)⁺.

### Example 234

### methyl [(3S)-6-{[3-(4,6-dimethyl-2-morpholin-4-ylpyrimidin-5-yl)-2,4-dimethylbenzyl]amino}-2,3-dihydro-1-benzofuran-3-yl]acetate

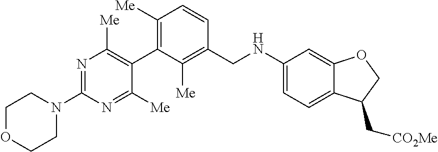

In the same manner as in Example 184, the title compound was obtained as a colorless oil from methyl {(3S)-6-[(3-bromo-2,4-dimethylbenzyl)amino]-2,3-dihydro-1-benzofuran-3-yl}acetate and (4,6-dimethyl-2-morpholin-4-ylpyrimidin-5-yl)boronic acid. yield 8.8%.

¹H NMR (300 MHz, CDCl₃) δ 1.92-2.01 (12H, m), 2.54 (1H, dd, J=16.2, 9.4 Hz), 2.74 (1H, dd, J=16.2, 5.3 Hz), 3.68-3.95 (13H, m), 4.18-4.27 (3H, m), 4.72 (1H, t, J=9.0 Hz), 6.12-6.21 (2H, m), 6.95 (1H, d, J=7.9 Hz), 7.10 (1H, d, J=7.9 Hz), 7.24 (1H, d, J=7.5 Hz).

MS m/z 517 (M+H)⁺.

### Example 235

### [(3S)-6-{[3-(4,6-dimethyl-2-morpholin-4-ylpyrimidin-5-yl)-2,4-dimethylbenzyl]amino}-2,3-dihydro-1-benzofuran-3-yl]acetic acid

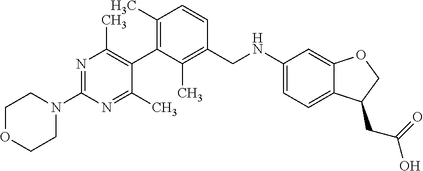

In the same manner as in Example 185, the title compound was obtained as a white solid from methyl [(3S)-6-{[3-(4,6-dimethyl-2-morpholin-4-ylpyrimidin-5-yl)-2,4-dimethylbenzyl]amino}-2,3-dihydro-1-benzofuran-3-yl]acetate. yield 86%.

¹H NMR (300 MHz, CDCl₃) δ 1.92-2.01 (12H, m), 2.60 (1H, dd, J=16.7, 9.1 Hz), 2.80 (1H, dd, J=16.7, 5.3 Hz), 3.71-3.91 (9H, m), 4.21-4.30 (3H, m), 4.73 (1H, t, J=8.9 Hz), 6.13-6.23 (2H, m), 6.98 (1H, d, J=7.6 Hz), 7.10 (1H, d, J=8.0 Hz), 7.21-7.28 (1H, m).

MS m/z 503 (M+H)⁺.

### Example 236

### methyl {(3S)-6-[(3-{5-fluoro-2-methyl-1-[3-(methylsulfonyl)propyl]-1H-indol-3-yl}-2-methylbenzyl)amino]-2,3-dihydro-1-benzofuran-3-yl}acetate

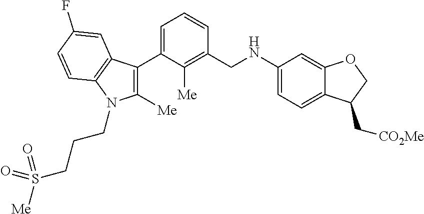

In the same manner as in Example 200, the title compound was obtained as a pale-yellow non-crystalline powder from methyl [(3S)-6-{[2-methyl-3-(4,4,5,5-tetramethyl-1,3,2-dioxaborolan-2-yl)benzyl]amino}-2,3-dihydro-1-benzofuran-3-yl]acetate and 3-bromo-5-fluoro-2-methyl-1-[3-(methylsulfonyl)propyl]-1H-indole. yield 11%.

¹H NMR (300 MHz, CDCl₃) δ 2.12 (3H, s), 2.28 (3H, s), 2.31-2.45 (2H, m), 2.55 (1H, dd, J=16.3, 9.5 Hz), 2.75 (1H, dd, J=16.3, 5.3 Hz), 2.92 (3H, s), 3.05 (2H, t, J=7.4 Hz), 3.69-3.84 (4H, m), 3.96 (1H, br s), 4.23 (1H, dd, J=9.3, 5.9 Hz), 4.28-4.41 (4H, m), 4.72 (1H, t, J=8.9 Hz), 6.15-6.25 (2H, m), 6.86-7.01 (3H, m), 7.15-7.29 (3H, m), 7.32-7.40 (1H, m).

MS m/z 579 (M+H)⁺.

### Example 237

### {(3S)-6-[(3-{5-fluoro-2-methyl-1-[3-(methylsulfonyl)propyl]-1H-indol-3-yl}-2-methylbenzyl)amino]-2,3-dihydro-1-benzofuran-3-yl}acetic acid

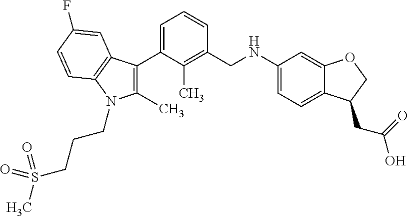

In the same manner as in Example 185, the title compound was obtained as a beige solid from methyl {(3S)-6-[(3-{5-fluoro-2-methyl-1-[3-(methylsulfonyl)propyl]-1H-indol-3-yl}-2-methylbenzyl)amino]-2,3-dihydro-1-benzofuran-3-yl}acetate. yield 84%.

¹H NMR (300 MHz, CDCl₃) δ 2.13 (3H, s), 2.29 (3H, s), 2.31-2.45 (2H, m), 2.61 (1H, dd, J=16.6, 9.0 Hz), 2.81 (1H, dd, J=17.0, 5.3 Hz), 2.92 (3H, s), 3.05 (2H, t, J=7.3 Hz), 3.72-3.86 (1H, m), 4.21-4.41 (5H, m), 4.74 (1H, t, J=8.9 Hz), 6.15-6.25 (2H, m), 6.86-7.04 (3H, m), 7.15-7.30 (3H, m), 7.32-7.40 (1H, m).

MS m/z 565 (M+H)⁺.

### Example 238

### optically active form (A) of {(3S)-6-[(4-bromo-2,3-dihydro-1H-inden-1-yl)amino]-2,3-dihydro-1-benzofuran-3-yl}acetic acid

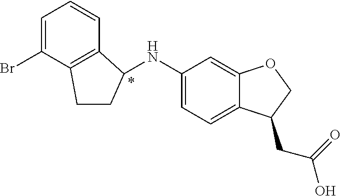

In the same manner as in Example 206, the title compound was obtained as a white solid from an optically active form (A) of methyl {(3S)-6-[(4-bromo-2,3-dihydro-1H-inden-1-yl)(trifluoroacetyl)amino]-2,3-dihydro-1-benzofuran-3-yl}acetate. yield 64%.

¹H NMR (300 MHz, CDCl₃) δ 1.82-1.99 (1H, m), 2.51-2.68 (2H, m), 2.72-2.96 (2H, m), 2.97-3.12 (1H, m), 3.71-3.87 (1H, m), 4.26 (1H, dd, J=9.0, 6.0 Hz), 4.73 (1H, t, J=8.9 Hz), 5.03 (1H, t, J=6.8 Hz), 6.15-6.26 (2H, m), 6.98 (1H, d, J=8.7 Hz), 7.07 (1H, t, J=7.5 Hz), 7.29 (1H, d, J=7.2 Hz), 7.40 (1H, d, J=7.9 Hz).

### Example 239

### optically active form (B) of {(3S)-6-[(4-bromo-2,3-dihydro-1H-inden-1-yl)amino]-2,3-dihydro-1-benzofuran-3-yl}acetic acid

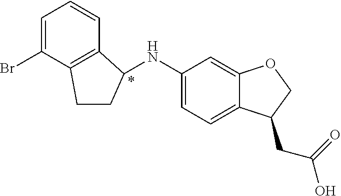

In the same manner as in Example 206, the title compound was obtained as a white solid from an optically active form (B) of methyl {(3S)-6-[(4-bromo-2,3-dihydro-1H-inden-1-yl)(trifluoroacetyl)amino]-2,3-dihydro-1-benzofuran-3-yl}acetate. yield 79%.

¹H NMR (300 MHz, CDCl₃) δ 1.82-2.03 (1H, m), 2.51-2.69 (2H, m), 2.74-2.96 (2H, m), 2.97-3.13 (1H, m), 3.71-3.88 (1H, m), 4.27 (1H, dd, J=9.1, 6.1 Hz), 4.74 (1H, t, J=9.1 Hz), 5.04 (1H, t, J=6.8 Hz), 6.16-6.27 (2H, m), 6.99 (1H, d, J=8.7 Hz), 7.08 (1H, t, J=7.8 Hz), 7.29 (1H, d, J=7.2 Hz), 7.41 (1H, d, J=8.0 Hz).

MS m/z 388 (M+H)⁺.

### Example 240

### [(3S)-6-({3-[4,6-dimethyl-2-(morpholin-4-yl)pyrimidin-5-yl]-2-methylbenzyl}amino)-2,3-dihydro-1-benzofuran-3-yl]acetic acid

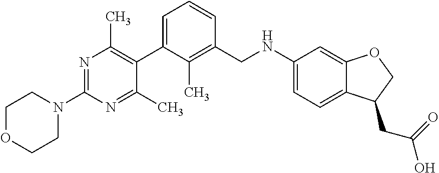

To a mixed solution of methyl [(3S)-6-{[3-(4,6-dimethyl-2-morpholin-4-ylpyrimidin-5-yl)-2-methylbenzyl](trifluoroacetyl)amino}-2,3-dihydro-1-benzofuran-3-yl]acetate (3.63 g, 6.07 mmol) in tetrahydrofuran (30 mL) and methanol (15 mL) was added 1 M aqueous sodium hydroxide solution (18.2 mL), and the mixture was stirred at 50° C. for 2 hr. The reaction mixture was neutralized with 1 M hydrochloric acid and diluted with brine, and the mixture was extracted with ethyl acetate. The extract was washed with saturated brine, dried over anhydrous sodium sulfate, and concentrated under reduced pressure. The residue was crystallized from methanol to give the title compound (2.76 g, yield 93%) as a white solid.

MS m/z 489 (M+H)⁺.

¹H NMR (300 MHz, CDCl₃) δ 1.99-2.07 (9H, m), 2.60 (1H, dd, J=16.6, 9.4 Hz), 2.80 (1H, dd, J=17.0, 5.7 Hz), 3.71-3.90 (9H, m), 4.21-4.33 (3H, m), 4.73 (1H, t, J=9.0 Hz), 6.12-6.23 (2H, m), 6.93-7.03 (2H, m), 7.21 (1H, t, J=7.5 Hz), 7.34 (1H, d, J=6.8 Hz).

### Example 241

### sodium [(3S)-6-({3-[4,6-dimethyl-2-(morpholin-4-yl)pyrimidin-5-yl]-2-methylbenzyl}amino)-2,3-dihydro-1-benzofuran-3-yl]acetate

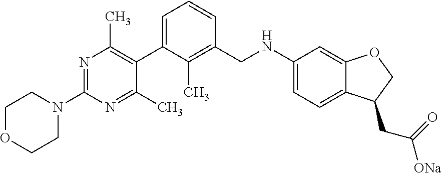

To a solution of [(3S)-6-({3-[4,6-dimethyl-2-(morpholin-4-yl)pyrimidin-5-yl]-2-methylbenzyl}amino)-2,3-dihydro-1-benzofuran-3-yl]acetic acid (51.4 mg, 0.105 mmol) in methanol (1 mL) was added 1 M aqueous sodium hydroxide solution (0.105 mL), and the mixture was stirred at room temperature for 1 min. Acetonitrile was added and the mixture was concentrated under reduced pressure to give the title compound (52.2 mg, yield 97%) as a beige solid.

¹H NMR (300 MHz, DMSO-d₆) δ 1.88-2.04 (10H, m), 2.31 (1H, dd, J=15.1, 4.9 Hz), 3.44-3.60 (1H, m), 3.61-3.79 (8H, m), 4.04 (1H, dd, J=8.7, 7.5 Hz), 4.20 (2H, d, J=5.3 Hz), 4.58 (1H, t, J=8.9 Hz), 5.91 (1H, t, J=5.7 Hz), 5.98 (1H, d, J=1.5 Hz), 6.08 (1H, dd, J=8.3, 1.9 Hz), 6.83-6.99 (2H, m), 7.20 (1H, t, J=7.5 Hz), 7.26-7.34 (1H, m).

MS m/z 489 (M+H)⁺ (as free form).

### Example 242

### 1,3-dihydroxy-2-(hydroxymethyl)propan-2-aminium [(3S)-6-({3[4,6-dimethyl-2-(morpholin-4-yl)pyrimidin-5-yl]-2-methylbenzyl}amino)-2,3-dihydro-1-benzofuran-3-yl]acetate

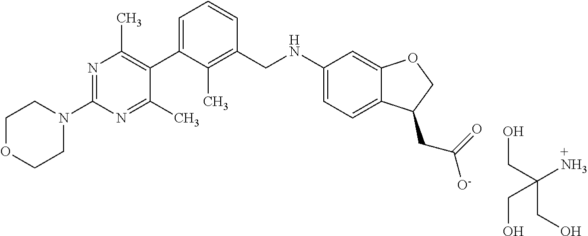

To a solution of [(3S)-6-({3-[4,6-dimethyl-2-(morpholin-4-yl)pyrimidin-5-yl]-2-methylbenzyl}amino)-2,3-dihydro-1-benzofuran-3-yl]acetic acid (2.29 g, 4.69 mmol) in methanol (20 mL) was added 2-amino-2-(hydroxymethyl)propane-1,3-diol (0.568 g, 4.69 mmol), and the mixture was stirred at 50° C. for 5 min. The reaction mixture was concentrated under reduced pressure, ethyl acetate was added, and the mixture was stirred for 16 hr. The resulting crystals were collected by filtration, washed with ethyl acetate, and dried under reduced pressure at 70° C. for 4 days to give the title compound (2.49 g, yield 87%) as white crystals.

¹H NMR (300 MHz, DMSO-d₆) δ 1.89-2.01 (9H, m), 2.22 (1H, dd, J=15.9, 9.5 Hz), 2.43-2.55 (1H, m), 3.33 (6H, s), 3.47-3.78 (9H, m), 4.00-4.10 (1H, m), 4.21 (2H, d, J=3.4 Hz), 4.57 (1H, t, J=8.9 Hz), 5.93-6.04 (2H, m), 6.10 (1H, dd, J=8.1, 1.7 Hz), 6.89 (1H, d, J=8.0 Hz), 6.95 (1H, d, J=6.8 Hz), 7.20 (1H, t, J=7.6 Hz), 7.29 (1H, d, J=7.2 Hz).

MS m/z 489 (M+H)⁺ (as free form).

melting point: 154° C.

elemental analysis value for C₃₂H₄₃N₅O₇.0.5H₂O

Calculated: C, 62.12; H, 7.17; N, 11.32.

Found: C, 61.95; H, 7.18; N, 11.05.

### Example 243

### optically active form of {(3S)-6-[(4-{2,6-dimethyl-4-[3-(methylsulfonyl)propoxy]phenyl}-2,3-dihydro-1H-inden-1-yl)amino]-2,3-dihydro-1-benzofuran-3-yl}acetic acid

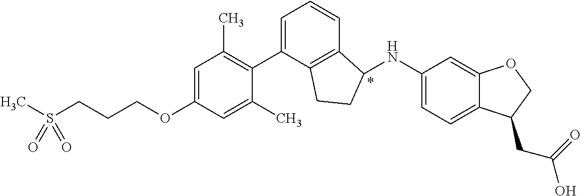

An optically active form (A) (0.194 g, 0.389 mmol) of methyl {(3S)-6-[(4-bromo-2,3-dihydro-1H-inden-1-yl)(trifluoroacetyl)amino]-2,3-dihydro-1-benzofuran-3-yl}acetate and {2,6-dimethyl-4-[3-(methylthio)propoxy]phenyl}boronic acid (0.119 mg, 0.467 mmol) were suspended in a mixed solution of 2 M aqueous sodium carbonate solution (0.467 mL) and toluene (5 mL) and, after argon substitution, tris(dibenzylideneacetone)dipalladium (0) (14.3 mg, 0.0160 mmol) and 2-dicyclohexylphosphino-2′,6′-dimethoxybiphenyl (26.3 mg, 0.0620 mmol) were added. The reaction mixture was stirred under an argon atmosphere at 100° C. for 6 hr. The reaction mixture was cooled, water was added and the mixture was extracted with ethyl acetate. The extract was dried over sodium sulfate, and concentrated under reduced pressure. The residue was purified by silica gel column chromatography (hexane:ethyl acetate=100:0-75:25) to give methyl {(3S)-6-[(4-{2,6-dimethyl-4-[3-(methylthio)propoxy]phenyl}-2,3-dihydro-1H-inden-1-yl)(trifluoroacetyl)amino]-2,3-dihydro-1-benzofuran-3-yl}acetate (99.2 mg, yield 41%) as a colorless oil. To a solution of methyl {(3S)-6-[(4-{2,6-dimethyl-4-[3-(methylthio)propoxy]phenyl}-2,3-dihydro-1H-inden-1-yl)(trifluoroacetyl)amino]-2,3-dihydro-1-benzofuran-3-yl}acetate (99.2 mg, 0.158 mmol) obtained above in ethyl acetate (2 mL) was slowly added m-chloroperbenzoic acid (70%, 82.0 mg, 0.332 mmol) at 0° C., and the mixture was stirred at the same temperature for 1.5 hr. The reaction mixture was poured into aqueous sodium bicarbonate solution, and the mixture was extracted with ethyl acetate. The extract was dried over sodium sulfate, and concentrated under reduced pressure. The residue was purified by silica gel column chromatography (hexane:ethyl acetate=90:10-35:65) to give methyl {(3S)-6-[(4-{2,6-dimethyl-4-[3-(methylsulfonyl)propoxy]phenyl}-2,3-dihydro-1H-inden-1-yl)(trifluoroacetyl)amino]-2,3-dihydro-1-benzofuran-3-yl}acetate (67.5 mg, yield 65%) as a colorless oil. To a mixed solution of methyl {(3S)-6-[(4-{2,6-dimethyl-4-[3-(methylsulfonyl)propoxy]phenyl}-2,3-dihydro-1H-inden-1-yl)(trifluoroacetyl)amino]-2,3-dihydro-1-benzofuran-3-yl}acetate (67.5 mg, 0.102 mmol) obtained above in tetrahydrofuran (1 mL) and methanol (0.5 mL) was added 1 M aqueous sodium hydroxide solution (0.614 mL), and the mixture was stirred at 50° C. for 2 hr. The reaction mixture was neutralized with 1 M hydrochloric acid (0.512 mL) and diluted with brine, and the mixture was extracted with ethyl acetate. The extract was washed with saturated brine, dried over anhydrous sodium sulfate, and concentrated under reduced pressure. The residue was dissolved in a mixed solution of tetrahydrofuran (1 mL) and methanol (0.5 mL), and 1 M aqueous sodium hydroxide solution (0.204 mL) was added. The reaction mixture was concentrated under reduced pressure, the residue was dissolved in water, and 1 M aqueous hydrochloric acid solution (0.204 mL) was slowly added dropwise. The obtained precipitate was collected by filtration, washed with water, and dried to give the title compound (48.6 mg, yield 86%) as a white solid.

¹H NMR (300 MHz, CDCl₃) δ 1.71-2.00 (7H, m), 2.25-2.69 (6H, m), 2.82 (1H, dd, J=16.7, 5.3 Hz), 2.97 (3H, s), 3.20-3.34 (2H, m), 3.71-3.87 (1H, m), 4.13 (2H, t, J=5.7 Hz), 4.27 (1H, dd, J=9.1, 6.1 Hz), 4.75 (1H, t, J=8.9 Hz), 5.02 (1H, t, J=6.4 Hz), 6.17-6.30 (2H, m), 6.65 (2H, s), 6.99 (2H, t, J=7.0 Hz), 7.18-7.40 (2H, m).

MS m/z 550 (M+H)⁺.

### Example 244

### methyl [(3S)-6-{[3-(2-ethoxy-6-fluoro-1H-benzimidazol-1-yl)-2-methylbenzyl]amino}-2,3-dihydro-1-benzofuran-3-yl]acetate

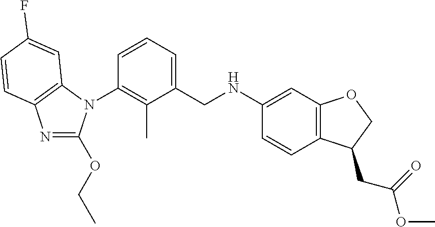

To a solution of [3-(2-ethoxy-6-fluoro-1H-benzimidazol-1-yl)-2-methylphenyl]methanol (1.8 g, 6.0 mmol) in acetonitrile (100 mL) was added Dess-Martin periodinane (2.8 g, 6.6 mmol) at 0° C., and the mixture was stirred at 0° C. for 1 hr. To the reaction mixture was added aqueous sodium hydrogen carbonate solution, and the mixture was stirred at room temperature for min. Then, to the reaction mixture was added aqueous sodium thiosulfate solution, and the mixture was stirred at room temperature for 30 min. The reaction mixture was extracted with ethyl acetate, and the extract was washed with saturated brine, dried over anhydrous magnesium sulfate, and concentrated under reduced pressure. The residue was purified by silica gel column chromatography (hexane:ethyl acetate=90:10-75:25) to give a solid (1.45 g, yield 81%). A solution of the obtained solid (0.388 g, 1.3 mmol), methyl [(3S)-6-amino-2,3-dihydro-1-benzofuran-3-yl]acetate (0.2 g, 0.97 mmol) and acetic acid (0.5 mL) in acetonitrile (6 mL) was stirred at room temperature for 1 hr, sodium triacetoxyborohydride (0.636 g, 3.0 mmol) was added, and the mixture was stirred at room temperature for 3 hr. A 1 M aqueous sodium hydroxide solution was poured into the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=90:10-80:20) to give the title compound (477 mg, yield quant.) as an oil.

¹H NMR (300 MHz, CDCl₃) δ1.36-1.43 (3H, m), 2.03 (3H, s), 2.50-2.58 (1H, m), 2.70-2.78 (1H, m), 3.70 (3H, s), 3.77-3.79 (1H, m), 4.08 (1H, br s), 4.22-4.25 (1H, m), 4.33 (2H, s), 4.53-4.65 (2H, m), 4.68-4.74 (1H, m), 6.13-6.19 (2H, m), 6.54-6.58 (1H, m), 6.87-6.96 (2H, m), 7.17-7.20 (1H, m), 7.28-7.33 (1H, m), 7.46-7.50 (2H, m).

### Example 245

### [(3S)-6-{[3-(2-ethoxy-6-fluoro-1H-benzimidazol-1-yl)-2-methylbenzyl]amino}-2,3-dihydro-1-benzofuran-3-yl]acetic acid trifluoroacetic acid salt

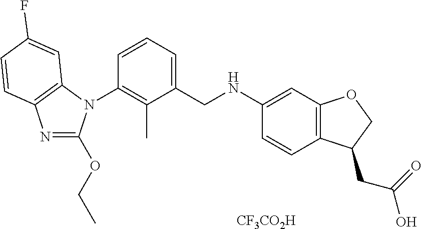

A mixed solution of methyl [(3S)-6-{[3-(2-ethoxy-6-fluoro-1H-benzimidazol-1-yl)-2-methylbenzyl]amino}-2,3-dihydro-1-benzofuran-3-yl]acetate (0.472 g, 0.96 mmol) and lithium hydroxide monohydrate (136 mg, 3.0 mmol) in tetrahydrofuran (20 mL) and water (20 mL) was stirred at room temperature for 1 hr. The reaction mixture was neutralized with 1 M hydrochloric acid, and the mixture was extracted with ethyl acetate. The extract was washed with saturated brine, dried over anhydrous magnesium sulfate, and concentrated under reduced pressure. The residue was purified by preparative HPLC using “Instrument: Gilson Inc. high throughput purification system

column: YMC Combiprep ODS-A, S-5 μm, 50×20 mm solvent: SOLUTION A; 0.1% trifluoroacetic acid-containing water, SOLUTION B; 0.1% trifluoroacetic acid-containing acetonitrile, gradient cycle: 0.00 min. (SOLUTION A/SOLUTION B=90/10), 1.00 min. (SOLUTION A/SOLUTION B=90/10), 4.20 min. (SOLUTION A/SOLUTION B=10/90), 5.40 min. (SOLUTION A/SOLUTION B=10/90), 5.50 min. (SOLUTION A/SOLUTION B=90/10), 5.60 min. (SOLUTION A/SOLUTION B=90/10), flow rate: mL/min, detection method: UV 220 nm”, and concentrated to give the title compound (0.0322 g, yield 6%) as a solid.

¹H NMR (300 MHz, DMSO-d₆) δ1.03-1.14 (3H, m), 1.86-1.94 (1H, m), 2.02 (3H, s), 2.39-2.45 (1H, m), 2.58-2.69 (1H, m), 3.58-3.62 (3H, m), 4.08-4.13 (1H, m), 4.26 (2H, s), 4.50-4.62 (2H, m), 6.07 (1H, s), 6.15 (1H, d, J=8.4 Hz), 6.42 (1H, d, J=6.6 Hz), 6.84-6.94 (2H, m), 7.03-7.07 (1H, m), 7.22-7.24 (1H, m), 7.32 (1H, t, J=7.2 Hz), 7.42 (1H, d, J=6.0 Hz), 11.15 (1H, s).

MS m/z 476 (M+H)⁺ (as free form).

### Example 246

### methyl {(3S)-6-[(2-methyl-3-{2-[(2R)-tetrahydrofuran-2-yl]-5-(trifluoromethyl)-1H-benzimidazol-1-yl}benzyl)amino]-2,3-dihydro-1-benzofuran-3-yl}acetate

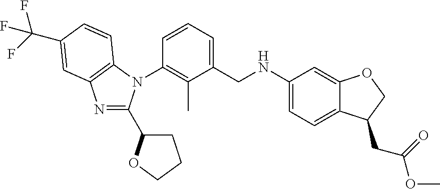

A solution of 2-methyl-3-{2-[(2R)-tetrahydrofuran-2-yl]-5-(trifluoromethyl)-1H-benzimidazol-1-yl}benzaldehyde (0.866 g, 2.31 mmol), methyl [(3S)-6-amino-2,3-dihydro-1-benzofuran-3-yl]acetate (0.414 g, 2.0 mmol) and acetic acid (1 mL) in acetonitrile (10 mL) was stirred at room temperature for 1 hr, sodium triacetoxyborohydride (1.27 g, 6.0 mmol) was added, and the mixture was stirred at room temperature for 1 hr. A 1 M aqueous sodium hydroxide solution was poured into the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=85:15-50:50) to give the title compound (930 mg, yield 82%) as a solid.

¹H NMR (300 MHz, CDCl₃) δ 1.89-1.99 (4H, m), 2.15-2.24 (2H, m), 2.51-2.59 (2H, m), 2.71-2.78 (1H, m), 3.71 (3H, s), 3.75-4.08 (3H, m), 4.11 (1H, br s), 4.21-4.26 (1H, m), 4.35 (2H, s), 4.73 (1H, t, J=9.0 Hz), 4.84-4.89 (1H, m), 6.13 (1H, s), 6.16-6.20 (1H, m), 6.95-7.02 (2H, m), 7.14-7.40 (2H, m), 7.47 (1H, d, J=8.7 Hz), 7.56 (1H, d, J=7.5 Hz), 8.13 (1H, s).

### Example 247

{(3S)-6-[(2-methyl-3-{2-[(2R)-tetrahydrofuran-2-yl]-5-(trifluoromethyl)-1H-benzimidazol-1-yl}benzyl)amino]-2,3-dihydro-1-benzofuran-3-yl}acetic acid

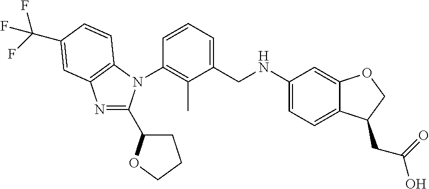

A mixed solution of methyl {(3S)-6-[(2-methyl-3-{2-[(2R)-tetrahydrofuran-2-yl]-5-(trifluoromethyl)-1H-benzimidazol-1-yl}benzyl)amino]-2,3-dihydro-1-benzofuran-3-yl}acetate (0.930 g, 1.64 mmol) and lithium hydroxide monohydrate (168 mg, 4.0 mmol) in tetrahydrofuran (30 mL) and water (25 mL) was stirred at room temperature for 1 hr. The reaction mixture was neutralized with 1 M hydrochloric acid, and the mixture was extracted with ethyl acetate. The extract was washed with saturated brine, dried over anhydrous magnesium sulfate, and concentrated under reduced pressure to give the title compound (0.820 g, yield 91%) as a solid.

¹H NMR (300 MHz, DMSO-d₆) δ 1.81-1.91 (3H, m), 1.99-2.05 (1H, m), 2.13-2.21 (1H, m), 2.39-2.50 (2H, m), 2.61-2.68 (1H, m), 3.56-3.62 (2H, m), 3.64-3.75 (2H, m), 4.07-4.12 (1H, m), 4.29 (2H, s), 4.60 (1H, t, J=9.0 Hz), 4.80-4.91 (1H, m), 6.05 (1H, s), 6.13-6.15 (2H, m), 6.92 (1H, d, J=8.1 Hz), 7.10-7.14 (1H, m), 7.32-7.42 (2H, m), 7.50-7.57 (2H, m), 8.13 (1H, s), 12.28 (1H, s).

### Example 248

### methyl {(3S)-6-[(2-methyl-3-{2-[(2R)-tetrahydrofuran-2-yl]-5-(trifluoromethoxy)-1H-benzimidazol-1-yl}benzyl)amino]-2,3-dihydro-1-benzofuran-3-yl}acetate

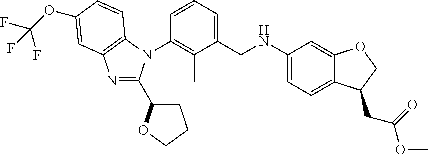

A solution of 2-methyl-3-{2-[(2R)-tetrahydrofuran-2-yl]-5-(trifluoromethoxy)-1H-benzimidazol-1-yl}benzaldehyde (0.530 g, 1.36 mmol), methyl [(3S)-6-amino-2,3-dihydro-1-benzofuran-3-yl]acetate (0.275 g, 1.33 mmol) and acetic acid (0.5 mL) in acetonitrile (6 mL) was stirred at room temperature for 1 hr, sodium triacetoxyborohydride (0.636 g, 3.0 mmol) was added, and the mixture was stirred at room temperature for 3 hr. A 1 M aqueous sodium hydroxide solution was poured into the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=80:20-50:50) to give the title compound (608 mg, yield 77%) as an oil.

¹H NMR (300 MHz, CDCl₃) δ 1.85-1.98 (5H, m), 2.12-2.25 (2H, m), 2.48-2.59 (2H, m), 2.71-2.78 (1H, m), 3.71 (3H, s), 3.74-4.00 (2H, m), 4.12 (1H, br s), 4.18-4.25 (1H, m), 4.34 (2H, s), 4.72 (1H, t, J=9.0 Hz), 4.84 (1H, t, J=6.6 Hz), 6.12 (1H, s), 6.16-6.19 (1H, m), 6.88-6.97 (2H, m), 7.08-7.38 (3H, m), 7.54-7.56 (1H, m), 7.71 (1H, s).

### Example 249

sodium {(3S)-6-[(2-methyl-3-{2-[(2R)-tetrahydrofuran-2-yl]-5-(trifluoromethoxy)-1H-benzimidazol-1-yl}benzyl)amino]-2,3-dihydro-1-benzofuran-3-yl}acetate

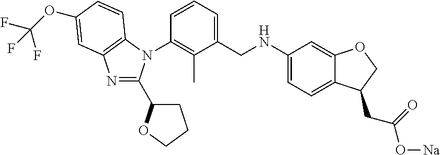

A mixed solution of methyl {(3S)-6-[(2-methyl-3-{2-[(2R)-tetrahydrofuran-2-yl]-5-(trifluoromethoxy)-1H-benzimidazol-1-yl}benzyl)amino]-2,3-dihydro-1-benzofuran-3-yl}acetate (0.600 g, 1.03 mmol) and lithium hydroxide monohydrate (84 mg, 2.0 mmol) in tetrahydrofuran (20 mL) and water (15 mL) was stirred at room temperature for 1 hr. The reaction mixture was neutralized with 1 M hydrochloric acid, and the mixture was extracted with ethyl acetate. The extract was washed with saturated brine, dried over anhydrous magnesium sulfate, and concentrated under reduced pressure to give a solid. The solid was dissolved in methanol (20 mL), sodium methoxide (50 mg, 0.93 mmol) was added, and the mixture was stirred at room temperature for 1 hr. The solvent was concentrated under reduced pressure to give the title compound (0.520 g, yield 86%) as a solid.

¹H NMR (300 MHz, DMSO-d₆) δ 1.81-1.90 (4H, m), 2.00-2.09 (1H, m), 2.11-2.18 (1H, m), 2.24-2.33 (1H, m), 2.43-2.58 (2H, m), 3.58-3.75 (3H, m), 4.07 (1H, t, J=8.1 Hz), 4.28 (2H, s), 4.59 (1H, t, J=9.0 Hz), 4.76-4.86 (1H, m), 6.03 (1H, s), 6.11-6.14 (2H, m), 6.87-6.92 (1H, m), 6.98-7.03 (1H, m), 7.21-7.24 (1H, m), 7.29-7.40 (2H, m), 7.49-7.52 (1H, m), 7.76 (1H, s).

### Example 250

### methyl {(3S)-6-[(3-{5-fluoro-2-[(2R)-tetrahydrofuran-2-yl]-1H-benzimidazol-1-yl}-2-methylbenzyl)amino]-2,3-dihydro-1-benzofuran-3-yl}acetate

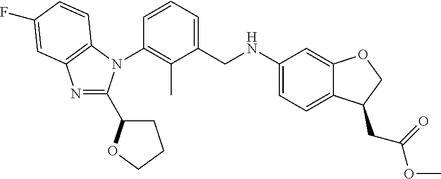

A solution of 3-{5-fluoro-2-[(2R)-tetrahydrofuran-2-yl]-1H-benzimidazol-1-yl}-2-methylbenzaldehyde (0.746 g, 2.3 mmol), methyl [(3S)-6-amino-2,3-dihydro-1-benzofuran-3-yl]acetate (0.414 g, 2.0 mmol) and acetic acid (1 mL) in acetonitrile (10 mL) was stirred at room temperature for 1 hr, sodium triacetoxyborohydride (1.27 g, 6.0 mmol) was added, and the mixture was stirred at room temperature for 1 hr. A 1 M aqueous sodium hydroxide solution was poured into the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=95:5-50:50) to give the title compound (1.0 g, yield 97%) as a solid.

¹H NMR (300 MHz, CDCl₃) δ 1.89-1.98 (4H, m), 2.16-2.24 (2H, m), 2.48-2.59 (2H, m), 2.71-2.78 (1H, m), 3.71 (3H, s), 3.75-3.87 (2H, m), 3.93-4.13 (2H, m), 4.21-4.26 (1H, m), 4.34 (2H, s), 4.69-4.75 (1H, m), 4.79-4.83 (1H, m), 6.13-6.19 (2H, m), 6.80-6.85 (1H, m), 6.93-7.16 (2H, m), 7.30-7.38 (2H, m), 7.49-7.55 (2H, m).

### Example 251

### {(3S)-6-[(3-{5-fluoro-2-[(2R)-tetrahydrofuran-2-yl]-1H-benzimidazol-1-yl}-2-methylbenzyl)amino]-2,3-dihydro-1-benzofuran-3-yl}acetic acid

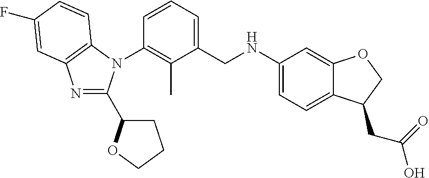

A mixed solution of methyl {(3S)-6-[(3-{5-fluoro-2-[(2R)-tetrahydrofuran-2-yl]-1H-benzimidazol-1-yl}-2-methylbenzyl)amino]-2,3-dihydro-1-benzofuran-3-yl}acetate (1.0 g, 1.94 mmol) and lithium hydroxide monohydrate (420 mg, 10.0 mmol) in tetrahydrofuran (100 mL) and water (60 mL) was stirred at room temperature for 1 hr. The reaction mixture was neutralized with 1 M hydrochloric acid, and the mixture was extracted with ethyl acetate. The extract was washed with saturated brine, dried over anhydrous magnesium sulfate, and concentrated under reduced pressure to give the title compound (0.280 g, yield 29%) as a solid.

¹H NMR (300 MHz, DMSO-d₆) δ 1.73-1.91 (4H, m), 1.99-2.16 (2H, m), 2.39-2.51 (2H, m), 2.60-2.68 (1H, m), 3.60-3.74 (3H, m), 4.01-4.12 (1H, m), 4.27 (2H, s), 4.60 (1H, t, J=9.0 Hz), 4.72-4.82 (1H, m), 6.04 (1H, s), 6.12-6.20 (2H, m), 6.87-6.93 (2H, m), 7.06-7.11 (1H, m), 7.28-7.40 (2H, m), 7.47-7.57 (2H, m), 12.27 (1H, br s).

### Example 252

### methyl {(3S)-6-[(3-{5-chloro-2-[(2R)-tetrahydrofuran-2-yl]-1H-benzimidazol-1-yl}-2-methylbenzyl)amino]-2,3-dihydro-1-benzofuran-3-yl}acetate

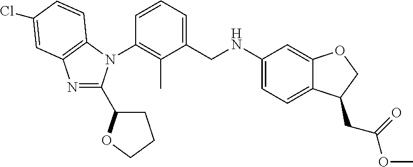

A solution of 3-{5-chloro-2-[(2R)-tetrahydrofuran-2-yl]-1H-benzimidazol-1-yl}-2-methylbenzaldehyde (0.784 g, 2.3 mmol), methyl [(3S)-6-amino-2,3-dihydro-1-benzofuran-3-yl]acetate (0.414 g, 2.0 mmol) and acetic acid (1 mL) in acetonitrile (10 mL) was stirred at room temperature for 0.5 hr. Sodium triacetoxyborohydride (1.27 g, 6.0 mmol) was added, and the mixture was stirred at room temperature for 1 hr. A 1 M aqueous sodium hydroxide solution was poured into the reaction mixture, and the mixture was extracted with ethyl acetate. The extract was washed with saturated brine, dried over anhydrous magnesium sulfate, and concentrated under reduced pressure. The residue was purified by silica gel column chromatography (hexane:ethyl acetate=85:15-50:50) to give the title compound (1.22 g, yield quant.) as a solid.

¹H NMR (300 MHz, CDCl₃) δ 1.88-1.95 (4H, m), 2.13-2.20 (2H, m), 2.49-2.59 (2H, m), 2.71-2.78 (1H, m), 3.71 (3H, s), 3.75-3.94 (2H, m), 4.08-4.15 (1H, m), 4.20-4.25 (1H, m), 4.34 (2H, s), 4.72 (1H, t, J=9.0 Hz), 4.80-4.85 (1H, m), 6.11-6.12 (1H, m), 6.16-6.19 (1H, m), 6.83 (1H, d, J=8.7 Hz), 6.94-6.97 (1H, m), 7.15-7.19 (2H, m), 7.26-7.34 (2H, m), 7.52-7.55 (1H, m), 7.80-7.815 (1H, m).

### Example 253

### {(3S)-6-[(3-{5-chloro-2-[(2R)-tetrahydrofuran-2-yl]-1H-benzimidazol-1-yl}-2-methylbenzyl)amino]-2,3-dihydro-1-benzofuran-3-yl}acetic acid

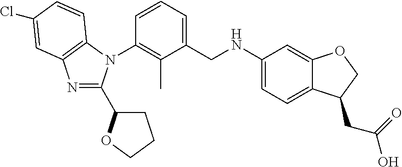

A mixed solution of methyl {(3S)-6-[(3-{5-chloro-2-[(2R)-tetrahydrofuran-2-yl]-1H-benzimidazol-1-yl}-2-methylbenzyl)amino]-2,3-dihydro-1-benzofuran-3-yl}acetate (1.22 g, 2.29 mmol) and 2 M aqueous sodium hydroxide solution (5 mL) in tetrahydrofuran (30 mL) and methanol (30 mL) was stirred at room temperature for 0.5 hr. The reaction mixture was concentrated, and neutralized with 1 M hydrochloric acid to allow precipitation of a solid. The obtained solid was dried under reduced pressure to give the title compound (0.880 g, yield 74%) as a white solid.

¹H NMR (300 MHz, DMSO-d₆) δ 1.80-1.91 (4H, m), 1.99-2.16 (2H, m), 2.39-2.50 (1H, m), 2.61-2.68 (1H, m), 3.60-3.70 (4H, m), 4.10 (1H, t, J=8.4 Hz), 4.27 (2H, s), 4.60 (1H, t, J=8.7 Hz), 4.74-4.85 (1H, m), 6.04 (1H, s), 6.12-6.15 (2H, m), 6.90-6.93 (2H, m), 7.23-7.50 (4H, m), 7.81 (1H, s), 12.25 (1H, br s).

### Formulation Example 1

### Production of Capsule

| | |
|---|---|
| 1) compound of Example 1 | 30 mg |
| 2) microcrystalline cellulose | 10 mg |
| 3) lactose | 19 mg |
| 4) magnesium stearate | 1 mg |
| total | 60 mg |

The above-mentioned 1), 2), 3) and 4) are mixed and filled in a gelatin capsule.

### Formulation Example 2

### Production of Tablet

| | |
|---|---|
| 1) compound of Example 1 | 30 g |
| 2) lactose | 50 g |
| 3) corn starch | 15 g |
| 4) carboxymethylcellulose calcium | 44 g |
| 5) magnesium stearate | 1 g |
| 1000 tablets total | 140 g |

The total amount of the above-mentioned 1), 2) and 3) and g of 4) are kneaded with water, vacuum dried and granulated. The granulated powder is mixed with 14 g of 4) and 1 g of 5) and tableted with a tableting machine. In this way, 1000 tablets containing 30 mg of the compound of Example 1 per tablet are obtained.

### Experimental Example 1

### Receptor Function Regulating Action (Agonist Action) on Human Derived GPR40

The CHO cell lines that stably expressed human derived GPR40 were used for the determination of the agonist activity. Unless particularly specified, the CHO cell lines were cultured in an α-MEM medium (Wako Pure Chemical Industries, Ltd.) containing 10% dialyzed fetal bovine serum (TRA Thermo Electron).

The cells cultured almost to confluent on the day before assay were rinsed with PBS (Invitrogen), detached with 0.5 mM EDTA (Wako Pure Chemical Industries, Ltd.), and recovered by centrifugation. The obtained cells were counted, diluted to 2×10⁵ cells per 1 mL medium, dispensed by 40 μL per well to a 384 well black clear bottomplate (PE Biosystems), and cultured overnight in a CO₂ incubator. Various test compounds were added to CHO cells prepared as mentioned above, and change of intracellular calcium concentration was measured using FLIPRtetra (Molecular Device). For the measurement by this device, a Calcium 4 assay kit (Molecular Devices: R8142) was used.

First, fatty acid free BSA at the final concentration of 0.1% was added to the buffer attached to the kit to prepare an assay buffer. Using the assay buffer, a loading solution with Fluo4 was prepared such that the final concentration of fatty acid free BSA was 0.1% and the final concentration of probenecid was 625 μM. On the other hand, a test compound was diluted with the assay buffer to a given concentration, and dispensed to the polypropylene 384-well plate (sample plate). The medium was removed from the cell culture plate, and the loading solution with Fluo4 was dispensed by 25 μL. After culture in a CO₂ incubator at 37° C. for 40 min, a sample plate and a cell plate were simultaneously set on the FLIPRtetra, various test compounds were added by 25 μL according to the program of the device, and change of intracellular calcium concentration was measured. The agonist activity of each compound at 1 μM was calculated as a relative activity value to the activity of 10 μM γ linolenic acid as 100% from the wave peak height. The results are shown in Table 1.

TABLE 1

| Example No. | activity (%) |
|---|---|
| 3 | 128 |
| 7 | 124 |
| 8 | 131 |
| 9 | 128 |
| 19 | 129 |
| 25 | 129 |
| 31 | 127 |
| 33 | 109 |
| 37 | 117 |
| 67 | 111 |
| 77-2 | 132 |
| 79 | 126 |
| 82 | 129 |
| 86 | 128 |
| 89 | 129 |
| 99 | 123 |
| 109 | 122 |
| 114 | 132 |
| 121 | 129 |
| 131 | 129 |
| 149 | 90 |
| 153 | 111 |
| 173 | 121 |
| 180 | 125 |
| 201 | 124 |
| 207 | 121 |
| 219 | 125 |
| 221 | 126 |
| 223 | 111 |
| 227 | 105 |
| 232 | 121 |
| 242 | 121 |

### Experimental Example 2

The blood glucose increase suppressive action and insulin secretagogue action of the compound of the present invention were evaluated by an oral glucose tolerance test using N-STZ-1.5 rat (TAKEDA RABICS).

### (1) Animal Used

Streptozotocin (120 mg/kg) was subcutaneously administered to male Wistar Kyoto rats (1-2 days old) to prepare N-STZ-1.5 rat which is a type 2 diabetes model. The rats were bred under free access to a feed (CE-2, sold by CLEA Japan, Inc.).

### (2) Experiment Method and Results

Male N-STZ-1.5 rats (21-week-old) were fasted for 19-21 hr, and the body weight was measured. Heparin (Ajinomoto Co., Inc.) was added as an anticoagulant, and aprotinin (SIGMA) was added as a protease inhibitor, and the blood was collected from the tail vein. 0.5% Methylcellulose (control group) or a 0.5% methylcellulose suspension of a test compound (1 mg/kg body weight) was orally administered to the rats of each group (6 per group) at 5 ml/kg and, 1 hr after the administration, a glucose solution (Otsuka Pharmaceutical Factory, Inc.) was orally administered at 1.5 g/5 ml/kg. The blood was collected from the tail vein immediately before glucose loading (0 min value) and 10, 30, 60, 120 min thereafter. The blood was centrifuged and the plasma was separated. The plasma glucose and insulin concentration of the obtained plasma were measured. The plasma glucose was measured by automatic analyzer 7080 (HITACHI), and the insulin concentration was measured by RAT INSULIN RIA KIT (LINCO Research). The glucose AUC and insulin AUC were calculated from the obtained values by the following formulas. Moreover, blood glucose lowering rate and insulin increasing rate were calculated from the obtained AUC by the following formulas. Statistical significance versus control was analyzed by Student's t-test or Aspin-Welch test. The results are shown in Table 2.

### Glucose AUC:

{(0 min plasma glucose)+(10 min plasma glucose)}×10/2+{(10 min plasma glucose)+(30 min plasma glucose)}×20/2+{(30 min plasma glucose)+(60 min plasma glucose)}×30/2+{(60 min plasma glucose)+(120 min plasma glucose)}×60/2

insulin AUC:

{(0 min plasma insulin)+(10 min plasma insulin)}×10/2+{(10 min plasma insulin)+(30 min plasma insulin)}×20/2+{(30 min plasma insulin)+(60 min plasma insulin)}×30/2+{(60 min plasma insulin)+(120 min plasma insulin)}×60/2

blood glucose lowering rate: [(test compound administration group glucose AUC/control group glucose AUC)−1]×100

insulin increasing rate: [(test compound administration group insulin AUC/control group glucose AUC)-1]×100

TABLE 2

| Compound | Blood glucose lowering rate | Insulin increasing rate |
|---|---|---|
| Ex. 7 | −20.5%** | +39.5%^{$} |
| Ex. 77-1 | −13.6%^{$} | +40.2%^{$} |

**p ≦ 0.01 (Student's t-test)
^{$}p ≦ 0.05 ( Aspin-Welch test)

### Experimental Example 3

The blood glucose increase suppressive action of the compound of the present invention was evaluated by an oral glucose tolerance test using Wistar fatty rat (TAKEDA RABICS).

### (1) Animal Used

The animal used was female Wistar fatty rat (prepared by TAKEDA RABICS, 18- to 22-week-old). The rats were bred under free access to a feed (CE-2, sold by CLEA Japan, Inc.).

### (2) Experiment Method and Results

Female Wistar fatty rats (18- to 22-week-old) were fasted for 19-21 hr, and the body weight was measured. Heparin (Ajinomoto Co., Inc.) was added as an anticoagulant, and aprotinin (SIGMA) was added as a protease inhibitor, and the blood was collected from the tail vein. 0.5% Methylcellulose (control group) or a 0.5% methylcellulose suspension of a test compound (1 mg/kg body weight) was orally administered to the rats of each group (6 per group) at 5 ml/kg and, 1 hr after the administration, a glucose solution (Otsuka Pharmaceutical Factory, Inc.) was orally administered at 1 g/5 ml/kg. The blood was collected from the tail vein immediately before glucose loading (0 min value) and 10, 30, 60, 120 min thereafter. The blood was centrifuged and the plasma was separated. The plasma glucose of the obtained plasma were measured by automatic analyzer 7080 (HITACHI). The glucose AUC was calculated from the obtained values by the formulas described in Experimental Example 2. Moreover, blood glucose lowering rate was calculated from the obtained AUC by the formulas described in Experimental Example 2. Statistical significance versus control was analyzed by Student's t-test. The results are shown in Table 3.

TABLE 3

| Compound | Blood glucose lowering rate |
|---|---|
| Ex. 9 | −15.8%** |
| Ex. 33 | −19.5%** |

**p ≦ 0.01 (Student's t-test)

### INDUSTRIAL APPLICABILITY

The compound of the present invention has a superior GPR40 receptor function regulating action and is useful as an insulin secretagogue or a prophylactic or therapeutic drug for diabetes and the like.

This application is based on U.S. provisional application Nos. 61/213,448 and 61/272,980, the contents of which are incorporated in full herein.

1. A compound represented by the formula (I): 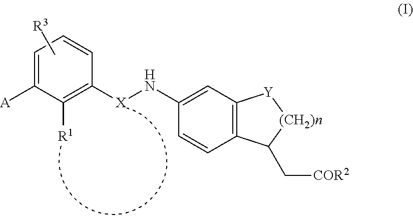
wherein R¹ is a halogen atom, hydroxy, optionally substituted C₁₋₆ alkyl or optionally substituted C₁₋₆ alkoxy,
R² is optionally substituted hydroxy,
R³ is a hydrogen atom, a halogen atom or optionally substituted C₁₋₆ alkyl,
X is CH₂ (wherein R¹ and X optionally form an optionally substituted ring),
Y is CH₂, NH or O,
Z is CH or N,
n is an integer selected from 1 to 3,
A is a halogen atom, optionally substituted amino, or a 4- to 13-membered cyclic group optionally substituted by 1 to 5 substituents selected from
(1) a halogen atom,
(2) optionally substituted amino,
(3) optionally substituted C₁₋₆ alkylthio,
(4) optionally substituted C₁₋₆ alkyl,
(5) optionally substituted C₃₋₁₀ cycloalkyl,
(6) optionally substituted C₁₋₆ alkoxy,
(7) optionally substituted C₆₋₁₄ aryl,
(8) an optionally substituted 4- to 7-membered heterocyclic group, and
(9) optionally substituted 4- to 7-membered heterocyclyl-oxy or a salt thereof.

2. The compound or salt according to claim 1, wherein R¹ is C₁₋₆ alkyl (wherein R¹ and X optionally form an optionally substituted ring).

3. The compound or salt according to claim 1, which is represented by the formula (II): 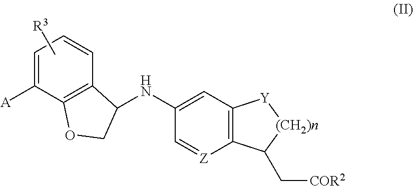
wherein R² is optionally substituted hydroxy,
R³ is a hydrogen atom, a halogen atom or optionally substituted C₁₋₆ alkyl,
Y is CH₂, NH or O,
Z is CH or N,
n is an integer selected from 1 to 3,
A is a halogen atom, optionally substituted amino, or a 4- to 13-membered cyclic group optionally substituted by 1 to 5 substituents selected from
(1) a halogen atom,
(2) optionally substituted amino,
(3) optionally substituted C₁₋₆ alkylthio,
(4) optionally substituted C₁₋₆ alkyl,
(5) optionally substituted C₃₋₁₀ cycloalkyl,
(6) optionally substituted C₁₋₆ alkoxy,
(7) optionally substituted C₆₋₁₄ aryl,
(8) an optionally substituted 4- to 7-membered heterocyclic group, and
(9) optionally substituted 4- to 7-membered heterocyclyl-oxy.

4. The compound or salt according to claim 1, wherein R² is hydroxy.

5. The compound or salt according to claim 1, wherein R³ is a hydrogen atom or C₁₋₆ alkyl.

6. The compound or salt according to claim 1, wherein Y is O.

7. The compound or salt according to claim 1, wherein Z is CH.

8. The compound or salt according to claim 1, wherein n is 1.

9. The compound or salt according to claim 1, wherein A is phenyl, benzimidazolyl or a 5- or 6-membered aromatic heterocyclic group, each of which is optionally substituted by 1 to 5 substituents selected from (a) a halogen atom,
(b) C₁₋₆ alkyl optionally substituted by 1 to 3 halogen atoms,
(c) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio,
(d) a 4- to 7-membered heterocyclic group, and
(e) 4- to 7-membered heterocyclyl-oxy optionally substituted by 1 to 3 substituents selected from C₁₋₆ alkyl and oxo.

10. The compound or salt according to claim 3, wherein R² is hydroxy,
R³ is a hydrogen atom or C₁₋₆ alkyl,
Y is O,
Z is CH,
n is 1,
A is phenyl, benzimidazolyl or a 5- or 6-membered aromatic heterocyclic group, each of which is optionally substituted by 1 to 5 substituents selected from
(a) a halogen atom,
(b) C₁₋₆ alkyl optionally substituted by 1 to 3 halogen atoms,
(c) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio,
(d) a 4- to 7-membered heterocyclic group, and
(e) 4- to 7-membered heterocyclyl-oxy optionally substituted by 1 to 3 substituents selected from C₁₋₆ alkyl and oxo.

11. The compound or salt according to claim 10, wherein A is phenyl optionally substituted by 1 to 3 substituents selected from
(a) C₁₋₆ alkyl optionally substituted by 1 to 3 halogen atoms,
(b) C₁₋₆ alkoxy optionally substituted by 1 to 3 substituents selected from
(1) a halogen atom,
(2) C₁₋₆ alkylsulfonyl,
(3) C₃₋₈cycloalkyl,
(4) mono- or di-C₁₋₆ alkyl-amino,
(5) C₁₋₆ alkoxy,
(6) C₆₋₁₄ aryl optionally substituted by a halogen atom,
(7) a 4- to 7-membered heterocyclic group optionally substituted by 1 to 3 substituents selected from C₁₋₆ alkyl and oxo, and
(8) C₁₋₆ alkylthio, and
(c) 4- to 7-membered heterocyclyl-oxy optionally substituted by 1 to 3 substituents selected from C₁₋₆ alkyl and oxo.

12. The compound or salt according to claim 10, wherein A is benzimidazolyl or a 5- or 6-membered aromatic heterocyclic group, each of which is optionally substituted by 1 to 3 substituents selected from
(a) a halogen atom,
(b) C₁₋₆ alkyl optionally substituted by 1 to 3 halogen atoms, and
(c) a 5- or 6-membered heterocyclic group.

13. [(3S)-6-{[(3S)-7-{2,6-Dimethyl-4-[3-(methylsulfonyl)propoxy]phenyl}-2,3-dihydro-1-benzofuran-3-yl]amino}-2,3-dihydro-1-benzofuran-3-yl]acetic acid or a salt thereof.

14. [(3S)-6-{[(3S)-7-{4-[(1,1-Dioxidotetrahydro-2H-thiopyran-4-yl)oxy]-2,6-dimethylphenyl}-2,3-dihydro-1-benzofuran-3-yl]amino}-2,3-dihydro-1-benzofuran-3-yl]acetic acid or a salt thereof.

15. [(3S)-6-{[(3S)-7-(2-Ethyl-6,7-difluoro-1H-benzimidazol-1-yl)-2,3-dihydro-1-benzofuran-3-yl]amino}-2,3-dihydro-1-benzofuran-3-yl]acetic acid or a salt thereof.

16. [(3S)-6-{[(3S)-7-(2-Ethoxy-6,7-difluoro-1H-benzimidazol-1-yl)-2,3-dihydro-1-benzofuran-3-yl]amino}-2,3-dihydro-1-benzofuran-3-yl]acetic acid or a salt thereof.

17. [(3S)-6-({(3S)-7-[4,6-Dimethyl-2-(morpholin-4-yl)pyrimidin-5-yl]-2,3-dihydro-1-benzofuran-3-yl}amino)-2,3-dihydro-1-benzofuran-3-yl]acetic acid or a salt thereof.

18. (canceled)

19. A medicament comprising the compound or salt according to claim 1.

20. The medicament according to claim 19 for activating a GPR40-mediated signal.

21. The medicament according to claim 19, which is an agent for the prophylaxis or treatment of diabetes or obesity.

22. A method of activating a GPR40-mediated signal, comprising administering the compound or salt according to claim 1 to a mammal.

23. A method for the prophylaxis or treatment of diabetes or obesity, comprising administering the compound or salt according to claim 1 to a mammal.

24-25. (canceled)
